(19) United States
(12) Patent Application Publication
(10) Pub. No.: US 2023/0212131 A1
(43) Pub. Date: Jul. 6, 2023

(54) COLLAGEN 1 TRANSLATION INHIBITORS AND METHODS OF USE THEREOF

(71) Applicant: Anima Biotech Inc., Bernardsville, NJ (US)

(72) Inventors: David William SHEPPARD, Ramat Gan (IL); Jason Paul TIERNEY, Ramat Gan (IL); Aviad MANDABI, Ramat Gan (IL); Wolfgang SCHMIDT, Essex (GB); Stefano LEVANTO, Essex (GB); Julie Nicole HAMBLIN, Essex (GB); Richard James BULL, Essex (GB); Iris ALROY, Ramat Gan (IL); Wissam MANSOUR, Ramat Gan (IL); Moty KLEPFISH, Ramat Gan (IL); Yaode WANG, Beijing (CN); Haitang LI, Beijing (CN)

(73) Assignee: Anima Biotech Inc., Bernardsville, NJ (US)

(21) Appl. No.: 17/928,283

(22) PCT Filed: Jun. 9, 2021
(86) PCT No.: PCT/US2021/036505

§ 371 Date: Nov. 29, 2022

Related U.S. Application Data
(60) Provisional application No. 63/036,458, filed on Jun. 9, 2020.

(51) Int. Cl. C07D 277/38; C07D 417/12; C07D 471/10; C07D 417/14

(52) U.S. Cl. CPC C07D 277/38; C07D 417/12; C07D 471/10; C07D 417/14

(57) ABSTRACT

The present invention relates to novel Collagen 1 translation inhibitors, composition and methods of preparation thereof, and uses thereof for treating Fibrosis including lung, liver, kidney, cardiac and dermal fibrosis, IPF, wound healing, scarring and Gingival fibromatosis, Systemic Sclerosis, and alcoholic and non-alcoholic steatohepatitis (NASH).

### FIELD OF THE INVENTION

The present invention relates to novel Collagen 1 translation inhibitors, composition and methods of preparation thereof, and uses thereof for treating Fibrosis including lung, liver, kidney, cardiac and dermal fibrosis, IPF, wound healing, scarring and gingival fibromatosis, Systemic Sclerosis, and alcoholic and non-alcoholic steatohepatitis (NASH).

### BACKGROUND OF THE INVENTION

The formation of fibrous connective tissue is part of the normal healing process following tissue damage due to injury or inflammation. During this process, activated immune cells including macrophages stimulate the proliferation and activation of fibroblasts, which in turn deposit connective tissue. However, abnormal or excessive production of connective tissue may lead to accumulation of fibrous material such that it interferes with the normal function of the tissue. Fibrotic growth can proliferate and invade healthy surrounding tissue, even after the original injury heals. Such abnormal formation of excessive connective tissue, occurring in a reparative or reactive process, is referred to as fibrosis.

Many agents cause activation of the fibrotic process and are released in response to tissue injury, inflammation and oxidative stress. Regardless of the initiating events, a feature common to all fibrotic diseases is the conversion of tissue resident fibroblast into ECM-producing myofibroblasts that secret collagen type I. Current programs indirectly target myofibroblast activation and collagen secretion by inhibiting a single fibrosis inducing signal.

Physiologically, fibrosis acts to deposit connective tissue, which can obliterate the architecture and function of the underlying organ or tissue. Defined by the pathological accumulation of extracellular matrix (ECM) proteins, fibrosis results in scarring and thickening of the affected tissue, which interferes with normal organ function. In various conditions, the formation of fibrotic tissue is characterized by the deposition of abnormally large amounts of collagen. The synthesis of collagen is also involved in a number of other pathological conditions. For example, clinical conditions and disorders associated with primary or secondary fibrosis, such as systemic sclerosis, graft-versus host disease (GVHD), pulmonary fibrosis and autoimmune disorders, are distinguished by excessive production of connective tissue, which results in the destruction of normal tissue architecture and function. These diseases can best be interpreted in terms of perturbations in cellular functions, a major manifestation of which is excessive collagen synthesis and deposition. The role of collagen in fibrosis has prompted attempts to develop drugs that inhibit its accumulation.

Excessive accumulation of collagen is the major pathologic feature in a variety of clinical conditions characterized by tissue fibrosis. These conditions include localized processes, as for example, pulmonary fibrosis and liver cirrhosis, or more generalized processes, like progressive systemic sclerosis. Collagen deposition is a feature of different forms of dermal fibrosis, which in addition to scleroderma, include localized and generalized morphea, keloids, hypertrophic scars, familial cutaneous collagenoma and connective tissue nevi of the collagen type. Recent advances in the understanding of the normal biochemistry of collagen have allowed us to define specific levels of collagen biosynthesis and degradation at which a pharmacologic intervention could lead to reduced collagen deposition in the tissues. Such compounds could potentially provide us with novel means to reduce the excessive collagen accumulation in diseases.

Fibrosis of the liver, also referred to herein as hepatic fibrosis, may be caused by various types of chronic liver injury, especially if an inflammatory component is involved. Self-limited, acute liver injury (e.g., acute viral hepatitis A), even when fulminant, does not necessarily distort the scaffolding architecture and hence does not typically cause fibrosis, despite loss of hepatocytes. However, factors such as chronic alcoholism, malnutrition, hemochromatosis, and exposure to poisons, toxins or drugs, may lead to chronic liver injury and hepatic fibrosis due to exposure to hepatotoxic chemical substances. Hepatic scarring, caused by surgery or other forms of injury associated with mechanical biliary obstruction, may also result in liver fibrosis.

Fibrosis itself is not necessarily symptomatic, however it can lead to the development of portal hypertension, in which scarring distorts blood flow through the liver, or cirrhosis, in which scarring results in disruption of normal hepatic architecture and liver dysfunction. The extent of each of these pathologies determines the clinical manifestation of hepato-fibrotic disorders. For example, congenital hepatic fibrosis affects portal vein branches, largely sparing the parenchyma. The result is portal hypertension with sparing of hepatocellular function.

### Treatment

Attempts to develop anti-fibrotic agents for the treatment of various disorders have been reported. However, treatment of established fibrosis, formed after months or years of chronic or repeated injury, still remains a challenge.

Treatments aimed at reversing the fibrosis are usually too toxic for long-term use (e.g. corticosteroids, penicillamine) or have no proven efficacy (e.g. colchicine).

Many patients do not respond to available treatments for fibrotic disorders, and long-term treatment is limited by toxicity and side effects. Therefore, a need remains for developing therapeutic modalities aimed at reducing fibrosis. The development of safe and effective treatments for established cirrhosis and portal hypertension and for attenuating fibrosis would be highly beneficial.

Attempts to treat idiopathic pulmonary fibrosis (IPF) with a combination of anti-inflammatory drugs (prednisone, azathioprine and N-acetyl-1-cysteine (NAC)), failed to improve outcomes, and instead increased mortality. In 2014, two drugs, pirfenidone, a drug with poorly understood mechanisms, and nintedanib, a tyrosine kinase inhibitor, were approved for the treatment of IPF mainly on the basis of their ability to reduce the decrease in forced vital capacity (FVC) and to slow the pace of disease progression. To date, however, it is unclear whether these drugs improve symptoms such as dyspnoea and cough, or whether their beneficial effect on functional decline translates to increased survival.

The compounds of this invention target activated fibroblasts and collagen over production and can therefore be used for treating fibrosis, including primary or secondary fibrosis, such as systemic sclerosis, graft-versus host disease (GVHD), pulmonary fibrosis and autoimmune disorders, lung fibrosis and idiopathic pulmonary fibrosis (IPF), as well as localized processes, as for example, pulmonary fibrosis and liver cirrhosis, or more generalized processes, like progressive systemic sclerosis. The compounds can be further useful in the treatment of different forms of dermal fibrosis, which in addition to scleroderma, include localized and generalized morphea, keloids, hypertrophic scars, familial cutaneous collagenoma and connective tissue nevi of the collagen type. The compounds can be further useful in the treatment of lung fibrosis and idiopathic pulmonary fibrosis (IPF), as well as hepatic fibrosis, resulting from hepatic scarring, caused by surgery or other forms of injury associated with mechanical biliary obstruction. Such fibrosis can lead to portal hypertension, in which scarring distorts blood flow through the liver, or cirrhosis as well as other hepato-fibrotic disorders including Non-alcoholic steatohepatitis (NASH), and alcoholic steatohepatitis (ASH), non-alcoholic fatty liver disease (NAFLD) and alcoholic fatty liver disease (AFLD), which can be similarly be treated by compounds of the invention.

### SUMMARY OF THE INVENTION

This invention provides a compound or its pharmaceutically acceptable salt, stereoisomer, tautomer, hydrate, N-oxide, reverse amide analog, prodrug, isotopic variants (e.g., deuterated analog), PROTAC, pharmaceutical product or any combination thereof, represented by the structure of formula I-VIII, and by the structures listed in Table 1, as defined herein below. In various embodiments, the compound is a Collagen I translation inhibitor.

This invention further provides a pharmaceutical composition comprising a compound or its pharmaceutically acceptable salt, stereoisomer, tautomer, hydrate, N-oxide, prodrug, isotopic variants (e.g., deuterated analog), PROTAC, pharmaceutical product or any combination thereof, represented by the structure of formula I-VIII, and by the structures listed in Table 1, as defined herein below, and a pharmaceutically acceptable carrier.

This invention further provides a method of treating, suppressing, reducing the severity, reducing the risk of developing or inhibiting fibrosis in a subject, comprising administering a compound represented by the structure of formula I-VIII, and by the structures listed in Table 1, as defined herein below, to a subject suffering from fibrosis under conditions effective to treat, suppress, reduce the severity, reduce the risk of developing, or inhibit fibrosis in said subject. In some embodiments, the fibrosis is a systemic fibrotic disease. In some embodiments, the systemic fibrotic disease is systemic sclerosis, multifocal fibrosclerosis (IgG4-associated fibrosis), nephrogenic systemic fibrosis, sclerodermatous graft vs. host disease, or any combination thereof. In some embodiments, the fibrosis is an organ-specific fibrotic disease. In some embodiments, the organ-specific fibrotic disease is lung fibrosis, cardiac fibrosis, kidney fibrosis, pulmonary fibrosis, liver and portal vein fibrosis, radiation-induced fibrosis, bladder fibrosis, intestinal fibrosis, peritoneal sclerosis, diffuse fasciitis, wound healing, scaring, or any combination thereof. In some embodiments, the lung fibrosis is idiopathic pulmonary fibrosis (IPF). In some embodiments, the cardiac fibrosis is hypertension-associated cardiac fibrosis, Post-myocardial infarction, Chagas disease-induced myocardial fibrosis or any combination thereof. In some embodiments, the kidney fibrosis is diabetic and hypertensive nephropathy, urinary tract obstruction-induced kidney fibrosis, inflammatory/autoimmune-induced kidney fibrosis, aristolochic acid nephropathy, polycystic kidney disease, or any combination thereof. In some embodiments, the pulmonary fibrosis is idiopathic pulmonary fibrosis, silica-induced pneumoconiosis (silicosis), asbestos-induced pulmonary fibrosis (asbestosis), chemotherapeutic agent-induced pulmonary fibrosis, or any combination thereof. In some embodiments, the liver and portal vein fibrosis is alcoholic and nonalcoholic liver fibrosis, hepatitis C-induced liver fibrosis, primary biliary cirrhosis, parasite-induced liver fibrosis (schistosomiasis), or any combination thereof. In some embodiments, the diffuse fasciitis is localized scleroderma, keloids, dupuytren's disease, peyronie's disease, myelofibrosis, oral submucous fibrosis, or any combination thereof. In some embodiments, the fibrosis is primary or secondary fibrosis. In some embodiments, the fibrosis is a result of systemic sclerosis, graft-versus host disease (GVHD), pulmonary fibrosis, autoimmune disorder, tissue injury, inflammation, oxidative stress or any combination thereof. In some embodiments, the fibrosis is hepatic fibrosis, lung fibrosis or dermal fibrosis. In some embodiments, the subject has a liver cirrhosis. In some embodiments, the dermal fibrosis is scleroderma. In some embodiments, the dermal fibrosis is a result of a localized or generalized morphea, keloids, hypertrophic scars, familial cutaneous collagenoma, connective tissue nevi of the collagen type, or any combination thereof. In some embodiments, the hepatic fibrosis is a result of hepatic scarring or chronic liver injury. In some embodiments, the chronic liver injury results from alcoholism, malnutrition, hemochromatosis, exposure to poisons, toxins or drugs.

This invention further provides a method of treating, suppressing, reducing the severity, reducing the risk of developing or inhibiting lung fibrosis in a subject, comprising administering a compound represented by the structure of formula I-VIII, and by the structures listed in Table 1, as defined herein below, to a subject suffering from lung fibrosis under conditions effective to treat, suppress, reduce the severity, reduce the risk of developing, or inhibit lung fibrosis in said subject. In some embodiments, the lung fibrosis is idiopathic pulmonary fibrosis (IPF).

This invention further provides a method of treating, suppressing, reducing the severity, reducing the risk of developing or inhibiting idiopathic pulmonary fibrosis (IPF) in a subject, comprising administering a compound represented by the structure of formula I-VIII, and by the structures listed in Table 1, as defined herein below, to a subject suffering from idiopathic pulmonary fibrosis (IPF) under conditions effective to treat, suppress, reduce the severity, reduce the risk of developing, or inhibit idiopathic pulmonary fibrosis (IPF) in said subject.

This invention further provides a method of treating, suppressing, reducing the severity, reducing the risk of developing or inhibiting hepato-fibrotic disorder in a subject, comprising administering a compound represented by the structure of formula I-VIII, and by the structures listed in Table 1, as defined herein below, to a subject suffering from hepato-fibrotic disorder under conditions effective to treat, suppress, reduce the severity, reduce the risk of developing, or inhibit hepato-fibrotic disorder in said subject. In some embodiments, the hepato-fibrotic disorder is a portal hypertension, cirrhosis, congenital hepatic fibrosis or any combination thereof.

This invention further provides a method of treating, suppressing, reducing the severity, reducing the risk of developing or inhibiting cirrhosis in a subject, comprising administering a compound represented by the structure of formula I-VIII, and by the structures listed in Table 1, as defined herein below, to a subject suffering from cirrhosis under conditions effective to treat, suppress, reduce the severity, reduce the risk of developing, or inhibit cirrhosis in said subject. In some embodiments, the cirrhosis is a result of hepatitis or alcoholism.

This invention further provides a method of treating, suppressing, reducing the severity, reducing the risk of developing or inhibiting alcoholic steatohepatitis (ASH) in a subject, comprising administering a compound represented by the structure of formula I-VII, and by the structures listed in Table 1, as defined herein below, to a subject suffering from alcoholic steatohepatitis (ASH) under conditions effective to treat, suppress, reduce the severity, reduce the risk of developing, or inhibit the alcoholic steatohepatitis (ASH) in said subject.

This invention further provides a method of treating, suppressing, reducing the severity, reducing the risk of developing or inhibiting a non-alcoholic steatohepatitis (NASH) in a subject, comprising administering a compound represented by the structure of formula I-VIII, and by the structures listed in Table 1, as defined herein below, to a subject suffering from non-alcoholic steatohepatitis (NASH) under conditions effective to treat, suppress, reduce the severity, reduce the risk of developing, or inhibit the non-alcoholic steatohepatitis (NASH) in said subject.

This invention further provides a method of treating, suppressing, reducing the severity, reducing the risk of developing or inhibiting an alcoholic fatty liver disease (AFLD) in a subject, comprising administering a compound represented by the structure of formula I-VIII, and by the structures listed in Table 1, as defined herein below, to a subject suffering from alcoholic fatty liver disease (AFLD) under conditions effective to treat, suppress, reduce the severity, reduce the risk of developing, or inhibit the alcoholic fatty liver disease (AFLD) in said subject.

This invention further provides a method of treating, suppressing, reducing the severity, reducing the risk of developing or inhibiting a non alcoholic fatty liver disease (NAFLD) in a subject, comprising administering a compound represented by the structure of formula I-VIII, and by the structures listed in Table 1, as defined herein below, to a subject suffering from non alcoholic fatty liver disease (NAFLD) under conditions effective to treat, suppress, reduce the severity, reduce the risk of developing, or inhibit the non alcoholic fatty liver disease (NAFLD) in said subject.

This invention further provides a method of treating, suppressing, reducing the severity, reducing the risk of developing or inhibiting an autoimmune disease or disorder in a subject, comprising administering a compound represented by the structure of formula I-VII, and by the structures listed in Table 1, as defined herein below, to a subject suffering from an autoimmune disease or disorder under conditions effective to treat, suppress, reduce the severity, reduce the risk of developing, or inhibit the autoimmune disease or disorder in said subject.

This invention further provides a method of treating, suppressing, reducing the severity, reducing the risk of developing or inhibiting an autoimmune disease or disorder in a subject, comprising administering a compound represented by the structure of formula I-VIII, and by the structures listed in Table 1, as defined herein below, to a subject suffering from an autoimmune disease or disorder under conditions effective to treat, suppress, reduce the severity, reduce the risk of developing, or inhibit the autoimmune disease or disorder in said subject.

### BRIEF DESCRIPTION OF THE DRAWINGS

The patent of application file contains at least one drawing executed in color. Copies of this patent or patent application publication with color drawing(s) will be provided by the office upon request and payment of the necessary fee.

FIG. 1 demonstrates how Protein synthesis monitoring (PSM) specifically monitors collagen 1 synthesis. The assay system comprises human lung fibroblast cell line, WI-38 cells, which are activated to produce higher levels of collagen. Two tRNAs (di-tRNA) which decode one specific glycine codon and one specific proline codon were transfected with control RNAi or an RNAi directed to Collagen 1. The FRET signal specifically monitors collagen 1 translation, as the FRET signal in collagen 1-targeted siRNA treated cells is inhibited by 90%. In gray, cell nuclei stained with DAPI; In white, FRET signals from tRNA pair which decodes glycine-proline di-codons.

FIG. 2 depicts that hits selectively regulate collagen translation. In the upper panel, the Y-axis depicts normalized values of metabolic labeling in control cells. Only compounds which showed minimal effects on global protein synthesis (±20% of control) and minimal effects on collagen 1 protein accumulation in W138 cells by di-tRNA Collagen FRET and by Collagen 1 specific immunofluorescence were selected as compounds which selectively regulate collagen synthesis; In the lower panel, Y axis shows the FRET score for the collagen specific di-tRNA (PSM score) and the X-axis shows the normalized immunofluorescence values (relative to control). Compounds that show high PSM score are marked by dot size.

FIG. 3 demonstrate that Collagen translation modulator compound 327 is tissue selective

FIG. 4 demonstrate that compound 327 act at the level of translation. FIG. 4A: WI-38 Human Lung Fibroblasts, 96 hours incubation with compounds. White: Collagen type-I; Gray: DAPI. Immunofluorescence. FIG. 4B: WI-38 Human Lung Fibroblasts, 24 hours incubation with compounds. FISH analysis. White: Col-I mRNA; Gray: DAPI.

FIGS. 5A and 5B demonstrate the efficacy and toxicity of compounds 367, 365, 339 and 366. FIG. 5A depicts the pEC₅₀ of efficacy plotted against pEC₅₀ of toxicity. Dashed lines represent ×10 or ×100 window between efficacy and toxicity. FIG. 5B depicts representative images from compound 365. Images were taken with ×20 objective in Operetta machine (Perkin-Elmer). White: Collagen type-I; Grey: DAPI.

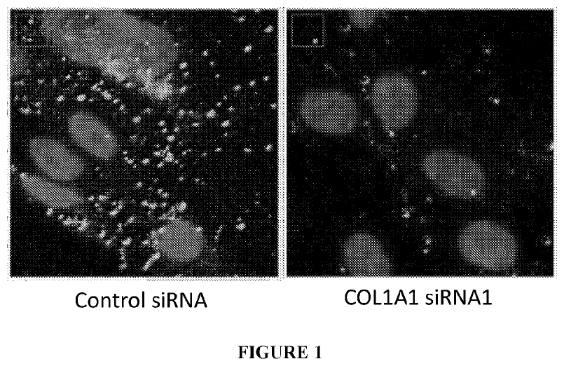

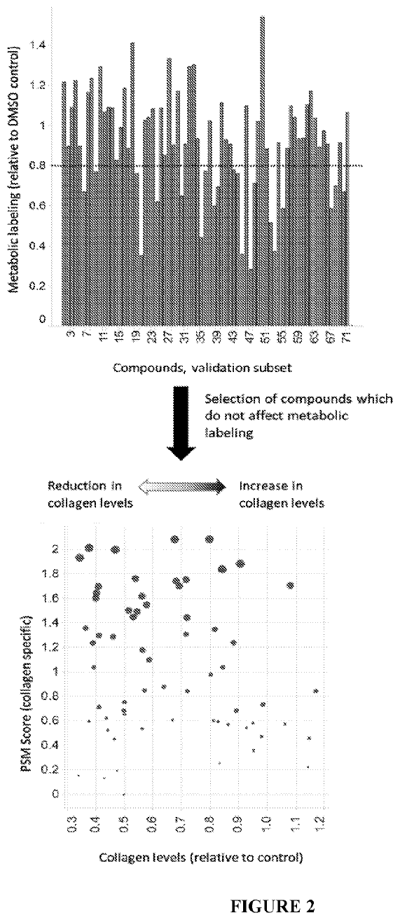

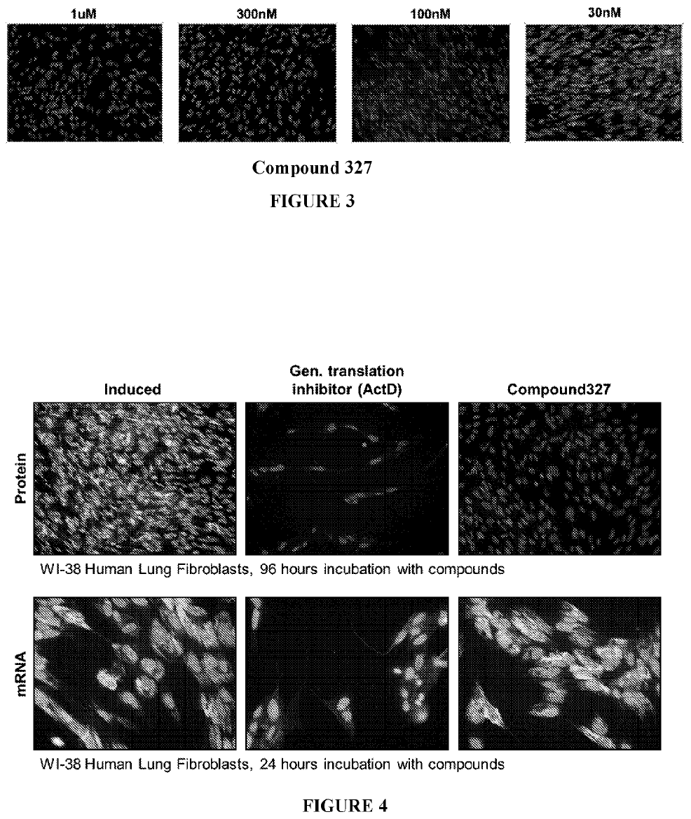

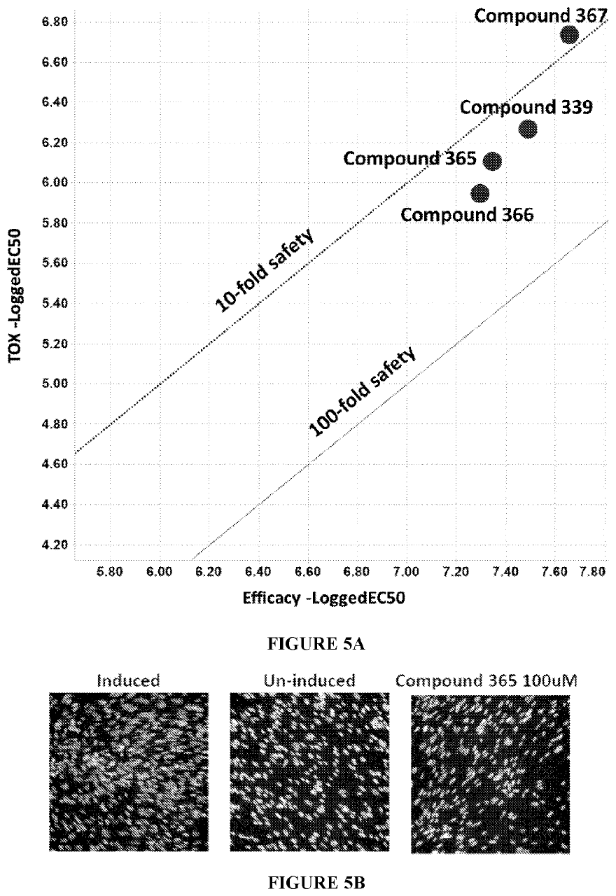

### DETAILED DESCRIPTION OF THE INVENTION

In various embodiments, this invention is directed to a compound represented by the structure of formula I:

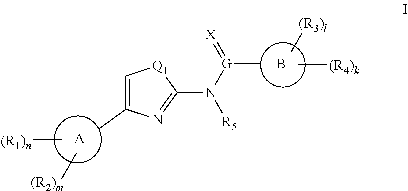

wherein

A and B rings are each independently a single or fused aromatic or heteroaromatic ring system (e.g., A: phenyl, thiophene, imidazole, pyrazole, pyrimidine, 2-, 3- or 4-pyridine, benzimidazole, indole, benzothiazole, benzooxazole, imidazopyridin, pyrazolopyridine, pyrrolopyridine, pyridazine, or pyrazine; B: phenyl, pyrimidine, 2-, 3- or 4-pyridine, pyridazine or pyrazine, thiophene, thiazole, pyrrole, imidazole, indazole), or a single or fused C₃-C₁₀ cycloalkyl (e.g. A: pyrrolidin-2-one; B: bicyclo[1.1.1]pentyl, cyclobutyl, cyclohexyl, cyclopentyl) or a single or fused C₃-C₁₀ heterocyclic ring (e.g., morpholine, piperidine, piperazine, tetrahydro-2H-pyran, azetidine, pyrrolidin-2-one);

R₁ and R₂ are each independently H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl) or substituted or unsubstituted benzyl; or R₂ and R₁ are joined together to form a 5 or 6 membered substituted or unsubstituted, aliphatic or aromatic, carbocyclic (e.g., benzene) or heterocyclic (e.g., 1,4-dioxane, 2,3-dihydro-1,4-dioxine, dioxol, dioxolpyridine) ring; R₃ and R₄ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)₂₀—CH₃), C(O)N(R₁₀)(R₁₁) (e.g., C(O)-piperidine, C(O)-pyrrolidine, C(O)N(CH₃)₂, C(O)-piperazine), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, N-methylpiperazine-1-carboxamide piperidin-4-ol, piperidin-3-ol, morpholine, 3-methylmorpholine, 3-hydroxypiperidine, tetrahydro-2H-pyrane, tetrahydro-2H-thiopyran 1,1-dioxide, pyrazole, thiazole, imidazole, pyrrolidine, pyrrolidinone, octahydropyrrolo[1,2-α]pyrazine, 6-methyl-2,6-diazaspiro[3.3]heptane, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl) or substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

R₅ is H, R₂₀, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, or substituted or unsubstituted benzyl;

Q₁ is NH, S, or O;

G=X is C═O, C═S, S═O or SO₂;

R is H, OH, F, Cl, Br, I, CN, CF₃, NO₂, NH₂, NH(R₁₀) (e.g., NH(CH₃)), N(R₁₀)(R₁₁), R₂₀, C₁-C₅ linear or branched, C₁-C₅ substituted or unsubstituted alkyl (e.g., methyl, ethyl, CH₂CH₂OH, CH₂CH₂OCH₃), R₈—R₁₀ (e.g., CH₂—OH, CH₂CH₂—OH), C(O)—R₁₀ (e.g., C(O)-methylpyrroldine, C(O)-methylpiperidine, C(O)—CH₃), C₁-C₅ substituted or unsubstituted C(O)-alkyl (e.g., C(O)—CH₂CH₂—OCH₃, C(O)—CH₃, C(O)—CH₂—N(CH₃)₂, C(O)—CH₂—CH₂—N(CH₃)₂, C(O)—CH₂—OH), C(O)—R₈—R₁₀ (e.g., C(O)—CH₂CH₂—OH), C(O)-substituted or unsubstituted C₃-C₈ heterocyclic ring (e.g., C(O)-methylpyrroldine, C(O)-methylpiperidine), C₁-C₅ substituted or unsubstituted SO₂-alkyl (e.g., SO₂—CH₃), C₁-C₅ substituted or unsubstituted C(O)—NH-alkyl (e.g., C(O)—NH—CH₃), C₁-C₅ linear or branched C(O)—O-alkyl (e.g., C(O)—O-tBu), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), or substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, OH, substituted or unsubstituted C₁-C₅ linear or branched alkyl (e.g., methyl, ethyl, CH₂—CH₂—O—CH₃), C₁-C₅ linear or branched alkoxy (e.g., 0-CH₃), substituted or unsubstituted C₃-C₈ heterocyclic ring (e.g., 1-(methylsulfonyl)piperidine, 1-(methylsulfonyl)piperazine, tetrahydro-2H-pyrane, morpholine, thiomorpholine 1,1-dioxide, methyl-pyrrolidine, methyl-piperidine), C(O)-alkyl, or S(O)₂-alkyl;

or R₁₀ and R₁₁ are joined to form a substituted or unsubstituted C₃-C₈ heterocyclic ring (e.g., morpholine, piperazine, piperidine, pyrrolidine, 1-methylpyrrolidin-2-one, oxetane, azetidine, 1-methylazetidine); R₂₀ is represented by the following structure:

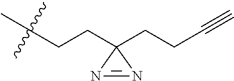 [SMILES: *CCC1(CCC#C)N=N1]

wherein substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof;

n and l are each independently an integer between 1 and 3 (e.g., 1 or 2);

m and k are each independently an integer between 0 and 3 (e.g., 0);

or its pharmaceutically acceptable salt, stereoisomer, tautomer, hydrate, N-oxide, prodrug, isotopic variant (e.g., deuterated analog), PROTAC, reverse amide, pharmaceutical product or any combination thereof.

In various embodiments, this invention is directed to a compound represented by the structure of formula II:

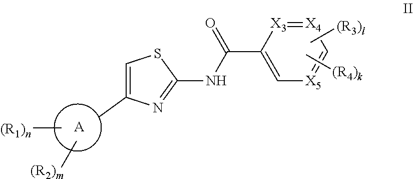

wherein

A ring is single or fused aromatic or heteroaromatic ring system (e.g., phenyl, thiophene, imidazole, pyrazole, pyrimidine, 2-, 3- or 4-pyridine, benzimidazole, indole, benzothiazole, benzooxazole, imidazopyridin, pyrazolopyridine, pyrrolopyridine, pyridazine, or pyrazine), or a single or fused C₃-C₁₀ cycloalkyl (e.g. pyrrolidin-2-one) or a single or fused C₃-C₁₀ heterocyclic ring (e.g., morpholine, piperidine, piperazine, tetrahydro-2H-pyran, azetidine, pyrrolidin-2-one);

R₁ and R₂ are each independently H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl), or substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., 1,4-dioxane, 2,3-dihydro-1,4-dioxine, dioxol, dioxolpyridine) ring;

R₃ and R₄ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)₂O—CH₃), C(O)N(R₁₀)(R₁₁) (e.g., C(O)-piperidine, C(O)-pyrrolidine, C(O)N(CH₃)₂, C(O)-piperazine), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, N-methylpiperazine-1-carboxamide piperidin-4-ol, piperidin-3-ol, morpholine, 3-methylmorpholine, 3-hydroxypiperidine, tetrahydro-2H-pyrane, tetrahydro-2H-thiopyran 1,1-dioxide, pyrazole, thiazole, imidazole, pyrrolidine, pyrrolidinone, octahydropyrrolo[1,2-a]pyrazine, 6-methyl-2,6-diazaspiro[3.3]heptane, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl), or substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

X₃, X₄ and X₅ are each independently C or N;

R is H, OH, F, Cl, Br, I, CN, CF₃, NO₂, NH₂, NH(R₁₀) (e.g., NH(CH₃)), N(R₁₀)(R₁₁), R₂₀, C₁-C₅ linear or branched, C₁-C₅ substituted or unsubstituted alkyl (e.g., methyl, ethyl, CH₂CH₂OH, CH₂CH₂OCH₃), R₈—R₁₀ (e.g., CH₂—OH, CH₂CH₂—OH), C(O)—R₁₀ (e.g., C(O)-methylpyrroldine, C(O)-methylpiperidine, C(O)—CH₃), C₁-C₅ substituted or unsubstituted C(O)-alkyl (e.g., C(O)—CH₂CH₂—OCH₃, C(O)—CH₃, C(O)—CH₂—N(CH₃)₂, C(O)—CH₂—CH₂—N(CH₃)₂, C(O)—CH₂—OH), C(O)—R₈—R₁₀ (e.g., C(O)—CH₂CH₂—OH), C(O)-substituted or unsubstituted C₃-C₈ heterocyclic ring (e.g., C(O)-methylpyrroldine, C(O)-methylpiperidine), C₁-C₅ substituted or unsubstituted SO₂-alkyl (e.g., SO₂—CH₃), C₁-C₅ substituted or unsubstituted C(O)—NH-alkyl (e.g., C(O)—NH—CH₃), C₁-C₅ linear or branched C(O)—O-alkyl (e.g., C(O)—O-tBu), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), or substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]q, [C]_{q}
wherein q is between 2 and 10;

R₁₀ and R₁₁ are each independently H, OH, substituted or unsubstituted C₁-C₅ linear or branched alkyl (e.g., methyl, ethyl, CH₂—CH₂—O—CH₃), C₁-C₅ linear or branched alkoxy (e.g., O—CH₃), substituted or unsubstituted C₃-C₈ heterocyclic ring (e.g., 1-(methylsulfonyl)piperidine, 1-(methylsulfonyl)piperazine, tetrahydro-2H-pyrane, morpholine, thiomorpholine 1,1-dioxide, methyl-pyrrolidine, methyl-piperidine), C(O)-alkyl, or S(O)₂-alkyl;

or R₁₀ and R₁₁ are joined to form a substituted or unsubstituted C₃-C₈ heterocyclic ring (e.g., morpholine, piperazine, piperidine, pyrrolidine, 1-methylpyrrolidin-2-one, oxetane, azetidine, 1-methylazetidine),

R₂₀ is represented by the following structure:

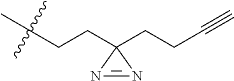 [SMILES: *CCC1(CCC#C)N=N1]

wherein substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof;

n and l are each independently an integer between 1 and 3 (e.g., 1 or 2);

m and k are each independently an integer between 0 and 3 (e.g., 0);

or its pharmaceutically acceptable salt, stereoisomer, tautomer, hydrate, N-oxide, prodrug, isotopic variant (e.g., deuterated analog), PROTAC, reverse amide, pharmaceutical product or any combination thereof.

In various embodiments, this invention is directed to a compound represented by the structure of formula III:

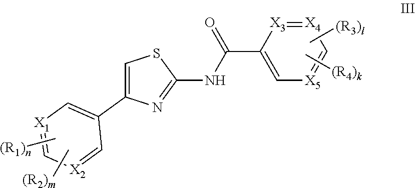

wherein

R₁ and R₂ are each independently H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl), or substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., 1,4-dioxane, 2,3-dihydro-1,4-dioxine, dioxol, dioxolpyridine) ring;

R₃ and R₄ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)₂₀—CH₃), C(O)N(R₁₀)(R₁₁) (e.g., C(O)-piperidine, C(O)-pyrrolidine, C(O)N(CH₃)₂, C(O)-piperazine), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, N-methylpiperazine-1-carboxamide piperidin-4-ol, piperidin-3-ol, morpholine, 3-methylmorpholine, 3-hydroxypiperidine, tetrahydro-2H-pyrane, tetrahydro-2H-thiopyran 1,1-dioxide, pyrazole, thiazole, imidazole, pyrrolidine, pyrrolidinone, octahydropyrrolo[1,2-a]pyrazine, 6-methyl-2,6-diazaspiro[3.3]heptane, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl), or substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

X₁, X₂ X₃, X₄ and X₅ are each independently C or N;

R is H, OH, F, Cl, Br, I, CN, CF₃, NO₂, NH₂, NH(R₁₀) (e.g., NH(CH₃)), N(R₁₀)(R₁₁), R₂₀, C₁-C₅ linear or branched, C₁-C₅ substituted or unsubstituted alkyl (e.g., methyl, ethyl, CH₂CH₂OH, CH₂CH₂OCH₃), R₈—R₁₀ (e.g., CH₂—OH, CH₂CH₂—OH), C(O)—R₁₀ (e.g., C(O)-methylpyrroldine, C(O)-methylpiperidine, C(O)—CH₃), C₁-C₅ substituted or unsubstituted C(O)-alkyl (e.g., C(O)—CH₂CH₂—OCH₃, C(O)—CH₃, C(O)—CH₂—N(CH₃)₂, C(O)—CH₂—CH₂—N(CH₃)₂, C(O)—CH₂—OH), C(O)—R₈—R₁₀ (e.g., C(O)—CH₂CH₂—OH), C(O)-substituted or unsubstituted C₃-C₈ heterocyclic ring (e.g., C(O)-methylpyrroldine, C(O)-methylpiperidine), C₁-C₅ substituted or unsubstituted SO₂-alkyl (e.g., SO₂—CH₃), C₁-C₅ substituted or unsubstituted C(O)—NH-alkyl (e.g., C(O)—NH—CH₃), C₁-C₅ linear or branched C(O)—O-alkyl (e.g., C(O)—O-tBu), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), or substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]q, [C]_{q}
wherein q is between 2 and 10;

R₁₀ and R₁₁ are each independently H, OH, substituted or unsubstituted C₁-C₅ linear or branched alkyl (e.g., methyl, ethyl, CH₂—CH₂—O—CH₃), C₁-C₅ linear or branched alkoxy (e.g., O—CH₃), substituted or unsubstituted C₃-C₈ heterocyclic ring (e.g., 1-(methylsulfonyl)piperidine, 1-(methylsulfonyl)piperazine, tetrahydro-2H-pyrane, morpholine, thiomorpholine 1,1-dioxide, methyl-pyrrolidine, methyl-piperidine), C(O)-alkyl, or S(O)₂-alkyl;

or R₁₀ and R₁₁ are joined to form a substituted or unsubstituted C₃-C₈ heterocyclic ring (e.g., morpholine, piperazine, piperidine, pyrrolidine, 1-methylpyrrolidin-2-one, oxetane, azetidine, 1-methylazetidine),

R₂₀ is represented by the following structure:

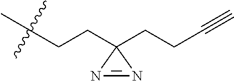 [SMILES: *CCC1(CCC#C)N=N1]

wherein substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof;

n and l are each independently an integer between 1 and 3 (e.g., 1 or 2);

m and k are each independently an integer between 0 and 3 (e.g., 0);

or its pharmaceutically acceptable salt, stereoisomer, tautomer, hydrate, N-oxide, prodrug, isotopic variant (e.g., deuterated analog), PROTAC, reverse amide, pharmaceutical product or any combination thereof.

In various embodiments, this invention is directed to a compound represented by the structure of formula IV:

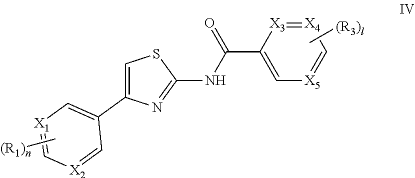

wherein

R₁ is H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl), or substituted or unsubstituted benzyl;

R₃ is H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)₂₀—CH₃), C(O)N(R₁₀)(R₁₁) (e.g., C(O)-piperidine, C(O)-pyrrolidine, C(O)N(CH₃)₂, C(O)-piperazine), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, N-methylpiperazine-1-carboxamide piperidin-4-ol, piperidin-3-ol, morpholine, 3-methylmorpholine, 3-hydroxypiperidine, tetrahydro-2H-pyrane, tetrahydro-2H-thiopyran 1,1-dioxide, pyrazole, thiazole, imidazole, pyrrolidine, pyrrolidinone, octahydropyrrolo[1,2-a]pyrazine, 6-methyl-2,6-diazaspiro[3.3]heptane, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl), or substituted or unsubstituted benzyl;

X₁, X₂ X₃, X₄ and X₅ are each independently C or N;

R is H, OH, F, Cl, Br, I, CN, CF₃, NO₂, NH₂, NH(R₁₀) (e.g., NH(CH₃)), N(R₁₀)(R₁₁), R₂₀, C₁-C₅ linear or branched, C₁-C₅ substituted or unsubstituted alkyl (e.g., methyl, ethyl, CH₂CH₂OH, CH₂CH₂OCH₃), R₈—R₁₀ (e.g., CH₂—OH, CH₂CH₂—OH), C(O)—R₁₀ (e.g., C(O)-methylpyrroldine, C(O)-methylpiperidine, C(O)—CH₃), C₁-C₅ substituted or unsubstituted C(O)-alkyl (e.g., C(O)—CH₂CH₂—OCH₃, C(O)—CH₃, C(O)—CH₂—N(CH₃)₂, C(O)—CH₂—CH₂—N(CH₃)₂, C(O)—CH₂—OH), C(O)—R₈—R₁₀ (e.g., C(O)—CH₂CH₂—OH), C(O)-substituted or unsubstituted C₃-C₈ heterocyclic ring (e.g., C(O)-methylpyrroldine, C(O)-methylpiperidine), C₁-C₅ substituted or unsubstituted SO₂-alkyl (e.g., SO₂—CH₃), C₁-C₅ substituted or unsubstituted C(O)—NH-alkyl (e.g., C(O)—NH—CH₃), C₁-C₅ linear or branched C(O)—O-alkyl (e.g., C(O)—O-tBu), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), or substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]q, [C]_{q}
wherein q is between 2 and 10;

R₁₀ and R₁₁ are each independently H, OH, substituted or unsubstituted C₁-C₅ linear or branched alkyl (e.g., methyl, ethyl, CH₂—CH₂—O—CH₃), C₁-C₅ linear or branched alkoxy (e.g., O—CH₃), substituted or unsubstituted C₃-C₈ heterocyclic ring (e.g., 1-(methylsulfonyl)piperidine, 1-(methylsulfonyl)piperazine, tetrahydro-2H-pyrane, morpholine, thiomorpholine 1,1-dioxide, methyl-pyrrolidine, methyl-piperidine), C(O)-alkyl, or S(O)₂-alkyl;

or R₁₀ and R₁₁ are joined to form a substituted or unsubstituted C₃-C₈ heterocyclic ring (e.g., morpholine, piperazine, piperidine, pyrrolidine, 1-methylpyrrolidin-2-one, oxetane, azetidine, 1-methylazetidine),

R₂₀ is represented by the following structure:

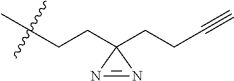 [SMILES: *CCC1(CCC#C)N=N1]

wherein substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof;

n and l are each independently an integer between 1 and 3 (e.g., 1 or 2);

or its pharmaceutically acceptable salt, stereoisomer, tautomer, hydrate, N-oxide, prodrug, isotopic variant (e.g., deuterated analog), PROTAC, reverse amide, pharmaceutical product or any combination thereof.

In various embodiments, this invention is directed to a compound represented by the structure of formula V:

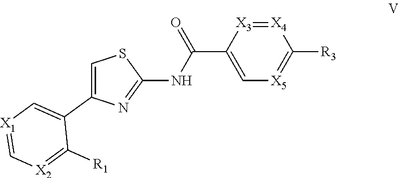

wherein

R₁ is H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl), or substituted or unsubstituted benzyl;

R₃ is H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)₂₀—CH₃), C(O)N(R₁₀)(R₁₁) (e.g., C(O)-piperidine, C(O)-pyrrolidine, C(O)N(CH₃)₂, C(O)-piperazine), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, N-methylpiperazine-1-carboxamide piperidin-4-ol, piperidin-3-ol, morpholine, 3-methylmorpholine, 3-hydroxypiperidine, tetrahydro-2H-pyrane, tetrahydro-2H-thiopyran 1,1-dioxide, pyrazole, thiazole, imidazole, pyrrolidine, pyrrolidinone, octahydropyrrolo[1,2-a]pyrazine, 6-methyl-2,6-diazaspiro[3.3]heptane, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl), or substituted or unsubstituted benzyl;

X₁, X₂ X₃, X₄ and X₅ are each independently C or N;

R is H, OH, F, Cl, Br, I, CN, CF₃, NO₂, NH₂, NH(R₁₀) (e.g., NH(CH₃)), N(R₁₀)(R₁₁), R₂₀, C₁-C₅ linear or branched, C₁-C₅ substituted or unsubstituted alkyl (e.g., methyl, ethyl, CH₂CH₂OH, CH₂CH₂OCH₃), R₈—R₁₀ (e.g., CH₂—OH, CH₂CH₂—OH), C(O)—R₁₀ (e.g., C(O)-methylpyrroldine, C(O)-methylpiperidine, C(O)—CH₃), C₁-C₅ substituted or unsubstituted C(O)-alkyl (e.g., C(O)—CH₂CH₂—OCH₃, C(O)—CH₃, C(O)—CH₂—N(CH₃)₂, C(O)—CH₂—CH₂—N(CH₃)₂, C(O)—CH₂—OH), C(O)—R₈—R₁₀ (e.g., C(O)—CH₂CH₂—OH), C(O)-substituted or unsubstituted C₃-C₈ heterocyclic ring (e.g., C(O)-methylpyrroldine, C(O)-methylpiperidine), C₁-C₅ substituted or unsubstituted SO₂-alkyl (e.g., SO₂—CH₃), C₁-C₅ substituted or unsubstituted C(O)—NH-alkyl (e.g., C(O)—NH—CH₃), C₁-C₅ linear or branched C(O)—O-alkyl (e.g., C(O)—O-tBu), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]q, [C]_{q}
wherein q is between 2 and 10;

R₁₀ and R₁₁ are each independently H, OH, substituted or unsubstituted C₁-C₅ linear or branched alkyl (e.g., methyl, ethyl, CH₂—CH₂—O—CH₃), C₁-C₅ linear or branched alkoxy (e.g., 0-CH₃), substituted or unsubstituted C₃-C₈ heterocyclic ring (e.g., 1-(methylsulfonyl)piperidine, 1-(methylsulfonyl)piperazine, tetrahydro-2H-pyrane, morpholine, thiomorpholine 1,1-dioxide, methyl-pyrrolidine, methyl-piperidine), C(O)-alkyl, or S(O)₂-alkyl;

or R₁₀ and R₁₁ are joined to form a substituted or unsubstituted C₃-C₈ heterocyclic ring (e.g., morpholine, piperazine, piperidine, pyrrolidine, 1-methylpyrrolidin-2-one, oxetane, azetidine, 1-methylazetidine),

R₂₀ is represented by the following structure:

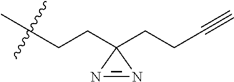 [SMILES: *CCC1(CCC#C)N=N1]

wherein substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof;

or its pharmaceutically acceptable salt, stereoisomer, tautomer, hydrate, N-oxide, prodrug, isotopic variant (e.g., deuterated analog), PROTAC, reverse amide, pharmaceutical product or any combination thereof.

In various embodiments, this invention is directed to a compound represented by the structure of formula VI:

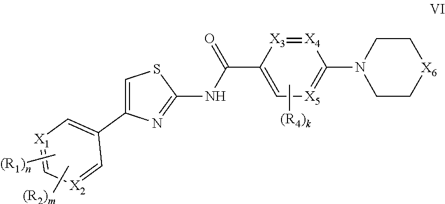

wherein

R₁ and R₂ are each independently H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl), substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., 1,4-dioxane, 2,3-dihydro-1,4-dioxine, dioxol, dioxolpyridine) ring;

R₄ is H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)₂₀—CH₃), C(O)N(R₁₀)(R₁₁) (e.g., C(O)-piperidine, C(O)-pyrrolidine, C(O)N(CH₃)₂, C(O)-piperazine), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, piperidin-4-ol, morpholine, tetrahydro-2H-pyrane, tetrahydro-2H-thiopyran 1,1-dioxide, pyrazole, thiazole, imidazole, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl), or substituted or unsubstituted benzyl;

X₁, X₂ X₃, X₄ and X₅ are each independently C or N;

X₆ is O, CH₂, CHR (e.g., CH(OH), CH(NH₂), CH(NH(CH₃))), C(R₁₀)(R₁₁) (e.g., C(H)CH₂CH₂—OH, C(H)CH₂—OH, 1-methylazetidine), NH, N—R (e.g., N—CH₃, N—SO₂—CH₃, N—R₂₀, N—CH₂CH₂—OCH₃) or N—C(O)—R₁₀ (e.g., N—C(O)O-tBu, N—C(O)—CH₂CH₂—OCH₃, N—C(O)—CH₃, N—C(O)—CH₂—N(CH₃)₂, N—C(O)—CH₂—CH₂—N(CH₃)₂, N—C(O)—CH₂—OH, N—C(O)—CH₂CH₂—OH, N—C(O)—NH—CH₃, N—C(O)-1-methyl-2-pyrrolidine, N—C(O)-1-methyl-3-pyrrolidine, N—C(O)-1-methyl-3-piperidine, N—C(O)-1-methyl-4-piperidine);

R is H, OH, F, Cl, Br, I, CN, CF₃, NO₂, NH₂, NH(R₁₀) (e.g., NH(CH₃)), N(R₁₀)(R₁₁), R₂₀, C₁-C₅ linear or branched, C₁-C₅ substituted or unsubstituted alkyl (e.g., methyl, ethyl, CH₂CH₂OH, CH₂CH₂OCH₃), R₈—R₁₀ (e.g., CH₂—OH, CH₂CH₂—OH), C(O)—R₁₀ (e.g., C(O)-methylpyrroldine, C(O)-methylpiperidine, C(O)—CH₃), C₁-C₅ substituted or unsubstituted C(O)-alkyl (e.g., C(O)—CH₂CH₂—OCH₃, C(O)—CH₃, C(O)—CH₂—N(CH₃)₂, C(O)—CH₂—CH₂—N(CH₃)₂, C(O)—CH₂—OH), C(O)—R₈—R₁₀ (e.g., C(O)—CH₂CH₂—OH), C(O)-substituted or unsubstituted C₃-C₈ heterocyclic ring (e.g., C(O)-methylpyrroldine, C(O)-methylpiperidine), C₁-C₅ substituted or unsubstituted SO₂-alkyl (e.g., SO₂—CH₃), C₁-C₅ substituted or unsubstituted C(O)—NH-alkyl (e.g., C(O)—NH—CH₃), C₁-C₅ linear or branched C(O)—O-alkyl (e.g., C(O)—O-tBu), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), or substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]q, [C]_{q}
wherein q is between 2 and 10;

R₁₀ and R₁₁ are each independently H, OH, substituted or unsubstituted C₁-C₅ linear or branched alkyl (e.g., methyl, ethyl, CH₂—CH₂—O—CH₃), C₁-C₅ linear or branched alkoxy (e.g., O—CH₃), substituted or unsubstituted C₃-C₈ heterocyclic ring (e.g., 1-(methylsulfonyl)piperidine, 1-(methylsulfonyl)piperazine, tetrahydro-2H-pyrane, morpholine, thiomorpholine 1,1-dioxide, methyl-pyrrolidine, methyl-piperidine), C(O)-alkyl, or S(O)₂-alkyl;

or R₁₀ and R₁₁ are joined to form a substituted or unsubstituted C₃-C₈ heterocyclic ring (e.g., morpholine, piperazine, piperidine, pyrrolidine, 1-methylpyrrolidin-2-one, oxetane, azetidine, 1-methylazetidine),

R₂₀ is represented by the following structure:

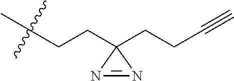 [SMILES: *CCC1(CCC#C)N=N1]

wherein substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof;

n is an integer between 1 and 3 (e.g., 1 or 2);

m and k are each independently an integer between 0 and 2 (e.g., 0);

or its pharmaceutically acceptable salt, stereoisomer, tautomer, hydrate, N-oxide, prodrug, isotopic variant (e.g., deuterated analog), PROTAC, reverse amide, pharmaceutical product or any combination thereof.

In various embodiments, this invention is directed to a compound represented by the structure of formula VII:

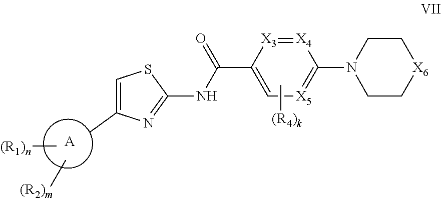

wherein

A ring is single or fused aromatic or heteroaromatic ring system (e.g., phenyl, thiophene, imidazole, pyrazole, pyrimidine, 2-, 3- or 4-pyridine, benzimidazole, indole, benzothiazole, benzooxazole, imidazopyridin, pyrazolopyridine, pyrrolopyridine, pyridazine, or pyrazine), or a single or fused C₃-C₁₀ cycloalkyl (e.g. pyrrolidin-2-one) or a single or fused C₃-C₁₀ heterocyclic ring (e.g., morpholine, piperidine, piperazine, tetrahydro-2H-pyran, azetidine, pyrrolidin-2-one);

R₁ and R₂ are each independently H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl), or substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., 1,4-dioxane, 2,3-dihydro-1,4-dioxine, dioxol, dioxolpyridine) ring;

R₄ is H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)₂₀—CH₃), C(O)N(R₁₀)(R₁₁) (e.g., C(O)-piperidine, C(O)-pyrrolidine, C(O)N(CH₃)₂, C(O)-piperazine), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, piperidin-4-ol, morpholine, tetrahydro-2H-pyrane, tetrahydro-2H-thiopyran 1,1-dioxide, pyrazole, thiazole, imidazole, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl), or substituted or unsubstituted benzyl;

X₃, X₄ and X₅ are each independently C or N;

X₆ is O, CH₂, CHR (e.g., CH(OH), CH(NH₂), CH(NH(CH₃))), C(R₁₀)(R₁₁) (e.g., C(H)CH₂CH₂—OH, C(H)CH₂—OH, 1-methylazetidine), NH, N—R (e.g., N—CH₃, N—SO₂—CH₃, N—R₂₀, N—CH₂CH₂—OCH₃) or N—C(O)—R₁₀ (e.g., N—C(O)O-tBu, N—C(O)—CH₂CH₂—OCH₃, N—C(O)—CH₃, N—C(O)—CH₂—N(CH₃)₂, N—C(O)—CH₂—CH₂—N(CH₃)₂, N—C(O)—CH₂—OH, N—C(O)—CH₂CH₂—OH, N—C(O)—NH—CH₃, N—C(O)-1-methyl-2-pyrrolidine, N—C(O)-1-methyl-3-pyrrolidine, N—C(O)-1-methyl-3-piperidine, N—C(O)-1-methyl-4-piperidine);

R is H, OH, F, Cl, Br, I, CN, CF₃, NO₂, NH₂, NH(R₁₀) (e.g., NH(CH₃)), N(R₁₀)(R₁₁), R₂₀, C₁-C₅ linear or branched, C₁-C₅ substituted or unsubstituted alkyl (e.g., methyl, ethyl, CH₂CH₂OH, CH₂CH₂OCH₃), R₈—R₁₀ (e.g., CH₂—OH, CH₂CH₂—OH), C(O)—R₁₀ (e.g., C(O)-methylpyrroldine, C(O)-methylpiperidine, C(O)—CH₃), C₁-C₅ substituted or unsubstituted C(O)-alkyl (e.g., C(O)—CH₂CH₂—OCH₃, C(O)—CH₃, C(O)—CH₂—N(CH₃)₂, C(O)—CH₂—CH₂—N(CH₃)₂, C(O)—CH₂—OH), C(O)—R₈—R₁₀ (e.g., C(O)—CH₂CH₂—OH), C(O)-substituted or unsubstituted C₃-C₈ heterocyclic ring (e.g., C(O)-methylpyrroldine, C(O)-methylpiperidine), C₁-C₅ substituted or unsubstituted SO₂-alkyl (e.g., SO₂—CH₃), C₁-C₅ substituted or unsubstituted C(O)—NH-alkyl (e.g., C(O)—NH—CH₃), C₁-C₅ linear or branched C(O)—O-alkyl (e.g., C(O)—O-tBu), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), or substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, OH, substituted or unsubstituted C₁-C₅ linear or branched alkyl (e.g., methyl, ethyl, CH₂—CH₂—O—CH₃), C₁-C₅ linear or branched alkoxy (e.g., O—CH₃), substituted or unsubstituted C₃-C₈ heterocyclic ring (e.g., 1-(methylsulfonyl)piperidine, 1-(methylsulfonyl)piperazine, tetrahydro-2H-pyrane, morpholine, thiomorpholine 1,1-dioxide, methyl-pyrrolidine, methyl-piperidine), C(O)-alkyl, or S(O)₂-alkyl;

or R₁₀ and R₁₁ are joined to form a substituted or unsubstituted C₃-C₈ heterocyclic ring (e.g., morpholine, piperazine, piperidine, pyrrolidine, 1-methylpyrrolidin-2-one, oxetane, azetidine, 1-methylazetidine),

R₂₀ is represented by the following structure:

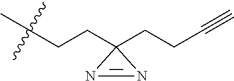 [SMILES: *CCC1(CCC#C)N=N1]

wherein substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof;

n is an integer between 1 and 3 (e.g., 1 or 2);

m and k are each independently an integer between 0 and 2 (e.g., 0);

or its pharmaceutically acceptable salt, stereoisomer, tautomer, hydrate, N-oxide, prodrug, isotopic variant (e.g., deuterated analog), PROTAC, reverse amide, pharmaceutical product or any combination thereof.

In various embodiments, this invention is directed to a compound represented by the structure of formula VIII

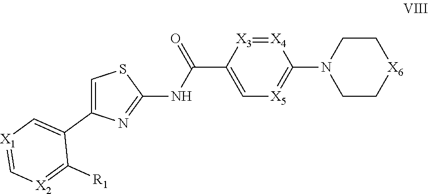

wherein

R₁ is H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl), substituted or unsubstituted benzyl;

X₁, X₂ X₃, X₄ and X₅ are each independently C or N;

X₆ is O, CH₂, CHR (e.g., CH(OH), CH(NH₂), CH(NH(CH₃))), C(R₁₀)(R₁₁) (e.g., C(H)CH₂CH₂—OH, C(H)CH₂—OH, 1-methylazetidine), NH, N—R (e.g., N—CH₃, N—SO₂—CH₃, N—R₂₀, N—CH₂CH₂—OCH₃) or N—C(O)—R₁₀ (e.g., N—C(O)O-tBu, N—C(O)—CH₂CH₂—OCH₃, N—C(O)—CH₃, N—C(O)—CH₂—N(CH₃)₂, N—C(O)—CH₂—CH₂—N(CH₃)₂, N—C(O)—CH₂—OH, N—C(O)—CH₂CH₂—OH, N—C(O)—NH—CH₃, N—C(O)-1-methyl-2-pyrrolidine, N—C(O)-1-methyl-3-pyrrolidine, N—C(O)-1-methyl-3-piperidine, N—C(O)-1-methyl-4-piperidine);

R is H, OH, F, Cl, Br, I, CN, CF₃, NO₂, NH₂, NH(R₁₀) (e.g., NH(CH₃)), N(R₁₀)(R₁₁), R₂₀, C₁-C₅ linear or branched, C₁-C₅ substituted or unsubstituted alkyl (e.g., methyl, ethyl, CH₂CH₂OH, CH₂CH₂OCH₃), R₈—R₁₀ (e.g., CH₂—OH, CH₂CH₂—OH), C(O)—R₁₀ (e.g., C(O)-methylpyrroldine, C(O)-methylpiperidine, C(O)—CH₃), C₁-C₅ substituted or unsubstituted C(O)-alkyl (e.g., C(O)—CH₂CH₂—OCH₃, C(O)—CH₃, C(O)—CH₂—N(CH₃)₂, C(O)—CH₂—CH₂—N(CH₃)₂, C(O)—CH₂—OH), C(O)—R₈—R₁₀ (e.g., C(O)—CH₂CH₂—OH), C(O)-substituted or unsubstituted C₃-C₈ heterocyclic ring (e.g., C(O)-methylpyrroldine, C(O)-methylpiperidine), C₁-C₅ substituted or unsubstituted SO₂-alkyl (e.g., SO₂—CH₃), C₁-C₅ substituted or unsubstituted C(O)—NH-alkyl (e.g., C(O)—NH—CH₃), C₁-C₅ linear or branched C(O)—O-alkyl (e.g., C(O)—O-tBu), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), or substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]q, [C]_{q}
wherein q is between 2 and 10;

R₁₀ and R₁₁ are each independently H, OH, substituted or unsubstituted C₁-C₅ linear or branched alkyl (e.g., methyl, ethyl, CH₂—CH₂—O—CH₃), C₁-C₅ linear or branched alkoxy (e.g., O—CH₃), substituted or unsubstituted C₃-C₈ heterocyclic ring (e.g., 1-(methylsulfonyl)piperidine, 1-(methylsulfonyl)piperazine, tetrahydro-2H-pyrane, morpholine, thiomorpholine 1,1-dioxide, methyl-pyrrolidine, methyl-piperidine), C(O)-alkyl, or S(O)₂-alkyl;

or R₁₀ and R₁₁ are joined to form a substituted or unsubstituted C₃-C₈ heterocyclic ring (e.g., morpholine, piperazine, piperidine, pyrrolidine, 1-methylpyrrolidin-2-one, oxetane, azetidine, 1-methylazetidine),

R₂₀ is represented by the following structure:

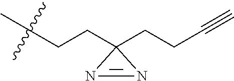 [SMILES: *CCC1(CCC#C)N=N1]

wherein substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof; or its pharmaceutically acceptable salt, stereoisomer, tautomer, hydrate, N-oxide, prodrug, isotopic variant (e.g., deuterated analog), PROTAC, reverse amide, pharmaceutical product or any combination thereof.

In some embodiments, at least one of R₁ and R₃ of compound of formula I-V is not H. In some embodiments, both R₁ and R₃ of compound of formula I-V are not H.

In some embodiments, R₁ of compound of formula I-VIII is Cl. In some embodiments, R₁ of compound of formula I-VIII is in the ortho position.

In some embodiments, R₃ is a substituted or unsubstituted, single, spirocyclic, fused, or bridged C₃-C₁₀ heterocycle. In some embodiments, R₃ is a morpholine, 3-methylmorpholine, 3-hydroxypiperidine, pyrrolidine, pyrrolidinone, octahydropyrrolo[1,2-a]pyrazine, or 6-methyl-2,6-diazaspiro[3.3]heptane; each represents a separate embodiment according to this invention. In some embodiments, R₃ is N(R₁₀)(R₁₁). In some embodiments, R₁ is Cl and R₃ is N(R₁₀)(R₁₁). In some embodiments, N(R₁₀)(R₁₁) is a substituted or unsubstituted C₃-C₈ heterocycle. In some embodiments, N(R₁₀)(R₁₁) is a substituted or unsubstituted 6-membered ring heterocycle. In some embodiments, N(R₁₀)(R₁₁) is morpholine, alkyl substituted morpholine, pyrrolidine, pyrrolidinone, piperazine, alkyl substituted piperazine (e.g., 1-(2-methoxyethyl)piperazine), amide substituted piperazine (e.g., N-methylpiperazine-1-carboxamide) sulphonyl substituted piperazine (e.g., 1- or 4-(methylsulfonyl)piperazine), octahydropyrrolo[1,2-a]pyrazine, hydroxy substituted piperidine, sulphonyl substituted piperidine (e.g., 1- or 4-(methylsulfonyl)piperidine), 2-methoxy-1-(piperazin-1-yl)ethenone, tetrahydro-2H-pyrane, tetrahydro-2H-thiopyran 1,1-dioxide, 6-methyl-2,6-diazaspiro[3.3]heptane; each is a separate embodiment according to this invention.

In some embodiments, if R₃ is a heterocycle, then R₁ cannot be H. In some embodiments, if R₃ is a heterocycle, then R₁ is Cl.

In some embodiments, at least one of X₃, X₄ and X₅ of formula II-VIII is N. In some embodiments, at least two of X₃, X₄ and X₅ is N.

In some embodiments, A of formula I, II, and/or VII is a phenyl. In other embodiments, A is pyridinyl. In other embodiments, A is 2-pyridinyl. In other embodiments, A is 3-pyridinyl. In other embodiments, A is 4-pyridinyl. In other embodiments, A is pyrimidine. In other embodiments, A is pyridazine. In other embodiments, A is pyrazine. In other embodiments, A is pyrazole. In other embodiments, A is naphthyl. In other embodiments, A is benzothiazolyl. In other embodiments, A is benzimidazolyl. In other embodiments, A is quinolinyl. In other embodiments, A is isoquinolinyl. In other embodiments, A is indolyl. In other embodiments, A is benzoxazole. In other embodiments, A is imidazopyridin. In other embodiments, A is pyrazolopyridine. In other embodiments, A is pyrrolopyridine. In other embodiments, A is tetrahydronaphthyl. In other embodiments, A is indenyl. In other embodiments, A is benzofuran-2(3H)-one. In other embodiments, A is benzo[d][1,3]dioxole. In other embodiments, A is tetrahydrothiophene1,1-dioxide. In other embodiments, A is thiazole. In others embodiment, A is piperidine. In other embodiments, A is tetrahydro-2H-pyran. In other embodiments, A is pyrrolidin-2-one. In other embodiments, A is morpholine. In other embodiments, A is piperazine. In other embodiments, A is azetidine. In other embodiments, A is 1-methylpiperidine. In other embodiments, A is imidazole. In other embodiments, A is 1-methylimidazole. In other embodiments, A is thiophene. In other embodiments, A is isoquinoline. In other embodiments, A is 1,3-dihydroisobenzofuran. In other embodiments, A is benzofuran. In other embodiments, A is single or fused C₃-C₁₀ cycloalkyl ring. In other embodiments, A is bicyclo[1.1.1]pentyl. In other embodiments, A is cyclobutyl. In other embodiments, A is cyclohexyl.

In some embodiments, B of formula I is a phenyl ring. In other embodiments, B is pyridinyl. In other embodiments, B is 2-pyridinyl. In other embodiments, B is 3-pyridinyl. In other embodiments, B is 4-pyridinyl. In other embodiments, B is pyrimidine. In other embodiments, B is pyridazine. In other embodiments, B is pyrazine. In other embodiments, B is piperidine. In other embodiments, B is, tetrahydro-2H-pyran. In other embodiments, B is azetidine. In other embodiments, B is thiazole. In other embodiments, B is imidazole. In other embodiments, B is indazole. In other embodiments, B is pyrrole. In other embodiments, B is naphthyl. In other embodiments, B is indolyl. In other embodiments, B is benzimidazolyl. In other embodiments, B is benzothiazolyl. In other embodiments, B is quinoxalinyl. In other embodiments, B is tetrahydronaphthyl. In other embodiments, B is quinolinyl. In other embodiments, B is isoquinolinyl. In other embodiments, B is indenyl. In other embodiments, B is naphthalene. In other embodiments, B is tetrahydrothiophene1,1-dioxide. In other embodiments, B is benzimidazole. In other embodiments, B is piperidine. In other embodiments, B is 1-methylpiperidine. In other embodiments, B is 1-methylimidazole. In other embodiments, B is thiophene. In other embodiments, B is isoquinoline. In other embodiments, B is indole. In other embodiments, B is 1,3-dihydroisobenzofuran. In other embodiments, B is benzofuran. In other embodiments, B is morpholine. In other embodiments, B is piperazine. In other embodiments, B is pyrrolidin-2-one. In other embodiments, B is single or fused C₃-C₁₀ cycloalkyl ring. In other embodiments, B is bicyclo[1.1.1]pentyl. In other embodiments, B is cyclobutyl. In other embodiments, B is cyclohexyl.

In some embodiments, X₁ of compound of formula III-VIII is N. In other embodiments, X₁ is C.

In some embodiments, X₂ of compound of formula III-VIII is N. In other embodiments, X₂ is C.

In some embodiments, X₃ of compound of formula II-VIII is N. In other embodiments, X₃ is C.

In some embodiments, X₄ of compound of formula II-VIII is N. In other embodiments, X₄ is C.

In some embodiments, X₅ of compound of formula II-VIII is N. In other embodiments, X₅ is C.

In some embodiments, X₆ of compound of formula VI-VIII is O. In other embodiments, X₆ is CHR. In other embodiments, X₆ is CH(OH). In other embodiments, X₆ is CH₂. In other embodiments, X₆ is CHR. In other embodiments, X₆ is CH(NH₂). In other embodiments, X₆ is CH(NH(CH₃))). In other embodiments, X₆ is C(H)CH₂—OH. In other embodiments, X₆ is 1-methylazetidine. In other embodiments, X₆ is N—R₂₀. In other embodiments, X₆ is C(R₁₀)(R₁₁). In other embodiments, X₆ is 1-methylpyrrolidin-2-one. In other embodiments, X₆ is oxetane. In other embodiments, X₆ is C(H)CH₂CH₂—OH. In other embodiments, X₆ is C(H)CH₂—OH. In other embodiments, X₆ is 1-methylazetidine. In other embodiments, X₆ is NH. In other embodiments, X₆ is N—R. In other embodiments, X₆ is N—CH₃. In other embodiments, X₆ is N—SO₂—CH₃. In other embodiments, X₆ is N—R₂₀. In other embodiments, X₆ is N—CH₂CH₂—OCH₃. In other embodiments, X₆ is N—C(O)O-tBu. In other embodiments, X₆ is N—C(O)—CH₂CH₂—OCH₃. In other embodiments, X₆ is N—CH₂CH₂—OCH₃. In other embodiments, X₆ is N—C(O)—R₁₀. In other embodiments, X₆ is N—C(O)—CH₃. In other embodiments, X₆ is C₁-C₅ substituted or unsubstituted N—C(O)—NH-alkyl. In other embodiments, X₆ is N—C(O)—NH—CH₃. In other embodiments, X₆ is N C(O)—CH₂—N(CH₃)₂. In other embodiments, X₆ is N—C(O)—CH₂—CH₂—N(CH₃)₂. In other embodiments, X₆ is N—C(O)—CH₂—OH. In other embodiments, X₆ is N—C(O)—CH₂CH₂—OH. In other embodiments, X₆ is N—C(O)—NH—CH₃. In other embodiments, X₆ is N—C(O)-1-methyl-2-pyrrolidine. In other embodiments, X₆ is N—C(O)-1-methyl-3-pyrrolidine. In other embodiments, X₆ is N—C(O)-1-methyl-3-piperidine. In other embodiments, X₆ is N—C(O)-1-methyl-4-piperidine. In other embodiments, X₆ is N—C(O)-1-methyl-3-piperidine.

It is understood that if any of X₁-X₅ are N, then any of R₁—R₄ cannot be attached thereto.

In some embodiments, R₁ of formula I-VIII is H. In some embodiments, R₁ is not H. In some embodiments, R₁ is Cl. In some embodiments, R₁ is F. In some embodiments, R₁ is R₈—OH. In some embodiments, R₁ is CH₂OH. In some embodiments, R₁ is —R₈—O—R₁₀. In some embodiments, R₁ is CH₂—O—CH₂—CH₂—O—CH₃. In some embodiments, R₁ is CH₂—O—CH₃. In some embodiments, R₁ is —O—R₈—O—R₁₀. In some embodiments, R₁ is O—CH₂—CH₂—O—CH₃. In some embodiments, R₁ is CN. In some embodiments, R₁ is R₈—N(R₁₀)(R₁₁). In some embodiments, R₁ is CH₂—NH—CH₃. In some embodiments, R₁ is CH₂—NH—C(O)CH₃. In some embodiments, R₁ is CH₂—N(CH₃)₂). In some embodiments, R₁ is alkyl. In some embodiments, R₁ is methyl. In some embodiments, R₁ is C₁-C₅ linear, branched or cyclic haloalkyl, C₁-C₅ linear, branched or cyclic alkoxy. In some embodiments, R₁ is methoxy. In some embodiments, R₁ is substituted or unsubstituted C₃-C₈ heterocyclic ring. In some embodiments, R₁ is azetidine. In some embodiments, R₁ is CF₃. In some embodiments, R₁ is CHF₂. In some embodiments, R₁ is C₁-C₅ linear or branched, substituted or unsubstituted alkyl. In other embodiments, R₁ is methyl. In other embodiments, R₁ is ethyl. In other embodiments, R₁ is iso-propyl. In other embodiments, R₁ is t-Bu. In other embodiments, R₁ is iso-butyl. In other embodiments, R₁ is pentyl. In other embodiments, R₁ is propyl. In other embodiments, R₁ is benzyl. In other embodiments, R₁ is in the ortho position. In other embodiments, R₁ is an ortho-methyl.

In some embodiments, R₂ of formula I-III, VI and/or VII is H. In some embodiments, R₂ is Cl. In some embodiments, R₂ is F. In some embodiments, R₂ is R₈—OH. In some embodiments, R₂ is CH₂OH. In some embodiments, R₂ is —R₈—O—R₁₀. In some embodiments, R₂ is CH₂—O—CH₂—CH₂—O—CH₃. In some embodiments, R₂ is CH₂—O—CH₃. In some embodiments, R₂ is —O—R₈—O—R₁₀. In some embodiments, R₂ is O—CH₂—CH₂—O—CH₃. In some embodiments, R₂ is CN. In some embodiments, R₂ is R₈—N(R₁₀)(R₁₁). In some embodiments, R₂ is CH₂—NH—CH₃. In some embodiments, R₂ is CH₂—NH—C(O)CH₃. In some embodiments, R₂ is CH₂—N(CH₃)₂). In some embodiments, R₂ is C₁-C₅ linear or branched, substituted or unsubstituted alkyl. In other embodiments, R₂ is methyl. In other embodiments, R₂ is ethyl. In other embodiments, R₂ is iso-propyl. In other embodiments, R₂ is t-Bu. In other embodiments, R₂ is iso-butyl. In other embodiments, R₂ is pentyl. In other embodiments, R₂ is propyl. In other embodiments, R₂ is benzyl. In other embodiments, R₂ is in the ortho position. In other embodiments, R₂ is an ortho-methyl. In other embodiments, R₂ is C₁-C₅ linear, branched or cyclic alkoxy. In other embodiments, R₂ is methoxy. In other embodiments, R₂ is ethoxy. In other embodiments, R₂ is propoxy. In other embodiments, R₂ is isopropoxy. In other embodiments, R₂ is substituted or unsubstituted aryl. In other embodiments, R₂ is phenyl. In other embodiments, substitutions include: C₁-C₅ linear or branched alkyl (e.g. methyl), aryl, phenyl, heteroaryl (e.g., imidazole), and/or C₃-C₈ cycloalkyl, each is a separate embodiment according to this invention.

In some embodiments, R₁ and R₂ of formula I-III, VI and/or VII are joined together to form a pyrrol ring. In some embodiments, R₁ and R₂ are joined together to form a 1,4-dioxane ring. In some embodiments, R₁ and R₂ are joined together to form a 2,3-dihydro-1,4-dioxine ring. In some embodiments, R₁ and R₂ are joined together to form a benzene ring. In some embodiments, R₁ and R₂ are joined together to form a pyridine ring. In some embodiments, R₁ and R₂ are joined together to form a furanone ring (e.g., furan-2(3H)-one).

In some embodiments, R₃ of formula I-V is H. In other embodiments, R₃ is F. In other embodiments, R₃ is Cl. In other embodiments, R₃ is Br. In other embodiments, R₃ is I. In other embodiments, R₃ is N(R₁₀)(R₁₁). In other embodiments, R₃ is morpholine. In other embodiments, R₃ is piperazine. In other embodiments, R₃ is C(O)—R₁₀. In other embodiments, R₃ is C(O)NHR. In other embodiments, R₃ is C(O)NH(CH₃)₂₀—CH₃. In other embodiments, R₃ is C(O)N(R₁₀)(R₁₁). In other embodiments, R₃ is C(O)-piperidine. In other embodiments, R₃ is C(O)-pyrrolidine. In other embodiments, R₃ is C(O)N(CH₃)₂). In other embodiments, R₃ is SO₂R. In other embodiments, R₃ is C₁-C₅ linear or branched, substituted or unsubstituted alkyl. In other embodiments, R₃ is methyl. In other embodiments, R₃ is ethyl. In other embodiments, R₃ is C₁-C₅ linear, branched or cyclic haloalkyl. In other embodiments, R₃ is CHF₂. In other embodiments, R₃ is C₁-C₅ linear, branched or cyclic alkoxy. In other embodiments, R₃ is methoxy. In other embodiments, R₃ is 1-(methylsulfonyl)piperidin-4-oxy. In other embodiments, R₃ is 1-(methyl)piperidin-4-oxy. In other embodiments, R₃ is 1-(ethanone)piperidin-4-oxy. In other embodiments, R₃ is substituted or unsubstituted C₃-C₈ cycloalkyl. In other embodiments, R₃ is substituted or unsubstituted, single, spirocyclic, fused, or bridged C₃-C₁₀ heterocyclic ring. In other embodiments, R₃ is piperazine. In other embodiments, R₃ is 1-(2-methoxyethyl)piperazine. In other embodiments, R₃ is 1- or 4-(methylsulfonyl)piperidine. In other embodiments, R₃ is 2-methoxy-1-(piperazin-1-yl)ethenone. In other embodiments, R₃ is morpholine. In other embodiments, R₃ is 3-methylmorpholine. In other embodiments, R₃ is 3-hydroxypiperidine. In other embodiments, R₃ is pyrrolidine. In other embodiments, R₃ is pyrrolidinone. In other embodiments, R₃ is octahydropyrrolo[1,2-a]pyrazine. In other embodiments, R₃ is 6-methyl-2,6-diazaspiro[3.3]heptane. In other embodiments, R₃ is tetrahydro-2H-thiopyran 1,1-dioxide. In other embodiments, R₃ is 1- or 4-methylpiperazine. In other embodiments, R₃ is 1- or 4-(methylsulfonyl)piperazine. In other embodiments, R₃ is 1-(piperazin-1-yl)ethanone. In other embodiments, R₃ is 2-(dimethylamino)-1-(piperazin-1-yl)ethanone. In other embodiments, R₃ is 2-(dimethylamino)-1-(piperazin-1-yl)propanone. In other embodiments, R₃ is 2-hydroxy-1-(piperazin-1-yl)ethenone. In other embodiments, R₃ is N-methylpiperazine-1-carboxamide. In other embodiments, R₃ is piperidin-4-ol. In other embodiments, R₃ is piperidin-3-ol. In other embodiments, R₃ is tetrahydro-2H-pyrane. In other embodiments, R₃ is 2-oxa-7-azaspiro[3.5]nonane. In other embodiments, R₃ is 1-(2,6-diazaspiro[3.3]heptan-2-yl)ethenone. In other embodiments, R₃ is 2-methoxy-1-(2,6-diazaspiro[3.3]heptan-2-yl)ethenone. In other embodiments, R₃ is 2,8-diazaspiro[4.5]decan-1-one. In other embodiments, R₃ is 2-methyl-2,8-diazaspiro[4.5]decan-1-one. In other embodiments, R₃ is 2-oxa-7-azaspiro[3.5]nonane. In other embodiments, R₃ is tetrahydro-2H-thiopyran 1,1-dioxide. In other embodiments, R₃ is pyrrolidine. In other embodiments, R₃ is (1-methylpiperidin-3-yl)(piperazin-1-yl)methanone. In other embodiments, R₃ may be further substituted with at least one substituent selected from: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl and (benzyloxy)phenyl.

In some embodiments, R₄ of formula I-III, and/or VI-VII is H. In other embodiments, R₄ is C₁-C₅ linear or branched, substituted or unsubstituted alkyl. In other embodiments, R₄ is methyl. In other embodiments, R₄ is ethyl.

In some embodiments, R₃ and R₄ of formula I-III are joined together to form a 5 or 6 membered substituted or unsubstituted, aliphatic ring. In some embodiments, R₃ and R₄ are joined together to form a cyclopentene. In some embodiments, R₃ and R₄ are joined together to form an aromatic carbocyclic ring. In some embodiments, R₃ and R₄ are joined together to form a benzene. In some embodiments, R₃ and R₄ are joined together to form an aromatic heterocyclic ring. In some embodiments, R₃ and R₄ are joined together to form a thiophene. In some embodiments, R₃ and R₄ are joined together to form a furane. In some embodiments, R₃ and R₄ are joined together to form a pyrrol. In some embodiments, R₃ and R₄ are joined together to form a pyrazole ring. a [1,3]dioxole ring. In some embodiments, R₃ and R₄ are joined together to form a furanone ring (e.g., furan-2(3H)-one). In some embodiments, R₃ and R₄ are joined together to form a cyclopentene ring. In some embodiments, R₃ and R₄ are joined together to form an imidazole ring.

In some embodiments, R₅ of formula I is H. In some embodiments, R₅ is R₂₀. In some embodiments, R₅ is C₁-C₅ linear or branched, substituted or unsubstituted alkyl. In some embodiments, R₅ is methyl. In some embodiments, R₅ is ethyl. In some embodiments, R₅ is C(O)—R₁₀. In some embodiments, R₅ is SO₂R.

In some embodiments, R of formula I-VIII is H. In other embodiments, R is OH. In other embodiments, R is NH₂. In other embodiments, R is NH(R₁₀). In other embodiments, R is NH(CH₃)). In other embodiments, R is R₂₀. In other embodiments, R is C₁-C₅ linear or branched, substituted or unsubstituted alkyl. In other embodiments, R is substituted alkyl. In other embodiments, R is methyl. In other embodiments, R is ethyl. In other embodiments, R is CH₂CH₂OCH₃. In other embodiments, R is CH₂CH₂OH. In other embodiments, R is R₈—R₁₀. In other embodiments, R is CH₂—OH. In other embodiments, R is CH₂CH₂—OH. In other embodiments, R is C(O)—R₁₀. In other embodiments, R is C(O)-methylpyrroldine. In other embodiments, R is C(O)-methylpiperidine. In other embodiments, R is C(O)—CH₃). In other embodiments, R is C₁-C₅ substituted or unsubstituted C(O)-alkyl. In other embodiments, R is C(O)—CH₂CH₂—OCH₃. In other embodiments, R is C(O)—CH₃. In other embodiments, R is C(O)—R₈—R₁₀. In other embodiments, R is C(O)—CH₂CH₂—OH. In other embodiments, R is C(O)-substituted or unsubstituted C₃-C₈ heterocyclic ring. In other embodiments, R is C(O)-methylpyrroldine. In other embodiments, R is C(O)-methylpiperidine. In other embodiments, R is C₁-C₅ substituted or unsubstituted SO₂-alkyl. In other embodiments, R is SO₂—CH₃. In other embodiments, R is —R₈—O—R₁₀. In other embodiments, R is CH₂—CH₂—O—CH₃. In other embodiments, R is C(O)—CH₂—N(CH₃)₂. In other embodiments, R is C(O)—CH₂—CH₂—N(CH₃)₂. In other embodiments, R is C(O)—CH₂—OH. In other embodiments, R is C₁-C₅ substituted or unsubstituted C(O)—NH-alkyl. In other embodiments, R is C(O)—NH—CH₃. In other embodiments, R is C₁-C₅ linear or branched C(O)—O-alkyl. In other embodiments, R is C(O)—O-tBu. In other embodiments, R may be further substituted with at least one substitution selected from: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl and (benzyloxy)phenyl; each represents a separate embodiment according to this invention. In some embodiment, two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring.

In some embodiments, R₈ of formula I-VIII is CH₂. In other embodiments, R₈ is CH₂CH₂. In other embodiments, R₈ is CH₂CH₂CH₂.

In some embodiments, p of formula I-VII is 1. In other embodiments, p is 2. In other embodiments, p is 3.

In some embodiments, R₉ of formula I-VIII is C≡C.

In some embodiments, q of formula I-VII is 2.

In some embodiments, R₁₀ of formula I-VIII is substituted or unsubstituted C₁-C₅ linear or branched alkyl. In other embodiments, R₁₀ is H. In other embodiments, R₁₀ is CH₃. In other embodiments, R₁₀ is CH₂CH₃. In other embodiments, R₁₀ is CH₂CH₂CH₃. In other embodiments, R₁₀ is CH₂—CH₂—O—CH₃. In other embodiments, R₁₀ is OH. In other embodiments, R₁₀ is substituted or unsubstituted C₃-C₈ heterocyclic ring. In other embodiments, R₁₀ is 1-(methylsulfonyl)piperidine. In other embodiments, R₁₀ is 1-(methylsulfonyl)piperazine. In other embodiments, R₁₀ is tetrahydro-2H-pyrane. In other embodiments, R₁₀ is morpholine. In other embodiments, R₁₀ is thiomorpholine 1,1-dioxide. In other embodiments, R₁₀ is methyl-pyrrolidine. In other embodiments, R₁₀ is methyl-piperidine.

In some embodiments, R₁ of formula I-VII is C₁-C₅ linear or branched alkyl. In other embodiments, R₁₀ is H. In other embodiments, R₁₁ is CH₃.

In some embodiments, R₁₀ and R₁₁ of formula I-VII are joined to form a substituted or unsubstituted C₃-C₈ heterocyclic ring. In other embodiments, R₁₀ and R₁₁ are joined to form a morpholine ring. In other embodiments, R₁₀ and R₁₁ are joined to form an unsubstituted piperazine ring. In other embodiments, R₁₀ and R₁₁ are joined to form a substituted piperazine ring. In other embodiments, R₁₀ and R₁₁ are joined to form an unsubstituted piperidine ring. In other embodiments, R₁₀ and R₁₁ are joined to form an unsubstituted pyrrolidine ring. In other embodiments, R₁₀ and R₁₁ are joined to form a substituted piperidine ring. In other embodiments, R₁₀ and R₁₁ are joined to form a 1-methylpyrrolidin-2-one ring. In other embodiments, R₁₀ and R₁₁ are joined to form an oxetane ring. In other embodiments, R₁₀ and R₁₁ are joined to form an azetidine ring. In other embodiments, R₁₀ and R₁₁ are joined to form an 1-methylazetidine. In some embodiments, substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof; each represents a separate embodiment according to this invention.

In some embodiments, n of formula I-IV and/or VI-VII is 1. In other embodiments, n is 2.

In some embodiments, m of formula I-III and/or VI-VII is 0. In some embodiments, m is 1. In some embodiments, m is 2.

In some embodiments, k of formula I-III and/or VI-VII is 0. In other embodiments, k is 1. In other embodiments, k is 2.

In some embodiments, 1 of formula I-IV is 1. In other embodiments, 1 is 2. In other embodiments, 1 is 3.

In some embodiments, Qi of formula I is S. In other embodiments, Qi is O. In other embodiments, Qi is NH.

In some embodiments, G=X of formula I is C═O. In other embodiments, G=X is C═S. In other embodiments, G=X is S═O. In other embodiments, G=X is SO₂.

In various embodiments, this invention is directed to the compounds presented in Table 1, pharmaceutical compositions and/or method of use thereof:

TABLE 1

| Compound | |
|---|---|
| Number | Compound Structure |
| 300 | 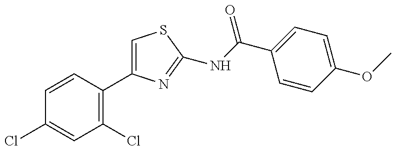 [SMILES: COc1ccc(C(=O)Nc2nc(-c3ccc(Cl)cc3Cl)cs2)cc1] |
| 301 | 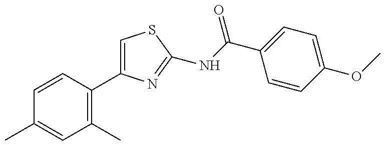 |
| 302 | 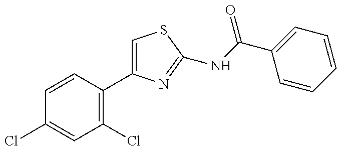 [SMILES: O=C(Nc1nc(-c2ccc(Cl)cc2Cl)cs1)c1ccccc1] |
| 303 | 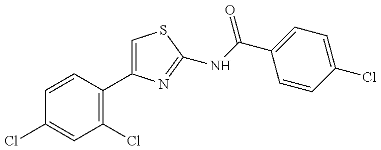 [SMILES: O=C(Nc1nc(-c2ccc(Cl)cc2Cl)cs1)c1ccc(Cl)cc1] |
| 304 | 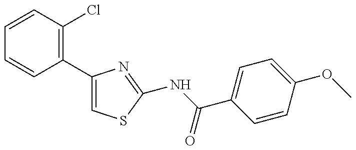 [SMILES: COc1ccc(C(=O)Nc2nc(-c3ccccc3Cl)cs2)cc1] |
| 305 | 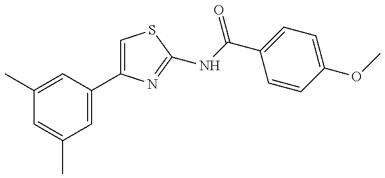 |
| 306 | 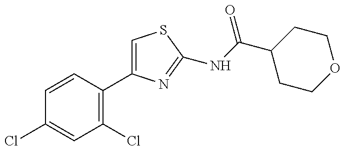 [SMILES: O=C(Nc1nc(-c2ccc(Cl)cc2Cl)cs1)C1CCOCC1] |
| 307 | 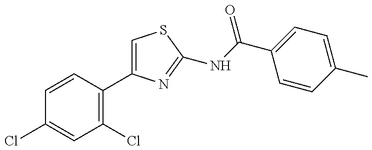 [SMILES: Cc1ccc(C(=O)Nc2nc(-c3ccc(Cl)cc3Cl)cs2)cc1] |
| 308 | 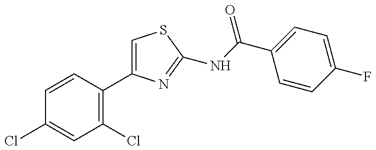 [SMILES: O=C(Nc1nc(-c2ccc(Cl)cc2Cl)cs1)c1ccc(F)cc1] |
| 310 | 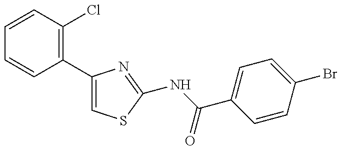 [SMILES: O=C(Nc1nc(-c2ccccc2Cl)cs1)c1ccc(Br)cc1] |
| 311 | 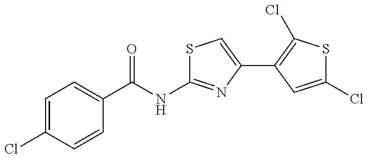 [SMILES: O=C(Nc1nc(-c2cc(Cl)sc2Cl)cs1)c1ccc(Cl)cc1] |
| 312 | 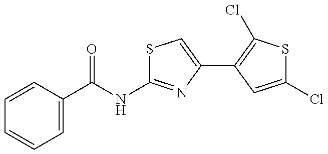 [SMILES: O=C(Nc1nc(-c2cc(Cl)sc2Cl)cs1)c1ccccc1] |
| 313 | 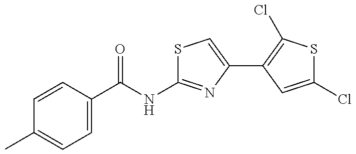 [SMILES: Cc1ccc(C(=O)Nc2nc(-c3cc(Cl)sc3Cl)cs2)cc1] |
| 314 | 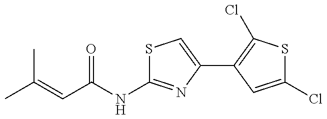 [SMILES: CC(C)=CC(=O)Nc1nc(-c2cc(Cl)sc2Cl)cs1] |
| 315 | 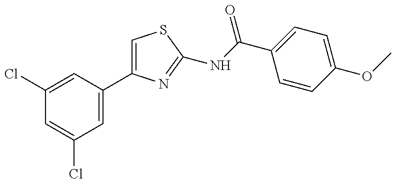 [SMILES: COc1ccc(C(=O)Nc2nc(-c3cc(Cl)cc(Cl)c3)cs2)cc1] |
| 316 | 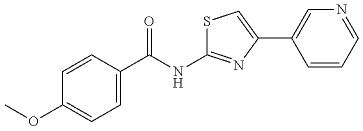 [SMILES: COc1ccc(C(=O)Nc2nc(-c3cccnc3)cs2)cc1] |
| 317 | 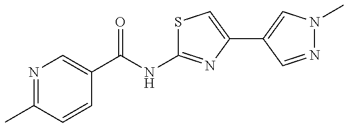 |
| 318 | 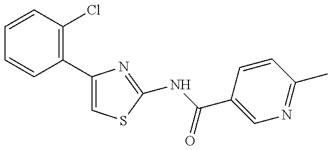 [SMILES: Cc1ccc(C(=O)Nc2nc(-c3ccccc3Cl)cs2)cn1] |
| 319 | 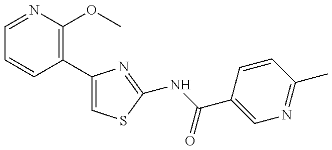 [SMILES: COc1ncccc1-c1csc(NC(=O)c2ccc(C)nc2)n1] |
| 320 | 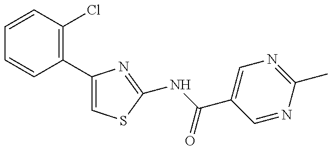 [SMILES: Cc1ncc(C(=O)Nc2nc(-c3ccccc3Cl)cs2)cn1] |
| 321 | 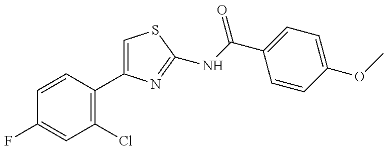 [SMILES: COc1ccc(C(=O)Nc2nc(-c3ccc(F)cc3Cl)cs2)cc1] |
| 322 | 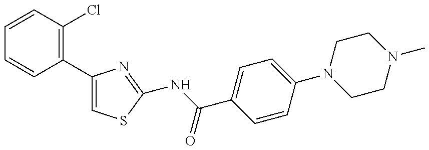 [SMILES: CN1CCN(c2ccc(C(=O)Nc3nc(-c4ccccc4Cl)cs3)cc2)CC1] |
| 323 | 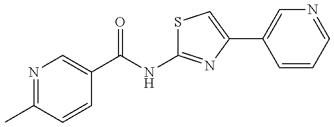 [SMILES: Cc1ccc(C(=O)Nc2nc(-c3cccnc3)cs2)cn1] |
| 324 | 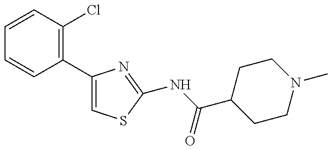 [SMILES: CN1CCC(C(=O)Nc2nc(-c3ccccc3Cl)cs2)CC1] |
| 325 | 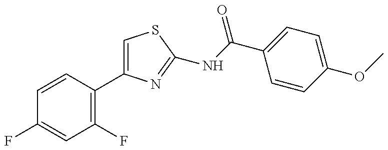 [SMILES: COc1ccc(C(=O)Nc2nc(-c3ccc(F)cc3F)cs2)cc1] |
| 326 | 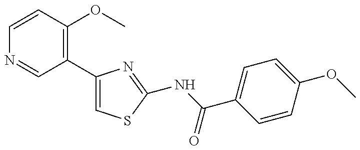 [SMILES: COc1ccc(C(=O)Nc2nc(-c3cnccc3OC)cs2)cc1] |
| 327 | 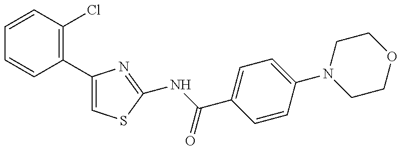 [SMILES: O=C(Nc1nc(-c2ccccc2Cl)cs1)c1ccc(N2CCOCC2)cc1] |
| 328 | 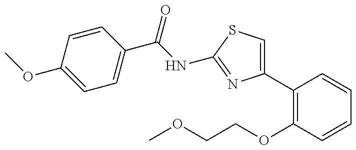 [SMILES: COCCOc1ccccc1-c1csc(NC(=O)c2ccc(OC)cc2)n1] |
| 329 | 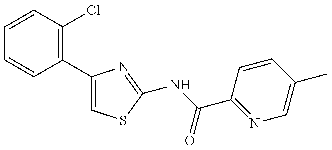 [SMILES: Cc1ccc(C(=O)Nc2nc(-c3ccccc3Cl)cs2)nc1] |
| 330 | 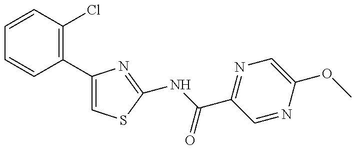 [SMILES: COc1cnc(C(=O)Nc2nc(-c3ccccc3Cl)cs2)cn1] |
| 331 | 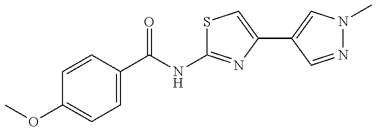 |
| 332 | 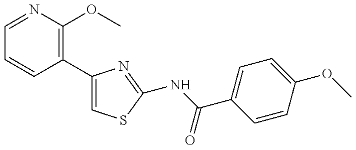 [SMILES: COc1ccc(C(=O)Nc2nc(-c3cccnc3OC)cs2)cc1] |
| 333 | 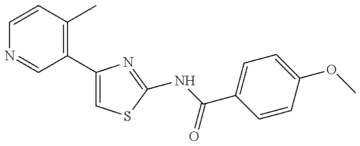 [SMILES: COc1ccc(C(=O)Nc2nc(-c3cnccc3C)cs2)cc1] |
| 334 | 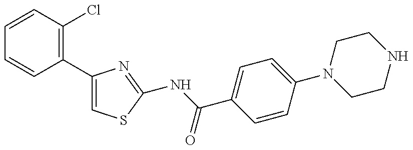 [SMILES: O=C(Nc1nc(-c2ccccc2Cl)cs1)c1ccc(N2CCNCC2)cc1] |
| 335 | 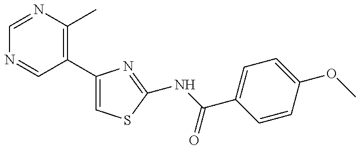 [SMILES: COc1ccc(C(=O)Nc2nc(-c3cncnc3C)cs2)cc1] |
| 336 | 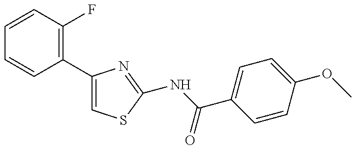 [SMILES: COc1ccc(C(=O)Nc2nc(-c3ccccc3F)cs2)cc1] |
| 337 | 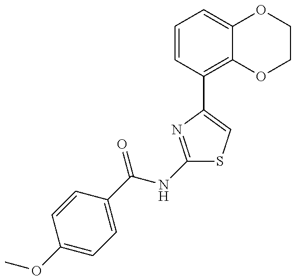 [SMILES: COc1ccc(C(=O)Nc2nc(-c3cccc4c3OCCO4)cs2)cc1] |
| 338 | 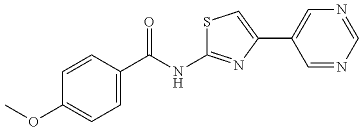 [SMILES: COc1ccc(C(=O)Nc2nc(-c3cncnc3)cs2)cc1] |
| 339 | 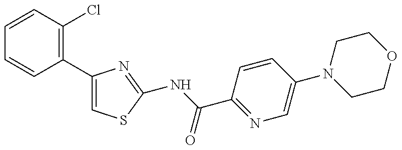 [SMILES: O=C(Nc1nc(-c2ccccc2Cl)cs1)c1ccc(N2CCOCC2)cn1] |
| 340 | 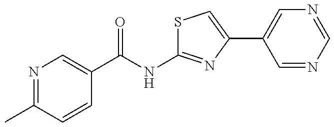 [SMILES: Cc1ccc(C(=O)Nc2nc(-c3cncnc3)cs2)cn1] |
| 341 | 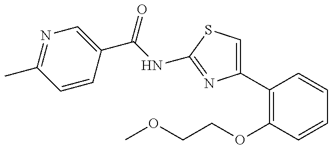 [SMILES: COCCOc1ccccc1-c1csc(NC(=O)c2ccc(C)nc2)n1] |
| 342 | 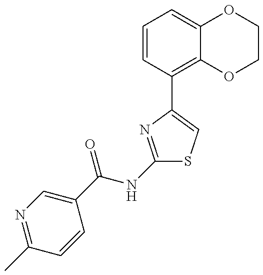 [SMILES: Cc1ccc(C(=O)Nc2nc(-c3cccc4c3OCCO4)cs2)cn1] |
| 343 | 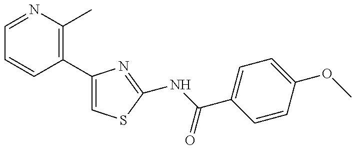 [SMILES: COc1ccc(C(=O)Nc2nc(-c3cccnc3C)cs2)cc1] |
| 344 | 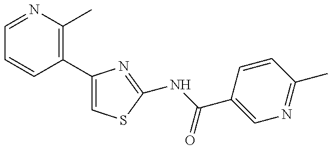 [SMILES: Cc1ccc(C(=O)Nc2nc(-c3cccnc3C)cs2)cn1] |
| 345 | 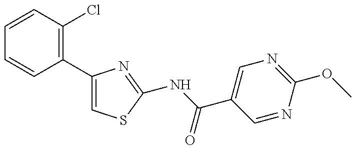 [SMILES: COc1ncc(C(=O)Nc2nc(-c3ccccc3Cl)cs2)cn1] |
| 346 | 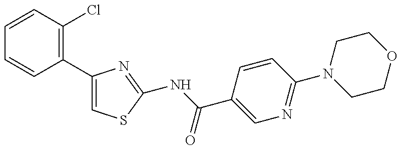 [SMILES: O=C(Nc1nc(-c2ccccc2Cl)cs1)c1ccc(N2CCOCC2)nc1] |
| 347 | 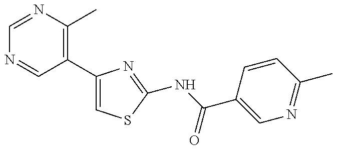 [SMILES: Cc1ccc(C(=O)Nc2nc(-c3cncnc3C)cs2)cn1] |
| 348 | 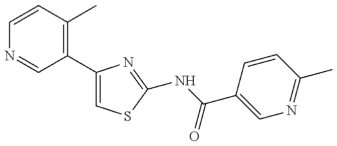 [SMILES: Cc1ccc(C(=O)Nc2nc(-c3cnccc3C)cs2)cn1] |
| 349 | 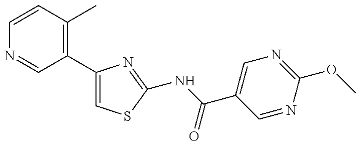 [SMILES: COc1ncc(C(=O)Nc2nc(-c3cnccc3C)cs2)cn1] |
| 350 | 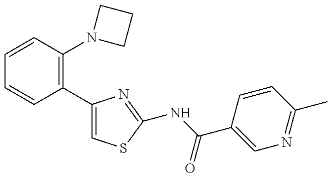 [SMILES: Cc1ccc(C(=O)Nc2nc(-c3ccccc3N3CCC3)cs2)cn1] |
| 351 | 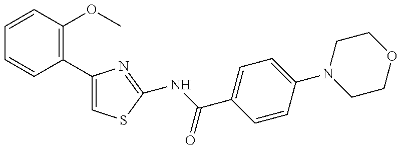 [SMILES: COc1ccccc1-c1csc(NC(=O)c2ccc(N3CCOCC3)cc2)n1] |
| 352 | 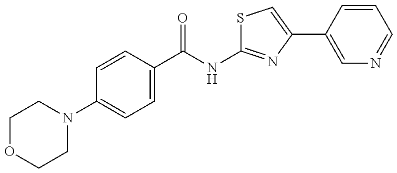 [SMILES: O=C(Nc1nc(-c2cccnc2)cs1)c1ccc(N2CCOCC2)cc1] |
| 353 | 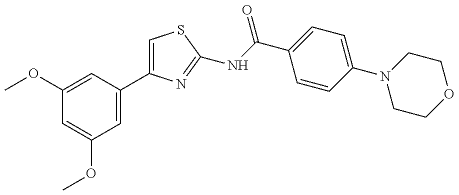 [SMILES: COc1cc(OC)cc(-c2csc(NC(=O)c3ccc(N4CCOCC4)cc3)n2)c1] |
| 354 | 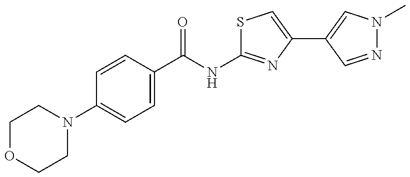 [SMILES: Cn1cc(-c2csc(NC(=O)c3ccc(N4CCOCC4)cc3)n2)cn1] |
| 355 | 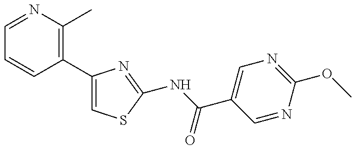 [SMILES: COc1ncc(C(=O)Nc2nc(-c3cccnc3C)cs2)cn1] |
| 356 | 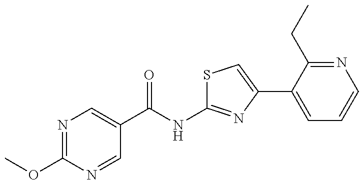 [SMILES: CCc1ncccc1-c1csc(NC(=O)c2cnc(OC)nc2)n1] |
| 357 | 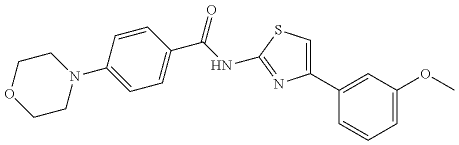 [SMILES: COc1cccc(-c2csc(NC(=O)c3ccc(N4CCOCC4)cc3)n2)c1] |
| 358 | 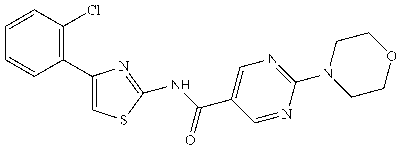 [SMILES: O=C(Nc1nc(-c2ccccc2Cl)cs1)c1cnc(N2CCOCC2)nc1] |
| 359 | 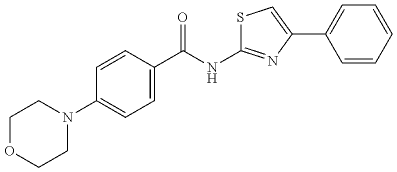 [SMILES: O=C(Nc1nc(-c2ccccc2)cs1)c1ccc(N2CCOCC2)cc1] |
| 360 | 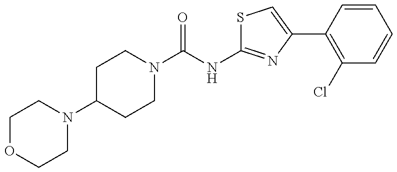 [SMILES: O=C(Nc1nc(-c2ccccc2Cl)cs1)N1CCC(N2CCOCC2)CC1] |
| 361 | 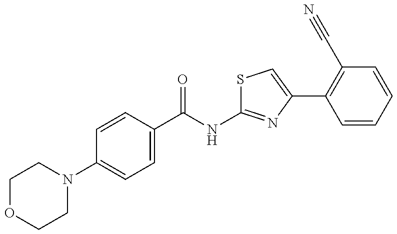 [SMILES: N#Cc1ccccc1-c1csc(NC(=O)c2ccc(N3CCOCC3)cc2)n1] |
| 363 | 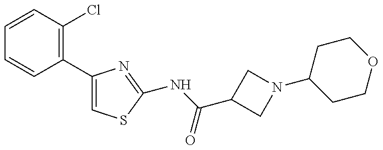 [SMILES: O=C(Nc1nc(-c2ccccc2Cl)cs1)C1CN(C2CCOCC2)C1] |
| 365 | 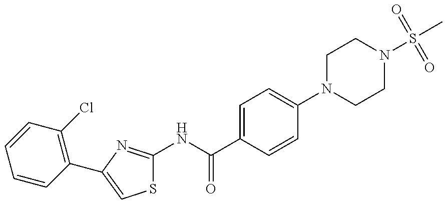 [SMILES: CS(=O)(=O)N1CCN(c2ccc(C(=O)Nc3nc(-c4ccccc4Cl)cs3)cc2)CC1] |
| 366 | 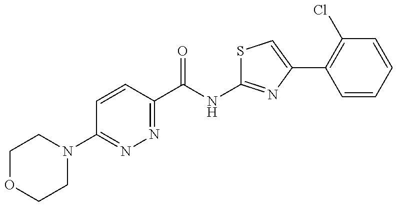 [SMILES: O=C(Nc1nc(-c2ccccc2Cl)cs1)c1ccc(N2CCOCC2)nn1] |
| 367 | 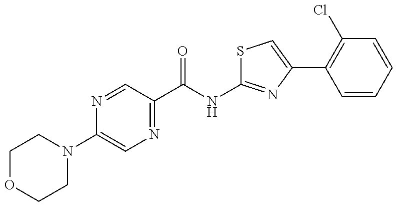 [SMILES: O=C(Nc1nc(-c2ccccc2Cl)cs1)c1cnc(N2CCOCC2)cn1] |
| 368 | 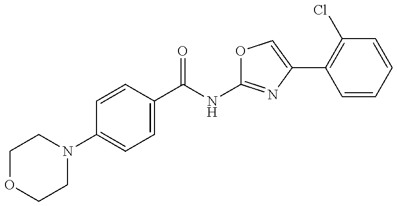 [SMILES: O=C(Nc1nc(-c2ccccc2Cl)co1)c1ccc(N2CCOCC2)cc1] |
| 369 | 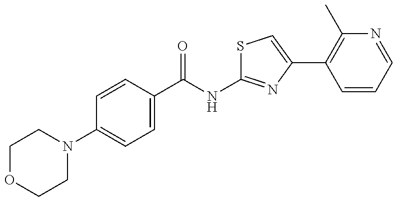 [SMILES: Cc1ncccc1-c1csc(NC(=O)c2ccc(N3CCOCC3)cc2)n1] |
| 370 | 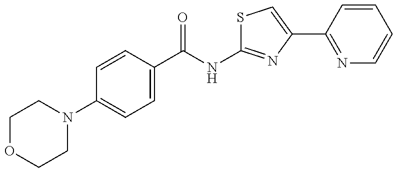 [SMILES: O=C(Nc1nc(-c2ccccn2)cs1)c1ccc(N2CCOCC2)cc1] |
| 371 | 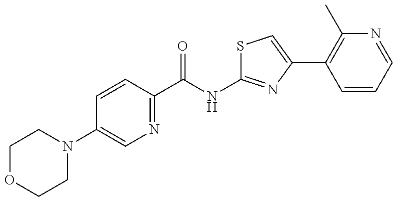 [SMILES: Cc1ncccc1-c1csc(NC(=O)c2ccc(N3CCOCC3)cn2)n1] |
| 372 | 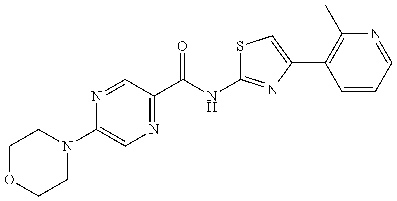 [SMILES: Cc1ncccc1-c1csc(NC(=O)c2cnc(N3CCOCC3)cn2)n1] |
| 373 | 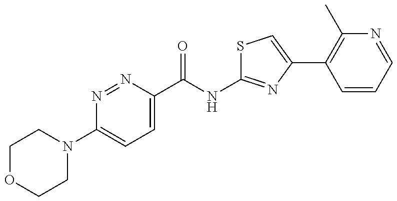 [SMILES: Cc1ncccc1-c1csc(NC(=O)c2ccc(N3CCOCC3)nn2)n1] |
| 374 | 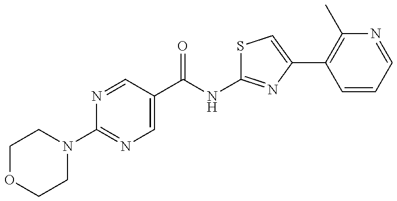 [SMILES: Cc1ncccc1-c1csc(NC(=O)c2cnc(N3CCOCC3)nc2)n1] |
| 375 | 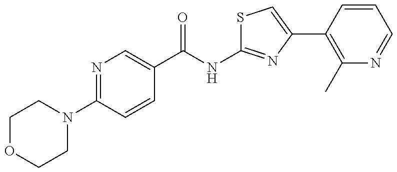 [SMILES: Cc1ncccc1-c1csc(NC(=O)c2ccc(N3CCOCC3)nc2)n1] |
| 376 | 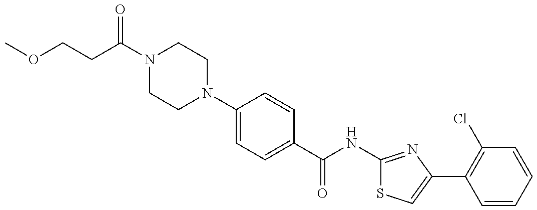 [SMILES: COCCC(=O)N1CCN(c2ccc(C(=O)Nc3nc(-c4ccccc4Cl)cs3)cc2)CC1] |
| 377 | 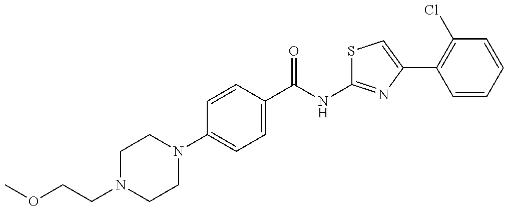 [SMILES: COCCN1CCN(c2ccc(C(=O)Nc3nc(-c4ccccc4Cl)cs3)cc2)CC1] |
| 378 | 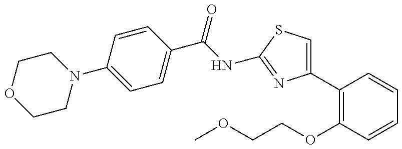 [SMILES: COCCOc1ccccc1-c1csc(NC(=O)c2ccc(N3CCOCC3)cc2)n1] |
| 379 | 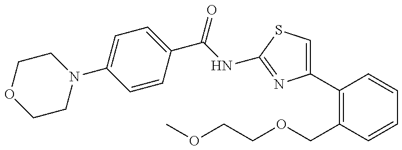 [SMILES: COCCOCc1ccccc1-c1csc(NC(=O)c2ccc(N3CCOCC3)cc2)n1] |
| 380 | 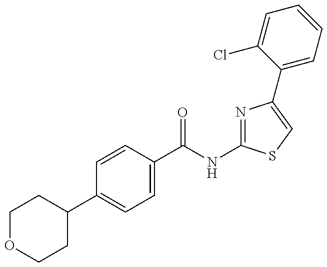 [SMILES: O=C(Nc1nc(-c2ccccc2Cl)cs1)c1ccc(C2CCOCC2)cc1] |
| 381 | 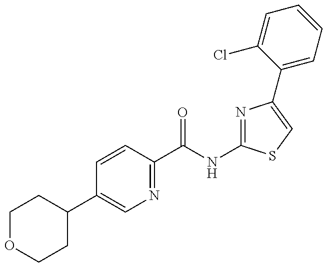 [SMILES: O=C(Nc1nc(-c2ccccc2Cl)cs1)c1ccc(C2CCOCC2)cn1] |
| 382 | 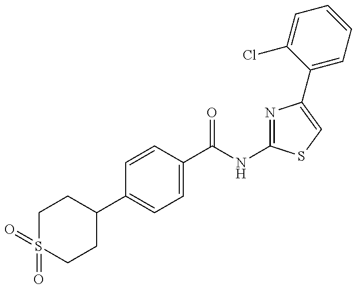 [SMILES: O=C(Nc1nc(-c2ccccc2Cl)cs1)c1ccc(C2CCS(=O)(=O)CC2)cc1] |
| 383 | 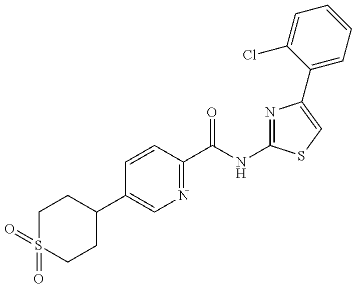 [SMILES: O=C(Nc1nc(-c2ccccc2Cl)cs1)c1ccc(C2CCS(=O)(=O)CC2)cn1] |
| 384 | 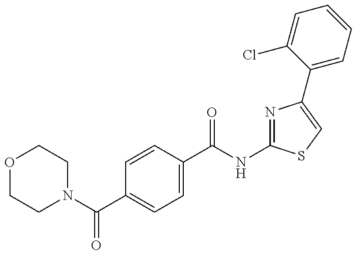 [SMILES: O=C(Nc1nc(-c2ccccc2Cl)cs1)c1ccc(C(=O)N2CCOCC2)cc1] |
| 385 | 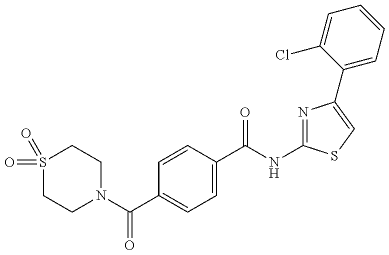 [SMILES: O=C(Nc1nc(-c2ccccc2Cl)cs1)c1ccc(C(=O)N2CCS(=O)(=O)CC2)cc1] |
| 386 | 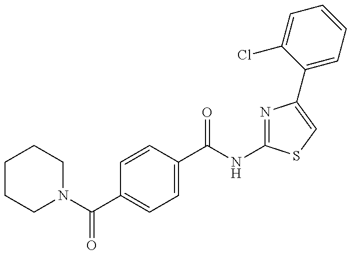 [SMILES: O=C(Nc1nc(-c2ccccc2Cl)cs1)c1ccc(C(=O)N2CCCCC2)cc1] |
| 387 | 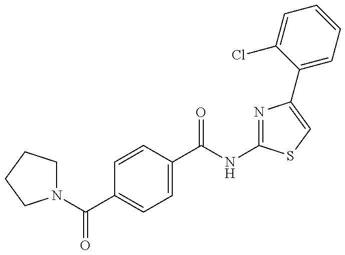 [SMILES: O=C(Nc1nc(-c2ccccc2Cl)cs1)c1ccc(C(=O)N2CCCC2)cc1] |
| 388 | 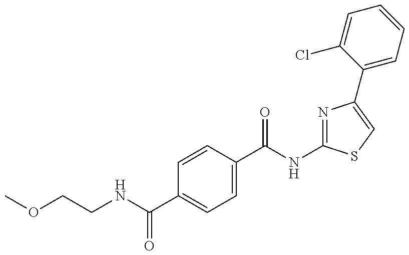 [SMILES: COCCNC(=O)c1ccc(C(=O)Nc2nc(-c3ccccc3Cl)cs2)cc1] |
| 389 | 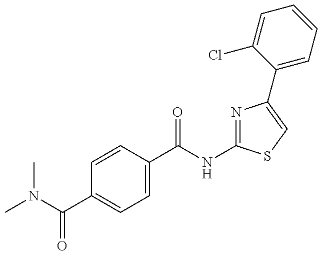 [SMILES: CN(C)C(=O)c1ccc(C(=O)Nc2nc(-c3ccccc3Cl)cs2)cc1] |
| 390 | 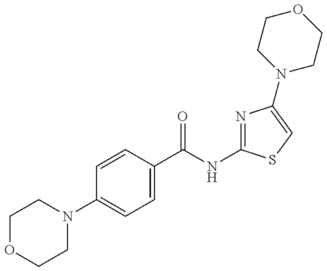 [SMILES: O=C(Nc1nc(N2CCOCC2)cs1)c1ccc(N2CCOCC2)cc1] |
| 391 | 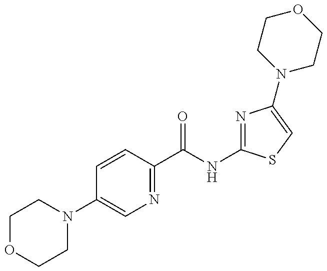 [SMILES: O=C(Nc1nc(N2CCOCC2)cs1)c1ccc(N2CCOCC2)cn1] |
| 392 | 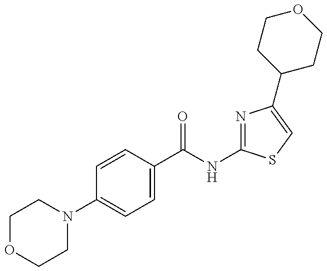 [SMILES: O=C(Nc1nc(C2CCOCC2)cs1)c1ccc(N2CCOCC2)cc1] |
| 393 | 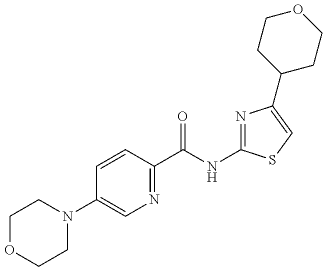 [SMILES: O=C(Nc1nc(C2CCOCC2)cs1)c1ccc(N2CCOCC2)cn1] |
| 394 | 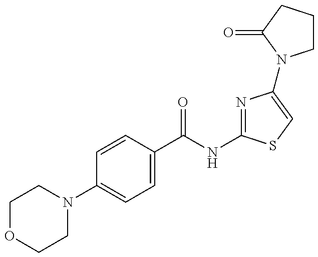 [SMILES: O=C(Nc1nc(N2CCCC2=O)cs1)c1ccc(N2CCOCC2)cc1] |
| 395 | 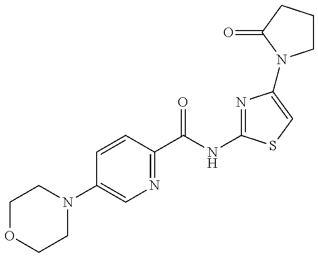 [SMILES: O=C(Nc1nc(N2CCCC2=O)cs1)c1ccc(N2CCOCC2)cn1] |
| 399 | 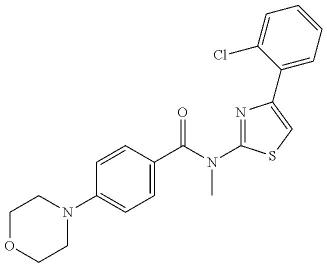 [SMILES: CN(C(=O)c1ccc(N2CCOCC2)cc1)c1nc(-c2ccccc2Cl)cs1] |
| 400 | 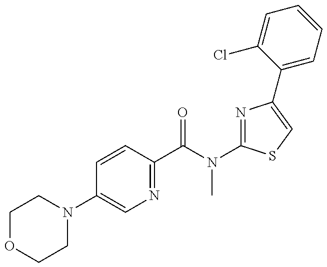 [SMILES: CN(C(=O)c1ccc(N2CCOCC2)cn1)c1nc(-c2ccccc2Cl)cs1] |
| 403 | 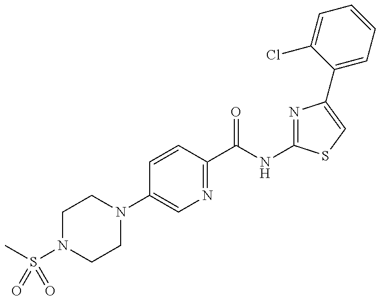 [SMILES: CS(=O)(=O)N1CCN(c2ccc(C(=O)Nc3nc(-c4ccccc4Cl)cs3)nc2)CC1] |
| 404 | 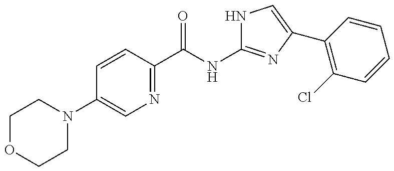 [SMILES: O=C(Nc1nc(-c2ccccc2Cl)c[nH]1)c1ccc(N2CCOCC2)cn1] |
| 405 | 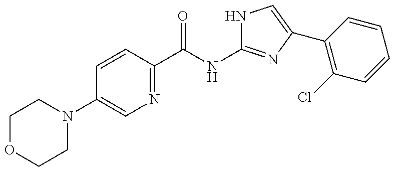 [SMILES: O=C(Nc1nc(-c2ccccc2Cl)c[nH]1)c1ccc(N2CCOCC2)cn1] |
| 408 | 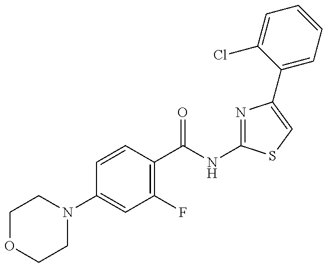 [SMILES: O=C(Nc1nc(-c2ccccc2Cl)cs1)c1ccc(N2CCOCC2)cc1F] |
| 409 | 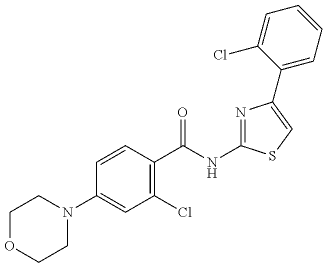 [SMILES: O=C(Nc1nc(-c2ccccc2Cl)cs1)c1ccc(N2CCOCC2)cc1Cl] |
| 410 | 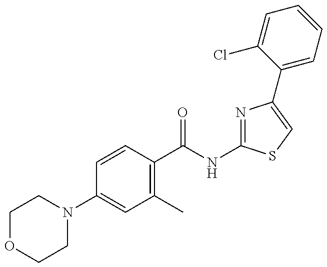 [SMILES: Cc1cc(N2CCOCC2)ccc1C(=O)Nc1nc(-c2ccccc2Cl)cs1] |
| 411 | 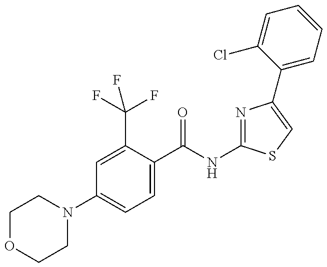 [SMILES: O=C(Nc1nc(-c2ccccc2Cl)cs1)c1ccc(N2CCOCC2)cc1C(F)(F)F] |
| 412 | 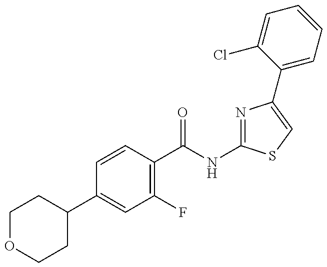 [SMILES: O=C(Nc1nc(-c2ccccc2Cl)cs1)c1ccc(C2CCOCC2)cc1F] |
| 413 | 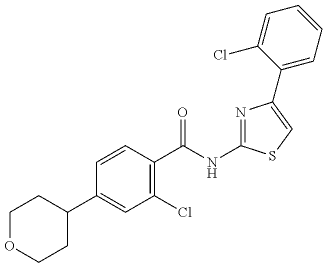 [SMILES: O=C(Nc1nc(-c2ccccc2Cl)cs1)c1ccc(C2CCOCC2)cc1Cl] |
| 414 | 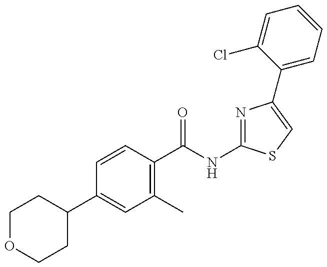 [SMILES: Cc1cc(C2CCOCC2)ccc1C(=O)Nc1nc(-c2ccccc2Cl)cs1] |
| 415 | 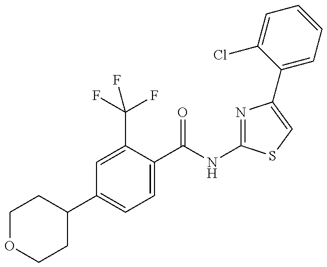 [SMILES: O=C(Nc1nc(-c2ccccc2Cl)cs1)c1ccc(C2CCOCC2)cc1C(F)(F)F] |
| 416 | 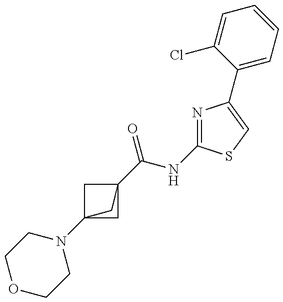 [SMILES: O=C(Nc1nc(-c2ccccc2Cl)cs1)C12CC(N3CCOCC3)(C1)C2] |
| 417 | 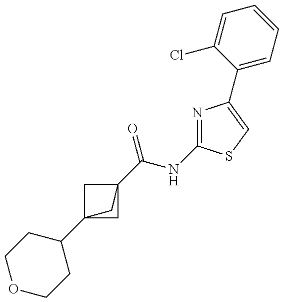 [SMILES: O=C(Nc1nc(-c2ccccc2Cl)cs1)C12CC(C3CCOCC3)(C1)C2] |
| 418 | 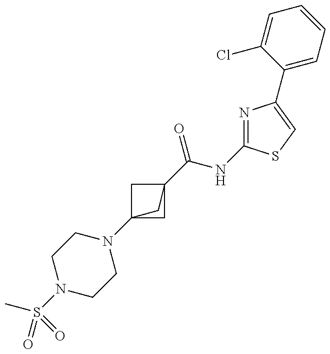 [SMILES: CS(=O)(=O)N1CCN(C23CC(C(=O)Nc4nc(-c5ccccc5Cl)cs4)(C2)C3)CC1] |
| 419 | 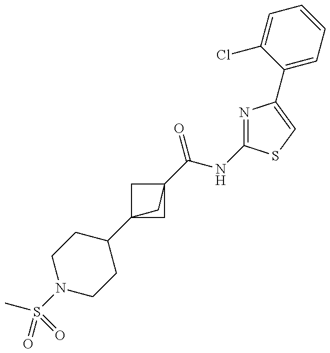 [SMILES: CS(=O)(=O)N1CCC(C23CC(C(=O)Nc4nc(-c5ccccc5Cl)cs4)(C2)C3)CC1] |
| 420 | 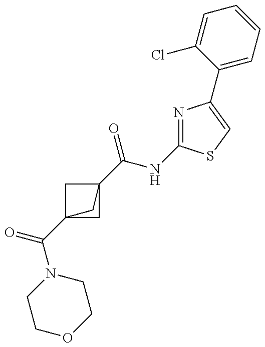 [SMILES: O=C(Nc1nc(-c2ccccc2Cl)cs1)C12CC(C(=O)N3CCOCC3)(C1)C2] |
| 421 | 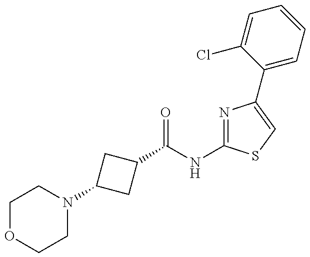 |
| 422 | 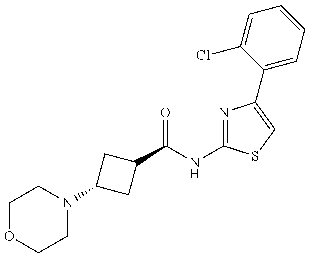 |
| 423 | 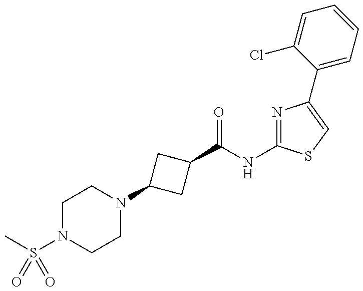 [SMILES: CS(=O)(=O)N1CCN([C@H]2C[C@@H](C(=O)Nc3nc(-c4ccccc4Cl)cs3)C2)CC1] |
| 424 | 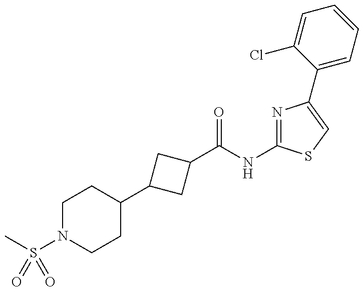 [SMILES: CS(=O)(=O)N1CCC(C2CC(C(=O)Nc3nc(-c4ccccc4Cl)cs3)C2)CC1] |
| 425 | 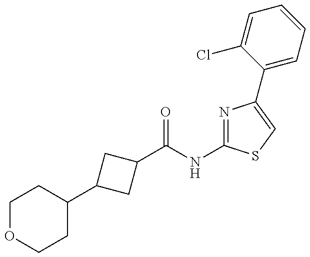 [SMILES: O=C(Nc1nc(-c2ccccc2Cl)cs1)C1CC(C2CCOCC2)C1] |
| 426 | 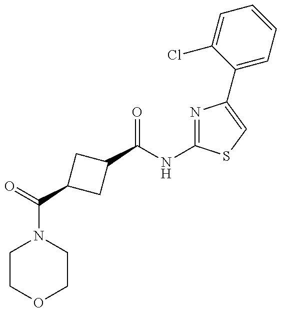 |
| 427 | 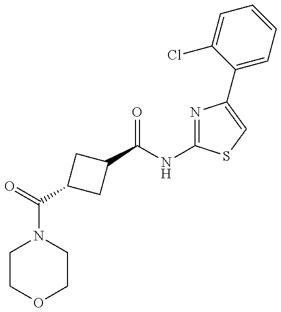 |
| 428 | 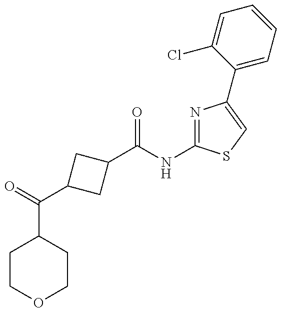 [SMILES: O=C(Nc1nc(-c2ccccc2Cl)cs1)C1CC(C(=O)C2CCOCC2)C1] |
| 429 | 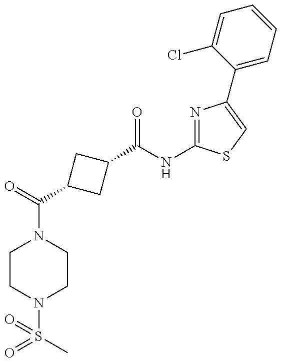 [SMILES: CS(=O)(=O)N1CCN(C(=O)[C@H]2C[C@@H](C(=O)Nc3nc(-c4ccccc4Cl)cs3)C2)CC1] |
| 430 | 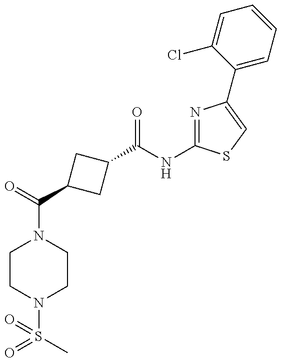 [SMILES: CS(=O)(=O)N1CCN(C(=O)[C@H]2C[C@H](C(=O)Nc3nc(-c4ccccc4Cl)cs3)C2)CC1] |
| 431 | 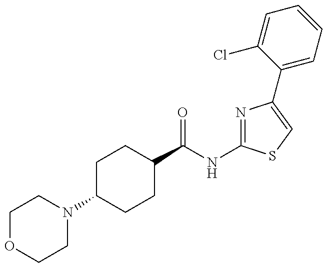 |
| 432 | 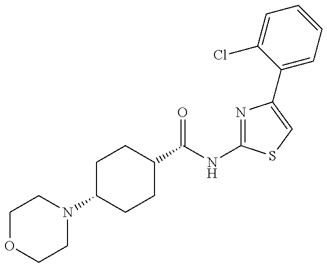 |
| 433 | 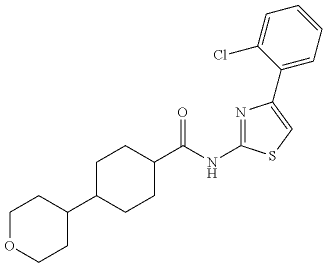 [SMILES: O=C(Nc1nc(-c2ccccc2Cl)cs1)C1CCC(C2CCOCC2)CC1] |
| 434 | 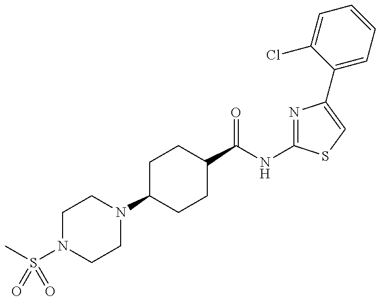 |
| 435 | 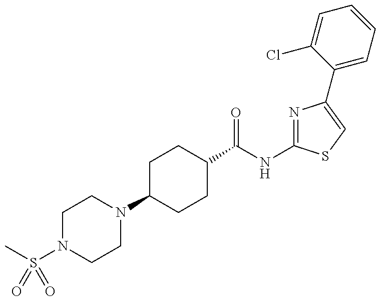 |
| 436 | 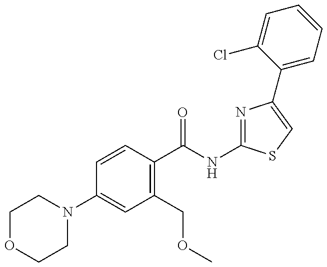 [SMILES: COCc1cc(N2CCOCC2)ccc1C(=O)Nc1nc(-c2ccccc2Cl)cs1] |
| 437 | 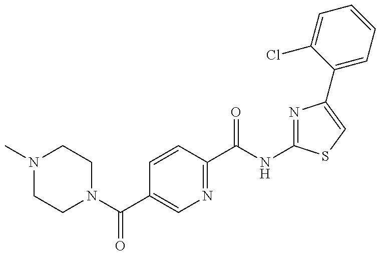 [SMILES: CN1CCN(C(=O)c2ccc(C(=O)Nc3nc(-c4ccccc4Cl)cs3)nc2)CC1] |
| 438 | 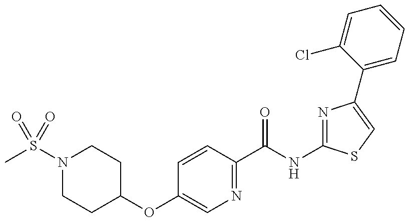 [SMILES: CS(=O)(=O)N1CCC(Oc2ccc(C(=O)Nc3nc(-c4ccccc4Cl)cs3)nc2)CC1] |
| 439 | 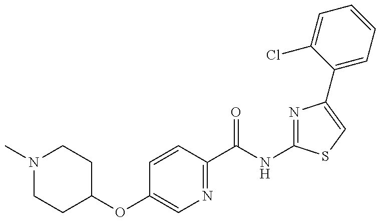 [SMILES: CN1CCC(Oc2ccc(C(=O)Nc3nc(-c4ccccc4Cl)cs3)nc2)CC1] |
| 440 | 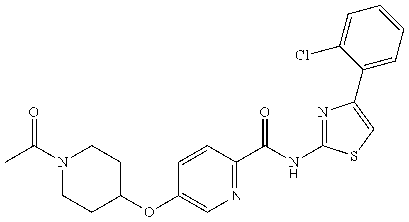 [SMILES: CC(=O)N1CCC(Oc2ccc(C(=O)Nc3nc(-c4ccccc4Cl)cs3)nc2)CC1] |
| 441 | 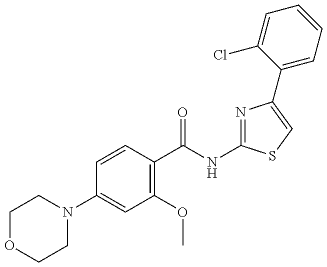 [SMILES: COc1cc(N2CCOCC2)ccc1C(=O)Nc1nc(-c2ccccc2Cl)cs1] |
| 442 | 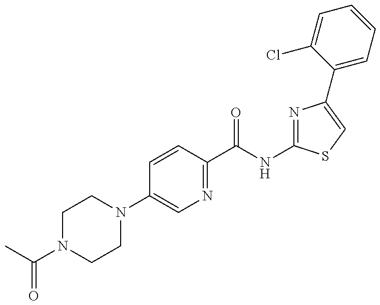 [SMILES: CC(=O)N1CCN(c2ccc(C(=O)Nc3nc(-c4ccccc4Cl)cs3)nc2)CC1] |
| 443 | 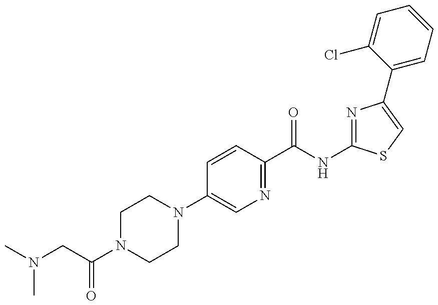 [SMILES: CN(C)CC(=O)N1CCN(c2ccc(C(=O)Nc3nc(-c4ccccc4Cl)cs3)nc2)CC1] |
| 444 | 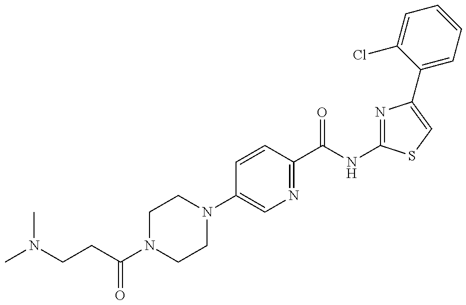 [SMILES: CN(C)CCC(=O)N1CCN(c2ccc(C(=O)Nc3nc(-c4ccccc4Cl)cs3)nc2)CC1] |
| 445 | 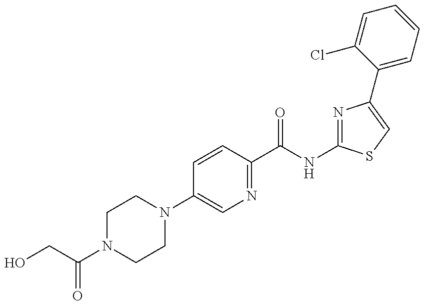 [SMILES: O=C(Nc1nc(-c2ccccc2Cl)cs1)c1ccc(N2CCN(C(=O)CO)CC2)cn1] |
| 446 | 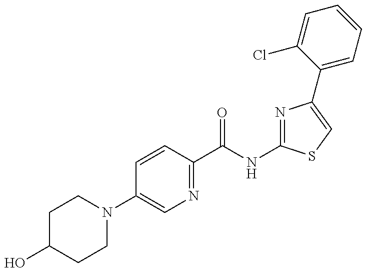 [SMILES: O=C(Nc1nc(-c2ccccc2Cl)cs1)c1ccc(N2CCC(O)CC2)cn1] |
| 447 | 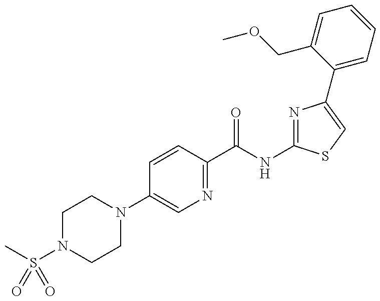 [SMILES: COCc1ccccc1-c1csc(NC(=O)c2ccc(N3CCN(S(C)(=O)=O)CC3)cn2)n1] |
| 448 | 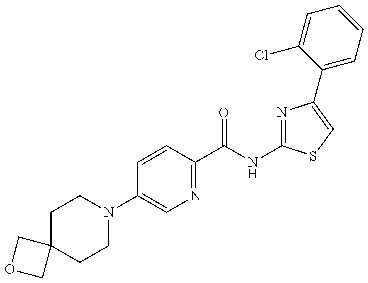 [SMILES: O=C(Nc1nc(-c2ccccc2Cl)cs1)c1ccc(N2CCC3(CC2)COC3)cn1] |
| 449 | 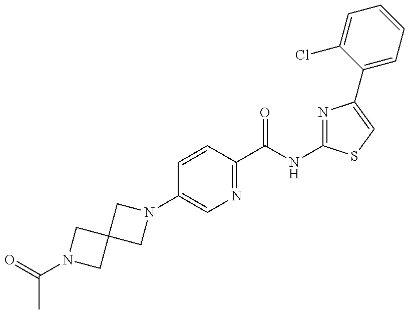 [SMILES: CC(=O)N1CC2(C1)CN(c1ccc(C(=O)Nc3nc(-c4ccccc4Cl)cs3)nc1)C2] |
| 450 | 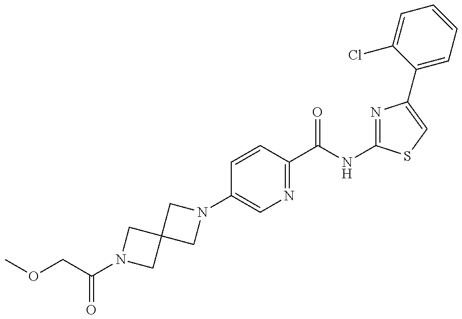 [SMILES: COCC(=O)N1CC2(C1)CN(c1ccc(C(=O)Nc3nc(-c4ccccc4Cl)cs3)nc1)C2] |
| 451 | 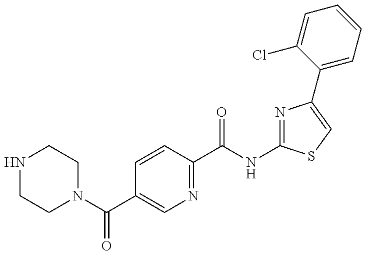 [SMILES: O=C(Nc1nc(-c2ccccc2Cl)cs1)c1ccc(C(=O)N2CCNCC2)cn1] |
| 452 | 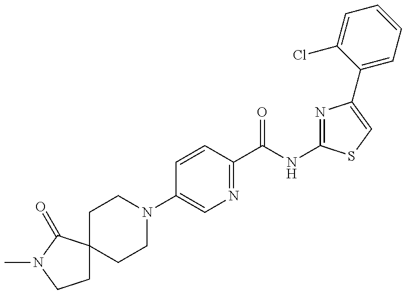 [SMILES: CN1CCC2(CCN(c3ccc(C(=O)Nc4nc(-c5ccccc5Cl)cs4)nc3)CC2)C1=O] |
| 453 | 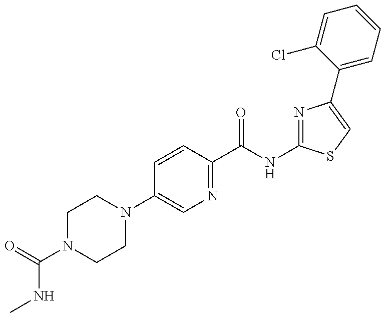 [SMILES: CNC(=O)N1CCN(c2ccc(C(=O)Nc3nc(-c4ccccc4Cl)cs3)nc2)CC1] |
| 454 | 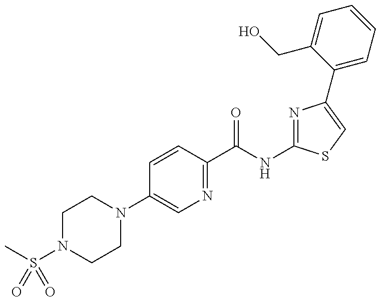 [SMILES: CS(=O)(=O)N1CCN(c2ccc(C(=O)Nc3nc(-c4ccccc4CO)cs3)nc2)CC1] |
| 455 | 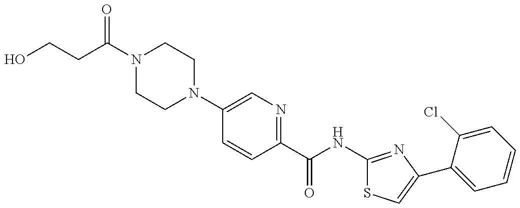 [SMILES: O=C(Nc1nc(-c2ccccc2Cl)cs1)c1ccc(N2CCN(C(=O)CCO)CC2)cn1] |
| 456 | 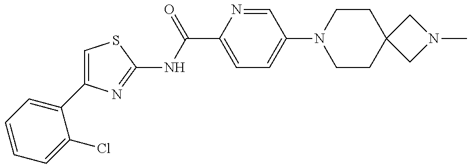 [SMILES: CN1CC2(CCN(c3ccc(C(=O)Nc4nc(-c5ccccc5Cl)cs4)nc3)CC2)C1] |
| 457 | 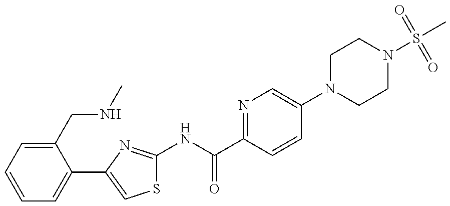 [SMILES: CNCc1ccccc1-c1csc(NC(=O)c2ccc(N3CCN(S(C)(=O)=O)CC3)cn2)n1] |
| 458 | 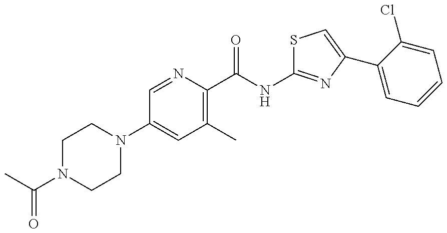 [SMILES: CC(=O)N1CCN(c2cnc(C(=O)Nc3nc(-c4ccccc4Cl)cs3)c(C)c2)CC1] |
| 459 | 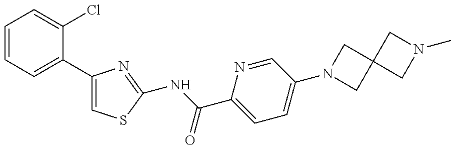 [SMILES: CN1CC2(C1)CN(c1ccc(C(=O)Nc3nc(-c4ccccc4Cl)cs3)nc1)C2] |
| 460 | 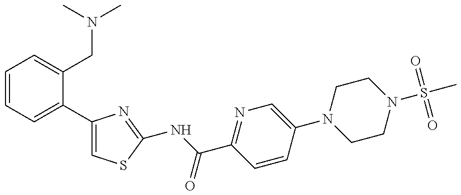 [SMILES: CN(C)Cc1ccccc1-c1csc(NC(=O)c2ccc(N3CCN(S(C)(=O)=O)CC3)cn2)n1] |
| 461 | 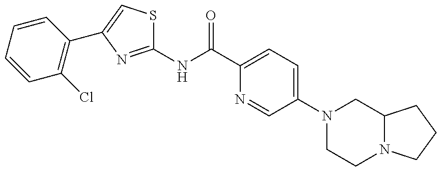 [SMILES: O=C(Nc1nc(-c2ccccc2Cl)cs1)c1ccc(N2CCN3CCCC3C2)cn1] |
| 462 | 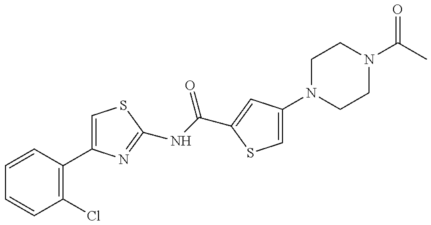 [SMILES: CC(=O)N1CCN(c2csc(C(=O)Nc3nc(-c4ccccc4Cl)cs3)c2)CC1] |
| 463 | 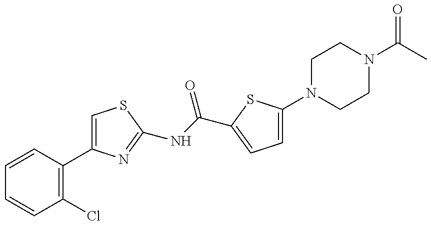 [SMILES: CC(=O)N1CCN(c2ccc(C(=O)Nc3nc(-c4ccccc4Cl)cs3)s2)CC1] |
| 464 | 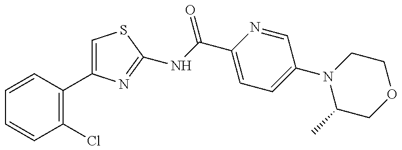 [SMILES: C[C@H]1COCCN1c1ccc(C(=O)Nc2nc(-c3ccccc3Cl)cs2)nc1] |
| 465 | 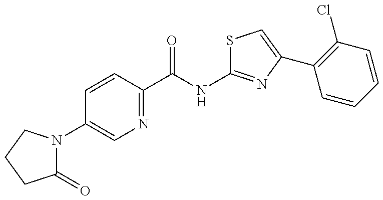 [SMILES: O=C(Nc1nc(-c2ccccc2Cl)cs1)c1ccc(N2CCCC2=O)cn1] |
| 466 | 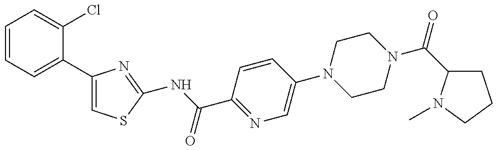 [SMILES: CN1CCCC1C(=O)N1CCN(c2ccc(C(=O)Nc3nc(-c4ccccc4Cl)cs3)nc2)CC1] |
| 467 | 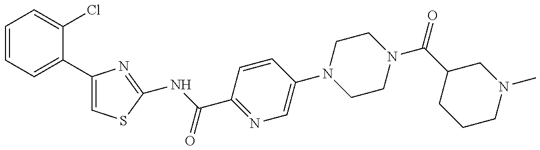 [SMILES: CN1CCCC(C(=O)N2CCN(c3ccc(C(=O)Nc4nc(-c5ccccc5Cl)cs4)nc3)CC2)C1] |
| 468 | 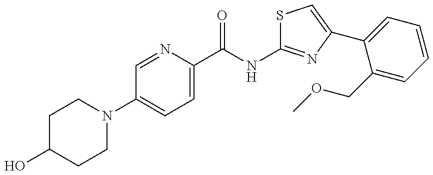 [SMILES: COCc1ccccc1-c1csc(NC(=O)c2ccc(N3CCC(O)CC3)cn2)n1] |
| 469 | 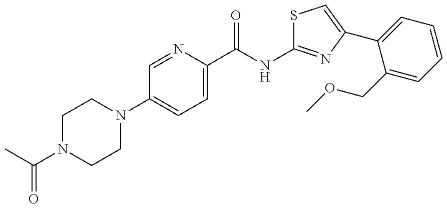 [SMILES: COCc1ccccc1-c1csc(NC(=O)c2ccc(N3CCN(C(C)=O)CC3)cn2)n1] |
| 470 | 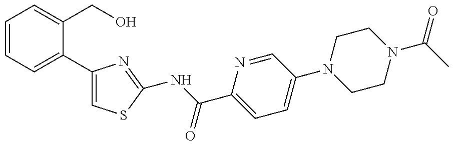 [SMILES: CC(=O)N1CCN(c2ccc(C(=O)Nc3nc(-c4ccccc4CO)cs3)nc2)CC1] |
| 471 | 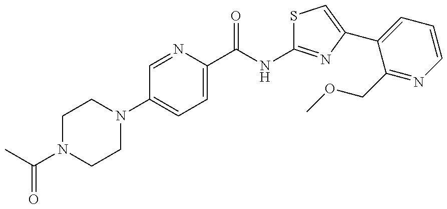 [SMILES: COCc1ncccc1-c1csc(NC(=O)c2ccc(N3CCN(C(C)=O)CC3)cn2)n1] |
| 472 | 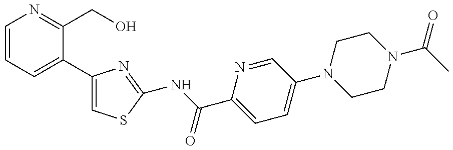 [SMILES: CC(=O)N1CCN(c2ccc(C(=O)Nc3nc(-c4cccnc4CO)cs3)nc2)CC1] |
| 473 | 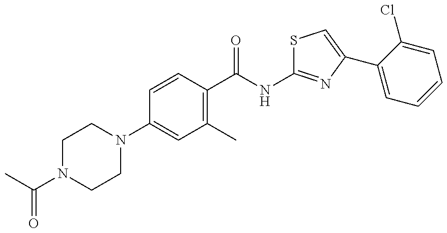 [SMILES: CC(=O)N1CCN(c2ccc(C(=O)Nc3nc(-c4ccccc4Cl)cs3)c(C)c2)CC1] |
| 474 | 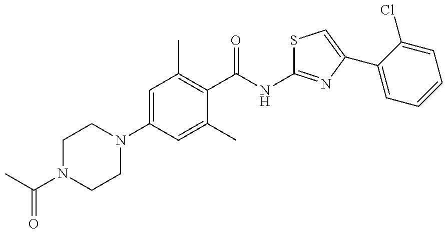 [SMILES: CC(=O)N1CCN(c2cc(C)c(C(=O)Nc3nc(-c4ccccc4Cl)cs3)c(C)c2)CC1] |
| 475 | 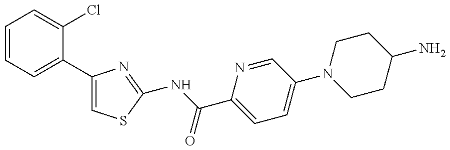 [SMILES: NC1CCN(c2ccc(C(=O)Nc3nc(-c4ccccc4Cl)cs3)nc2)CC1] |
| 476 | 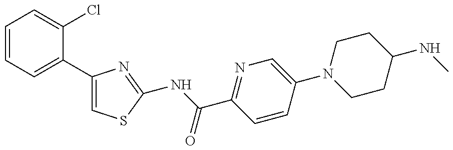 [SMILES: CNC1CCN(c2ccc(C(=O)Nc3nc(-c4ccccc4Cl)cs3)nc2)CC1] |
| 477 | 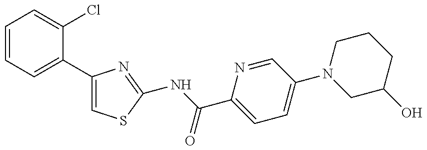 [SMILES: O=C(Nc1nc(-c2ccccc2Cl)cs1)c1ccc(N2CCCC(O)C2)cn1] |
| 478 | 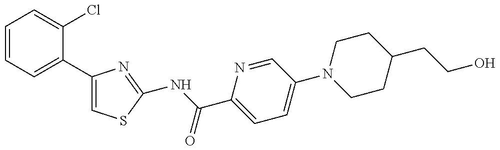 [SMILES: O=C(Nc1nc(-c2ccccc2Cl)cs1)c1ccc(N2CCC(CCO)CC2)cn1] |
| 479 | 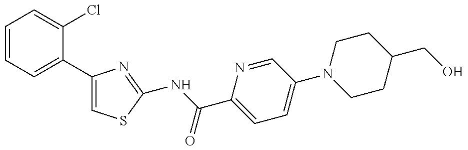 [SMILES: O=C(Nc1nc(-c2ccccc2Cl)cs1)c1ccc(N2CCC(CO)CC2)cn1] |
| 480 | 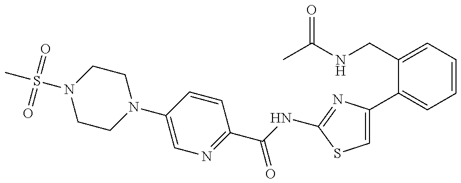 [SMILES: CC(=O)NCc1ccccc1-c1csc(NC(=O)c2ccc(N3CCN(S(C)(=O)=O)CC3)cn2)n1] |
| 481 | 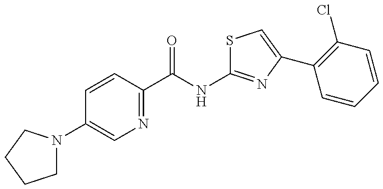 [SMILES: O=C(Nc1nc(-c2ccccc2Cl)cs1)c1ccc(N2CCCC2)cn1] |
| 482 | 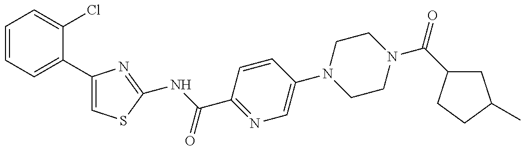 [SMILES: CC1CCC(C(=O)N2CCN(c3ccc(C(=O)Nc4nc(-c5ccccc5Cl)cs4)nc3)CC2)C1] |
| 483 | 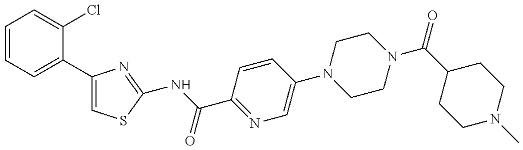 [SMILES: CN1CCC(C(=O)N2CCN(c3ccc(C(=O)Nc4nc(-c5ccccc5Cl)cs4)nc3)CC2)CC1] |
| 484 | 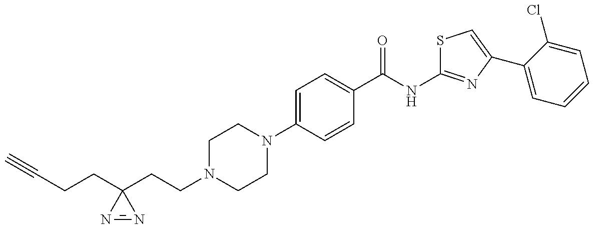 [SMILES: C#CCCC1(CCN2CCN(c3ccc(C(=O)Nc4nc(-c5ccccc5Cl)cs4)cc3)CC2)N=N1] |
| 485 | 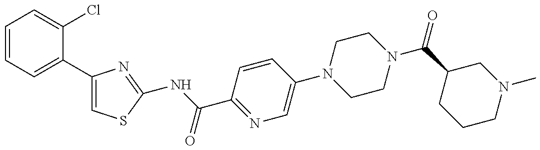 [SMILES: CN1CCC[C@@H](C(=O)N2CCN(c3ccc(C(=O)Nc4nc(-c5ccccc5Cl)cs4)nc3)CC2)C1] |
| 486 | 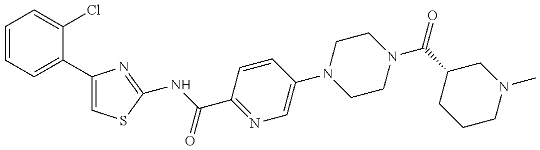 [SMILES: CN1CCC[C@H](C(=O)N2CCN(c3ccc(C(=O)Nc4nc(-c5ccccc5Cl)cs4)nc3)CC2)C1] |
| 487 | 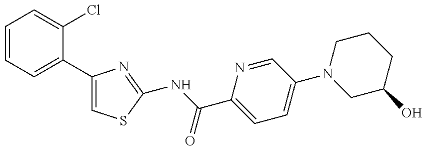 [SMILES: O=C(Nc1nc(-c2ccccc2Cl)cs1)c1ccc(N2CCC[C@@H](O)C2)cn1] |
| 488 | 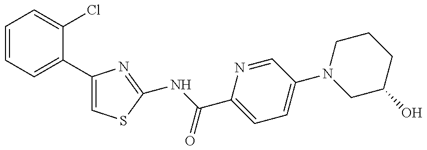 [SMILES: O=C(Nc1nc(-c2ccccc2Cl)cs1)c1ccc(N2CCC[C@H](O)C2)cn1] |

It is well understood that in structures presented in this invention wherein the carbon atom has less than 4 bonds, H atoms are present to complete the valence of the carbon. It is well understood that in structures presented in this invention wherein the nitrogen atom has less than 3 bonds, H atoms are present to complete the valence of the nitrogen.

In some embodiments, this invention is directed to the compounds listed hereinabove, pharmaceutical compositions and/or method of use thereof, wherein the compound is pharmaceutically acceptable salt, stereoisomer, tautomer, hydrate, N-oxide, prodrug, isotopic variant (deuterated analog), PROTAC, pharmaceutical product or any combination thereof. In some embodiments, the compounds are Collagen I translation inhibitors. In some embodiments, the compounds are Collagen I, II, II, IV, or V translation inhibitors; each represents a separate embodiment according to this invention. In some embodiments, the compounds are selective to Collagen I, II, II, IV, or V; each represents a separate embodiment according to this invention. In some embodiments, the compounds are selective to Collagen I. In some embodiments, the compounds are selective to Collagen IA. In some embodiments, the compounds are selective to Collagen IA1.

In various embodiments, the A ring of formula I, II, and/or VII is phenyl, naphthyl, pyridinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, isoquinoline, pyrazolyl, pyrrolyl, furanyl, thiophene-yl, isoquinolinyl, indolyl, 111-indole, isoindolyl, naphthyl, anthracenyl, benzimidazolyl, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, cinnolinyl, phthalazinyl, quinolinyl, isoquinolinyl, 2,3-dihydroindenyl, indenyl, tetrahydronaphthyl, 3,4-dihydro-2H-benzo[b][1,4]dioxepine, benzo[d][1,3]dioxole, acridinyl, benzofuranyl, 1-benzofuran, isobenzofuranyl, benzofuran-2(3H)-one, benzothiophenyl, benzoxadiazole, benzo[c][1,2,5]oxadiazolyl, benzo[c]thiophenyl, benzodioxolyl, benzo[d][1,3]dioxole, thiadiazolyl, [1,3]oxazolo[4,5-b]pyridine, oxadiaziolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazopyridin, imidazo[1,2-a]pyridine, 1H-imidazo[4,5-b]pyridine, 1H-imidazo[4,5-c]pyridine, 3H-imidazo[4,5-c]pyridine, pyrazolopyridine, pyrazolo[1,5-a]pyridine, imidazo[1,2-a]pyrazine, imidazo[1,2-a]pyrimidine, 1H-pyrrolo[2,3-b]pyridine, pyrido[2,3-b]pyrazine, pyrido[2,3-b]pyrazin-3(4H)-one, 4H-thieno[3,2-b]pyrrole, quinoxalin-2(1H)-one, pyrrolopyridine, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine; each represents a separate embodiment according to this invention; or A is C₃-C₈ cycloalkyl (e.g. cyclohexyl, cyclopentyl, bicyclo[1.1.1]pentyl, cyclobutyl) or C₃-C₈ heterocyclic ring including but not limited to: tetrahydropyran, piperidine, 1-methylpiperidine, tetrahydrothiophene 1,1-dioxide, pyrrolidin-2-one, piperazine, 1-(piperidin-1-yl)ethanone or morpholine; each represents a separate embodiment according to this invention. In some embodiments, A is a phenyl. In some embodiments, A is a C₃-C₈ heterocyclic ring. In some embodiments, A is tetrahydro-2H-pyran. In other embodiments, A is azetidine. In other embodiments, A is piperidine.

In various embodiments, the B ring of formula I is phenyl, naphthyl, pyridinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, isoquinoline, pyrazolyl, pyrrolyl, furanyl, thiophene-yl, isoquinolinyl, indolyl, 1H-indole, isoindolyl, naphthyl, anthracenyl, benzimidazolyl, 2,3-dihydro-1H-benzo[d]imidazolyl, tetrahydronaphthyl 3,4-dihydro-2H-benzo[b][1,4]dioxepine, benzofuran-2(3H)-one, benzo[d][1,3]dioxole, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, cinnolinyl, phthalazinyl, quinolinyl, isoquinolinyl, acridinyl, benzofuranyl, 1-benzofuran, isobenzofuranyl, benzothiophenyl, benzoxadiazole, benzo[c][1,2,5]oxadiazolyl, benzo[c]thiophenyl, benzodioxolyl, thiadiazolyl, [1,3]oxazolo[4,5-b]pyridine, oxadiaziolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazo[1,2-a]pyridine, 1H-imidazo[4,5-b]pyridine, 3H-imidazo[4,5-b]pyridine, 3H-imidazo[4,5-c]pyridine, pyrazolo[1,5-a]pyridine, imidazo[1,2-a]pyrazine, imidazo[1,2-a]pyrimidine, pyrido[2,3-b]pyrazin or pyrido[2,3-b]pyrazin-3(4H)-one, 4H-thieno[3,2-b]pyrrole, quinoxalin-2(1H)-one, 1,2,3,4-tetrahydroquinoxaline, 1-(pyridin-1(2H)-yl)ethanone,1H-pyrrolo[2,3-b]pyridine, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine, C₃-C₈ cycloalkyl, or C₃-C₈ heterocyclic ring including but not limited to: tetrahydropyran, piperidine, 1-methylpiperidine, tetrahydrothiophene 1,1-dioxide, 1-(piperidin-1-yl)ethanone, bicyclo[1.1.1]pentyl, cyclobutyl, cyclohexyl or morpholine; each represents a separate embodiment according to this invention. In some embodiments, B is a C₃-C₈ heterocyclic ring. In some embodiments, B is piperidine. In some embodiments, B is piperazine. In some embodiments, B is pyrrolidin-2-one. In some embodiments, B is tetrahydro-2H-pyran. In some embodiments, B is azetidine. In some embodiments, B is pyrimidine. In some embodiments, B is a phenyl. In some embodiments, B is a pyridinyl. In some embodiments, B is a 2-pyridinyl. In some embodiments, B is a thiophenyl.

In various embodiments, compound of formula I-VIII is substituted by R₁. In various embodiments, compound of formula I-III, VI and/or VII is substituted by R₂. In various embodiments, compound of formula I-V is substituted by R₃. In various embodiments, compound of formula I-III, and/or VI-VII is substituted by R₄. Single substituents can be present at the ortho, meta, or para positions.

In various embodiments, R₁ of formula I-VII and/or R₂ of formula I-III and/or VI-VII are each independently H.

In various embodiments, R₁ of formula I-VIII and/or R₂ of formula I-III, VI and/or VII are each independently H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃- C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl, CHF₂, C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl), substituted or unsubstituted benzyl; each possibility represents a separate embodiment according to this invention. In some embodiment, R₁ and/or R₂ may be further substituted by at least one selected from: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl and (benzyloxy)phenyl; each possibility represents a separate embodiment according to this invention.

In some embodiments, R₁ and R₂ are joined together to form a 5 or 6 membered substituted or unsubstituted, aliphatic or aromatic, carbocyclic or heterocyclic ring. In some embodiments, R₁ and R₂ are joined together to form a 5 or 6 membered heterocyclic ring. In some embodiments, R₁ and R₂ are joined together to form a pyrrol ring. In some embodiments, R₁ and R₂ are joined together to form a [1,3]dioxole ring. In some embodiments, R₁ and R₂ are joined together to form a 1,4-dioxane ring. In some embodiments, R₁ and R₂ are joined together to form a 2,3-dihydro-1,4-dioxine ring. In some embodiments, R₁ and R₂ are joined together to form a furan-2(3H)-one ring. In some embodiments, R₁ and R₂ are joined together to form a benzene ring. In some embodiments, R₁ and R₂ are joined together to form a pyridine ring. In some embodiments, R₁ and R₂ are joined together to form a morpholine ring. In some embodiments, R₁ and R₂ are joined together to form a piperazine ring. In some embodiments, R₁ and R₂ are joined together to form an imidazole ring. In some embodiments, R₁ and R₂ are joined together to form a pyrrole ring. In some embodiments, R₁ and R₂ are joined together to form a cyclohexene ring. In some embodiments, R₁ and R₂ are joined together to form a pyrazine ring.

In various embodiments, R₃ of formula I-V; and/or R₄ of formula I-III VII-IX; are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)NH(CH₃)₂₀—CH₃, C(O)N(R₁₀)(R₁₁), C(O)-piperidine, C(O)-pyrrolidine, C(O)N(CH₃)₂, C(O)-piperazine, SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl, CHF₂, C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1- or 4-(methylsulfonyl)piperidine, 2-methoxy-1-(piperazin-1-yl)ethenone, 1-, or 4-methylpiperazine, 1- or 4-(methylsulfonyl)piperazine, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, N-methylpiperazine-1-carboxamide, piperidin-4-ol, morpholine, 3-methylmorpholine, 3-hydroxypiperidine, tetrahydro-2H-thiopyran 1,1-dioxide, tetrahydro-2H-pyrane, pyrazole, thiazole, imidazole, pyrrolidine, pyrrolidinone, octahydropyrrolo[1,2-a]pyrazine, 6-methyl-2,6-diazaspiro[3.3]heptane, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl), or substituted or unsubstituted benzyl; each possibility represents a separate embodiment of this invention. In some embodiment, R₃ and/or R₄ may be further substituted by at least one selected from: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl and (benzyloxy)phenyl; each possibility represents a separate embodiment of this invention.

In some embodiments, R₃ and R₄ are joined together to form a 5 or 6 membered substituted or unsubstituted, aliphatic or aromatic, carbocyclic or heterocyclic ring. In some embodiments, R₃ and R₄ are joined together to form a 5 or 6 membered carbocyclic ring. In some embodiments, R₃ and R₄ are joined together to form a 5 or 6 membered heterocyclic ring. In some embodiments, R₃ and R₄ are joined together to form a dioxole ring. [1,3]dioxole ring. In some embodiments, R₃ and R₄ are joined together to form a dihydrofuran-2(3H)-one ring. In some embodiments, R₃ and R₄ are joined together to form a furan-2(3H)-one ring. In some embodiments, R₃ and R₄ are joined together to form a benzene ring. In some embodiments, R₃ and R₄ are joined together to form an imidazole ring. In some embodiments, R₃ and R₄ are joined together to form a pyridine ring. In some embodiments, R₃ and R₄ are joined together to form a thiophene ring. In some embodiments, R₃ and R₄ are joined together to form a furane ring. In some embodiments, R₃ and R₄ are joined together to form a pyrrole ring. In some embodiments, R₃ and R₄ are joined together to form a pyrazole ring. In some embodiments, R₃ and R₄ are joined together to form a cyclohexene ring. In some embodiments, R₃ and R₄ are joined together to form a cyclopentene ring. In some embodiments, R₄ and R₃ are joined together to form a dioxepine ring.

In some embodiments, R₅ of compound of formula I is H, R₂₀, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl; each represents a separate embodiment according to this invention. In some embodiments, R₅ may be further substituted by at least one selected from: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl and (benzyloxy)phenyl; each possibility represents a separate embodiment of this invention.

In various embodiments, n of compound of formula I-IV and/or VI-VII is 1. In some embodiments, n is 0 or 1. In some embodiments, n is between 1 and 3. In some embodiments, n is between 1 and 4. In some embodiments, n is between 1 and 2. In some embodiments, n is between 0 and 3. In some embodiments, n is between 0 and 4. In some embodiments, n is 1. In some embodiments, n is 2. In some embodiments, n is 3. In some embodiments, n is 4.

In various embodiments, m of compound of formula I-III and/or VI-VII is 0. In some embodiments, m is 0 or 1. In some embodiments, m is between 1 and 3. In some embodiments, m is between 1 and 4. In some embodiments, m is between 0 and 2. In some embodiments, m is between 0 and 3. In some embodiments, m is between 0 and 4. In some embodiments, m is 1. In some embodiments, m is 2. In some embodiments, m is 3. In some embodiments, m is 4.

In various embodiments, 1 of compound of formula I-IV is 0. In some embodiments, 1 is 0 or 1. In some embodiments, 1 is between 1 and 3. In some embodiments, 1 is between 1 and 4. In some embodiments, 1 is 1 or 2. In some embodiments, 1 is between 0 and 3. In some embodiments, 1 is between 0 and 4. In some embodiments, 1 is 1. In some embodiments, 1 is 2. In some embodiments, 1 is 3. In some embodiments, 1 is 4.

In various embodiments, k of compound of formula I-III and/or VI-VII is 0. In some embodiments, k is 0 or 1. In some embodiments, k is between 1 and 3. In some embodiments, k is between 1 and 4. In some embodiments, k is between 0 and 2. In some embodiments, k is between 0 and 3. In some embodiments, k is between 0 and 4. In some embodiments, k is 1. In some embodiments, k is 2. In some embodiments, k is 3. In some embodiments, k is 4.

It is understood that for heterocyclic rings, n, m, 1 and/or k are limited to the number of available positions for substitution, i.e. to the number of CH or NH groups minus one. Accordingly, if A and/or B rings are, for example, furanyl, thiophenyl or pyrrolyl, n, m, 1 and k are between 0 and 2; and if A and/or B rings are, for example, oxazolyl, imidazolyl or thiazolyl, n, m, 1 and k are either 0 or 1; and if A and/or B rings are, for example, oxadiazolyl or thiadiazolyl, n, m, 1 and k are 0.

In various embodiments, R₈ of compound of formula I-VIII is CH₂. In some embodiments, R₈ is CH₂CH₂. In some embodiments, R₈ is CH₂CH₂CH₂. In some embodiments, R₈ is CH₂CH₂CH₂CH₂.

In various embodiments, p of compound of formula I-VII is 1. In some embodiments, p is 2. In some embodiments, p is 3. In some embodiments, p is 4. In some embodiments, p is 5. In some embodiments, p is between 1 and 3. In some embodiments, p is between 1 and 5. In some embodiments, p is between 1 and 10.

In some embodiments, R₉ of compound of formula I-VII is C≡C. In some embodiments, R₉ is C≡C—C≡C. In some embodiments, R₉ is CH═CH. In some embodiments, R₉ is CH═CH—CH═CH.

In some embodiments, q of compound of formula I-VIII is 2. In some embodiments, q is 4. In some embodiments, q is 6. In some embodiments, q is 8. In some embodiments, q is between 2 and 6.

In various embodiments, R₁₀ of compound of formula I-VII is H. In some embodiments, R₁₀ is OH. In some embodiments, R₁₀ is substituted or unsubstituted C₁-C₅ linear or branched alkyl. In some embodiments, R₁₀ is methyl. In some embodiments, R₁₀ is ethyl. In some embodiments, R₁₀ is propyl. In some embodiments, R₁₀ is isopropyl. In some embodiments, R₁₀ is butyl. In some embodiments, R₁₀ is isobutyl. In some embodiments, R₁₀ is t-butyl. In some embodiments, R₁₀ is cyclopropyl. In some embodiments, R₁₀ is pentyl. In some embodiments, R₁₀ is isopentyl. In some embodiments, R₁₀ is neopentyl. In some embodiments, R₁₀ is benzyl. In some embodiments, R₁₀ is CH₂—CH₂—O—CH₃. In some embodiments, R₁₀ is substituted or unsubstituted C₃-C₈ heterocyclic ring. In some embodiments, R₁₀ is 1-(methylsulfonyl)piperidine. In some embodiments, R₁₀ is 1-(methylsulfonyl)piperazine. In some embodiments, R₁₀ is tetrahydro-2H-pyrane. In some embodiments, R₁₀ is morpholine. In some embodiments, R₁₀ is thiomorpholine 1,1-dioxide. In some embodiments, R₁₀ is methyl-pyrrolidine. In some embodiments, R₁₀ is methyl-piperidine. In some embodiments, R₁₀ is C(O)-alkyl. In some embodiments, R₁₀ is S(O)₂-alkyl. In some embodiments, R₁₀ may be further substituted by at least one selected from: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl and (benzyloxy)phenyl; each possibility represents a separate embodiment of this invention.

In various embodiments, R₁₁ of compound of formula I-VIII is H. In some embodiments, R₁ is OH. In some embodiments, R₁ is C₁-C₅ linear or branched alkyl. In some embodiments, R₁ is methyl. In some embodiments, R₁ is ethyl. In some embodiments, R₁₀ is propyl. In some embodiments, R₁ is isopropyl. In some embodiments, R₁₁ is butyl. In some embodiments, R₁₁ is isobutyl. In some embodiments, R₁₁ is t-butyl. In some embodiments, R₁₁ is cyclopropyl. In some embodiments, R₁ is pentyl. In some embodiments, R₁₁ is isopentyl. In some embodiments, R₁ is neopentyl. In some embodiments, R₁ is benzyl. In some embodiments, R₁ is CH₂—CH₂—O—CH₃. In some embodiments, R₁₁ is substituted or unsubstituted C₃-C₈ heterocyclic ring. In some embodiments, R₁₁ is 1-(methylsulfonyl)piperidine. In some embodiments, R₁ is 1-(methylsulfonyl)piperazine. In some embodiments, R₁ is tetrahydro-2H-pyrane. In some embodiments, R₁₁ is morpholine. In some embodiments, R₁ is thiomorpholine 1,1-dioxide. In some embodiments, R₁₁ is methyl-pyrrolidine. In some embodiments, R₁ is methyl-piperidine. In some embodiments, R₁₁ is C(O)-alkyl. In some embodiments, R₁ is S(O)₂-alkyl. In some embodiments, R₁₁ may be further substituted by at least one selected from: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl and (benzyloxy)phenyl; each possibility represents a separate embodiment of this invention.

In some embodiments, R₁₀ and R₁₁ of formula I-VII are joined to form a substituted or unsubstituted C₃-C₈ heterocyclic ring. In other embodiments, R₁₀ and R₁₁ are joined to form a morpholine ring. In other embodiments, R₁₀ and R₁₁ are joined to form a piperazine ring. In other embodiments, R₁₀ and R₁₁ are joined to form a substituted piperazine ring. In other embodiments, R₁₀ and R₁₁ are joined to form a piperidine ring. In other embodiments, R₁₀ and R₁₁ are joined to form an unsubstituted pyrrolidine ring. In other embodiments, R₁₀ and R₁₁ are joined to form a 1-methylpyrrolidin-2-one ring. In other embodiments, R₁₀ and R₁₁ are joined to form an oxetane. In other embodiments, R₁₀ and R₁₁ are joined to form an azetidine. In other embodiments, R₁₀ and R₁₁ are joined to form a 1-methylazetidine. In some embodiments, R₁₀ and/or R₁₁ may be further substituted by at least one selected from: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl and (benzyloxy)phenyl; each possibility represents a separate embodiment of this invention.

In some embodiments, R of formula I-VIII is H. In other embodiments, R is OH. In other embodiments, R is F. In other embodiments, R is Cl. In other embodiments, R is Br. In other embodiments, R is I. In other embodiments, R is CN. In other embodiments, R is CF₃. In other embodiments, R is NO₂. In other embodiments, R is NH₂. In other embodiments, R is NH(R₁₀). In other embodiments, R is NH(CH₃). In other embodiments, R is N(R₁₀)(R₁₁). In other embodiments, R is R₂₀. In other embodiments, R is C₁-C₅ linear or branched, substituted or unsubstituted alkyl. In other embodiments, R is methyl. In other embodiments, R is ethyl. In other embodiments, R is substituted alkyl. In other embodiments, R is CH₂CH₂OH. In other embodiments, R is CH₂CH₂OCH₃. In other embodiments, R is R₈—R₁₀. In other embodiments, R is CH₂—OH. In other embodiments, R is CH₂CH₂—OH. In other embodiments, R is C(O)—R₁₀. In other embodiments, R is C(O)-methylpyrroldine. In other embodiments, R is C(O)-methylpiperidine. In other embodiments, R is C(O)—CH₃. In other embodiments, R is —R₈—O—R₁₀. In other embodiments, R is CH₂—CH₂—O—CH₃. In other embodiments, R is C₁-C₅ substituted or unsubstituted C(O)-alkyl. In other embodiments, R is C(O)—CH₂CH₂—OCH₃. In other embodiments, R is C(O)—CH₃. In other embodiments, R is C(O)—CH₂—N(CH₃)₂. In other embodiments, R is C(O)—CH₂—CH₂—N(CH₃)₂. In other embodiments, R is C(O)—CH₂—OH. In other embodiments, R is C(O)—R₈—R₁₀. In other embodiments, R is C(O)—CH₂CH₂—OH. In other embodiments, R is C(O)-substituted or unsubstituted C₃-C₈ heterocyclic ring. In other embodiments, R is C(O)-methylpyrroldine. In other embodiments, R is C(O)-methylpiperidine. In other embodiments, R is SO₂-alkyl. In other embodiments, R is SO₂—CH₃. In other embodiments, R is C₁-C₅ substituted or unsubstituted C(O)—NH-alkyl. In other embodiments, R is C(O)—NH—CH₃. In other embodiments, R is C₁-C₅ linear or branched C(O)—O-alkyl. In other embodiments, R is C(O)—O-tBu. In other embodiments, R is C₁-C₅ linear or branched alkoxy. In other embodiments, R is —R₈—O—R₁₀. In other embodiments, R is CH₂—CH₂—O—CH₃. In other embodiments, R is C₁-C₅ linear or branched haloalkyl. In other embodiments, R is CF₃. In other embodiments, R is CF₂CH₃. In other embodiments, R is CH₂CF₃. In other embodiments, R is CF₂CH₂CH₃. In other embodiments, R is CH₂CH₂CF₃. In other embodiments, R is CF₂CH(CH₃)₂. In other embodiments, R is CF(CH₃)—CH(CH₃)₂. In other embodiments, R is R₈-aryl. In other embodiments, R is CH₂-Ph. In other embodiments, R is substituted or unsubstituted aryl. In other embodiments, R is phenyl. In other embodiments, R is substituted or unsubstituted heteroaryl. In other embodiments, R is pyridine. In other embodiments, R is 2, 3, or 4-pyridine. In some embodiments, R may be further substituted by at least one selected from: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl and (benzyloxy)phenyl; each possibility represents a separate embodiment of this invention. In some embodiment, two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring; each represents a separate embodiment according to this invention.

In some embodiments, X₁ of compound of formula III-VIII is N. In other embodiments, X₁ is C.

In some embodiments, X₂ of compound of formula III-VIII is N. In other embodiments, X₂ is C.

In some embodiments, X₃ of compound of formula II-VIII is N. In other embodiments, X₃ is C.

In some embodiments, X₄ of compound of formula II-VIII is C. In other embodiments, X₄ is N.

In some embodiments, X₅ of compound of formula II-VIII is C. In other embodiments, X₅ is N.

It is understood that H atoms are added where necessary, in order to complete the valence of the unsubstituted carbon atoms of X₁-X₅ of any one of formulas II-VIII.

In some embodiments, X₆ of compound of formula VI-VIII is O. In other embodiments, X₆ is CH₂. In other embodiments, X₆ is CHR. In other embodiments, X₆ is CH(OH). In other embodiments, X₆ is CH(NH₂). In other embodiments, X₆ is CH(NH(CH₃)). In other embodiments, X₆ is C(R₁₀)(R₁₁). In other embodiments, X₆ is C(H)CH₂CH₂—OH. In other embodiments, X₆ is C(H)CH₂—OH. In other embodiments, X₆ is 1-methylpyrrolidin-2-one. In other embodiments, X₆ is oxetane. In other embodiments, X₆ is NH. In other embodiments, X₆ is N—R. In other embodiments, X₆ is N—CH₃. In other embodiments, X₆ is N—SO₂—CH₃. In other embodiments, X₆ is N—R₂₀. In other embodiments, X₆ is N—C(O)O-tBu. In other embodiments, X₆ is N—C(O)—CH₂CH₂—OCH₃. In other embodiments, X₆ is N—CH₂CH₂—OCH₃. In other embodiments, X₆ is N—C(O)—CH₃. In other embodiments, X₆ is C₁-C₅ substituted or unsubstituted N—C(O)—NH-alkyl. In other embodiments, X₆ is N—C(O)—NH—CH₃. In other embodiments, X₆ is N—C(O)—CH₂—N(CH₃)₂. In other embodiments, X₆ is N—C(O)—CH₂—CH₂—N(CH₃)₂. In other embodiments, X₆ is N—C(O)—CH₂CH₂—OH. In other embodiments, X₆ is N—C(O)—CH₂—OH. In other embodiments, X₆ is N—C(O)—R₁₀. In other embodiments, X₆ is 1-methylazetidine. In other embodiments, X₆ is N—C(O)-1-methyl-2-pyrrolidine. In other embodiments, X₆ is N—C(O)-1-methyl-3-pyrrolidine. In other embodiments, X₆ is N—C(O)-1-methyl-3-piperidine. In other embodiments, X₆ is N—C(O)-1-methyl-4-piperidine. In other embodiments, X₆ is N—R₂₀.

In some embodiments, at least one of X₁-X₂ is N.

In some embodiments, at least one of X₃-X₅ is N. In some embodiments, at least two of X₃-X₅ are N.

In some embodiments, Qi of formula I is S. In other embodiments, Qi is 0. In other embodiments, Qi is NH.

In some embodiments, G=X of formula I is C═O. In other embodiments, G=X is C═S. In other embodiments, G=X is S═O. In other embodiments, G=X is SO₂.

As used herein, “single or fused aromatic or heteroaromatic ring systems” can be any such ring, including but not limited to phenyl, naphthyl, pyridinyl, (2-, 3-, and 4-pyridinyl), quinolinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, pyrazolyl, pyrrolyl, furanyl, thiophene-yl, quinolinyl, isoquinolinyl, 2,3-dihydroindenyl, indenyl, tetrahydronaphthyl, 3,4-dihydro-2H-benzo[b][1,4]dioxepine benzodioxolyl, benzo[d][1,3]dioxole, tetrahydronaphthyl, indolyl, 1H-indole, isoindolyl, anthracenyl, benzimidazolyl, 2,3-dihydro-1H-benzo[d]imidazolyl, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, 1,2,3,4-tetrahydroquinoxaline, 1-(pyridin-1(2H)-yl)ethanone, cinnolinyl, phthalazinyl, quinolinyl, isoquinolinyl, acridinyl, benzofuranyl, 1-benzofuran, isobenzofuranyl, benzofuran-2(3H)-one, benzothiophenyl, benzoxadiazole, benzo[c][1,2,5]oxadiazolyl, benzo[c]thiophenyl, benzodioxolyl, thiadiazolyl, [1,3]oxazolo[4,5-b]pyridine, oxadiaziolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazo[1,2-a]pyridine, 1H-imidazo[4,5-b]pyridine, 1H-imidazo[4,5-c]pyridine, 3H-imidazo[4,5-c]pyridine, pyrazolo[1,5-a]pyridine, imidazo[1,2-a]pyrazine, imidazo[1,2-a]pyrimidine, 1H-pyrrolo[2,3-b]pyridine, pyrido[2,3-b]pyrazine, pyrido[2,3-b]pyrazin-3(4H)-one, 4H-thieno[3,2-b]pyrrole, quinoxalin-2(1H)-one, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine, 3-methyl-4H-1,2,4-triazole, 5-methyl-1,2,4-oxadiazole, etc.

As used herein, the term “alkyl” can be any straight- or branched-chain alkyl group containing up to about 30 carbons unless otherwise specified. In various embodiments, an alkyl includes C₁-C₅ carbons. In some embodiments, an alkyl includes C₁-C₆ carbons. In some embodiments, an alkyl includes C₁-C₅ carbons. In some embodiments, an alkyl includes C₁-C₁₀ carbons. In some embodiments, an alkyl is a C₁-C₁₂ carbons. In some embodiments, an alkyl is a C₁-C₂₀ carbons. In some embodiments, branched alkyl is an alkyl substituted by alkyl side chains of 1 to 5 carbons. In various embodiments, the alkyl group may be unsubstituted. In some embodiments, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof.

The alkyl group can be a sole substituent, or it can be a component of a larger substituent, such as in an alkoxy, alkoxyalkyl, haloalkyl, arylalkyl, alkylamino, dialkylamino, alkylamido, alkylurea, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, 2, 3, or 4-CH₂—C₆H₄—Cl, C(OH)(CH₃)(Ph), etc.

As used herein, the term “aryl” refers to any aromatic ring that is directly bonded to another group and can be either substituted or unsubstituted. The aryl group can be a sole substituent, or the aryl group can be a component of a larger substituent, such as in an arylalkyl, arylamino, arylamido, etc. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, indolyl, phenylmethyl, phenylethyl, phenylamino, phenylamido, 3-methyl-4H-1,2,4-triazolyl, 5-methyl-1,2,4-oxadiazolyl, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, phenyl, halophenyl, (benzyloxy)phenyl, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O— alkyl, C(O)H, —C(O)NH₂ or any combination thereof.

As used herein, the term “alkoxy” refers to an ether group substituted by an alkyl group as defined above. Alkoxy refers both to linear and to branched alkoxy groups. Nonlimiting examples of alkoxy groups are methoxy, ethoxy, propoxy, iso-propoxy, tert-butoxy.

As used herein, the term “aminoalkyl” refers to an amine group substituted by an alkyl group as defined above. Aminoalkyl refers to monoalkylamine, dialkylamine or trialkylamine. Nonlimiting examples of aminoalkyl groups are —N(Me)₂, —NHMe, —NH₃.

A “haloalkyl” group refers, in some embodiments, to an alkyl group as defined above, which is substituted by one or more halogen atoms, e.g. by F, Cl, Br or I. The term “haloalkyl” include but is not limited to fluoroalkyl, i.e., to an alkyl group bearing at least one fluorine atom. Nonlimiting examples of haloalkyl groups are CF₃, CF₂CF₃, CF₂CH₃, CH₂CF₃, CF₂CH₂CH₃, CH₂CH₂CF₃, CF₂CH(CH₃)₂ and CF(CH₃)—CH(CH₃)₂.

A “halophenyl” group refers, in some embodiments, to a phenyl substitutent which is substituted by one or more halogen atoms, e.g. by F, Cl, Br or I. In one embodiment, the halophenyl is 4-chlorophenyl.

An “alkoxyalkyl” group refers, in some embodiments, to an alkyl group as defined above, which is substituted by alkoxy group as defined above, e.g. by methoxy, ethoxy, propoxy, i-propoxy, t-butoxy etc. Nonlimiting examples of alkoxyalkyl groups are —CH₂—O—CH₃, —CH₂—O—CH(CH₃)₂, —CH₂—O—C(CH₃)₃, —CH₂—CH₂—O—CH₃, —CH₂—CH₂—O—CH(CH₃)₂, —CH₂—CH₂—O—C(CH₃)₃.

A “cycloalkyl” or “carbocyclic” group refers, in various embodiments, to a ring structure comprising carbon atoms as ring atoms, which may be either saturated or unsaturated, substituted or unsubstituted, single or fused. In some embodiments the cycloalkyl is a 3-10 membered ring. In some embodiments the cycloalkyl is a 3-12 membered ring. In some embodiments the cycloalkyl is a 6 membered ring. In some embodiments the cycloalkyl is a 5-7 membered ring. In some embodiments the cycloalkyl is a 3-8 membered ring. In some embodiments, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof. In some embodiments, the cycloalkyl ring may be fused to another saturated or unsaturated cycloalkyl or heterocyclic 3-8 membered ring. In some embodiments, the cycloalkyl ring is a saturated ring. In some embodiments, the cycloalkyl ring is an unsaturated ring. Non limiting examples of a cycloalkyl group comprise cyclohexyl, cyclohexenyl, cyclopropyl, cyclopropenyl, cyclopentyl, cyclopentenyl, cyclobutyl, cyclobutenyl, cycloctyl, cycloctadienyl (COD), cycloctaene (COE) etc.

A “heterocycle” or “heterocyclic” group refers, in various embodiments, to a ring structure comprising in addition to carbon atoms, sulfur, oxygen, nitrogen or any combination thereof, as part of the ring. A “heteroaromatic ring” refers in various embodiments, to an aromatic ring structure comprising in addition to carbon atoms, sulfur, oxygen, nitrogen or any combination thereof, as part of the ring. In some embodiments the heterocycle or heteroaromatic ring is a 3-10 membered ring. In some embodiments the heterocycle or heteroaromatic ring is a 3-12 membered ring. In some embodiments the heterocycle or heteroaromatic ring is a 6 membered ring. In some embodiments the heterocycle or heteroaromatic ring is a 5-7 membered ring. In some embodiments the heterocycle or heteroaromatic ring is a 3-8 membered ring. In some embodiments, the heterocycle group or heteroaromatic ring may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof. In some embodiments, the heterocycle ring or heteroaromatic ring may be fused to another saturated or unsaturated cycloalkyl or heterocyclic 3-8 membered ring. In some embodiments, the heterocyclic ring is a saturated ring. In some embodiments, the heterocyclic ring is an unsaturated ring. Non limiting examples of a heterocyclic ring or heteroaromatic ring systems comprise pyridine, piperidine, morpholine, piperazine, thiophene, pyrrole, benzodioxole, benzofuran-2(3H)-one, benzo[d][1,3]dioxole, indole, oxazole, isoxazole, imidazole and 1-methylimidazole, furane, triazole, pyrimidine, pyrazine, oxacyclobutane (1 or 2-oxacyclobutane), naphthalene, tetrahydrothiophene 1,1-dioxide, thiazole, benzimidazole, piperidine, 1-methylpiperidine, isoquinoline, 1,3-dihydroisobenzofuran, benzofuran, 3-methyl-4H-1,2,4-triazole, 5-methyl-1,2,4-oxadiazole, or indole.

In various embodiments, this invention provides a compound of this invention or its isomer, metabolite, pharmaceutically acceptable salt, pharmaceutical product, tautomer, hydrate, N-oxide, reverse amide analog, prodrug, isotopic variant (deuterated analog), PROTAC, polymorph, or crystal or combinations thereof. In various embodiments, this invention provides an isomer of the compound of this invention. In some embodiments, this invention provides a metabolite of the compound of this invention. In some embodiments, this invention provides a pharmaceutically acceptable salt of the compound of this invention. In some embodiments, this invention provides a pharmaceutical product of the compound of this invention. In some embodiments, this invention provides a tautomer of the compound of this invention. In some embodiments, this invention provides a hydrate of the compound of this invention. In some embodiments, this invention provides an N-oxide of the compound of this invention. In some embodiments, this invention provides a reverse amide analog of the compound of this invention. In some embodiments, this invention provides a prodrug of the compound of this invention. In some embodiments, this invention provides an isotopic variant (including but not limited to deuterated analog) of the compound of this invention. In some embodiments, this invention provides a PROTAC (Proteolysis targeting chimera) of the compound of this invention. In some embodiments, this invention provides a polymorph of the compound of this invention. In some embodiments, this invention provides a crystal of the compound of this invention. In some embodiments, this invention provides composition comprising a compound of this invention, as described herein, or, In some embodiments, a combination of an isomer, metabolite, pharmaceutically acceptable salt, pharmaceutical product, tautomer, hydrate, N-oxide, reverse amide analog, prodrug, isotopic variant (deuterated analog), PROTAC, polymorph, or crystal of the compound of this invention.

In various embodiments, the term “isomer” includes, but is not limited to, stereoisomers and analogs, structural isomers and analogs, conformational isomers and analogs, and the like. In some embodiments, the isomer is an optical isomer. In some embodiments, the isomer is a stereoisomer.

In various embodiments, this invention encompasses the use of various stereoisomers of the compounds of the invention. It will be appreciated by those skilled in the art that the compounds of the present invention may contain at least one chiral center. Accordingly, the compounds used in the methods of the present invention may exist in, and be isolated in, optically-active or racemic forms. Accordingly, the compounds according to this invention may exist as optically-active isomers (enantiomers or diastereomers, including but not limited to: the (R), (S), (R)(R), (R)(S), (S)(S), (S)(R), (R)(R)(R), (R)(R)(S), (R)(S)(R), (S)(R)(R), (R)(S)(S), (S)(R)(S), (S)(S)(R) or (S)(S)(S) isomers); as racemic mixtures, or as enantiomerically enriched mixtures. Some compounds may also exhibit polymorphism. It is to be understood that the present invention encompasses any racemic, optically-active, polymorphic, or stereroisomeric form, or mixtures thereof, which form possesses properties useful in the treatment of the various conditions described herein.

It is well known in the art how to prepare optically-active forms (for example, by resolution of the racemic form by recrystallization techniques, by synthesis from optically-active starting materials, by chiral synthesis, or by chromatographic separation using a chiral stationary phase).

The compounds of the present invention can also be present in the form of a racemic mixture, containing substantially equivalent amounts of stereoisomers. In some embodiments, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 95% pure, more preferably at least about 98% pure, most preferably at least about 99% pure.

Compounds of the present invention can also be in the form of a hydrate, which means that the compound further includes a stoichiometric or non-stoichiometric amount of water bound by non-covalent intermolecular forces.

As used herein, when some chemical functional group (e.g. alkyl or aryl) is said to be “substituted”, it is herein defined that one or more substitutions are possible.

Compounds of the present invention may exist in the form of one or more of the possible tautomers and depending on the conditions it may be possible to separate some or all of the tautomers into individual and distinct entities. It is to be understood that all of the possible tautomers, including all additional enol and keto tautomers and/or isomers are hereby covered. For example, the following tautomers, but not limited to these, are included:

Tautomerization of the imidazole ring:

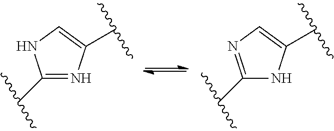 [SMILES: *c1c[nH]c(*)n1 ; *c1cnc(*)[nH]1]

Tautomerization of the pyrazolone ring:

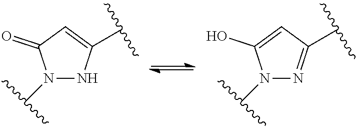 [SMILES: *c1cc(=O)n(*)[nH]1 ; *c1cc(O)n(*)n1]

The invention includes “pharmaceutically acceptable salts” of the compounds of this invention, which may be produced, by reaction of a compound of this invention with an acid or base. Certain compounds, particularly those possessing acid or basic groups, can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically acceptable salts of amines of compounds the compounds of this invention may be prepared from an inorganic acid or from an organic acid. In various embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In various embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In various embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

### Pharmaceutical Composition

Another aspect of the present invention relates to a pharmaceutical composition including a pharmaceutically acceptable carrier and a compound according to the aspects of the present invention. The pharmaceutical composition can contain one or more of the above-identified compounds of the present invention. Typically, the pharmaceutical composition of the present invention will include a compound of the present invention or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In some embodiments, these compounds are tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, these active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In various embodiments, the compounds of this invention are administered in combination with an agent treating fibrosis. In some embodiment, the agent treating lung fibrosis is at least one selected from: pirfenidone and Nintedanib. Other examples of agents which can be useful in treating lung fibrosis including IPF, in combination with compound of the invention, include but are not limited to: Pioglitazone, Tralokinumab, Lebrikizumab, FG-3019, Simtuzumab, STX-100, BMS-986020, Rituximab, Carbon Monoxide, Azithromycin, and Cotrimoxazole. In various embodiments, the compounds of this invention are administered in combination with an agent treating NASH.

When administering the compounds of the present invention, they can be administered systemically or, alternatively, they can be administered directly to a specific site where fibrosis is present. Thus, administering can be accomplished in any manner effective for delivering the compounds or the pharmaceutical compositions to the fibrotic cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

### Biological Activity

In various embodiments, the invention provides compounds and compositions, including any embodiment described herein, for use in any of the methods of this invention. In various embodiments, use of a compound of this invention or a composition comprising the same, will have utility in inhibiting, suppressing, enhancing or stimulating a desired response in a subject, as will be understood by one skilled in the art. In some embodiments, the compositions may further comprise additional active ingredients, whose activity is useful for the particular application for which the compound of this invention is being administered.

The invention relates to the treatment, inhibition and reduction of fibrosis, including lung and hepatic fibrosis. More specifically, embodiments of the invention provide compositions and methods useful for the treatment and inhibition of fibrotic disorders, lung fibrosis, idiopathic pulmonary fibrosis (IPF), hepato-fibrotic conditions associated with Non-Alcoholic Fatty Liver Disease (NAFLD) and Non-Alcoholic Steatohepatitis (NASH), employing the use of a compound according to this invention or a pharmaceutically acceptable salt thereof. In another embodiment, the human subject is afflicted with lung fibrosis. In another embodiment, the human subject is afflicted with idiopathic pulmonary fibrosis (IPF). In another embodiment, the human subject is afflicted with Non-Alcoholic Fatty Liver Disease (NAFLD). In another embodiment, the human subject is afflicted with Non-Alcoholic Steatohepatitis (NASH). In another embodiment, the human subject is not afflicted with Non-Alcoholic Steatohepatitis (NASH).

In various conditions, the formation of fibrotic tissue is characterized by the deposition of abnormally large amounts of collagen. The synthesis of collagen is also involved in a number of other pathological conditions. For example, clinical conditions and disorders associated with primary or secondary fibrosis, such as systemic sclerosis, graft-versus host disease (GVHD), pulmonary fibrosis and autoimmune disorders, are distinguished by excessive production of connective tissue, which results in the destruction of normal tissue architecture and function. These diseases can best be interpreted in terms of perturbations in cellular functions, a major manifestation of which is excessive collagen synthesis and deposition. The role of collagen in fibrosis has prompted attempts to develop drugs that inhibit its accumulation.

Excessive accumulation of collagen is the major pathologic feature in a variety of clinical conditions characterized by tissue fibrosis. These conditions include localized processes, as for example, pulmonary fibrosis and liver cirrhosis, or more generalized processes, like progressive systemic sclerosis. Collagen deposition is a feature of different forms of dermal fibrosis, which in addition to scleroderma, include localized and generalized morphea, keloids, hypertrophic scars, familial cutaneous collagenoma and connective tissue nevi of the collagen type. Recent advances in the understanding of the normal biochemistry of collagen have allowed us to define specific levels of collagen biosynthesis and degradation at which a pharmacologic intervention could lead to reduced collagen deposition in the tissues. Such compounds could potentially provide us with novel means to reduce the excessive collagen accumulation in diseases.

Accordingly, in various embodiments, this invention is directed to a method of treating, suppressing, reducing the severity, reducing the risk of developing or inhibiting fibrosis in a subject, comprising administering a compound according to this invention, to a subject suffering from fibrosis under conditions effective to treat, suppress, reduce the severity, reduce the risk of developing, or inhibit fibrosis in said subject. In some embodiments, the fibrosis is systemic. In some embodiments, the fibrosis is organ specific. In some embodiments, the fibrosis is a result of wound healing. In some embodiments, the fibrosis is a result of scarring. In some embodiments, the fibrosis is primary or secondary fibrosis. In some embodiments, the fibrosis is a result of systemic sclerosis, progressive systemic sclerosis, graft-versus host disease (GVHD), pulmonary fibrosis, autoimmune disorders, or any combination thereof; each represents a separate embodiment according to this invention. In another embodiment, the human subject is afflicted with lung fibrosis. In another embodiment, the human subject is afflicted with idiopathic pulmonary fibrosis (IPF). In some embodiments, the fibrosis is pulmonary fibrosis. In some embodiments, the subject has a liver cirrhosis. In some embodiments, the fibrosis is hepatic fibrosis, lung fibrosis or dermal fibrosis. In some embodiments, the dermal fibrosis is scleroderma. In some embodiments, the dermal fibrosis is a result of a localized or generalized morphea, keloids, hypertrophic scars, familial cutaneous collagenoma, connective tissue nevi of the collagen type, or any combination thereof; each represents a separate embodiment according to this invention. In some embodiments, the fibrosis results from tissue injury, inflammation, oxidative stress or any combination thereof; each represents a separate embodiment according to this invention. In some embodiments, the fibrosis is gingival fibromatosis. In some embodiments, the compound is a Collagen I translation inhibitor. In some embodiments, the compounds are selective to Collagen I. In some embodiments, the compounds are selective to Collagen IA. In some embodiments, the compounds are selective to Collagen IA1. In some embodiments, the compound is any one of the compounds listed in Table 1; each compound represents a separate embodiment according to this invention.

Human fibrotic diseases constitute a major health problem worldwide owing to the large number of affected individuals, the incomplete knowledge of the fibrotic process pathogenesis, the marked heterogeneity in their etiology and clinical manifestations, the absence of appropriate and fully validated biomarkers, and, most importantly, the current void of effective disease-modifying therapeutic agents. The fibrotic disorders encompass a wide spectrum of clinical entities including systemic fibrotic diseases such as systemic sclerosis (SSc), sclerodermatous graft vs. host disease, and nephrogenic systemic fibrosis, as well as numerous organ-specific disorders including radiation-induced fibrosis and cardiac, pulmonary, lung, liver, and kidney fibrosis. Although their causative mechanisms are quite diverse and, in several instances have remained elusive, these diseases share the common feature of an uncontrolled and progressive accumulation of fibrotic tissue in affected organs causing their dysfunction and ultimate failure. Despite the remarkable heterogeneity in the etiologic mechanisms responsible for the development of fibrotic diseases and in their clinical manifestations, numerous studies have identified activated myofibroblasts as the common cellular element ultimately responsible for the replacement of normal tissues with nonfunctional fibrotic tissue.

In various embodiments, this invention is directed to a method of treating, suppressing, reducing the severity, reducing the risk of developing or inhibiting systemic fibrotic disease in a subject, comprising administering a compound according to this invention, to a subject suffering from a systemic fibrotic disease under conditions effective to treat, suppress, reduce the severity, reduce the risk of developing, or inhibit the systemic fibrotic disease in said subject. In some embodiments, the systemic fibrotic disease is systemic sclerosis. In some embodiments, the systemic fibrotic disease is multifocal fibrosclerosis (IgG4-associated fibrosis). In some embodiments, the systemic fibrotic disease is nephrogenic systemic fibrosis. In some embodiments, the systemic fibrotic disease is sclerodermatous graft vs. host disease.

In various embodiments, this invention is directed to a method of treating, suppressing, reducing the severity, reducing the risk of developing or inhibiting an organ-specific fibrotic disease in a subject, comprising administering a compound according to this invention, to a subject suffering from an organ-specific fibrotic disease under conditions effective to treat, suppress, reduce the severity, reduce the risk of developing, or inhibit the organ-specific fibrotic disease in said subject.

In some embodiments, the organ-specific fibrotic disease is lung fibrosis. In some embodiments, the organ-specific fibrotic disease is idiopathic pulmonary fibrosis (IPF).

In some embodiments, the organ-specific fibrotic disease is cardiac fibrosis. In some embodiments, the cardiac fibrosis is hypertension-associated cardiac fibrosis. In some embodiments, the cardiac fibrosis is post-myocardial infarction. In some embodiments, the cardiac fibrosis is chagas disease-induced myocardial fibrosis.

In some embodiments, the organ-specific fibrotic disease is kidney fibrosis. In some embodiments, the kidney fibrosis is diabetic and hypertensive nephropathy. In some embodiments, the kidney fibrosis is urinary tract obstruction-induced kidney fibrosis. In some embodiments, the kidney fibrosis is inflammatory/autoimmune-induced kidney fibrosis. In some embodiments, the kidney fibrosis is aristolochic acid nephropathy. In some embodiments, the kidney fibrosis is polycystic kidney disease.

In various embodiments, this invention is directed to a method of treating, suppressing, reducing the severity, reducing the risk of developing or inhibiting cardiac fibrosis in a subject, comprising administering a compound of this invention, to a subject suffering from cardiac fibrosis under conditions effective to treat, suppress, reduce the severity, reduce the risk of developing, or inhibit cardiac fibrosis in said subject. In some embodiments, the compound is a Collagen I translation inhibitor. In some embodiments, the compound is any one of the compounds listed in Table 1; each compound represents a separate embodiment according to this invention.

In some embodiments, the organ-specific fibrotic disease is pulmonary fibrosis. In some embodiments, the pulmonary fibrosis is idiopathic pulmonary fibrosis. In some embodiments, the pulmonary fibrosis is silica-induced pneumoconiosis (silicosis). In some embodiments, the pulmonary fibrosis is asbestos-induced pulmonary fibrosis (asbestosis). In some embodiments, the pulmonary fibrosis is chemotherapeutic agent-induced pulmonary fibrosis.

In some embodiments, the organ-specific fibrotic disease is liver and portal vein fibrosis. In some embodiments, the liver and portal vein fibrosis is alcoholic and nonalcoholic liver fibrosis. In some embodiments, the liver and portal vein fibrosis is hepatitis C-induced liver fibrosis. In some embodiments, the liver and portal vein fibrosis is primary biliary cirrhosis. In some embodiments, the liver and portal vein fibrosis is parasite-induced liver fibrosis (schistosomiasis).

In some embodiments, the organ-specific fibrotic disease is radiation-induced fibrosis (various organs). In some embodiments, the organ-specific fibrotic disease is bladder fibrosis. In some embodiments, the organ-specific fibrotic disease is intestinal fibrosis. In some embodiments, the organ-specific fibrotic disease is peritoneal sclerosis.

In some embodiments, the organ-specific fibrotic disease is diffuse fasciitis. In some embodiments, the diffuse fasciitis is localized scleroderma, keloids. In some embodiments, the diffuse fasciitis is dupuytren's disease. In some embodiments, the diffuse fasciitis is peyronie's disease. In some embodiments, the diffuse fasciitis is myelofibrosis. In some embodiments, the diffuse fasciitis is oral submucous fibrosis.

In some embodiments, the organ-specific fibrotic disease is a result of wound healing. In some embodiments, the organ-specific fibrotic disease is a result of scarring.

Fibrosis of the liver, also referred to herein as hepatic fibrosis, may be caused by various types of chronic liver injury, especially if an inflammatory component is involved. Self-limited, acute liver injury (e.g., acute viral hepatitis A), even when fulminant, does not necessarily distort the scaffolding architecture and hence does not typically cause fibrosis, despite loss of hepatocytes. However, factors such as chronic alcoholism, malnutrition, hemochromatosis, and exposure to poisons, toxins or drugs, may lead to chronic liver injury and hepatic fibrosis due to exposure to hepatotoxic chemical substances. Hepatic scarring, caused by surgery or other forms of injury associated with mechanical biliary obstruction, may also result in liver fibrosis.

In various embodiments, this invention is directed to a method of treating, suppressing, reducing the severity, reducing the risk of developing or inhibiting hepatic fibrosis in a subject, comprising administering a compound of this invention, to a subject suffering from hepatic fibrosis under conditions effective to treat, suppress, reduce the severity, reduce the risk of developing, or inhibit hepatic fibrosis in said subject. In some embodiments, the hepatic fibrosis results from hepatic scarring. In some embodiments, the hepatic fibrosis results from chronic liver injury. In some embodiments, the chronic liver injury results from chronic alcoholism, malnutrition, hemochromatosis, exposure to poisons, toxins or drugs; each represents a separate embodiment according to this invention. In some embodiments, the subject has a liver cirrhosis. In some embodiments, the compound is a Collagen I translation inhibitor. In some embodiments, the compound is any one of the compounds listed in Table 1; each compound represents a separate embodiment according to this invention.

Fibrosis itself is not necessarily symptomatic, however it can lead to the development of portal hypertension, in which scarring distorts blood flow through the liver, or cirrhosis, in which scarring results in disruption of normal hepatic architecture and liver dysfunction. The extent of each of these pathologies determines the clinical manifestation of hepato-fibrotic disorders. For example, congenital hepatic fibrosis affects portal vein branches, largely sparing the parenchyma. The result is portal hypertension with sparing of hepatocellular function.

In various embodiments, this invention is directed to a method of treating, suppressing, reducing the severity, reducing the risk of developing or inhibiting an hepato-fibrotic disorder in a subject, comprising administering a compound of this invention, to a subject suffering from hepato-fibrotic disorder under conditions effective to treat, suppress, reduce the severity, reduce the risk of developing, or inhibit the hepato-fibrotic disorder in said subject. In some embodiments, the hepato-fibrotic disorder is: portal hypertension, cirrhosis, congenital hepatic fibrosis or any combination thereof; each represents a separate embodiment according to this invention. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting portal hypertension in a subject, comprising administering a compound of this invention, to a subject suffering from portal hypertension under conditions effective to treat, suppress, reduce the severity, reduce the risk of developing, or inhibit portal hypertension in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting cirrhosis in a subject, comprising administering a compound of this invention, to a subject suffering from cirrhosis under conditions effective to treat, suppress, reduce the severity, reduce the risk of developing, or inhibit cirrhosis in said subject. In some embodiments, the cirrhosis is a result of hepatitis. In some embodiments, the cirrhosis is a result of alcoholism. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting human alcoholism in a subject, comprising administering a compound of this invention, to a subject suffering from alcoholism under conditions effective to treat, suppress, reduce the severity, reduce the risk of developing, or inhibit alcoholism in said subject. In some embodiments, the compound is a Collagen I translation inhibitor. In some embodiments, the compound is any one of the compounds listed in Table 1; each compound represents a separate embodiment according to this invention.

Non-alcoholic steatohepatitis (NASH) and alcoholic steatohepatitis (ASH) have a similar pathogenesis and histopathology but a different etiology and epidemiology. NASH and ASH are advanced stages of non-alcoholic fatty liver disease (NAFLD) and alcoholic fatty liver disease (AFLD). NAFLD is characterized by excessive fat accumulation in the liver (steatosis), without any other evident causes of chronic liver diseases (viral, autoimmune, genetic, etc.), and with an alcohol consumption ≤20-30 g/day. On the contrary, AFLD is defined as the presence of steatosis and alcohol consumption >20-30 g/day.

In various embodiments, this invention is directed to a method of treating, suppressing, reducing the severity, reducing the risk of developing or inhibiting Non-alcoholic steatohepatitis (NASH) in a subject, comprising administering a compound of this invention, to a subject suffering from Non-alcoholic steatohepatitis (NASH) under conditions effective to treat, suppress, reduce the severity, reduce the risk of developing, or inhibit Non-alcoholic steatohepatitis (NASH) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting alcoholic steatohepatitis (ASH) in a subject, comprising administering a compound of this invention, to a subject suffering from alcoholic steatohepatitis (ASH) under conditions effective to treat, suppress, reduce the severity, reduce the risk of developing, or inhibit alcoholic steatohepatitis (ASH) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting non-alcoholic fatty liver disease (NAFLD) in a subject, comprising administering a compound of this invention, to a subject suffering from non-alcoholic fatty liver disease (NAFLD) under conditions effective to treat, suppress, reduce the severity, reduce the risk of developing, or inhibit non-alcoholic fatty liver disease (NAFLD) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting alcoholic fatty liver disease (AFLD) in a subject, comprising administering a compound of this invention, to a subject suffering from alcoholic fatty liver disease (AFLD) under conditions effective to treat, suppress, reduce the severity, reduce the risk of developing, or inhibit alcoholic fatty liver disease (AFLD) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting lung fibrosis in a subject, comprising administering a compound of this invention, to a subject suffering from lung fibrosis under conditions effective to treat, suppress, reduce the severity, reduce the risk of developing, or inhibit lung fibrosis in said subject. In some embodiments, the compound is a Collagen I translation inhibitor. In some embodiments, the compound is any one of the compounds listed in Table 1; each compound represents a separate embodiment according to this invention.

Idiopathic pulmonary fibrosis (IPF) is an aging-associated recalcitrant lung disease with historically limited therapeutic options. The recent approval of two drugs, pirfenidone and nintedanib, by the United States Food and Drug Administration (FDA) in 2014 has heralded a new era in its management. Both drugs demonstrated efficacy in Phase III clinical trials by retarding the rate of progression of IPF; neither drug appears to be able to completely arrest disease progression. Advances in the understanding of IPF pathobiology have led to an unprecedented expansion in the number of potential therapeutic targets. Drugs targeting several of these are under investigation in various stages of clinical development.

In various embodiments, this invention is directed to a method of treating, suppressing, reducing the severity, reducing the risk of developing or inhibiting idiopathic pulmonary fibrosis (IPF) in a subject, comprising administering a compound of this invention, to a subject suffering from idiopathic pulmonary fibrosis (IPF) under conditions effective to treat, suppress, reduce the severity, reduce the risk of developing, or inhibit idiopathic pulmonary fibrosis (IPF) in said subject. In some embodiments, the compound is a Collagen I translation inhibitor. In some embodiments, the compound is any one of the compounds listed in Table 1; each compound represents a separate embodiment according to this invention. In some embodiments, the compound is administered in combination with an agent treating IPF. In some embodiments, the compound is administered in combination with pirfenidone, nintedanib, or combination thereof; each represents a separate embodiment according to this invention.

In various embodiments, this invention is directed to a method of treating, suppressing, reducing the severity, reducing the risk of developing or inhibiting dermal fibrosis in a subject, comprising administering a compound of this invention, to a subject suffering from dermal fibrosis under conditions effective to treat, suppress, reduce the severity, reduce the risk of developing, or inhibit dermal fibrosis in said subject. In some embodiments, the dermal fibrosis is scleroderma. In some embodiments, the dermal fibrosis is a result of a localized or generalized morphea, keloids, hypertrophic scars, familial cutaneous collagenoma, connective tissue nevi of the collagen type, or any combination thereof; each represents a separate embodiment according to this invention. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting scleroderma in a subject, comprising administering a compound of this invention, to a subject suffering from scleroderma under conditions effective to treat, suppress, reduce the severity, reduce the risk of developing, or inhibit scleroderma in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of inhibiting Collagen I (Col I) over production in a subject, comprising administering a compound of this invention, to a subject suffering from Collagen I (Col I) over production under conditions effective to inhibit Collagen I (Col I) over production in said subject. In some embodiments, the compound is a Collagen I translation inhibitor. In some embodiments, the compounds are Collagen I, II, II, IV, or V translation inhibitors; each represents a separate embodiment according to this invention. In some embodiments, the compounds are selective to Collagen I, II, II, IV, or V; each represents a separate embodiment according to this invention. In some embodiments, the compounds are selective to Collagen I. In some embodiments, the compounds are selective to Collagen IA. In some embodiments, the compounds are selective to Collagen IA1. In some embodiments, the compound is any one of the compounds listed in Table 1; each compound represents a separate embodiment according to this invention.

In some embodiments, this invention is directed to a method of treating, suppressing, reducing the severity, reducing the risk of developing or inhibiting an autoimmune disease or disorder in a subject, comprising administering a compound of this invention, to a subject suffering from an autoimmune disease or disorder under conditions effective to treat, suppress, reduce the severity, reduce the risk of developing, or inhibit the autoimmune disease or disorder in said subject. In some embodiments, the compound is a Collagen I translation inhibitor. In some embodiments, the compound is any one of the compounds listed in Table 1; each compound represents a separate embodiment according to this invention.

As used herein, subject or patient refers to any mammalian patient, including without limitation, humans and other primates, dogs, cats, horses, cows, sheep, pigs, rats, mice, and other rodents. In various embodiments, the subject is male. In some embodiments, the subject is female. In some embodiments, while the methods as described herein may be useful for treating either males or females.

The following examples are presented in order to more fully illustrate the preferred embodiments of the invention. They should in no way, however, be construed as limiting the broad scope of the invention.

### EXAMPLES

General

All compounds were profiled for cellular potency in inhibition of collagen 1 (COL1) protein translation using a phenotypic screening platform.

### Example 1

### Synthetic Details for Compounds of the Invention (Schemes 1-48)

### General Methods

All reagents were commercial grade and were used as received without further purification, unless otherwise specified. Reagent grade solvents were useds in all cases, unless otherwise specified. Thin layer chromatography was carried out using pre-coated silica gel F-254 plates (thickness 0.25 mm). ¹H-NMR and ¹⁹F-NMR spectra were recorded on a Bruker Bruker Avance 400 MHz or Avance III 400 MHz spectrometer. The chemical shifts are expressed in ppm using the residual solvent as internal standard. Splitting patterns are designated as s (singlet), d (doublet), dd (doublet of doublets), t (triplet), dt (doublet of triplets), q (quartet), m (multiplet) and br s (broad singlet).

### Abbreviations

AcOH Acetic acid
amphos Bis(di-tert-butyl(4-dimethylaminophenyl)phosphine
Boc tert-Butyloxycarbonyl
BuLi n-butyllithium
t-BuLi tert-butyllithium

### CDI 1,1′-Carbonyldiimidazole

DBU 1,8-Diazabicyclo[5.4.0]undec-7-ene
dppb 1,4-Bis(diphenylphosphino)butane
dppf 1,1′-Bis(diphenylphosphino)ferrocene

### DCM Dichloromethane

### DCE 1,2-Dichloroethane

DEAD Diethyl azodicarboxylate
DIAD Diisopropyl azodicarboxylate
DIBAL-H Diisobutylaluminum hydride

### DIPEA N,N-Diisopropylethylamine

### DMF N,N-Dimethylformamide

### DMA N,N-Dimethylacetamide

### DMAP 4-Dimethylaminopyridine

### DME 1,2-Dimethoxyethane

### DMSO Dimethylsulfoxide

EDC.HCl N-(3-Dimethylaminopropyl)-N′-ethylcarbodiimide hydrochloride
HATU [0-(7-Azabenzotriazol-1-yl)-N,N,N′,N′-tetramethyluronium-hexafluorophosphate]
HPLC High performance liquid chromatography
MsCl Methanesulfonyl chloride

### NBS N-Bromosuccinimide

NMP N-Methyl-2-pyrrolidinone
rt Room temperature

### SEM 2-(Trimethylsilyl)ethoxymethyl

T3P Propylphosphonic anhydride
TBAF Tetrabutylammonium fluoride
TBDMS tert-Butyldimethylsilyl
TBDPS tert-Butyldiphenylsilyl
TCFH N,N,N′,N′-tetramethylchloroformamidinium hexafluorophosphate

### THF Tetrahydrofuran

TMS-OTf Trimethylsilyl trifluoromethanesulfonate

### General Synthesis of Compounds of the Invention

### RHS Modifications

The two-step synthetic sequence towards the RHS-modified analogues of Compound 300 (see Table 1 for structures) is shown in Scheme 1.

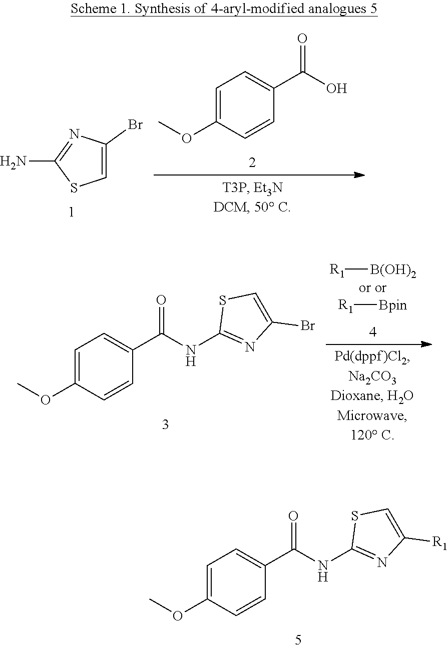

The first step of the synthesis involved an amide coupling reaction of 4-bromothiazol-2-amine 1 with 4-methoxybenzoic acid 2 in the presence of propylphosphonic anhydride (T3P) at elevated temperature affording intermediate 3. The second and final step was a Suzuki coupling under microwave conditions at 120° C. using intermediate 3. A variety of different aryl boronic acids or pinacol esters 4 were used, using [1,1′-bis(diphenylphosphino)ferrocene] dichloropalladium(II) as the catalyst in the presence of sodium carbonate as the base, in a mixture of dioxane and water to deliver the final compounds 5.

A similar synthetic sequence to Scheme 1 above was employed to synthesize 4-aryl-modified analogues of 6-methyl nicotinamides outlined in Scheme 2.

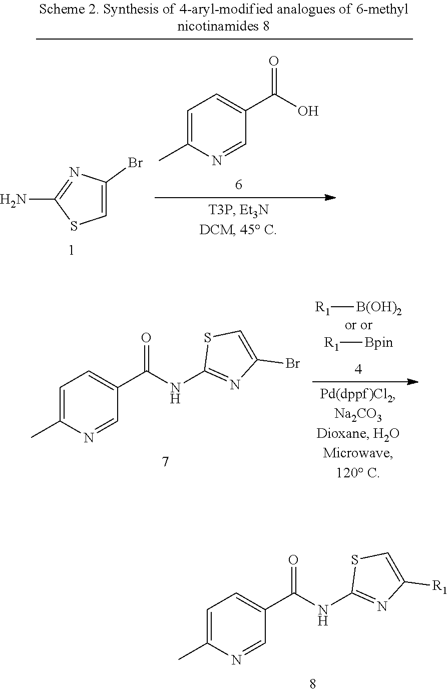

4-Bromothiazol-2-amine 1 was coupled with 6-methylnicotinic acid 6 in the presence of propylphosphonic anhydride (T3P) and triethylamine providing amide intermediate 7. The RHS aryl moieties were then introduced via Suzuki coupling of intermediate 7 with various boronic acids or pinacol esters 4 affording the desired final compounds 8.
The general synthesis scheme towards RHS-modified analogues of 2-methoxypyrimidine-5-carboxamides is shown in Scheme 3.

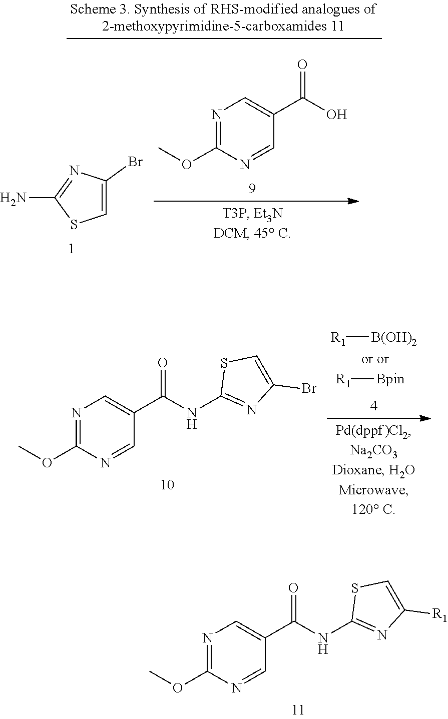

4-Bromothiazol-2-amine 1 was coupled with 2-methoxypyrimidine-5-carboxylic acid 9 in the presence of propylphosphonic anhydride (T3P) and triethylamine providing amide intermediate 10. The RHS aryl groups were introduced via Suzuki coupling of intermediate 10 with several boronic acids or pinacol esters 4 providing the desired final compounds 11.

The general synthesis scheme towards the RHS-modified analogues of Compound 327 (see Table 1 for structures) is outlined in Scheme 4.

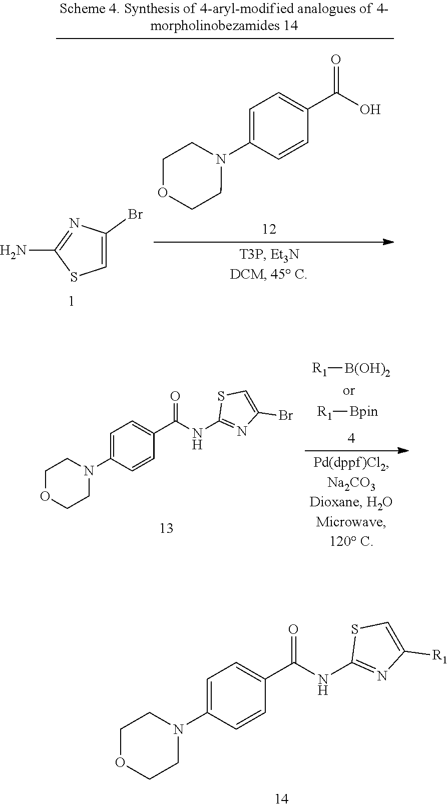

4-Bromothiazol-2-amine 1 was coupled with 4-morpholinobenzoic acid 12 in the presence of propylphosphonic anhydride (T3P) and triethylamine providing amide intermediate 13. The RHS aryl moieties were introduced via Suzuki coupling of intermediate 13 with various boronic acids or pinacol esters 4 affording the desired target compounds 14.

An alternative synthetic route was adopted to prepare the RHS 2-pyridyl analogue compound 19 (Compound 370), which is shown in Scheme 5.

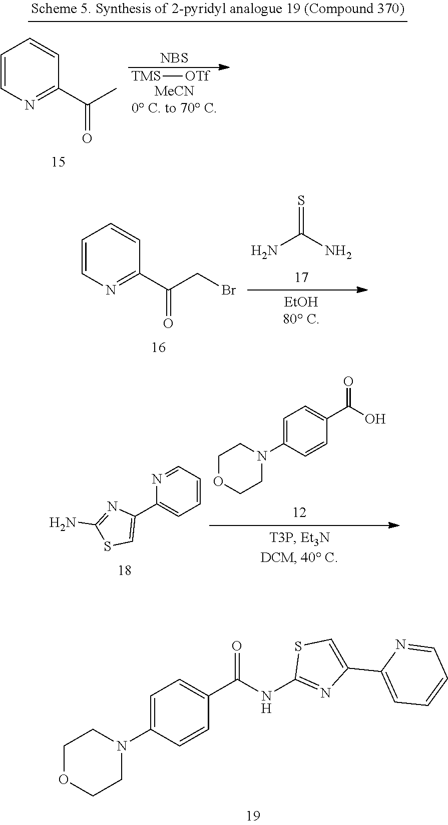

Commercial 2-acylpyridine 15 was brominated at the alpha position of the keto function, by using N-bromosuccinimide in the presence of TMS triflate in acetonitrile providing α-bromo ketone intermediate 16. Intermediate 16 was then heated at reflux in ethanol with thiourea 17 affording intermediate aminothiazole 18. Final step amide coupling was achieved by reacting aminotriazole 18 with 4-morpholinobenzoic acid 12 in the presence of propylphosphonic anhydride (T3P) and triethylamine to afford the 2-pyridyl final compound 19.

### LHS Modifications

The synthetic route towards the LHS amide analogues of Compounds 300 and 304 is shown in Scheme 6.

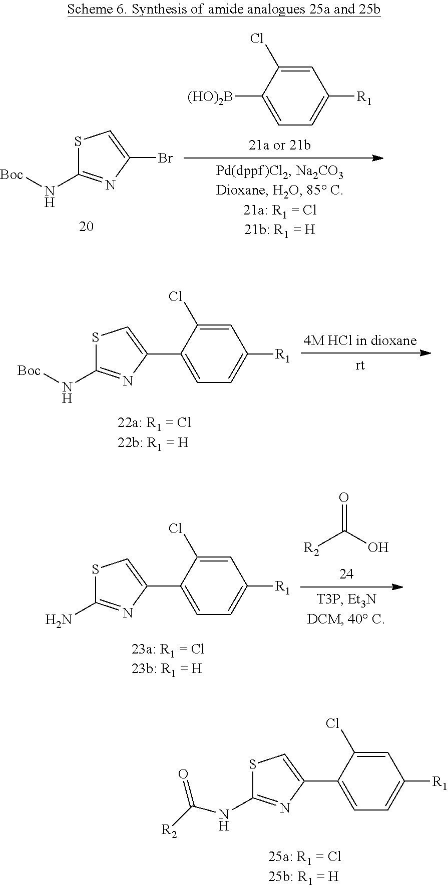

N-Boc 4-bromothiazol-2-amine 20 was subjected to a Suzuki reaction, using boronic acid 21a or 21b, in the presence of [1,1′-bis(diphenylphosphino)ferrocene] dichloropalladium(II) and sodium carbonate in dioxane and water to afford intermediate 22a or 22b. Removal of the N-Boc protecting group was achieved by treating 22a or 22b with a 4 M solution of HCl in dioxane affording aminothiazole 23a or 23b. Key aminothiazole intermediate 23a or 23b were converted to the target amides 25a or 25b using carboxylic acids 24 and the T3P protocol.

Urea based analogues were synthesized as detailed in Scheme 7.

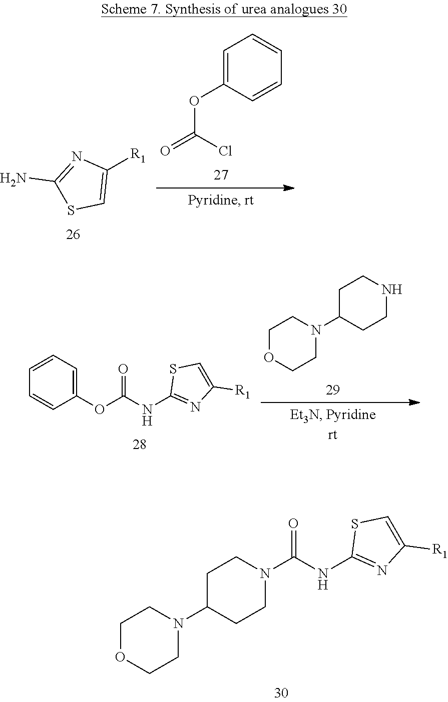

The aminothiazole intermediates 26 were converted to the activated carbamate intermediates 28 by treatment with phenylchloroformate 26 in pyridine at room temperature. The carbamate intermediates 28 were then reacted with 4-(piperidin-4-yl)morpholine 29 in the presence of triethylamine and pyridine affording the target urea analogues 30.

Piperazine sulphonamide analogues were accessed by the synthetic route shown in Scheme 8.

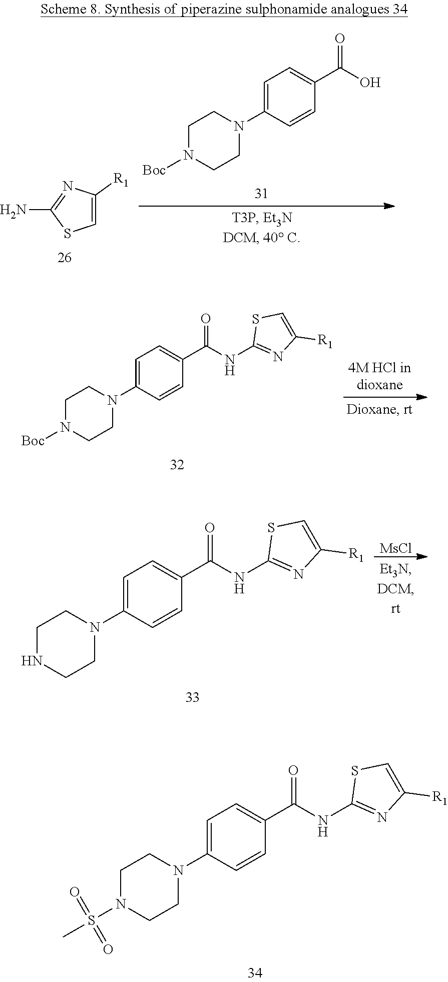

Starting aminothiazoles 26 were coupled with 4-(4-(tert-butoxycarbonyl)piperazin-1-yl)benzoic acid 31, using the previously described T3P protocol to afford the intermediate amides 32. N-Boc deprotection of these N-Boc protected piperazine amide intermediates 32 with a 4 M solution of HCl in dioxane gave the amine intermediates 33 as the free base following basic aqueous work-up. Sulfonylation of the piperazine intermediates 33 with methanesulfonyl chloride, in the presence of triethylamine in DCM afforded the desired piperazine sulfonamides 34.

The synthesis of two non-commercial morpholino carboxylic acids is summarised in Scheme 9.

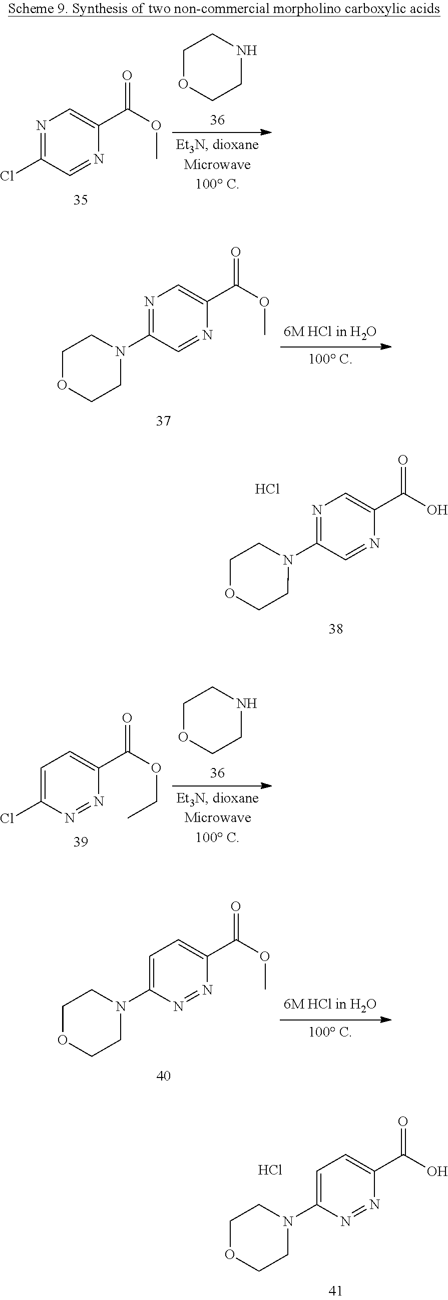

Aromatic substitution of commercial chloro pyrazine and pyridazine esters 35 and 39 with morpholine 36 in the microwave at 100° C. enabled access to the morpholino ester intermediates 37 and 40 respectively. These intermediates 37 and 40 were each then refluxed in 6 M aqueous hydrochloric acid affording the morpholino carboxylic acids 38 and 41 respectively as hydrochloride salts.

### Scaffold Modifications

The synthesis of the oxazole analogue 45 is shown in Scheme 10.

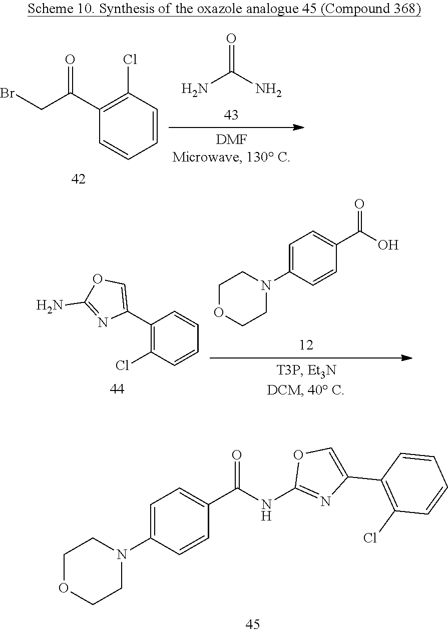

Commercial 2-chlorophenyl α-bromoketone 42 was heated with urea 43 in DMF in a microwave at 130° C. to afford the cyclised aminooxazole 44. The final amide step coupling of aminooxazole intermediate 43 with 4-morpholinobenzoic acid 12, using the propylphosphonic anhydride (T3P) protocol gave the desired target 45 (Compound 368).

A general synthesis of combination analogues combining a RHS aryl/heteroaryl group with morpholino-heteroaryl LHS moieties is described in Scheme 11 (see Table 1 for structures).

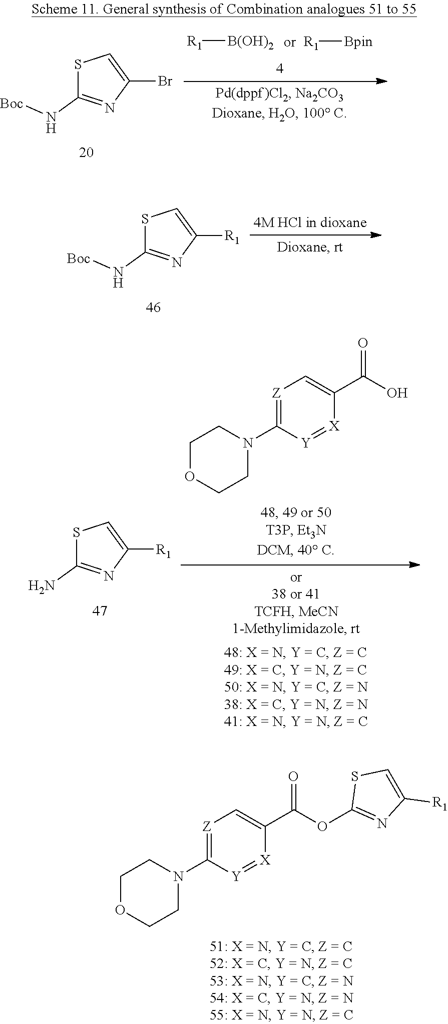

Suzuki coupling of N-Boc 4-bromothiazol-2-amine 20 with a variety of boronic acids or pinacol esters 4, using [1,1′-bis(diphenylphosphino)ferrocene] dichloropalladium(II) and sodium carbonate in dioxane and water at 100° C. enabled the synthesis of N-Boc aminothiazole intermediates 46.

Removal of the N-Boc protecting group was achieved by treating the N-Boc aminothiazole intermediates 46 with a 4 M solution of HCl in dioxane affording aminothiazole intermediates 47 as the free base, following basic aqueous work-up. Key aminothiazole intermediates 47 were converted to the target amides 51 to 55 using one of two amide coupling protocols. While for free base morpholino carboxylic acids of type 48, 49 and 50, the T3P protocol was used to prepare the target amide analogues 51, 52 and 53. The hydrochloride salts of 38 and 41 were successfully converted to the final amide targets 54 and 55 respectively, using the TCFH coupling reagent in the presence of 1-methylimidazole.

The three-step synthesis of compound 60 (Compound 376) is outlined in Scheme 12.

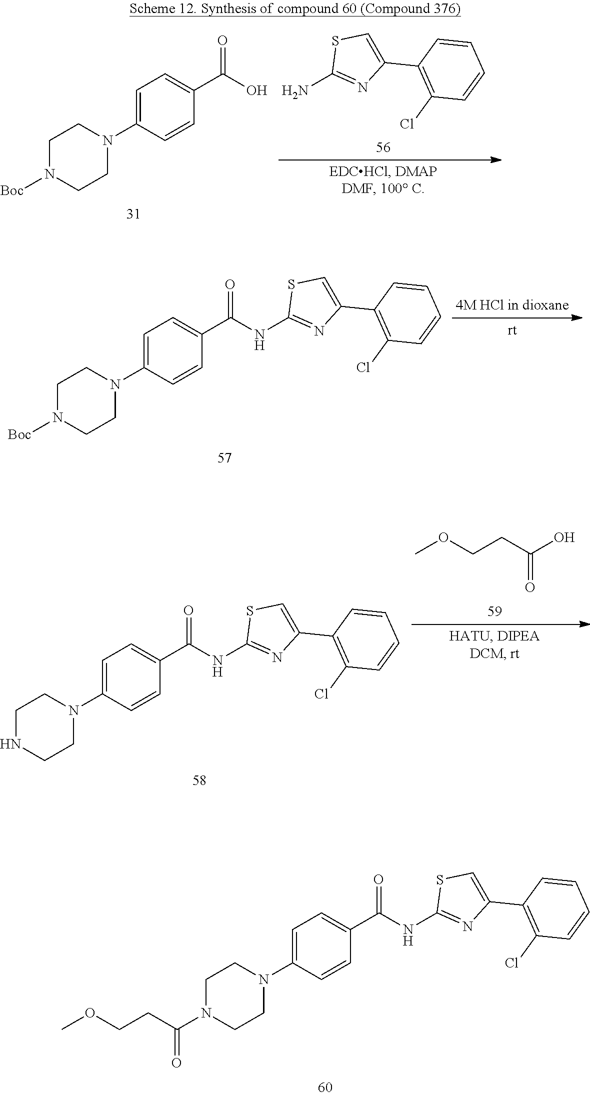

The first step involved amide coupling of 4-(4-(tert-butoxycarbonyl)piperazin-1-yl)benzoic acid 31 with 4-(2-chlorophenyl)thiazol-2-amine 56, using N-(3-dimethylaminopropyl)-N′-ethylcarbodiimide hydrochloride with DMAP in DMF at 100° C. to deliver intermediate 57 in good yield. This amide coupling protocol facilitated scale-up and was an alternative approach to the T3P protocol described in Scheme 8. Removal of the N-Boc protecting group was achieved by treating intermediate 57 with a 4 M solution of HCl in dioxane affording aminothiazole intermediate 58. Final step amide coupling of aminothiazole intermediate 58 with 3-methoxypropanoic acid 59, using HATU coupling conditions afforded the final compound 60 (Compound 376).

The single step synthesis of compound 62 (Compound 377) from intermediate 58 is outlined in Scheme 13.

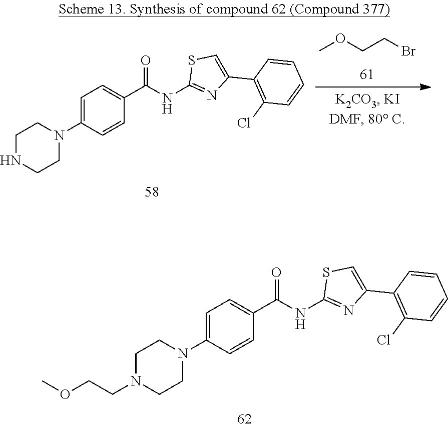

Piperazine intermediate 58 was N-alkylated with 2-bromoethyl methyl ether 61, in the presence of potassium carbonate and potassium iodide in DMF at 80° C. to synthesize readily final compound 62 (Compound 377).

The single step synthesis of compounds 64 and 66 (Compound 378 and 379) is shown in Scheme 14.

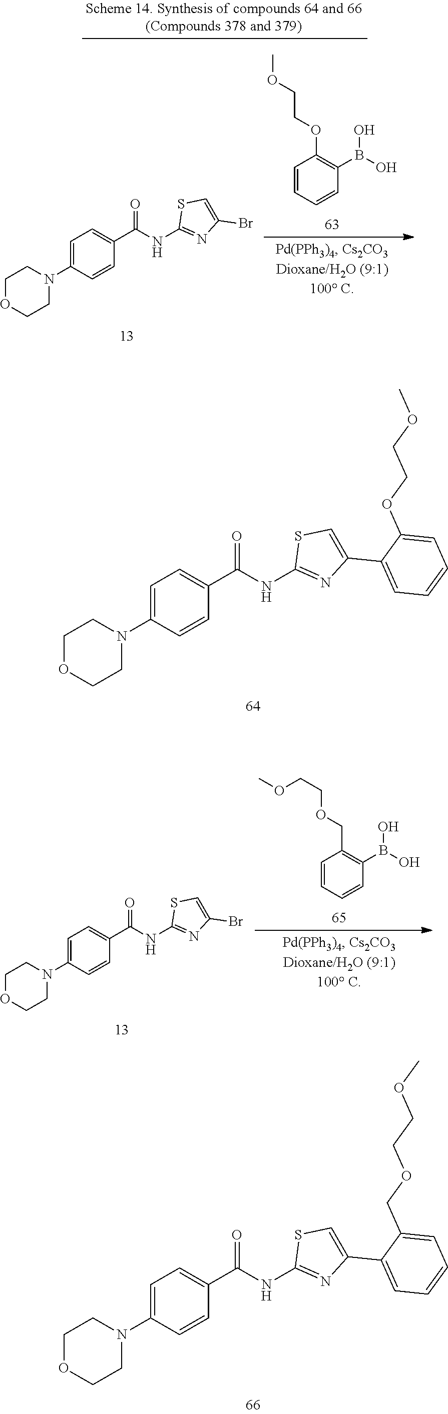 [SMILES: O=C(Nc1nc(Br)cs1)c1ccc(N2CCOCC2)cc1 ; COCCOc1ccccc1B(O)O ; COCCOc1ccccc1-c1csc(NC(=O)c2ccc(N3CCOCC3)cc2)n1 ; O=C(Nc1nc(Br)cs1)c1ccc(N2CCOCC2)cc1 ; COCCOCc1ccccc1B(O)O ; COCCOCc1ccccc1-c1csc(NC(=O)c2ccc(N3CCOCC3)cc2)n1]

Conventionally heated Suzuki coupling of bromo thiazole intermediate 13 with (2-(2-methoxyethoxy)phenyl)boronic acid 63, using tetrakis(triphenylphosphine)palladium(0) and cesium carbonate in dioxane and water at 100° C. enabled the synthesis of compound 64 (Compound 378).

The same conventionally heated Suzuki coupling protocol was used with 2-[(2-methoxyethoxy)methyl]phenylboronic acid 65 to afford final compound 66 (Compound 379).

The single step synthesis of compounds 69 and 70 (Compounds 399 and 400) (see Table 1 for structures) is shown in Scheme 15.

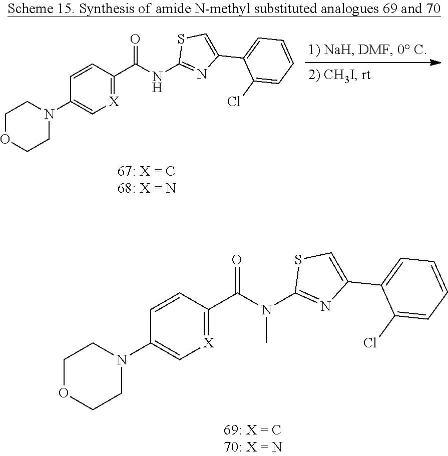

The intermediate amides 67 and 68 underwent N-alkylation using sodium hydride in DMF as a base at 0° C. followed by the addition of iodomethane at room temperature to deliver the final compounds 69 and 70 (Compounds 399 and 400).

A general synthesis of bis-amide analogues combining a RHS aryl/heteroaryl group with various amine LHS moieties is described in Scheme 16 (see Table 1 for structures).

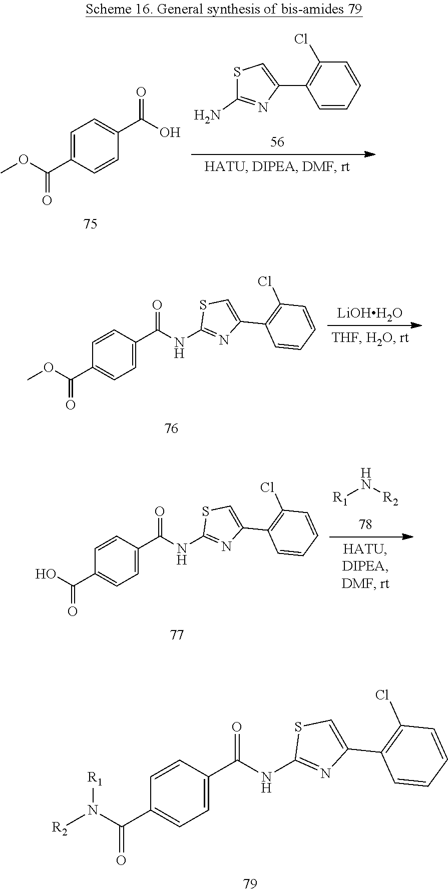

The first step of the synthesis involved an amide coupling reaction of 4-(2-chlorophenyl)thiazol-2-amine 56 with 4-(methoxycarbonyl)benzoic acid 75 in the presence of HATU and DIPEA in DMF affording intermediate 76. The second step involved hydrolysis of methyl ester intermediate 76 using lithium hydroxide to deliver the carboxylic acid intermediate 77. The final synthetic step of the sequence was an amide coupling reaction employing similar reaction conditions to step 1. A variety of different amines 78 were used to deliver the final bis-amide compounds 79.

A similar synthetic sequence to Scheme 17 above was employed to synthesize compound 84 (Compound 430) as outlined in Scheme 17.

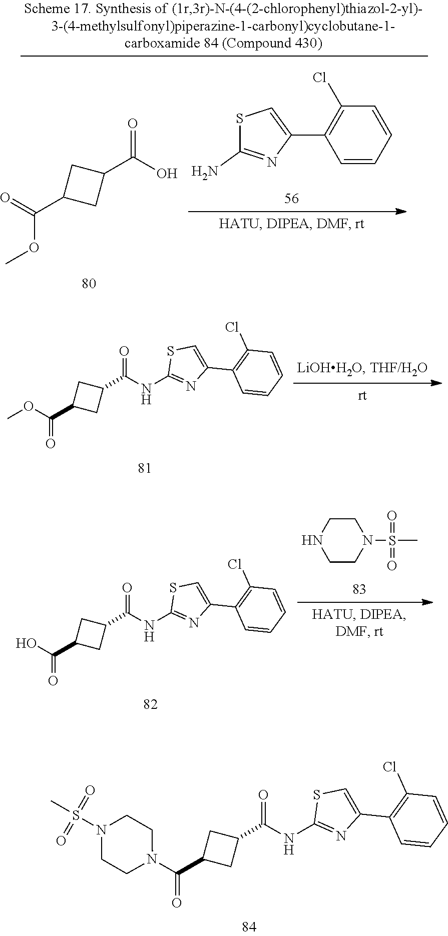

4-(2-Chlorophenyl)thiazol-2-amine 56 and 3-(methoxycarbonyl)cyclobutane-1-carboxylic acid 80 underwent a HATU mediated amide coupling reaction to generate amide 81. Methyl ester intermediate 81 was hydrolyzed using lithium hydroxide in aqueous THF to afford carboxylic acid intermediate 82. The final step involved HATU mediated amide coupling of carboxylic acid 82 with amine 83 to deliver the final compound 84 (Compound 430).

The piperazine sulfonamide analogue compound 91 (Compound 403) was accessed by the synthetic route shown in Scheme 18.

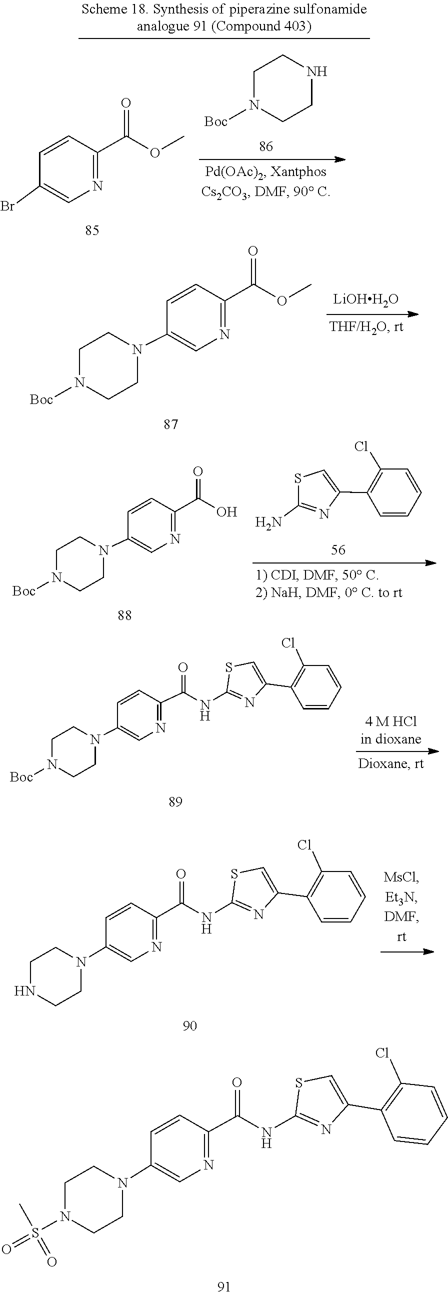

The first step of the synthetic sequence involved a Buchwald C—N coupling reaction between methyl 5-bromopicolinate 85 and tert-butyl piperazine-1-carboxylate 86 to generate the methyl ester intermediate 87. Hydrolysis of the intermediate methyl ester using lithium hydroxide in aqueous THF at ambient temperature afforded the carboxylic acid intermediate 88. Intermediate 88 was subjected to two-step amide coupling reaction to generate amide intermediate 89. First of all, the acyl imidazole activated intermediate of carboxylic acid 88 was generated using CDI in DMF at 50° C. Subsequently, the acyl imidazole activated intermediate was then reacted with 4-(2-chlorophenyl)thiazol-2-amine 56 in the presence of sodium hydride at 0° C. to enable amide formation. Removal of the N-Boc protecting group was achieved by treating intermediate 89 with a 4 M solution of HCl in dioxane affording intermediate piperazine 90 as a hydrochloride salt. Sulfonylation of the key piperazine intermediate 90 with methanesulfonyl chloride, in the presence of triethylamine in DMF afforded the desired piperazine sulfonamides 91 (Compound 403).

An alternative synthesis for the preparation of key piperazine intermediate 90 is shown in Scheme 19.

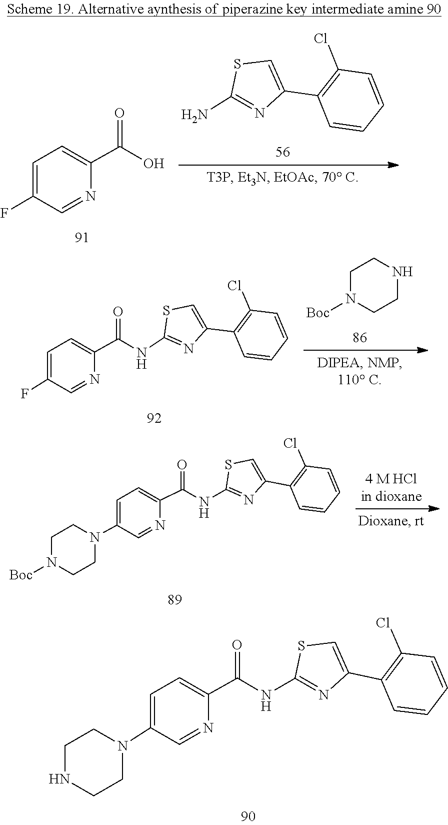 [SMILES: O=C(O)c1ccc(F)cn1 ; Nc1nc(-c2ccccc2Cl)cs1 ; O=C(Nc1nc(-c2ccccc2Cl)cs1)c1ccc(F)cn1 ; CC(C)(C)OC(=O)N1CCNCC1 ; CC(C)(C)OC(=O)N1CCN(c2ccc(C(=O)Nc3nc(-c4ccccc4Cl)cs3)nc2)CC1 ; O=C(Nc1nc(-c2ccccc2Cl)cs1)c1ccc(N2CCNCC2)cn1]

Amide intermediate 92 was generated by reacting 4-(2-chlorophenyl)thiazol-2-amine 56 and 5-fluoropicolinic acid 91 in the presence of propylphosphonic anhydride (T3P) and triethylamine in ethyl acetate at elevated temperature. In this synthetic route, intermediate 89 was generated by a nucleophilic aromatic substitution reaction of 92 with tert-butyl piperazine-1-carboxylate 86 using DIPEA, as the base in NMP at 110° C. As described for Scheme 19, the N-Boc protecting group was removed under acidic conditions to afford the same piperazine intermediate 90 as a hydrochloride salt.

A similar synthetic sequence to Scheme 18 above was employed to synthesize the 2-substituted 2,7-diazaspiro[3.5]nonane intermediate 96 as outlined in Scheme 20.

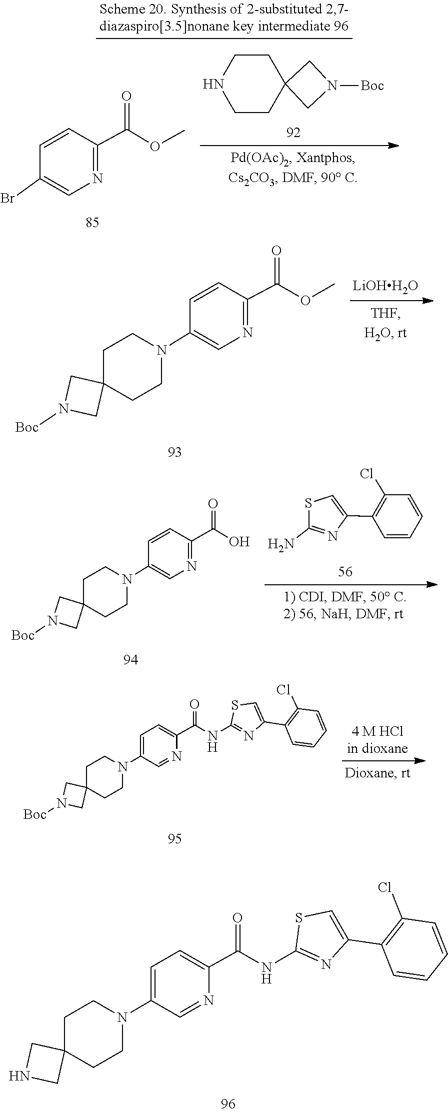

The first step of the synthetic sequence involved Buchwald C—N coupling reaction between methyl 5-bromopicolinate 85 and tert-butyl 2,7-diazaspiro[3.5]nonane-2-carboxylate 92 to generate intermediate methyl ester 93. Hydrolysis of the intermediate methyl ester using lithium hydroxide in aqueous THF at ambient temperature afforded the carboxylic acid intermediate 94. Intermediate 93 was was subjected to a two-step amide coupling reaction to generate amide intermediate 95. First of all, the acyl imidazole activated intermediate of carboxylic acid 94 was generated using CDI in DMF at 50° C. Subsequently, the acyl imidazole activated intermediate was then reacted with 4-(2-chlorophenyl)thiazol-2-amine 56 in the presence of sodium hydride at 0° C. to enable amide formation. Removal of the N-Boc protecting group was achieved by treating intermediate 95 with a 4 M solution of HCl in dioxane affording intermediate substituted 2,7-diazaspiro[3.5]nonane 96 as a hydrochloride salt.

A similar synthetic sequence to Scheme 19 above was employed to synthesize 2-substituted 2,6-diazaspiro[3.3]heptane intermediate 99 as shown in Scheme 21.

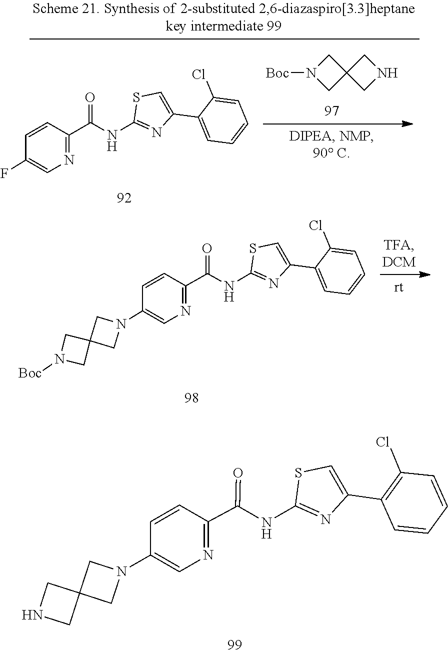 [SMILES: O=C(Nc1nc(-c2ccccc2Cl)cs1)c1ccc(F)cn1 ; CC(C)(C)OC(=O)N1CC2(CNC2)C1 ; CC(C)(C)OC(=O)N1CC2(C1)CN(c1ccc(C(=O)Nc3nc(-c4ccccc4Cl)cs3)nc1)C2 ; O=C(Nc1nc(-c2ccccc2Cl)cs1)c1ccc(N2CC3(CNC3)C2)cn1]

In this synthetic route, intermediate 98 was generated by a nucleophilic aromatic substitution reaction of 92 with tert-butyl 2,6-diazaspiro[3.3]heptane-2-carboxylate 97 using DIPEA as the base in NMP at 110° C. Removal of the N-Boc protecting group was achieved by treating intermediate 98 with trifluoroacetic acid in DCM at room temperature. The substituted 2,6-diazaspiro[3.3]heptane key intermediate 99 was generated as a trifluoroacetate salt.

The synthesis of an O-linked piperidine intermediate 105 is outlined in Scheme 22.

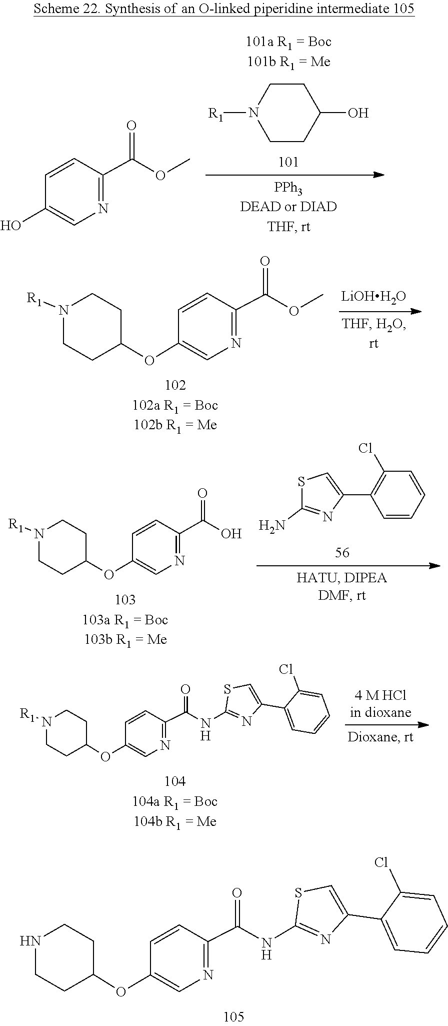

The first step of the synthesis involved a Mitsunobu reaction between methyl 5-hydroxypicolinate 100 and N-substituted piperidin-4-ols 101 in the presence of triphenylphosphine and DEAD or DIAD in THF at room temperature to afford methyl ester intermediate 102. Hydrolysis of the intermediate methyl ester 102 using lithium hydroxide in aqueous THF at ambient temperature afforded the carboxylic acid 103. Carboxylic acid intermediate 103 was subjected to an amide coupling with 4-(2-chlorophenyl)thiazol-2-amine 56 using HATU and DIPEA in DMF at room temperature to afford amide intermediate 104. Removal of the N-Boc protecting group of intermediate 104a was achieved by using a 4 M solution of HCl in dioxane affording piperidine 105 as a hydrochloride salt.

The synthesis of a non-commercial boronic ester 109 is summarised in Scheme 23.

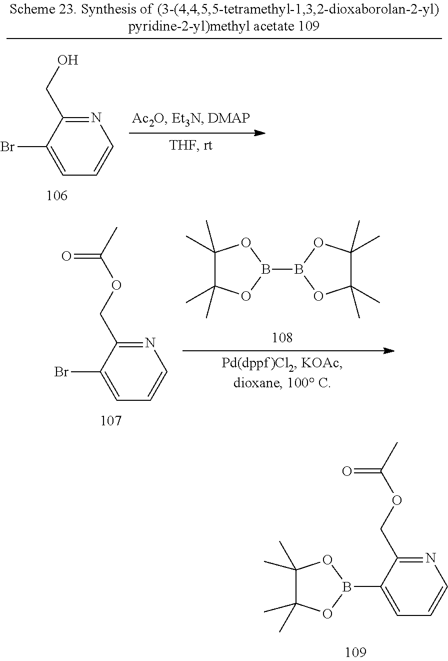

(3-Bromopyridin-2-yl)methanol 106 was protected as an acetate ester 107, using acetic anhydride in the presence of triethylamine and DMAP in DCM at room temperature. Acetate intermediate 107 was reacted with bis(pinacolato)diboron 108, using [1,1′-bis(diphenylphosphino)ferrocene]dichloropalladium (II) as the catalyst in the presence of potassium acetate in dioxane at elevated temperature to afford the heteroaryl boronate reagent 109.

The general synthesis scheme towards RHS-modified analogues of Compound 471 (compound 114) (see Table 1 for structures) is outlined in Scheme 24.

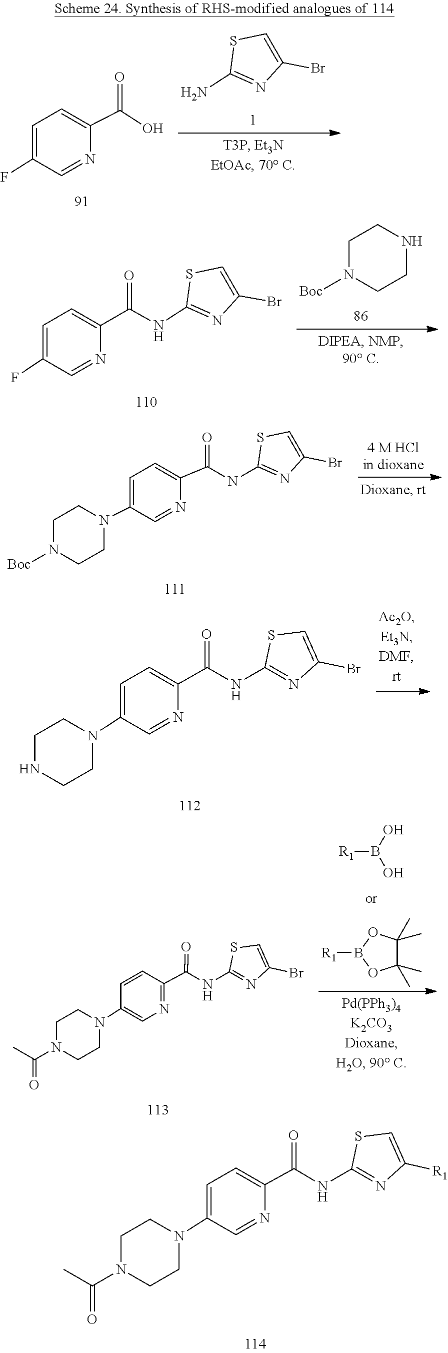

Amide intermediate 110 was synthesized by the reaction of commercially available 4-bromothiazol-2-amine 1 and 5-fluoropicolinic acid 91, in the presence of T3P in ethyl acetate at 70° C. Subsequently, intermediate 111 was generated by a nucleophilic aromatic substitution reaction of amide intermediate 110 with tert-butyl piperazine-1-carboxylate 86 in NMP and DIPEA at 90° C. Removal of the N-Boc protecting group was achieved by treating intermediate 111 with a 4 M solution of HCl in dioxane affording piperazine 112 as a hydrochloride salt. Acetylation of the piperazine 112 using acetic anhydride and triethylamine in DMF at room temperature afforded the N-acetyl piperazine intermediate 113. The final step of the synthesis using intermediate 113 was a Suzuki coupling to deliver the RHS-modified analogues 114. A variety of different aryl boronic acids or pinacol esters 4 were used, using tetrakis(triphenylphosphine)palladium (0) as the catalyst in the presence of potassium carbonate, in a mixture of dioxane and water at elevated temperature.

The synthesis of the amido imidazole analogue 118 (Compound 404) is shown in Scheme 25.

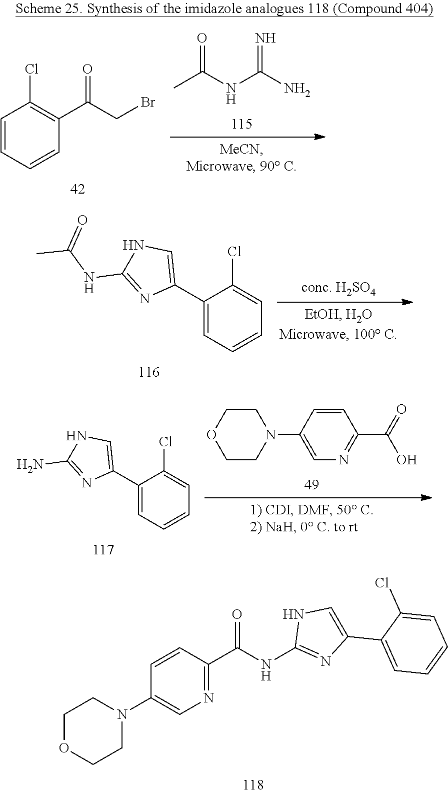

Cyclization via a condensation reaction between 2-bromo-1-(2-chlorophenyl)ethan-1-one 42 and N-carbamimidoylacetamide 115 at 90° C. by microwave heating afforded the acetamido imidazole intermediate 116. Removal of the N-acetyl protecting group was achieved by treatment of the acetamido imidazole 116 with concentrated sulfuric acid in aqueous ethanol by microwave heating at 100° C. The resulting amino imidazole intermediate 117 then underwent coupling with carboxylic acid 49 in the final step, using a similar protocol described in Scheme 22 to afford the amido imidazole analogue 118 (Compound 404).

The three-step synthesis of a non-commercial 4-substituted-2-amine thiazole 122 is summarised in Scheme 26.

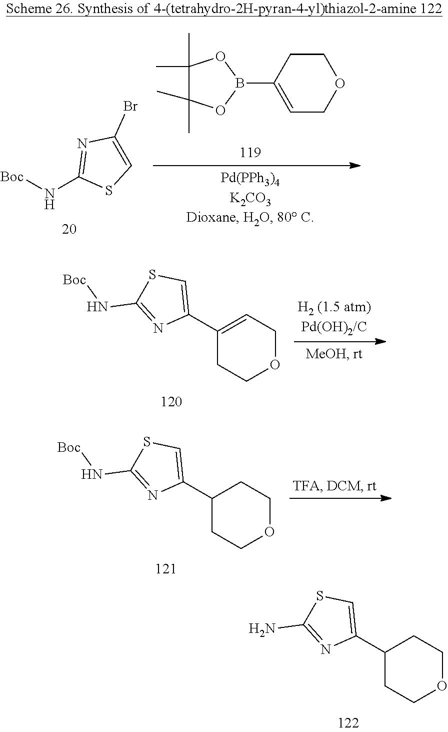

The synthesis started with a Suzuki coupling reaction between tert-butyl (4-bromothiazol-2-yl)carbamate 20 and 2-(3,6-dihydro-2H-pyran-4-yl)-4,4,5,5-tetramethyl-1,3,2-dioxaborolane 119, using tetrakis(triphenylphosphine)palladium (0) as a catalyst with potassium carbonate in aqueous dioxane at 80° C. to afford intermediate 120. In the second step, the double bond of intermediate 120 was reduced by hydrogenation, using Pearlman's catalyst in methanol at room temperature to deliver the tetrahydro-2H-pyran-4-yl intermediate 121. Removal of the N-Boc protecting group was achieved by treating intermediate 121 with trifluoroacetic acid in DCM at room temperature to generate 4-(tetrahydro-2H-pyran-4-yl)thiazol-2-amine 122.

The two-step synthesis of another non-commercial 4-substituted-2-amine thiazole 125 is summarised in Scheme 27.

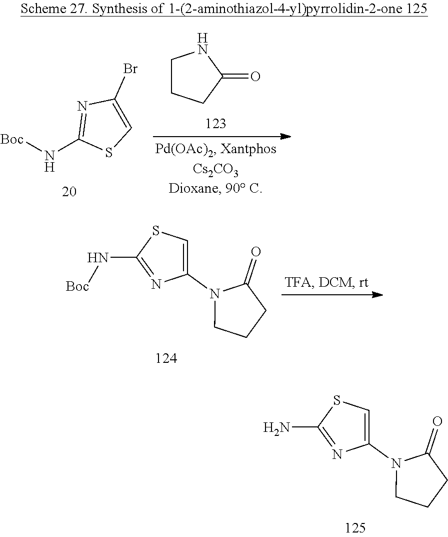

The synthesis of 1-(2-aminothiazol-4-yl)pyrrolidin-2-one 125 started with a Buchwald C—N coupling reaction between tert-butyl (4-bromothiazol-2-yl)carbamate 20 and pyrrolidin-2-one 123 to give N-Boc protected intermediate 124. The final step involved removal of the N-Boc protecting group by treating intermediate 124 with trifluoroacetic acid in DCM at room temperature, which gave 1-(2-aminothiazol-4-yl)pyrrolidin-2-one 125.

The non-commercial 4-substituted-2-amine thiazole 127 was synthesized using the Hantzsch thiazole cyclization as summarised in Scheme 28.

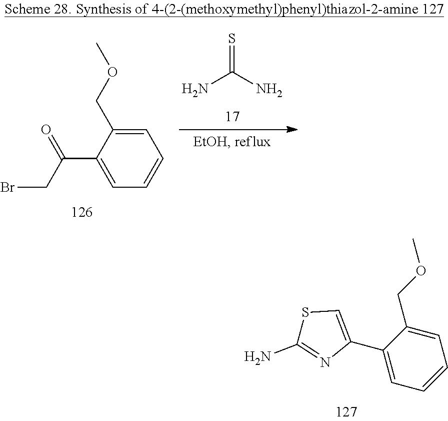

Commercial available 2-bromo-1-(2-(methoxymethyl)phenyl)ethan-1-one 126 was heated at reflux in ethanol with thiourea 17 to afford the 2-amino thiazole 127.

The two-step synthesis of three non-commercial 4-morpholino-2-substituted-benzoic acids 130a, 130b and 130c is summarised below in Scheme 29.

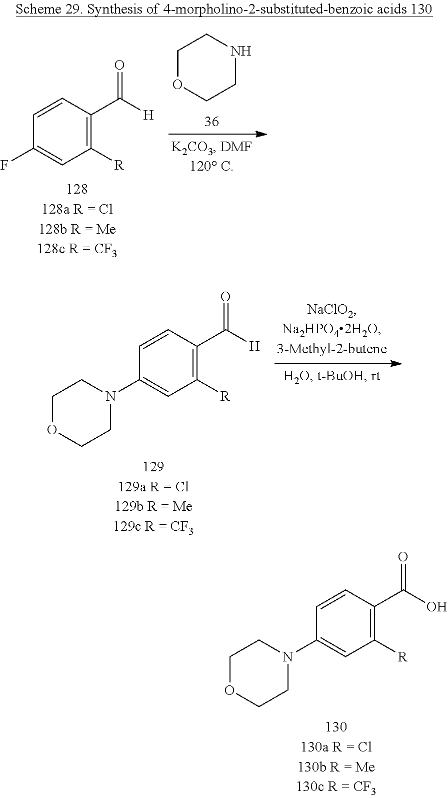

The first step involved nucleophilic aromatic substitution of commercial 4-fluorobenzaldehydes 128 with morpholine 36, in the presence of potassium carbonate in DMF at 120° C. which gave the 2-substituted-4-morpholinobenzaldehyde intermediates 129. Finally, the aldehyde moiety of intermediate 129 was oxidized to the carboxylic acids 130 by the Pinnick oxidation reaction.

The two-step synthesis of non-commercial 2-methoxy-4-morpholinobenzoic acid 130d is summarised in Scheme 30.

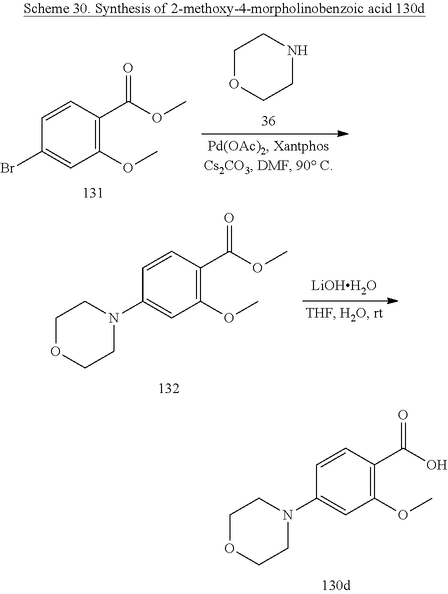

The first step involved a Buchwald C—N coupling reaction between methyl 4-bromo-2-methoxybenzoate 131 and morpholine 36 to give the methyl ester intermediate 132. The methyl ester was subsequently hydrolyzed using lithium hydroxide in aqueous THF at room temperature to deliver the non-commercial reagent, 2-methoxy-4-morpholinobenzoic acid 130d.

The synthesis of three non-commercially available carboxylic acids 138a, 138b and 138c is summarised in Scheme 31.

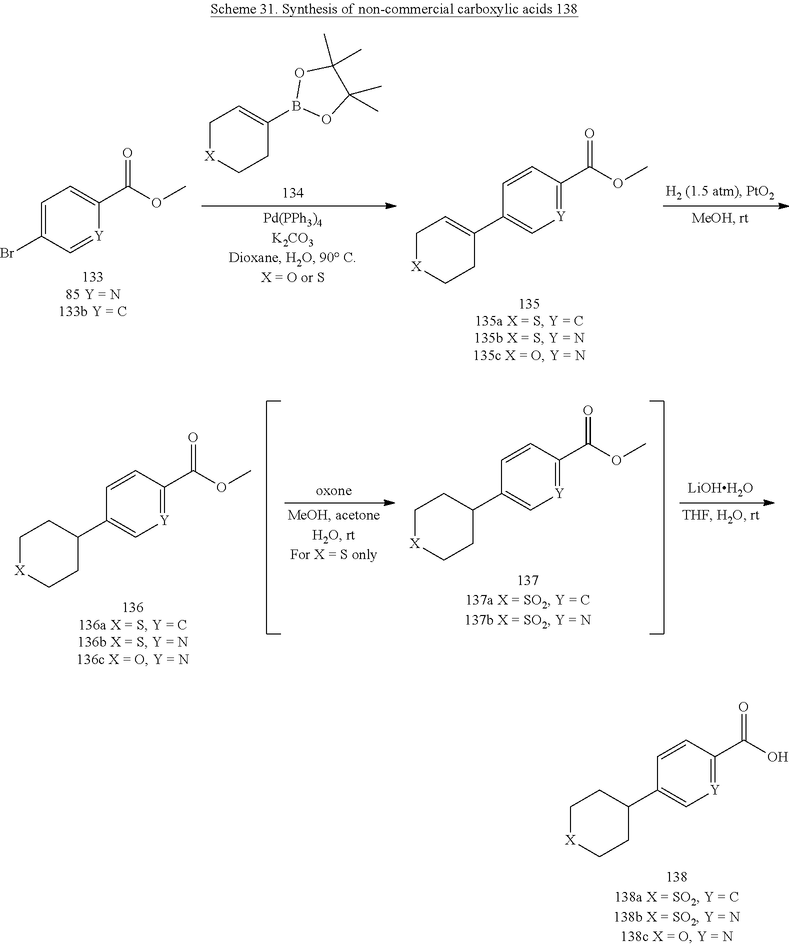

The first step involved a Suzuki coupling reaction between commercial aryl bromide methyl esters 133 and boronic esters 134 (X=O or S) using tetrakis(triphenylphosphine)palladium (0) as a catalyst in the presence of potassium carbonate and a mixture of dioxane and water to afford intermediates 135. In the second step, the double bond of 135 was reduced by hydrogenation, using platinum(IV) oxide (Adams catalyst) in methanol to give methyl ester intermediates 136. For intermediates 136 where X was sulfur, the tetrahydro-2H-thiopyran moiety was oxidized to the tetrahydro-2H-thiopyran 1,1-dioxide, using oxone as an oxidant in a mixed solvent of methanol, acetone and water at room temperature to generate the methyl ester intermediates 137. The final step of the synthesis involved hydrolysis of the methyl ester moiety of intermediates 136 or 137, using lithum hydroxide in aqueous THF to deliver the carboxylic acid intermediates 138. The overall synthetic sequence was 4 steps for carboxylic acids 138a and 138b, while it was just 3 steps for the carboxylic acid 138c.

The two-step synthesis of some other non-commercially available 5-(aminoalkyl)picolinic acids 141 is summarised in Scheme 32.

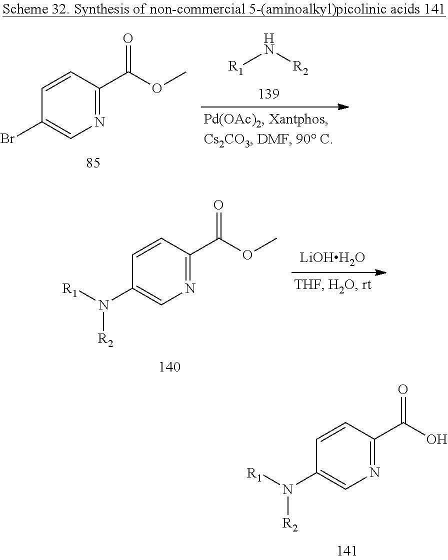

The synthetic sequence in Scheme 32 was similar to the first two steps described in Scheme 19. The first step involved a Buchwald C—N coupling reaction between methyl 5-bromopicolinate 85 and a variety of different amines 139 to generate the methyl ester intermediates 140. Hydrolysis of the intermediate methyl ester using lithium hydroxide in aqueous THF at ambient temperature afforded the carboxylic acid intermediates 141.

A seven-step synthesis of compound 150 (Compound 454) is summarised in Scheme 33.

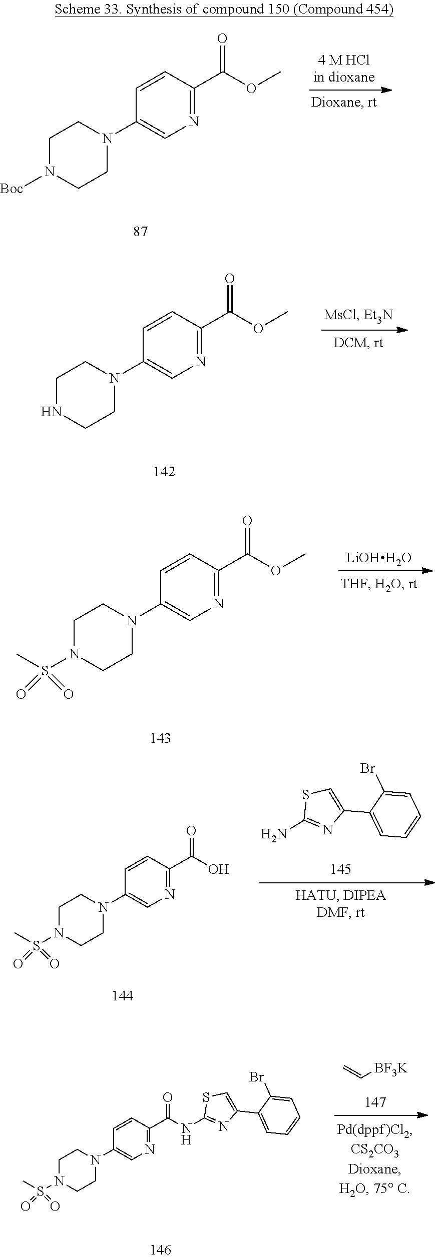

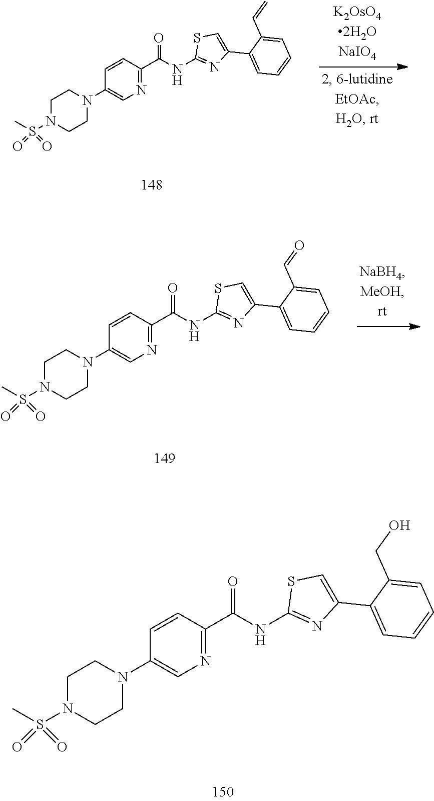 [SMILES: C=Cc1ccccc1-c1csc(NC(=O)c2ccc(N3CCN(S(C)(=O)=O)CC3)cn2)n1 ; CS(=O)(=O)N1CCN(c2ccc(C(=O)Nc3nc(-c4ccccc4C=O)cs3)nc2)CC1 ; CS(=O)(=O)N1CCN(c2ccc(C(=O)Nc3nc(-c4ccccc4CO)cs3)nc2)CC1]

The carboxylic acid 144 was prepared in 3 steps from commercially available tert-butyl 4-(6-(methoxycarbonyl)pyridin-3-yl)piperazine-1-carboxylate 87. Removal of the N-Boc protecting group of starting material 87 with a 4 M solution of HCl in dioxane afforded intermediate piperazine 142. Sulfonylation of the piperazine intermediate 142 by reaction with methanesulfonyl chloride in the presence of triethylamine in DCM gave the sulfonamide intermediate 143. Subsequently, the methyl ester of intermediate 143 was hydrolyzed using lithium hydroxide in aqueous THF at room temperature to give the carboxylic acid 144.

Carboxylic acid intermediate 144 was subjected to an amide coupling in step four with 4-(2-bromophenyl)thiazol-2-amine 145, using HATU and DIPEA in DMF at room temperature to afford amide intermediate 146. Palladium-catalyzed cross-coupling of the 2-bromophenyl moiety of intermediate 146, using commercially available potassium vinyltrifluoroborate 147 in the presence of [1,1-bis(diphenylphosphino)ferrocene]dichloropalladium(II) and cesium carbonate in aqueous dioxane at elevated temperature afforded the 2-vinylphenyl intermediate 148. Oxidative cleavage of the 2-vinylphenyl intermediate 148 facilitated synthesis of the aldehyde intermediate 149. Oxidatve cleavage was performed using potassium osmate(VI) dihydrate and sodium periodate, in the presence of 2,6-lutidine in a mixture of ethyl acetate and water at ambient temperature. In the final step, reduction of the aldehyde group of intermediate 149 using sodum borohydride in methanol at room temperature delivered the final primary alcohol compound 150.

The synthesis of non-commercially available 3-(4-(methylsulfonyl)piperazin-1-yl)bicyclo[1.1.1]pentane-1-carboxylic acid 157 is summarised in Scheme 34.

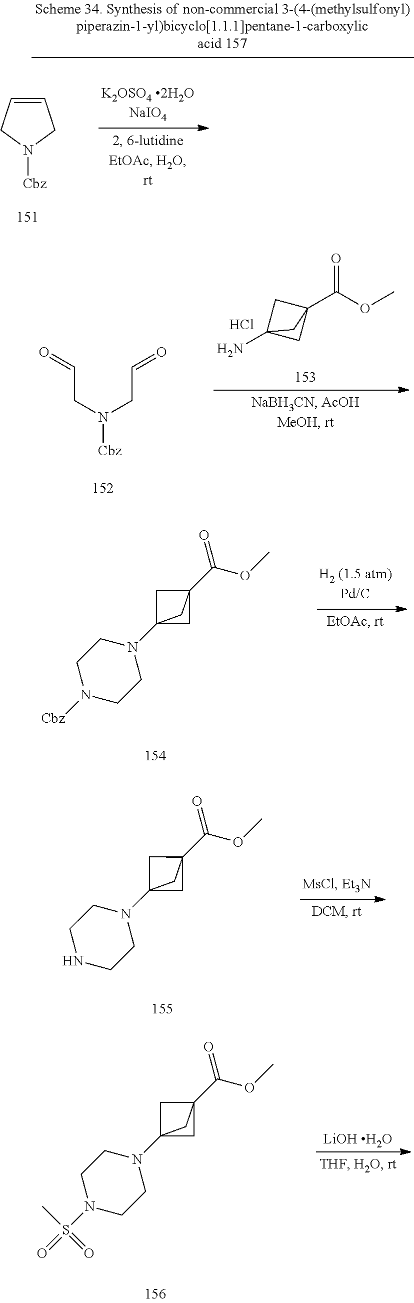

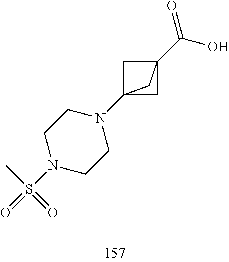 [SMILES: CS(=O)(=O)N1CCN(C23CC(C(=O)O)(C2)C3)CC1]

Oxidative cleavage of commercially available benzyl 2,5-dihydro-1H-pyrrole-1-carboxylate 151, using similar conditions to those described in Scheme 33 afforded the bis-aldehyde intermediate 152. Reductive amination of the bis-aldehyde intermediate 152 with methyl 3-aminobicyclo[1.1.1]pentane-1-carboxylate hydrochloride 153, in the presence of sodium cyanoborohydride and acetic acid in methanol gave the N-Cbz protected piperazine intermediate 154. Removal of the N-Cbz protecting group by palladium-catalyzed hydrogenation in ethyl acetate at ambient temperature generated the piperazine intermediate 155 as a free base. Sulfonylation of the piperazine intermediate 155 by reaction with methanesulfonyl chloride in the presence of triethylamine in DCM gave the sulfonamide intermediate 156. In the final step, the methyl ester of intermediate 156 was hydrolyzed using lithium hydroxide in aqueous THF at room temperature to give the desired carboxylic acid 157.

The synthesis of non-commercially available 3-morpholinobicyclo[1.1.1]pentane-1-carboxylic acid 160 and (1R,3R)-3-morpholinocyclobutane-1-carboxylic acid 163 is summarised in Scheme 35.

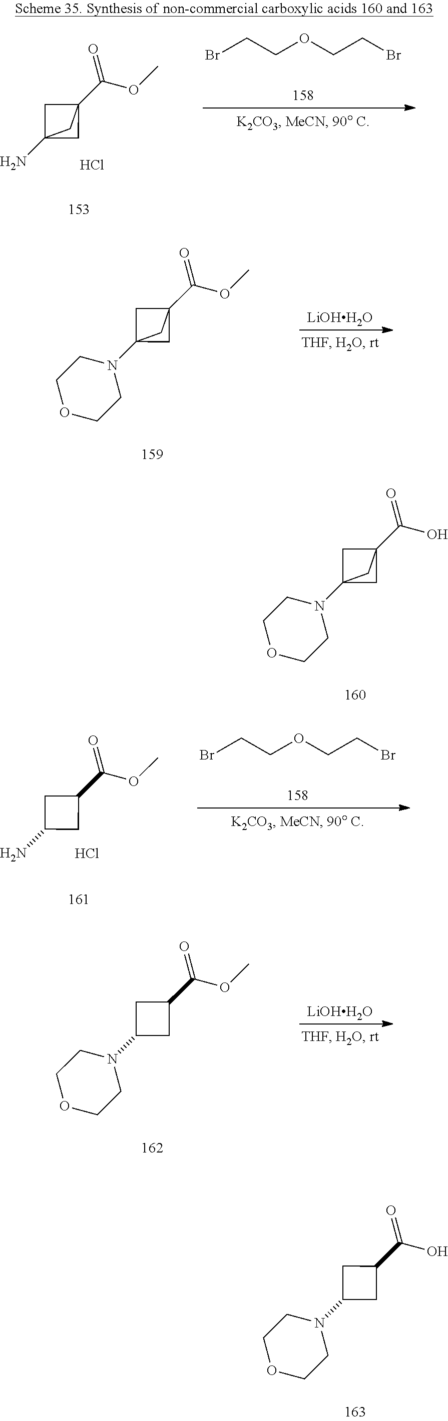

The amino moiety of starting materials 153 and 161 was subjected to a N-di-alkylation cyclisation step, using 1-bromo-2-(2-bromoethoxy)ethane 158 to generate the morpholine ring in intermediates 159 and 162, respectively. The N-di-alkylation cyclisation reaction was performed using potassium carbonate as the base in acetonitrile at 90° C. In the final step, the methyl esters of intermediates 159 and 162 were hydrolyzed using lithium hydroxide in aqueous THF at room temperature to give the desired carboxylic acids 160 and 163 respectively.

The four-step synthesis of compound 168 (Compound 455) is summarised in Scheme 36.

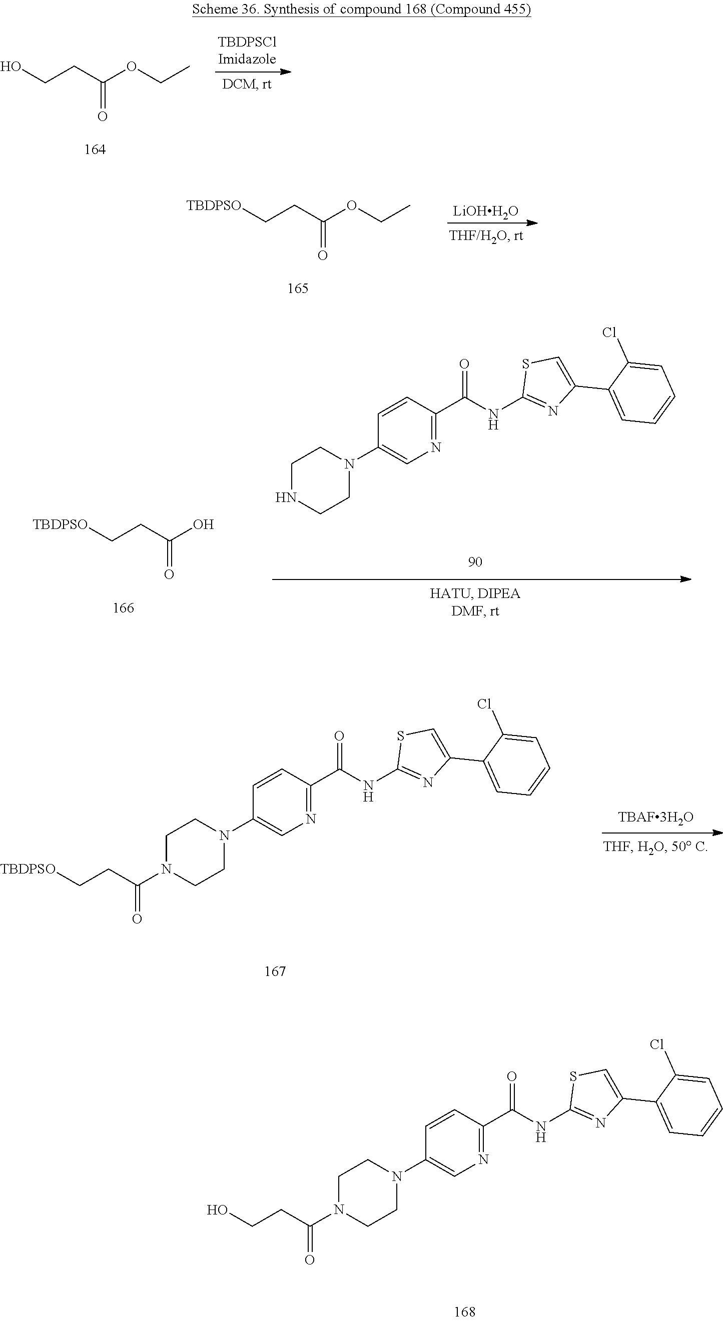

Carboxylic acid 166 was synthesized in two steps from commercially available ethyl 3-hydroxypropanoate 164. The first step involved protection of the primary alcohol of starting material 164 with a tert-butyldiphenylsilyl (TBDPS) protecting group, using TBDPSCl in the presence of imidazole in DCM at room temperature to give ethyl ester intermediate 165. In the second step, the ethyl ester of intermediate 156 was hydrolyzed, using lithium hydroxide in aqueous THF at room temperature to give the carboxylic acid intermediate 166. Carboxylic acid intermediate 166 was subjected to an amide coupling with piperazine intermediate 90, using HATU and DIPEA in DMF at room temperature to afford piperazine amide intermediate 167. In the final step, removal of the O-TBDPS protecting group of intermediate 167, using TBAF in aqueous THF at elevated temperature delivered the final compound 168 (Compound 455).

The synthesis of compound 171 (Compound 460) is summarised in Scheme 37.

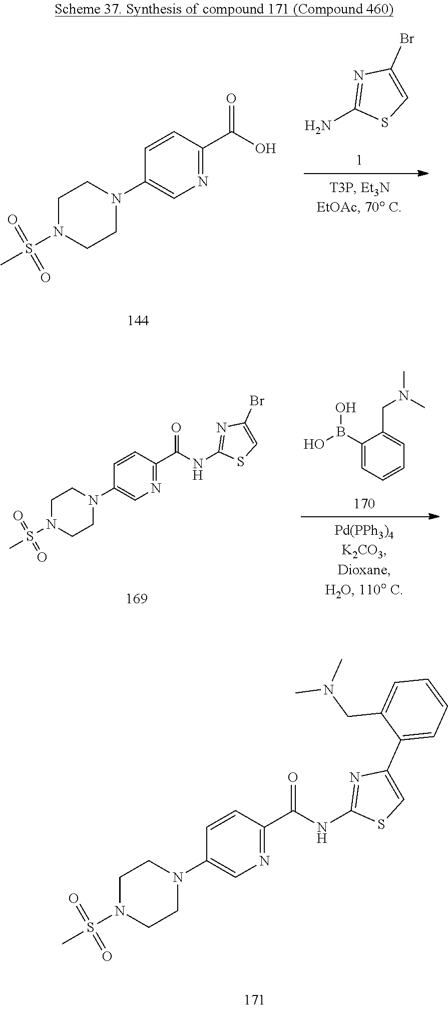 [SMILES: CS(=O)(=O)N1CCN(c2ccc(C(=O)O)nc2)CC1 ; Nc1nc(Br)cs1 ; CS(=O)(=O)N1CCN(c2ccc(C(=O)Nc3nc(Br)cs3)nc2)CC1 ; CN(C)Cc1ccccc1B(O)O ; CN(C)Cc1ccccc1-c1csc(NC(=O)c2ccc(N3CCN(S(C)(=O)=O)CC3)cn2)n1]

The first step of the synthesis involved an amide coupling reaction of 4-bromothiazol-2-amine 1 with 5-(4-(methylsulfonyl)piperazin-1-yl)picolinic acid 144, in the presence of propylphosphonic anhydride (T3P) in ethyl acetate at 70° C. affording amide intermediate 169. In the final step, a palladium-catalyzed Suzuki coupling reaction between 4-bromothiazole amide intermediate 169 and 2-[(dimethylamino)methyl]phenylboronic acid 170 was employed to synthesize the final compound 171 (Compound 460). Reaction conditions for the Suzuki coupling reaction, used tetrakis(triphenylphosphine)palladium (0) as a catalyst with potassium carbonate as base in aqueous dioxane at 110° C.

The synthesis of compound 176 (Compound 468) is summarised in Scheme 38.

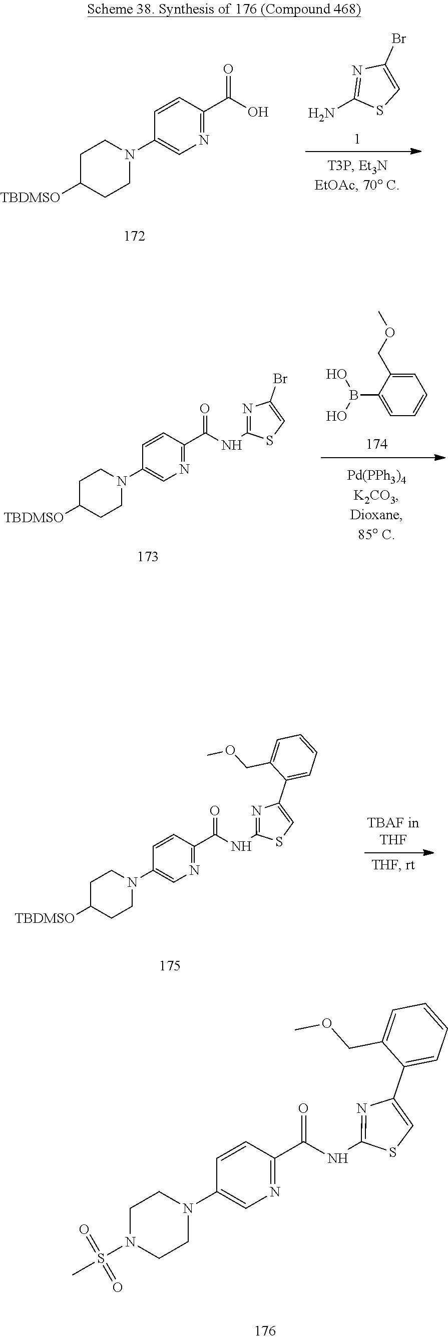 [SMILES: CC(C)(C)[Si](C)(C)OC1CCN(c2ccc(C(=O)O)nc2)CC1 ; Nc1nc(Br)cs1 ; CC(C)(C)[Si](C)(C)OC1CCN(c2ccc(C(=O)Nc3nc(Br)cs3)nc2)CC1 ; COCc1ccccc1B(O)O ; COCc1ccccc1-c1csc(NC(=O)c2ccc(N3CCC(O[Si](C)(C)C(C)(C)C)CC3)cn2)n1 ; COCc1ccccc1-c1csc(NC(=O)c2ccc(N3CCN(S(C)(=O)=O)CC3)cn2)n1]

Carboxylic acid 172 was synthesized using the chemistry described in Scheme 32 (172 was one of the examples of carboxylic acid 141 in Scheme 32). The first step of the synthesis involved an amide coupling reaction of 4-bromothiazol-2-amine 1 with 5-(4-((tert-butyldimethylsilyl)oxy)piperidin-1-yl)picolinic acid 172 in the presence of propylphosphonic anhydride (T3P) in ethyl acetate at 70° C. affording amide intermediate 173. In step two, a palladium-catalyzed Suzuki coupling reaction between 4-bromothiazole amide intermediate 173 and 2-(methoxymethyl)phenylboronic acid 174 was used to afford the 0-TBDMS protected intermediate 175. In the final step, removal of the 0-TBDMS protecting group of intermediate 175 was achieved by treatment with TBAF in THF at room temperature to give the final compound 176 (Compound 468).

The two-step synthesis of three final compounds 179, 182 and 185 (see Table 1 for structures) is outlined in Scheme 39.

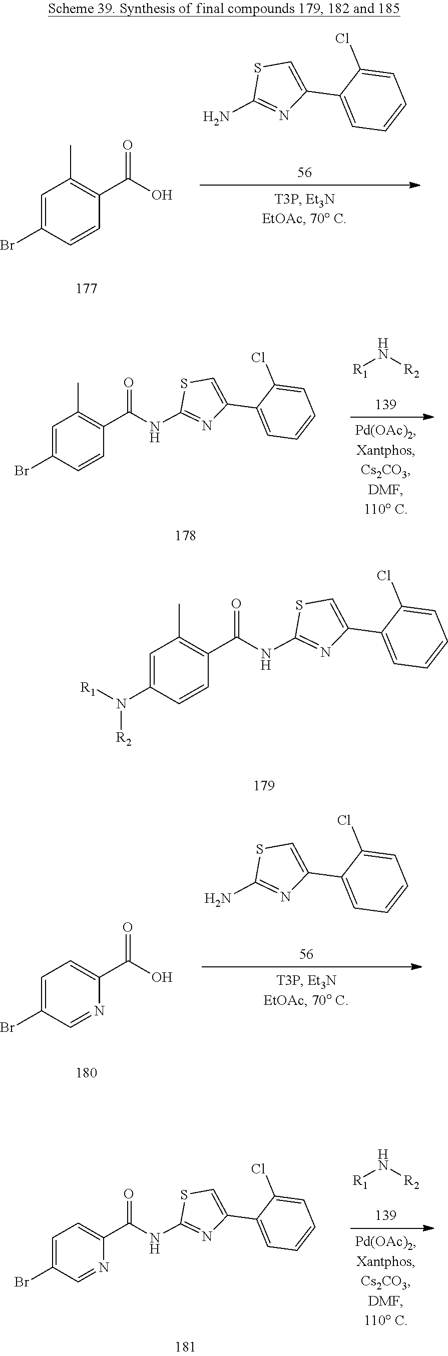

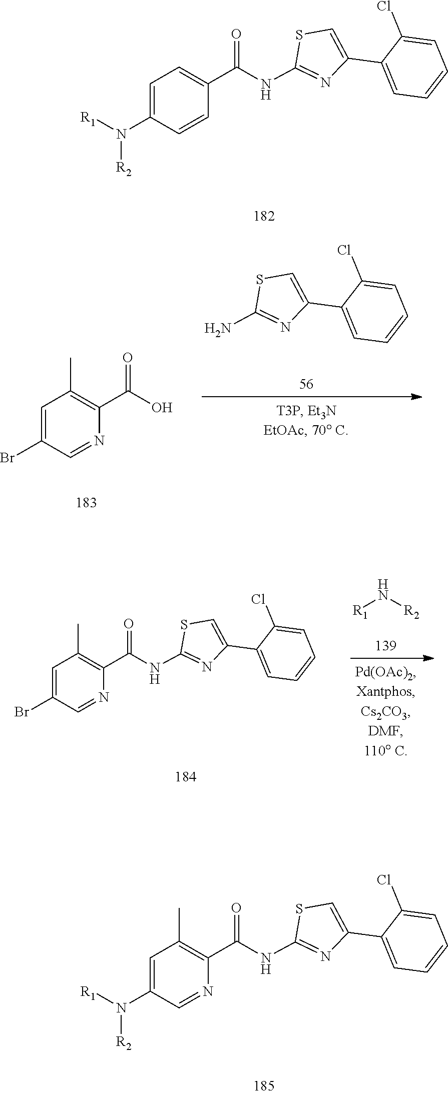

The first step of the synthesis involved an amide coupling reaction of 4-(2-chlorophenyl)thiazol-2-amine 56 with the corresponding acids 177, 180 and 183 in the presence of propylphosphonic anhydride (T3P) at 70° C. affording amide intermediates 178, 181 and 184 respectively. The final step involved a palladium-catalyzed Buchwald C—N coupling reaction between the aryl/heteroaryl bromide moiety of the amide intermediates 178, 181 and 184 with a variety of secondary amines 139 to afford the final compounds 179, 182 and 185 respectively.

The synthesis of compounds 186 is outlined in Scheme 40.

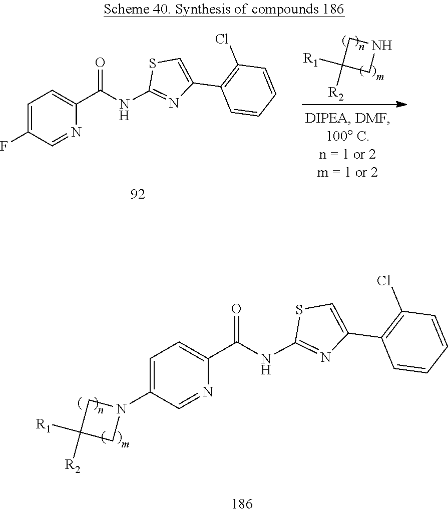

Aryl nucleophilic substitution (S_{N}Ar) reaction between N-(4-(2-chlorophenyl)thiazol-2-yl)-5-fluoropicolinamide 92 and a variety of secondary cyclic amines afforded final compounds 186. The reactions were performed in DMF in the presence of DIPEA as the base at elevated temperature.

The synthesis of final compounds 188a and 189 is outlined in Scheme 41.

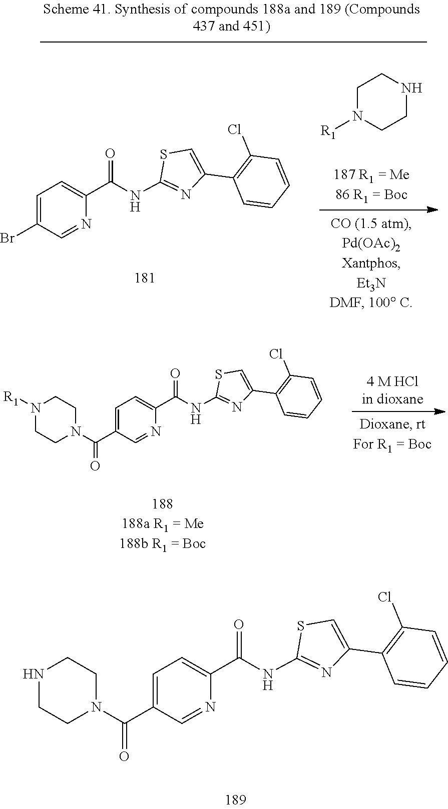

The first step of the synthesis involved a palladium-catalyzed direct amidation reaction using 5-bromo-N-(4-(2-chlorophenyl)thiazol-2-yl)picolinamide 181, carbon monoxide (for CO insertion) and either 1-methylpiperazine 187 or tert-butyl piperazine-1-carboxylate 86 as the secondary amine, which gave either final compound 188a or intermediate 188b respectively. Removal of the N-Boc protecting group was achieved by treating intermediate 188b with a 4 M solution of HCl in dioxane to afford the final piperazine compound 189.

The related syntheses of compounds 193 and 196 (Compounds 463 and 462) are summarised in Scheme 42.

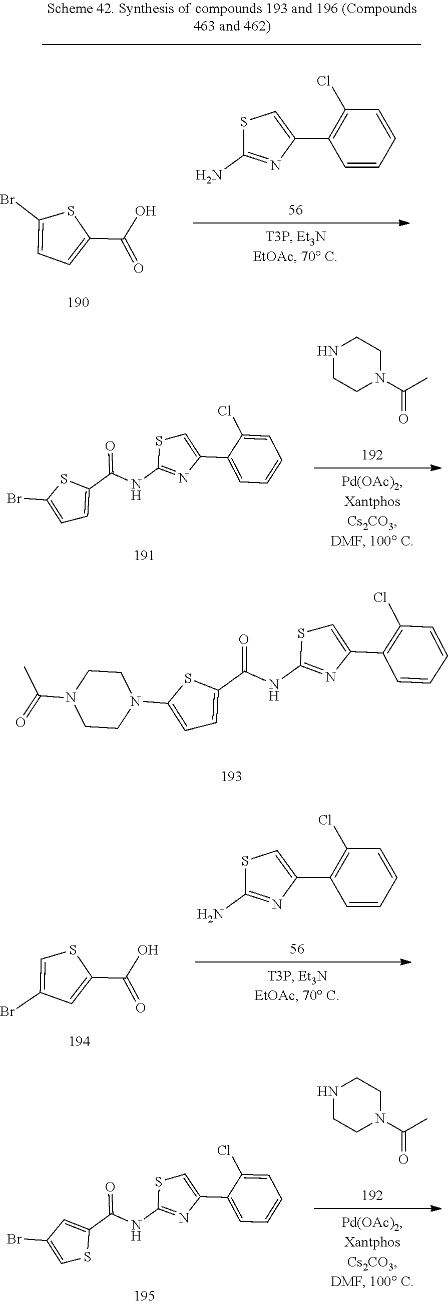

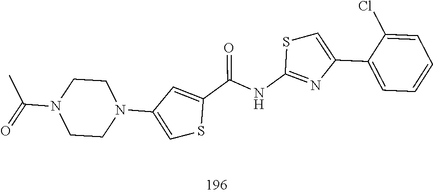 [SMILES: CC(=O)N1CCN(c2csc(C(=O)Nc3nc(-c4ccccc4Cl)cs3)c2)CC1]

The first step of the synthesis involved an amide coupling reaction between 4-(2-chlorophenyl)thiazol-2-amine 56 and carboxylic acids 190 and 194 in the presence of propylphosphonic anhydride (T3P) at 70° C. affording amide intermediates 191 and 195 respectively. The final step involved a palladium-catalyzed Buchwald C—N coupling reaction between the heteroaryl bromide moiety of the amide intermediates 191 and 195 with 1-acetylpiperazine 192 to afford the final compounds 193 and 196 respectively.

The syntheses of compound 200, 201 and 204 (Compounds 435, 434 and 421) are summarised in Scheme 43.

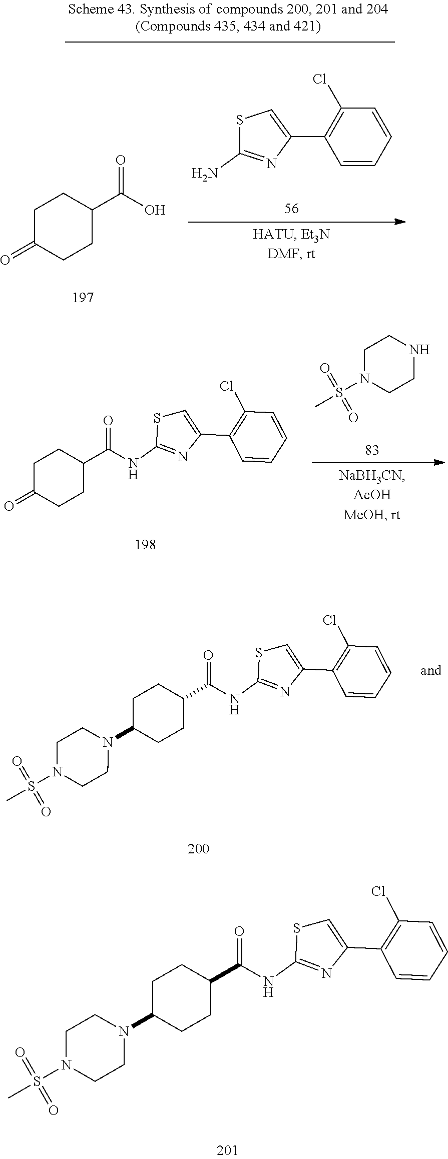

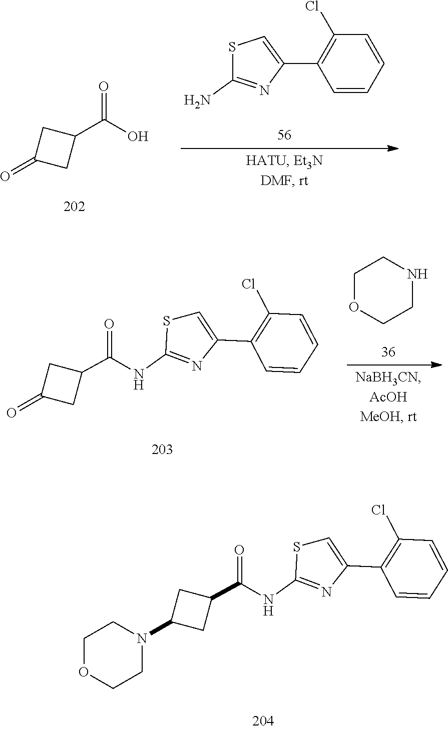 [SMILES: O=C1CC(C(=O)O)C1 ; Nc1nc(-c2ccccc2Cl)cs1 ; O=C1CC(C(=O)Nc2nc(-c3ccccc3Cl)cs2)C1 ; C1COCCN1 ; O=C(Nc1nc(-c2ccccc2Cl)cs1)[C@H]1C[C@@H](N2CCOCC2)C1]

The first step of the synthesis involved an amide coupling reaction of 4-(2-chlorophenyl)thiazol-2-amine 56 with the carboxylic acids 197 and 202 in the presence of HATU and triethylamine in DMF at room temperature affording amide intermediates 198 and 203 respectively. The final step was a reductive alkylation reaction of the ketone functional group of intermediates 198 or 203 with 1-(methylsulfonyl)piperazine 199 and morpholine 36 respectively. The reactions were performed in methanol using sodium cyanoborohydride and acetic acid to afford the final compounds 200, 201 and 204.

The syntheses of amides 206, 208, 210 and 212 are summarised in Scheme 44.

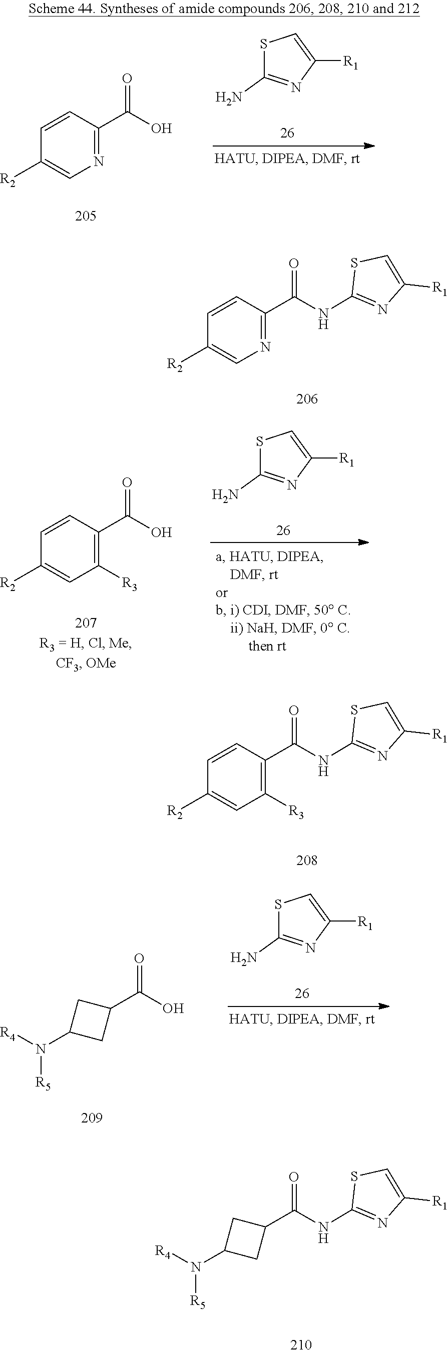

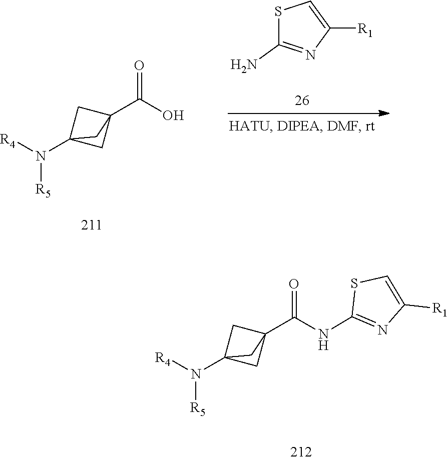

Carboxylic acids 205, 207 (different R₃ substituents), 209 and 211 were used as the starting materials with thiazole amines 26 via an amide formation to synthesize the final amide compounds 206, 208, 210 and 212 respectively. In general, reactions were performed in DMF using HATU and DIPEA at room temperature. Alternative reaction conditions can be employed in the case of carboxylic acids 207, which involve a two-step protocol to prepare the final compound amides 208. The carboxylic acids 207 were activated first using CDI in DMF at 50° C. and then treated in a second step with the thiazole amine intermediates 26, which have been subjected to deprotonation with sodium hydride as a strong base in DMF.

The synthesis of the amide compounds 216 is summarised in Scheme 45.

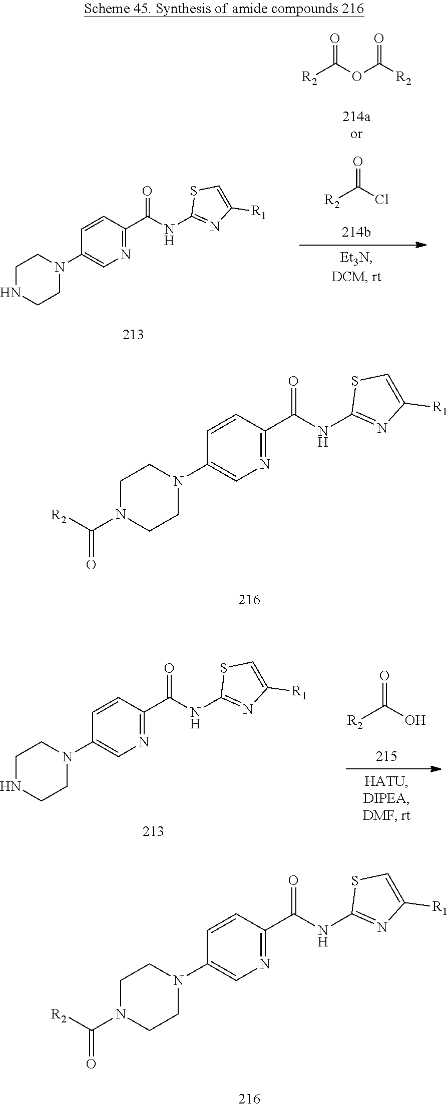

Amide compounds 216 were synthesized by reaction between the NH of the piperazine group of 213 with either acid anhydrides 214a or various acid chlorides 214b. Reactions were performed in DCM in the presence of triethylamine as the base at room temperature. Alternatively, amide formation can be carried out using carboxylic acids 215 in the presence of HATU and DIPEA in DMF at room temperature.

The synthesis of sulfonamide compounds 217 is summarised in Scheme 46.

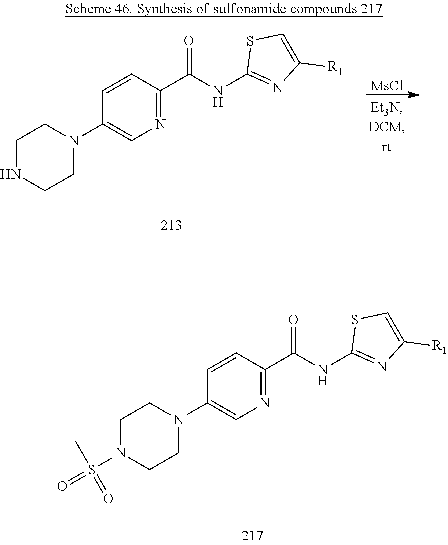

Sulfonamide compounds 217 were synthesized by sulfonylation of the NH of the piperazine group of 213 with methansulfonyl chloride, using triethylamine in DCM at room temperature.

The synthesis of the N-alkylated compounds 219 is summarised in Scheme 47.

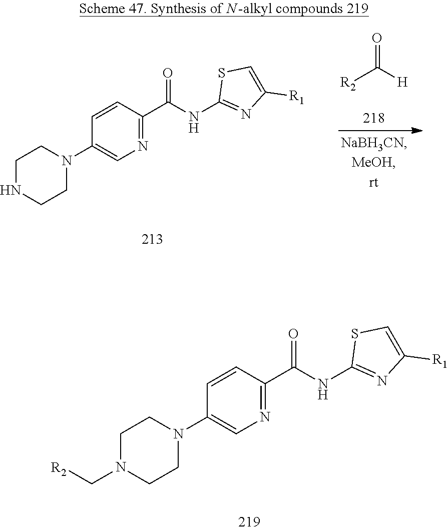

N-alkyl compounds 219 were synthesized by reductive alkylation of the NH of the piperazine group of 213 with various aldehydes 218. Reactions were performed using sodium cyanoborohydride in methanol at room temperature.

The synthesis of N-alkyl compounds 221 is summarised in Scheme 48.

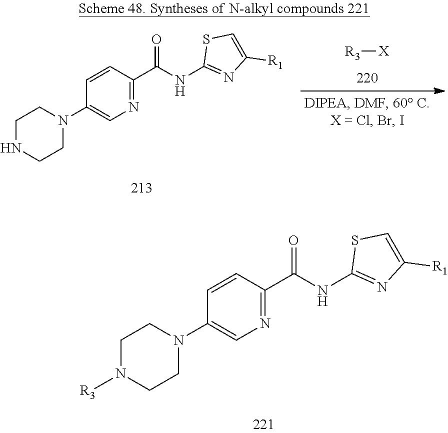

N-alkyl compounds 221 were synthesized by direct alkylation of the NH of the piperazine group of 213 with various alkyl halides 220. Reactions were performed using DIPEA as a base in DMFat elevated temperatures.

### Detailed Synthesis of Intermediates of Compounds of the Invention

### Synthesis of N-(4-bromothiazol-2-yl)-4-methoxybenzamide

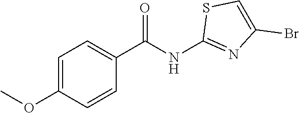 [SMILES: COc1ccc(C(=O)Nc2nc(Br)cs2)cc1]

To a solution of 4-bromothiazol-2-amine (1 g, 5.59 mmol) and 4-methoxybenzoic acid (1.28 g, 8.38 mmol) in anhydrous DCM (10 mL) was added triethylamine (4.7 mL, 33.5 mmol) followed by a solution of T3P (50% in ethyl acetate, 10 mL, 33.5 mmol). The reaction mixture was heated at 40° C. for 18 hours. After cooling to room temperature, the mixture was partitioned between DCM (30 ml) and water (30 mL). The layers were separated, and the organic phase was washed with brine (50 mL). The organic layer was dried (MgSO₄), filtered and concentrated under reduced pressure. The residue was purified by column chromatography on silica gel (0-25% ethyl acetate in cyclohexane) to afford N-(4-bromothiazol-2-yl)-4-methoxybenzamide as a pale yellow solid.

### Yield 1.32 g (75%). ¹H NMR (400 MHz, DMSO) δ 12.82 (br s, 1H), 8.12 (d, J=9.0 Hz, 2H), 7.39 (s, 1H), 7.21 (d, J=9.0 Hz, 2H), 3.94 (s, 3H).

### Synthesis of N-(4-bromothiazol-2-yl)-6-methylnicotinamide

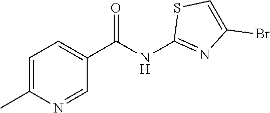 [SMILES: Cc1ccc(C(=O)Nc2nc(Br)cs2)cn1]

To a solution of 4-bromothiazol-2-amine (1 g, 5.59 mmol) and 6-methylnicotinic acid (1.15 g, 8.38 mmol) in anhydrous DCM (10 mL) was added triethylamine (4.7 mL, 33.5 mmol) followed by a solution of T3P (50% in ethyl acetate, 10 mL, 33.5 mmol). The reaction mixture was heated at 45° C. for 18 hours. After cooling to room temperature, the mixture was partitioned between DCM (30 ml) and water (30 mL). The layers were separated, and the organic phase was washed with brine (50 mL). The organic layer was dried (MgSO₄), filtered and concentrated under reduced pressure. The residue was purified by column chromatography on silica gel (0-50% ethyl acetate in cyclohexane) to afford N-(4-bromothiazol-2-yl)-6-methylnicotinamide as a pale yellow solid.

### Yield 898 mg (54%). ¹H NMR (400 MHz, DMSO) δ 13.09 (s, 1H), 9.11 (d, J=2.0 Hz, 1H), 8.32 (dd, J=2.0, 8.0 Hz, 1H), 7.46 (d, J=8.0 Hz, 1H), 7.41 (s, 1H), 2.58 (s, 3H).

### Synthesis of tert-butyl (4-(2,4-dichlorophenyl)thiazol-2-yl)carbamate

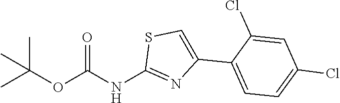 [SMILES: CC(C)(C)OC(=O)Nc1nc(-c2ccc(Cl)cc2Cl)cs1]

To a mixture of tert-butyl 4-bromothiazol-2-ylcarbamate (1.5 g, 5.37 mmol) and (2,4-dichlorophenyl)boronic acid (2.05 g, 10.75 mmol) in 1,4-dioxane (25 mL) was added [1,1′-bis(diphenylphosphino)ferrocene] dichloropalladium(II), complex with dichloromethane (439 mg, 0.537 mmol), and a solution of sodium carbonate (1.71 mg, 16.12 mmol) in water (1 mL). The reaction mixture was heated at 85° C. for 18 hours. After cooling to room temperature, the mixture was diluted with EtOAc (50 mL) and was filtered through a plug of Celite. The filtrate was collected, washed with brine (50 mL), dried (MgSO4), filtered and evaporated. The residue was purified by column chromatography on silica gel (0-50% ethyl acetate in cyclohexane) to afford tert-butyl (4-(2,4-dichlorophenyl)thiazol-2-yl)carbamate as an off-white foam.

### Yield 1.46 g (79%). ¹H NMR (400 MHz, DMSO) δ 11.63 (br s, 1H), 7.87 (d, J=8.5 Hz, 1H), 7.71 (d, J=2.1 Hz, 1H), 7.64 (s, 1H), 7.53 (dd, J=2.2, 8.5 Hz, 1H), 1.51 (s, 9H).

### Synthesis of 4-(2,4-dichlorophenyl)thiazol-2-amine

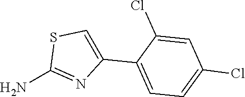 [SMILES: Nc1nc(-c2ccc(Cl)cc2Cl)cs1]

To a solution of tert-butyl (4-(2,4-dichlorophenyl)thiazol-2-yl)carbamate (1.46 g, 4.23 mmol) in 1,4-dioxane (8 ml) was added a 4 M solution of HCl in 1,4-dioxane (4 mL, 16 mmol). The reaction mixture was stirred at room temperature for 18 hours and then heated at 50° C. for 3 days. After cooling to room temperature, the mixture was partitioned between ethyl acetate (15 ml) and water (20 mL). The aqueous layer was extracted with ethyl acetate (2×15 mL) and the combined organic extracts dried (MgSO₄), filtered and concentrated to afford 4-(2,4-dichlorophenyl)thiazol-2-amine as a pale yellow solid. Yield 1 g (96%). ¹H NMR (400 MHz, DMSO) δ 7.89 (d, J=8.6 Hz, 1H), 7.66 (s, 1H), 7.48 (d, J=8.6 Hz, 1H), 7.11 (m, 3H).

### Synthesis of 1-(2-bromophenyl)azetidine

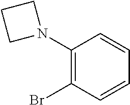 [SMILES: Brc1ccccc1N1CCC1]

To a degassed mixture of 1-bromo-2-iodobenzene (0.14 mL, 1.06 mmol), azetidine (0.086 mL, 1.27 mmol) and sodium tert-butoxide (357 mg, 3.71 mmol) in THF (4 mL) was added tris(dibenzylideneacetone)dipalladium(0) (97 mg, 0.106 mmol) and rac-BINAP (330 mg, 0.53 mmol). The reaction mixture was stirred at 50° C. for 18 hours. After cooling to room temperature, the mixture was diluted with ethyl acetate (20 mL), filtered through Celite and the filtrate was evaporated. The orange residue was purified by column chromatography on silica gel (0-10% ethyl acetate in cyclohexane) to afford 1-(2-bromophenyl)azetidine as a colourless oily solid.

Yield 168 mg (75%). ¹H NMR (400 MHz, CDCl₃) δ 7.40 (dd, J=1.5, 8.0 Hz, 1H), 7.17 (dd, J=6.9, 8.4 Hz, 1H), 6.66 (dt, J=1.5, 6.9 Hz, 1H), 6.54 (dd, J=1.5, 8.4 Hz, 1H), 4.06 (dd, J=7.3, 7.3 Hz, 4H), 2.30-2.22 (m, 2H).

### Synthesis of tert-butyl (4-(2-chlorophenyl)thiazol-2-yl)carbamate

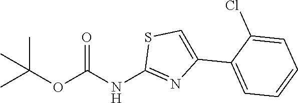 [SMILES: CC(C)(C)OC(=O)Nc1nc(-c2ccccc2Cl)cs1]

To a degassed (nitrogen) mixture of tert-butyl 4-bromothiazol-2-ylcarbamate (1 g, 3.58 mmol) and (2-chlorophenyl)boronic acid (1.12 g, 7.16 mmol) in 1,4-dioxane (15 mL) was added [1,1′-bis(diphenylphosphino)ferrocene] dichloropalladium(II), complex with dichloromethane (293 mg, 0.358 mmol), and a solution of sodium carbonate (1.139 g, 10.7 mmol) in water (1 mL). The reaction mixture was sparged with nitrogen for 5 minutes and heated at 90° C. for 18 hours. After cooling to room temperature, the solvents were evaporated and the residue was purified by column chromatography on silica gel (0-25% ethyl acetate in cyclohexane) to afford tert-butyl (4-(2-chlorophenyl)thiazol-2-yl)carbamate as a yellow solid.

Yield 1.2 g (quantitative). ¹H NMR (400 MHz, DMSO) δ 11.60 (s, 1H), 7.83 (dd, J=1.9, 7.7 Hz, 1H), 7.57 (s, 1H), 7.55 (dd, J=1.5, 7.8 Hz, 1H), 7.42 (dd, J=1.5, 7.8 Hz, 1H), 7.38 (dd, J=1.9, 7.7 Hz, 1H), 1.44 (s, 9H).

### Synthesis of 4-(2-chlorophenyl)thiazol-2-amine

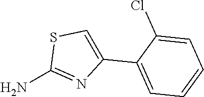 [SMILES: Nc1nc(-c2ccccc2Cl)cs1]

tert-Butyl (4-(2-chlorophenyl)thiazol-2-yl)carbamate (1.1 g, 3.58 mmol) was dissolved in a 4 M solution of HCl in 1,4-dioxane (5.4 mL, 19.3 mmol) and the reaction mixture was stirred at room temperature for 18 hours. The mixture was partitioned between ethyl acetate (15 mL) and water (20 mL). The layers were separated, and aqueous layer was extracted with ethyl acetate (2×15 mL) and the combined organic extracts dried (MgSO₄), filtered and concentrated to afford 4-(2-chlorophenyl)thiazol-2-amine as a yellow solid.

### Yield 646 mg (86%). ¹H NMR (400 MHz, DMSO) δ 7.85 (dd, J=1.8, 7.8 Hz, 1H), 7.50 (dd, J=1.2, 7.9 Hz, 1H), 7.41 (dd, J=1.2, 7.9 Hz, 1H), 7.36 (dd, J=1.8, 7.8 Hz, 1H), 7.07 (br s, 2H), 7.05 (s, 1H).

### Synthesis of tert-butyl 4-(4-((4-(2-chlorophenyl)thiazol-2-yl)carbamoyl)phenyl)-piperazine-1-carboxylate (Method 1)

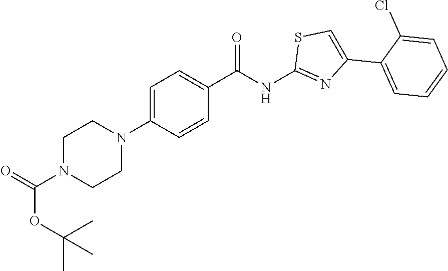 [SMILES: CC(C)(C)OC(=O)N1CCN(c2ccc(C(=O)Nc3nc(-c4ccccc4Cl)cs3)cc2)CC1]

Compound tert-butyl 4-(4-((4-(2-chlorophenyl)thiazol-2-yl)carbamoyl)phenyl)piperazine-1-carboxylate was prepared from 4-(4-(tert-butoxycarbonyl)piperazin-1-yl)benzoic acid following a similar procedure to that described for the synthesis of N-[4-(2-chlorophenyl)thiazol-2-yl]-6-methyl-pyridine-3-carboxamide, except that it was purified by column chromatography on silica gel (0-100% ethyl acetate in cyclohexane), and was isolated as a pale yellow solid.

Yield 320 mg (68%). ¹H NMR (400 MHz, DMSO) δ 12.46 (br s, 1H), 8.07 (d, J=9.0 Hz, 2H), 7.92 (dd, J=1.5, 8.0 Hz, 1H), 7.63 (s, 1H), 7.58 (dd, J=1.5, 8.0 Hz, 1H), 7.45 (dd, J=1.5, 7.6 Hz, 1H), 7.41 (dd, J=1.5, 7.6 Hz, 1H), 7.04 (d, J=9.0 Hz, 2H), 3.51-3.44 (m, 4H), 3.39-3.35 (m, 4H), 1.44 (s, 9H).

### Synthesis of N-(4-bromothiazol-2-yl)-2-methoxypyrimidine-5-carboxamide

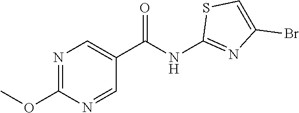 [SMILES: COc1ncc(C(=O)Nc2nc(Br)cs2)cn1]

To a solution of 4-bromothiazol-2-amine (726 mg, 4.06 mmol) and 2-methoxypyrimidine-5-carboxylic acid (750 mg, 4.87 mmol) in anhydrous DCM (4 ml) was added triethylamine (3.4 mL, 24.3 mmol) followed by a solution of T3P (50% in ethyl acetate, 7.2 mL, 24.3 mmol). The reaction mixture was heated at 45° C. for 18 hours. After cooling to room temperature, the mixture was partitioned between ethyl acetate (30 mL) and water (30 mL). The layers were separated, and the aqueous layer was extracted with ethyl acetate (2×20 mL). The combined organic extracts were dried (MgSO₄), filtered and evaporated. The residue was purified by column chromatography on silica gel (0-25% ethyl acetate in cyclohexane) to afford N-(4-bromothiazol-2-yl)-2-methoxypyrimidine-5-carboxamide as a pale yellow solid.

### Yield 978 mg (76%). ¹H NMR (400 MHz, DMSO) δ 13.13 (s, 1H), 9.21 (s, 2H), 7.42 (s, 1H), 4.03 (s, 3H).

### Synthesis of N-(4-bromothiazol-2-yl)-4-morpholinobenzamide

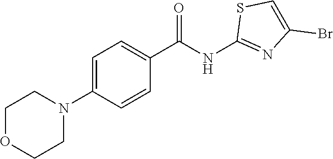 [SMILES: O=C(Nc1nc(Br)cs1)c1ccc(N2CCOCC2)cc1]

To a solution of 4-bromothiazol-2-amine (1 g, 5.59 mmol) and 4-morpholinobenzoic acid (1.74 g, 8.38 mmol) in anhydrous DCM (10 mL) was added triethylamine (4.7 mL, 33.5 mmol) followed by a solution of T3P (50% in ethyl acetate, 10 mL, 33.5 mmol). The reaction mixture was heated at 45° C. for 18 hours. After cooling to room temperature, the mixture was partitioned between DCM (30 mL) and water (30 mL). The layers were separated, and the aqueous layer was extracted with DCM (2×20 mL). The combined organic extracts were dried (MgSO₄), filtered and evaporated. The residue was purified by column chromatography on silica gel (0-25% ethyl acetate in cyclohexane) to afford N-(4-bromothiazol-2-yl)-4-morpholinobenzamide as a pale yellow solid.

### Yield 1.19 g (58%). ¹H NMR (400 MHz, DMSO) δ 12.65 (s, 1H), 8.06 (d, J=9.1 Hz, 2H), 7.36 (s, 1H), 7.08 (d, J=9.1 Hz, 2H), 3.81-3.77 (m, 4H), 3.35-3.30 (m, 4H).

### Synthesis of phenyl (4-(2-chlorophenyl)thiazol-2-yl)carbamate

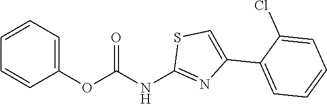 [SMILES: O=C(Nc1nc(-c2ccccc2Cl)cs1)Oc1ccccc1]

To a solution of 4-(2-chlorophenyl)thiazol-2-amine (150 mg, 0.712 mmol) in pyridine (3 mL) was added phenyl chloroformate (0.11 mL, 0.854 mmol). The reaction mixture was stirred at room temperature for 18 hours. The reaction mixture was partitioned between ethyl acetate (20 mL) and brine (20 mL). The layers were separated and the aqueous layer was further extracted with ethyl acetate (2×20 mL). The organic layers were combined, dried (MgSO₄), filtered and evaporated. The residue was purified by column chromatography on silica gel (0-25% ethyl acetate in cyclohexane) to afford phenyl (4-(2-chlorophenyl)thiazol-2-yl)carbamate as an off-white solid.

Yield 262 mg (quantitative). ¹H NMR (400 MHz, DMSO) δ 12.49 (s, 1H), 7.87 (dd, J=1.6, 7.8 Hz, 1H), 7.67 (s, 1H), 7.58 (dd, J=1.6, 7.8 Hz, 1H), 7.49 (d, J=7.6 Hz, 2H), 7.40 (d, J=7.6 Hz, 2H), 7.35 (s, 1H), 7.33-7.28 (m, 2H).

### Synthesis of 4-(pyridin-2-yl)thiazol-2-amine

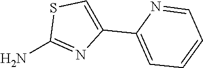 [SMILES: Nc1nc(-c2ccccn2)cs1]

To a solution of 2-bromo-1-(pyridin-2-yl)ethan-1-one (300 mg, 1.50 mmol) in ethanol (5 mL) was added thiourea (171 mg, 2.25 mmol). The reaction was stirred at reflux for 4 hours and cooled to room temperature. The solvent was evaporated and the residue partitioned between DCM (20 mL) and a saturated solution of NaHCO₃ (20 mL). The layers were separated and the aqueous layer further extracted with DCM (2×20 mL). The organic layers were combined, dried (MgSO₄) and filtered. The solvent was evaporated to afford 4-(pyridin-2-yl)thiazol-2-amine as a brown solid. The material was taken forward crude without purification.

### Yield 229 mg (86%). ¹H NMR (400 MHz, DMSO) δ 8.58 (dt, J=1.3, 4.7 Hz, 1H), 7.89-7.83 (m, 2H), 7.32-7.28 (m, 2H), 7.15 (br s, 2H).

### Synthesis of 4-(2-chlorophenyl)oxazol-2-amine

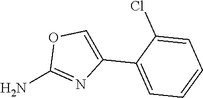 [SMILES: Nc1nc(-c2ccccc2Cl)co1]

To a solution of 2-bromo-1-(2-chlorophenyl)ethan-1-one (530 mg, 2.27 mmol) in anhydrous DMF (3 mL) was added urea (1.36 g, 22.70 mmol). The reaction mixture was heated in a microwave at 130° C. for 30 minutes and cooled to room temperature. The solvent was evaporated and the residue dissolved in DCM (30 mL) and washed with water (30 mL). The layers were separated using a phase separator and the DCM was evaporated to give a residue, which was purified by column chromatography on silica gel (0-50% ethyl acetate in cyclohexane) to afford 4-(2-chlorophenyl)oxazol-2-amine as an off-white solid.

Yield 230 mg (52%). ¹H NMR (400 MHz, CDCl₃) δ 7.98 (dd, J=1.5, 7.9 Hz, 1H), 7.88 (s, 1H), 7.41 (dd, J=1.5, 7.9 Hz, 1H), 7.31 (dt, J=1.5, 7.6 Hz, 1H), 7.20 (dt, J=1.5, 7.6 Hz, 1H), 4.72 (s, 2H).

### Synthesis of methyl 5-morpholinopyrazine-2-carboxylate

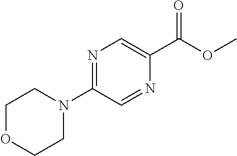 [SMILES: COC(=O)c1cnc(N2CCOCC2)cn1]

To a solution of methyl 5-chloropyrazine-2-carboxylate (250 mg, 1.45 mmol) in 1,4-dioxane was added triethylamine (0.5 mL, 3.62 mmol) and morpholine (0.15 mL, 1.74 mmol). The reaction was heated in a microwave at 100° C. for 30 minutes and cooled to room temperature. The reaction mixture was partitioned between ethyl acetate (30 mL) and water (30 mL), then the layers were separated. The aqueous layer was further extracted with ethyl acetate (2×20 mL) and the organic layers combined. The combined organic layers were dried (MgSO₄), filtered and the solvent removed by evaporation to afford methyl 5-morpholinopyrazine-2-carboxylate as an off-white solid.

Yield 305 mg (94%). ¹H NMR (400 MHz, CDCl₃) δ 8.81 (d, J=1.3 Hz, 1H), 8.13 (d, J=1.3 Hz, 1H), 3.96 (s, 3H), 3.85-3.81 (m, 4H), 3.75-3.71 (m, 4H).

### Synthesis of 5-chloropyrazine-2-carboxylic acid hydrochloride

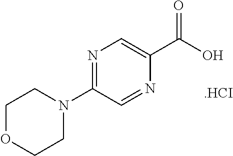

A solution of methyl 5-morpholinopyrazine-2-carboxylate (305 mg, 1.37 mmol) in 6 M hydrochloric acid (10 mL) was heated at reflux for 3 hours and cooled to room temperature. The solvent was evaporated to give an off-white solid, which was azeotroped with acetonitrile (3×50 mL) to afford 5-chloropyrazine-2-carboxylic acid hydrochloride as an off-white powder.

Yield 350 mg (quantitative). ¹H NMR (400 MHz, DMSO) δ 8.67 (d, J=1.3 Hz, 1H), 8.37 (d, J=1.3 Hz, 1H), 3.72 (s, 8H). Acid and HCl protons obscured by water peak.

### Synthesis of ethyl 6-morpholinopyridazine-3-carboxylate

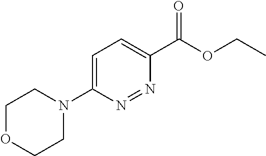 [SMILES: CCOC(=O)c1ccc(N2CCOCC2)nn1]

To a solution of ethyl 6-chloropyridazine-3-carboxylate (250 mg, 1.34 mmol) in 1,4-dioxane (2 mL) was added morpholine (0.14 mL, 1.61 mmol) and triethylamine (0.47 ml, 1.61 mmol). The reaction was heated in a microwave at 100° C. for 30 minutes and cooled to room temperature. The reaction was partitioned between ethyl acetate (20 ml.) and water (20 mL), then the layers were separated. The aqueous layer was further extracted with ethyl acetate (2×20 ml) and the organic layers combined, dried (MgSO₄) and filtered. The solvent was removed by evaporation to afford ethyl 6-morpholinopyridazine-3-carboxylate as an off-white solid.

Yield 292 mg (91%). ¹H NMR (400 MHz, CDCl₃) δ 7.92 (d, J=9.5 Hz, 1H), 6.86 (d, J=9.5 Hz, 1H), 4.47 (q, J=7.1 Hz, 2H), 3.86-3.84 (m, 4H), 3.79-3.75 (m, 4H), 1.44 (t, J=7.1 Hz, 3H).

### Synthesis of 6-morpholinopyridazine-3-carboxylic acid hydrochloride

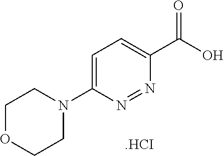

A solution of ethyl 6-morpholinopyridazine-3-carboxylate (292 mg, 1.31 mmol) in 6 M hydrochloric acid (10 mL) was heated at reflux for 3 hours and cooled to room temperature. The solvent was removed by evaporation to give an off white solid, which was azeotroped from acetonitrile (3×30 mL) to give 6-morpholinopyridazine-3-carboxylic acid hydrochloride as an off-white powder.

Yield 318 mg (quantitative). ¹H NMR (400 MHz, DMSO) δ 7.92 (d, J=9.7 Hz, 1H), 7.40 (d, J=9.7 Hz, 1H), 3.78-3.70 (m, 8H). Acid and HCl protons obscured by water peak.

### Synthesis of tert-butyl (4-(2-methylpyridin-3-yl)thiazol-2-yl)carbamate

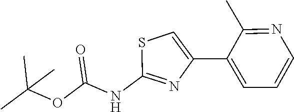 [SMILES: Cc1ncccc1-c1csc(NC(=O)OC(C)(C)C)n1]

To a degassed solution of tert-butyl (4-bromothiazol-2-yl)carbamate (1.50 g, 5.37 mmol) in 1,4-1,4-dioxane (20 mL) and water (5 mL) was added at room temperature sodium carbonate (2.28 g, 21.49 mmol), 2-methylpyridine-3-boronic acid (1.10 g, 8.06 mmol) and [1,1′-bis(diphenylphosphino)-ferrocene]dichloropalladium(II), complex with dichloromethane (439 mg, 0.537 mmol). The reaction was heated at 100° C. for 24 hours and then cooled to room temperature. Ethyl acetate (40 mL) was added and the layers were separated. The organic layer was dried (MgSO₄), filtered and concentrated by evaporation to give a residue, which was purified by column chromatography on silica gel (0-80% ethyl acetate in cyclohexane) to afford tert-butyl (4-(2-methylpyridin-3-yl)thiazol-2-yl)carbamate as an orange solid.

### Yield 682 mg (43%). ¹H NMR (400 MHz, DMSO) δ 11.62 (s, 1H), 8.48 (dd, J=1.6, 4.7 Hz, 1H), 7.96 (dd, J=1.6, 7.6 Hz, 1H), 7.40 (s, 1H), 7.34 (dd, J=4.7, 7.6 Hz, 1H), 2.66 (s, 3H), 1.56 (s, 9H).

### Synthesis of 4-(2-methylpyridin-3-yl)thiazol-2-amine

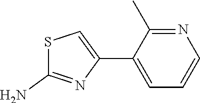

To a solution of tert-butyl (4-(2-methylpyridin-3-yl)thiazol-2-yl)carbamate (682 mg, 2.34 mmol) in DCM (10 mL) was added 4 M hydrogen chloride in 1,4-dioxane (7 mL). The reaction was stirred at room temperature for 24 hours and then at 40° C. for 3 hours. The reaction was cooled to room temperature and ethyl acetate (50 mL) was added. The mixture was neutralised with saturated sodium hydrogen carbonate and the layers separated. The aqueous layer was further extracted with ethyl acetate (50 mL), the organic layers combined, dried (MgSO₄) and filtered. The filtrate was concentrated by evaporation to afford 4-(2-methylpyridin-3-yl)thiazol-2-amine as an orange solid.

Yield 474 mg (quantitative). ¹H NMR (400 MHz, DMSO) δ 8.39 (dd, J=1.8, 4.9 Hz, 1H), 7.91 (dd, J=1.8, 7.9 Hz, 1H), 7.26 (dd, J=4.9, 7.9 Hz, 1H), 7.08 (s, 2H), 6.78 (s, 1H), 2.63 (s, 3H).

### Synthesis of tert-butyl 4-(4-((4-(2-chlorophenyl)thiazol-2-yl)carbamoyl)phenyl)piperazine-1-carboxylate (Method 2)

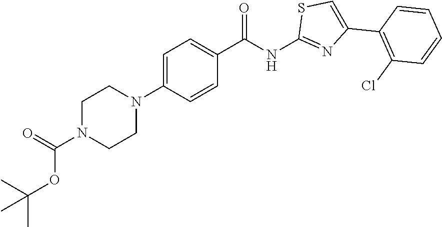 [SMILES: CC(C)(C)OC(=O)N1CCN(c2ccc(C(=O)Nc3nc(-c4ccccc4Cl)cs3)cc2)CC1]

To a solution of 4-(2-chlorophenyl)thiazol-2-amine (1.00 g, 4.75 mmol) in DMF (20 mL) were added 4-[4-(tert-butoxycarbonyl)piperazin-1-yl]benzoic acid (2.18 g, 7.12 mmol), DMAP (0.58 g, 4.75 mmol) and N-(3-dimethylaminopropyl)-N-ethylcarbodiimide hydrochloride (1.09 g, 5.70 mmol) at room temperature under a nitrogen atmosphere. The resulting mixture was stirred at 100° C. for 16 h under nitrogen atmosphere. After cooling down to room temperature, the resulting mixture was quenched with a solution of saturated aqueous NH₄Cl (50 mL) and extracted with ethyl acetate (3×200 mL). The combined organic layers were washed with brine (3×50 mL) and dried over anhydrous Na₂SO₄. After filtration, the filtrate was concentrated under reduced pressure. The residue was purified by silica gel column chromatography, eluted with 1%-40% ethyl acetate in petroleum ether to afford tert-butyl 4-(4-((4-(2-chlorophenyl)thiazol-2-yl)carbamoyl)phenyl)piperazine-1-carboxylate as an off-white solid:

Yield 1.70 g (72%). ¹H NMR (300 MHz, CDCl₃) δ 10.08 (s, 1H), 7.85 (d, J=8.9 Hz, 2H), 7.80 (dd, J=1.9, 7.6, Hz, 1H), 7.49-7.40 (m, 2H), 7.37-7.20 (m, 2H), 6.90 (d, J=8.9 Hz, 2H), 3.61 (t, J=5.3 Hz, 4H), 3.35 (t, J=5.3 Hz, 4H), 1.51 (s, 9H).

¹H NMR (400 MHz, DMSO) δ 12.47 (s, 1H), 8.04 (d, J=8.9 Hz, 2H), 7.91 (dd, J=1.9, 7.7 Hz, 1H), 7.63 (s, 1H), 7.57 (dd, J=1.5, 7.8 Hz 1H), 7.45 (td, J=1.5, 7.5, 1H), 7.39 (td, J=1.8, 7.6 Hz, 1H), 7.04 (d, J=8.9 Hz, 2H), 3.47 (t, J=6.3 Hz, 4H), 3.35 (t, J=6.3 Hz, 4H), 1.43 (s, 9H). m/z: [ESI⁺] 499, 501 (M+H)⁺.

### Synthesis of methyl 4-(3,6-dihydro-2H-thiopyran-4-yl)benzoate

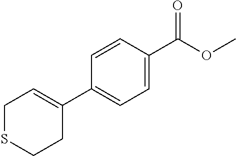 [SMILES: COC(=O)c1ccc(C2=CCSCC2)cc1]

To a mixture of methyl 4-bromobenzoate (2.00 g, 9.30 mmol), potassium carbonate (2.57 g, 18.60 mmol) and 2-(3,6-dihydro-2H-thiopyran-4-yl)-4,4,5,5-tetramethyl-1,3,2-dioxaborolane (3.15 g, 13.93 mmol) in 1,4-dioxane (20 mL) and water (4 ml) was added tetrakis(triphenylphosphine)palladium (0) (3.22 g, 2.79 mmol) at room temperature under a nitrogen atmosphere. The resulting mixture was stirred for 3 h at 85° C. After cooling to room temperature, the resulting mixture was concentrated under reduced pressure. The residue was purified by silica gel column chromatography, eluting with 0-16% ethyl acetate in petroleum ether to afford methyl 4-(3,6-dihydro-2H-thiopyran-4-yl)benzoate as an off-white solid.

Yield 1.00 g (46%). ¹H NMR (400 MHz, CDCl₃) δ 8.01 (d, J=8.4 Hz, 2H), 7.42 (d, J=8.4 Hz, 2H), 6.33-6.30 (m, 1H), 3.94 (s, 3H), 3.43-3.34 (m, 2H), 2.96-2.88 (m, 2H), 2.78-2.70 (m, 2H). No MS signal.

### Synthesis of methyl 4-(tetrahydro-2H-thiopyran-4-yl)benzoate

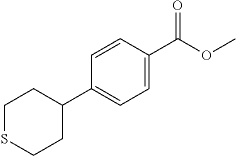 [SMILES: COC(=O)c1ccc(C2CCSCC2)cc1]

To a stirred solution of methyl 4-(3,6-dihydro-2H-thiopyran-4-yl)benzoate (0.40 g, 1.71 mmol) in methanol (8 mL) was added platinum (IV) oxide (0.80 g, 3.52 mmol) at room temperature under a nitrogen atmosphere. The resulting mixture was stirred overnight at room temperature under a hydrogen atmosphere (1.5 atm). The resulting mixture was filtered and the filter cake washed with methanol (3×2 mL). The combined washings and filtrate were concentrated under reduced pressure. The residue was purified by reverse phase flash chromatography with the following conditions: Column: WelFlash TM C18-I, 20-40 μm, 330 g; Eluent A: water (plus 10 mmol/L NH₄HCO₃); Eluent B: acetonitrile; Gradient: 38%-58% B in 25 min; Flow rate: 90 mL/min; Detector: UV 220/254 nm. The fractions containing the desired product were collected and concentrated under reduced pressure to afford methyl 4-(tetrahydro-2H-thiopyran-4-yl)benzoate as an off-white solid.

Yield 0.18 g (45%). ¹H NMR (400 MHz, DMSO) δ 7.90 (d, J=8.4 Hz, 2H), 7.38 (d, J=8.4 Hz, 2H), 3.84 (s, 3H), 2.86-2.73 (m, 2H), 2.69-2.61 (m, 3H), 2.07-2.01 (m, 2H), 1.80-1.65 (m, 2H). No MS signal.

### Synthesis of methyl 4-(1,1-dioxidotetrahydro-2H-thiopyran-4-yl)benzoate

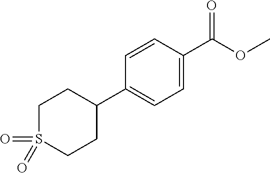 [SMILES: COC(=O)c1ccc(C2CCS(=O)(=O)CC2)cc1]

To a stirred solution of methyl 4-(tetrahydro-2H-thiopyran-4-yl)benzoate (0.18 g, 0.76 mmol) in methanol (16 mL) were added water (3 mL), potassium peroxymonosulfate (Oxone®) (0.48 g, 1.56 mmol) and acetone (4 mL) at 0° C. The resulting mixture was stirred overnight at room temperature. The mixture was concentrated under reduced pressure. The residue was purified by silica gel column chromatography, eluting with 0-50% ethyl acetate in petroleum ether to afford methyl 4-(1,1-dioxidotetrahydro-2H-thiopyran-4-yl)benzoate as an off-white solid.

Yield 160 mg (78%). ¹H NMR (400 MHz, DMSO) δ 7.92 (d, J=8.4 Hz, 2H), 7.43 (d, J=8.4 Hz, 2H), 3.84 (s, 3H), 3.44-3.27 (m, 2H), 3.18-3.08 (m, 2H), 3.08-2.98 (m, 1H), 2.16-2.04 (m, 4H). No MS signal.

### Synthesis of 4-(1,1-dioxidotetrahydro-2H-thiopyran-4-yl)benzoic acid

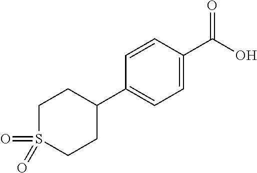 [SMILES: O=C(O)c1ccc(C2CCS(=O)(=O)CC2)cc1]

To a stirred solution of methyl 4-(1,1-dioxidotetrahydro-2H-thiopyran-4-yl)benzoate (160 mg, 0.596 mmol) in water (2 mL) and THF (2 mL) was added lithium hydroxide monohydrate (100 mg, 2.383 mmol) at room temperature. The resulting mixture was stirred overnight at room temperature. The pH value of the solution was adjusted to 5 with a solution of aqueous 2 M HCl and the mixture concentrated under reduced pressure. The residue was purified by reverse phase flash chromatography with the following conditions: Column: WelFlash TM C18-I, 20-40 μm, 330 g; Eluent A: water (plus 10 mmol/L HCOOH); Eluent B: acetonitrile; Gradient: 5%-20% B in 25 min; Flow rate: 80 mL/min; Detector: UV 220/254 nm. The desired fractions were collected and concentrated under reduced pressure to afford 4-(1,1-dioxidotetrahydro-2H-thiopyran-4-yl)benzoic acid as an off-white solid.

Yield 142 mg (94%). ¹H NMR (400 MHz, DMSO) δ 512.85 (br s, 1H), 7.90 (d, J=8.4 Hz, 2H), 7.40 (d, J=8.4 Hz, 2H), 3.38-3.28 (m, 2H), 3.19-3.08 (m, 2H), 3.08-2.96 (m, 1H), 2.16-2.05 (m, 4H). m/z: [ESI⁻] 253 (M−H)⁻.

### Synthesis of methyl 5-(3,6-dihydro-2H-thiopyran-4-yl)picolinate

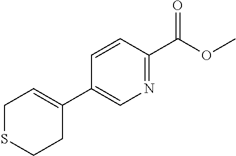 [SMILES: COC(=O)c1ccc(C2=CCSCC2)cn1]

Methyl 5-(3,6-dihydro-2H-thiopyran-4-yl)picolinate was prepared from methyl 5-bromopicolinate (2.40 g, 11.11 mmol) and 2-(3,6-dihydro-2H-thiopyran-4-yl)-4,4,5,5-tetramethyl-1,3,2-dioxaborolane (2.50 g, 11.06 mmol) following a procedure similar to that described for the synthesis of methyl 4-(3,6-dihydro-2H-thiopyran-4-yl)benzoate and was isolated as a yellow solid.

Yield 1.71 g (66%). ¹H NMR (400 MHz, DMSO) δ 8.77 (d, J=2.0 Hz, 1H), 8.02 (d, J=8.0 Hz, 1H), 7.98 (dd, J=2.0, 8.0 Hz, 1H), 6.58-6.48 (m, 1H), 3.88 (s, 3H), 3.33-3.38 (m, 2H), 2.87 (t, J=5.6 Hz, 2H), 2.71-2.65 (m, 2H). m/z: [ESI⁺] 236 (M+H)⁺.

### Synthesis of methyl 5-(tetrahydro-2H-thiopyran-4-yl)picolinate

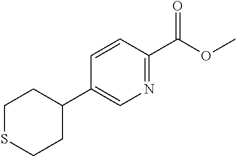 [SMILES: COC(=O)c1ccc(C2CCSCC2)cn1]

Compound methyl 5-(tetrahydro-2H-thiopyran-4-yl)picolinate was prepared from methyl 5-(3,6-dihydro-2H-thiopyran-4-yl)picolinate (1.71 g, 7.27 mmol) following a procedure similar to that described for the synthesis of methyl 4-(tetrahydro-2H-thiopyran-4-yl)benzoate, and was isolated as an off-white solid.

Yield 0.83 g (48%). ¹H NMR (400 MHz, DMSO) δ 8.61 (d, J=2.0 Hz, 1H), 8.00 (d, J=8.0 Hz, 1H), 7.86 (dd, J=2.0, 8.0 Hz, 1H), 3.87 (s, 3H), 2.86-2.72 (m, 3H), 2.72-2.63 (m, 2H), 2.12-2.01 (m, 2H), 1.86-1.70 (m, 2H). m/z: [ESI⁺] 238 (M+H)⁺.

### Synthesis of methyl 5-(1,1-dioxidotetrahydro-2H-thiopyran-4-yl)picolinate

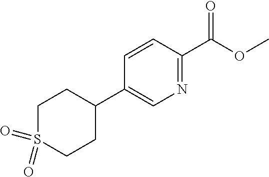 [SMILES: COC(=O)c1ccc(C2CCS(=O)(=O)CC2)cn1]

Compound methyl 5-(1,1-dioxidotetrahydro-2H-thiopyran-4-yl)picolinate was prepared from methyl 5-(tetrahydro-2H-thiopyran-4-yl)picolinate (0.80 g, 3.37 mmol) and potassium peroxymonosulfate (Oxone®) (4.31 g, 14.04 mmol) following a procedure similar to that described for the synthesis of methyl 4-(1,1-dioxidotetrahydro-2H-thiopyran-4-yl)benzoate, and was isolated as an off-white solid.

Yield 0.65 g (72%). ¹H NMR (400 MHz, DMSO) δ 8.66 (d, J=2.0 Hz, 1H), 8.03 (d, J=8.0 Hz, 1H), 7.96 (dd, J=2.0, 8.0 Hz, 1H), 3.88 (s, 3H), 3.39-3.28 (m, 2H), 3.20-3.07 (m, 3H), 2.20-2.10 (m, 4H). m/z: [ESI⁺]270 (M+H)⁺.

### Synthesis of 5-(1,1-dioxidotetrahydro-2H-thiopyran-4-yl)picolinic acid

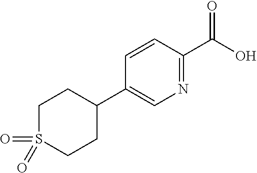 [SMILES: O=C(O)c1ccc(C2CCS(=O)(=O)CC2)cn1]

Compound 5-(1,1-dioxidotetrahydro-2H-thiopyran-4-yl)picolinic acid was prepared from methyl 5-(1,1-dioxidotetrahydro-2H-thiopyran-4-yl)picolinate (0.50 g, 1.86 mmol) and lithium hydroxide monohydrate (0.30 g, 7.15 mmol), following a procedure similar to that described for the synthesis of 4-(1,1-dioxidotetrahydro-2H-thiopyran-4-yl)benzoic acid, and was isolated as an off-white solid.

Yield 0.30 g (63%). ¹H NMR (400 MHz, DMSO) δ 13.10 (br s, 1H), 8.62 (d, J=2.0 Hz, 1H), 8.00 (d, J=8.0 Hz, 1H), 7.89 (dd, J=2.0, 8.0 Hz, 1H), 3.40-3.28 (m, 2H), 3.19-2.99 (m, 3H), 2.21-2.09 (m, 4H). m/z: [ESI⁺]256 (M+H)⁺.

### Synthesis of methyl 5-(3,6-dihydro-2H-pyran-4-yl)picolinate

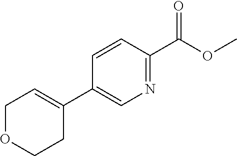 [SMILES: COC(=O)c1ccc(C2=CCOCC2)cn1]

Compound methyl 5-(3,6-dihydro-2H-pyran-4-yl)picolinate was prepared from methyl 5-bromopicolinate (216 mg, 1.00 mmol) and 2-(3,6-dihydro-2H-pyran-4-yl)-4,4,5,5-tetramethyl-1,3,2-dioxaborolane (210 mg, 1.00 mmol) following a procedure similar to that described for the synthesis of methyl 4-(3,6-dihydro-2H-thiopyran-4-yl)benzoate, and was isolated as an off-white solid.

Yield 120 mg (55%). m/z: [ESI⁺] 220 (M+H)⁺.

### Synthesis of methyl 5-(tetrahydro-2H-pyran-4-yl)picolinate

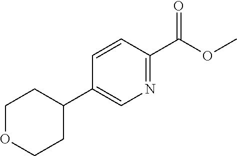 [SMILES: COC(=O)c1ccc(C2CCOCC2)cn1]

Compound methyl 5-(tetrahydro-2H-pyran-4-yl)picolinate was prepared from methyl 5-(3,6-dihydro-2H-pyran-4-yl)picolinate (100 mg, 0.456 mmol), following a procedure similar to that described for the synthesis of methyl 4-(tetrahydro-2H-thiopyran-4-yl)benzoate and was isolated as an off-white solid.

Yield 50 mg (50%). ¹H NMR (400 MHz, DMSO) δ 8.65 (d, J=2.4 Hz, 1H), 8.01 (d, J=8.0 Hz, 1H), 7.89 (dd, J=2.4, 8.0 Hz, 1H), 3.03-3.94 (m, 2H), 3.87 (s, 3H), 3.51-3.41 (m, 2H), 3.02-2.90 (m, 1H), 1.77-1.68 (m, 4H). m/z: [ESI⁺] 222 (M+H)⁺.

### Synthesis of 5-(tetrahydro-2H-pyran-4-yl)picolinic acid

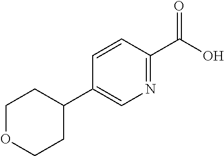 [SMILES: O=C(O)c1ccc(C2CCOCC2)cn1]

Compound 5-(tetrahydro-2H-pyran-4-yl)picolinic acid was prepared from methyl 5-(tetrahydro-2H-pyran-4-yl)picolinate (50 mg, 0.226 mmol) and lithium hydroxide monohydrate (38 mg, 0.906 mmol), following a procedure similar to that described for the synthesis of 4-(1,1-dioxidotetrahydro-2H-thiopyran-4-yl)benzoic acid, and was isolated as an off-white solid.

Yield 39 mg (83%). ¹H NMR (400 MHz, CDCl₃) δ 8.61 (d, J=2.0 Hz, 1H), 8.22 (d, J=8.0 Hz, 1H), 7.82 (dd, J=2.0, 8.0 Hz, 1H), 4.21-4.09 (m, 2H), 3.58 (dt, J=3.2, 11.6 Hz, 2H), 3.03-2.87 (m, 1H), 1.97-1.76 (m, 4H). Carboxylic acid OH proton not observed. m/z: [ESI⁺] 208 (M+H)⁺.

### Synthesis of benzyl bis(2-oxoethyl)carbamate

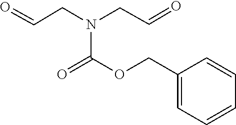 [SMILES: O=CCN(CC=O)C(=O)OCc1ccccc1]

To a stirred mixture of benzyl 2,5-dihydro-1H-pyrrole-1-carboxylate (3.80 g, 18.70 mmol), sodium periodate (16.00 g, 74.80 mmol) and 2,6-lutidine (4.01 g, 37.42 mmol) in ethyl acetate (40 mL) and water (40 mL) was added potassium osmate(VI) dihydrate (0.34 g, 0.92 mmol) at 0° C. The resulting mixture was stirred for 3 h at room temperature. The resulting mixture was extracted with ethyl acetate (3×200 mL). The combined organic layers were washed with brine (100 mL) and dried over anhydrous Na₂SO₄. After filtration, the filtrate was concentrated under reduced pressure. The residue was purified by silica gel column chromatography, eluting with 0-9% methanol in DCM to afford benzyl bis(2-oxoethyl)carbamate as a dark brown semi-solid.

Yield 3.30 g (75%). ¹H NMR (400 MHz, DMSO) δ 7.44-7.25 (m, 5H), 5.09 (s, 2H), 3.35 (s, 4H). Aldehyde CH protons not observed. No MS signal.

### Synthesis of benzyl 4-(3-(methoxycarbonyl)bicyclo[1.1.1]pentan-1-yl)piperazine-1-carboxylate

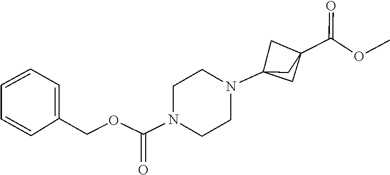 [SMILES: COC(=O)C12CC(N3CCN(C(=O)OCc4ccccc4)CC3)(C1)C2]

To a stirred mixture of methyl 3-aminobicyclo[1.1.1]pentane-1-carboxylate hydrochloride (0.80 g, 4.50 mmol) and benzyl bis(2-oxoethyl)carbamate (1.17 g, 4.97 mmol) in methanol (10 mL) was added acetic acid (0.95 g, 15.82 mmol) and sodium cyanoborohydride (0.99 g, 15.75 mmol) at 0° C. The resulting mixture was stirred for 16 h at room temperature. The reaction was quenched by the addition of water (50 mL) and extracted with ethyl acetate (3×100 mL). The combined organic layers were washed with brine (100 mL) and dried over anhydrous Na₂SO₄. After filtration, the filtrate was concentrated under reduced pressure. The residue was purified by reverse phase flash chromatography with the following conditions: Column: WelHash™ C18-I, 20-40 μm, 330 g; Eluent A: water (plus 10 mmol/L NH₄HCO₃); Eluent B: acetonitrile; Gradient: 40%-60% B in 20 min; How rate: 60 mL/min; Detector: UV 220/254 nm. The desired fractions were collected and concentrated under reduced pressure to afford benzyl 4-(3-(methoxycarbonyl)bicyclo[1.1.1]pentan-1-yl)piperazine-1-carboxylate as an off-white solid.

Yield 0.58 g (37%). ¹H NMR (400 MHz, DMSO) δ 7.42-7.28 (m, 5H), 5.08 (s, 2H), 3.61 (s, 3H), 3.45-3.35 (m, 4H), 2.40-2.27 (m, 4H), 1.96 (s, 6H). m/z: [ESI⁺] 345 (M+H)⁺.

### Synthesis of methyl 3-(piperazin-1-yl)bicyclo[1.1.1]pentane-1-carboxylate

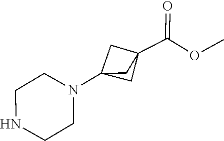

A mixture of benzyl 4-(3-(methoxycarbonyl)bicyclo[1.1.1]pentan-1-yl)piperazine-1-carboxylate (0.58 g, 1.68 mmol) and 10% wt. palladium on charcoal (0.18 g) in ethyl acetate (10 mL) was stirred for 16 h at room temperature under a hydrogen atmosphere (1.5 atm). The resulting mixture was filtered and the filter cake was washed with ethyl acetate (3×50 mL). The combined washings and filtrate were concentrated under reduced pressure to afford methyl 3-(piperazin-1-yl)bicyclo[1.1.1]pentane-1-carboxylate as a yellow oil.

Yield 0.27 g (76%). ¹H NMR (400 MHz, DMSO) δ 3.60 (s, 3H), 2.65 (t, J=4.8 Hz, 4H), 2.26 (t, J=4.8 Hz, 4H), 1.93 (s, 6H). Aliphatic NH not observed. m/z: [ESI⁺] 211 (M+H)⁺.

### Synthesis of methyl 3-(4-(methylsulfonyl)piperazin-1-yl)bicyclo[1.1.1]pentane-1-carboxylate

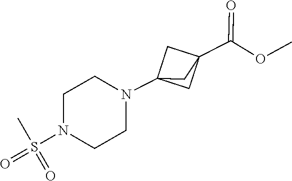 [SMILES: COC(=O)C12CC(N3CCN(S(C)(=O)=O)CC3)(C1)C2]

To a stirred solution of methyl 3-(piperazin-1-yl)bicyclo[1.1.1]pentane-1-carboxylate (0.27 g, 1.28 mmol) and triethylamine (0.39 g, 3.85 mmol) in DCM (5 ml) was added methanesulfonyl chloride (0.22 g, 1.92 mmol) dropwise at 0° C. under a nitrogen atmosphere. The resulting mixture was stirred for 4 h at room temperature under a nitrogen atmosphere. The resulting mixture was concentrated under reduced pressure. The residue was purified by reverse phase flash chromatography with the following conditions: Column: WelFlash TM C18-I, 20-40 μm, 120 g; Eluent A: water (plus 10 mmol/L NH₄CO₃); Eluent B: acetonitrile; Gradient: 40%-60% B in 20 min; How rate: 60 m/min; Detector: UV 220/254 nm. The desired fractions were collected and concentrated under reduced pressure to afford methyl 3-(4-(methylsulfonyl)piperazin-1-yl)bicyclo[1.1.1]pentane-1-carboxylate as a light yellow solid.

Yield 0.26 g (70%). ¹H NMR (400 MHz, DMSO) δ 3.61 (s, 3H), 3.10 (t, J=4.8 Hz, 4H), 2.87 (s, 3H), 2.46 (t, J=4.8 Hz, 4H), 1.97 (s, 6H). m/z: [ESI⁺] 289 (M+H)⁺.

### Synthesis of 3-(4-(methylsulfonyl)piperazin-1-yl)bicyclo[1.1.1]pentane-1-carboxylic acid

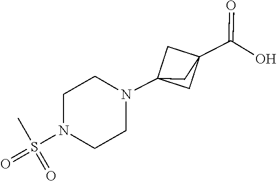 [SMILES: CS(=O)(=O)N1CCN(C23CC(C(=O)O)(C2)C3)CC1]

Compound 3-(4-(methylsulfonyl)piperazin-1-yl)bicyclo[1.1.1]pentane-1-carboxylic acid was prepared from methyl 3-(4-(methylsulfonyl)piperazin-1-yl)bicyclo[1.1.1]pentane-1-carboxylate (260 mg, 0.902 mmol) and lithium hydroxide monohydrate (151 mg, 3.598 mmol), following a procedure similar to that described for the synthesis of 4-(1,1-dioxidotetrahydro-2H-thiopyran-4-yl)benzoic acid and was isolated as an off-white semi-solid.

Yield 210 mg (85%). m/z: [ESI⁺] 275 (M+H)⁺.

### Synthesis of methyl 3-morpholinobicyclo[1.1.1]pentane-1-carboxylate

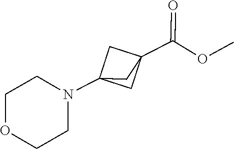

To a stirred mixture of methyl 3-aminobicyclo[1.1.1]pentane-1-carboxylate hydrochloride (0.50 g, 2.81 mmol) and potassium carbonate (1.95 g, 14.11 mmol) in acetonitrile (10 mL) was added 1-bromo-2-(2-bromoethoxy)ethane (1.96 g, 8.45 mmol) at room temperature under a nitrogen atmosphere. The resulting mixture was stirred for 16 h at 80° C. under a nitrogen atmosphere. The resulting mixture was cooled to room temperature and filtered and the filter cake was washed with DCM (3×20 mL). The combined washings and filtrate were concentrated under reduced pressure. The residue was purified by silica gel column chromatography, eluting with 0-9% methanol in DCM to afford methyl 3-morpholinobicyclo[1.1.1]pentane-1-carboxylate as a yellow oil.

Yield 0.37 g (62%). ¹H NMR (400 MHz, DMSO) δ 3.61 (s, 3H), 3.59-3.54 (m, 4H), 2.38-2.29 (m, 4H), 1.96 (s, 6H). m/z: [ESI⁺] 212 (M+H)⁺.

### Synthesis of 3-morpholinobicyclo[1.1.1]pentane-1-carboxylic acid

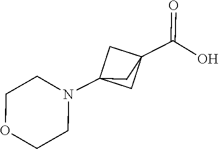 [SMILES: O=C(O)C12CC(N3CCOCC3)(C1)C2]

Compound 3-morpholinobicyclo[1.1.1]pentane-1-carboxylic acid was prepared from methyl 3-morpholinobicyclo[1.1.1]pentane-1-carboxylate (0.37 g, 1.75 mmol) and lithium hydroxide monohydrate (0.22 g, 5.24 mmol), following a procedure similar to that described for the synthesis of 4-(1,1-dioxidotetrahydro-2H-thiopyran-4-yl)benzoic acid and was isolated as an off-white solid.

Yield 0.30 g (87%). ¹H NMR (400 MHz, DMSO) δ 3.59-3.53 (m, 4H), 2.36-2.27 (m, 4H), 1.79 (s, 6H). Carboxylic acid proton not observed. m/z: [ESI⁺] 198 (M+H)⁺.

### Synthesis of methyl (1r,3r)-3-morpholinocyclobutane-1-carboxylate

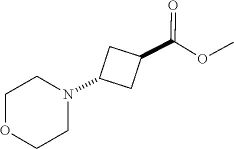

Compound methyl (1r,3r)-3-morpholinocyclobutane-1-carboxylate was prepared from methyl (1r,3r)-3-aminocyclobutane-1-carboxylate hydrochloride (0.50 g, 3.02 mmol) and 1-bromo-2-(2-bromoethoxy)ethane (2.10 g, 9.05 mmol) following a procedure similar to that described for the synthesis of methyl 3-morpholinobicyclo[1.1.1]pentane-1-carboxylate and was isolated as an off-white solid.

Yield 0.60 g (99%). ¹H NMR (400 MHz, DMSO) δ 3.99 (dd, J=3.2, 12.4 Hz, 2H), 3.90 (q, J=8.4 Hz, 111), 3.74-3.61 (m, 2H), 3.65 (s, 3H), 3.40-3.30 (m, 211), 3.18-3.09 (m, 1H), 2.98-2.85 (m, 2H), 2.70-2.56 (m, 2H), 2.46-2.36 (m, 2H). m/z: [ESI⁺] 200 (M+H)⁺.

### Synthesis of (1r,3r)-3-morpholinocyclobutane-1-carboxylic acid

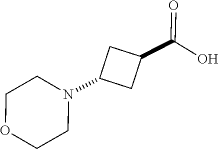

Compound (1r,3r)-3-morpholinocyclobutane-1-carboxylic acid was prepared from methyl (1r,3r)-3-morpholinocyclobutane-1-carboxylate (0.60 g, 3.01 mmol) and lithium hydroxide monohydrate (0.38 g, 9.06 mmol), following a procedure similar to that described for the synthesis of 4-(1,1-dioxidotetrahydro-2H-thiopyran-4-yl)benzoic acid and was isolated as a light yellow semi-solid.

Yield 0.4 g (72%). ¹H NMR (400 MHz, DMSO) δ 12.51 (br s, 1H), 3.99-3.74 (m, 5H), 3.27 (d, J=12.4 Hz, 2H), 3.08-2.96 (m, 1H), 2.94-2.81 (m, 2H), 2.79-2.64 (m, 2H), 2.41-2.28 (m, 2H). m/z: [ESI⁺] 186 (M+H)⁺.

### Synthesis of methyl 5-(2-methyl-1-oxo-2,8-diazaspiro[4.5]decan-8-yl)picolinate

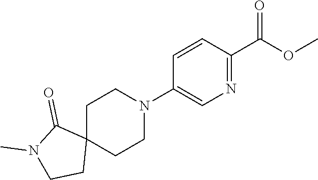

To a stirred mixture of methyl 5-bromopicolinate (0.63 g, 2.92 mmol) and 2-methyl-2,8-diazaspiro[4.5]decan-1-one hydrochloride (0.50 g, 2.44 mmol) in DMF (12 mL) were added 9,9-dimethyl-4,5-bis(diphenylphosphino)xanthene (0.28 g, 0.48 mmol), palladium (II) acetate (55 mg, 0.245 mmol) and cesium carbonate (2.39 g, 7.34 mmol) at room temperature under a nitrogen atmosphere. The resulting mixture was stirred overnight at 100° C. The resulting mixture was cooled to room temperature, filtered and the filter cake washed with DMF (3×2 mL). The combined washings and filtrate were concentrated under reduced pressure. The residue was purified by reverse phase flash chromatography with the following conditions: Column: WelFlash TM C18-I, 20-40 μm, 330 g; Eluent A: water (plus 10 mmol/L NH₄HCO₃); Eluent B: acetonitrile; Gradient: 25%-45% B in 25 min; How rate: 80 m/min; Detector: UV 220/254 nm. The desired fractions were collected and concentrated under reduced pressure to afford methyl 5-(2-methyl-1-oxo-2,8-diazaspiro[4.5]decan-8-yl)picolinate as an off-white solid.

Yield 440 mg (59%). ¹H NMR (400 MHz, DMSO) δ 8.39 (d, J=2.8 Hz, 1H), 7.87 (d, J=8.8 Hz, 1H), 7.36 (dd, J=2.8, 8.8 Hz, 1H), 3.91 (dt, J=4.0, 13.6 Hz, 2H), 3.81 (s, 3H), 3.35-3.25 (m, 5H), 3.13-3.01 (m, 2H), 1.99 (t, J=6.8 Hz, 2H), 1.78-1.70 (m, 2H), 1.45 (dd, J=4.0, 13.6 Hz, 2H). m/z: [ESI⁺] 304 (M+H)⁺.

### Synthesis of 5-(2-methyl-1-oxo-2,8-diazaspiro[4.5]decan-8-yl)picolinic acid

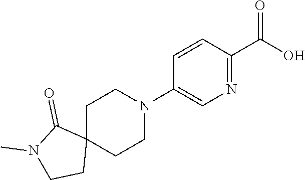 [SMILES: CN1CCC2(CCN(c3ccc(C(=O)O)nc3)CC2)C1=O]

Compound 5-(2-methyl-1-oxo-2,8-diazaspiro[4.5]decan-8-yl)picolinic acid was prepared from methyl 5-(2-methyl-1-oxo-2,8-diazaspiro[4.5]decan-8-yl)picolinate (0.44 g, 1.45 mmol) and lithium hydroxide monohydrate (0.18 g, 4.29 mmol), following a procedure similar to that described for the synthesis of 4-(1,1-dioxidotetrahydro-2H-thiopyran-4-yl)benzoic acid, and was isolated as an off-white solid.

Yield 0.3 g (71%). ¹H NMR (400 MHz, DMSO) δ 8.28 (d, J=2.8 Hz, 1H), 7.81 (d, J=8.8 Hz, 1H), 7.33 (dd, J=2.8, 8.8 Hz, 1H), 3.79 (d, J=12.8 Hz, 2H), 3.31 (t, J=6.8 Hz, 2H), 2.98 (t, J=12.4 Hz, 2H), 2.74 (s, 3H), 1.97 (t, J=6.8 Hz, 2H), 1.74 (dt, J=4.0, 12.8 Hz, 2H), 1.42 (d, J=13.2 Hz, 2H). Carboxylic acid proton not observed. m/z: [ESI⁺] 290 (M+H)⁺.

### Synthesis of methyl 5-(2-oxa-7-azaspiro[3.5]nonan-7-yl)picolinate

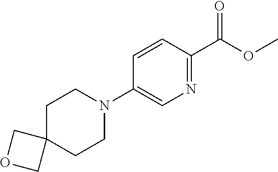 [SMILES: COC(=O)c1ccc(N2CCC3(CC2)COC3)cn1]

Compound methyl 5-(2-oxa-7-azaspiro[3.5]nonan-7-yl)picolinate was prepared from methyl 5-bromopicolinate (1.80 g, 8.33 mmol) and 2-oxa-7-azaspiro[3.5]nonane (1.20 g, 9.43 mmol) following a procedure similar to that described for the synthesis of methyl 5-(2-methyl-1-oxo-2,8-diazaspiro[4.5]decan-8-yl)picolinate, and was isolated as an off-white solid.

Yield 1.40 g (64%). ¹H NMR (400 MHz, CDCl₃) δ 8.35 (d, J=2.8 Hz, 1H), 7.99 (d, J=8.8 Hz, 1H), 7.16 (dd, J=2.8, 8.8 Hz, 1H), 4.50 (s, 4H), 3.96 (s, 3H), 3.37-3.27 (m, 4H), 2.05-1.97 (m, 4H). m/z: [ESI⁺] 263 (M+H)⁺.

### Synthesis of 5-(2-oxa-7-azaspiro[3.5]nonan-7-yl)picolinic acid

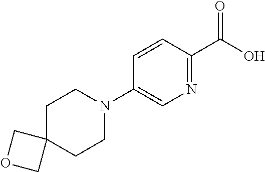 [SMILES: O=C(O)c1ccc(N2CCC3(CC2)COC3)cn1]

Compound 5-(2-oxa-7-azaspiro[3.5]nonan-7-yl)picolinic acid was prepared from methyl 5-(2-oxa-7-azaspiro[3.5]nonan-7-yl)picolinate (1.40 g, 5.34 mmol) and lithium hydroxide monohydrate (0.67 g, 15.97 mmol), following a procedure similar to that described for the synthesis of 4-(1,1-dioxidotetrahydro-2H-thiopyran-4-yl)benzoic acid and was isolated as an off-white solid.

Yield 1.20 g (91%). ¹H NMR (400 MHz, DMSO) δ 8.29 (d, J=2.8 Hz, 1H), 7.80 (d, J=8.8 Hz, 1H), 7.33 (dd, J=2.8, 8.8 Hz, 1H), 4.34 (s, 4H), 3.27 (t, J=5.6 Hz, 4H), 1.86 (t, J=5.6 Hz, 4H). Carboxylic acid proton not observed. m/z: [ESI⁺] 249 (M+H)⁺.

### Synthesis of methyl 2-methoxy-4-morpholinobenzoate

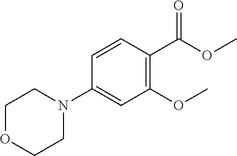 [SMILES: COC(=O)c1ccc(N2CCOCC2)cc1OC]

Compound methyl 2-methoxy-4-morpholinobenzoate was prepared from methyl 4-bromo-2-methoxybenzoate (500 mg, 2.04 mmol) and morpholine (267 mg, 3.07 mmol) following a procedure similar to that described for the synthesis of methyl 5-(2-methyl-1-oxo-2,8-diazaspiro[4.5]decan-8-yl)picolinate and was isolated as an off-white solid.

Yield 300 mg (59%). ¹H NMR (400 MHz, DMSO) δ 7.63 (d, J=8.8 Hz, 1H), 6.54 (dd, J=2.4, 8.8 Hz, 1H), 6.51 (d, J=2.4 Hz, 1H), 3.80 (s, 3H), 3.76-3.71 (m, 4H), 3.70 (s, 3H), 3.30-3.25 (m, 4H). m/z: [ESI⁺] 252 (M+H)⁺.

### Synthesis of 2-methoxy-4-morpholinobenzoic acid

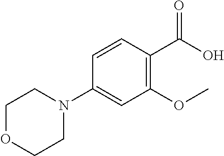 [SMILES: COc1cc(N2CCOCC2)ccc1C(=O)O]

Compound 2-methoxy-4-morpholinobenzoic acid was prepared from methyl 2-methoxy-4-morpholinobenzoate (300 mg, 1.19 mmol) and lithium hydroxide monohydrate (200 mg, 4.77 mmol), following a procedure similar to that described for the synthesis of 4-(1,1-dioxidotetrahydro-2H-thiopyran-4-yl)benzoic acid and was isolated as an off-white solid.

Yield 250 mg (88%). ¹H NMR (400 MHz, DMSO) δ 11.81 (br s, 1H), 7.64 (d, J=8.8 Hz, 1H), 6.57-6.47 (m, 2H), 3.81 (s, 3H), 3.73 (t, J=4.8 Hz, 4H), 3.26 (t, J=4.8 Hz, 4H). m/z: [ESI⁺] 238 (M+H)⁺.

### Synthesis of methyl 5-((1-methylpiperidin-4-yl)oxy)picolinate

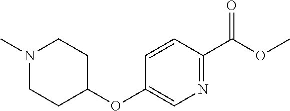

To a solution of methyl 5-hydroxypicolinate (1.53 g, 9.99 mmol) in THF (50 mL) were added 1-methylpiperidin-4-ol (2.30 g, 19.97 mmol), triphenylphosphine (3.93 g, 14.98 mmol) and DIAD (3.03 g, 14.98 mmol) at 0° C. under a nitrogen atmosphere. The resulting mixture was stirred overnight at room temperature under a nitrogen atmosphere. The resulting mixture was filtered and the filtrate was concentrated under reduced pressure. The residue was purified by reverse phase flash chromatography with the following conditions: Column: WelFlash TM C18-L, 20-40 μm, 330 g; Eluent A: water (plus 10 mmol/L NH₄HCO₃); Eluent B: acetonitrile; Gradient: 35%-55% B in 25 min; Flow rate: 80 mL/min; Detector: UV 220/254 nm. The desired fractions were collected and concentrated under reduced pressure to afford methyl 5-((1-methylpiperidin-4-yl)oxy)picolinate as an off-white solid.

Yield 1.31 g (52%). ¹H NMR (400 MHz, DMSO) δ 8.37 (d, J=2.8 Hz, 1H), 8.01 (d, J=8.8 Hz, 1H), 7.56 (dd, J=2.8, 8.8 Hz, 1H), 4.59 (tt, J=4.0, 8.0 Hz, 1H), 3.84 (s, 3H), 2.66-2.55 (m, 2H), 2.26-2.13 (m, 5H), 2.02-1.89 (m, 2H), 1.73-1.59 (m, 2H). m/z: [ESI⁺] 251 (M+H)⁺.

### Synthesis of 5-((1-methylpiperidin-4-yl)oxy)picolinic acid

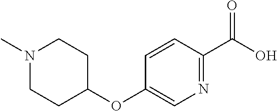 [SMILES: CN1CCC(Oc2ccc(C(=O)O)nc2)CC1]

Compound 5-((1-methylpiperidin-4-yl)oxy)picolinic acid was prepared from methyl 5-((1-methylpiperidin-4-yl)oxy)picolinate (1.31 g, 5.23 mmol) and lithium hydroxide monohydrate (0.88 g, 20.97 mmol), following the procedure similar to that described for the synthesis of 4-(1,1-dioxidotetrahydro-2H-thiopyran-4-yl)benzoic acid and was isolated as an off-white solid.

Yield 1.15 g (93%). ¹H NMR (400 MHz, DMSO) δ 8.37 (d, J=2.8 Hz, 1H), 8.00 (d, J=8.8 Hz, 1H), 7.58 (dd, J=2.8, 8.8 Hz, 1H), 4.80-4.66 (m, 1H), 4.19-4.04 (m, 2H), 3.07-2.93 (m, 2H), 2.51 (s, 3H), 2.17-2.03 (m, 2H), 1.94-1.77 (m, 2H). Carboxylic acid proton not observed. m/z: [ESI⁺] 237 (M+H)⁺.

### Synthesis of 2-chloro-4-morpholinobenzaldehyde

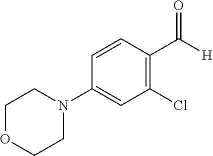 [SMILES: O=Cc1ccc(N2CCOCC2)cc1Cl]

To a stirred solution of 2-chloro-4-fluorobenzaldehyde (5.00 g, 31.53 mmol) in DMF (100 mL) were added morpholine (5.51 g, 63.25 mmol) and potassium carbonate (8.75 g, 63.31 mmol) at room temperature. The resulting mixture was stirred for 16 h at 120° C. under a nitrogen atmosphere. The resulting mixture was cooled to room temperature and diluted with water (300 mL). The resulting mixture was extracted with ethyl acetate (3×300 mL). The combined organic layers were washed with brine (200 mL) and dried over anhydrous Na₂SO₄. After filtration, the filtrate was concentrated under reduced pressure. The residue was purified by silica gel column chromatography, eluting with 0-50% ethyl acetate in petroleum ether to afford 2-chloro-4-morpholinobenzaldehyde as an off-white solid.

Yield 4.72 g (66%). ¹H NMR (400 MHz, DMSO) δ 10.08 (s, 1H), 7.70 (d, J=9.6 Hz, 1H), 7.05-6.96 (m, 2H), 3.75-3.67 (m, 4H), 3.42-3.36 (m, 4H). m/z: [ESI⁺] 226, 228 (M+H)⁺.

### Synthesis of 2-chloro-4-morpholinobenzoic acid

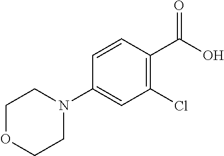 [SMILES: O=C(O)c1ccc(N2CCOCC2)cc1Cl]

To a stirred mixture of 2-chloro-4-morpholinobenzaldehyde (1.00 g, 4.43 mmol) and 2-methylbut-2-ene (8.00 mL) in tert-butanol (40 mL) were added a solution of sodium dihydrogen phosphate dihydrate (0.86 g, 5.51 mmol) in water (10 ml) and a solution of sodium chlorite (0.60 g, 6.65 mmol) in water (5 mL) dropwise at room temperature. The resulting mixture was stirred overnight at room temperature. The resulting mixture was concentrated under reduced pressure. The residue was diluted with water (50 mL) and was acidified to pH 5 with a solution of aqueous 1 M HCl. The resulting mixture was extracted with ethyl acetate (5×50 mL). The combined organic layers were dried over anhydrous Na₂SO₄. After filtration, the filtrate was concentrated under reduced pressure to afford 2-chloro-4-morpholinobenzoic acid as an off-white solid.

Yield 0.85 g (79%). ¹H NMR (400 MHz, DMSO) δ 12.75 (br s, 1H), 7.78 (d, J=8.8 Hz, 1H), 6.98 (d, J=2.4 Hz, 1H), 6.93 (dd, J=2.4, 8.8 Hz, 1H), 3.74-3.68 (m, 4H), 3.29-3.25 (m, 4H). m/z: [ESI⁺] 242, 244 (M+H)⁺.

### Synthesis of 2-methyl-4-morpholinobenzaldehyde

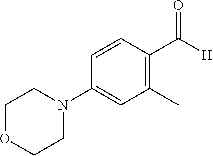

Compound 2-methyl-4-morpholinobenzaldehyde was prepared from 4-fluoro-2-methylbenzaldehyde (1.00 g, 7.24 mmol) and morpholine (1.30 g, 14.92 mmol) following a procedure similar to that described for the synthesis of 2-chloro-4-morpholinobenzaldehyde, and was isolated as an off-white solid.

Yield 1.22 g (82%). ¹H NMR (400 MHz, DMSO) δ 9.94 (s, 1H), 7.65 (d, J=8.8 Hz, 1H), 6.89 (dd, J=2.4, 8.8 Hz, 1H), 6.81 (d, J=2.4 Hz, 1H), 3.79-3.68 (m, 4H), 3.34-3.30 (m, 4H), 2.55 (s, 3H). m/z: [ESI⁺] 206 (M+H)⁺.

### Synthesis of 2-methyl-4-morpholinobenzoic acid

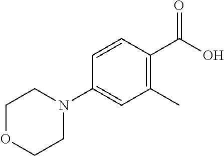 [SMILES: Cc1cc(N2CCOCC2)ccc1C(=O)O]

Compound 2-methyl-4-morpholinobenzoic acid was prepared from 2-methyl-4-morpholinobenzaldehyde (1.21 g, 5.89 mmol) following a procedure similar to that described for the synthesis of 2-chloro-4-morpholinobenzoic acid and was isolated as an off-white solid.

Yield 882 mg (68%). ¹H NMR (400 MHz, DMSO) δ 12.26 (br s, 1H), 7.77 (d, J=9.6 Hz, 1H), 6.81-6.77 (m, 2H), 3.76-3.68 (m, 4H), 3.27-3.18 (m, 4H), 2.51 (s, 3H). m/z: [ESI⁺] 222 (M+H)⁺.

### Synthesis of 4-morpholino-2-(trifluoromethyl)benzaldehyde

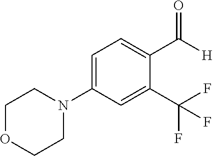 [SMILES: O=Cc1ccc(N2CCOCC2)cc1C(F)(F)F]

Compound 4-morpholino-2-(trifluoromethyl)benzaldehyde was prepared from 4-fluoro-2-(trifluoromethyl)benzaldehyde (1.00 g, 5.21 mmol) and morpholine (0.90 g, 10.33 mmol) following a procedure similar to that described for the synthesis of 2-chloro-4-morpholinobenzaldehyde and was isolated as an off-white solid.

Yield 916 mg (68%). ¹H NMR (400 MHz, DMSO) δ 9.99 (q, J=2.0 Hz, 1H), 7.95 (d, J=8.8 Hz, 1H), 7.40-7.16 (m, 2H), 3.84-3.69 (m, 4H), 3.48-3.40 (m, 4H). ¹⁹F NMR (376 MHz, DMSO) δ −55.81. m/z: [ESI⁺]260 (M+H)⁺.

### Synthesis of 4-morpholino-2-(trifluoromethyl)benzoic acid

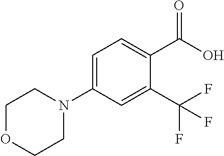 [SMILES: O=C(O)c1ccc(N2CCOCC2)cc1C(F)(F)F]

Compound 4-morpholino-2-(trifluoromethyl)benzoic acid was prepared from 4-morpholino-2-(trifluoromethyl)benzaldehyde (0.92 g, 3.55 mmol) following the procedure similar to that described for the synthesis of 2-chloro-4-morpholinobenzoic acid, and was isolated as a yellow solid.

Yield 585 mg (60%). m/z: [ESI⁺] 276 (M+H)⁺.

### Synthesis of methyl (1r,3r)-3-((4-(2-chlorophenyl)thiazol-2-yl)carbamoyl)cyclobutane-1-carboxylate

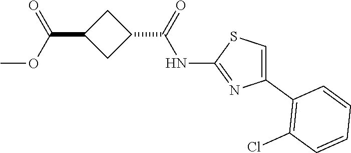

To a stirred solution of 3-(methoxycarbonyl)cyclobutane-1-carboxylic acid (1.00 g, 6.32 mmol), 4-(2-chlorophenyl)thiazol-2-amine (1.47 g, 6.98 mmol) and DIPEA (1.63 g, 12.61 mmol) in DMF (20 mL) was added HATU (3.61 g, 9.49 mmol) portionwise at 0° C. under a nitrogen atmosphere. The resulting solution was stirred for 16 h at room temperature under a nitrogen atmosphere. The solution was diluted with water (60 ml.) and extracted with ethyl acetate (2×30 mL). The combined organic layers were washed with brine (20 mL) and dried over anhydrous Na₂SO₄. After filtration, the filtrate was concentrated under reduced pressure. The residue was purified by silica gel column chromatography, eluting with 0-30% ethyl acetate in petroleum ether to afford methyl (1r,3r)-3-((4-(2-chlorophenyl)thiazol-2-yl)carbamoyl)cyclobutane-1-carboxylate (A) (assumed) and methyl (1s,3s)-3-((4-(2-chlorophenyl)thiazol-2-yl)carbamoyl)cyclobutane-1-carboxylate (B) (assumed) as off-white solids.

A: Yield 0.81 g (37%). ¹H NMR (400 MHz, DMSO) δ 12.23 (br s, 1H), 7.83 (dd, J=2.0, 7.6 Hz, 1H), 7.61 (s, 1H), 7.55 (dd, J=1.6, 7.6 Hz, 1H), 7.46-7.33 (m, 2H), 3.65 (s, 3H), 3.48-3.36 (m, 1H), 3.25-3.15 (m, 111), 2.55-2.38 (m, 4H). m/z: [ESI⁺] 351 (M+H)⁺.
B: Yield 1.00 g (45%). ¹H NMR (400 MHz, DMSO) δ 12.27 (br s, 1H), 7.83 (dd, J=2.0, 7.6 Hz, 1H), 7.60 (s, 1H), 7.54 (dd, J=1.6, 7.6 Hz, 1H), 7.45-7.33 (m, 2H), 3.62 (s, 3H), 3.38-3.26 (m, 1H), 3.23-3.13 (m, 1H), 2.48-2.34 (m, 4H). m/z: [ESI⁺] 351 (M+H)⁺.

### Synthesis of (1r,3r)-3-((4-(2-chlorophenyl)thiazol-2-yl)carbamoyl)cyclobutane-1-carboxylic acid

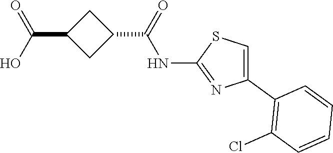 [SMILES: O=C(O)[C@H]1C[C@H](C(=O)Nc2nc(-c3ccccc3Cl)cs2)C1]

Compound (1r,3r)-3-((4-(2-chlorophenyl)thiazol-2-yl)carbamoyl)cyclobutane-1-carboxylic acid was prepared from methyl (1r,3r)-3-((4-(2-chlorophenyl)thiazol-2-yl)carbamoyl)cyclobutane-1-carboxylate (810 mg, 2.309 mmol) and lithium hydroxide monohydrate (388 mg, 9.247 mmol), following a procedure similar to that described for the synthesis of 2-chloro-4-morpholinobenzoic acid and was isolated as an off-white solid.

Yield 700 mg (90%). ¹H NMR (400 MHz, DMSO) δ 12.21 (br s, 2H), 7.83 (dd, J=2.0, 7.6 Hz, 1H), 7.61 (s, 1H), 7.55 (dd, J=1.6, 7.6 Hz, 1H), 7.46-7.35 (m, 2H), 3.46-3.36 (m, 1H), 3.14-3.04 (m, 1H), 2.49-2.35 (m, 4H). m/z: [ESI⁺] 337, 339 (M+H)⁺.

### Synthesis of methyl 4-((4-(2-chlorophenyl)thiazol-2-yl)carbamoyl)benzoate

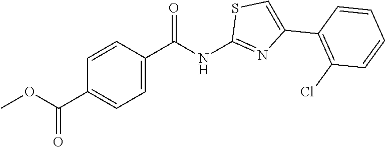 [SMILES: COC(=O)c1ccc(C(=O)Nc2nc(-c3ccccc3Cl)cs2)cc1]

Compound methyl 4-((4-(2-chlorophenyl)thiazol-2-yl)carbamoyl)benzoate was prepared from 4-(methoxycarbonyl)benzoic acid (3.00 g, 16.65 mmol) and 4-(2-chlorophenyl)thiazol-2-amine (2.46 g, 11.68 mmol) following a procedure similar to that described for the synthesis of (1r,3r)-3-((4-(2-chlorophenyl)thiazol-2-yl)carbamoyl)cyclobutane-1-carboxylate and was isolated as an off-white solid.

Yield 2.50 g (57%). ¹H NMR (400 MHz, DMSO) δ 13.03 (br s, 1H), 8.24 (d, J=8.4 Hz, 2H), 8.11 (d, J=8.4 Hz, 2H), 7.91 (dd, J=2.0, 7.6 Hz, 1H), 7.72 (s, 1H), 7.58 (dd, J=1.6, 7.6 Hz, 1H), 7.51-7.39 (m, 2H), 3.91 (s, 3H). m/z: [ESI⁺] 373, 375 (M+H)⁺.

### Synthesis of 4-((4-(2-chlorophenyl)thiazol-2-yl)carbamoyl)benzoic acid

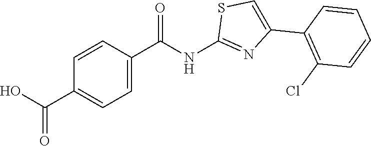 [SMILES: O=C(O)c1ccc(C(=O)Nc2nc(-c3ccccc3Cl)cs2)cc1]

Compound 4-((4-(2-chlorophenyl)thiazol-2-yl)carbamoyl)benzoic acid was prepared from methyl 4-((4-(2-chlorophenyl)thiazol-2-yl)carbamoyl)benzoate (500 mg, 1.34 mmol) and lithium hydroxide monohydrate (225 mg, 5.36 mmol), following a procedure similar to that described for the synthesis of 4-(1,1-dioxidotetrahydro-2H-thiopyran-4-yl)benzoic acid and was isolated as a red solid.

Yield 400 mg (83%). ¹H NMR (400 MHz, DMSO) δ 13.30 (br s, 1H), 13.09 (br s, 1H), 8.22 (d, J=8.4 Hz, 2H), 8.09 (d, J=8.4 Hz, 2H), 7.91 (dd, J=1.6, 7.6 Hz, 1H), 7.71 (s, 1H), 7.58 (d, J=7.6 Hz, 1H), 7.50-7.36 (m, 2H). m/z: [ESI⁺] 359, 361 (M+H)⁺.

### Synthesis of tert-butyl 4-(6-(methoxycarbonyl)pyridin-3-yl)piperazine-1-carboxylate

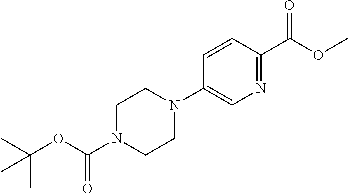 [SMILES: COC(=O)c1ccc(N2CCN(C(=O)OC(C)(C)C)CC2)cn1]

Compound tert-butyl 4-(6-(methoxycarbonyl)pyridin-3-yl)piperazine-1-carboxylate was prepared from methyl 5-bromopicolinate (10.00 g, 46.29 mmol) and tert-butyl piperazine-1-carboxylate (12.93 g, 69.42 mmol) following a procedure similar to that described for the synthesis of methyl 5-(2-methyl-1-oxo-2,8-diazaspiro[4.5]decan-8-yl)picolinate and was isolated as a yellow solid.

Yield 7.20 g (48%). ¹H NMR (400 MHz, CDCl₃) δ 8.33 (d, J=2.8 Hz, 1H), 8.00 (d, J=8.8 Hz, 1H), 7.15 (dd, J=2.8, 8.8 Hz, 1H), 3.95 (s, 3H), 3.66-3.56 (m, 4H), 3.38-3.29 (m, 4H), 1.47 (s, 9H). m/z: [ESI⁺] 322 (M+H)⁺.

### Synthesis of methyl 5-(piperazin-1-yl)picolinate hydrochloride

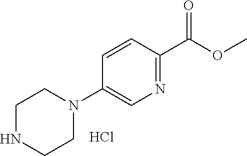 [SMILES: COC(=O)c1ccc(N2CCNCC2)cn1 ; Cl]

tert-Butyl 4-(6-(methoxycarbonyl)pyridin-3-yl)piperazine-1-carboxylate (25.00 g, 77.79 mmol) was dissolved in a 4 M solution of HCl in 1,4-dioxane (250 mL). The resulting solution was stirred for 6 h at room temperature under a nitrogen atmosphere. The precipitated solid was collected by filtration. The filter cake was washed with diethyl ether (6×80 mL) and dried under reduced pressure to afford methyl 5-(piperazin-1-yl)picolinate hydrochloride as a yellow solid.

Yield 19.60 g (98%). ¹H NMR (400 MHz, DMSO) δ 9.93 (br s, 2H, NH₂+), 8.44 (d, J=2.8 Hz, 1H), 8.05 (d, J=8.8 Hz, 1H), 7.70 (dd, J=2.8, 8.8 Hz, 1H), 3.88 (s, 3H), 3.76 (t, J=5.6 Hz, 4H), 3.20 (t, J=5.6 Hz, 4H). m/z: [ESI⁺] 222 (M+H)⁺.

### Synthesis of methyl 5-(4-(methylsulfonyl)piperazin-1-yl)picolinate

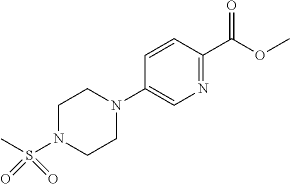

Compound methyl 5-(4-(methylsulfonyl)piperazin-1-yl)picolinate was prepared from methyl 5-(piperazin-1-yl)picolinate hydrochloride (8.00 g, 31.04 mmol) and methanesulfonyl chloride (5.33 g, 46.53 mmol), following a procedure similar to that described for the synthesis of methyl 3-(4-(methylsulfonyl)piperazin-1-yl)bicyclo[1.1.1]pentane-1-carboxylate, and was isolated as an off-white solid.

Yield 3.73 g (40%). ¹H NMR (400 MHz, DMSO) δ 8.42 (d, J=2.8 Hz, 1H), 7.91 (d, J=8.8 Hz, 1H), 7.41 (dd, J=2.8, 8.8 Hz, 1H), 3.82 (s, 3H), 3.55-3.47 (m, 4H), 3.28-3.22 (m, 4H), 2.93 (s, 3H). m/z: [ESI⁺] 300 (M+H)⁺.

### Synthesis of 5-(4-(methylsulfonyl)piperazin-1-yl)picolinic acid

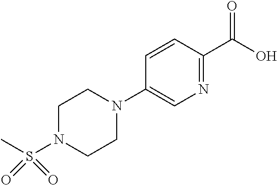 [SMILES: CS(=O)(=O)N1CCN(c2ccc(C(=O)O)nc2)CC1]

Compound 5-(4-(methylsulfonyl)piperazin-1-yl)picolinic acid was prepared from methyl 5-(4-(methylsulfonyl)piperazin-1-yl)picolinate (1.80 g, 6.01 mmol) and lithium hydroxide monohydrate (1.01 g, 24.07 mmol), following a procedure similar to that described for the synthesis of 4-(1,1-dioxidotetrahydro-2H-thiopyran-4-yl)benzoic acid, and was isolated as an off-white solid.

Yield 1.58 g (92%). ¹H NMR (400 MHz, DMSO) δ 8.40 (d, J=2.8 Hz, 1H), 7.91 (d, J=8.8 Hz, 1H), 7.42 (dd, J=2.8, 8.8 Hz, 1H), 3.51 (t, J=4.8 Hz, 4H), 3.26 (t, J=4.8 Hz, 4H), 2.93 (s, 3H). Carboxylic acid proton not observed. m/z: [ESI⁺] 286 (M+H)⁺.

### Synthesis of 5-(4-(tert-butoxycarbonyl)piperazin-1-yl)picolinic acid

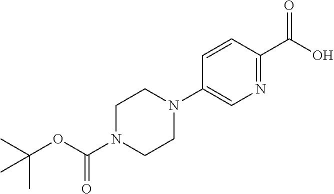 [SMILES: CC(C)(C)OC(=O)N1CCN(c2ccc(C(=O)O)nc2)CC1]

Compound 5-(4-(tert-butoxycarbonyl)piperazin-1-yl)picolinic acid was prepared from ten-butyl 4-(6-(methoxycarbonyl)pyridin-3-yl)piperazine-1-carboxylate (5.00 g, 15.56 mmol) and lithium hydroxide monohydrate (2.61 g, 62.20 mmol), following a procedure similar to that described for the synthesis of 4-(1,1-dioxidotetrahydro-2H-thiopyran-4-yl)benzoic acid, and was isolated as an off-white solid.

Yield 3.07 g (64%). ¹H NMR (400 MHz, DMSO) δ 12.51 (br s, 1H), 8.36 (d, J=2.8 Hz, 1H), 7.87 (d, J=8.8 Hz, 1H), 7.36 (dd, J=2.8, 8.8 Hz, 1H), 3.51-3.43 (m, 4H), 3.39-3.32 (m, 4H), 1.42 (s, 9H). m/z: [ESI⁺] 308 (M+H)⁺.

### Synthesis of tert-butyl 4-(6-((4-(2-chlorophenyl)thiazol-2-yl)carbamoyl)pyridin-3-yl)piperazine-1-carboxylate (Method 1)

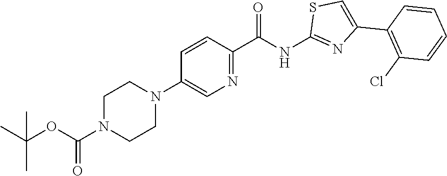 [SMILES: CC(C)(C)OC(=O)N1CCN(c2ccc(C(=O)Nc3nc(-c4ccccc4Cl)cs3)nc2)CC1]

To a stirred solution of 5-(4-(tert-butoxycarbonyl)piperazin-1-yl)picolinic acid (1.00 g, 3.25 mmol) in DMF (5 mL) was added CDI (0.79 g, 4.87 mmol) at room temperature under a nitrogen atmosphere. The resulting mixture was stirred for 2 h at 50° C. under a nitrogen atmosphere to form solution A. Concurrently, to a stirred solution of 4-(2-chlorophenyl)thiazol-2-amine (0.75 g, 3.56 mmol) in DMF (5 mL) was added sodium hydride (60% dispersion in mineral oil, 0.38 g, 9.50 mmol), at room temperature under a nitrogen atmosphere. The resulting mixture was stirred for 30 min at room temperature to form solution B. Subsequently, solution B was added dropwise to solution A at room temperature under a nitrogen atmosphere. The resulting solution was stirred for 16 h at room temperature under a nitrogen atmosphere. The resulting mixture was diluted with water (50 mL) and extracted with ethyl acetate (3×50 mL). The combined organic layers were dried over anhydrous Na₂SO₄. After filtration, the filtrate was concentrated under reduced pressure. The residue was purified by reverse phase flash chromatography with the following conditions: Column: WelFlash TM C18-I, 20-40 μm, 330 g; Eluent A: water (plus 10 mmol/L NH₄HCO₃); Eluent B: acetonitrile; Gradient: 50%70% B in 25 min; Flow rate: 80 mL/min; Detector: UV 220/254 nm. The desired fractions were collected and concentrated under reduced pressure to afford tert-butyl 4-(6-((4-(2-chlorophenyl)thiazol-2-yl)carbamoyl)pyridin-3-yl)piperazine-1-carboxylate as a yellow solid.

Yield 1.39 g (85%). ¹H NMR (400 MHz, DMSO) δ 11.65 (br s, 1H), 8.42 (d, J=2.8 Hz, 1H), 8.02 (d, J=8.8 Hz, 1H), 7.93 (dd, J=2.0, 7.6 Hz, 1H), 7.71 (s, 1H), 7.57 (dd, J=1.6, 7.6 Hz, 1H), 7.49 (dd, J=2.8, 8.8 Hz, 1H), 7.47-7.35 (m, 2H), 3.54-3.47 (m, 4H), 3.47-3.40 (m, 4H), 1.44 (s, 9H). m/z: [ESI⁺] 500, 502 (M+H)⁺.

### Synthesis of N-(4-(2-chlorophenyl)thiazol-2-yl)-5-(piperazin-1-yl)picolinamide hydrochloride

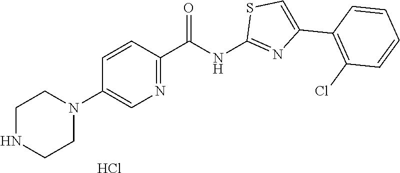 [SMILES: O=C(Nc1nc(-c2ccccc2Cl)cs1)c1ccc(N2CCNCC2)cn1 ; Cl]

Compound N-(4-(2-chlorophenyl)thiazol-2-yl)-5-(piperazin-1-yl)picolinamide hydrochloride was prepared from tert-butyl 4-(6-((4-(2-chlorophenyl)thiazol-2-yl)carbamoyl)pyridin-3-yl)piperazine-1-carboxylate (20.00 g, 40.00 mmol) following a procedure similar to that described for the synthesis of methyl 5-(piperazin-1-yl)picolinate hydrochloride and was isolated as a yellow solid.

Yield 16.00 g (92%). ¹H NMR (400 MHz, DMSO) δ 11.83 (br s, 1H), 9.58 (br s, 2H, NH·HCl), 8.48 (d, J=2.8 Hz, 1H), 8.09 (d, J=8.8 Hz, 1H), 7.92 (dd, J=2.0, 7.6 Hz, 1H), 7.73 (s, 1H), 7.61 (dd, J=2.8, 8.8 Hz, 1H), 7.58 (dd, J=1.6, 7.6 Hz, 1H), 7.49-7.36 (m, 2H), 3.78-3.63 (m, 4H), 3.30-3.17 (m, 4H). m/z: [ESI⁺]400, 402 (M+H)⁺.

### Synthesis of N-(4-(2-chlorophefnyl)thiazo2-yl)-5-fluoropicolinamide

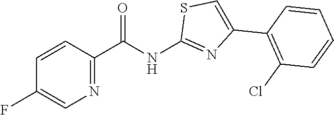 [SMILES: O=C(Nc1nc(-c2ccccc2Cl)cs1)c1ccc(F)cn1]

To a stirred mixture of 5-fluoropicolinic acid (1.00 g, 7.09 mmol) and triethylamine (2.15 g, 21.25 mmol) in ethyl acetate (20 mL) were added 4-(2-chlorophenyl)thiazol-2-amine (1.94 g, 9.21 mmol) and T3P (50% wt. in ethyl acetate, 13.52 g, 21.25 mmol) portionwise at room temperature, under a nitrogen atmosphere. The resulting mixture was stirred overnight at 70° C. under a nitrogen atmosphere. The resulting mixture was cooled down to room temperature. The precipitated solid was collected by filtration and washed with ethyl acetate (5×5 mL) to afford N-(4-(2-chlorophenyl)thiazol-2-yl)-5-fluoropicolinamide as an off-white solid.

Yield 1.50 g (63%). ¹H NMR (400 MHz, CDCl₃) δ 11.08 (br s, 1H), 8.53 (d, J=2.8 Hz, 1H), 8.38 (dd, J=4.4, 8.8 Hz, 1H), 7.93 (dd, J=1.6, 7.6 Hz, 1H), 7.72-7.61 (m, 1H), 7.59 (s, 1H), 7.50 (dd, J=1.6, 8.0 Hz, 1H), 7.40-7.35 (m, 1H), 7.32-7.27 (m, 1H). ¹⁹F NMR (376 MHz, CDCl₃) δ 119.38 m/z: [ESI⁺] 334, 336 (M+H)⁺.

### Synthesis of tert-butyl 4-(6-((4-(2-chlorophenyl)thiazol-2-yl)carbamoyl)pyridin-3-yl)piperazine-1-carboxylate (Method 2)

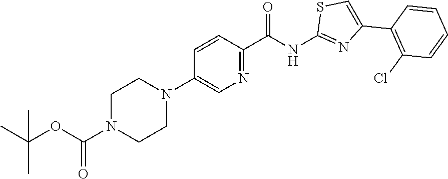 [SMILES: CC(C)(C)OC(=O)N1CCN(c2ccc(C(=O)Nc3nc(-c4ccccc4Cl)cs3)nc2)CC1]

Compound tert-butyl 4-(6-((4-(2-chlorophenyl)thiazol-2-yl)carbamoyl)pyridin-3-yl)piperazine-1-carboxylate was prepared from N-(4-(2-chlorophenyl)thiazol-2-yl)-5-fluoropicolinamide (22.00 g, 65.91 mmol) and tert-butyl piperazine-1-carboxylate (18.42 g, 98.89 mmol) following a procedure similar to that described for the synthesis of 2-chloro-4-morpholinobenzaldehyde and was isolated as a dark yellow solid.

Yield 32.00 g (97%). ¹H NMR (400 MHz, CDCl₃) δ 11.11 (br s, 1H), 8.27 (d, J=2.8 Hz, 1H), 8.17 (d, J=8.8 Hz, 1H), 7.93 (dd, J=1.6, 7.6 Hz, 1H), 7.53 (s, 1H), 7.50 (dd, J=1.6, 8.0 Hz, 1H), 7.40-7.35 (m, 1H), 7.32-7.25 (m, 2H), 3.65 (t, J=5.2 Hz, 4H), 3.41 (t, J=5.2 Hz, 4H), 1.52 (s, 9H). m/z: [ESI⁺] 500, 502 (M+H)⁺.

### Synthesis of tert-butyl 6-(6-((4-(2-chlorophenyl)thiazol-2-yl)carbamoyl)pyridin-3-yl)-2,6-diazaspiro[3.3]heptane-2-carboxylate

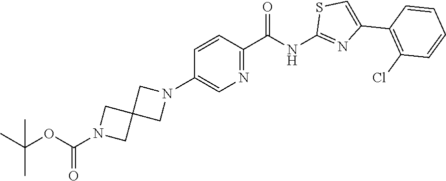 [SMILES: CC(C)(C)OC(=O)N1CC2(C1)CN(c1ccc(C(=O)Nc3nc(-c4ccccc4Cl)cs3)nc1)C2]

Compound tert-butyl 6-(6-((4-(2-chlorophenyl)thiazol-2-yl)carbamoyl)pyridin-3-yl)-2,6-diazaspiro[3.3]heptane-2-carboxylate was prepared from N-(4-(2-chlorophenyl)thiazol-2-yl)-5-fluoropicolinamide (1.00 g, 3.00 mmol) and tert-butyl 2,6-diazaspiro[3.3]heptane-2-carboxylate (0.71 g, 3.58 mmol), following a procedure similar to that described for the synthesis of 2-chloro-4-morpholinobenzaldehyde and was isolated as an orange liquid.

Yield 1.30 g (85%). ¹H NMR (400 MHz, CDCl₃) δ 11.02 (br s, 1H), 8.09 (d, J=8.4 Hz, 1H), 7.91 (dd, J=1.6, 7.6 Hz, 1H), 7.77 (d, J=2.8 Hz, 1H), 7.50 (s, 1H), 7.47 (dd, J=1.6, 8.0 Hz, 1H), 7.38-7.31 (m, 1H), 7.29-7.23 (m, 1H), 6.79 (dd, J=2.8, 8.4 Hz, 1H), 4.18 (s, 4H), 4.15 (s, 4H), 1.46 (s, 9H). m/z: [ESI⁺] 512, 514 (M+H)⁺.

### Synthesis of N-(4-(2-chlorophenyl)thiazol-2-yl)-5-(2,6-diazaspiro[3.3]heptan-2-yl)picolinamide 2,2,2-trifluoroacetate salt

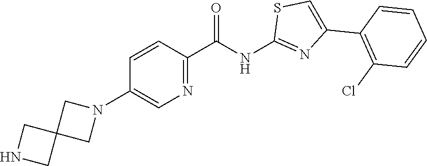 [SMILES: O=C(Nc1nc(-c2ccccc2Cl)cs1)c1ccc(N2CC3(CNC3)C2)cn1]

A mixture of tert-butyl 6-(6-((4-(2-chlorophenyl)thiazol-2-yl)carbamoyl)pyridin-3-yl)-2,6-diazaspiro[3.3]heptane-2-carboxylate (1.30 g, 2.54 mmol) and 2,2,2-trifluoroacetic acid (13 mL) in DCM (13 mL) was stirred overnight at room temperature under a nitrogen atmosphere. The resulting mixture was concentrated under reduced pressure to afford N-(4-(2-chlorophenyl)thiazol-2-yl)-5-(2,6-diazaspiro[3.3]heptan-2-yl)picolinamide 2,2,2-trifluoroacetic acid salt as a yellow liquid.

Yield 1.30 g (97%). ¹H NMR (400 MHz, DMSO) δ 11.57 (br s, 1H), 8.00 (d, J=8.8 Hz, 1H), 7.95-7.88 (m, 2H), 7.70 (s, 1H), 7.56 (dd, J=1.6, 8.0 Hz, 1H), 7.49-7.35 (m, 2H), 7.01 (dd, J=2.8, 8.8 Hz, 1H), 4.24-4.19 (m, 8H). Aliphatic NH proton not observed. m/z: [ESI⁺] 412, 414 (M+H)⁺.

### Synthesis of tert-butyl 7-(6-(methoxycarbonyl)pyridin-3-yl)-2,7-diazaspiro[3.5]nonane-2-carboxylate

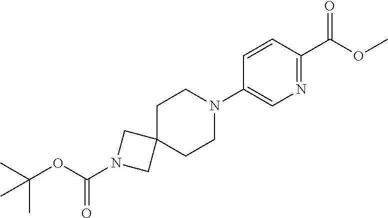 [SMILES: COC(=O)c1ccc(N2CCC3(CC2)CN(C(=O)OC(C)(C)C)C3)cn1]

Compound tert-butyl 7-(6-(methoxycarbonyl)pyridin-3-yl)-2,7-diazaspiro[3.5]nonane-2-carboxylate was prepared from methyl 5-bromopicolinate (1.60 g, 7.41 mmol) and tert-butyl 2,7-diazaspiro[3.5]nonane-2-carboxylate (1.40 g, 6.19 mmol) following a procedure similar to that described for the synthesis of methyl 5-(2-methyl-1-oxo-2,8-diazaspiro[4.5]decan-8-yl)picolinate and was isolated as a yellow solid.

Yield 2.20 g (98%). ¹H NMR (400 MHz, CDCl₃) δ 8.35 (d, J=2.8 Hz, 1H), 7.99 (d, J=8.8 Hz, 1H), 7.16 (dd, J=2.8, 8.8 Hz, 1H), 3.96 (s, 3H), 3.71 (s, 4H), 3.38-3.29 (m, 4H), 1.94-1.84 (m, 4H), 1.46 (s, 9H). m/z: [ESI⁺] 362 (M+H)⁺.

### Synthesis of 5-(2-(tert-butoxycarbonyl)-2,7-diazaspiro[3.5]nonan-7-yl)picolinic acid

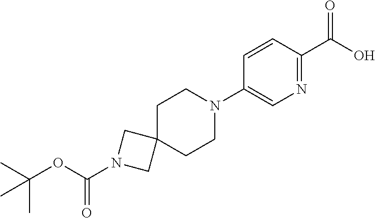 [SMILES: CC(C)(C)OC(=O)N1CC2(CCN(c3ccc(C(=O)O)nc3)CC2)C1]

Compound 5-(2-(tert-butoxycarbonyl)-2,7-diazaspiro[3.5]nonan-7-yl)picolinic acid was prepared from tert-butyl 7-(6-(methoxycarbonyl)pyridin-3-yl)-2,7-diazaspiro[3.5]nonane-2-carboxylate (2.00 g, 5.53 mmol) and lithium hydroxide monohydrate (928 mg, 22.12 mmol), following a procedure similar to that described for the synthesis of 4-(1,1-dioxidotetrahydro-2H-thiopyran-4-yl)benzoic acid and was isolated as an off-white solid.

Yield 1.56 g (81%). ¹H NMR (400 MHz, CDCl₃) (8.28 (d, J=2.8 Hz, 1H), 8.04 (d, J=8.8 Hz, 1H), 7.24 (dd, J=2.8, 8.8 Hz, 1H), 3.71 (s, 4H), 3.43-3.25 (m, 4H), 1.94-1.79 (m, 4H), 1.47 (s, 9H). Carboxylic acid proton not observed. m/z: [ESI⁺] 348 (M+H)⁺.

### Synthesis of tert-butyl 7-(6-((4-(2-chlorophenyl)thiazol-2-yl)carbamoyl)pyridin-3-yl)-2,7-diazaspiro[3.5]nonane-2-carboxylate

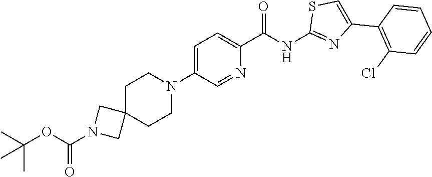 [SMILES: CC(C)(C)OC(=O)N1CC2(CCN(c3ccc(C(=O)Nc4nc(-c5ccccc5Cl)cs4)nc3)CC2)C1]

Compound tert-butyl 7-(6-((4-(2-chlorophenyl)thiazol-2-yl)carbamoyl)pyridin-3-yl)-2,7-diazaspiro[3.5]nonane-2-carboxylate was prepared from 5-(2-(tert-butoxycarbonyl)-2,7-diazaspiro[3.5]nonan-7-yl)picolinic acid (1.40 g, 4.03 mmol) and 4-(2-chlorophenyl)thiazol-2-amine (0.85 g, 4.03 mmol) following a procedure similar to that described for the synthesis of tert-butyl 4-(6-((4-(2-chlorophenyl)thiazol-2-yl)carbamoyl)pyridin-3-yl)piperazine-1-carboxylate and was isolated as a yellow solid.

Yield 0.93 g (43%). ¹H NMR (400 MHz, CDCl₃) δ 11.04 (br s, 1H), 8.26 (d, J=2.8 Hz, 1H), 8.13 (d, J=8.8 Hz, 1H), 7.93 (dd, J=1.6, 7.6 Hz, 1H), 7.53 (s, 1H), 7.51-7.47 (m, 1H), 7.39-7.33 (m, 1H), 7.31-7.24 (m, 2H), 3.73 (s, 4H), 3.43-3.33 (m, 4H), 1.96-1.87 (m, 4H), 1.48 (s, 9H). m/z: [ESI⁺] 540, 542 (M+H)⁺.

### Synthesis of N-(4-(2-chlorophenyl)thiazol-2-yl)-5-(2,7-diazaspiro[3.5]nonan-7-yl)picolinamide hydrochloride

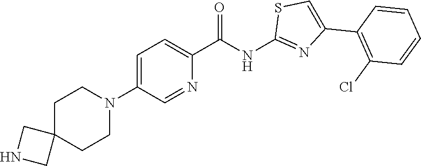 [SMILES: O=C(Nc1nc(-c2ccccc2Cl)cs1)c1ccc(N2CCC3(CC2)CNC3)cn1]

Compound N-(4-(2-chlorophenyl)thiazol-2-yl)-5-(2,7-diazaspiro[3.5]nonan-7-yl)picolinamide hydrochloride was prepared from tert-butyl 7-(6-((4-(2-chlorophenyl)thiazol-2-yl)carbamoyl)pyridin-3-yl)-2,7-diazaspiro[3.5]nonane-2-carboxylate (900 mg, 1.67 mmol) following a procedure similar to that described for the synthesis of methyl 5-(piperazin-1-yl)picolinate hydrochloride and was isolated as a black solid.

Yield 682 mg (86%). ¹H NMR (400 MHz, DMSO) δ 9.68 (br s, 2H, NH·HCl), 9.40 (br s, 1H), 8.43 (d, J=2.8 Hz, 1H), 8.06 (d, J=8.8 Hz, 1H), 7.91 (dd, J=1.6, 7.6 Hz, 1H), 7.71 (s, 1H), 7.60-7.53 (m, 2H), 7.48-7.35 (m, 3H), 3.75 (s, 4H), 3.49-3.41 (m, 4H), 1.89 (t, J=5.6 Hz, 4H). m/z: [ESI⁺] 440, 442 (M+H)⁺.

### Synthesis of methyl 5-(4-((tert-butyldimethylsilyl)oxy)piperidin-1-yl)picolinate

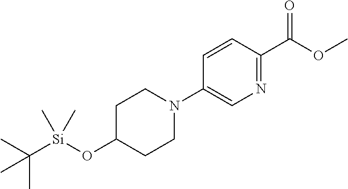 [SMILES: COC(=O)c1ccc(N2CCC(O[Si](C)(C)C(C)(C)C)CC2)cn1]

Compound methyl 5-(4-((tert-butyldimethylsilyl)oxy)piperidin-1-yl)picolinate was prepared from methyl 5-bromopicolinate (3.80 g, 17.59 mmol) and 4-((tert-butyldimethylsilyl)oxy)piperidine (5.68 g, 26.37 mmol) following a procedure similar to that described for the synthesis of methyl 5-(2-methyl-1-oxo-2,8-diazaspiro[4.5]decan-8-yl)picolinate and was isolated as an off-white solid.

Yield 3.50 g (57%). ¹H NMR (400 MHz, CDCl₃) δ 8.35 (d, J=2.8 Hz, 1H), 7.99 (d, J=8.8 Hz, 1H), 7.15 (dd, J=2.8, 8.8 Hz, 1H), 4.02-3.97 (m, 1H), 3.97 (s, 3H), 3.66-3.54 (m, 2H), 3.39-3.25 (m, 2H), 1.95-1.80 (m, 2H), 1.75-1.60 (m, 2H), 0.91 (s, 9H), 0.09 (s, 6H). m/z: [ESI⁺] 351 (M+H)⁺.

### Synthesis of 5-(4-((tert-butyldimethylsilyl)oxy)piperidin-1-yl)picolinic acid

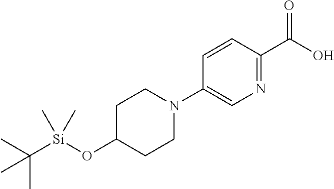 [SMILES: CC(C)(C)[Si](C)(C)OC1CCN(c2ccc(C(=O)O)nc2)CC1]

Compound 5-(4-((tert-butyldimethylsilyl)oxy)piperidin-1-yl)picolinic acid was prepared from methyl 5-(4-((tert-butyldimethylsilyl)oxy)piperidin-1-yl)picolinate (1.70 g, 4.85 mmol) and lithium hydroxide monohydrate (814 mg, 19.40 mmol), following a procedure similar to that described for the synthesis of 4-(1,1-dioxidotetrahydro-2H-thiopyran-4-yl)benzoic acid and was isolated as an off-white solid.

Yield 1.50 g (92%). ¹H NMR (400 MHz, CDCl₃) δ 8.22 (d, J=2.8 Hz, 1H), 8.05 (d, J=8.8 Hz, 1H), 7.25 (dd, J=2.8, 8.8 Hz, 1H), 4.08-3.97 (m, 1H), 3.73-3.55 (m, 2H), 3.42-3.27 (m, 2H), 1.95-1.81 (m, 2H), 1.76-1.62 (m, 2H), 0.93 (s, 9H), 0.11 (s, 6H). Carboxylic acid proton not observed. m/z: [ESI⁺] 337 (M+H)⁺.

### Synthesis of N-(4-bromothiazol-2-yl)-5-(4-((tert-butyldimethylsilyl)oxy)piperidin-1-yl)picolinamide

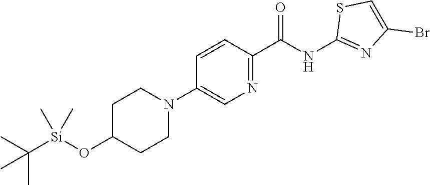 [SMILES: CC(C)(C)[Si](C)(C)OC1CCN(c2ccc(C(=O)Nc3nc(Br)cs3)nc2)CC1]

Compound N-(4-bromothiazol-2-yl)-5-(4-((tert-butyldimethylsilyl)oxy)piperidin-1-yl)picolinamide was prepared from 5-(4-((tert-butyldimethylsilyl)oxy)piperidin-1-yl)picolinic acid (2.00 g, 5.94 mmol) and 4-bromothiazol-2-amine (1.38 g, 7.71 mmol), following the procedure similar to that described for the synthesis of N-(4-(2-chlorophenyl)thiazol-2-yl)-5-fluoropicolinamide and was isolated as a yellow solid.

Yield 2.10 g (71%). ¹H NMR (400 MHz, CDCl₃) δ 11.02 (br s, 1H), 8.23 (d, J=2.8 Hz, 1H), 8.07 (d, J=8.8 Hz, 1H), 7.23 (dd, J=2.8, 8.8 Hz, 1H), 6.89 (s, 1H), 4.08-3.95 (m, 1H), 3.69-3.55 (m, 2H), 3.41-3.27 (m, 2H), 1.97-1.81 (m, 2H), 1.77-1.62 (m, 2H), 0.93 (s, 9H), 0.11 (s, 6H). m/z: [ESI⁺] 497, 499 (M+H)⁺.

### Synthesis of 5-(4-((tert-butyldimethylsilyl)oxy)piperidin-1-yl)-N-(4-(2-(methoxymethyl)phenyl) thiazol-2-yl)picolinamide

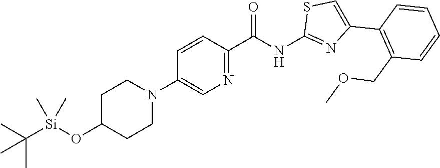 [SMILES: COCc1ccccc1-c1csc(NC(=O)c2ccc(N3CCC(O[Si](C)(C)C(C)(C)C)CC3)cn2)n1]

Compound 5-(4-((tert-butyldimethylsilyl)oxy)piperidin-1-yl)-N-(4-(2-(methoxymethyl)phenyl)thiazol-2-yl)picolinamide was prepared from N-(4-bromothiazol-2-yl)-5-(4-((tert-butyldimethylsilyl)oxy)piperidin-1-yl)picolinamide (0.40 g, 0.81 mmol) and (2-(methoxymethyl)phenyl)boronic acid (0.27 g, 1.63 mmol) following a procedure similar to that described for the synthesis of methyl 4-(3,6-dihydro-2H-thiopyran-4-yl)benzoate and was isolated as a light yellow solid.

Yield 0.10 g (23%). ¹H NMR (400 MHz, CDCl₃) δ 511.03 (br s, 1H), 8.27 (d, J=2.8 Hz, 1H), 8.13 (d, J=8.8 Hz, 1H), 7.81-7.67 (m, 1H), 7.61-7.50 (m, 1H), 7.42-7.37 (m, 2H), 7.26 (dd, J=2.8, 8.8 Hz, 1H), 7.20 (s, 1H), 4.61 (s, 2H), 4.07-3.95 (m, 1H), 3.72-3.56 (m, 2H), 3.45 (s, 3H), 3.40-3.27 (m, 2H), 1.96-1.83 (m, 2H), 1.77-1.63 (m, 2H), 0.93 (s, 9H), 0.11 (s, 6H). m/z: [ESI⁺] 539 (M+H)⁺.

### Synthesis of tert-butyl (4-(2-oxopyrrolidin-1-yl)thiazol-2-yl)carbamate

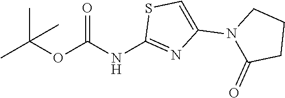 [SMILES: CC(C)(C)OC(=O)Nc1nc(N2CCCC2=O)cs1]

Compound tert-butyl (4-(2-oxopyrrolidin-1-yl)thiazol-2-yl)carbamate was prepared from tert-butyl (4-bromothiazol-2-yl)carbamate (1.00 g, 3.58 mmol) and pyrrolidin-2-one (0.46 g, 5.40 mmol) following a procedure similar to that described for the synthesis of methyl 5-(2-methyl-1-oxo-2,8-diazaspiro[4.5]decan-8-yl)picolinate and was isolated as a yellow solid.

Yield 450 mg (44%). ¹H NMR (400 MHz, CDCl₃) δ 8.03 (br s, 111), 7.34 (s, 1H), 4.03-3.93 (m, 2H), 2.66-2.56 (m, 2H), 2.22-2.06 (m, 2H), 1.56 (s, 9H). m/z: [ESI⁺] 284 (M+H)⁺.

### Synthesis of 1-(2-aminothiazol-4-yl)pyrrolidin-2-one

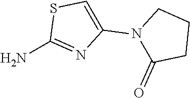 [SMILES: Nc1nc(N2CCCC2=O)cs1]

Compound 1-(2-aminothiazol-4-yl)pyrrolidin-2-one was prepared from tert-butyl (4-(2-oxopyrrolidin-1-yl)thiazol-2-yl)carbamate (400 mg, 1.41 mmol) following the procedure similar to that described for the synthesis of N-(4-(2-chlorophenyl)thiazol-2-yl)-5-(2,6-diazaspiro[3.3]heptan-2-yl)picolinamide 2,2,2-trifluoroacetic acid salt, except that purification was achieved by reverse phase flash chromatography with the following conditions: column, C18 silica gel; Eluent A: water (plus 10 mmol/L NH₄HCO₃); Eluent B: MeOH; Gradient: 30%-60% B in 30 min; Flow rate: 80 mL/min; Detector: UV 220/254 nm; and was isolated as an off-white solid.

Yield 200 mg (77%). ¹H NMR (400 MHz, DMSO) δ 7.06 (br s, 2H), 6.63 (s, 1H), 3.85 (t, J=7.2 Hz, 2H), 2.42 (t, J=8.0 Hz, 2H), 2.06-1.92 (m, 2H). m/z: [ESI⁺] 184 (M+H)⁺.

### Synthesis of methyl 5-((1-(tert-butoxycarbonyl)piperidin-4-yl)oxy)picolinate

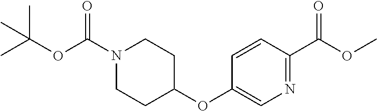 [SMILES: COC(=O)c1ccc(OC2CCN(C(=O)OC(C)(C)C)CC2)cn1]

Compound methyl 5-((1-(tert-butoxycarbonyl)piperidin-4-yl)oxy)picolinate was prepared from methyl 5-hydroxypicolinate (153 mg, 1.00 mmol) and tert-butyl 4-hydroxypiperidine-1-carboxylate (403 mg, 2.00 mmol), following a procedure similar to that described for the synthesis of methyl 5-((1-methylpiperidin-4-yl)oxy)picolinate and was isolated as a yellow solid.

Yield 110 mg (33%). ¹H NMR (400 MHz, DMSO) δ 8.37 (d, J=2.8 Hz, 1H), 7.99 (d, J=8.8 Hz, 1H), 7.56 (dd, J=2.8, 8.8 Hz, 1H), 4.83-4.70 (m, 1H), 3.80 (s, 3H), 3.74-3.61 (m, 2H), 3.26-3.11 (m, 2H), 2.01-1.87 (m, 2H), 1.61-1.47 (m, 2H), 1.41 (s, 9H). m/z: [ESI⁺] 337 (M+H)⁺.

### Synthesis of 5-((1-(tert-butoxycarbonyl)piperidin-4-yl)oxy)picolinic acid

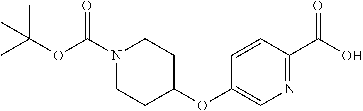 [SMILES: CC(C)(C)OC(=O)N1CCC(Oc2ccc(C(=O)O)nc2)CC1]

Compound 5-((1-(tert-butoxycarbonyl)piperidin-4-yl)oxy)picolinic acid was prepared from methyl 5-((1-(tert-butoxycarbonyl)piperidin-4-yl)oxy)picolinate (100 mg, 0.30 mmol) and lithium hydroxide monohydrate (50 mg, 1.19 mmol) following a procedure similar to that described for the synthesis of 4-(1,1-dioxidotetrahydro-2H-thiopyran-4-yl)benzoic acid and was isolated as an off-white solid.

Yield 79 mg (82%). ¹H NMR (400 MHz, DMSO) δ 8.36 (d, J=2.8 Hz, 1H), 8.00 (d, J=8.8 Hz, 1H), 7.56 (dd, J=2.8, 8.8 Hz, 1H), 4.82-4.69 (m, 1H), 3.74-3.60 (m, 2H), 3.26-3.11 (m, 2H), 2.01-1.87 (m, 2H), 1.61-1.47 (m, 2H), 1.41 (s, 9H). Carboxylic acid proton not observed. m/z: [ESI⁺] 323 (M+H)⁺.

### Synthesis of tert-butyl 4-((6-((4-(2-chlorophenyl)thiazol-2-yl)carbamoyl)pyridin-3-yl)oxy)piperidine-1-carboxylate

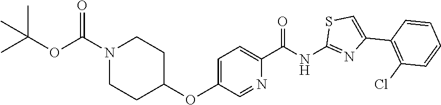 [SMILES: CC(C)(C)OC(=O)N1CCC(Oc2ccc(C(=O)Nc3nc(-c4ccccc4Cl)cs3)nc2)CC1]

Compound tert-butyl 4-((6-((4-(2-chlorophenyl)thiazol-2-yl)carbamoyl)pyridin-3-yl)oxy)piperidine-1-carboxylate was prepared from 5-((1-(tert-butoxycarbonyl)piperidin-4-yl)oxy)picolinic acid (0.70 g, 2.17 mmol) and 4-(2-chlorophenyl)thiazol-2-amine (0.46 g, 2.18 mmol) following a procedure similar to that described for the synthesis of tert-butyl 4-(6-((4-(2-chlorophenyl)thiazol-2-yl)carbamoyl)pyridin-3-yl)piperazine-1-carboxylate and was isolated as a yellow solid.

Yield 0.93 g (83%). ¹H NMR (400 MHz, CDCl₃) δ 11.04 (br s, 1H), 8.26 (d, J=2.8 Hz, 1H), 8.13 (d, J=8.8 Hz, 1H), 7.93 (dd, J=1.6, 7.6 Hz, 1H), 7.53 (s, 1H), 7.51-7.47 (m, 1H), 7.39-7.33 (m, 1H), 7.31-7.24 (m, 2H), 3.65-3.78 (m, 1H), 3.43-3.33 (m, 4H), 1.96-1.87 (m, 4H), 1.48 (s, 9H). m/z: [ESI⁺] 515, 517 (M+H)⁺.

### Synthesis of N-(4-(2-chlorophenyl)thiazol-2-yl)-5-(piperidin-4-yloxy)picolinamide hydrochloride

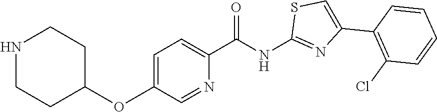 [SMILES: O=C(Nc1nc(-c2ccccc2Cl)cs1)c1ccc(OC2CCNCC2)cn1]

Compound N-(4-(2-chlorophenyl)thiazol-2-yl)-5-(piperidin-4-yloxy)picolinamide hydrochloride was prepared from tert-butyl 4-((6-((4-(2-chlorophenyl)thiazol-2-yl)carbamoyl)pyridin-3-yl)oxy)piperidine-1-carboxylate (270 mg, 0.52 mmol) following a procedure similar to that described for the synthesis of methyl 5-(piperazin-1-yl)picolinate hydrochloride and was isolated as an off-white solid.

Yield 100 mg (42%). ¹H NMR (400 MHz, DMSO) δ 11.94 (br s, 1H), 8.85 (br s, 2H, NH·HCl), 8.49 (d, J=2.8 Hz, 1H), 8.19 (d, J=8.8 Hz, 1H), 7.92 (dd, J=2.0, 7.6 Hz, 1H), 7.78-7.73 (m, 1H), 7.58 (dd, J=1.6, 7.6 Hz, 1H), 7.49-7.36 (m, 3H), 4.99-4.87 (m, 1H), 3.33-3.23 (m, 2H), 3.16-3.02 (m, 2H), 2.23-2.09 (m, 2H), 2.00-1.83 (m, 2H). m/z: [ESI⁺] 415, 417 (M+H)⁺.

### Synthesis of tert-butyl (4-(3,6-dihydro-2H-pyran-4-yl)thiazol-2-yl)carbamate

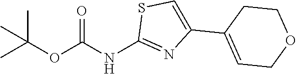 [SMILES: CC(C)(C)OC(=O)Nc1nc(C2=CCOCC2)cs1]

Compound tert-butyl (4-(3,6-dihydro-2H-pyran-4-yl)thiazol-2-yl)carbamate was prepared from tert-butyl (4-bromothiazol-2-yl)carbamate (2.00 g, 7.16 mmol) and 2-(3,6-dihydro-2H-pyran-4-yl)-4,4,5,5-tetramethyl-1,3,2-dioxaborolane (3.01 g, 14.33 mmol) following a procedure similar to that described for the synthesis of methyl 4-(3,6-dihydro-2H-thiopyran-4-yl)benzoate and was isolated as an off-white solid.

Yield 760 mg (38%). ¹H NMR (400 MHz, DMSO) δ 11.45 (br s, 1H), 6.97 (s, 1H), 6.45 (s, 1H), 4.21 (q, J=2.8 Hz, 2H), 3.78 (t, J=5.6 Hz, 2H), 2.41-2.32 (m, 2H), 1.48 (s, 911). m/z: [ESI⁺] 283 (M+H)⁺.

### Synthesis of tert-butyl (4-(tetrahydro-2H-pyran-4-yl)thiazol-2-yl)carbamate

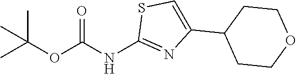 [SMILES: CC(C)(C)OC(=O)Nc1nc(C2CCOCC2)cs1]

Compound tert-butyl (4-(tetrahydro-2H-pyran-4-yl)thiazol-2-yl)carbamate was prepared from tert-butyl (4-(3,6-dihydro-2H-pyran-4-yl)thiazol-2-yl)carbamate (700 mg, 2.48 mmol) following a procedure similar to that described for the synthesis of methyl 4-(tetrahydro-2H-thiopyran-4-yl)benzoate and was isolated as an off-white solid.

Yield 680 mg (96%). ¹H NMR (400 MHz, DMSO) δ 11.34 (br s, 1H), 6.72 (s, 1H), 3.97-3.88 (m, 2H), 3.39 (dt, J=2.0, 11.6 Hz, 2H), 2.80-2.75 (m, 1H), 1.84-1.76 (m, 2H), 1.64-1.55 (m, 2H), 1.46 (s, 9H). m/z: [ESI⁺]285 (M+H)⁺.

### Synthesis of 4-(tetrahydro-2H-pyran-4-yl)thiazol-2-amine

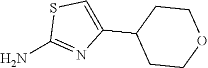 [SMILES: Nc1nc(C2CCOCC2)cs1]

Compound 4-(tetrahydro-2H-pyran-4-yl)thiazol-2-amine was prepared from tert-butyl (4-(tetrahydro-2H-pyran-4-yl)thiazol-2-yl)carbamate (700 mg, 2.46 mmol) following the procedure similar to that described for the synthesis of N-(4-(2-chlorophenyl)thiazol-2-yl)-5-(2,6-diazaspiro[3.3]heptan-2-yl)picolinamide 2,2,2-trifluoroacetic acid salt, with the exception that it was purified by reverse phase flash chromatography with the following conditions: Column: WelFlash TM C18-I, 20-40 μm, 120 g; Eluent A: water (plus 10 mmol/L NH₄HCO₃); Eluent B: acetonitrile; Gradient: 35%-55% B in 20 min; Flow rate: 60 m/min; Detector: UV 220/254 nm; and was isolated as an off-white solid.

Yield 320 mg (71%). ¹H NMR (400 MHz, DMSO) δ 6.82 (br s, 2H), 6.11 (s, 1H), 3.92-3.82 (m, 2H), 3.40-3.34 (m, 2H), 2.65-2.53 (m, 1H), 1.80-1.70 (m, 2H), 1.62-1.46 (m, 2H). m/z: [ESI⁺] 185 (M+H)⁺.

### Synthesis of N-(4-(2-chlorophenyl)-1H-imidazol-2-yl)acetamide

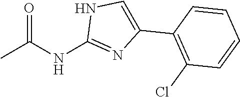 [SMILES: CC(=O)Nc1nc(-c2ccccc2Cl)c[nH]1]

To a stirred solution of N-carbamimidoylacetamide (1.30 g, 12.86 mmol) in acetonitrile (20 mL) was added 2-bromo-1-(2-chlorophenyl)ethan-1-one (1.00 g, 4.28 mmol) portionwise at room temperature. The reaction mixture was subjected to microwave heating for 20 minutes at 90° C. The mixture was cooled to room temperature and concentrated under reduced pressure. The residue was purified by reverse phase flash chromatography with the following conditions: column, C18 silica gel; mobile phase: MeOH in water (plus 10 mmol/L NH₄HCO₃), 40% to 60% gradient in 25 min; Detector: UV 220/254 nm. The desired fractions were collected and concentrated under reduced pressure to afford N-(4-(2-chlorophenyl)-1H-imidazol-2-yl)acetamide as an off-white solid.

Yield 0.50 g (50%). ¹H NMR (400 MHz, DMSO) δ 11.75 (br s, 1H), 11.31 (br s, 1H), 8.03 (d, J=7.6 Hz, 1H), 7.46 (d, J=8.0 Hz, 1H), 7.43 (s, 1H), 7.39-7.33 (m, 1H), 7.24-7.18 (m, 1H), 2.09 (s, 3H). m/z: [ESI⁺]236, 238 (M+H)⁺.

### Synthesis of 4-(2-chlorophenyl)-1H-imidazol-2-amine

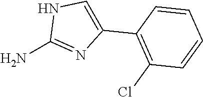 [SMILES: Nc1nc(-c2ccccc2Cl)c[nH]1]

To a stirred solution of N-(4-(2-chlorophenyl)-1H-imidazol-2-yl)acetamide (1.00 g, 4.24 mmol) in methanol (5 mL) and water (5 mL) was added concentrated sulfuric acid (2.5 mL) dropwise at 0° C. The resulting solution was subjected to microwave heating for 30 min at 100° C. The resulting mixture was cooled to room temperature and was basified to pH=8 with saturated aqueous sodium carbonate. The resulting mixture was filtered and the filter cake was washed with water (3×3 mL). Following air drying, 4-(2-chlorophenyl)-1H-imidazol-2-amine was isolated as a brown solid.

Yield 0.50 g (61%). ¹H NMR (400 MHz, CD₃OD) δ 7.70 (d, J=8.0 Hz, 1H), 7.46-7.33 (m, 2H), 7.28 (d, J=7.6 Hz, 1H), 7.17 (s, 1H). Imidazole NH and amido NH protons not observed. m/z: [ESI⁺] 194, 196 (M+H)⁺.

### Synthesis of 4-(2-(methoxymethyl)phenyl)thiazol-2-amine

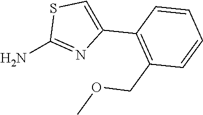

Compound 4-(2-(methoxymethyl)phenyl)thiazol-2-amine was prepared from 2-bromo-1-(2-(methoxymethyl)phenyl)ethan-1-one (243 mg, 1.00 mmol) and thiourea (91 mg, 1.20 mmol) following a procedure similar to that described for the synthesis of 4-(pyridin-2-yl)thiazol-2-amine, and was isolated as an off-white solid.

Yield 213 mg (97%). m/z: [ESI⁺] 221 (M+H)⁺.

### Synthesis of N-(4-(2-bromophenyl)thiazol-2-yl)-5-(4-(methylsulfonyl)piperazin-1-yl)picolinamide

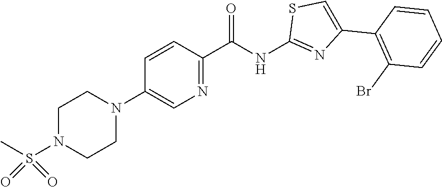 [SMILES: CS(=O)(=O)N1CCN(c2ccc(C(=O)Nc3nc(-c4ccccc4Br)cs3)nc2)CC1]

Compound N-(4-(2-bromophenyl)thiazol-2-yl)-5-(4-(methylsulfonyl)piperazin-1-yl)picolinamide was prepared from 5-(4-(methylsulfonyl)piperazin-1-yl)picolinic acid (3.00 g, 10.51 mmol) and 4-(2-bromophenyl)thiazol-2-amine (2.68 g, 10.50 mmol) following a procedure similar to that described for the synthesis of (1r,3r)-3-((4-(2-chlorophenyl)thiazol-2-yl)carbamoyl)cyclobutane-1-carboxylate and was isolated as a yellow solid.

Yield 2.60 g (47%). ¹H NMR (400 MHz, CDCl₃) δ 11.07 (br s, 1H), 8.28 (d, J=2.8 Hz, 1H), 8.17 (d, J=8.8 Hz, 1H), 7.77 (dd, J=1.6, 7.6 Hz, 1H), 7.69 (dd, J=1.2, 8.0 Hz, 1H), 7.44 (s, 1H), 7.42-7.37 (m, 1H), 7.30 (dd, J=2.8, 8.8 Hz, 1H), 7.25-7.19 (m, 1H), 3.56-3.50 (m, 4H), 3.46-3.40 (m, 4H), 2.87 (s, 3H). m/z: [ESI⁺]522, 524 (M+H)⁺.

### Synthesis of 5-(4-(methylsulfonyl)piperazin-1-yl)-N-(4-(2-vinylphenyl)thiazol-2-yl)picolinamide

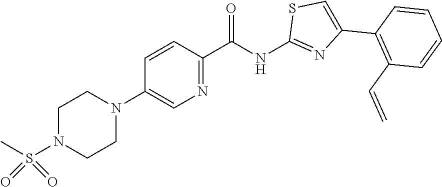 [SMILES: C=Cc1ccccc1-c1csc(NC(=O)c2ccc(N3CCN(S(C)(=O)=O)CC3)cn2)n1]

Compound 5-(4-(methylsulfonyl)piperazin-1-yl)-N-(4-(2-vinylphenyl)thiazol-2-yl)picolinamide was prepared from N-(4-(2-bromophenyl)thiazol-2-yl)-5-(4-(methylsulfonyl)piperazin-1-yl)picolinamide (2.50 g, 4.79 mmol) and potassium vinyltrifluoroborate (1.28 g, 9.56 mmol) following a procedure similar to that described for the synthesis of tert-butyl (4-(2,4-dichlorophenyl)thiazol-2-yl)carbamate and was isolated as a yellow solid.

Yield 0.49 g (22%). ¹H NMR (400 MHz, CDCl₃) δ 511.06 (br s, 1H), 8.29 (d, J=2.8 Hz, 1H), 8.19 (d, J=8.8 Hz, 1H), 7.69-7.60 (m, 2H), 7.39-7.35 (m, 2H), 7.31 (dd, J=2.8, 8.8 Hz, 1H), 7.09 (dd, J=10.8, 17.6 Hz, 1H), 7.01 (s, 1H), 5.75 (dd, J=1.6, 17.6 Hz, 1H), 5.32 (dd, J=1.6, 10.8 Hz, 1H), 3.56-3.49 (m, 4H), 3.48-3.37 (m, 4H), 2.87 (s, 3H). m/z: [ESI⁺] 470 (M+H)⁺.

### Synthesis of N-(4-(2-formylphenyl)thiazol-2-yl)-S-(4-(methylsulfonyl)piperazin-1-yl)picolinamide

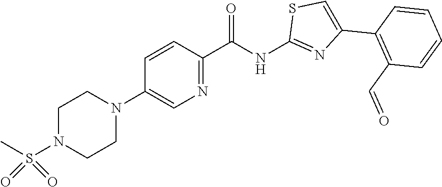 [SMILES: CS(=O)(=O)N1CCN(c2ccc(C(=O)Nc3nc(-c4ccccc4C=O)cs3)nc2)CC1]

Compound N-(4-(2-formylphenyl)thiazol-2-yl)-5-(4-(methylsulfonyl)piperazin-1-yl)picolinamide was prepared from 5-(4-(methylsulfonyl)piperazin-1-yl)-N-(4-(2-vinylphenyl)thiazol-2-yl)picolinamide (100 mg, 0.213 mmol) following a procedure similar to that described for the synthesis of benzyl bis(2-oxoethyl)carbamate and was isolated as a yellow solid.

Yield 9 mg (9%). ¹H NMR (400 MHz, CDCl₃) δ 11.02 (br s, 1H), 10.41 (s, 1H), 8.31 (d, J=2.8 Hz, 11H), 8.19 (d, J=8.8 Hz, 1H), 8.02 (dd, J=1.6, 7.8 Hz, 1H), 7.74-7.69 (m, 1H), 7.69-7.63 (m, 1H), 7.58-7.45 (m, 1H), 7.32 (dd, J=2.8, 8.8 Hz, 1H), 7.16 (s, 1H), 3.60-3.51 (m, 4H), 3.49-3.40 (m, 4H), 2.88 (s, 3H). m/z: [ESI⁺] 472 (M+H)⁺.

### Synthesis of N-(4-(2-(hydroxymethyl)phenyl)thiazol-2-yl)-4-morpholinobenzamide

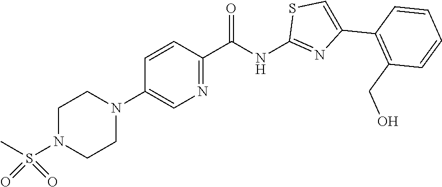 [SMILES: CS(=O)(=O)N1CCN(c2ccc(C(=O)Nc3nc(-c4ccccc4CO)cs3)nc2)CC1]

Compound N-(4-(2-(hydroxymethyl)phenyl)thiazol-2-yl)-4-morpholinobenzamide was prepared from N-(4-bromothiazol-2-yl)-4-morpholinobenzamide (2.40 g, 6.52 mmol) and (2-(hydroxymethyl)phenyl)boronic acid (1.19 g, 7.83 mmol) following a procedure similar to that described for the synthesis of methyl 4-(3,6-dihydro-2H-thiopyran-4-yl)benzoate and was isolated as an orange solid. Yield 2.50 g (97%). ¹H NMR (400 MHz, CDCl₃) δ 11.01 (br s, 1H), 8.03 (d, J=8.8 Hz, 2H), 7.59 (dd, J=1.6, 7.6 Hz, 1H), 7.36-7.30 (m, 1H), 7.25-7.20 (m, 1H), 7.08 (s, 1H), 6.97-6.88 (m, 3H), 4.52 (s, 2H), 3.94-3.87 (m, 4H), 3.39-3.29 (m, 4H). aliphatic OH proton not observed. m/z: [ESI⁺] 396 (M+H)⁺.

### Synthesis of (3-bromopyridin-2-yl)methyl acetate

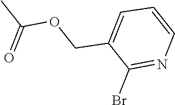

To a stirred solution of (3-bromopyridin-2-yl)methanol (2.00 g, 10.64 mmol) in THF (40 mL) were added acetic anhydride (1.64 g, 16.06 mmol), pyridine (1.60 g, 20.23 mmol) and DMAP (0.13 g, 1.06 mmol), at room temperature under a nitrogen atmosphere. The resulting solution was stirred overnight at room temperature. The resulting mixture was concentrated under reduced pressure. The residue was purified by silica gel column chromatography, eluting with 30% ethyl acetate in petroleum ether to afford (3-bromopyridin-2-yl)methyl acetate as a colorless oil.

Yield 1.93 g (79%). ¹H NMR (400 MHz, CDCl₃) δ 8.55 (dd, J=1.6, 4.8 Hz, 1H), 7.88 (dd, J=1.6, 8.0 Hz, 1H), 7.15 (dd, J=4.8, 8.0 Hz, 1H), 5.33 (s, 2H), 2.18 (s, 3H). m/z: [ESI⁺] 230, 232 (M+H)⁺.

### Synthesis of (3-(4,4,5,5-tetramethyl-1,3,2-dioxaborolan-2-yl)pyridin-2-yl)methyl acetate

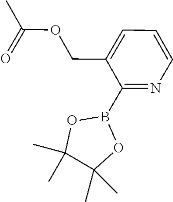 [SMILES: CC(=O)OCc1cccnc1B1OC(C)(C)C(C)(C)O1]

To a stirred solution of (3-bromopyridin-2-yl)methyl acetate (1.00 g, 4.35 mmol) in 1,4-dioxane (10 mL) were added bis(pinacolato)diboron (1.63 g, 6.42), potassium acetate (1.29 g, 13.14 mmol) and [1,1′-bis(diphenylphosphino)ferrocene]dichloropalladium(II) (0.16 g, 0.22 mmol) at room temperature under a nitrogen atmosphere. The resulting mixture was stirred overnight at 100° C. under a nitrogen atmosphere. The resulting mixture was cooled to room temperature and diluted with water (50 mL). The resulting mixture was extracted with ethyl acetate (3×30 mL). The combined organic layers were dried over anhydrous Na₂SO₄. After filtration, the filtrate was concentrated under reduced pressure. The residue was purified by silica gel column chromatography, eluting with 16% methanol in DCM to afford (3-(4,4,5,5-tetramethyl-1,3,2-dioxaborolan-2-yl)pyridin-2-yl)methyl acetate as a black oil.

Yield 375 mg (31%). ¹H NMR (400 MHz, CDCl₃) δ 8.67 (dd, J=2.0, 4.8 Hz, 11H), 8.13 (dd, J=2.0, 7.6 Hz, 1H), 7.26 (dd, J=4.8, 7.6 Hz, 1H), 5.46 (s, 2H), 2.14 (s, 3H), 1.36 (s, 12H). m/z: [ESI⁺] 278 (M+H)⁺.

### Synthesis of 2-(methoxymethyl)-3-(4,4,5,5-tetramethyl-1,3,2-dioxaborolan-2-yl)pyridine

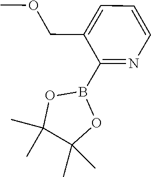 [SMILES: COCc1cccnc1B1OC(C)(C)C(C)(C)O1]

Compound 2-(methoxymethyl)-3-(4,4,5,5-tetramethyl-1,3,2-dioxaborolan-2-yl)pyridine was prepared from 3-bromo-2-(methoxymethyl)pyridine (1.00 g, 4.95 mmol) and bis(pinacolato)diboron (1.89 g, 7.42 mmol) following a procedure similar to that described for the synthesis of (3-(4,4,5,5-tetramethyl-1,3,2-dioxaborolan-2-yl)pyridin-2-yl)methyl acetate, and was isolated as a black oil.

Yield 428 mg (35%). ¹H NMR (400 MHz, CDCl₃) δ 8.63 (d, J=2.0 Hz, 1H), 8.02 (d, J=7.6 Hz, 1H), 7.20 (dd, J=2.0, 7.6 Hz, 1H), 4.78 (s, 2H), 3.46 (s, 3H), 1.38 (s, 12H). m/z: [ESI⁺] 250 (M+H)⁺.

### Synthesis of methyl 5-(4-acetylpiperazin-1-yl)picolinate

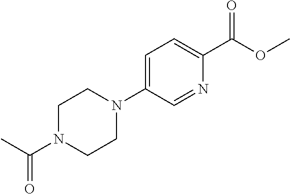 [SMILES: COC(=O)c1ccc(N2CCN(C(C)=O)CC2)cn1]

Compound methyl 5-(4-acetylpiperazin-1-yl)picolinate was prepared from methyl 5-(piperazin-1-yl)picolinate hydrochloride (15.00 g, 58.20 mmol) following a procedure similar to that described for the synthesis of (3-bromopyridin-2-yl)methyl acetate with modified condition using DMF as a solvent and triethylamine as a base. Product was isolated as an off-white solid.

Yield 10.28 g (67%). ¹H NMR (400 MHz, CDCl₃) δ 8.40 (d, J=2.8 Hz, 1H), 8.03 (d, J=8.8 Hz, 1H), 7.18 (dd, J=2.8, 8.8 Hz, 1H), 3.97 (s, 3H), 3.84-3.79 (m, 2H), 3.71-3.65 (m, 2H), 3.46-3.41 (m, 2H), 3.40-3.35 (m, 2H), 2.17 (s, 3H). m/z: [ESI⁺] 264 (M+H)⁺.

### Synthesis of 5-(4-acetylpiperazin-1-yl)picolinic acid

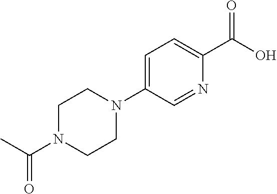 [SMILES: CC(=O)N1CCN(c2ccc(C(=O)O)nc2)CC1]

Compound 5-(4-acetylpiperazin-1-yl)picolinic acid was prepared from methyl 5-(4-acetylpiperazin-1-yl)picolinate (10.00 g, 37.98 mmol) following a procedure similar to that described for the synthesis of 4-(1,1-dioxidotetrahydro-2H-thiopyran-4-yl)benzoic acid, and was isolated as an off-white solid.

Yield 4.20 g (44%). ¹H NMR (400 MHz, DMSO) δ 8.35 (d, J=2.8 Hz, 1H), 7.87 (d, J=8.8 Hz, 1H), 7.35 (dd, J=2.8, 8.8 Hz, 1H), 3.65-3.51 (m, 4H), 3.40 (d, J=5.6 Hz, 2H), 3.33 (d, J=5.6 Hz, 2H), 2.05 (s, 3H). Carboxylic acid OH proton not observed. m/z: [ESI⁺] 250 (M+H)⁺.

### Synthesis of 5-(4-acetylpiperazin-1-yl)-N-(4-bromothiazol-2-yl)picolinamide

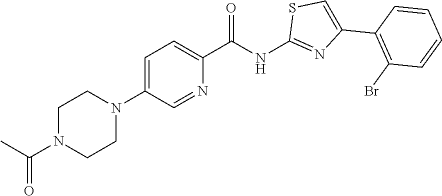 [SMILES: CC(=O)N1CCN(c2ccc(C(=O)Nc3nc(-c4ccccc4Br)cs3)nc2)CC1]

Compound 5-(4-acetylpiperazin-1-yl)-N-(4-bromothiazol-2-yl)picolinamide was prepared from 5-(4-acetylpiperazin-1-yl)picolinic acid (4.40 g, 17.65 mmol) and 4-bromothiazol-2-amine (4.08 g, 22.79 mmol) following a procedure similar to that described for the synthesis of N-(4-(2-chlorophenyl)thiazol-2-yl)-5-fluoropicolinamide and was isolated as a yellow solid.

Yield 4.37 g (60%). ¹H NMR (400 MHz, CDCl₃) δ 511.01 (br s, 1H), 8.25 (d, J=2.8 Hz, 1H), 8.13 (d, J=8.8 Hz, 1H), 7.26 (dd, J=2.8, 8.8 Hz, 1H), 6.91 (s, 1H), 3.88-3.79 (m, 2H), 3.73-3.65 (m, 2H), 3.50-3.45 (m, 2H), 3.45-3.37 (m, 2H), 2.18 (s, 3H). m/z: [ESI⁺] 410, 412 (M+H)⁺.

### Synthesis of 5-bromo-N-(4-(2-chlorophenyl)thiazol-2-yl)picolinamide

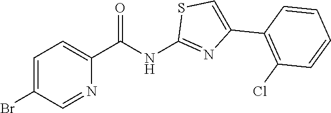 [SMILES: O=C(Nc1nc(-c2ccccc2Cl)cs1)c1ccc(Br)cn1]

Compound 5-bromo-N-(4-(2-chlorophenyl)thiazol-2-yl)picolinamide was prepared from 5-bromopicolinic acid (5.00 g, 24.75 mmol) and 4-(2-chlorophenyl)thiazol-2-amine (7.82 g, 37.12 mmol) following a procedure similar to that described for the synthesis of N-(4-(2-chlorophenyl)thiazol-2-yl)-5-fluoropicolinamide and was isolated as a light yellow solid.

Yield 9.41 g (96%). ¹H NMR (400 MHz, DMSO) δ 12.29 (br s, 1H), 8.91 (s, 1H), 8.38 (d, J=8.4 Hz, 1H), 8.12 (d, J=8.4 Hz, 1H), 7.92 (d, J=7.6 Hz, 1H), 7.77 (s, 1H), 7.57 (d, J=7.6 Hz, 1H), 7.50-7.34 (m, 2H). m/z: [ESI⁺] 394, 396, 398 (M+H)⁺.

### Synthesis of tert-butyl 4-(6-((4-(2-chlorophenyl)thiazol-2-yl)carbamoyl)nicotinoyl)piperazine-1-carboxylate

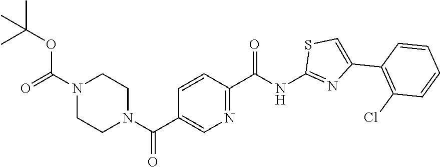 [SMILES: CC(C)(C)OC(=O)N1CCN(C(=O)c2ccc(C(=O)Nc3nc(-c4ccccc4Cl)cs3)nc2)CC1]

To a mixture of 5-bromo-N-(4-(2-chlorophenyl)thiazol-2-yl)picolinamide (50 mg, 0.13 mmol) and tert-butyl piperazine-1-carboxylate (59 mg, 0.32 mmol) in DMF (2 mL) were added triethylamine (51 mg, 0.51 mmol), palladium (II) acetate (6 mg, 0.03 mmol) and 9,9-dimethyl-4,5-bis(diphenylphosphino)xanthene (15 mg, 0.03 mmol) at room temperature under a nitrogen atmosphere. The resulting mixture was stirred for 16 h at 100° C. under a carbon monoxide atmosphere (1.5 atmosphere pressure). The resulting mixture was cooled to room temperature and purified by reverse phase flash chromatography with the following conditions: Column: WelFlash TM C18-L, 20-40 μm, 120 g; Eluent A: water (plus 10 mmol/L NH₄HCO₃); Eluent B: acetonitrile; Gradient: 60%-80% B in 20 min; Flow rate: 60 mL/min; Detector: UV 220/254 nm. The desired fractions were collected and concentrated under reduced pressure to afford tert-butyl 4-(6-((4-(2-chlorophenyl)thiazol-2-yl)carbamoyl)nicotinoyl)piperazine-1-carboxylate as an off-white solid.

Yield 30 mg (45%). ¹H NMR (400 MHz, DMSO) δ 12.29 (br s, 1H), 8.81 (dd, J=0.8, 2.0 Hz, 1H), 8.26 (dd, J=0.8, 8.0 Hz, 1H), 8.15 (dd, J=2.0, 8.0 Hz, 1H), 7.93 (dd, J=2.0, 7.6 Hz, 1H), 7.78 (s, 1H), 7.58 (dd, J=1.6, 7.6 Hz, 1H), 7.49-7.38 (m, 2H), 3.52-3.42 (m, 4H), 3.42-3.31 (m, 4H), 1.42 (s, 9H). m/z: [ESI⁺] 528, 530 (M+H)⁺.

### Synthesis of tert-butyl (1-(6-((4-(2-chlorophenyl)thiazol-2-yl)carbamoyl)pyridin-3-yl)piperidin-4-yl)carbamate

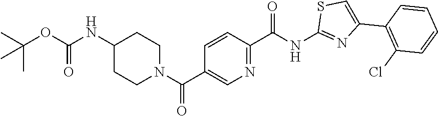 [SMILES: CC(C)(C)OC(=O)NC1CCN(C(=O)c2ccc(C(=O)Nc3nc(-c4ccccc4Cl)cs3)nc2)CC1]

Compound tert-butyl (1-(6-((4-(2-chlorophenyl)thiazol-2-yl)carbamoyl)pyridin-3-yl)piperidin-4-yl)carbamate was prepared from 5-bromo-N-(4-(2-chlorophenyl)thiazol-2-yl)picolinamide (0.40 g, 1.01 mmol) and tert-butyl piperidin-4-ylcarbamate (0.30 g, 1.50 mmol) following a procedure similar to that described for the synthesis of tert-butyl 4-(6-((4-(2-chlorophenyl)thiazol-2-yl)carbamoyl)nicotinoyl)piperazine-1-carboxylate and was isolated as an off-white solid.

Yield 0.12 g (23%). ¹H NMR (400 MHz, CDCl₃) δ 11.07 (br s, 1H), 8.26 (d, J=2.8 Hz, 1H), 8.14 (d, J=8.8 Hz, 1H), 7.94 (dd, J=1.6, 7.6 Hz, 1H), 7.53 (s, 1H), 7.49 (dd, J=1.6, 7.6 Hz, 1H), 7.39-7.34 (m, 1H), 7.31-7.24 (m, 2H), 4.51 (br s, 1H), 3.94-3.83 (m, 2H), 3.79-3.67 (m, 1H), 3.16-2.98 (m, 2H), 2.19-2.08 (m, 2H), 1.60-1.49 (m, 2H), 1.48 (s, 9H). m/z: [ESI⁺] 514, 516 (M+H)⁺.

### Synthesis of N-(4-bromothiazol-2-yl)-5-fluoropicolinamide

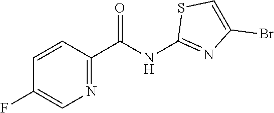 [SMILES: O=C(Nc1nc(Br)cs1)c1ccc(F)cn1]

Compound N-(4-bromothiazol-2-yl)-5-fluoropicolinamide was prepared from 5-fluoropicolinic acid (35.94 g, 254.71 mmol) and 4-bromothiazol-2-amine (38.00 g, 212.24 mmol) following a procedure similar to that described for the synthesis of N-(4-(2-chlorophenyl)thiazol-2-yl)-5-fluoropicolinamide and was isolated as a light yellow solid.

Yield 55.00 g (86%). ¹H NMR (400 MHz, CDCl₃) δ 11.04 (br s, 1H), 8.56-8.46 (m, 1H), 8.39-8.28 (m, 111), 7.74-7.60 (m, 1H), 6.96 (s, 1H). m/z: [ESI⁺] 302, 304 (M+H)⁺.

### Synthesis of tert-butyl 4-(6-((4-bromothiazol-2-yl)carbamoyl)pyridin-3-yl)piperazine-1-carboxylate

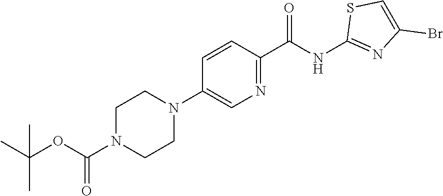 [SMILES: CC(C)(C)OC(=O)N1CCN(c2ccc(C(=O)Nc3nc(Br)cs3)nc2)CC1]

Compound tert-butyl 4-(6-((4-bromothiazol-2-yl)carbamoyl)pyridin-3-yl)piperazine-1-carboxylate was prepared from N-(4-bromothiazol-2-yl)-5-fluoropicolinamide (25.00 g, 82.75 mmol) and tert-butyl piperazine-1-carboxylate (18.49 g, 99.27 mmol) following a procedure similar to that described for the synthesis of 2-chloro-4-morpholinobenzaldehyde and was isolated as a yellow solid.

Yield 30.00 g (77%). ¹H NMR (400 MHz, DMSO) δ 511.99 (br s, 1H), 8.40 (d, J=2.8 Hz, 1H), 7.99 (d, J=8.8 Hz, 1H), 7.47 (dd, J=2.8, 8.8 Hz, 1H), 7.36 (s, 1H), 3.53-3.47 (m, 4H), 3.47-3.40 (m, 4H), 1.43 (s, 9H). m/z: [ESI⁺] 468, 470 (M+H)⁺.

### Synthesis of N-(4-bromothiazol-2-yl)-5-(piperazin-1-yl)picolinamide hydrochloride

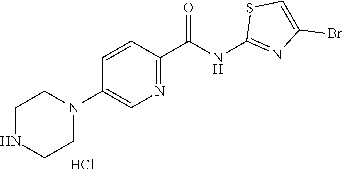 [SMILES: O=C(Nc1nc(Br)cs1)c1ccc(N2CCNCC2)cn1 ; Cl]

Compound N-(4-bromothiazol-2-yl)-5-(piperazin-1-yl)picolinamide hydrochloride was prepared from tert-butyl 4-(6-((4-bromothiazol-2-yl)carbamoyl)pyridin-3-yl)piperazine-1-carboxylate (468 mg, 1.00 mmol) following a procedure similar to that described for the synthesis of methyl 5-(piperazin-1-yl)picolinate hydrochloride and was isolated as an off-white solid.

Yield 354 mg (88%). m/z: [ESI⁺] 368, 370 (M+H)⁺.

### Synthesis of 5-(4-acetylpiperazin-1-yl)-N-(4-bromothiazol-2-yl)picolinamide (Method 2)

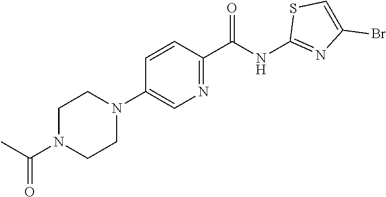 [SMILES: CC(=O)N1CCN(c2ccc(C(=O)Nc3nc(Br)cs3)nc2)CC1]

Compound 5-(4-acetylpiperazin-1-yl)-N-(4-bromothiazol-2-yl)picolinamide was prepared from N-(4-bromothiazol-2-yl)-5-(piperazin-1-yl)picolinamide hydrochloride (300 mg, 0.74 mmol) following a procedure similar to that described for the synthesis of (3-bromopyridin-2-yl)methyl acetate with modified condition using DMF as a solvent and triethylamine as a base. Product was isolated as an off-white solid.

Yield 268 mg (88%). ¹H NMR (400 MHz, CDCl₃) δ 11.01 (s, 1H), 8.25 (d, J=2.8 Hz, 1H), 8.13 (d, J=8.8 Hz, 1H), 7.26 (dd, J=2.8, 8.8 Hz, 1H), 6.91 (s, 1H), 3.80-3.88 (m, 2H), 3.67-3.74 (m, 2H), 3.52-3.37 (m, 4H), 2.18 (s, 3H). m/z: [ESI⁺] 410, 412 (M+H)⁺.

### Synthesis of 5-bromo-N-(4-(2-chlorophenyl)thiazol-2-yl)-3-methylpicolinamide

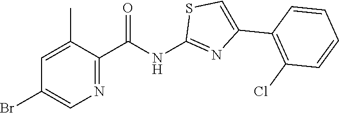 [SMILES: Cc1cc(Br)cnc1C(=O)Nc1nc(-c2ccccc2Cl)cs1]

Compound 5-bromo-N-(4-(2-chlorophenyl)thiazol-2-yl)-3-methylpicolinamide was prepared from 5-bromo-3-methylpicolinic acid (315 mg, 1.46 mmol) and 4-(2-chlorophenyl)thiazol-2-amine (399 mg, 1.89 mmol) following a procedure similar to that described for the synthesis of N-(4-(2-chlorophenyl)thiazol-2-yl)-5-fluoropicolinamide and was isolated as an off-white solid.

Yield 447 mg (75%). ¹H NMR (400 MHz, DMSO) δ 12.36 (br s, 1H), 8.70 (d, J=2.0 Hz, 1H), 8.22 (d, J=2.0 Hz, 1H), 7.89 (dd, J=2.0, 7.6 Hz, 1H), 7.74 (s, 1H), 7.57 (dd, J=1.6, 7.6 Hz, 1H), 7.50-7.35 (m, 2H), 2.59 (s, 3H). m/z: [ESI⁺] 408, 410, 412 (M+H)⁺.

### Synthesis of 4-bromo-N-(4-(2-chlorophenyl)thiazol-2-yl)thiophene-2-carboxamide

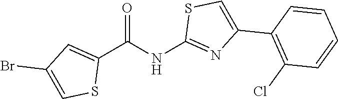 [SMILES: O=C(Nc1nc(-c2ccccc2Cl)cs1)c1cc(Br)cs1]

Compound 4-bromo-N-(4-(2-chlorophenyl)thiazol-2-yl)thiophene-2-carboxamide was prepared from 4-bromothiophene-2-carboxylic acid (500 mg, 2.42 mmol) and 4-(2-chlorophenyl)thiazol-2-amine (660 mg, 3.13 mmol) following a procedure similar to that described for the synthesis of N-(4-(2-chlorophenyl)thiazol-2-yl)-5-fluoropicolinamide and was isolated as a yellow solid.

Yield 600 mg (62%). ¹H NMR (400 MHz, CDCl₃) δ 11.13 (br s, 1H), 7.71-7.63 (m, 1H), 7.46 (d, J=2.8 Hz, 1H), 7.45-7.33 (m, 3H), 7.26-7.18 (m, 2H). m/z: [ESI⁺] 399, 401, 403 (M+H)⁺.

### Synthesis of 5-bromo-N-(4-(2-chlorophenyl)thiazol-2-yl)thiophene-2-carboxamide

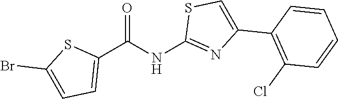 [SMILES: O=C(Nc1nc(-c2ccccc2Cl)cs1)c1ccc(Br)s1]

Compound 5-bromo-N-(4-(2-chlorophenyl)thiazol-2-yl)thiophene-2-carboxamide was prepared from 5-bromothiophene-2-carboxylic acid (500 mg, 2.42 mmol) and 4-(2-chlorophenyl)thiazol-2-amine (660 mg, 3.13 mmol) following a procedure similar to that described for the synthesis of N-(4-(2-chlorophenyl)thiazol-2-yl)-5-fluoropicolinamide and was isolated as a yellow solid.

Yield 873 mg (90%). ¹H NMR (400 MHz, CDCl₃) δ 11.98 (br s, 1H), 7.57 (dd, J=2.0, 7.6 Hz, 1H), 7.43 (s, 1H), 7.34 (dd, J=1.6, 7.6 Hz, 1H), 7.23-7.12 (m, 2H), 7.05 (d, J=4.0 Hz, 1H), 6.84 (d, J=4.0 Hz, 1H). m/z: [ESI⁺] 399, 401, 403 (M+H)⁺.

### Synthesis of 4-bromo-N-(4-(2-chlorophenyl)thiazol-2-yl)-2-methylbenzamide

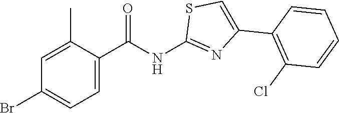 [SMILES: Cc1cc(Br)ccc1C(=O)Nc1nc(-c2ccccc2Cl)cs1]

Compound 4-bromo-N-(4-(2-chlorophenyl)thiazol-2-yl)-2-methylbenzamide was prepared from 4-bromo-2-methylbenzoic acid (500 mg, 2.33 mmol) and 4-(2-chlorophenyl)thiazol-2-amine (640 mg, 3.04 mmol) following a procedure similar to that described for the synthesis of N-(4-(2-chlorophenyl)thiazol-2-yl)-5-fluoropicolinamide and was isolated as a yellow solid.

Yield 900 mg (95%). ¹H NMR (400 MHz, CDCl₃) δ 12.19 (br s, 1H), 7.53 (dd, J=2.0, 7.6 Hz, 1H), 7.45 (s, 1H), 7.37 (dd, J=1.6, 7.6 Hz, 1H), 7.24-7.13 (m, 4H), 7.10 (d, J=8.0 Hz, 1H), 2.35 (s, 3H). m/z: [ESI⁺] 407, 409, 411 (M+H)⁺.

### Synthesis of N-(4-bromothiazol-2-yl)-5-(4-(methylsulfonyl)piperazin-1-yl)picolinamide

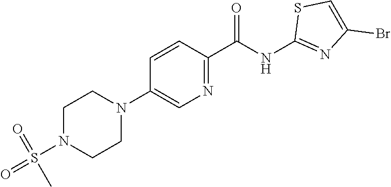 [SMILES: CS(=O)(=O)N1CCN(c2ccc(C(=O)Nc3nc(Br)cs3)nc2)CC1]

Compound N-(4-bromothiazol-2-yl)-5-(4-(methylsulfonyl)piperazin-1-yl)picolinamide was prepared from 5-(4-(methylsulfonyl)piperazin-1-yl)picolinic acid (900 mg, 3.15 mmol) and 4-bromothiazol-2-amine (847 mg, 4.73 mmol) following a procedure similar to that described for the synthesis of N-(4-(2-chlorophenyl)thiazol-2-yl)-5-fluoropicolinamide and was isolated as an off-white solid.

Yield 800 mg (57%). ¹H NMR (400 MHz, CDCl₃) δ 11.02 (br s, 1H), 8.29 (d, J=2.8 Hz, 1H), 8.16 (d, J=8.8 Hz, 1H), 7.31 (dd, J=2.8, 8.8 Hz, 1H), 6.92 (s, 1H), 3.57-3.52 (m, 4H), 3.48-3.41 (m, 4H), 2.88 (s, 3H). m/z: [ESI⁺] 446, 448 (M+H)⁺.

### Synthesis of N-(4-(2-chlorophenyl)thiazol-2-yl)-3-oxocyclobutane-1-carboxamide

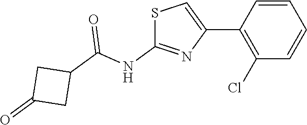 [SMILES: O=C1CC(C(=O)Nc2nc(-c3ccccc3Cl)cs2)C1]

Compound N-(4-(2-chlorophenyl)thiazol-2-yl)-3-oxocyclobutane-1-carboxamide was prepared from 3-oxocyclobutane-1-carboxylic acid (380 mg, 3.33 mmol) and 4-(2-chlorophenyl)thiazol-2-amine (500 mg, 2.37 mmol) following a procedure similar to that described for the synthesis of methyl (1r,3r)-3-((4-(2-chlorophenyl)thiazol-2-yl)carbamoyl)cyclobutane-1-carboxylate and was isolated as a yellow semi-solid.

Yield 400 mg (55%). ¹H NMR (400 MHz, DMSO) δ 12.58 (br s, 1H), 7.83 (dd, J=2.0, 7.6 Hz, 1H), 7.63 (s, 1H), 7.56 (dd, J=1.6, 7.6 Hz, 1H), 7.47-7.35 (m, 2H), 3.55-3.45 (m, 1H), 3.35-3.34 (m, 2H), 3.33-3.32 (m, 2H). m/z: [ESI⁺] 307, 309 (M+H)⁺.

### Synthesis of N-(4-(2-chlorophenyl)thiazol-2-yl)-4-oxocyclohexane-1-carboxamide

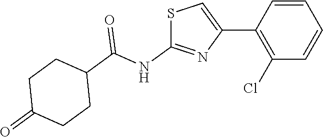 [SMILES: O=C1CCC(C(=O)Nc2nc(-c3ccccc3Cl)cs2)CC1]

Compound N-(4-(2-chlorophenyl)thiazol-2-yl)-4-oxocyclohexane-1-carboxamide was prepared from 4-oxocyclohexane-1-carboxylic acid (0.47 g, 3.31 mmol) and 4-(2-chlorophenyl)thiazol-2-amine (0.50 g, 2.37 mmol) following a procedure similar to that described for the synthesis of methyl (1r,3r)-3-((4-(2-chlorophenyl)thiazol-2-yl)carbamoyl)cyclobutane-1-carboxylate and was isolated as an off-white solid.

Yield 0.60 g (76%). ¹H NMR (400 MHz, DMSO) δ 12.42 (br s, 1H), 7.84 (dd, J=1.6, 7.6 Hz, 1H), 7.61 (s, 1H), 7.56 (d, J=7.6 Hz, 1H), 7.48-7.33 (m, 2H), 3.05-2.92 (m, 1H), 2.49-2.38 (m, 2H), 2.37-2.28 (m, 2H), 2.22-2.13 (m, 2H), 1.96-1.83 (m, 2H). m/z: [ESI⁺] 335, 337 (M+H)⁺.

### Synthesis of ethyl 3-((tert-butyldiphenylsilyl)oxy)propanoate

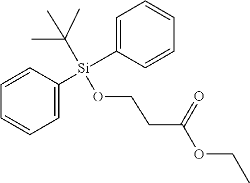 [SMILES: CCOC(=O)CCO[Si](c1ccccc1)(c1ccccc1)C(C)(C)C]

To a solution of ethyl 3-hydroxypropanoate (1.50 g 12.70 mmol) and imidazole (2.59 g, 38.04 mmol) in DCM (20 mL) was added tert-butylchlorodiphenylsilane (4.19 g 15.24 mmol) at room temperature under a nitrogen atmosphere. The resulting solution was stirred for 2 days at room temperature under a nitrogen atmosphere. The resulting mixture was concentrated under reduced pressure. The residue was purified by silica gel column chromatography, eluted with 0-10% ethyl acetate in petroleum ether, to afford ethyl 3-((tert-butyldiphenylsilyl)oxy)propanoate as a colorless oil.

Yield 3.80 g (84%). No ¹H NMR and MS data available.

### Synthesis of 3-((tert-butyldiphenylsilyl)oxy)propanoic acid

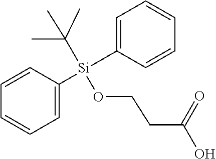 [SMILES: CC(C)(C)[Si](OCCC(=O)O)(c1ccccc1)c1ccccc1]

Compound 3-((tert-butyldiphenylsilyl)oxy)propanoic acid was prepared from ethyl 3-((tert-butyldiphenylsilyl)oxy)propanoate (3.80 g, 10.66 mmol) and lithium hydroxide monohydrate (2.24 g, 53.38 mmol), following a procedure similar to that described for the synthesis of 4-(1,1-dioxidotetrahydro-2H-thiopyran-4-yl)benzoic acid and was isolated as a colorless oil.

Yield 2.50 g (71%). ¹H NMR (400 MHz, CDCl₃) δ 7.71 (dd, J=1.6, 8.0 Hz, 4H), 7.50-7.37 (m, 6H), 3.99 (t, J=6.4 Hz, 2H), 2.64 (t, J=6.4 Hz, 2H), 1.08 (s, 9H). Carboxylic acid OH proton not observed. No MS data available.

### Synthesis of 5-(4-(3-((tert-butyldiphenylsilyl)oxy)propanoyl)piperazin-1-yl)-N-(4-(2-chlorophenyl)thiazol-2-yl)picolinamide

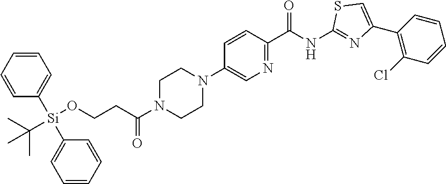 [SMILES: CC(C)(C)[Si](OCCC(=O)N1CCN(c2ccc(C(=O)Nc3nc(-c4ccccc4Cl)cs3)nc2)CC1)(c1ccccc1)c1ccccc1]

Compound 5-(4-(3-((tert-butyldiphenylsilyl)oxy)propanoyl)piperazin-1-yl)-N-(4-(2-chlorophenyl)thiazol-2-yl)picolinamide was prepared from N-(4-(2-chlorophenyl)thiazol-2-yl)-5-(piperazin-1-yl)picolinamide hydrochloride (420 mg, 0.96 mmol) and 3-((tert-butyldiphenylsilyl)oxy)propanoic acid (470 mg, 1.43 mmol), following a procedure similar to that described for the synthesis of methyl (1r,3r)-3-((4-(2-chlorophenyl)thiazol-2-yl)carbamoyl)cyclobutane-1-carboxylate and was isolated as an off-white solid.

Yield 500 mg (73%). ¹H NMR (400 MHz, CDCl₃) δ 11.04 (br s, 1H), 8.21 (d, J=2.8 Hz, 1H), 8.17 (d, J=8.8 Hz, 1H), 7.94 (dd, J=2.0, 8.0 Hz, 1H), 7.69 (dd, J=1.6, 8.0 Hz, 4H), 7.54 (s, 1H), 7.49 (dd, J=1.6, 8.0 Hz, 1H), 7.48-7.34 (m, 7H), 7.31-7.25 (m, 1H), 7.21 (dd, J=2.8, 8.8 Hz, 111), 4.08 (t, J=6.8 Hz, 2H), 3.87-3.77 (m, 2H), 3.70-3.59 (m, 2H), 3.45-3.34 (m, 2H), 3.34-3.25 (m, 2H), 2.68 (t, J=6.8 Hz, 2H), 1.08 (s, 9H). m/z: [ESI⁺] 710, 712 (M+H)⁺.

### Synthesis of N-(4-(2-chlorophenyl)thiazol-2-yl)-4-(piperazin-1-yl)benzamide (Intermediate and Compound 334)

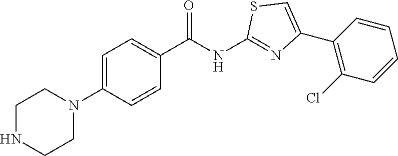 [SMILES: O=C(Nc1nc(-c2ccccc2Cl)cs1)c1ccc(N2CCNCC2)cc1]

Tert-butyl 4-(4-[[4-(2-chlorophenyl)-1,3-thiazol-2-yl]carbamoyl]phenyl)piperazine-1-carboxylate (1.70 g, 3.41 mmol) was treated with a 4 M solution of HCl in 1,4-dioxane (30 mL) for 1 h at room temperature under a nitrogen atmosphere. The resulting mixture was concentrated under reduced pressure. The residue was purified by reverse phase flash chromatography with the following conditions: Column: Spherical C18, 20-40 um, 330 g; Mobile Phase A: water (plus 10 mM NH₄HCO₃); Mobile Phase B: ACN; Flow rate: 80 mL/min; Gradient: 50% B-70% B in 20 min; Detector: UV 254/220 nm. The fractions containing desired product were collected and concentrated under reduced pressure to afford N-(4-(2-chlorophenyl)thiazol-2-yl)-4-(piperazin-1-yl)benzamide as an off-white solid:
Yield 0.76 g (56%). ¹H NMR (400 MHz, DMSO) δ 12.47 (br s, 1H), 8.10 (d, J=8.9 Hz, 2H), 7.92 (dd, J=1.9, 7.7 Hz, 1H), 7.62 (s, 1H), 7.57 (dd, J=1.4, 7.8 Hz, 1H), 7.45 (td, J=1.5, 7.5 Hz, 1H), 7.39 (td, J=1.9, 7.6 Hz, 1H), 7.01 (d, J=8.9 Hz, 2H), 3.26 (t, J=6.3 Hz, 4H), 2.83 (t, J=6.3 Hz, 4H). Piperazine NH proton not visible. m/z: [ESI⁺] 399, 401 (M+H)⁺, (C₂₀H₁₉ClN₄OS).

### Synthetic Details for Compounds of the Invention

### Synthesis of N-(4-(2,4-dichlorophenyl)thiazol-2-yl)-4-methoxybenzamide (Compound 300)

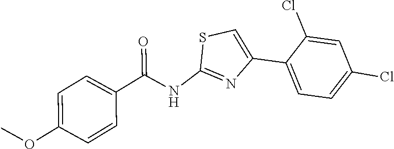 [SMILES: COc1ccc(C(=O)Nc2nc(-c3ccc(Cl)cc3Cl)cs2)cc1]

To a mixture of N-(4-bromothiazol-2-yl)-4-methoxybenzamide (120 mg, 0.383 mmol) and (2,4-dichlorophenyl)boronic acid (146 mg, 0.766 mmol) in 1,4-dioxane (3 mL) was added [1,1′-bis(diphenylphosphino)ferrocene] dichloropalladium(II), complex with dichloromethane (31 mg, 0.038 mmol), and a solution of sodium carbonate (122 mg, 1.15 mmol) in water (0.3 mL). The reaction mixture was heated at 120° C. in a microwave for 1 hour. After cooling to room temperature, the mixture was partitioned between ethyl acetate (15 ml) and brine (10 mL). The aqueous phase was extracted with ethyl acetate (2×15 mL) and the combined organic extracts were dried (MgSO₄), filtered and evaporated. The crude product was purified by preparative HPLC to afford N-(4-(2,4-dichlorophenyl)thiazol-2-yl)-4-methoxybenzamide as a colourless solid.

Yield 31 mg (21%). ¹H NMR (400 MHz, DMSO) δ 12.67 (s, 1H), 8.17-8.14 (m, 2H), 7.97 (d, J=8.4 Hz, 1H), 7.76-7.73 (m, 2H), 7.57 (dd, J=2.2, 8.5 Hz, 1H), 7.10 (d, J=8.9 Hz, 2H), 3.88 (s, 3H). m/z: [ESI⁺] 379 (M+H)⁺, (C₁₇H₁₂Cl₂N₂O₂S).

### Synthesis of N-(4-(2-chlorophenyl)thiazol-2-yl)-4-methoxybenzamide (Compound 304)

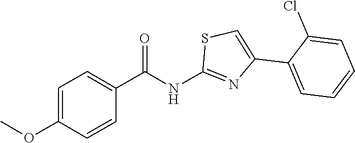 [SMILES: COc1ccc(C(=O)Nc2nc(-c3ccccc3Cl)cs2)cc1]

Compound N-(4-(2-chlorophenyl)thiazol-2-yl)-4-methoxybenzamide was prepared from (2-chlorophenyl)boronic acid following a procedure similar to that described for the synthesis of N-(4-(2,4-dichlorophenyl)thiazol-2-yl)-4-methoxybenzamide, and was isolated as an off-white solid.

Yield 34 mg (26%). ¹H NMR (400 MHz, DMSO) δ 12.65 (s, 1H), 8.16 (d, J=8.9 Hz, 2H), 7.92 (dd, J=1.8, 7.7 Hz, 1H), 7.67 (s, 1H), 7.60-7.57 (m, 1H), 7.50-7.38 (m, 2H), 7.11 (d, J=8.9 Hz, 2H), 3.88 (s, 3H). m/z: [ESI⁺] 345 (M+H)⁺, (C₁₇H₁₃ClN₂O₂S).

### Synthesis of N-(4-(2,4-dimethylphenyl)thiazol-2-yl)-4-methoxybenzamide (Compound 301)

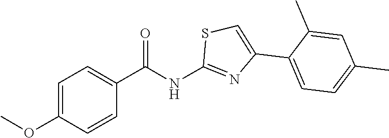

Compound N-(4-(2,4-dimethylphenyl)thiazol-2-yl)-4-methoxybenzamide was prepared from (2,4-dimethylphenyl)boronic acid following a procedure similar to that described for the synthesis of N-(4-(2,4-dichlorophenyl)thiazol-2-yl)-4-methoxybenzamide, and was isolated as a dark brown solid.

Yield 45 mg (35%). ¹H NMR (400 MHz, DMSO) δ 12.54 (br s, 1H), 8.15 (d, J=8.3 Hz, 2H), 7.55 (d, J=7.6 Hz, 1H), 7.24 (s, 1H), 7.15-7.06 (m, 4H), 3.87 (s, 3H), 2.44 (s, 3H), 2.32 (s, 3H). m/z: [ESI⁺] 339 (M+H)⁺, (C₁₉H₁₈N₂O₂S).

### Synthesis of N-(4-(3,5-dimethylphenyl)thiazol-2-yl)-4-methoxybenzamide (Compound 305)

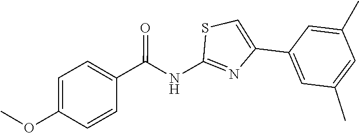

Compound N-(4-(3,5-dimethylphenyl)thiazol-2-yl)-4-methoxybenzamide was prepared from (3,5-dimethylphenyl)boronic acid following a procedure similar to that described for the synthesis of N-(4-(2,4-dichlorophenyl)thiazol-2-yl)-4-methoxybenzamide, and was isolated as an off-white solid.

Yield 51 mg (39%). ¹H NMR (400 MHz, DMSO) δ 12.55 (s, 1H), 8.16 (d, J=8.8 Hz, 2H), 7.60 (m, 3H), 7.10 (d, J=8.8 Hz, 2H), 6.99 (s, 1H), 3.88 (s, 3H), 2.34 (s, 6H). m/z: [ESI⁺] 339 (M+H)⁺, (C₁₉H₁₈N₂O₂S).

### Synthesis of N-(4-(3,5-dichlorophenyl)thiazol-2-yl)-4-methoxybenzamide (Compound 315)

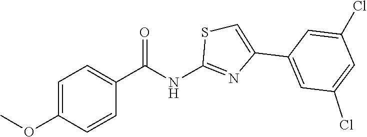 [SMILES: COc1ccc(C(=O)Nc2nc(-c3cc(Cl)cc(Cl)c3)cs2)cc1]

Compound N-(4-(3,5-dichlorophenyl)thiazol-2-yl)-4-methoxybenzamide was prepared from (3,5-dichlorophenyl)boronic acid following a procedure similar to that described for the synthesis of N-(4-(2,4-dichlorophenyl)thiazol-2-yl)-4-methoxybenzamide, and was isolated as an off-white solid.

Yield 43 mg (18%). ¹H NMR (400 MHz, DMSO) δ 12.61 (br s, 1H), 8.16 (d, J=8.9 Hz, 2H), 8.04 (d, J=2.0 Hz, 2H), 7.98 (s, 1H), 7.58 (dd, J=1.9, 1.9 Hz, 1H), 7.11 (d, J=8.9 Hz, 2H), 3.88 (s, 3H). m/z: [ESI⁺] 379 (M+H)⁺, (C₁₇H₁₂Cl₂N₂O₂S).

### Synthesis of N-(4-(2,4-dichlorophenyl)thiazol-2-yl)benzamide (Compound 302)

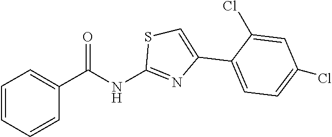 [SMILES: O=C(Nc1nc(-c2ccc(Cl)cc2Cl)cs1)c1ccccc1]

To a solution of 4-(2,4-dichlorophenyl)thiazol-2-amine (130 mg, 0.530 mmol) and benzoic acid (130 mg, 1.06 mmol) in anhydrous DCM (3 mL) was added triethylamine (0.44 mL, 3.18 mmol) followed by a solution of T3P (50% in ethyl acetate, 0.95 mL, 3.18 mmol). The reaction mixture was heated at 40° C. for 5 hours. After cooling to room temperature, the mixture was partitioned between DCM (10 mL) and saturated aqueous sodium hydrogen carbonate (10 mL). The aqueous layer was extracted with DCM (2×10 mL). The combined organic extracts were dried (MgSO₄), filtered and concentrated under reduced pressure. The crude product was purified by preparative HPLC to afford N-(4-(2,4-dichlorophenyl)thiazol-2-yl)benzamide as an off-white solid.

Yield 31 mg (17%). ¹H NMR (400 MHz, DMSO) δ 12.85 (s, 1H), 8.15 (d, J=7.6 Hz, 2H), 7.97 (d, J=8.6 Hz, 1H), 7.76 (s, 2H), 7.67 (dd, J=7.5, 7.5 Hz, 1H), 7.61-7.54 (m, 3H). m/z: [ESI⁺] 349 (M+H)⁺, (C₁₆H₁₀C₁₂N₂OS).

### Synthesis of 4-chloro-N-(4-(2,4-dichlorophenyl)thiazol-2-yl)benzamide (Compound 303)

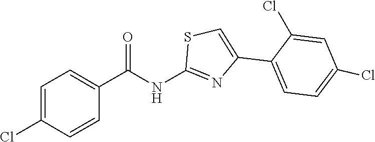 [SMILES: O=C(Nc1nc(-c2ccc(Cl)cc2Cl)cs1)c1ccc(Cl)cc1]

To a solution of 4-(2,4-dichlorophenyl)thiazol-2-amine (130 mg, 0.530 mmol) in anhydrous DCM (3 mL) was added 4-chlorobenzoyl chloride (0.1 mL, 0.796 mmol) followed by DMAP (65 mg, 0.530 mmol). The reaction mixture was stirred at room temperature for 5 hours. The reaction mixture was partitioned between DCM (10 mL) and saturated aqueous sodium hydrogen carbonate (10 mL). The aqueous layer was extracted with DCM) (2×10 mL). The combined organic extracts were dried (MgSO₄), filtered and concentrated under reduced pressure. The crude product was purified by preparative HPLC to afford 4-chloro-N-(4-(2,4-dichlorophenyl)thiazol-2-yl)benzamide as an off-white solid.

Yield 11 mg (5%). ¹H NMR (400 MHz, DMSO) δ 12.96-12.93 (br s, 1H), 8.16 (d, J=8.6 Hz, 2H), 7.96 (d, J=8.3 Hz, 1H), 7.77-7.72 (m, 2H), 7.64 (d, J=8.3 Hz, 2H), 7.57 (dd, J=1.9, 8.5 Hz, 1H). m/z: [ESI⁺] 383 (M+H)⁺, (C₁₆H₉C₁₃N₂OS).

### Synthesis of N-(4-(2,4-dichlorophenyl)thiazol-2-yl)-4-methylbenzamide (Compound 307)

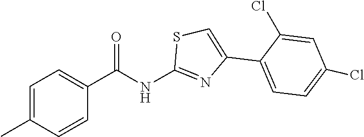 [SMILES: Cc1ccc(C(=O)Nc2nc(-c3ccc(Cl)cc3Cl)cs2)cc1]

Compound N-(4-(2,4-dichlorophenyl)thiazol-2-yl)-4-methylbenzamide was prepared from 4-methylbenzoic acid following a procedure similar to that described for the synthesis of N-(4-(2,4-dichlorophenyl)thiazol-2-yl)benzamide, and was isolated as a brown solid.

Yield 92 mg (52%). ¹H NMR (400 MHz, DMSO) δ 12.76 (s, 1H), 8.06 (d, J=8.2 Hz, 2H), 7.97 (d, J=8.4 Hz, 1H), 7.76-7.74 (m, 2H), 7.57 (dd, J=2.1, 8.5 Hz, 1H), 7.38 (d, J=8.0 Hz, 2H), 2.42 (s, 3H). m/z: [ESI⁺]363 (M+H)⁺, (C₇H₁₂Cl₂N₂OS).

### Synthesis of N-(4-(2,4-dichlorophenyl)thiazol-2-yl)-4-fluorobenzamide (Compound 308)

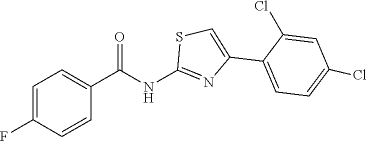 [SMILES: O=C(Nc1nc(-c2ccc(Cl)cc2Cl)cs1)c1ccc(F)cc1]

Compound N-(4-(2,4-dichlorophenyl)thiazol-2-yl)-4-fluorobenzamide was prepared from 4-fluorobenzoic acid following a procedure similar to that described for the synthesis of N-(4-(2,4-dichlorophenyl)thiazol-2-yl)benzamide, and was isolated as an off-white solid.

Yield 14 mg (8%). ¹H NMR (400 MHz, DMSO) δ 12.88 (br s, 1H), 8.23 (dd, J=5.3, 8.6 Hz, 2H), 7.96 (d, J=8.6 Hz, 1H), 7.76 (s, 2H), 7.57 (dd, J=2.0, 8.6 Hz, 1H), 7.41 (dd, J=8.8, 8.8 Hz, 2H). m/z: [ESI⁺] 367 (M+H)⁺, (C₁₆H₉Cl₂FN₂OS).

### Synthesis of N-(4-(2,4-dichlorophenyl)thiazol-2-yl)tetrahydro-2H-pyran-4-carboxamide (Compound 306)

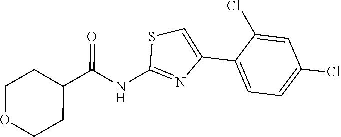 [SMILES: O=C(Nc1nc(-c2ccc(Cl)cc2Cl)cs1)C1CCOCC1]

Compound N-(4-(2,4-dichlorophenyl)thiazol-2-yl)tetrahydro-2H-pyran-4-carboxamide was prepared from tetrahydro-2H-pyran-4-carboxylic acid following a procedure similar to that described for the synthesis of N-(4-(2,4-dichlorophenyl)thiazol-2-yl)benzamide, and was isolated as a beige solid.

Yield 11 mg (6%). ¹H NMR (400 MHz, DMSO) δ 12.34 (br s, 1H), 7.88 (d, J=8.3 Hz, 1H), 7.74 (d, J=1.8 Hz, 1H), 7.67 (s, 1H), 7.54 (dd, J=1.9, 8.5 Hz, 1H), 3.93 (d, J=10.6 Hz, 2H), 3.41-3.35 (m, 2H), 2.83-2.74 (m, 1H), 1.81-1.65 (m, 4H). m/z: [ESI⁺] 357 (M+H)⁺, (C₁₅H₁₄Cl₂N₂O₂S).

### Synthesis of 4-methoxy-N-[4-(3-pyridyl)thiazol-2-yl]benzamide (Compound 316)

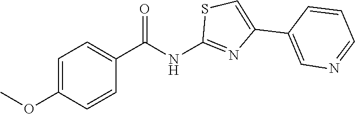 [SMILES: COc1ccc(C(=O)Nc2nc(-c3cccnc3)cs2)cc1]

To a degassed mixture of N-(4-bromothiazol-2-yl)-4-methoxybenzamide (100 mg, 0.319 mmol) and pyridine-3-ylboronic acid (78 mg, 0.639 mmol) in 1,4-dioxane (4 mL) was added at room temperature [1,1′-bis(diphenylphosphino)ferrocene] dichloropalladium(II), complex with dichloromethane (26 mg, 0.032 mmol), and a solution of sodium carbonate (102 mg, 0.96 mmol) in water (0.5 mL). The reaction mixture was heated at 120° C. in a microwave for 1 hour. After cooling to room temperature, the mixture was partitioned between ethyl acetate (15 mL) and brine (10 mL). The layers were separated, and the aqueous phase was extracted with ethyl acetate (2×15 mL). The combined organic extracts were dried (MgSO₄), filtered and evaporated. The crude product was purified by preparative HPLC to afford 4-methoxy-N-[4-(3-pyridyl)thiazol-2-yl]benzamide as an off-white solid.

Yield 22 mg (22%). ¹H NMR (400 MHz, DMSO) δ 12.68 (br s, 1H), 9.19 (s, 1H), 8.55 (d, J=3.6 Hz, 1H), 8.29 (d, J=7.9 Hz, 1H), 8.16 (d, J=8.7 Hz, 2H), 7.84 (s, 1H), 7.49 (dd, J=4.8, 7.9 Hz, 1H), 7.10 (d, J=8.7 Hz, 2H), 3.87 (s, 3H). m/z: [ESI⁺] 312 (M+H)⁺, (C₁₆H₁₃N₃O₂S).

### Synthesis of N-[4-(2-chloro-4-fluoro-phenyl)thiazol-2-yl]-4-methoxy-benzamide (Compound 321)

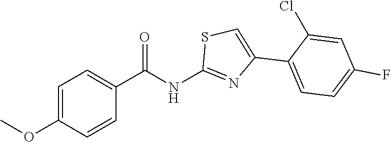 [SMILES: COc1ccc(C(=O)Nc2nc(-c3ccc(F)cc3Cl)cs2)cc1]

Compound N-[4-(2-chloro-4-fluoro-phenyl)thiazol-2-yl]-4-methoxy-benzamide was prepared from (2-chloro-4-fluorophenyl)boronic acid following a similar procedure to that described for the synthesis of 4-methoxy-N-[4-(3-pyridyl)thiazol-2-yl]benzamide, and was isolated as an off-white solid.

Yield 47 mg (34%). ¹H NMR (400 MHz, DMSO) δ 12.65 (br s, 1H), 8.15 (d, J=8.9 Hz, 2H), 7.95 (dd, J=6.4, 8.8 Hz, 1H), 7.64 (s, 1H), 7.58 (dd, J=2.6, 8.8 Hz, 1H), 7.39-7.33 (m, 1H), 7.10 (d, J=8.9 Hz, 2H), 3.87 (s, 3H). m/z: [ESI⁺] 363 (M+H)⁺, (C₁₇H₁₂ClFN₂O₂S).

### Synthesis of N-[4-(2,4-difluorophenyl)thiazol-2-yl]-4-methoxy-benzamide (Compound 325)

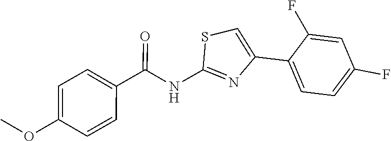 [SMILES: COc1ccc(C(=O)Nc2nc(-c3ccc(F)cc3F)cs2)cc1]

Compound N-[4-(2,4-difluorophenyl)thiazol-2-yl]-4-methoxy-benzamide was prepared from (2,4-difluorophenyl)boronic acid following a similar procedure to that described for the synthesis of 4-methoxy-N-[4-(3-pyridyl)thiazol-2-yl]benzamide, and was isolated as an off-white solid.

Yield 9 mg (7%). ¹H NMR (400 MHz, DMSO) δ 12.65 (br s, 1H), 8.18-8.12 (m, 1H), 8.16 (d, J=9.0 Hz, 2H), 7.54 (d, J=2.6 Hz, 1H), 7.44-7.37 (m, 1H), 7.27-7.21 (m, 1H), 7.10 (d, J=9.0 Hz, 2H), 3.87 (s, 3H). m/z: [ESI⁺] 347 (M+H)⁺, (C₁₇H₁₂F₂N₂O₂S).

### Synthesis of 4-methoxy-N-[4-(4-methoxy-3-pyridyl)thiazol-2-yl]benzamide (Compound 326)

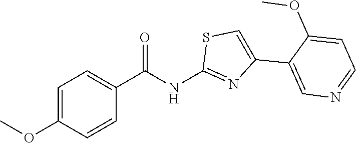 [SMILES: COc1ccc(C(=O)Nc2nc(-c3cnccc3OC)cs2)cc1]

To a degassed mixture of N-(4-bromothiazol-2-yl)-4-methoxybenzamide (120 mg, 0.383 mmol) and (4-methoxypyridin-3-yl)boronic acid (117 mg, 0.766 mmol) in 1,4-dioxane (4 mL) was added at room temperature bis(di-tert-butyl(4-dimethylaminophenyl)phosphine)dichloropalladium(II) (27 mg, 0.038 mmol) and a solution of cesium carbonate (375 mg, 1.15 mmol) in water (1 mL). The reaction mixture was heated at 120° C. in a microwave for 1 hour. Further (4-methoxypyridin-3-yl)boronic acid (117 mg, 0.766 mmol) and bis(di-tert-butyl(4-dimethylaminophenyl)phosphine)dichloropalladium(II) (27 mg, 0.038 mmol) was added and the reaction heated at 120° C. in a microwave for 1.5 hours. Further (4-methoxypyridin-3-yl)boronic acid (117 mg, 0.766 mmol) and bis(di-tert-butyl(4-dimethylaminophenyl)phosphine)dichloropalladium(II) (27 mg, 0.038 mmol) was added and the reaction heated at 120° C. in a microwave for another 1.5 hours. After cooling to room temperature, the mixture was partitioned between ethyl acetate (15 mL) and brine (10 mL). The layers were separated, and the aqueous phase was extracted with ethyl acetate (2×15 mL). The combined organic extracts were dried (MgSO₄), filtered and evaporated. The crude product was purified by preparative HPLC to afford 4-methoxy-N-[4-(4-methoxy-3-pyridyl)thiazol-2-yl]benzamide as an off-white solid.

Yield 30 mg (23%). ¹H NMR (400 MHz, DMSO) δ 12.53 (br s, 1H), 9.10 (s, 1H), 8.34 (d, J=5.8 Hz, 111), 8.07 (d, J=9.0 Hz, 2H), 7.66 (s, 1H), 7.12 (d, J=5.8 Hz, 1H), 7.02 (d, J=9.0 Hz, 2H), 3.94 (s, 3H), 3.80 (s, 3H). m/z: [ESI⁺] 342 (M+H)⁺, (C₁₇H₁₅N₃O₃S).

### Synthesis of 4-methoxy-N-[4-[2-(2-methoxyethoxy)phenyl]thiazol-2-yl]benzamide (Compound 328)

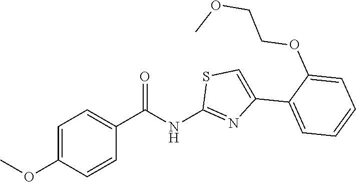 [SMILES: COCCOc1ccccc1-c1csc(NC(=O)c2ccc(OC)cc2)n1]

To a degassed mixture of N-(4-bromothiazol-2-yl)-4-methoxybenzamide (120 mg, 0.383 mmol) and (2-(2-methoxyethoxy)phenyl)boronic acid (150 mg, 0.766 mmol) in 1,4-dioxane (4 mL) was added at room temperature bis(di-tert-butyl(4-dimethylaminophenyl)phosphine)dichloropalladium(II) (27 mg, 0.038 mmol) and a solution of cesium carbonate (375 mg, 1.15 mmol) in water (0.5 mL). The reaction mixture was heated at 120° C. in a microwave for 1 hour. After cooling to room temperature, the mixture was partitioned between ethyl acetate (15 mL) and brine (10 mL). The layers were separated, and the aqueous phase was extracted with ethyl acetate (2×15 mL). The combined organic extracts were dried (MgSO₄), filtered and evaporated. The crude product was purified by preparative HPLC to afford 4-methoxy-N-[4-[2-(2-methoxyethoxy)phenyl]thiazol-2-yl]benzamide as an off-white solid.

Yield 85 mg (58%). ¹H NMR (400 MHz, DMSO) δ 12.55 (s, 1H), 8.26 (d, J=7.6 Hz, 1H), 8.20 (d, J=9.0 Hz, 2H), 7.85 (s, 1H), 7.38-7.33 (m, 1H), 7.19 (d, J=7.6 Hz, 1H), 7.16-7.10 (m, 3H), 4.33-4.29 (m, 2H), 3.92 (s, 3H), 3.88-3.84 (m, 2H), 3.43 (s, 3H). m/z: [ESI⁺] 385 (M+H)⁺, (C₂₀H₂₀N₂O₄S).

### Synthesis of 4-methoxy-N-[4-(1-methylpyrazol-4-yl)thiazol-2-yl]benzamide (Compound 331)

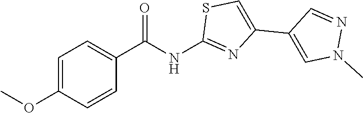

Compound 4-methoxy-N-[4-(1-methylpyrazol-4-yl)thiazol-2-yl]benzamide was prepared from 1-methyl-4-(4,4,5,5-tetramethyl-1,3,2-dioxaborolan-2-yl)-1H-pyrazole following a similar procedure to that described for the synthesis of 4-methoxy-N-[4-[2-(2-methoxyethoxy)phenyl]thiazol-2-yl]benzamide, except that it was purified by column chromatography on silica gel (0-50% ethyl acetate in cyclohexane), and was isolated as a beige solid.

Yield 51 mg (42%). ¹H NMR (400 MHz, DMSO) δ 12.47 (s, 1H), 8.06 (d, J=9.0 Hz, 2H), 7.93 (s, 1H), 7.73 (s, 111), 7.14 (s, 1H), 7.01 (d, J=9.0 Hz, 2H), 3.81 (s, 3H), 3.79 (s, 3H). m/z: [ESI⁺] 315 (M+H)⁺, (C₁₅H₁₄N₄O₂S).

### Synthesis of 4-methoxy-N-[4-(2-methoxy-3-pyridyl)thiazol-2-yl]benzamide (Compound 332)

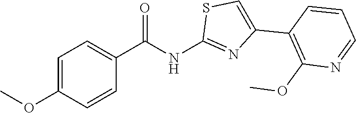 [SMILES: COc1ccc(C(=O)Nc2nc(-c3cccnc3OC)cs2)cc1]

Compound 4-methoxy-N-[4-(2-methoxy-3-pyridyl)thiazol-2-yl]benzamide was prepared from 2-methoxy-3-(4,4,5,5-tetramethyl-1,3,2-dioxaborolan-2-yl)pyridine following a similar procedure to that described for the synthesis of 4-methoxy-N-[4-[2-(2-methoxyethoxy)phenyl]thiazol-2-yl]benzamide, except that it was purified by column chromatography on silica gel (0-15% ethyl acetate in cyclohexane), and was isolated as an off-white solid.

Yield 91 mg (70%). ¹H NMR (400 MHz, DMSO) δ 12.49 (s, 1H), 8.39 (dd, J=2.0, 7.6 Hz, 1H), 8.11-8.06 (m, 3H), 7.72 (s, 1H), 7.07 (dd, J=4.8, 7.6 Hz, 1H), 7.02 (d, J=9.0 Hz, 2H), 3.97 (s, 3H), 3.79 (s, 311). m/z: [ESI⁺] 342 (M+H)⁺, (C₁₇H₁₅N₃O₃S).

### Synthesis of 4-methoxy-N-[4-(4-methyl-3-pyridyl)thiazol-2-yl]benzamide (Compound 333)

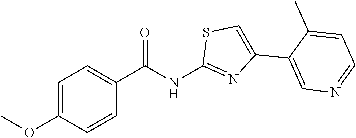 [SMILES: COc1ccc(C(=O)Nc2nc(-c3cnccc3C)cs2)cc1]

Compound 4-methoxy-N-[4-(4-methyl-3-pyridyl)thiazol-2-yl]benzamide was prepared from 4-methyl-3-(4,4,5,5-tetramethyl-1,3,2-dioxaborolan-2-yl)pyridine following a similar procedure to that described for the synthesis of 4-methoxy-N-[4-[2-(2-methoxyethoxy)phenyl]thiazol-2-yl]benzamide, except that it was purified by column chromatography on silica gel (0-50% ethyl acetate in cyclohexane), and was isolated as a yellow solid.

Yield 52 mg (42%). ¹H NMR (400 MHz, DMSO) δ 12.52 (s, 1H), 8.71 (s, 1H), 8.34 (d, J=5.1 Hz, 1H), 8.06 (d, J=9.0 Hz, 2H), 7.41 (s, 1H), 7.26 (d, J=5.1 Hz, 1H), 7.02 (d, J=9.0 Hz, 2H), 3.80 (s, 3H), 2.43 (s, 3H). m/z: [ESI⁺] 326 (M+H)⁺, (C₁₇H₁₅N₃O₂S).

### Synthesis of 4-methoxy-N-[4-(4-methylpyrimidin-5-yl)thiazol-2-yl]benzamide (Compound 335)

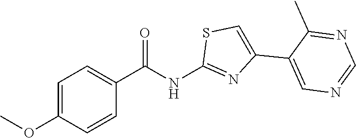 [SMILES: COc1ccc(C(=O)Nc2nc(-c3cncnc3C)cs2)cc1]

To a degassed mixture of 5-bromo-4-methylpyrimidine (83 mg, 0.479 mmol), bis(pinacolato)diboron (122 mg, 0.479 mmol) and potassium acetate (165 mg, 1.68 mmol) in 1,4-dioxane (4 ml.) was added at room temperature [1,1′-bis(diphenylphosphino)ferrocene]dichloropalladium(II) complex with dichloromethane (39 mg, 0.048 mmol) and the reaction was heated at 105° C. for 4 hours under a nitrogen atmosphere. After cooling to room temperature, N-(4-bromothiazol-2-yl)-4-methoxybenzamide (60 mg, 0.192 mmol) and cesium carbonate (312 mg, 0.958 mmol) in water (0.5 mL) were added and the mixture was heated at 120° C. in a microwave for 1 hour. After cooling to room temperature, the mixture was partitioned between ethyl acetate (15 mL) and brine (10 mL). The layers were separated, and the aqueous phase was extracted with ethyl acetate (2×15 mL). The combined organic extracts were dried (MgSO₄), filtered and evaporated. The crude product was purified by column chromatography on silica gel (0-100% ethyl acetate in cyclohexane followed by 0-10% methanol in ethyl acetate) to provide and oily solid that was further purified by preparative HPLC to afford 4-methoxy-N-[4-(4-methylpyrimidin-5-yl)thiazol-2-yl]benzamide as an off-white solid.

Yield 8 mg (5%). ¹H NMR (400 MHz, DMSO) δ 12.70 (br s, 1H), 9.08 (s, 1H), 9.02 (s, 1H), 8.19 (d, J=9.1 Hz, 2H), 7.66 (s, 1H), 7.14 (d, J=9.1 Hz, 2H), 3.91 (s, 3H), 2.75 (s, 3H). m/z: [ESI⁺] 327 (M+H)⁺, (C₁₆H₁₄N₄O₂S).

### Synthesis of 4-methoxy-N-(4-pyrimidin-5-ylthiazol-2-yl)benzamide (Compound 338)

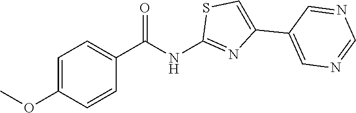 [SMILES: COc1ccc(C(=O)Nc2nc(-c3cncnc3)cs2)cc1]

Compound 4-methoxy-N-(4-pyrimidin-5-ylthiazol-2-yl)benzamide was prepared from pyrimidin-5-ylboronic acid following a similar procedure to that described for the synthesis of 4-methoxy-N-[4-[2-(2-methoxyethoxy)phenyl]thiazol-2-yl]benzamide, except that it was purified by column chromatography on silica gel (0-50% ethyl acetate in cyclohexane) and by preparative HPLC, and was isolated as a colourless solid.

Yield 2 mg (2%). ¹H NMR (400 MHz, DMSO) δ 12.75 (br s, 1H), 9.33 (s, 2H), 9.16 (s, 1H), 8.15 (d, J=8.9 Hz, 2H), 7.96 (s, 1H), 7.09 (d, J=8.9 Hz, 2H), 3.87 (s, 3H). m/z: [ESI⁺] 313 (M+H)⁺, (C₁₅H₁₂N₄O₂S).

### Synthesis of N-[4-(2,3-dihydro-1,4-benzodioxin-5-yl)thiazol-2-yl]-4-methoxy-benzamide (Compound 337)

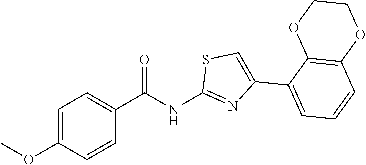 [SMILES: COc1ccc(C(=O)Nc2nc(-c3cccc4c3OCCO4)cs2)cc1]

Compound N-[4-(2,3-dihydro-1,4-benzodioxin-5-yl)thiazol-2-yl]-4-methoxy-benzamide was prepared from (2,3-dihydrobenzo[b][1,4]dioxin-5-yl)boronic acid following a similar procedure to that described for the synthesis of 4-methoxy-N-[4-[2-(2-methoxyethoxy)phenyl]thiazol-2-yl]benzamide, except that it was purified by column chromatography on silica gel (0-15% ethyl acetate in cyclohexane), and was isolated as an off-white solid.

Yield 122 mg (86%). ¹H NMR (400 MHz, DMSO) δ 12.57 (s, 1H), 8.19 (d, J=8.9 Hz, 2H), 7.76-7.70 (m, 2H), 7.14 (d, J=8.9 Hz, 2H), 6.96 (dd, J=7.8, 7.8 Hz, 1H), 6.90 (dd, J=1.8, 7.8 Hz, 1H), 4.48-4.43 (m, 2H), 4.39-4.34 (m, 2H), 3.91 (s, 3H). m/z: [ESI⁺] 369 (M+H)⁺, (C₁₉H₁₆N₂O₄S).

### Synthesis of N-[4-(2-fluorophenyl)thiazol-2-yl]-4-methoxy-benzamide (Compound 336)

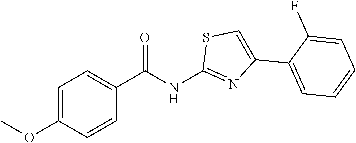 [SMILES: COc1ccc(C(=O)Nc2nc(-c3ccccc3F)cs2)cc1]

To a degassed mixture of N-(4-bromothiazol-2-yl)-4-methoxybenzamide (300 mg, 0.958 mmol) and (2-fluorophenyl)boronic acid (268 mg, 1.92 mmol) in 1,4-dioxane (8 mL) was added at room temperature bis(di-tert-butyl(4-dimethylaminophenyl)phosphine)dichloropalladium(II) (68 mg, 0.096 mmol) and a solution of cesium carbonate (936 mg, 2.87 mmol) in water (1 mL). The reaction mixture was heated at 85° C. for 18 hours. After cooling to room temperature, the mixture was partitioned between ethyl acetate (15 mL) and brine (10 mL). The layers were separated, and the aqueous phase was extracted with ethyl acetate (2×15 mL). The combined organic extracts were dried (MgSO₄), filtered and evaporated. The crude product was purified by column chromatography on silica gel (0-50% ethyl acetate in cyclohexane) to furnish a brown solid (359 mg). A part of this material (50 mg) was purified by preparative HPLC to afford N-[4-(2-fluorophenyl)thiazol-2-yl]-4-methoxy-benzamide as a colourless solid.

Yield 34 mg (11%). ¹H NMR (400 MHz, DMSO) δ 12.65 (br s, 1H), 8.19-8.16 (m, 1H), 8.18 (d, J=9.0 Hz, 2H), 7.62 (d, J=2.5 Hz, 1H), 7.48-7.41 (m, 1H), 7.40-7.33 (m, 2H), 7.13 (d, J=9.0 Hz, 2H), 3.90 (s, 3H). m/z: [ESI⁺] 329 (M+H)⁺, (C₁₇H₁₃FN₂O₂S).

### Synthesis of 4-methoxy-N-[4-(2-methyl-3-pyridyl)thiazol-2-yl]benzamide (Compound 343)

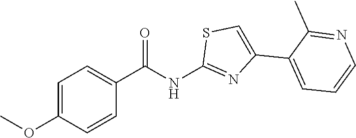 [SMILES: COc1ccc(C(=O)Nc2nc(-c3cccnc3C)cs2)cc1]

Compound 4-methoxy-N-[4-(2-methyl-3-pyridyl)thiazol-2-yl]benzamide was prepared from (2-methylpyridin-3-yl)boronic acid following a similar procedure to that described for the synthesis of 4-methoxy-N-[4-[2-(2-methoxyethoxy)phenyl]thiazol-2-yl]benzamide, except that it was purified by column chromatography on silica gel (0-60% ethyl acetate in cyclohexane) after preparative HPLC purification, and was isolated as a colourless solid.

Yield 20 mg (16%). ¹H NMR (400 MHz, DMSO) δ 12.65 (br s, 1H), 8.49 (dd, J=1.8, 4.8 Hz, 1H), 8.15 (d, J=9.0 Hz, 2H), 8.05 (dd, J=1.8, 7.8 Hz, 1H), 7.49 (s, 1H), 7.38 (dd, J=4.8, 7.8 Hz, 1H), 7.12 (d, J=9.0 Hz, 2H), 3.90 (s, 3H), 2.71 (s, 3H). m/z: [ESI⁺] 326 (M+H)⁺, (C₁₇H₁₅N₃O₂S).

### Synthesis of 6-methyl-N-[4-(1-methylpyrazol-4-yl)thiazol-2-yl]pyridine-3-carboxamide (Compound 317)

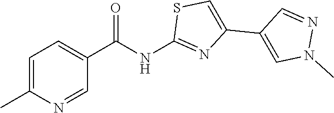

To a degassed mixture of N-(4-bromothiazol-2-yl)-6-methylnicotinamide (95 mg, 0.319 mmol) and 1-methyl-4-(4,4,5,5-tetramethyl-1,3,2-dioxaborolan-2-yl)-1H-pyrazole (133 mg, 0.639 mmol) in 1,4-dioxane (4 mL) was added at room temperature [1,1′-bis(diphenylphosphino)ferrocene]dichloropalladium(II), complex with dichloromethane (26 mg, 0.032 mmol), and a solution of sodium carbonate (102 mg, 0.96 mmol) in water (0.5 mL). The reaction mixture was heated at 120° C. in a microwave for 1 hour. After cooling to room temperature, the mixture was partitioned between ethyl acetate (15 mL) and brine (10 mL). The layers were separated, and the aqueous phase was extracted with ethyl acetate (2×15 mL). The combined organic extracts were dried (MgSO₄), filtered and evaporated. The crude product was purified by preparative HPLC to afford N-[4-(1-methylpyrazol-4-yl)thiazol-2-yl]pyridine-3-carboxamide as an off-white solid.

Yield 20 mg (21%). ¹H NMR (400 MHz, DMSO) δ 12.94 (br s, 1H), 9.14 (d, J=2.0 Hz, 1H), 8.35 (dd, J=2.0, 8.2 Hz, 1H), 7.61 (s, 1H), 7.48-7.45 (m, 2H), 6.63 (d, J=2.0 Hz, 1H), 4.11 (s, 3H), 2.58 (s, 3H). m/z: [ESI⁺] 300 (M+H)⁺, (C₁₄H₁₃N₅OS).

### Synthesis of N-[4-(2-methoxy-3-pyridyl)thiazol-2-yl]-6-methyl-pyridine-3-carboxamide (Compound 319)

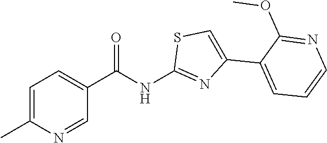 [SMILES: COc1ncccc1-c1csc(NC(=O)c2ccc(C)nc2)n1]

Compound N-[4-(2-methoxy-3-pyridyl)thiazol-2-yl]-6-methyl-pyridine-3-carboxamide was prepared from 2-methoxy-3-(4,4,5,5-tetramethyl-1,3,2-dioxaborolan-2-yl)pyridine following a similar procedure to that described for the synthesis of N-[4-(1-methylpyrazol-4-yl)thiazol-2-yl]pyridine-3-carboxamide, and was isolated as an off-white solid.

Yield 27 mg (26%). ¹H NMR (400 MHz, DMSO) δ 9.18 (s, 1H), 8.51 (d, J=7.1 Hz, 1H), 8.39 (d, J=8.1 Hz, 1H), 8.21 (d, J=3.5 Hz, 1H), 7.86 (s, 1H), 7.49 (d, J=8.1 Hz, 1H), 7.22-7.17 (m, 1H), 4.09 (s, 3H), 2.61 (s, 3H). NH proton obscured. m/z: [ESI⁺] 327 (M+H)⁺, (C₁₆H₁₄N₄O₂S).

### Synthesis of 6-methyl-N-[4-(3-pyridyl)thiazol-2-yl]pyridine-3-carboxamide (Compound 323)

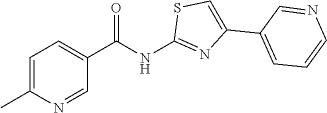 [SMILES: Cc1ccc(C(=O)Nc2nc(-c3cccnc3)cs2)cn1]

Compound N-[4-(3-pyridyl)thiazol-2-yl]pyridine-3-carboxamide was prepared from pyridin-3-ylboronic acid following a similar procedure to that described for the synthesis of N-[4-(1-methylpyrazol-4-yl)thiazol-2-yl]pyridine-3-carboxamide, except that it was triturated with diethyl ether and petrol ether after purification, and was isolated as an off-white solid.

Yield 5 mg (5%). ¹H NMR (400 MHz, DMSO) δ 12.90 (br s, 1H), 9.09 (dd, J=2.0, 13.3 Hz, 2H), 8.47 (dd, J=2.0, 4.7 Hz, 1H), 8.28 (dd, J=2.0, 8.1 Hz, 1H), 8.23-8.19 (m, 1H), 7.79 (s, 1H), 7.43-7.39 (m, 1H), 7.37 (d, J=8.1 Hz, 1H), 2.50 (s, 3H). m/z: [ESI⁺] 297 (M+H)⁺, (C₁₅H₁₂N₄OS).

### Synthesis of 6-methyl-N-(4-pyrimidin-5-ylthiazol-2-yl)pyridine-3-carboxamide (Compound 340)

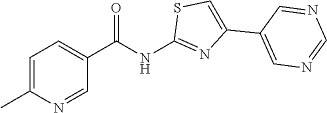 [SMILES: Cc1ccc(C(=O)Nc2nc(-c3cncnc3)cs2)cn1]

To a degassed mixture of N-(4-bromothiazol-2-yl)-6-methylnicotinamide (114 mg, 0.383 mmol) and pyrimidin-5-ylboronic acid (95 mg, 0.766 mmol) in 1,4-dioxane (4 mL) was added at room temperature bis(di-tert-butyl(4-dimethylaminophenyl)phosphine)dichloropalladium(II) (27 mg, 0.038 mmol) and a solution of cesium carbonate (375 mg, 1.15 mmol) in water (0.5 mL). The reaction mixture was degassed (N₂ stream) for 5 minutes, sealed and heated at 120° C. in a microwave for 1 hour. The reaction was re-charged twice with pyrimidin-5-ylboronic acid (95 mg, 0.766 mmol) and bis(di-tert-butyl(4-dimethylaminophenyl)phosphine)dichloropalladium(II) (27 mg, 0.038 mmol) and heated at 120° C. in a microwave for 1 hour and 3 hours respectively. After cooling to room temperature, the mixture was partitioned between ethyl acetate (15 mL) and brine (10 mL). The layers were separated, and the aqueous phase was extracted with ethyl acetate (2×15 mL). The combined organic extracts were dried (MgSO₄), filtered and evaporated. The crude product was purified by preparative HPLC to afford 6-methyl-N-(4-pyrimidin-5-ylthiazol-2-yl)pyridine-3-carboxamide as an off-white solid.

Yield 8 mg (7%). ¹H NMR (400 MHz, DMSO) δ 9.26 (s, 2H), 9.09 (s, 1H), 9.06 (d, J=2.4 Hz, 1H), 8.26 (dd, J=2.4, 8.2 Hz, 1H), 7.95 (s, 1H), 7.39 (d, J=8.2 Hz, 1H), 2.51 (s, 3H). NH proton obscured. m/z: [ESI⁺]297 (M+H)⁺, (C₁₄H₁₁N₅OS).

### Synthesis of N-[4-[2-(2-methoxyethoxy)phenyl]thiazol-2-yl]-6-methyl-pyridine-3-carboxamide (Compound 341)

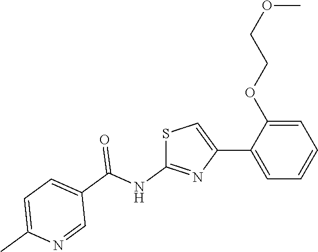 [SMILES: COCCOc1ccccc1-c1csc(NC(=O)c2ccc(C)nc2)n1]

Compound N-[4-[2-(2-methoxyethoxy)phenyl]thiazol-2-yl]-6-methyl-pyridine-3-carboxamide was prepared from (2-(2-methoxyethoxy)phenyl)boronic acid following a similar procedure to that described for the synthesis of 6-methyl-N-(4-pyrimidin-5-ylthiazol-2-yl)pyridine-3-carboxamide, and was isolated as an off-white solid.

Yield 38 mg (27%). ¹H NMR (400 MHz, DMSO) δ 12.83 (br s, 1H), 9.13 (d, J=2.0 Hz, 1H), 8.34 (dd, J=2.4, 8.2 Hz, 1H), 8.19 (dd, J=2.0, 7.8 Hz, 1H), 7.83 (s, 1H), 7.45 (d, J=8.2 Hz, 1H), 7.32 (dd, J=7.5, 8.2 Hz, 1H), 7.15 (d, J=7.8 Hz, 1H), 7.07 (dd, J=7.5, 7.5 Hz, 1H), 4.28-4.24 (m, 2H), 3.84-3.79 (m, 2H), 3.38 (s, 3H), 2.57 (s, 3H). m/z: [ESI⁺] 370 (M+H)⁺, (C₁₉H₁₉N₃O₃S). Synthesis of N-[4-(2,3-dihydro-1,4-benzodioxin-5-yl)thiazol-2-yl]-6-methyl-pyridine-3-carboxamide (Compound 342)

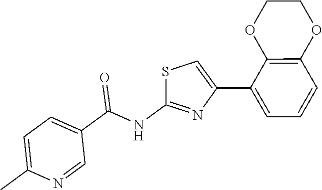 [SMILES: Cc1ccc(C(=O)Nc2nc(-c3cccc4c3OCCO4)cs2)cn1]

Compound N-[4-(2,3-dihydro-1,4-benzodioxin-5-yl)thiazol-2-yl]-6-methyl-pyridine-3-carboxamide was prepared from (2,3-dihydrobenzo[b][1,4]dioxin-5-yl)boronic acid following a similar procedure to that described for the synthesis of 6-methyl-N-(4-pyrimidin-5-ylthiazol-2-yl)pyridine-3-carboxamide, and was isolated as an off-white solid.

Yield 53 mg (39%). ¹H NMR (400 MHz, DMSO) δ 12.86 (br s, 1H), 9.14 (d, J=2.0 Hz, 1H), 8.36 (dd, J=2.4, 8.0 Hz, 1H), 7.72-7.67 (m, 2H), 7.45 (d, J=8.2 Hz, 1H), 6.93 (dd, J=7.8, 7.8 Hz, 1H), 6.86 (dd, J=2.0, 8.0 Hz, 1H), 4.44-4.40 (m, 2H), 4.34-4.31 (m, 2H), 2.58 (s, 3H). m/z: [ESI⁺] 354 (M+H)⁺, (C₁₈H₁₅N₃O₃S).

### Synthesis of 6-methyl-N-[4-(2-methyl-3-pyridyl)thiazol-2-yl]pyridine-3-carboxamide (Compound 344)

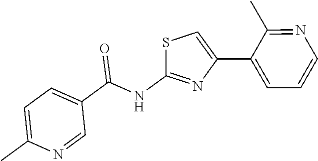 [SMILES: Cc1ccc(C(=O)Nc2nc(-c3cccnc3C)cs2)cn1]

Compound 6-methyl-N-[4-(2-methyl-3-pyridyl)thiazol-2-yl]pyridine-3-carboxamide was prepared from (2-methylpyridin-3-yl)boronic acid following a similar procedure to that described for the synthesis of 6-methyl-N-(4-pyrimidin-5-ylthiazol-2-yl)pyridine-3-carboxamide, except it was purified by column chromatography on silica gel (0-100% ethyl acetate in cyclohexane) prior to preparative HPLC purification, and was isolated as a colourless solid.

Yield 26 mg (22%). ¹H NMR (400 MHz, DMSO) δ 12.93 (s, 1H), 9.14 (d, J=2.0 Hz, 1H), 8.47 (dd, J=2.0, 4.8 Hz, 1H), 8.36 (dd, J=2.0, 8.2 Hz, 1H), 8.01 (dd, J=2.0, 7.8 Hz, 1H), 7.51 (s, 1H), 7.46 (d, J=8.2 Hz, 1H), 7.34 (dd, J=4.8, 7.8 Hz, 1H), 2.68 (s, 3H), 2.58 (s, 3H). m/z: [ESI⁺] 311 (M+H)⁺, (C₁₆H₁₄N₄OS).

### Synthesis of 6-methyl-N-[4-(4-methylpyrimidin-5-yl)thiazol-2-yl]pyridine-3-carboxamide (Compound 347)

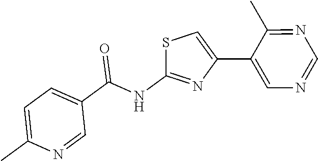 [SMILES: Cc1ccc(C(=O)Nc2nc(-c3cncnc3C)cs2)cn1]

To a degassed mixture of 5-bromo-4-methylpyrimidine (87 mg, 0.503 mmol), bis(pinacolato)diboron (128 mg, 0.503 mmol) and potassium acetate (173 mg, 1.76 mmol) in 1,4-dioxane (4 mL) was added at room temperature [1,1′-bis(diphenylphosphino)ferrocene]dichloropalladium(II) complex with dichloromethane (41 mg, 0.05 mmol) and the reaction was heated at 105° C. for 2 hours under a nitrogen atmosphere. After cooling to room temperature, N-(4-bromothiazol-2-yl)-6-methylnicotinamide (150 mg, 0.503 mmol) and cesium carbonate (328 mg, 1.01 mmol) in water (0.5 mL) were added and the mixture was heated at 120° C. in a microwave for 1 hour. After cooling to room temperature, the reaction mixture was diluted with ethyl acetate, filtered through Celite and the filtrate evaporated to dryness. The residue was purified by column chromatography on silica gel (0-100% ethyl acetate in cyclohexane followed by 0-10% methanol in ethyl acetate) to provide an orange solid that was further purified by preparative HPLC to afford 6-methyl-N-[4-(4-methylpyrimidin-5-yl)thiazol-2-yl]pyridine-3-carboxamide as an off-white solid.

Yield 8 mg (5%). ¹H NMR (400 MHz, DMSO) δ 12.99 (br s, 1H), 9.15 (d, J=2.4 Hz, 1H), 9.05 (s, 111), 8.99 (s, 1H), 8.36 (dd, J=2.4, 8.2 Hz, 1H), 7.70 (s, 1H), 7.47 (d, J=8.2 Hz, 1H), 2.72 (s, 3H), 2.59 (s, 3H). m/z: [ESI⁺] 312 (M+H)⁺, (C₁₅H₁₃N₅OS).

### Synthesis of 6-methyl-N-[4-(4-methyl-3-pyridyl)thiazol-2-yl]pyridine-3-carboxamide (Compound 348)

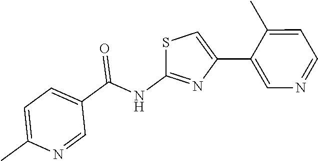 [SMILES: Cc1ccc(C(=O)Nc2nc(-c3cnccc3C)cs2)cn1]

Compound N-[4-(4-methyl-3-pyridyl)thiazol-2-yl]pyridine-3-carboxamide was prepared from 4-methyl-3-(4,4,5,5-tetramethyl-1,3,2-dioxaborolan-2-yl)pyridine following a similar procedure to that described for the synthesis of 6-methyl-N-(4-pyrimidin-5-ylthiazol-2-yl)pyridine-3-carboxamide, except it was purified by column chromatography on silica gel (0-100% ethyl acetate in cyclohexane followed by 0-10% methanol in ethyl acetate) prior to preparative HPLC purification, and was isolated as an off-white solid.

Yield 20 mg (8%). ¹H NMR (400 MHz, DMSO) δ 12.85 (s, 1H), 9.06 (d, J=2.4 Hz, 1H), 8.71 (s, 1H), 8.35 (d, J=5.0 Hz, 1H), 8.27 (dd, J=2.4, 8.1 Hz, 1H), 7.45 (s, 1H), 7.38 (d, J=8.1 Hz, 1H), 7.27 (d, J=5.0 Hz, 1H), 2.50 (s, 3H), 2.43 (s, 3H). m/z: [ESI⁺] 311 (M+H)⁺, (C₁₆H₁₄N₄OS).

### Synthesis of N-[4-[2-(azetidin-1-yl)phenyl]thiazol-2-yl]-6-methyl-pyridine-3-carboxamide (Compound 350)

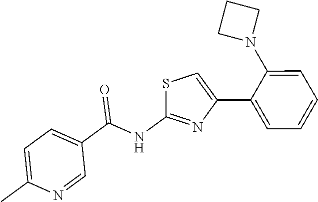 [SMILES: Cc1ccc(C(=O)Nc2nc(-c3ccccc3N3CCC3)cs2)cn1]

To a degassed mixture of 1-(2-bromophenyl)azetidine (150 mg, 0.707 mmol), tetrahydroxydiboron (190 mg, 2.12 mmol) and potassium acetate (208 mg, 2.12 mmol) in ethanol (3 mL) was added XPhos Pd G2 (56 mg, 0.071 mmol), XPhos (67 mg, 0.141 mmol) and ethylene glycol (39 μL, 0.71 mmol). The reaction mixture was heated at 80° C. for 2 hours under a nitrogen atmosphere. After cooling to room temperature, N-(4-bromothiazol-2-yl)-6-methylnicotinamide (169 mg, 0.566 mmol) and cesium carbonate (691 mg, 2.12 mmol) were added and the mixture was heated at 80° C. for 24 hours. After cooling to room temperature, the reaction mixture was filtered through Celite and the filtrate was washed with brine. The layers were separated, and the organic phase was dried (MgSO₄), filtered and evaporated. The residue was purified by column chromatography on silica gel (0-100% ethyl acetate in cyclohexane) to provide an orange solid that was further purified by preparative HPLC to afford N-[4-[2-(azetidin-1-yl)phenyl]thiazol-2-yl]-6-methyl-pyridine-3-carboxamide as an off-white solid.

Yield 21 mg (9%). ¹H NMR (400 MHz, DMSO) δ 12.93 (br s, 1H), 9.14 (d, J=2.0 Hz, 1H), 8.35 (dd, J=2.4, 8.2 Hz, 1H), 7.44 (d, J=8.2 Hz, 1H), 7.30 (dd, J=2.0, 7.5 Hz, 1H), 7.23 (dd, J=7.2, 7.5 Hz, 1H), 7.17 (s, 111), 6.80 (dd, J=7.2, 7.2 Hz, 1H), 6.57 (d, J=7.5 Hz, 1H), 3.56 (t, J=7.3 Hz, 4H), 2.57 (s, 3H), 2.15-2.06 (m, 211). m/z: [ESI⁺] 351 (M+H)⁺, (C₁₉H₁₅N₄OS).

### Synthesis of N-[4-(2-chlorophenyl)thiazol-2-yl]-6-methyl-pyridine-3-carboxamide (Compound 318)

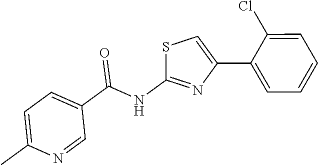 [SMILES: Cc1ccc(C(=O)Nc2nc(-c3ccccc3Cl)cs2)cn1]

To a solution of 4-(2-chlorophenyl)thiazol-2-amine (100 mg, 0.475 mmol) and 6-methylnicotinic acid (130 mg, 0.95 mmol) in anhydrous DCM (2 mL) was added at room temperature triethylamine (0.4 mL, 2.85 mmol) followed by a solution of T3P (50% in ethyl acetate, 0.85 mL, 2.85 mmol). The reaction mixture was heated at 40° C. for 18 hours. After cooling to room temperature, the mixture was partitioned between DCM (10 mL) and water (10 mL). The layers were separated, and the organic phase was washed with water (2×10 mL). The organic layer was dried (MgSO₄), filtered and concentrated under reduced pressure. The residue was purified by preparative HPLC to afford N-[4-(2-chlorophenyl)thiazol-2-yl]-6-methyl-pyridine-3-carboxamide as an off-white solid.

Yield 36 mg (23%). ¹H NMR (400 MHz, DMSO) δ 12.88 (br s, 1H), 9.06 (d, J=2.0 Hz, 1H), 8.27 (dd, J=2.0, 8.0 Hz, 1H), 7.83 (dd, J=2.0, 8.0 Hz, 1H), 7.62 (s, 1H), 7.50 (dd, J=1.3, 7.7 Hz, 1H), 7.41-7.30 (m, 3H), 2.50 (s, 3H). m/z: [ESI⁺] 330 (M+H)⁺, (C₁₆H₁₂CN₃OS).

### Synthesis of N-[4-(2-chlorophenyl)thiazol-2-yl]-2-methyl-pyrimidine-5-carboxamide (Compound 320)

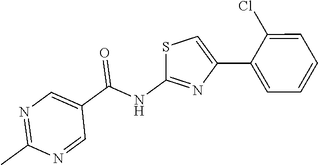 [SMILES: Cc1ncc(C(=O)Nc2nc(-c3ccccc3Cl)cs2)cn1]

Compound N-[4-(2-chlorophenyl)thiazol-2-yl]-2-methyl-pyrimidine-5-carboxamide was prepared from 2-methylpyrimidine-5-carboxylic acid following a similar procedure to that described for the synthesis of N-[4-(2-chlorophenyl)thiazol-2-yl]-6-methyl-pyridine-3-carboxamide, and was isolated as an off-white solid.

Yield 42 mg (27%). ¹H NMR (400 MHz, DMSO) δ 13.0 (br s, 1H), 9.21 (s, 2H), 7.82 (dd, J=1.5, 7.6 Hz, 1H), 7.62 (s, 1H), 7.50 (d, J=7.6 Hz, 1H), 7.41-7.29 (m, 2H), 2.65 (s, 3H). m/z: [ESI⁺] 331 (M+H)⁺, (C₁₅H₁₁ClN₄OS).

### Synthesis of N-[4-(2-chlorophenyl)thiazol-2-yl]-4-(4-methylpiperazin-1-yl)benzamide (Compound 322)

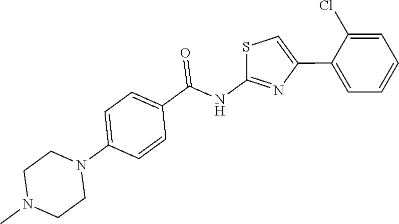 [SMILES: CN1CCN(c2ccc(C(=O)Nc3nc(-c4ccccc4Cl)cs3)cc2)CC1]

Compound N-[4-(2-chlorophenyl)thiazol-2-yl]-4-(4-methylpiperazin-1-yl)benzamide was prepared from 4-(4-methylpiperazin-1-yl)benzoic acid following a similar procedure to that described for the synthesis of N-[4-(2-chlorophenyl)thiazol-2-yl]-6-methyl-pyridine-3-carboxamide, and was isolated as an off-white solid.

Yield 80 mg (40%). ¹H NMR (400 MHz, DMSO) δ 12.43 (br s, 1H), 8.04 (d, J=8.8 Hz, 2H), 7.92 (dd, J=3.2, 7.6 Hz, 1H), 7.62 (s, 1H), 7.57 (dd, J=1.2, 8.0 Hz, 1H), 7.47-7.36 (m, 2H), 7.02 (d, J=8.8 Hz, 211), 3.34 (t, J=4.8 Hz, 4H), 2.45 (t, J=4.8 Hz, 4H), 2.23 (s, 3H).

¹H NMR (400 MHz, CDCl₃) δ 10.03 (br s, 1H), 7.81-7.78 (m, 3H), 7.48 (s, 1H), 7.43 (dd, J=1.2, 8.0 Hz, 11H), 7.31-7.20 (m, 2H), 6.90 (d, J=9.2 Hz, 2H), 3.40 (t, J=5.2 Hz, 4H), 2.60 (t, J=4.8 Hz, 4H), 2.39 (s, 3H). m/z: [ESI⁺] 413 (M+H)⁺, (C₂₁H₂₁ClN₄OS).

### Synthesis of N-[4-(2-chlorophenyl)thiazol-2-yl]-1-methyl-piperidine-4-carboxamide (Compound 324)

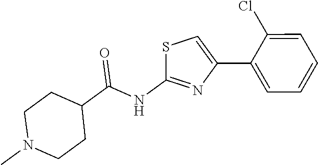 [SMILES: CN1CCC(C(=O)Nc2nc(-c3ccccc3Cl)cs2)CC1]

Compound N-[4-(2-chlorophenyl)thiazol-2-yl]-1-methyl-piperidine-4-carboxamide was prepared from 1-methylpiperidine-4-carboxylic acid following a similar procedure to that described for the synthesis of N-[4-(2-chlorophenyl)thiazol-2-yl]-6-methyl-pyridine-3-carboxamide, and was isolated as an off-white solid.

Yield 42 mg (27%). ¹H NMR (400 MHz, DMSO) δ 12.28 (s, 1H), 7.84 (dd, J=1.5, 7.6 Hz, 1H), 7.60 (s, 1H), 7.56 (dd, J=1.5, 7.6 Hz, 1H), 7.46-7.36 (m, 2H), 2.83 (d, J=11.4 Hz, 2H), 2.50-2.43 (m, 1H), 2.18 (s, 3H), 1.95-1.77 (m, 4H), 1.73-1.61 (m, 2H). m/z: [ESI⁺] 336 (M+H)⁺, (C₁₆H₁₈ClN₃OS).

### Synthesis of N-[4-(2-chlorophenyl)thiazol-2-yl]-4-morpholino-benzamide (Compound 327)

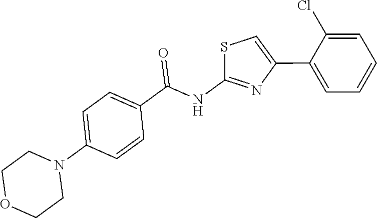 [SMILES: O=C(Nc1nc(-c2ccccc2Cl)cs1)c1ccc(N2CCOCC2)cc1]

Compound N-[4-(2-chlorophenyl)thiazol-2-yl]-4-morpholino-benzamide was prepared from 4-morpholinobenzoic acid following a similar procedure to that described for the synthesis of N-[4-(2-chlorophenyl)thiazol-2-yl]-6-methyl-pyridine-3-carboxamide, and was isolated as an off-white solid.

Yield 47 mg (17%). ¹H NMR (400 MHz, DMSO) δ 12.51 (s, 1H), 8.11 (d, J=9.1 Hz, 2H), 7.96 (dd, J=1.5, 7.6 Hz, 1H), 7.67 (s, 1H), 7.62 (dd, J=1.5, 7.6 Hz, 1H), 7.52-7.41 (m, 2H), 7.09 (d, J=9.1 Hz, 2H), 3.82-3.78 (m, 4H), 3.35-3.33 (m, 4H). m/z: [ESI⁺] 400 (M+H)⁺, (C₂₀H₁₈ClN₃O₂S).

### Synthesis of N-[4-(2-chlorophenyl)thiazol-2-yl]-S-methyl-pyridine-2-carboxamide (Compound 329)

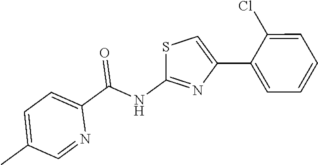 [SMILES: Cc1ccc(C(=O)Nc2nc(-c3ccccc3Cl)cs2)nc1]

Compound N-[4-(2-chlorophenyl)thiazol-2-yl]-5-methyl-pyridine-2-carboxamide was prepared from 5-methylpicolinic acid following a similar procedure to that described for the synthesis of N-[4-(2-chlorophenyl)thiazol-2-yl]-6-methyl-pyridine-3-carboxamide, except that the reaction was re-charged with 5-methylpicolinic acid and coupling reagents and heated for another 18 hours at 45° C. and purified by column chromatography on silica gel (0-20% ethyl acetate in cyclohexane), and was isolated as an off-white solid.

Yield 30 mg (13%). ¹H NMR (400 MHz, DMSO) δ 12.05 (s, 1H), 8.67 (d, J=2.0 Hz, 1H), 8.16 (d, J=8.1 Hz, 1H), 7.98 (dd, J=1.5, 7.7 Hz, 2H), 7.81 (s, 1H), 7.62 (dd, J=1.5, 7.7 Hz, 1H), 7.53-7.42 (m, 2H), 2.50 (s, 3H). m/z: [ESI⁺] 330 (M+H)⁺, (C₁₆H₁₂ClN₃OS).

### Synthesis of N-[4-(2-chlorophenyl)thiazol-2-yl]-5-methoxy-pyrazine-2-carboxamide (Compound 330)

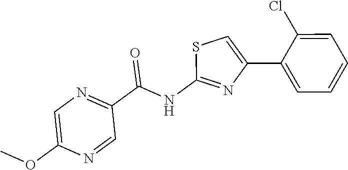 [SMILES: COc1cnc(C(=O)Nc2nc(-c3ccccc3Cl)cs2)cn1]

Compound N-[4-(2-chlorophenyl)thiazol-2-yl]-5-methoxy-pyrazine-2-carboxamide was prepared from 5-methoxypyrazine-2-carboxylic acid following a similar procedure to that described for the synthesis of N-[4-(2-chlorophenyl)thiazol-2-yl]-6-methyl-pyridine-3-carboxamide, except it was purified by sequential trituration with water and diethyl ether, and was isolated as a beige solid.

Yield 73 mg (30%). ¹H NMR (400 MHz, DMSO) δ 12.31 (br s, 1H), 9.02 (s, 1H), 8.51 (s, 1H), 7.97 (dd, J=1.5, 7.6 Hz, 1H), 7.80 (s, 1H), 7.63 (d, J=7.6 Hz, 1H), 7.53-7.42 (m, 2H), 4.10 (s, 3H). m/z: [ESI⁺] 347 (M+H)⁺, (C₁₅H₁₁ClN₄O₂S).

### Synthesis of N-[4-(2-chlorophenyl)thiazol-2-yl]-4-piperazin-1-yl-benzamide (Compound 334)

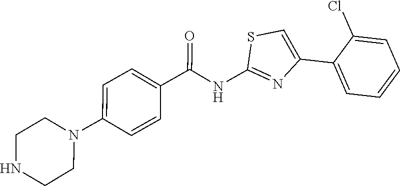 [SMILES: O=C(Nc1nc(-c2ccccc2Cl)cs1)c1ccc(N2CCNCC2)cc1]

tert-Butyl 4-(4-((4-(2-chlorophenyl)thiazol-2-yl)carbamoyl)phenyl)piperazine-1-carboxylate (320 mg, 0.641 mmol) was dissolved at room temperature in a 4 M solution of HCl in 1,4-dioxane (1.6 mL, 6.41 mmol) and the reaction mixture was stirred at room temperature for 18 hours. The reaction mixture was diluted with ethyl acetate (20 mL) and a saturated solution of sodium hydrogen carbonate (20 mL) was added. The precipitate formed was collected by filtration, washed with water (2×10 mL) and dried to afford N-[4-(2-chlorophenyl)thiazol-2-yl]-4-piperazin-1-yl-benzamide as an off-white solid.

Yield 142 mg (56%). ¹H NMR (400 MHz, DMSO) δ 8.09 (d, J=8.8 Hz, 2H), 7.96 (dd, J=1.5, 7.7 Hz, 111), 7.67 (s, 1H), 7.62 (dd, J=1.5, 7.7 Hz, 1H), 7.53-7.41 (m, 2H), 7.06 (d, J=8.8 Hz, 2H), 3.33-3.26 (m, 4H), 2.89-2.83 (m, 4H). Two NH protons obscured. m/z: [ESI⁺] 399 (M+H)⁺, (C₂₀H₁₉ClN₄OS).

### Synthesis of N-[4-(2-chlorophenyl)thiazol-2-yl]-S-morpholino-pyridine-2-carboxamide (Compound 339)

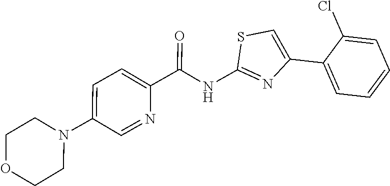 [SMILES: O=C(Nc1nc(-c2ccccc2Cl)cs1)c1ccc(N2CCOCC2)cn1]

To a solution of 4-(2-chlorophenyl)thiazol-2-amine (150 mg, 0.712 mmol) and 5-morpholinopicolinic acid (222 mg, 1.07 mmol) in anhydrous acetonitrile (2 mL) was added at room temperature triethylamine (0.6 mL, 4.27 mmol) followed by a solution of T3P (50% in ethyl acetate, 1.3 mL, 4.27 mmol). The reaction mixture was heated at 50° C. for 18 hours. After cooling to room temperature, the mixture was partitioned between ethyl acetate (20 mL) and water (20 mL). The layers were separated, and the aqueous phase was extracted with ethyl acetate (2×10 mL). The combined organic extracts were dried (MgSO₄), filtered and evaporated. The residue was purified by column chromatography on silica gel (0-25% ethyl acetate in cyclohexane) to afford N-[4-(2-chlorophenyl)thiazol-2-yl]-5-morpholino-pyridine-2-carboxamide as an off-white solid.

Yield 92 mg (32%). ¹H NMR (400 MHz, DMSO) δ 11.65 (s, 1H), 8.44 (d, J=2.8 Hz, 1H), 8.04 (d, J=8.8 Hz, 1H), 7.94 (dd, J=1.5, 7.7 Hz, 1H), 7.72 (s, 1H), 7.58 (dd, J=1.5, 7.7 Hz, 1H), 7.51 (dd, J=2.8, 8.8 Hz, 1H), 7.48-7.38 (m, 2H), 3.79 (dd, J=4.9, 4.9 Hz, 4H), 3.41 (dd, J=4.9, 4.9 Hz, 4H). m/z: [ESI⁺] 401 (M+H)⁺, (C₁₉H₁₇ClN₄O₂S).

### Synthesis of N-[4-(2-chlorophenyl)thiazol-2-yl]-6-morpholino-pyridine-3-carboxamide (Compound 346)

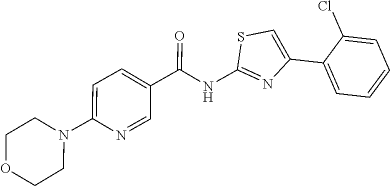 [SMILES: O=C(Nc1nc(-c2ccccc2Cl)cs1)c1ccc(N2CCOCC2)nc1]

Compound N-[4-(2-chlorophenyl)thiazol-2-yl]-6-morpholino-pyridine-3-carboxamide was prepared from 6-morpholinonicotinic acid following a similar procedure to that described for the synthesis of N-[4-(2-chlorophenyl)thiazol-2-yl]-5-morpholino-pyridine-2-carboxamide, except that the reaction was re-charged with 6-morpholinonicotinic acid and coupling reagents and heated for another 18 hours at 50° C., and was isolated as a colourless solid.

Yield 61 mg (21%). ¹H NMR (400 MHz, DMSO) δ 12.59 (s, 1H), 8.91 (d, J=2.1 Hz, 1H), 8.25 (dd, J=2.6, 9.1 Hz, 1H), 7.92 (dd, J=1.5, 7.7 Hz, 1H), 7.65 (s, 1H), 7.58 (dd, J=1.5, 7.7 Hz, 1H), 7.49-7.38 (m, 2H), 6.95 (d, J=9.1 Hz, 1H), 3.73-3.69 (m, 4H), 3.68-3.63 (m, 4H). m/z: [ESI⁺] 401 (M+H)⁺, (C₁₉H₁₇ClN₄O₂S).

### Synthesis of N-[4-(2-chlorophenyl)thiazol-2-yl]-2-methoxy-pyrimidine-5-carboxamide (Compound 345)

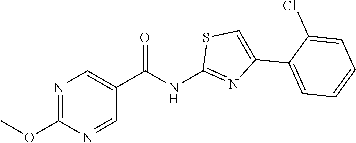 [SMILES: COc1ncc(C(=O)Nc2nc(-c3ccccc3Cl)cs2)cn1]

To a solution of 4-(2-chlorophenyl)thiazol-2-amine (150 mg, 0.712 mmol) and 2-methoxypyrimidine-5-carboxylic acid (165 mg, 1.07 mmol) in anhydrous DCM (4 mL) was added at room temperature triethylamine (0.6 mL, 4.27 mmol) followed by a solution of T3P (50% in ethyl acetate, 1.3 mL, 4.27 mmol). The reaction mixture was heated at 45° C. for 18 hours. After cooling to room temperature, the mixture was partitioned between DCM (20 ml) and water (20 mL). The layers were separated, and the aqueous phase was extracted with DCM (2×10 mL). The combined organic extracts were dried (MgSO₄), filtered and evaporated. The residue was purified by column chromatography on silica gel (0-25% ethyl acetate in cyclohexane) to give an off-white solid that was further purified by preparative HPLC to afford N-[4-(2-chlorophenyl)thiazol-2-yl]-2-methoxy-pyrimidine-5-carboxamide as a colourless solid.

Yield 38 mg (15%). ¹H NMR (400 MHz, DMSO) δ 13.02 (br s, 1H), 9.25 (s, 2H), 7.91 (dd, J=1.5, 7.7 Hz, 1H), 7.69 (s, 1H), 7.59 (dd, J=1.5, 7.7 Hz, 1H), 7.49-7.38 (m, 2H), 4.04 (s, 3H). m/z: [ESI⁺] 347 (M+H)⁺, (C₁₅H₁₁ClN₄O₂S).

### Synthesis of N-[4-(2-chlorophenyl)thiazol-2-yl]-2-morpholino-pyrimidine-5-carboxamide (Compound 358)

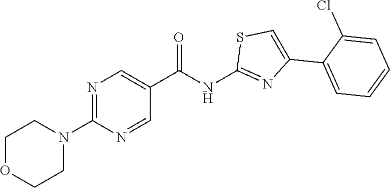 [SMILES: O=C(Nc1nc(-c2ccccc2Cl)cs1)c1cnc(N2CCOCC2)nc1]

To a solution of 4-(2-chlorophenyl)thiazol-2-amine (120 mg, 0.57 mmol) and 2-morpholinopyrimidine-5-carboxylic acid (179 mg, 0.854 mmol) in anhydrous DCM (10 mL) was added at room temperature triethylamine (0.48 mL, 3.42 mmol) followed by a solution of T3P (50% in ethyl acetate, 1 mL, 3.42 mmol). The reaction mixture was heated at 40° C. for 2 days. After cooling to room temperature, the mixture was partitioned between DCM (10 mL) and water (10 mL). The layers were separated, and the organic phase was washed with water (2×10 mL). The organic layer was dried (MgSO₄), filtered and concentrated under reduced pressure. The residue was purified by column chromatography on silica gel (0-25% ethyl acetate in cyclohexane) to afford N-[4-(2-chlorophenyl)thiazol-2-yl]-2-morpholino-pyrimidine-5-carboxamide as a colourless solid.

Yield 150 mg (65%). ¹H NMR (400 MHz, DMSO) δ 12.72 (br s, 1H), 9.05 (s, 2H), 7.91 (dd, J=1.5, 7.7 Hz, 1H), 7.67 (s, 1H), 7.58 (dd, J=1.5, 7.7 Hz, 1H), 7.49-7.38 (m, 2H), 3.89-3.85 (m, 4H), 3.72-3.68 (m, 4H). m/z: [ESI⁺] 402 (M+H)⁺, (C₁₅H₁₆ClN₅O₂S).

### Synthesis of 2-methoxy-N-[4-(4-methyl-3-pyridyl)thiazol-2-yl]pyrimidine-5-carboxamide (Compound 349)

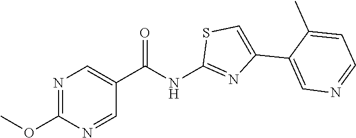 [SMILES: COc1ncc(C(=O)Nc2nc(-c3cnccc3C)cs2)cn1]

To a degassed mixture of N-(4-bromothiazol-2-yl)-2-methoxypyrimidine-5-carboxamide (122 mg, 0.388 mmol) and 4-methyl-3-(4,4,5,5-tetramethyl-1,3,2-dioxaborolan-2-yl)pyridine (170 mg, 0.766 mmol) in 1,4-dioxane (4 mL) was added at room temperature bis(di-tert-butyl(4-dimethylamino-phenyl)phosphine)dichloropalladium(II) (55 mg, 0.078 mmol) and a solution of cesium carbonate (758 mg, 2.33 mmol) in water (0.5 mL). The reaction mixture was heated at 120° C. in a microwave for 1 hour. The reaction was re-charged with 4-methyl-3-(4,4,5,5-tetramethyl-1,3,2-dioxaborolan-2-yl)pyridine (170 mg, 0.766 mmol) and bis(di-tert-butyl(4-dimethylaminophenyl)phosphine)dichloropalladium(II) (55 mg, 0.078 mmol) and heated at 120° C. in a microwave for 1 hour. After cooling to room temperature, the reaction mixture was diluted with ethyl acetate (20 mL) and filtered through Celite. The filtrate was washed with brine (10 mL), dried (MgSO₄), filtered and evaporated. The residue was purified by column chromatography on silica gel (0-100% ethyl acetate in cyclohexane) to give a yellow solid that was further purified by preparative HPLC to afford 2-methoxy-N-[4-(4-methyl-3-pyridyl)thiazol-2-yl]pyrimidine-5-carboxamide as an off-white solid.

Yield 9 mg (4%). ¹H NMR (400 MHz, DMSO) δ 12.99 (br s, 1H), 9.25 (s, 2H), 8.79 (s, 1H), 8.43 (d, J=5.0 Hz, 1H), 7.54 (s, 1H), 7.35 (d, J=5.0 Hz, 1H), 4.04 (s, 3H), 2.51 (s, 3H). m/z: [ESI⁺] 328 (M+H)⁺, (C₁₅H₁₃N₅O₂S).

### Synthesis of 2-methoxy-N-[4-(2-methyl-3-pyridyl)thiazol-2-yl]pyrimidine-5-carboxamide (Compound 355)

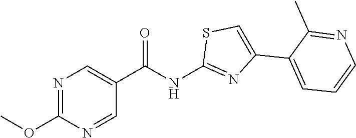 [SMILES: COc1ncc(C(=O)Nc2nc(-c3cccnc3C)cs2)cn1]

To a degassed mixture of N-(4-bromothiazol-2-yl)-2-methoxypyrimidine-5-carboxamide (150 mg, 0.476 mmol) and (2-methylpyridin-3-yl)boronic acid (130 mg, 0.952 mmol) in 1,4-dioxane (4 mL) was added at room temperature bis(di-tert-butyl(4-dimethylaminophenyl)phosphine)dichloropalladium(II) (34 mg, 0.048 mmol) and a solution of cesium carbonate (465 mg, 1.43 mmol) in water (0.5 mL). The reaction mixture was heated at 120° C. in a microwave for 1 hour. After cooling to room temperature, the reaction mixture was diluted with ethyl acetate (20 mL) and filtered through Celite. The filtrate was evaporated and the residue purified by column chromatography on silica gel (0-50% ethyl acetate in cyclohexane) to afford 2-methoxy-N-[4-(2-methyl-3-pyridyl)thiazol-2-yl]pyrimidine-5-carboxamide as an off-white solid.

Yield 67 mg (43%). ¹H NMR (400 MHz, DMSO) δ 13.03 (br s, 1H), 9.29 (s, 2H), 8.51 (dd, J=1.8, 4.7 Hz, 1H), 8.04 (dd, J=1.8, 7.8 Hz, 1H), 7.57 (s, 1H), 7.38 (dd, J=4.7, 7.8 Hz, 1H), 4.08 (s, 3H), 2.72 (s, 3H). m/z: [ESI⁺] 328 (M+H)⁺, (C₁₅H₁₃N₅O₂S).

### Synthesis of 2-methoxy-N-[4-(2-methoxy-3-pyridyl)thiazol-2-yl]pyrimidine-5-carboxamide (Compound 356)

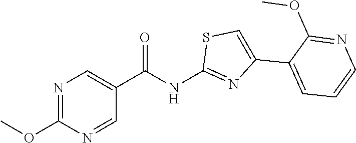 [SMILES: COc1ncc(C(=O)Nc2nc(-c3cccnc3OC)cs2)cn1]

Compound 2-methoxy-N-[4-(2-methoxy-3-pyridyl)thiazol-2-yl]pyrimidine-5-carboxamide was prepared from 2-methoxy-3-(4,4,5,5-tetramethyl-1,3,2-dioxaborolan-2-yl)pyridine following a similar procedure to that described for the synthesis of 2-methoxy-N-[4-(2-methyl-3-pyridyl)thiazol-2-yl]pyrimidine-5-carboxamide, and was isolated as a colourless solid.

Yield 95 mg (58%). ¹H NMR (400 MHz, DMSO) δ 12.93 (br s, 1H), 9.25 (s, 2H), 8.46 (dd, J=1.9, 7.5 Hz, 1H), 8.19 (dd, J=1.9, 4.9 Hz, 1H), 7.86 (s, 1H), 7.16 (dd, J=4.9, 7.5 Hz, 1H), 4.05 (s, 3H), 4.04 (s, 3H). m/z: [ESI⁺] 344 (M+H)⁺, (C₁₅H₁₃N₅O₃S).

### Synthesis of N-[4-(2-methoxyphenyl)thiazol-2-yl]-4-morpholino-benzamide (Compound 351)

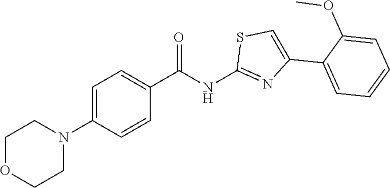 [SMILES: COc1ccccc1-c1csc(NC(=O)c2ccc(N3CCOCC3)cc2)n1]

To a degassed mixture of N-(4-bromothiazol-2-yl)-4-morpholinobenzamide (120 mg, 0.326 mmol) and (2-methoxyphenyl)boronic acid (99 mg, 0.652 mmol) in 1,4-dioxane (4 mL) was added at room temperature bis(di-tert-butyl(4-dimethylaminophenyl)phosphine)dichloropalladium(II) (23 mg, 0.033 mmol) and a solution of cesium carbonate (319 mg, 0.978 mmol) in water (0.5 mL). The reaction mixture was heated at 120° C. in a microwave for 1 hour. The reaction was re-charged with (2-methoxyphenyl)boronic acid (99 mg, 0.652 mmol) and heated at 120° C. in a microwave for 1 hour. After cooling to room temperature, the reaction mixture was diluted with ethyl acetate (20 mL) and filtered through Celite. The filtrate was evaporated and the residue purified by preparative HPLC to afford N-[4-(2-methoxyphenyl)thiazol-2-yl]-4-morpholino-benzamide as an off-white solid.

Yield 20 mg (16%). ¹H NMR (400 MHz, DMSO) δ 12.35 (br s, 1H), 8.17 (dd, J=1.8, 7.8 Hz, 1H), 8.07 (d, J=9.0 Hz, 2H), 7.67 (s, 1H), 7.33 (dd, J=6.8, 9.0 Hz, 1H), 7.15 (d, J=7.8 Hz, 1H), 7.07-7.03 (m, 3H), 3.94 (s, 3H), 3.79-3.74 (m, 4H), 3.33-3.29 (m, 4H). m/z: [ESI⁺] 396 (M+H)⁺, (C₂₁H₂₁N₃O₃S).

### Synthesis of 4-morpholino-N-[4-(3-pyridyl)thiazol-2-yl]benzamide (Compound 352)

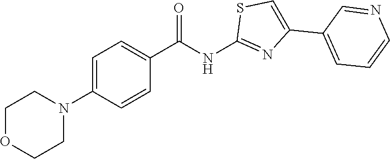 [SMILES: O=C(Nc1nc(-c2cccnc2)cs1)c1ccc(N2CCOCC2)cc1]

Compound 4-morpholino-N-[4-(3-pyridyl)thiazol-2-yl]benzamide was prepared from pyridin-3-ylboronic acid following a similar procedure to that described for the synthesis of N-[4-(2-methoxyphenyl)thiazol-2-yl]-4-morpholino-benzamide, and was isolated as a yellow solid.

Yield 31 mg (26%). ¹H NMR (400 MHz, DMSO) δ 12.55 (br s, 1H), 9.22 (d, J=1.5 Hz, 1H), 8.58 (dd, J=1.5, 4.8 Hz, 1H), 8.35-8.31 (m, 1H), 8.12 (d, J=9.1 Hz, 2H), 7.86 (s, 1H), 7.53 (dd, J=4.8, 7.3 Hz, 1H), 7.09 (d, J=9.1 Hz, 2H), 3.83-3.77 (m, 4H), 3.36-3.33 (m, 4H). m/z: [ESI⁺] 367 (M+H)⁺, (C₁₉H₁₅N₄O₂S).

### Synthesis of N-[4-(3,5-dimethoxyphenyl)thiazol-2-yl]-4-morpholino-benzamide (Compound 353)

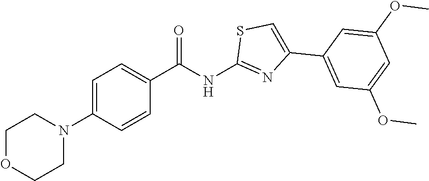 [SMILES: COc1cc(OC)cc(-c2csc(NC(=O)c3ccc(N4CCOCC4)cc3)n2)c1]

Compound N-[4-(3,5-dimethoxyphenyl)thiazol-2-yl]-4-morpholino-benzamide was prepared from 2-(3,5-dimethoxyphenyl)-4,4,5,5-tetramethyl-1,3,2-dioxaborolane following a similar procedure to that described for the synthesis of N-[4-(2-methoxyphenyl)thiazol-2-yl]-4-morpholino-benzamide, and was isolated as an off-white solid.

Yield 40 mg (29%). ¹H NMR (400 MHz, DMSO) δ 12.40 (br s, 1H), 8.07 (d, J=9.0 Hz, 2H), 7.70 (s, 111), 7.15 (d, J=2.3 Hz, 2H), 7.05 (d, J=9.0 Hz, 2H), 6.48 (t, J=2.3 Hz, 1H), 3.81 (s, 6H), 3.78-3.74 (m, 4H), 3.33-3.29 (m, 4H). m/z: [ESI⁺] 426 (M+H)⁺, (C₂₂H₂₃N₃O₄S).

### Synthesis of N-[4-(1-methylpyrazol-4-yl)thiazol-2-yl]-4-morpholino-benzamide (Compound 354)

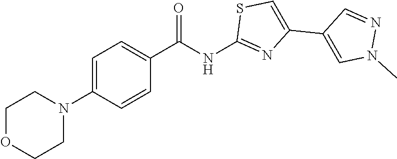 [SMILES: Cn1cc(-c2csc(NC(=O)c3ccc(N4CCOCC4)cc3)n2)cn1]

Compound N-[4-(1-methylpyrazol-4-yl)thiazol-2-yl]-4-morpholino-benzamide was prepared from 1-methyl-4-(4,4,5,5-tetramethyl-1,3,2-dioxaborolan-2-yl)-1H-pyrazole following a similar procedure to that described for the synthesis of N-[4-(2-methoxyphenyl)thiazol-2-yl]-4-morpholino-benzamide, except that the reaction was not re-charged, and was isolated as an off-white solid.

Yield 18 mg (15%). ¹H NMR (400 MHz, DMSO) δ 12.39 (br s, 1H), 8.05 (d, J=9.2 Hz, 2H), 8.00 (s, 111), 7.81 (s, 1H), 7.19 (s, 1H), 7.04 (d, J=9.2 Hz, 2H), 3.89 (s, 311), 3.78-3.74 (m, 411), 3.33-3.29 (m, 411). m/z: [ESI⁺] 370 (M+H), (C₁₅H₁₉N₅O₂S).

### Synthesis of N-[4-(3-methoxyphenyl)thiazol-2-yl]-4-morpholino-benzamide (Compound 357)

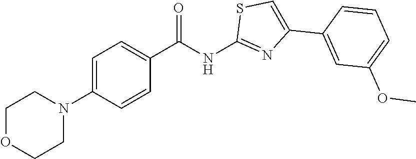 [SMILES: COc1cccc(-c2csc(NC(=O)c3ccc(N4CCOCC4)cc3)n2)c1]

Compound N-[4-(3-methoxyphenyl)thiazol-2-yl]-4-morpholino-benzamide was prepared from (3-methoxyphenyl)boronic acid following a similar procedure to that described for the synthesis of N-[4-(2-methoxyphenyl)thiazol-2-yl]-4-morpholino-benzamide, except that the reaction was not re-charged, and was isolated as an off-white solid.

Yield 40 mg (31%). ¹H NMR (400 MHz, DMSO) δ 12.42 (s, 1H), 8.07 (d, J=9.2 Hz, 2H), 7.68 (s, 1H), 7.56-7.53 (m, 2H), 7.36 (dd, J=8.0, 8.0 Hz, 1H), 7.05 (d, J=9.2 Hz, 2H), 6.93-6.90 (m, 1H), 3.83 (s, 311), 3.78-3.74 (m, 4H), 3.33-3.29 (m, 4H). m/z: [ESI⁺] 396 (M+H)⁺, (C₂₁H₂₁N₃O₃S).

### Synthesis of 4-morpholino-N-(4-phenylthiazol-2-yl)benzamide (Compound 359)

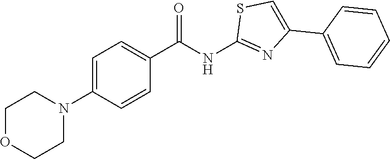 [SMILES: O=C(Nc1nc(-c2ccccc2)cs1)c1ccc(N2CCOCC2)cc1]

To a degassed mixture of N-(4-bromothiazol-2-yl)-4-morpholinobenzamide (120 mg, 0.326 mmol) and phenylboronic acid (79 mg, 0.652 mmol) in 1,4-dioxane (4 mL) was added at room temperature bis(di-tert-butyl(4-dimethylaminophenyl)phosphine)dichloropalladium(II) (23 mg, 0.033 mmol) and a solution of cesium carbonate (319 mg, 0.978 mmol) in water (1 mL). The reaction mixture was heated at 120° C. in a microwave for 1 hour. After cooling to room temperature, the reaction mixture was diluted with ethyl acetate (20 mL) and filtered through Celite. The filtrate was evaporated and the residue purified by column chromatography on silica gel (0-25% ethyl acetate in cyclohexane) to afford, after trituration with diethyl ether and petrol ether, 4-morpholino-N-(4-phenylthiazol-2-yl)benzamide as an off-white solid.

Yield 50 mg (42%). ¹H NMR (400 MHz, DMSO) δ 12.44 (s, 1H), 8.08 (d, J=9.0 Hz, 2H), 7.97 (d, J=7.3 Hz, 2H), 7.65 (s, 1H), 7.46 (dd, J=7.6, 7.6 Hz, 2H), 7.35 (dd, J=7.3, 7.3 Hz, 1H), 7.05 (d, J=9.0 Hz, 211), 3.78-3.74 (m, 4H), 3.33-3.29 (m, 4H). m/z: [ESI⁺] 366 (M+H)⁺, (C₂₀H₁₉N₃O₂S).

### Synthesis of N-[4-(2-cyanophenyl)thiazol-2-yl]-4-morpholino-benzamide (Compound 361)

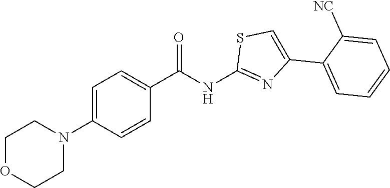 [SMILES: N#Cc1ccccc1-c1csc(NC(=O)c2ccc(N3CCOCC3)cc2)n1]

To a degassed mixture of N-(4-bromothiazol-2-yl)-4-morpholinobenzamide (120 mg, 0.326 mmol) and (2-cyanophenyl)boronic acid (96 mg, 0.652 mmol) in 1,4-dioxane (4 ml) was added at room temperature bis(di-tert-butyl(4-dimethylaminophenyl)phosphine)dichloropalladium(II) (23 mg, 0.033 mmol) and a solution of cesium carbonate (319 mg, 0.978 mmol) in water (1 mL). The reaction mixture was heated at 120° C. in a microwave for 1 hour. After cooling to room temperature, the reaction mixture was diluted with ethyl acetate (20 ml) and filtered through Celite. The filtrate was evaporated and the residue purified by column chromatography on silica gel (0-50% ethyl acetate in cyclohexane) to afford an orange solid. Further purification by preparative SFC chromatography afforded N-[4-(2-cyanophenyl)thiazol-2-yl]-4-morpholino-benzamide as a white solid.

Yield 26 mg (20%). ¹H NMR (400 MHz, DMSO) δ 12.59 (s, 1H), 8.12 (d, J=9.1 Hz, 2H), 8.08 (d, J=7.5 Hz, 1H), 7.99 (dd, J=0.9, 7.7 Hz, 1H), 7.85 (dd, J=1.5, 7.7 Hz, 1H), 7.83 (s, 1H), 7.61 (dd, J=1.5, 7.5 Hz, 1H), 7.10 (d, J=9.1 Hz, 2H), 3.82-3.79 (m, 4H), 3.37-3.34 (m, 4H). m/z: [ESI⁺] 391 (M+H)⁺, (C₂₁H₁₅N₄O₂S).

### Synthesis of N-[4-(2-chlorophenyl)thiazol-2-yl]-4-morpholino-piperidine-1-carboxamide (Compound 360)

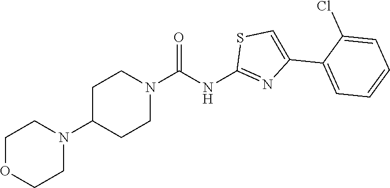 [SMILES: O=C(Nc1nc(-c2ccccc2Cl)cs1)N1CCC(N2CCOCC2)CC1]

To a solution of phenyl (4-(2-chlorophenyl)thiazol-2-yl)carbamate (236 mg, 0.713 mmol) in pyridine (3 mL) was added at room temperature triethylamine (0.3 mL, 2.14 mmol) and 4-(piperidin-4-yl)morpholine (0.15 mL, 1.07 mmol). The reaction was stirred at room temperature for 4 hours. The reaction was partitioned between ethyl acetate (20 mL) and water (20 mL) and the layers were separated. The aqueous layer was extracted with ethyl acetate (2×20 mL). The organic layers were combined, dried (MgSO₄) and filtered. The filtrate was evaporated and the residue purified by preparative HPLC to afford N-[4-(2-chlorophenyl)thiazol-2-yl]-4-morpholino-piperidine-1-carboxamide as a white solid.

Yield 93 mg (32%). ¹H NMR (400 MHz, DMSO) δ 11.05 (br s, 1H), 7.93 (dd, J=1.8, 7.8 Hz, 1H), 7.58 (dd, J=1.8, 7.8 Hz, 1H), 7.52-7.38 (m, 3H), 4.27 (d, J=12.9 Hz, 2H), 3.64-3.59 (m, 4H), 2.90 (t, J=12.9 Hz, 2H), 2.54-2.49 (m, 4H), 2.47-2.39 (m, 1H), 1.86 (d, J=10.9 Hz, 2H), 1.43-1.31 (m, 2H). m/z: [ESI⁺]407 (M+H)⁺, (C₁₉H₁₃ClN₄O₂S).

### Synthesis of 4-morpholino-N-[4-(2-pyridyl)thiazol-2-yl]benzamide (Compound 370)

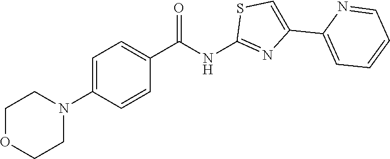 [SMILES: O=C(Nc1nc(-c2ccccn2)cs1)c1ccc(N2CCOCC2)cc1]

Compound 4-morpholino-N-[4-(2-pyridyl)thiazol-2-yl]benzamide was prepared from 4-(pyridin-2-yl)thiazol-2-amine following a similar procedure to that described for the synthesis of N-[4-(2-chlorophenyl)thiazol-2-yl]-2-morpholino-pyrimidine-5-carboxamide and was isolated as an off-white solid.

Yield 110 mg (31%). ¹H NMR (400 MHz, DMSO) δ 12.49 (br s, 1H), 8.65-8.61 (m, 1H), 8.08 (d, J=9.2 Hz, 2H), 8.04 (d, J=7.8 Hz, 1H), 7.91 (dd, J=7.5, 7.8 Hz, 1H), 7.86 (s, 1H), 7.36 (dd, J=4.8, 7.5 Hz, 1H), 7.06 (d, J=9.2 Hz, 2H), 3.78-3.74 (m, 4H), 3.33-3.29 (m, 4H). m/z: [ESI⁺] 367 (M+H)⁺, (C₁₉H₁₈N₄O₂S).

### Synthesis of N-[4-(2-methyl-3-pyridyl)thiazol-2-yl]-4-morpholino-benzamide (Compound 369)

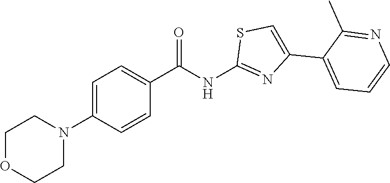 [SMILES: Cc1ncccc1-c1csc(NC(=O)c2ccc(N3CCOCC3)cc2)n1]

To a degassed solution of N-(4-bromothiazol-2-yl)-4-morpholinobenzamide (120 mg, 0.326 mmol) in 1,4-dioxane (4 mL) and water (1 mL) was added at room temperature (2-methylpyridin-3-yl) boronic acid (89 mg, 0.652 mmol), bis(di-tert-butyl(4-dimethylaminophenyl)phosphine)dichloropalladium(II) (23 mg, 0.033 mmol) and cesium carbonate (319 mg, 0.978 mmol). The reaction was heated in a microwave at 120° C. for 1 hour and cooled to room temperature. The reaction mixture was diluted with ethyl acetate (20 mL) and then filtered through Celite. The filtrate and combined washings were collected. The solvent was evaporated to give an orange oil, which was purified by column chromatography on silica gel (0-75% ethyl acetate in cyclohexane) to afford a yellow solid. Further purification by preparative SFC chromatography afforded N-[4-(2-methyl-3-pyridyl)thiazol-2-yl]-4-morpholino-benzamide as a white solid.

Yield 38 mg (31%). ¹H NMR (400 MHz, DMSO) δ 12.45 (br s, 1H), 8.46 (dd, J=1.8, 4.8 Hz, 1H), 8.07 (d, J=9.2 Hz, 2H), 8.01 (dd, J=1.8, 7.8 Hz, 1H), 7.43 (s, 1H), 7.33 (dd, J=4.8, 7.8 Hz, 1H), 7.05 (d, J=9.2 Hz, 2H), 3.78-3.74 (m, 4H), 3.31-3.29 (m, 4H), 2.68 (s, 3H). m/z: [ESI⁺] 381 (M+H)⁺, (C₂₀H₁₀N₄O₂S).

### Synthesis of N-[4-(2-chlorophenyl)oxazol-2-yl]-4-morpholino-benzamide (Compound 368)

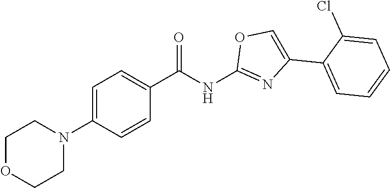 [SMILES: O=C(Nc1nc(-c2ccccc2Cl)co1)c1ccc(N2CCOCC2)cc1]

Compound N-[4-(2-chlorophenyl)oxazol-2-yl]-4-morpholino-benzamide was prepared from 4-(2-chlorophenyl)oxazol-2-amine following a similar procedure to that described for the synthesis of N-[4-(2-chlorophenyl)thiazol-2-yl]-2-morpholino-pyrimidine-5-carboxamide and was isolated as an off-white solid.

Yield 33 mg (56%). ¹H NMR (400 MHz, DMSO) δ 11.41 (s, 1H), 8.55 (s, 1H), 8.06 (dd, J=1.8, 7.8 Hz, 1H), 7.94 (d, J=9.0 Hz, 2H), 7.59 (dd, J=1.1, 7.8 Hz, 1H), 7.49 (dd, J=7.6, 7.6 Hz, 1H), 7.40 (dd, J=7.6, 7.6 Hz, 1H), 7.05 (d, J=9.0 Hz, 2H), 3.78-3.74 (m, 4H), 3.33-3.29 (m, 4H). m/z: [ESI⁺] 384 (M+H)⁺, (C₂₀H₁₈ClN₃O₃).

### Synthesis of N-[4-(2-chlorophenyl)thiazol-2-yl]-5-morpholino-pyrazine-2-carboxamide (Compound 367)

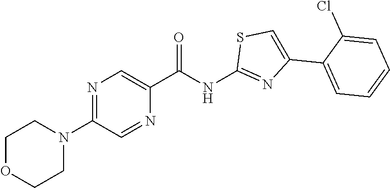 [SMILES: O=C(Nc1nc(-c2ccccc2Cl)cs1)c1cnc(N2CCOCC2)cn1]

To a suspension of 5-chloropyrazine-2-carboxylic acid hydrochloride (58 mg, 0.237 mmol) and 4-(2-chlorophenyl)thiazol-2-amine (50 mg, 0.237 mmol) in acetonitrile (0.5 mL) was added 1-methylimidazole (68 mg, 0.831 mmol) and N,N,N′,N′-tetramethylchloroformamidinium hexafluorophosphate (TCFH) (80 mg, 0.285 mmol) at room temperature. The reaction was stirred at room temperature for 2 hours, after which time an off-white solid had formed. The reaction was diluted with acetonitrile (2 mL) and the solid was collected by filtration. The solid was washed with acetonitrile/water (2:1, 10 mL) and air dried to afford N-[4-(2-chlorophenyl)thiazol-2-yl]-5-morpholino-pyrazine-2-carboxamide as a white solid.

Yield 37 mg (39%). ¹H NMR (400 MHz, DMSO) δ 11.83 (br s, 1H), 8.82 (d, J=1.3 Hz, 111), 8.41 (d, J=1.3 Hz, 111), 7.93 (dd, J=1.5, 7.8 Hz, 1H), 7.72 (s, 111), 7.58 (dd, J=1.5, 7.8 Hz, 1H), 7.48-7.38 (m, 2H), 3.80-3.73 (m, 8H). m/z: [ESI⁺] 402 (M+H)⁺, (C₁₈H₁₆ClN₅O₂S).

### Synthesis of N-[4-(2-chlorophenyl)thiazol-2-yl]-6-morpholino-pyridazine-3-carboxamide (Compound 366)

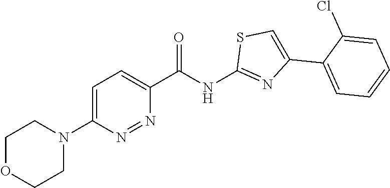 [SMILES: O=C(Nc1nc(-c2ccccc2Cl)cs1)c1ccc(N2CCOCC2)nn1]

Compound N-[4-(2-chlorophenyl)thiazol-2-yl]-6-morpholino-pyridazine-3-carboxamide was prepared from 4-(2-chlorophenyl)oxazol-2-amine and 6-morpholinopyridazine-3-carboxylic acid hydrochloride following a similar procedure to that described for the synthesis of N-[4-(2-chlorophenyl)thiazol-2-yl]-5-morpholino-pyrazine-2-carboxamide and was isolated as a white solid.

Yield 94 mg (49%). ¹H NMR (400 MHz, DMSO) δ 12.31 (br s, 1H), 8.03 (d, J=9.7 Hz, 1H), 7.94 (dd, J=1.5, 7.8 Hz, 1H), 7.75 (s, 1H), 7.58 (dd, J=1.5, 7.8 Hz, 1H), 7.49-7.38 (m, 3H), 3.78 (s, 8H). m/z: [ESI⁺]402 (M+H)⁺, (C₁₅H₁₆ClN₅O₂S).

### Synthesis of N-[4-(2-chlorophenyl)thiazol-2-yl]-4-(4-methylsulfonylpiperazin-1-yl)benzamide (Compound 365)

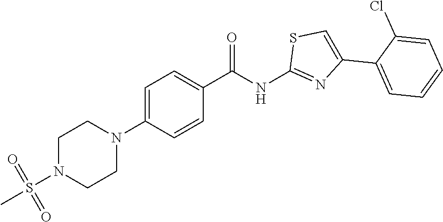 [SMILES: CS(=O)(=O)N1CCN(c2ccc(C(=O)Nc3nc(-c4ccccc4Cl)cs3)cc2)CC1]

To a solution of N-[4-(2-chlorophenyl)thiazol-2-yl]-4-piperazin-1-yl-benzamide (96 mg, 0.241 mmol) in DCM (3 ml) at 0° C. was added triethylamine (84 μL, 0.602 mmol) followed by methanesulfonyl chloride (22 μL, 0.289 mmol). The reaction was allowed to warm to room temperature and stirred for 1 hour. The reaction mixture was partitioned between DCM (10 ml) and water (10 ml) and the layers were separated. The aqueous layer was further extracted with DCM (2×10 mL) and the organic layers combined. The combined organic extracts were dried by passing through a Phase Separator cartridge. The DCM was removed by evaporation to give a residue. Addition of DCM (10 mL) yielded a precipitate, which was collected by filtration to afford a brown solid. The brown solid was purified by column chromatography on silica gel (0-75% ethyl acetate in cyclohexane) to afford a white solid. Further purification by preparative SFC chromatography afforded N-[4-(2-chlorophenyl)thiazol-2-yl]-4-(4-methylsulfonylpiperazin-1-yl)benzamide as an off-white solid.

Yield 5 mg (4%). ¹H NMR (400 MHz, DMSO) δ 12.49 (br s, 1H), 8.08 (d, J=9.2 Hz, 2H), 7.92 (dd, J=1.5, 7.7 Hz, 1H), 7.64 (s, 1H), 7.58 (dd, J=1.5, 7.7 Hz, 1H), 7.46 (dd, J=7.5, 7.5 Hz, 1H), 7.40 (dd, J=7.5, 7.5 Hz, 1H), 7.09 (d, J=9.2 Hz, 2H), 3.51-3.47 (m, 4H), 3.28-3.24 (m, 4H), 2.94 (s, 3H). m/z: [ESI⁺] 477 (M+H)⁺, (C₂₁H₂₁ClN₄O₃S₂).

### Synthesis of N-[4-(2-chlorophenyl)thiazol-2-yl]-1-tetrahydropyran-4-yl-azetidine-3-carboxamide (Compound 363)

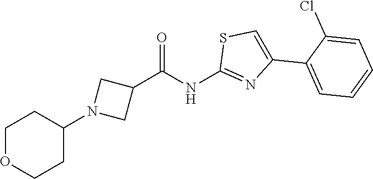 [SMILES: O=C(Nc1nc(-c2ccccc2Cl)cs1)C1CN(C2CCOCC2)C1]

Compound N-[4-(2-chlorophenyl)thiazol-2-yl]-1-tetrahydropyran-4-yl-azetidine-3-carboxamide was prepared from 4-(2-chlorophenyl)thiazol-2-amine and 1-(tetrahydro-2H-pyran-4-yl)azetidine-3-carboxylic acid following a similar procedure to that described for the synthesis of N-[4-(2-chlorophenyl)thiazol-2-yl]-5-morpholino-pyrazine-2-carboxamide except after completion of the reaction the mixture was diluted with DCM and washed with water. The layers were separated using a phase separator and the solvent evaporated to give a residue, which was purified by column chromatography on silica gel (0-10% methanol in DCM) to afford a white solid. The white solid was further purified by a trituration from ethyl acetate to afford N-[4-(2-chlorophenyl)thiazol-2-yl]-1-tetrahydropyran-4-yl-azetidine-3-carboxamide as a white solid.

Yield 16 mg (9%). ¹H NMR (400 MHz, DMSO) δ 12.32 (br s, 1H), 7.84 (dd, J=1.5, 7.6 Hz, 1H), 7.62 (s, 1H), 7.56 (dd, J=1.5, 7.8 Hz, 1H), 7.46-7.36 (m, 2H), 3.84-3.77 (m, 2H), 3.49-3.39 (m, 3H), 3.34-3.25 (m, 2H), 3.23-3.18 (m, 2H), 2.27-2.19 (m, 1H), 1.62-1.58 (m, 2H), 1.18-1.07 (m, 2H). m/z: [ESI⁺] 378 (M+H)⁺, (C₁₈H₂₀ClN₃O₂S).

### Synthesis of N-[4-(2-methyl-3-pyridyl)thiazol-2-yl]-5-morpholino-pyridine-2-carboxamide (Compound 371)

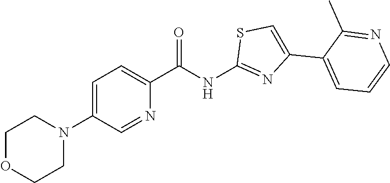 [SMILES: Cc1ncccc1-c1csc(NC(=O)c2ccc(N3CCOCC3)cn2)n1]

Compound N-[4-(2-methyl-3-pyridyl)thiazol-2-yl]-5-morpholino-pyridine-2-carboxamide was prepared from 4-(2-methylpyridin-3-yl)thiazol-2-amine and 5-morpholinopicolinic acid following a similar procedure to that described for the synthesis of N-[4-(2-chlorophenyl)thiazol-2-yl]-2-morpholino-pyrimidine-5-carboxamide and was isolated as an off-white solid.

Yield 31 mg (19%). ¹H NMR (400 MHz, DMSO) δ 11.59 (s, 1H), 8.40-8.35 (m, 2H), 7.98-7.92 (m, 2H), 7.46-7.42 (m, 2H), 7.25 (dd, J=4.8, 7.6 Hz, 1H), 3.74-3.67 (m, 4H), 3.37-3.30 (m, 4H), 2.61 (s, 3H). m/z: [ESI⁺] 382 (M+H)⁺, (C₁₉H₁₉N₅O₂S).

### Synthesis of N-[4-(2-methyl-3-pyridyl)thiazol-2-yl]-5-morpholino-pyrazine-2-carboxamide (Compound 372)

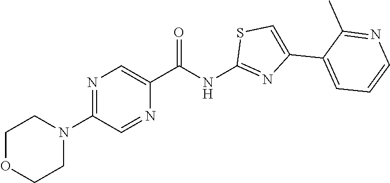 [SMILES: Cc1ncccc1-c1csc(NC(=O)c2cnc(N3CCOCC3)cn2)n1]

Compound N-[4-(2-methyl-3-pyridyl)thiazol-2-yl]-5-morpholino-pyrazine-2-carboxamide was prepared from 4-(2-methylpyridin-3-yl)thiazol-2-amine and 5-chloropyrazine-2-carboxylic acid hydrochloride following a similar procedure to that described for the synthesis of N-[4-(2-chlorophenyl)thiazol-2-yl]-5-morpholino-pyrazine-2-carboxamide except the compound was purified by column chromatography on silica gel (0-30% ethanol in ethyl acetate (1:3) in cyclohexane) to afford an off-white solid, which was triturated from ethyl acetate to afford N-[4-(2-methyl-3-pyridyl)thiazol-2-yl]-5-morpholino-pyrazine-2-carboxamide as an off-white solid.

Yield 28 mg (17%). ¹H NMR (400 MHz, DMSO) δ 11.87 (s, 1H), 8.86 (d, J=1.5 Hz, 1H), 8.50 (dd, J=1.8, 4.8 Hz, 1H), 8.45 (d, J=1.5 Hz, 1H), 8.05 (dd, J=1.8, 7.8 Hz, 1H), 7.55 (s, 1H), 7.37 (dd, J=4.8, 7.8 Hz, 111), 3.84-3.76 (m, 8H), 2.72 (s, 3H). m/z: [ESI⁺] 383 (M+H)⁺, (C₁₈H₁₈N₆O₂S).

### Synthesis of N-[4-(2-methyl-3-pyridyl)thiazol-2-yl]-6-morpholino-pyridazine-3-carboxamide (Compound 373)

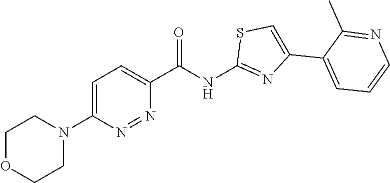 [SMILES: Cc1ncccc1-c1csc(NC(=O)c2ccc(N3CCOCC3)nn2)n1]

Compound N-[4-(2-methyl-3-pyridyl)thiazol-2-yl]-6-morpholino-pyridazine-3-carboxamide was prepared from 4-(2-methylpyridin-3-yl)thiazol-2-amine and 6-morpholinopyridazine-3-carboxylic acid hydrochloride following a similar procedure to that described for the synthesis of N-[4-(2-chlorophenyl)thiazol-2-yl]-5-morpholino-pyrazine-2-carboxamide and was isolated as an off-white solid.

Yield 60 mg (37%). ¹H NMR (400 MHz, DMSO) δ 12.38 (s, 1H), 8.51 (dd, J=1.8, 4.8 Hz, 1H), 8.07 (d, J=9.6 Hz, 1H), 8.06 (dd, J=1.8, 7.7 Hz, 1H), 7.58 (s, 1H), 7.49 (d, J=9.6 Hz, 1H), 7.38 (dd, J=4.8, 7.7 Hz, 1H), 3.82 (s, 8H), 2.73 (s, 3H). m/z: [ESI⁺] 383 (M+H)⁺, (C₁₈H₁₈N₆O₂S).

### Synthesis of N-[4-(2-methyl-3-pyridyl)thiazol-2-yl]-6-morpholino-pyridine-3-carboxamide (Compound 375)

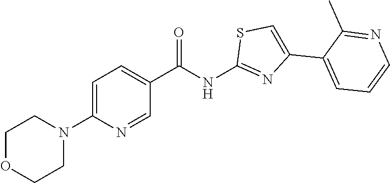 [SMILES: Cc1ncccc1-c1csc(NC(=O)c2ccc(N3CCOCC3)nc2)n1]

Compound N-[4-(2-methyl-3-pyridyl)thiazol-2-yl]-6-morpholino-pyridine-3-carboxamide was prepared from 4-(2-methylpyridin-3-yl)thiazol-2-amine and 6-morpholinonicotinic acid following a similar procedure to that described for the synthesis of N-[4-(2-chlorophenyl)thiazol-2-yl]-2-morpholino-pyrimidine-5-carboxamide except the compound was isolated by trituration from ethyl acetate to afford N-[4-(2-methyl-3-pyridyl)thiazol-2-yl]-6-morpholino-pyridine-3-carboxamide as a brown powder.

Yield 31 mg (19%). ¹H NMR (400 MHz, DMSO) δ 12.59 (br s, 1H), 8.94 (br s, 1H), 8.50 (d, J=4.4 Hz, 1H), 8.30 (d, J=9.6 Hz, 1H), 8.05 (d, J=6.9 Hz, 1H), 7.49 (br s, 1H), 7.37 (dd, J=4.4, 6.9 Hz, 1H), 6.99 (d, J=9.6 Hz, 1H), 3.80-3.65 (m, 8H), 2.72 (s, 3H). m/z: [ESI⁺] 382 (M+H)⁺, (C₁₉H₁₉N₅O₂S).

### Synthesis of N-[4-(2-methyl-3-pyridyl)thiazol-2-yl]-2-morpholino-pyrimidine-5-carboxamide (Compound 374)

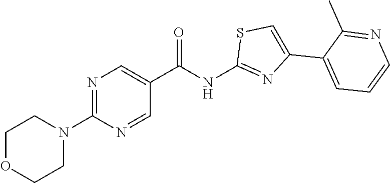 [SMILES: Cc1ncccc1-c1csc(NC(=O)c2cnc(N3CCOCC3)nc2)n1]

Compound N-[4-(2-methyl-3-pyridyl)thiazol-2-yl]-2-morpholino-pyrimidine-5-carboxamide was prepared from 4-(2-methylpyridin-3-yl)thiazol-2-amine and 2-morpholinopyrimidine-5-carboxylic acid following a similar procedure to that described for the synthesis of N-[4-(2-chlorophenyl)thiazol-2-yl]-2-morpholino-pyrimidine-5-carboxamide except the compound was isolated by trituration from ethyl acetate to afford N-[4-(2-methyl-3-pyridyl)thiazol-2-yl]-2-morpholino-pyrimidine-5-carboxamide as a beige solid.

Yield 75 mg (47%). ¹H NMR (400 MHz, DMSO) δ 12.72 (br s, 1H), 9.09 (s, 2H), 8.50 (dd, J=1.8, 4.8 Hz, 1H), 8.03 (dd, J=1.8, 7.7 Hz, 1H), 7.51 (s, 1H), 7.37 (dd, J=4.8, 7.7 Hz, 1H), 3.93-3.86 (m, 4H), 3.77-3.70 (m, 4H), 2.71 (s, 3H). m/z: [ESI⁺] 383 (M+H)⁺, (C₁₈H₁₈N₆O₂S).

### Synthesis of N-(4-(2-chlorophenyl)thiazol-2-yl)-4-(4-(3-methoxypropanoyl)piperazin-1-yl)benzamide (Compound 376)

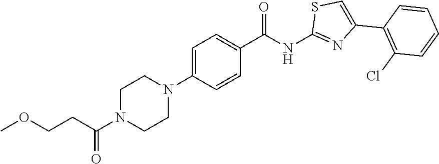 [SMILES: COCCC(=O)N1CCN(c2ccc(C(=O)Nc3nc(-c4ccccc4Cl)cs3)cc2)CC1]

To a solution of N-[4-(2-chlorophenyl)-1,3-thiazol-2-yl]-4-(piperazin-1-yl)benzamide (80 mg, 0.201 mmol) in DMF (2 ml) were added 3-methoxypropanoic acid (27 mg, 0.259 mmol), HATU (114 mg, 0.300 mmol) and DIPEA (0.10 mL, 0.605 mmol) at room temperature under a nitrogen atmosphere. The resulting mixture was stirred for 2 h at room temperature under a nitrogen atmosphere. The resulting mixture was purified by reverse phase flash chromatography with the following conditions: Column: Spherical C18, 20-40 um, 120 g; Mobile Phase A: water (plus 10 mM NH₄HCO3); Mobile Phase B: ACN; How rate: 60 mL/min; Gradient: 55% B-75% B in 20 min; Detector: UV 254/220 nm. The fractions containing desired product were collected and concentrated under reduced pressure to afford N-(4-(2-chlorophenyl)thiazol-2-yl)-4-(4-(3-methoxypropanoyl)piperazin-1-yl)benzamide as an off-white solid.

Yield 39 mg (40%). ¹H NMR (400 MHz, DMSO) δ 12.47 (br s, 1H), 8.06 (d, J=9.2 Hz, 2H), 7.92 (dd, J=1.9, 7.7 Hz, 1H), 7.61 (s, 1H), 7.56 (dd, J=1.2, 7.6 Hz, 1H), 7.48-7.35 (m, 2H), 7.03 (d, J=9.2 Hz, 2H), 3.62-3.56 (m, 6H), 3.41-3.34 (m, 4H), 3.24 (s, 3H), 2.63 (t, J=6.6 Hz, 2H). m/z: [ESI⁺] 485, 487 (M+H)⁺, (C₂₄H₂₅ClN₄O₃S).

### Synthesis of N-(4-(2-chlorophenyl)thiazol-2-yl)-4-(4-(2-methoxyethyl)piperazin-1-yl)benzamide (Compound 377)

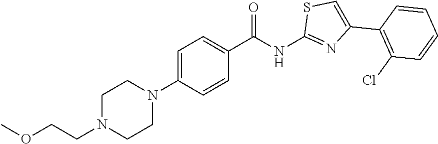 [SMILES: COCCN1CCN(c2ccc(C(=O)Nc3nc(-c4ccccc4Cl)cs3)cc2)CC1]

To a solution of N-[4-(2-chlorophenyl)-1,3-thiazol-2-yl]-4-(piperazin-1-yl)benzamide (200 mg, 0.501 mmol) in DMF (3 mL) were added potassium carbonate (139 mg, 1.006 mmol), potassium iodide (83 mg, 0.500 mmol) and 2-bromoethyl methyl ether (70 mg, 0.504 mmol) under a nitrogen atmosphere. The resulting mixture was stirred for 2 h at 80° C. under a nitrogen atmosphere The resulting mixture was cooled down to room temperature and purified by reverse phase flash chromatography with the following conditions: Column: Spherical C18, 20-40 um, 330 g; Mobile Phase A: water (plus 10 mM NH₄HCO3); Mobile Phase B: ACN; Flow rate: 80 mL/min; Gradient: 75% B-95% B in 20 min; Detector: UV 254/220 nm. The fractions containing desired product were collected, concentrated and lyophilized to afford N-[4-(2-chlorophenyl)-1,3-thiazol-2-yl]-4-[4-(2-methoxyethyl)piperazin-1-yl]benzamide as an off-white solid.

Yield 165 mg (72%). ¹H NMR (400 MHz, DMSO) δ 12.45 (br s, 1H), 8.04 (d, J=8.8 Hz, 2H), 7.92 (dd, J=1.9, 7.7 Hz, 11H), 7.62 (s, 11H), 7.57 (dd, J=1.4, 7.9 Hz, 11H), 7.45 (td, J=1.5, 7.5 Hz, 11H), 7.39 (td, J=1.9, 7.6 Hz, 1H), 7.01 (d, J=8.8 Hz, 2H), 3.47 (t, J=5.7 Hz, 2H), 3.33-3.29 (m, 4H), 3.25 (s, 3H), 2.58-2.48 (m, 6H). m/z: [ESI⁺] 457, 459 (M+H)⁺, (C₂₃H₂₅ClN₄O₂S).

### Synthesis of N-(4-(2-(2-methoxyethoxy)phenyl)thiazol-2-yl)-4-morpholinobenzamide (Compound 378)

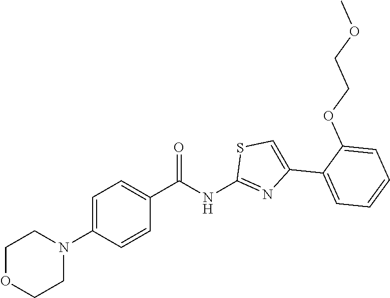 [SMILES: COCCOc1ccccc1-c1csc(NC(=O)c2ccc(N3CCOCC3)cc2)n1]

To a solution of N-(4-bromo-1,3-thiazol-2-yl)-4-(morpholin-4-yl)benzamide (50 mg, 0.136 mmol) in 1,4-dioxane (5.4 mL) were added water (0.6 mL), 2-(2-methoxyethoxy)phenylboronic acid (57 mg, 0.291 mmol), cesium carbonate (111 mg, 0.341 mmol) and tetrakis(triphenylphosphine)palladium(0) (16 mg, 0.014 mmol) at room temperature under an argon atmosphere. The resulting mixture was stirred at 100° C. for 2 h under an argon atmosphere. After cooling down to room temperature, the resulting mixture was concentrated under reduced pressure. The residue was purified by Prep-HPLC with the following conditions (Column: XBridge Shield RP C18 OBD column, 30×150 mm, 5 um; Mobile Phase A: water (plus 0.05% TFA), Mobile Phase B: ACN; Flow rate: 20 mL/min; Gradient: 30% B to 55% B in 8 min; Detector: UV 220/254 nm. Desired fractions were collected, concentrated and lyophilized to afford N-(4-(2-(2-methoxyethoxy)phenyl)thiazol-2-yl)-4-morpholinobenzamide as a white solid.

Yield 5.5 mg (9%). ¹H NMR (400 MHz, DMSO) δ 12.35 (br s, 1H), 8.21 (dd, J=1.8, 7.7 Hz, 1H), 8.07 (d, J=8.6 Hz, 2H), 7.77 (s, 1H), 7.30 (t, J=7.4 Hz, 1H), 7.14 (d, J=8.2 Hz, 1H), 7.10-7.01 (m, 3H), 4.26 (t, J=4.6 Hz, 2H), 3.85-3.78 (m, 2H), 3.76-3.70 (m, 4H), 3.38 (s, 3H), 3.35-3.33 (m, 4H). m/z: [ESI⁺] 440 (M+H)⁺, (C₂₃H₂₅N₃O₄S).

### Synthesis of N-(4-(2-((2-methoxyethoxy)methyl)phenyl)thiazol-2-yl)-4-morpholinobenzamide (Compound 379)

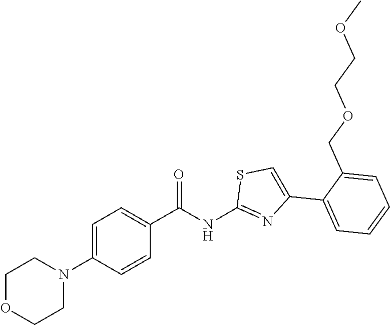 [SMILES: COCCOCc1ccccc1-c1csc(NC(=O)c2ccc(N3CCOCC3)cc2)n1]

To a solution of N-(4-bromo-1,3-thiazol-2-yl)-4-(morpholin-4-yl)benzamide (50 mg, 0.136 mmol) in 1,4-dioxane (5.4 mL) were added water (0.6 mL), 2-[(2-methoxyethoxy)methyl]phenylboronic acid (57 mg, 0.271 mmol), cesium carbonate (111 mg, 0.341 mmol) and tetrakis(triphenylphosphine)palladium(0) (16 mg, 0.014 mmol) at room temperature under an argon atmosphere. After being stirred at 100° C. for 2 h under an argon atmosphere, the mixture was cooled down to room temperature. The resulting mixture was concentrated under reduced pressure. The residue was purified by Prep-HPLC with the following conditions (Column: SunFire Prep C18 OBD Column, 19×150 mm, 5 um; Mobile Phase A: water (10 mmol/L NH₄HCO3), Mobile Phase B: ACN; Flow rate: 25 mL/min; Gradient: 54% B to 72% B in 8 min; Detector: UV 254/220 nm). Desired fractions were collected, concentrated and lyopjhlized to afford N-(4-[2-[(2-methoxyethoxy)methyl]phenyl]-1,3-thiazol-2-yl)-4-(morpholin-4-yl)benzamide as a white solid.

Yield 5.5 mg (9%). ¹H NMR (400 MHz, CDCl₃) δ 8.14 (d, J=9.2 Hz, 2H), 7.63 (t, J=5.6 Hz, 1H), 7.55-7.45 (m, 4H), 6.98 (d, J=9.2 Hz, 2H), 4.57 (s, 2H), 3.93-3.86 (m, 4H), 3.69 (t, J=7.2 Hz, 2H), 3.57 (t, J=7.2 Hz, 2H), 3.43-3.35 (m, 7H), NH amide proton not visible.

¹H NMR (400 MHz, DMSO) δ 12.42 (br s, 1H), 8.06 (d, J=9.2 Hz, 2H), 7.72 (dd, J=2.4, 7.6 Hz, 1H), 7.53 (t, J=6.4 Hz, 1H), 7.42-7.36 (m, 3H), 7.04 (d, J=9.2 Hz, 2H), 4.64 (s, 2H), 3.75 (t, J=4.4 Hz, 4H), 3.63-3.27 (m, 8H), 3.26 (s, 3H). m/z: [ESI⁺] 454 (M+H)⁺, (C₂₄H₂₇N₃O₄S).

### Synthesis of N-(4-(2-chlorophenyl)thiazol-2-yl)-N-methyl-4-morpholinobenzamide (Compound 399)

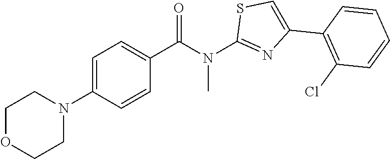 [SMILES: CN(C(=O)c1ccc(N2CCOCC2)cc1)c1nc(-c2ccccc2Cl)cs1]

To a stirred solution of N-[4-(2-chlorophenyl)thiazol-2-yl]-4-morpholino-benzamide (100 mg, 0.25 mmol) in DMF (3 ml) was added sodium hydride (60% dispersion in mineral oil, 20 mg, 0.50 mmol) portionwise at 0° C. under a nitrogen atmosphere and the mixture was stirred for 30 min at 0° C. To the resulting mixture was added iodomethane (36 mg, 0.25 mmol) dropwise at 0° C. The reaction was stirred for 16 h at room temperature. The reaction was quenched with acetic acid (1 drop) and purified by reverse phase flash chromatography with the following conditions: column, C18 silica gel; mobile phase: acetonitrile in water (plus 10 mmol/L NH₄HCO₃), 40% to 60% gradient in 10 min; detector: UV 220/254 nm. The fractions containing the desired product were collected, concentrated under reduced pressure and lyophilized to afford N-(4-(2-chlorophenyl)thiazol-2-yl)-N-methyl-4-morpholinobenzamide as an off-white solid.

Yield 4 mg (4%). ¹H NMR (400 MHz, DMSO) δ 7.96 (dd, J=1.6, 7.6 Hz, 1H), 7.74 (s, 1H), 7.64-7.54 (m, 3H), 7.49-7.43 (m, 1H), 7.42-7.36 (m, 1H), 7.07-7.03 (m, 2H), 3.80-3.73 (m, 4H), 3.70 (s, 3H), 3.31-3.25 (m, 4H). m/z: [ESI⁺] 414, 416 (M+H)⁺, (C₂₁H₂₀ClN₃O₂S).

### Synthesis of N-(4-(2-chlorophenyl)thiazol-2-yl)-N-methyl-5-morpholinopicolinamide (Compound 400)

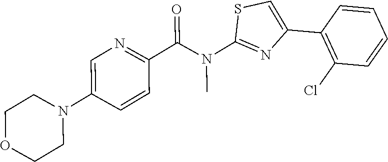 [SMILES: CN(C(=O)c1ccc(N2CCOCC2)cn1)c1nc(-c2ccccc2Cl)cs1]

Compound N-(4-(2-chlorophenyl)thiazol-2-yl)-N-methyl-5-morpholinopicolinamide was prepared from N-[4-(2-chlorophenyl)thiazol-2-yl]-5-morpholino-pyridine-2-carboxamide (60 mg, 0.15 mmol), following a procedure similar to that described for the synthesis of N-(4-(2-chlorophenyl)thiazol-2-yl)-N-methyl-4-morpholinobenzamide and was isolated as a yellow solid.

Yield 21 mg (34%). ¹H NMR (400 MHz, DMSO) δ 8.43 (d, J=2.8 Hz, 1H), 8.23 (d, J=8.8 Hz, 1H), 7.71 (dd, J=1.6, 8.0 Hz, 1H), 7.67-7.58 (m, 2H), 7.58-7.50 (m, 1H), 7.37 (dd, J=2.8, 8.8 Hz, 1H), 7.09 (s, 1H), 3.81-3.74 (m, 4H), 3.52 (s, 3H), 3.33-3.29 (m, 4H). m/z: [ESI⁺] 415, 417 (M+H)⁺, (C₂₀H₁₉ClN₄O₂S).

### Synthesis of 4-(4-(2-(3-(but-3-yn-1-yl)-3H-diazirin-3-yl)ethyl)piperazin-1-yl)-N-(4-(2-chlorophenyl)thiazol-2-yl)benzamide hemiformate (Compound 484)

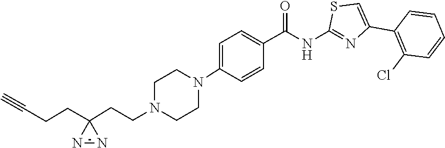 [SMILES: C#CCCC1(CCN2CCN(c3ccc(C(=O)Nc4nc(-c5ccccc5Cl)cs4)cc3)CC2)N=N1]

To a stirred solution of N-[4-(2-chlorophenyl)thiazol-2-yl]-4-piperazin-1-yl-benzamide (133 mg, 0.333 mmol) and DIPEA (117 mg, 0.91 mmol) in DMF (5 ml) was added 3-(but-3-yn-1-yl)-3-(2-iodoethyl)-3H-diazirine (75 mg, 0.30 mmol), dropwise at room temperature under a nitrogen atmosphere. The resulting solution was stirred in the dark, for 16 h at 60° C. After cooling to room temperature, the resulting mixture was purified by Prep-HPLC with the following conditions: Column: Spherical C18, 20-40 μm, 120 g; Mobile Phase A: water (plus 10 mmol/L HCOOH); Mobile Phase B: acitonitrile; Flow rate: 45 mL/min; Gradient: 35%-50% B in 12 min; Detector: UV 254/220 nm. The fractions containing the desired product were collected, concentrated under reduced pressure and lyophilized to afford 4-(4-(2-(3-(but-3-yn-1-yl)-3H-diazirin-3-yl)ethyl)piperazin-1-yl)-N-(4-(2-chlorophenyl)thiazol-2-yl)benzanide hemiformate as an off-white solid.

Yield 69 mg (44%). ¹H NMR (300 MHz, DMSO) δ 12.46 (br s, 1H), 8.16 (s, 0.53H, HCOOH), 8.08-8.00 (m, 2H), 7.96-7.88 (m, 1H), 7.66-7.63 (m, 1H), 7.61-7.56 (m, 1H), 7.52-7.33 (m, 2H), 7.03 (d, J=8.4 Hz, 2H), 3.38-3.31 (m, 6H), 2.91-2.81 (m, 1H), 2.50-2.46 (m, 2H), 2.21 (t, J=7.2 Hz, 2H), 2.04 (dt, J=2.8, 7.2 Hz, 2H), 1.62 (dt, J=3.6, 7.2 Hz, 4H). m/z: [ESI⁺] 519, 521 (M+H)⁺, (C₂₇H₂₇ClN₆OS).

### Synthesis of N-(4-(2-chlorophenyl)thiazol-2-yl)-5-(hexahydropyrrolo[1,2-a]pyrazin-2(1H)-yl)picolinamide (Compound 461)

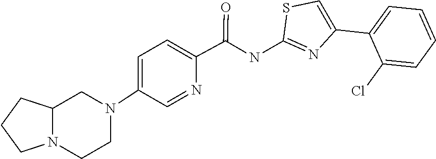

To a solution of N-(4-(2-chlorophenyl)thiazol-2-yl)-5-fluoropicolinamide (150 mg, 0.449 mmol) and octahydropyrrolo[1,2-a]pyrazine (85 mg, 0.67 mmol) in DMF (2 mL) was added DIPEA (174 mg, 1.35 mmol) dropwise at room temperature, under a nitrogen atmosphere. The resulting solution was stirred for 5 h at 100° C., under a nitrogen atmosphere. After cooling to room temperature, the resulting mixture was purified by Prep-HPLC with the following conditions: Column: XBridge Shield RP18 OBD Column, 30×150 mm, 5 μm; Mobile Phase A: water (plus 10 mmol/L NH₄HCO₃); Mobile Phase B: acetonitrile; Flow rate: 60 mL/min; Gradient: 60% to 80% B in 8 min; Detector: UV 254/220 nm. The fractions containing the desired product were collected, concentrated under reduced pressure and lyophilized to afford N-(4-(2-chlorophenyl)thiazol-2-yl)-5-(hexahydropyrrolo[1,2-a]pyrazin-2(1H)-yl)picolinamide as an off-white solid.

Yield 82 mg (42%). ¹H NMR (400 MHz, DMSO) δ 11.59 (br s, 1H), 8.43 (d, J=2.8 Hz, 1H), 8.00 (d, J=8.8 Hz, 1H), 7.93 (dd, J=2.0, 7.6 Hz, 1H), 7.71 (s, 1H), 7.57 (dd, J=2.0, 7.6 Hz, 1H), 7.50 (dd, J=2.8, 8.8 Hz, 1H), 7.47-7.36 (m, 2H), 4.13 (dt, J=2.8, 11.6 Hz, 1H), 3.97 (d, J=12.0 Hz, 1H), 3.14-3.01 (m, 2H), 3.00-2.92 (m, 1H), 2.63 (t, J=11.2 Hz, 1H), 2.24 (t, J=11.2 Hz, 1H), 2.15-1.96 (m, 2H), 1.93-1.81 (m, 1H), 1.81-1.61 (m, 2H), 1.50-1.31 (m, 1H). m/z: [ESI⁺] 440, 442 (M+H)⁺, (C₂₂H₂₂ClN₅OS).

### Synthesis of (R)—N-(4-(2-chlorophenyl)thiazol-2-yl)-5-(3-hydroxypiperidin-1-yl)picolinamide (Compound 487)

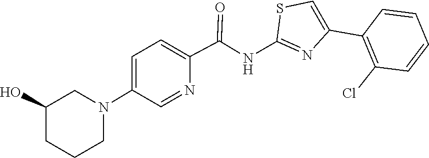 [SMILES: O=C(Nc1nc(-c2ccccc2Cl)cs1)c1ccc(N2CCC[C@@H](O)C2)cn1]

Compound (R)—N-(4-(2-chlorophenyl)thiazol-2-yl)-5-(3-hydroxypiperidin-1-yl)picolinamide was prepared from N-(4-(2-chlorophenyl)thiazol-2-yl)-5-fluoropicolinamide (0.40 g, 1.20 mmol) and (R)-piperidin-3-ol (0.18 g, 1.78 mmol), following a procedure similar to that described for the synthesis of N-(4-(2-chlorophenyl)thiazol-2-yl)-5-(hexahydropyrrolo[1,2-a]pyrazin-2(1H)-yl)picolinamide and was isolated as an off-white solid.

Yield 240 mg (48%). ¹H NMR (400 MHz, DMSO) δ 511.54 (br s, 1H), 8.37 (d, J=2.8 Hz, 1H), 7.97 (d, J=8.8 Hz, 1H), 7.93 (dd, J=2.0, 8.0 Hz, 1H), 7.71 (s, 1H), 7.57 (dd, J=1.6, 7.6 Hz, 1H), 7.47-7.36 (m, 3H), 4.93 (d, J=4.4 Hz, 1H), 3.79 (dd, J=3.6, 12.8 Hz, 1H), 3.75-3.68 (m, 1H), 3.67-3.56 (m, 1H), 3.15-3.04 (m, 1H), 2.97 (dd, J=8.4, 12.8 Hz, 1H), 1.95-1.85 (m, 1H), 1.84-1.76 (m, 1H), 1.58-1.36 (m, 2H). m/z: [ESI⁺] 415, 417 (M+H)⁺, (C₂₀H₁₉ClN₄O₂S).

### Synthesis of (S)—N-(4-(2-chlorophenyl)thiazol-2-yl)-5-(3-hydroxypiperidin-1-yl)picolinamide (Compound 488)

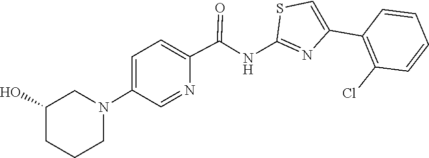 [SMILES: O=C(Nc1nc(-c2ccccc2Cl)cs1)c1ccc(N2CCC[C@H](O)C2)cn1]

Compound (S)—N-(4-(2-chlorophenyl)thiazol-2-yl)-5-(3-hydroxypiperidin-1-yl)picolinamide was prepared from N-(4-(2-chlorophenyl)thiazol-2-yl)-5-fluoropicolinamide (0.40 g, 1.20 mmol) and (S)-piperidin-3-ol (0.18 g, 1.78 mmol), following a procedure similar to that described for the synthesis of N-(4-(2-chlorophenyl)thiazol-2-yl)-5-(hexahydropyrrolo[1,2-a]pyrazin-2(1H)-yl)picolinamide and was isolated as an off-white solid.

Yield 380 mg (76%). ¹H NMR (400 MHz, DMSO) δ 11.55 (br s, 1H), 8.37 (d, J=2.8 Hz, 1H), 7.97 (d, J=8.8 Hz, 1H), 7.93 (dd, J=2.0, 8.0 Hz, 1H), 7.71 (s, 1H), 7.57 (dd, J=1.6, 7.6 Hz, 1H), 7.50-7.34 (m, 3H), 4.93 (d, J=4.4 Hz, 1H), 3.79 (dd, J=3.6, 12.8 Hz, 1H), 3.75-3.68 (m, 1H), 3.67-3.58 (m, 1H), 3.15-3.05 (m, 1H), 2.97 (dd, J=8.4, 12.8 Hz, 1H), 1.97-1.88 (m, 1H), 1.84-1.74 (m, 1H), 1.61-1.36 (m, 2H). m/z: [ESI⁺] 415, 417 (M+H)⁺, (C₂₀H₁₉ClN₄O₂S).

### Synthesis of N-(4-(2-chlorophenyl)thiazol-2-yl)-5-(4-(2-hydroxyethyl)piperidin-1-yl)picolinamide (Compound 478)

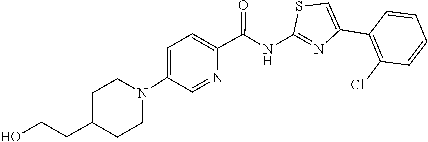 [SMILES: O=C(Nc1nc(-c2ccccc2Cl)cs1)c1ccc(N2CCC(CCO)CC2)cn1]

Compound N-(4-(2-chlorophenyl)thiazol-2-yl)-5-(4-(2-hydroxyethyl)piperidin-1-yl)picolinamide was prepared from N-(4-(2-chlorophenyl)thiazol-2-yl)-5-fluoropicolinamide (0.50 g, 1.50 mmol) and 2-(piperidin-4-yl)ethan-1-ol (0.21 g, 1.63 mmol), following a procedure similar to that described for the synthesis of N-(4-(2-chlorophenyl)thiazol-2-yl)-5-(hexahydropyrrolo[1,2-a]pyrazin-2(1H)-yl)picolinamide and was isolated as an off-white solid.

Yield 0.45 g (68%). ¹H NMR (400 MHz, DMSO) δ 11.50 (br s, 1H), 8.35 (d, J=2.8 Hz, 1H), 8.02-7.88 (m, 2H), 7.70 (s, 1H), 7.55 (dd, J=1.6, 8.0 Hz, 1H), 7.46-7.33 (m, 3H), 4.40 (t, J=5.2 Hz, 1H), 3.97 (d, J=12.8 Hz, 2H), 3.50-3.43 (m, 2H), 2.86 (dt, J=2.8, 12.8 Hz, 2H), 1.74 (dd, J=3.2, 13.6 Hz, 2H), 1.70-1.55 (m, 1H), 1.44-1.34 (m, 2H), 1.29-1.09 (m, 2H). m/z: [ESI⁺] 443, 445 (M+H)⁺, (C₂₂H₂₃CN₄O₂S).

### Synthesis of N-(4-(2-chlorophenyl)thiazol-2-yl)-5-(4-(hydroxymethyl)piperidin-1-yl)picolinamide (Compound 479)

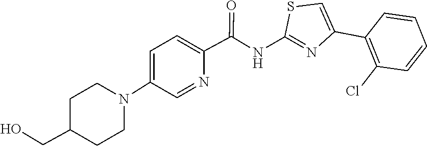 [SMILES: O=C(Nc1nc(-c2ccccc2Cl)cs1)c1ccc(N2CCC(CO)CC2)cn1]

Compound N-(4-(2-chlorophenyl)thiazol-2-yl)-5-(4-(hydroxymethyl)piperidin-1-yl)picolinamide was prepared from N-(4-(2-chlorophenyl)thiazol-2-yl)-5-fluoropicolinamide (0.50 g, 1.50 mmol) and piperidin-4-ylmethanol (0.19 g, 1.65 mmol), following a procedure similar to that described for the synthesis of N-(4-(2-chlorophenyl)thiazol-2-yl)-5-(hexahydropyrrolo[1,2-a]pyrazin-2(1H)-yl)picolinamide and was isolated as an off-white solid.

Yield 0.22 g (34%). ¹H NMR (400 MHz, DMSO) δ 11.53 (br s, 1H), 8.38 (d, J=2.8 Hz, 1H), 7.97 (d, J=8.8 Hz, 1H), 7.93 (dd, J=2.0, 8.0 Hz, 1H), 7.70 (s, 1H), 7.56 (dd, J=1.6, 7.6 Hz, 1H), 7.49-7.33 (m, 3H), 4.53 (t, J=5.2 Hz, 1H), 4.22-3.88 (m, 2H), 3.29 (t, J=5.6 Hz, 2H), 2.91 (dt, J=2.8, 12.8 Hz, 2H), 1.79-1.70 (m, 2H), 1.71-1.59 (m, 1H), 1.29-1.10 (m, 2H). m/z: [ESI⁺] 429, 431 (M+H)⁺, (C₂₁H₂₁ClN₄O₂S).

### Synthesis of N-(4-(2-chlorophenyl)thiazol-2-yl)-5-(4-methylpiperazine-1-carbonyl)picolinamide (Compound 437)

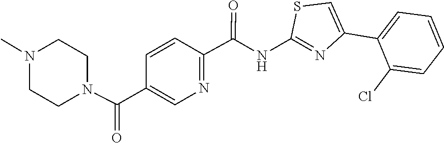 [SMILES: CN1CCN(C(=O)c2ccc(C(=O)Nc3nc(-c4ccccc4Cl)cs3)nc2)CC1]

Compound N-(4-(2-chlorophenyl)thiazol-2-yl)-5-(4-methylpiperazine-1-carbonyl)picolinamide was prepared from 5-bromo-N-(4-(2-chlorophenyl)thiazol-2-yl)picolinamide (100 mg, 0.25 mmol) and 1-methylpiperazine (63 mg, 0.63 mmol), following a procedure similar to that described for the synthesis of tert-butyl 4-(6-((4-(2-chlorophenyl)thiazol-2-yl)carbamoyl)nicotinoyl)piperazine-1-carboxylate and was isolated as an off-white solid.

Yield 20 mg (18%). ¹H NMR (400 MHz, DMSO) δ 12.28 (br s, 1H), 8.79 (dd, J=0.8, 2.0 Hz, 1H), 8.25 (dd, J=0.8, 8.0 Hz, 1H), 8.13 (dd, J=2.0, 8.0 Hz, 1H), 7.93 (dd, J=2.0, 7.6 Hz, 1H), 7.78 (s, 1H), 7.58 (dd, J=1.6, 8.0 Hz, 1H), 7.54-7.32 (m, 2H), 3.72-3.62 (m, 2H), 3.32-3.32 (m, 2H), 2.46-2.28 (m, 4H), 2.23 (s, 311). m/z: [ESI⁺] 442, 444 (M+H)⁺, (C₂₁H₂₀ClN₅O₂S)

### Synthesis of N-(4-(2-chlorophenyl)thiazol-2-yl)-5-(3-hydroxypiperidin-1-yl)picolinamide (Compound 477)

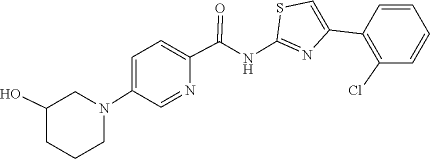 [SMILES: O=C(Nc1nc(-c2ccccc2Cl)cs1)c1ccc(N2CCCC(O)C2)cn1]

Compound N-(4-(2-chlorophenyl)thiazol-2-yl)-5-(3-hydroxypiperidin-1-yl)picolinamide was prepared from 5-bromo-N-(4-(2-chlorophenyl)thiazol-2-yl)picolinamide (150 mg, 0.38 mmol) and piperidin-3-ol (58 mg, 0.57 mmol), following a procedure similar to that described for the synthesis of methyl 5-(2-methyl-1-oxo-2,8-diazaspiro[4.5]decan-8-yl)picolinate and was isolated as an off-white solid.

Yield 12 mg (8%). ¹H NMR (400 MHz, DMSO) δ 11.54 (br s, 1H), 8.37 (d, J=2.8 Hz, 1H), 7.97 (d, J=8.8 Hz, 1H), 7.93 (dd, J=2.0, 7.6 Hz, 1H), 7.71 (s, 1H), 7.61-7.53 (m, 1H), 7.48-7.35 (m, 3H), 4.93 (d, J=4.4 Hz, 1H), 3.79 (dd, J=4.0, 12.4 Hz, 1H), 3.75-3.67 (m, 1H), 3.66-3.56 (m, 1H), 3.15-3.01 (m, 1H), 2.97 (dd, J=8.4, 12.8 Hz, 1H), 1.96-1.85 (m, 1H), 1.85-1.74 (m, 1H), 1.59-1.37 (m, 2H). m/z: [ESI⁺] 415, 417 (M+H)⁺, (C₂₀H₁₉ClN₄O₂S)

### Synthesis of N-(4-(2-chlorophenyl)thiazol-2-yl)-5-(pyrrolidin-1-yl)picolinamide (Compound 481)

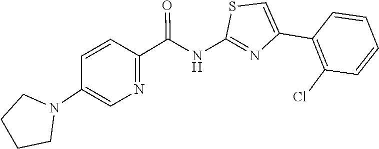 [SMILES: O=C(Nc1nc(-c2ccccc2Cl)cs1)c1ccc(N2CCCC2)cn1]

Compound N-(4-(2-chlorophenyl)thiazol-2-yl)-5-(pyrrolidin-1-yl)picolinamide was prepared from 5-bromo-N-(4-(2-chlorophenyl)thiazol-2-yl)picolinamide (150 mg, 0.38 mmol) and pyrrolidine (40 mg, 0.56 mmol), following a procedure similar to that described for the synthesis of methyl 5-(2-methyl-1-oxo-2,8-diazaspiro[4.5]decan-8-yl)picolinate and was isolated as a grey solid.

Yield 13 mg (9%). ¹H NMR (400 MHz, DMSO) δ 11.46 (br s, 1H), 8.04 (d, J=2.8 Hz, 1H), 7.99 (d, J=8.8 Hz, 1H), 7.93 (dd, J=2.0, 7.6 Hz, 1H), 7.69 (s, 1H), 7.57 (d, J=7.6 Hz, 1H), 7.50-7.33 (m, 2H), 7.06 (dd, J=2.8, 8.8 Hz, 1H), 3.45-3.38 (m, 4H), 2.06-1.93 (m, 4H). m/z: [ESI⁺] 385, 387 (M+H)⁺, (C₁₉H₁₇ClN₄OS)

### Synthesis of (S)—N-(4-(2-chlorophenyl)thiazol-2-yl)-5-(3-methylmorpholino)picolinamide (Compound 464)

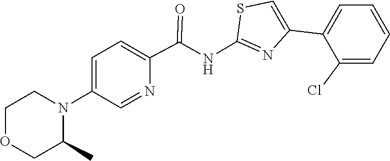 [SMILES: C[C@H]1COCCN1c1ccc(C(=O)Nc2nc(-c3ccccc3Cl)cs2)nc1]

Compound (S)—N-(4-(2-chlorophenyl)thiazol-2-yl)-5-(3-methylmorpholino)picolinamide was prepared from 5-bromo-N-(4-(2-chlorophenyl)thiazol-2-yl)picolinamide (100 mg, 0.25 mmol) and (S)-3-methylmorpholine (38 mg, 0.38 mmol), following a procedure similar to that described for the synthesis of methyl 5-(2-methyl-1-oxo-2,8-diazaspiro[4.5]decan-8-yl)picolinate and was isolated as an off-white solid.

Yield 5 mg (5%). ¹H NMR (400 MHz, DMSO) δ 11.62 (br s, 1H), 8.37 (d, J=2.8 Hz, 1H), 8.03 (d, J=8.8 Hz, 1H), 7.93 (dd, J=2.0, 7.6 Hz, 1H), 7.71 (s, 1H), 7.57 (dd, J=1.6, 8.0 Hz, 1H), 7.50-7.42 (m, 2H), 7.42-7.37 (m, 1H), 4.26-4.12 (m, 1H), 4.07-3.92 (m, 1H), 3.82-3.67 (m, 2H), 3.67-3.52 (m, 2H), 3.21-3.09 (m, 1H), 1.15 (d, J=6.8 Hz, 3H). m/z: [ESI⁺] 415, 417 (M+H)⁺, (C₂₀H₁₉ClN₄O₂S)

### Synthesis of 5-(4-acetylpiperazin-1-yl)-N-(4-(2-chlorophenyl)thiazol-2-yl)-3-methylpicolinamide (Compound 458)

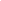

Compound 5-(4-acetylpiperazin-1-yl)-N-(4-(2-chlorophenyl)thiazol-2-yl)-3-methylpicolinamide was prepared from 5-bromo-N-(4-(2-chlorophenyl)thiazol-2-yl)-3-methylpicolinamide (200 mg, 0.49 mmol) and 1-(piperazin-1-yl)ethan-1-one (94 mg, 0.73 mmol), following a procedure similar to that described for the synthesis of methyl 5-(2-methyl-1-oxo-2,8-diazaspiro[4.5]decan-8-yl)picolinate and was isolated as an off-white solid.

Yield 16 mg (7%). ¹H NMR (400 MHz, DMSO) δ 11.64 (br s, 1H), 8.30 (s, 1H), 7.93 (dd, J=2.0, 7.6 Hz, 1H), 7.70 (s, 1H), 7.57 (dd, J=1.6, 7.6 Hz, 1H), 7.48-7.36 (m, 2H), 7.30 (d, J=2.4 Hz, 1H), 3.68-3.56 (m, 4H), 3.53-3.49 (m, 2H), 3.49-3.41 (m, 2H), 2.65 (s, 3H), 2.07 (s, 3H). m/z: [ESI⁺] 456, 458 (M+H)⁺, (C₂₂H₂₂ClN₅O₂S)

### Synthesis of 4-(4-acetylpiperazin-1-yl)-N-(4-(2-chlorophenyl)thiazol-2-yl)-2-methylbenzamide (Compound 473)

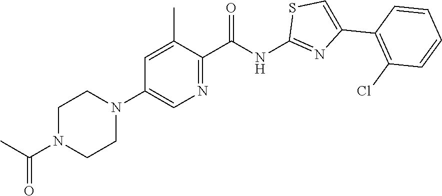 [SMILES: CC(=O)N1CCN(c2cnc(C(=O)Nc3nc(-c4ccccc4Cl)cs3)c(C)c2)CC1]

Compound 4-(4-acetylpiperazin-1-yl)-N-(4-(2-chlorophenyl)thiazol-2-yl)-2-methylbenzamide was prepared from 4-bromo-N-(4-(2-chlorophenyl)thiazol-2-yl)-2-methylbenzamide (300 mg, 0.74 mmol) and 1-(piperazin-1-yl)ethan-1-one (142 mg, 1.11 mmol), following a procedure similar to that described for the synthesis of methyl 5-(2-methyl-1-oxo-2,8-diazaspiro[4.5]decan-8-yl)picolinate and was isolated as an off-white solid.

Yield 16 mg (5%). ¹H NMR (400 MHz, DMSO) δ 12.38 (br s, 1H), 7.88 (dd, J=2.0, 7.6 Hz, 1H), 7.67-7.50 (m, 3H), 7.48-7.35 (m, 2H), 6.90-6.80 (m, 2H), 3.64-3.54 (M, 6H), 3.30-3.20 (m, 2H), 2.46 (s, 3H), 2.06 (s, 3H). m/z: [ESI⁺] 455, 457 (M+H)⁺, (C₂₃H₂₃ClN₄O₂S)

### Synthesis of 5-(4-acetylpiperazin-1-yl)-N-(4-(2-chlorophenyl)thiazol-2-yl)thiophene-2-carboxamide (Compound 463)

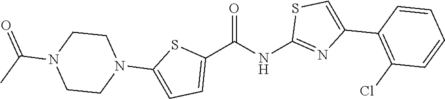 [SMILES: CC(=O)N1CCN(c2ccc(C(=O)Nc3nc(-c4ccccc4Cl)cs3)s2)CC1]

Compound 5-(4-acetylpiperazin-1-yl)-N-(4-(2-chlorophenyl)thiazol-2-yl)thiophene-2-carboxamide was prepared from 5-bromo-N-(4-(2-chlorophenyl)thiazol-2-yl)thiophene-2-carboxamide (500 mg, 1.25 mmol) and 1-(piperazin-1-yl)ethan-1-one (240 mg, 1.87 mmol), following a procedure similar to that described for the synthesis of methyl 5-(2-methyl-1-oxo-2,8-diazaspiro[4.5]decan-8-yl)picolinate and was isolated as a yellow solid.

Yield 48 mg (9%). ¹H NMR (400 MHz, DMSO) δ 12.45 (br s, 1H), 8.07 (d, J=4.4 Hz, 1H), 7.90 (dd, J=2.0, 8.0 Hz, 1H), 7.59 (s, 1H), 7.57 (dd, J=1.6, 8.0 Hz, 1H), 7.48-7.35 (m, 2H), 6.32 (d, J=4.4 Hz, 1H), 3.61 (t, J=5.2 Hz, 4H), 3.33-3.30 (m, 2H), 3.25 (t, J=5.2 Hz, 2H), 2.06 (s, 3H). m/z: [ESI⁺] 447, 449 (M+H)⁺, (C₂₀H₁₉ClN₄O₂S₂)

### Synthesis of 4-(4-acetylpiperazin-1-yl)-N-(4-(2-chlorophenyl)thiazol-2-yl)thiophene-2-carboxamide (Compound 462)

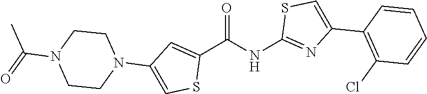 [SMILES: CC(=O)N1CCN(c2csc(C(=O)Nc3nc(-c4ccccc4Cl)cs3)c2)CC1]

Compound 4-(4-acetylpiperazin-1-yl)-N-(4-(2-chlorophenyl)thiazol-2-yl)thiophene-2-carboxamide was prepared from 4-bromo-N-(4-(2-chlorophenyl)thiazol-2-yl)thiophene-2-carboxamide (280 mg, 0.70 mmol) and 1-(piperazin-1-yl)ethan-1-one (135 mg, 1.05 mmol), following a procedure similar to that described for the synthesis of methyl 5-(2-methyl-1-oxo-2,8-diazaspiro[4.5]decan-8-yl)picolinate and was isolated as an off-white solid.

Yield 25 mg (8%). ¹H NMR (400 MHz, DMSO) δ 12.72 (br s, 1H), 8.24 (t, J=2.4 Hz, 1H), 7.90 (dd, J=2.0, 7.6 Hz, 1H), 7.68 (s, 1H), 7.58 (dd, J=1.6, 7.6 Hz, 1H), 7.50-7.34 (m, 2H), 6.89 (d, J=1.6 Hz, 1H), 3.65-3.55 (m, 4H), 3.11 (t, J=5.2 Hz, 2H), 3.05 (t, J=5.2 Hz, 2H), 2.06 (s, 3H). m/z: [ESI⁺] 447, 449 (M+H)⁺, (C₂₀H₁₉ClN₄O₂S₂)

### Synthesis of 5-(4-aminopiperidin-1-yl)-N-(4-(2-chlorophenyl)thiazol-2-yl)picolinamide (Compound 475)

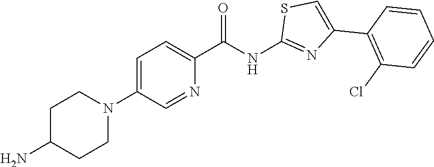 [SMILES: NC1CCN(c2ccc(C(=O)Nc3nc(-c4ccccc4Cl)cs3)nc2)CC1]

Compound 5-(4-aminopiperidin-1-yl)-N-(4-(2-chlorophenyl)thiazol-2-yl)picolinamide was prepared from tert-butyl (1-(6-((4-(2-chlorophenyl)thiazol-2-yl)carbamoyl)pyridin-3-yl)piperidin-4-yl)carbamate (120 mg, 0.23 mmol), following a procedure similar to that described for the synthesis of N-(4-(2-chlorophenyl)thiazol-2-yl)-5-(2,6-diazaspiro[3.3]heptan-2-yl)picolinamide 2,2,2-trifluoroacetate salt, except that it was purified by Prep-HPLC with the following conditions: Column: XBridge Shield RP18 OBD Column, 30×150 mm, 5 μm; Mobile Phase A: water (plus 10 mmol/L NH₄HCO₃); Mobile Phase B: acetonitrile; Flow rate: 60 mL/min; Gradient: 50% to 70% B in 9 min; Detector: UV 254/220 nm. The fractions containing the desired product were collected, concentrated under reduced pressure and lyophilized to afford 5-(4-aminopiperidin-1-yl)-N-(4-(2-chlorophenyl)thiazol-2-yl)picolinamide as an off-white solid.

Yield 22 mg (23%). ¹H NMR (400 MHz, DMSO) δ 11.63 (br s, 1H), 8.45 (d, J=2.8 Hz, 1H), 8.03 (d, J=8.8 Hz, 1H), 8.01-7.95 (m, 2H), 7.93 (dd, J=2.0, 7.6 Hz, 1H), 7.72 (s, 1H), 7.58 (dd, J=1.6, 8.0 Hz, 1H), 7.53 (dd, J=2.8, 8.8 Hz, 1H), 7.49-7.37 (m, 2H), 4.17-3.92 (m, 2H), 3.49-3.25 (m, 1H), 3.12-2.98 (m, 2H), 1.99 (dd, J=4.0, 13.2 Hz, 2H), 1.71-1.49 (m, 2H). Aliphatic NH₂ not observed. m/z: [ESI⁺] 414, 416 (M+H)⁺, (C₂₀H₂₀ClN₅OS)

### Synthesis of N-(4-(2-chlorophenyl)thiazol-2-yl)-5-(piperazine-1-carbonyl)picolinamide (Compound 451)

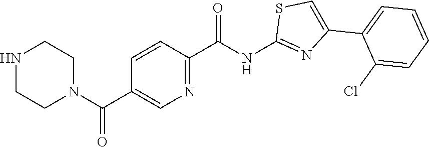 [SMILES: O=C(Nc1nc(-c2ccccc2Cl)cs1)c1ccc(C(=O)N2CCNCC2)cn1]

Compound N-(4-(2-chlorophenyl)thiazol-2-yl)-5-(piperazine-1-carbonyl)picolinamide was prepared from tert-butyl 4-(6-((4-(2-chlorophenyl)thiazol-2-yl)carbamoyl)nicotinoyl)piperazine-1-carboxylate (50 mg, 0.095 mmol), following a procedure similar to that described for the synthesis of 4-(2,4-dichlorophenyl)thiazol-2-amine, except that it was purified by Prep-HPLC with the following conditions: Column: XBridge Shield RP18 OBD Column, 30×150 mm, 5 μm; Mobile Phase A: water (plus 10 mmol/L NH₄HCO₃); Mobile Phase B: acetonitrile; Flow rate: 60 mL/min; Gradient: 55%-70% B in 8 min; Detector: UV 254/220 nm. The fractions containing the desired product were collected, concentrated under reduced pressure and lyophilized to afford N-(4-(2-chlorophenyl)thiazol-2-yl)-5-(piperazine-1-carbonyl)picolinamide as a yellow solid.

Yield 18 mg (44%). ¹H NMR (400 MHz, DMSO) δ 8.79 (d, J=2.0 Hz, 1H), 8.25 (d, J=8.0 Hz, 1H), 8.12 (dd, J=2.0, 8.0 Hz, 1H), 7.93 (dd, J=2.0, 7.6 Hz, 1H), 7.78 (s, 1H), 7.58 (dd, J=1.6, 8.0 Hz, 1H), 7.50-7.34 (m, 2H), 3.64-3.60 (m, 2H), 3.28-3.26 (m, 2H), 2.86-2.57 (m, 4H). Amide NH and aliphatic NH not observed. m/z: [ESI⁺] 428, 430 (M+H)⁺, (C₂₀H₁₈ClN₅O₂S)

### Synthesis of N-(4-(2-((dimethylamino)methyl)phenyl)thiazol-2-yl)-5-(4-(methylsulfonyl)piperazin-1-yl)picolinamide hemiformate (Compound 460)

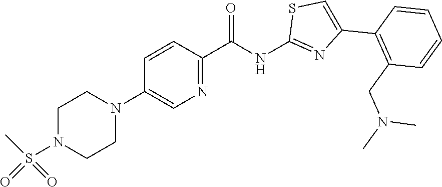 [SMILES: CN(C)Cc1ccccc1-c1csc(NC(=O)c2ccc(N3CCN(S(C)(=O)=O)CC3)cn2)n1]

Compound N-(4-(2-((dimethylamino)methyl)phenyl)thiazol-2-yl)-5-(4-(methylsulfonyl)piperazin-1-yl)picolinamide hemiformate was prepared from N-(4-bromothiazol-2-yl)-5-(4-(methylsulfonyl)piperazin-1-yl)picolinamide (60 mg, 0.13 mmol) and (2-((dimethylamino)methyl)phenyl)boronic acid (36 mg, 0.20 mmol), following a procedure similar to that described for the synthesis of methyl 4-(3,6-dihydro-2H-thiopyran-4-yl)benzoate and was isolated as an off-white solid.

Yield 8 mg (12%). ¹H NMR (400 MHz, DMSO) δ 11.67 (br s, 1H), 8.47 (d, J=2.8 Hz, 1H), 8.25 (s, 0.1H, HCOOH), 8.05 (d, J=8.8 Hz, 1H), 7.73-7.65 (m, 1H), 7.60 (s, 1H), 7.56 (dd, J=2.8, 8.8 Hz, 1H), 7.50-7.43 (m, 1H), 7.41-7.32 (m, 2H), 3.62-3.55 (m, 6H), 3.32-3.24 (m, 4H), 2.95 (s, 3H), 2.19 (s, 6H). m/z: [ESI⁺]501 (M+H)⁺, (C₂₃H₂₈N₆O₃S₂).

### Synthesis of 5-(4-acetylpiperazin-1-yl)-N-(4-(2-(hydroxymethyl)pyridin-3-yl)thiazol-2-yl)picolinamide (Compound 472)

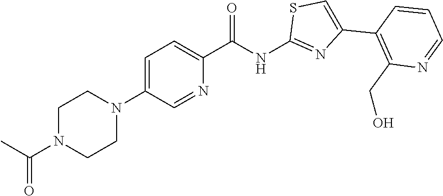 [SMILES: CC(=O)N1CCN(c2ccc(C(=O)Nc3nc(-c4cccnc4CO)cs3)nc2)CC1]

Compound 5-(4-acetylpiperazin-1-yl)-N-(4-(2-(hydroxymethyl)pyridin-3-yl)thiazol-2-yl)picolinamide was prepared from 5-(4-acetylpiperazin-1-yl)-N-(4-bromothiazol-2-yl)picolinamide (240 mg, 0.59 mmol) and (3-(4,4,5,5-tetramethyl-1,3,2-dioxaborolan-2-yl)pyridin-2-yl)methyl acetate (325 mg, 1.17 mmol), following a procedure similar to that described for the synthesis of methyl 4-(3,6-dihydro-2H-thiopyran-4-yl)benzoate, expect that bis(triphenylphosphine)palladium(II) chloride was used as the catalyst and was isolated as an off-white solid.

Yield 20 mg (8%). ¹H NMR (400 MHz, DMSO) δ 12.12 (br s, 1H), 8.55 (d, J=4.4 Hz, 1H), 8.44-8.40 (m, 1H), 8.13 (d, J=8.0 Hz, 1H), 8.03 (dd, J=1.2, 8.8 Hz, 1H), 7.68 (d, J=1.2 Hz, 1H), 7.50 (dd, J=2.8, 8.8 Hz, 1H), 7.46-7.40 (m, 1H), 5.34 (t, J=6.4 Hz, 1H), 4.69 (d, J=6.4 Hz, 2H), 3.67-3.58 (m, 4H), 3.54-3.47 (m, 2H), 3.46-3.40 (m, 2H), 2.06 (s, 3H). m/z: [ESI⁺] 439 (M+H)⁺, (C₂₁H₂₂N₆O₃S).

### Synthesis of 5-(4-acetylpiperazin-1-yl)-N-(4-(2-(methoxymethyl)phenyl)thiazol-2-yl)picolinamide (Compound 469)

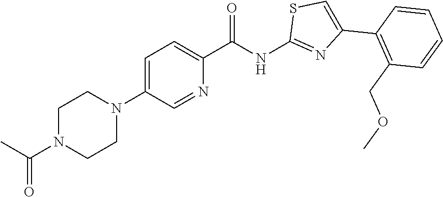 [SMILES: COCc1ccccc1-c1csc(NC(=O)c2ccc(N3CCN(C(C)=O)CC3)cn2)n1]

Compound 5-(4-acetylpiperazin-1-yl)-N-(4-(2-(methoxymethyl)phenyl)thiazol-2-yl)picolinamide was prepared from 5-(4-acetylpiperazin-1-yl)-N-(4-bromothiazol-2-yl)picolinamide (300 mg, 0.73 mmol) and (2-(methoxymethyl)phenyl)boronic acid (243 mg, 1.46 mmol), following a procedure similar to that described for the synthesis of methyl 4-(3,6-dihydro-2H-thiopyran-4-yl)benzoate, expect that bis(triphenylphosphine)palladium(II) chloride was used as the catalyst and was isolated as a yellow solid.

Yield 27 mg (8%). ¹H NMR (400 MHz, DMSO) δ 11.55 (br s, 1H), 8.41 (d, J=2.8 Hz, 1H), 8.02 (d, J=8.8 Hz, 1H), 7.73-7.66 (m, 11H), 7.54-7.45 (m, 2H), 7.42-7.34 (m, 3H), 4.61 (s, 2H), 3.61 (t, J=5.2 Hz, 4H), 3.52-3.46 (m, 2H), 3.45-3.40 (m, 2H), 3.32 (s, 3H), 2.06 (s, 3H). m/z: [ESI⁺] 452 (M+H)⁺, (C₂₃H₂₅N₅O₃S).

### Synthesis of 5-(4-acetylpiperazin-1-yl)-N-(4-(2-(hydroxymethyl)phenyl)thiazol-2-yl)picolinamide (Compound 470)

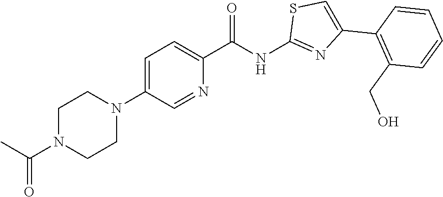 [SMILES: CC(=O)N1CCN(c2ccc(C(=O)Nc3nc(-c4ccccc4CO)cs3)nc2)CC1]

Compound 5-(4-acetylpiperazin-1-yl)-N-(4-(2-(hydroxymethyl)phenyl)thiazol-2-yl)picolinamide was prepared from 5-(4-acetylpiperazin-1-yl)-N-(4-bromothiazol-2-yl)picolinamide (300 mg, 0.73 mmol) and (2-(hydroxymethyl)phenyl)boronic acid (223 mg, 1.47 mmol), following a procedure similar to that described for the synthesis of methyl 4-(3,6-dihydro-2H-thiopyran-4-yl)benzoate, expect that bis(triphenylphosphine)palladium(II) chloride was used as the catalyst and was isolated as a yellow solid.

Yield 165 mg (52%). ¹H NMR (400 MHz, DMSO) δ 11.97 (br s, 1H), 8.40 (d, J=2.8 Hz, 1H), 8.01 (d, J=8.8 Hz, 1H), 7.66 (dd, J=2.0, 6.8 Hz, 1H), 7.54 (dd, J=2.0, 6.8 Hz, 1H), 7.50-7.43 (m, 2H), 7.40-7.30 (m, 2H), 5.32 (t, J=6.0 Hz, 1H), 4.60 (d, J=6.0 Hz, 2H), 3.68-3.55 (m, 4H), 3.52-3.44 (m, 2H), 3.40-3.35 (m, 2H), 2.05 (s, 3H). m/z: [ESI⁺] 438 (M+H)⁺, (C₂₂H₂₃N₅O₃S).

### Synthesis of 5-(4-acetylpiperazin-1-yl)-N-(4-(2-(methoxymethyl)pyridin-3-yl)thiazol-2-yl)picolinamide (Compound 471) (Method 1)

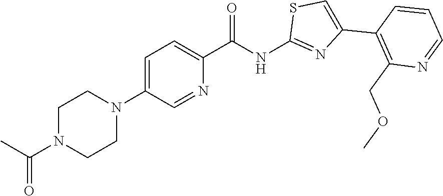 [SMILES: COCc1ncccc1-c1csc(NC(=O)c2ccc(N3CCN(C(C)=O)CC3)cn2)n1]

Compound 5-(4-acetylpiperazin-1-yl)-N-(4-(2-(methoxymethyl)pyridin-3-yl)thiazol-2-yl)picolinamide was prepared from 5-(4-acetylpiperazin-1-yl)-N-(4-bromothiazol-2-yl)picolinamide (366 mg, 0.89 mmol) and 2-(methoxymethyl)-3-(4,4,5,5-tetramethyl-1,3,2-dioxaborolan-2-yl)pyridine (300 mg, 1.20 mmol), following a procedure similar to that described for the synthesis of methyl 4-(3,6-dihydro-2H-thiopyran-4-yl)benzoate, expect that bis(triphenylphosphine)palladium(II) chloride was used as the catalyst and was isolated as an off-white solid.

Yield 23 mg (6%). ¹H NMR (400 MHz, DMSO) δ 11.69 (br s, 1H), 8.56 (dd, J=1.6, 4.8 Hz, 1H), 8.43 (d, J=2.8 Hz, 1H), 8.15 (dd, J=1.6, 7.6 Hz, 1H), 8.03 (d, J=8.8 Hz, 1H), 7.56 (s, 1H), 7.53-7.43 (m, 2H), 4.68 (s, 2H), 3.66-3.58 (m, 4H), 3.55-3.49 (m, 2H), 3.47-3.41 (m, 2H), 3.31 (s, 3H), 2.06 (s, 3H). m/z: [ESI⁺]453 (M+H)⁺, (C₂₂H₂₄N₆O₃S).

### Synthesis of 5-(4-acetylpiperazin-1-yl)-N-(4-(2-(methoxymethyl)pyridin-3-yl)thiazol-2-yl)picolinamide (Compound 471) (Method 2)

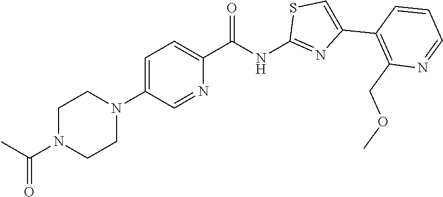 [SMILES: COCc1ncccc1-c1csc(NC(=O)c2ccc(N3CCN(C(C)=O)CC3)cn2)n1]

Compound 5-(4-acetylpiperazin-1-yl)-N-(4-(2-(methoxymethyl)pyridin-3-yl)thiazol-2-yl)picolinamide was prepared from 5-(4-acetylpiperazin-1-yl)-N-(4-bromothiazol-2-yl)picolinamide (20.00 g, 48.75 mmol) and 2-(methoxymethyl)-3-(4,4,5,5-tetramethyl-1,3,2-dioxaborolan-2-yl)pyridine (14.57 g, 58.49 mmol), following a procedure similar to that described for the synthesis of methyl 4-(3,6-dihydro-2H-thiopyran-4-yl)benzoate and was isolated as a dark yellow solid.

Yield 5.10 g (23%). ¹H NMR (400 MHz, DMSO) δ 11.69 (br s, 1H), 8.56 (dd, J=1.6, 4.8 Hz, 1H), 8.43 (d, J=2.8 Hz, 1H), 8.15 (dd, J=1.6, 7.6 Hz, 1H), 8.03 (d, J=8.8 Hz, 1H), 7.56 (s, 1H), 7.53-7.43 (m, 2H), 4.68 (s, 2H), 3.66-3.58 (m, 4H), 3.55-3.49 (m, 2H), 3.47-3.41 (m, 2H), 3.31 (s, 3H), 2.06 (s, 3H). m/z: [ESI⁺]453 (M+H)⁺, (C₂₂H₂₄N₆O₃S).

### Synthesis of N-(4-(2-chlorophenyl)thiazol-2-yl)-5-(4-(3-hydroxypropanoyl)piperazin-1-yl)picolinamide (Compound 455)

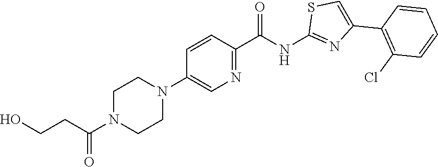 [SMILES: O=C(Nc1nc(-c2ccccc2Cl)cs1)c1ccc(N2CCN(C(=O)CCO)CC2)cn1]

To a stirred solution of 5-(4-(3-((tert-butyldiphenylsilyl)oxy)propanoyl)piperazin-1-yl)-N-(4-(2-chlorophenyl)thiazol-2-yl)picolinamide (500 mg, 0.70 mmol) in THF (8 mL) and water (2 mL) was added tetrabutylammonium fluoride trihydrate (TBAF) (900 mg, 2.85 mmol) at room temperature. The resulting solution was stirred overnight at 50° C., under a nitrogen atmosphere. The mixture was cooled to room temperature and concentrated under reduced pressure. The residue was purified by reverse phase flash chromatography with the following conditions: Column: WelFlash TM C18-I, 20-40 μm, 120 g; Eluent A: water (plus 10 mmol/L NH₄HCO₃); Eluent B: acetonitrile; Gradient: 27%-47% B in 25 min; How rate: 60 mL/min; Detector: UV 220/254 nm. The fractions containing the desired product were collected, concentrated under reduced pressure and lyophilized to afford N-(4-(2-chlorophenyl)thiazol-2-yl)-5-(4-(3-hydroxypropanoyl)piperazin-1-yl)picolinamide as an off-white solid.

Yield 31 mg (9%). ¹H NMR (400 MHz, DMSO) δ 11.58 (br s, 1H), 8.37 (s, 1H), 8.00 (d, J=8.8 Hz, 1H), 7.92 (dd, J=1.6, 7.6 Hz, 1H), 7.69 (s, 1H), 7.54 (d, J=7.6 Hz, 1H), 7.47-7.31 (m, 3H), 4.58 (br s, 1H), 3.73-3.58 (m, 6H), 3.52-3.41 (m, 4H), 2.54-2.52 (m, 2H). m/z: [ESI⁺] 472, 474 (M+H)⁺, (C₂₂H₂₂ClN₅O₃S).

### Synthesis of 5-(4-hydroxypiperidin-1-yl)-N-(4-(2-(methoxymethyl)phenyl)thiazol-2-yl)picolinamide (Compound 468)

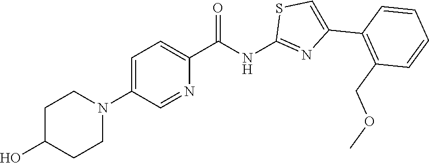 [SMILES: COCc1ccccc1-c1csc(NC(=O)c2ccc(N3CCC(O)CC3)cn2)n1]

Compound 5-(4-hydroxypiperidin-1-yl)-N-(4-(2-(methoxymethyl)phenyl)thiazol-2-yl)picolinamide was prepared from 5-(4-((tert-butyldimethylsilyl)oxy)piperidin-1-yl)-N-(4-(2-(methoxymethyl)phenyl)thiazol-2-yl)picolinamide (200 mg, 0.37 mmol), following a procedure similar to that described for the synthesis of N-(4-(2-chlorophenyl)thiazol-2-yl)-5-(4-(3-hydroxypropanoyl)piperazin-1-yl)picolinamide and was isolated as a light yellow solid.

Yield 60 mg (38%). ¹H NMR (400 MHz, DMSO) δ 11.55 (br s, 1H), 8.42 (d, J=2.8 Hz, 1H), 7.99 (d, J=8.8 Hz, 1H), 7.76-7.64 (m, 1H), 7.55-7.45 (m, 2H), 7.43-7.35 (m, 3H), 4.78 (d, J=4.4 Hz, 1H), 4.62 (s, 2H), 3.87-3.70 (m, 3H), 3.31 (s, 3H), 3.23-3.11 (m, 2H), 1.90-1.79 (m, 2H), 1.52-1.40 (m, 2H). m/z: [ESI⁺] 425 (M+H)⁺, (C₂₂H₂₄N₄O₃S).

### Synthesis of (1r,4r)-N-(4-(2-chlorophenyl)thiazol-2-yl)-4-(4-(methylsulfonyl)piperazin-1-yl)cyclohexane-1-carboxamide (Compound 435) Synthesis of (1s,4s)-N-(4-(2-chlorophenyl)thiazol-2-yl)-4-(4-(methylsulfonyl)piperazin-1-yl)cyclohexane-1-carboxamide (Compound 434)

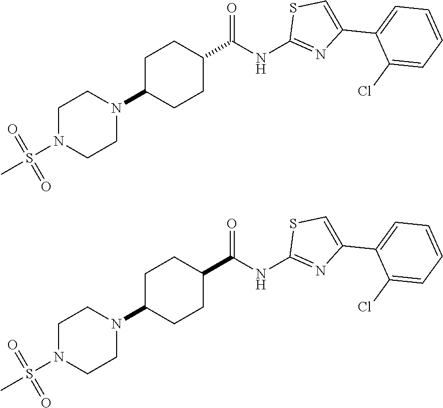

Compound (1r,4r)-N-(4-(2-chlorophenyl)thiazol-2-yl)-4-(4-(methylsulfonyl)piperazin-1-yl)cyclohexane-1-carboxamide and (1s,4s)-N-(4-(2-chlorophenyl)thiazol-2-yl)-4-(4-(methylsulfonyl)piperazin-1-yl)cyclohexane-1-carboxamide were prepared from N-(4-(2-chlorophenyl)thiazol-2-yl)-4-oxocyclohexane-1-carboxamide (300 mg, 0.90 mmol) and 1-(methylsulfonyl)piperazine (294 mg, 1.79 mmol), following a procedure similar to that described for the synthesis of benzyl 4-(3-(methoxycarbonyl)bicyclo[1.1.1]pentan-1-yl)piperazine-1-carboxylate. The two isomers were separated by chiral-HPLC with the following conditions: Column: CHIRALPAK IC-3, 4.6×50 mm, 3.0 μm; Mobile phase: Hexane (plus 0.2% isopropylamine, v/v):EtOH=70:30; Flow rate: 1.0 m/min. The faster eluting peak was concentrated to afford (1r,4r)-N-(4-(2-chlorophenyl)thiazol-2-yl)-4-(4-(methylsulfonyl)piperazin-1-yl)cyclohexane-1-carboxamide as an off-white solid (trans or cis not determined).

Yield 15.8 mg (4%). ¹H NMR (400 MHz, DMSO) δ 12.20 (br s, 1H), 7.83 (dd, J=2.0, 7.6 Hz, 1H), 7.60-7.53 (m, 2H), 7.46-7.34 (m, 2H), 3.11 (t, J=4.8 Hz, 4H), 2.87 (s, 3H), 2.73-2.65 (m, 111), 2.57-2.52 (m, 4H), 2.31-2.23 (m, 1H), 1.99-1.88 (m, 2H), 1.88-1.78 (m, 2H), 1.61-1.45 (m, 4H). m/z: [ESI⁺] 483, 485 (M+H)⁺, (C₂₁H₂₇ClN₄O₃S₂).

The slower eluting peak was concentrated to afford (1s,4s)-N-(4-(2-chlorophenyl)thiazol-2-yl)-4-(4-(methylsulfonyl)piperazin-1-yl)cyclohexane-1-carboxamide as an off-white solid (trans or cis not determined).

Yield 9.2 mg (2%). ¹H NMR (400 MHz, DMSO) δ 12.25 (br s, 1H), 7.83 (dd, J=2.0, 7.6 Hz, 11), 7.60-7.52 (m, 2H), 7.47-7.34 (m, 2H), 3.08 (t, J=4.8 Hz, 4H), 2.87 (s, 3H), 2.63-2.56 (m, 4H), 2.49-2.30 (m, 21), 2.00-1.79 (m, 4H), 1.56-1.40 (m, 2H), 1.33-1.19 (m, 2H). m/z: [ESI⁺] 483, 485 (M+H)⁺, (C₂₁H₂₇ClN₄O₃S₂).

### Synthesis of (1s,3s)-N-(4-(2-chlorophenyl)thiazol-2-yl)-3-morpholinocyclobutane-1-carboxamide (Compound 421)

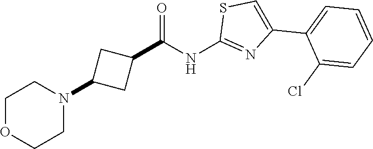

Compound (1s,3s)-N-(4-(2-chlorophenyl)thiazol-2-yl)-3-morpholinocyclobutane-1-carboxamide was prepared from N-(4-(2-chlorophenyl)thiazol-2-yl)-3-oxocyclobutane-1-carboxamide (153 mg, 0.50 mmol) and morpholine (87 mg, 1.00 mmol), following a procedure similar to that described for the synthesis of benzyl 4-(3-(methoxycarbonyl)bicyclo[1.1.1]pentan-1-yl)piperazine-1-carboxylate and was isolated as an off-white solid.

Yield 74 mg (39%). ¹H NMR (400 MHz, DMSO) δ 12.28 (br s, 1H), 7.83 (dd, J=2.0, 7.6 Hz, 1H), 7.59 (s, 1H), 7.55 (d, J=7.6 Hz, 1H), 7.46-7.34 (m, 2H), 3.64-3.50 (m, 4H), 3.09-2.97 (m, 1H), 2.76-2.66 (m, 1H), 2.31-2.20 (m, 6H), 2.11-1.99 (m, 2H). m/z: [ESI⁺] 378, 380 (M+H)⁺, (C₁₈H₂₀ClN₃O₂S).

### Synthesis of N-(4-(2-chlorophenyl)thiazol-2-yl)-5-(2-methyl-2,7-diazaspiro[3.5]nonan-7-yl)picolinamide (Compound 456)

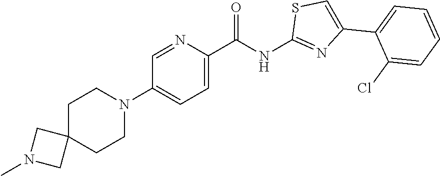 [SMILES: CN1CC2(CCN(c3ccc(C(=O)Nc4nc(-c5ccccc5Cl)cs4)nc3)CC2)C1]

Compound N-(4-(2-chlorophenyl)thiazol-2-yl)-5-(2-methyl-2,7-diazaspiro[3.5]nonan-7-yl)picolinamide was prepared from N-(4-(2-chlorophenyl)thiazol-2-yl)-5-(2,7-diazaspiro[3.5]nonan-7-yl)picolinamide hydrochloride (400 mg, 0.84 mmol) and formaldehyde (164 mg, 5.46 mmol), following a procedure similar to that described for the synthesis of benzyl 4-(3-(methoxycarbonyl)bicyclo[1.1.1]pentan-1-yl)piperazine-1-carboxylate and was isolated as a off-white solid.

Yield 38 mg (10%). ¹H NMR (400 MHz, DMSO) δ 9.99 (br s, 1H), 8.43 (d, J=2.8 Hz, 1H), 8.06 (d, J=8.8 Hz, 1H), 7.91 (dd, J=2.0, 7.6, Hz, 1H), 7.70 (s, 1H), 7.61-7.53 (m, 2H), 7.48-7.35 (m, 2H), 4.08-3.99 (m, 2H), 3.87-3.77 (m, 2H), 3.55-3.48 (m, 2H), 3.45-3.38 (m, 2H), 2.88 (s, 3H), 1.94-1.86 (m, 4H). m/z: [ESI⁺]454, 456 (M+H)⁺, (C₂₃H₂₄ClN₅OS).

### Synthesis of N-(4-(2-(hydroxymethyl)phenyl)thiazol-2-yl)-5-(4-(methylsulfonyl)piperazin-1-yl)picolinamide (Compound 454)

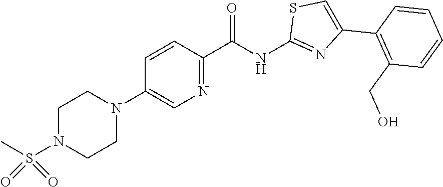 [SMILES: CS(=O)(=O)N1CCN(c2ccc(C(=O)Nc3nc(-c4ccccc4CO)cs3)nc2)CC1]

To a stirred solution of N-(4-(2-formylphenyl)thiazol-2-yl)-5-(4-(methylsulfonyl)piperazin-1-yl)picolinamide (50 mg, 0.11 mmol) in methanol (2 mL) was added sodium borohydride (8 mg, 0.21 mmol), at room temperature and was stirred for 30 min. The resulting mixture was concentrated under reduced pressure. The residue was purified by reverse phase flash chromatography with the following conditions: Column: WelFlash TM C18-I, 20-40 μm, 80 g; Eluent A: water (plus 10 mmol/L NH₄HCO₃); Eluent B: acetonitrile; Gradient: 43%-63% B in 25 min; Flow rate: 60 mb/min; Detector: UV 220/254 nm. Desired fractions were collected, concentrated under reduced pressure and lyophilized to afford N-(4-(2-(hydroxymethyl)phenyl)thiazol-2-yl)-5-(4-(methylsulfonyl)piperazin-1-yl)picolinamide as an off-white solid.

Yield 11 mg (22%). ¹H NMR (400 MHz, DMSO) δ 11.99 (br s, 1H), 8.46 (d, J=2.8 Hz, 1H), 8.04 (d, J=8.8 Hz, 1H), 7.66 (dd, J=2.0, 7.2 Hz, 1H), 7.58-7.52 (m, 2H), 7.47 (s, 1H), 7.42-7.32 (m, 2H), 5.29 (t, J=6.4 Hz, 1H), 4.59 (d, J=6.4 Hz, 2H), 3.58 (dd, J=3.6, 6.4 Hz, 4H), 3.29 (dd, J=3.6, 6.4 Hz, 4H), 2.94 (s, 3H). m/z: [ESI⁺] 474 (M+H)⁺, (C₂₁H₂₃N₅O₄S₂).

### Synthesis of 4-(6-((4-(2-chlorophenyl)thiazol-2-yl)carbamoyl)pyridin-3-yl)-N-methylpiperazine-1-carboxamide (Compound 453)

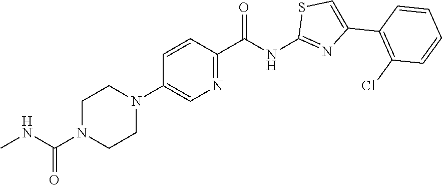 [SMILES: CNC(=O)N1CCN(c2ccc(C(=O)Nc3nc(-c4ccccc4Cl)cs3)nc2)CC1]

To a stirred solution of N-(4-(2-chlorophenyl)thiazol-2-yl)-5-(piperazin-1-yl)picolinamide hydrochloride (200 mg, 0.46 mmol) in DCM (4 mL), were added N-methylcarbamoyl chloride (93 mg, 1.00 mmol) and triethylamine (203 mg, 2.01 mmol), at room temperature under a nitrogen atmosphere. The reaction was stirred overnight at room temperature. The resulting mixture was concentrated under reduced pressure. The residue was purified by reverse phase flash chromatography with the following conditions: Column: WelFlash TM C18-L, 20-40 μm, 120 g; Eluent A: water (plus 10 mmol/L NH₄HCO₃); Eluent B: acetonitrile; Gradient: 49%-69% B in 25 min; How rate: 60 mL/min; Detector: UV 220/254 nm. The fractions containing the desired product were collected and concentrated under reduced pressure to afford 4-(6-((4-(2-chlorophenyl)thiazol-2-yl)carbamoyl)pyridin-3-yl)-N-methylpiperazine-1-carboxamide as an off-white solid.

Yield 39 mg (19%). ¹H NMR (400 MHz, DMSO) δ 11.60 (br s, 1H), 8.42 (d, J=2.8 Hz, 1H), 8.01 (d, J=8.8 Hz, 1H), 7.92 (dd, J=2.0, 7.6 Hz, 1H), 7.70 (s, 1H), 7.56 (dd, J=1.6, 7.6 Hz, 1H), 7.52-7.34 (m, 3H), 6.56 (q, J=4.4 Hz, 1H), 3.47 (dd, J=3.6, 7.2 Hz, 4H), 3.44-3.40 (m, 4H), 2.61 (d, J=4.4 Hz, 3H). m/z: [ESI⁺] 457, 459 (M+H)⁺, (C₂₁H₂₁ClN₆O₂S).

### Synthesis of N-(4-(2-chlorophenyl)thiazol-2-yl)-5-(4-(methylsulfonyl)piperazin-1-yl)picolinamide (Compound 403)

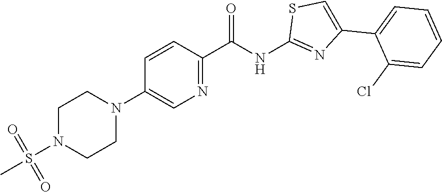 [SMILES: CS(=O)(=O)N1CCN(c2ccc(C(=O)Nc3nc(-c4ccccc4Cl)cs3)nc2)CC1]

Compound N-(4-(2-chlorophenyl)thiazol-2-yl)-5-(4-(methylsulfonyl)piperazin-1-yl)picolinamide was prepared from N-(4-(2-chlorophenyl)thiazol-2-yl)-5-(piperazin-1-yl)picolinamide hydrochloride (50 mg, 0.12 mmol) and methanesulfonyl chloride (36 mg, 0.31 mmol), following a procedure similar to that described for the synthesis of methyl 3-(4-(methylsulfonyl)piperazin-1-yl)bicyclo[1.1.1]pentane-1-carboxylate and was isolated as an off-white solid.

Yield 15 mg (27%). ¹H NMR (400 MHz, DMSO) δ 11.68 (br s, 1H), 8.47 (d, J=2.8 Hz, 1H), 8.04 (d, J=8.8 Hz, 1H), 7.93 (dd, J=2.0, 7.6 Hz, 1H), 7.72 (s, 1H), 7.59-7.53 (m, 2H), 7.48-7.37 (m, 2H), 3.62-3.56 (m, 4H), 3.30-3.24 (m, 4H), 2.95 (s, 3H). m/z: [ESI⁺] 478, 480 (M+H)⁺, (C₂₀H₂₀ClN₅O₃S₂).

### Synthesis of N-(4-(2-chlorophenyl)thiazol-2-yl)-5-((1-(methylsulfonyl)piperidin-4-yl)oxy)picolinamide (Compound 438)

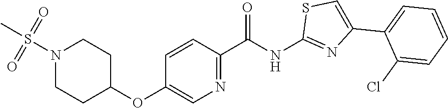 [SMILES: CS(=O)(=O)N1CCC(Oc2ccc(C(=O)Nc3nc(-c4ccccc4Cl)cs3)nc2)CC1]

Compound N-(4-(2-chlorophenyl)thiazol-2-yl)-5-((1-(methylsulfonyl)piperidin-4-yl)oxy)picolinamide was prepared from N-(4-(2-chlorophenyl)thiazol-2-yl)-5-(piperidin-4-yloxy)picolinamide hydrochloride (55 mg, 0.12 mmol) and methanesulfonyl chloride (28 mg, 0.24 mmol), following a procedure similar to that described for the synthesis of methyl 3-(4-(methylsulfonyl)piperazin-1-yl)bicyclo[1.1.1]pentane-1-carboxylate and was isolated as an off-white solid.

Yield 15 mg (25%). ¹H NMR (400 MHz, DMSO) δ 11.92 (br s, 1H), 8.47 (d, J=2.8 Hz, 1H), 8.18 (d, J=8.8 Hz, 1H), 7.92 (dd, J=2.0, 7.6 Hz, 1H), 7.78-7.70 (m, 2H), 7.57 (dd, J=1.6, 7.6 Hz, 1H), 7.51-7.34 (m, 2H), 4.90-4.81 (m, 1H), 3.35-3.29 (m, 2H), 3.24-3.10 (m, 2H), 2.93 (s, 3H), 2.20-1.99 (m, 2H), 1.88-1.72 (m, 2H). m/z: [ESI⁺] 493, 495 (M+H)⁺, (C₂₁H₂₁ClN₄O₄S₂).

### Synthesis of 5-((1-acetylpiperidin-4-yl)oxy)-N-(4-(2-chlorophenyl)thiazol-2-yl)picolinamide (Compound 440)

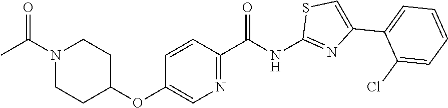 [SMILES: CC(=O)N1CCC(Oc2ccc(C(=O)Nc3nc(-c4ccccc4Cl)cs3)nc2)CC1]

To a stirred mixture of N-(4-(2-chlorophenyl)thiazol-2-yl)-5-(piperidin-4-yloxy)picolinamide hydrochloride (55 mg, 0.12 mmol) and triethylamine (37 mg, 0.37 mmol) in DCM (2 mL) was added acetic anhydride (25 mg, 0.25 mmol), dropwise at 0° C. under a nitrogen atmosphere. The reaction was stirred for 4 h at room temperature. The resulting solution was concentrated under reduced pressure. The residue was purified by reverse phase flash chromatography with the following conditions Column: WelFlash TM C18-I, 20-40 μm, 120 g; Eluent A: water (plus 10 mmol/L NH₄HCO₃); Eluent B: acetonitrile; Gradient: 45%-65% B in 20 min; Flow rate: 60 mL/min; Detector: UV 220/254 nm. The fractions containing the desired product were collected and concentrated under reduced pressure to afford 5-((1-acetylpiperidin-4-yl)oxy)-N-(4-(2-chlorophenyl)thiazol-2-yl)picolinamide as an off-white solid.

Yield 15 mg (27%). ¹H NMR (400 MHz, DMSO) δ 11.90 (br s, 1H), 8.46 (d, J=2.8 Hz, 1H), 8.17 (d, J=8.8 Hz, 1H), 7.92 (dd, J=2.0, 7.6 Hz, 1H), 7.78-7.72 (m, 2H), 7.57 (dd, J=1.6, 7.6 Hz, 1H), 7.49-7.33 (m, 2H), 4.97-4.78 (m, 1H), 3.95-3.82 (m, 1H), 3.77-3.64 (m, 1H), 3.38-3.20 (m, 2H), 2.10-2.02 (m, 4H), 2.00-1.90 (m, 1H), 1.76-1.62 (m, 1H), 1.62-1.50 (m, 1H). m/z: [ESI⁺] 457, 459 (M+H)⁺, (C₂H₂₁ClN₄O₃S).

### Synthesis of 5-(6-acetyl-2,6-diazaspiro[3.3]heptan-2-yl)-N-(4-(2-chlorophenyl)thiazol-2-yl)picolinamide (Compound 449)

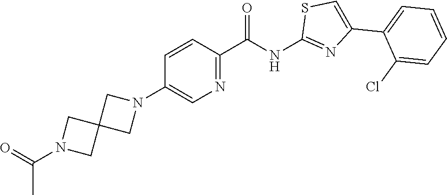 [SMILES: CC(=O)N1CC2(C1)CN(c1ccc(C(=O)Nc3nc(-c4ccccc4Cl)cs3)nc1)C2]

Compound 5-(6-acetyl-2,6-diazaspiro[3.3]heptan-2-yl)-N-(4-(2-chlorophenyl)thiazol-2-yl)picolinamide was prepared from N-(4-(2-chlorophenyl)thiazol-2-yl)-5-(2,6-diazaspiro[3.3]heptan-2-yl)picolinamide 2,2,2-trifluoroacetate salt (64 mg, 0.12 mmol) and acetic anhydride (25 mg, 0.25 mmol), following a procedure similar to that described for the synthesis of 5-((1-acetylpiperidin-4-yl)oxy)-N-(4-(2-chlorophenyl)thiazol-2-yl)picolinamide and was isolated as an off-white solid.

Yield 20 mg (36%). ¹H NMR (400 MHz, DMSO) δ 11.54 (br s, 1H), 7.99 (d, J=8.8 Hz, 1H), 7.95-7.88 (m, 2H), 7.70 (s, 1H), 7.57 (dd, J=1.6, 7.6 Hz, 1H), 7.48-7.33 (m, 2H), 6.98 (dd, J=2.8, 8.8 Hz, 1H), 4.33 (s, 2H), 4.21 (s, 4H), 4.05 (s, 2H), 1.76 (s, 3H). m/z: [ESI⁺] 454, 456 (M+H)⁺, (C₂₂H₂₀ClN₅O₂S).

### Synthesis of 5-(4-acetylpiperazin-1-yl)-N-(4-(2-chlorophenyl)thiazol-2-yl)picolinamide (Compound 442)

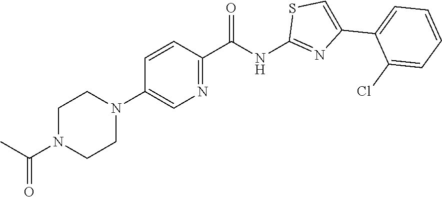 [SMILES: CC(=O)N1CCN(c2ccc(C(=O)Nc3nc(-c4ccccc4Cl)cs3)nc2)CC1]

Compound 5-(4-acetylpiperazin-1-yl)-N-(4-(2-chlorophenyl)thiazol-2-yl)picolinamide was prepared from N-(4-(2-chlorophenyl)thiazol-2-yl)-5-(piperazin-1-yl)picolinamide hydrochloride (109 mg, 0.25 mmol) and acetic anhydride (51 mg, 0.50 mmol), following a procedure similar to that described for the synthesis of 5-((1-acetylpiperidin-4-yl)oxy)-N-(4-(2-chlorophenyl)thiazol-2-yl)picolinamide and was isolated as an off-white solid.

Yield 33 mg (30%). ¹H NMR (400 MHz, DMSO) δ 11.62 (br s, 1H), 8.41 (d, J=2.8 Hz, 1H), 8.02 (d, J=8.8 Hz, 1H), 7.93 (dd, J=1.6, 7.6 Hz, 1H), 7.71 (s, 1H), 7.56 (dd, J=1.6, 7.6 Hz, 1H), 7.52-7.31 (m, 3H), 3.72-3.56 (m, 4H), 3.54-3.46 (m, 2H), 3.46-3.39 (m, 2H), 2.06 (s, 3H). m/z: [ESI⁺] 442, 444 (M+H)⁺, (C₂₁H₂₀ClN₅O₂S).

### Synthesis of 5-morpholino-N-(4-(tetrahydro-2H-pyran-4-yl)thiazol-2-yl)picolinamide (Compound 393)

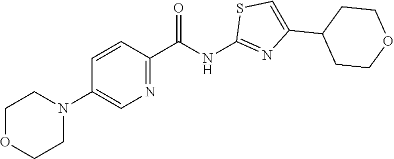 [SMILES: O=C(Nc1nc(C2CCOCC2)cs1)c1ccc(N2CCOCC2)cn1]

Compound 5-morpholino-N-(4-(tetrahydro-2H-pyran-4-yl)thiazol-2-yl)picolinamide was prepared from 5-morpholinopicolinic acid (316 mg, 1.52 mmol) and 4-(tetrahydro-2H-pyran-4-yl)thiazol-2-amine (280 mg, 1.52 mmol), following a procedure similar to that described for the synthesis of methyl (1s,3s)-3-((4-(2-chlorophenyl)thiazol-2-yl)carbamoyl)cyclobutane-1-carboxylate and was isolated as a light yellow solid.

Yield 136 mg (24%). ¹H NMR (400 MHz, DMSO) δ 11.39 (br s, 1H), 8.40 (d, J=2.8 Hz, 1H), 7.99 (d, J=8.8 Hz, 1H), 7.48 (dd, J=2.8, 8.8 Hz, 1H), 6.87 (s, 1H), 3.98-3.87 (m, 2H), 3.79-3.69 (m, 4H), 3.48-3.35 (m, 6H), 2.93-2.75 (m, 1H), 1.92-1.79 (m, 2H), 1.73-1.56 (m, 2H). m/z: [ESI⁺] 375 (M+H)⁺, (C₁₈H₂₂N₄O₃S).

### Synthesis of N-(4-(2-chlorophenyl)thiazol-2-yl)-4-(morpholine-4-carbonyl)benzamide (Compound 384)

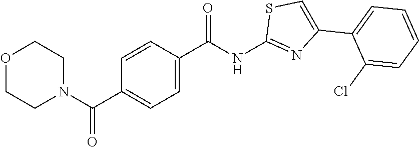 [SMILES: O=C(Nc1nc(-c2ccccc2Cl)cs1)c1ccc(C(=O)N2CCOCC2)cc1]

Compound N-(4-(2-chlorophenyl)thiazol-2-yl)-4-(morpholine-4-carbonyl)benzamide was prepared from 4-((4-(2-chlorophenyl)thiazol-2-yl)carbamoyl)benzoic acid (50 mg, 0.14 mmol) and morpholine (13 mg, 0.15 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 20 mg (34%). ¹H NMR (400 MHz, DMSO) δ 12.93 (br s, 1H), 8.19 (d, J=8.4 Hz, 2H), 7.91 (dd, J=2.0, 7.6 Hz, 1H), 7.70 (s, 1H), 7.59 (d, J=8.4 Hz, 2H, 2H), 7.50-7.36 (m, 3H), 3.65-3.30 (m, 8H). m/z: [ESI⁺] 428, 430 (M+H)⁺, (C₂₁H₁₈ClN₃O₃S).

### Synthesis of N-(4-(2-chlorophenyl)thiazol-2-yl)-4-(1,1-dioxidothiomorpholine-4-carbonyl)benzamide (Compound 385)

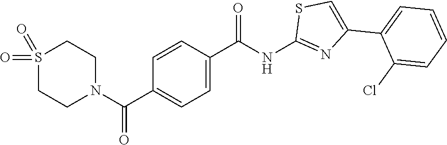 [SMILES: O=C(Nc1nc(-c2ccccc2Cl)cs1)c1ccc(C(=O)N2CCS(=O)(=O)CC2)cc1]

Compound N-(4-(2-chlorophenyl)thiazol-2-yl)-4-(1,1-dioxidothiomorpholine-4-carbonyl)benzamide was prepared from 4-((4-(2-chlorophenyl)thiazol-2-yl)carbamoyl)benzoic acid (50 mg, 0.15 mmol) and thiomorpholine 1,1-dioxide (21 mg, 0.16 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 8.4 mg (13%). ¹H NMR (400 MHz, DMSO) δ 12.93 (br s, 1H), 8.20 (d, J=8.4 Hz, 2H), 7.91 (dd, J=2.0, 7.6 Hz, 1H), 7.70 (s, 1H), 7.68 (d, J=8.4 Hz, 2H), 7.58 (dd, J=1.6, 7.6 Hz, 1H), 7.51-7.34 (m, 211), 4.16-3.93 (m, 2H), 3.78-3.60 (m, 2H), 3.35-3.30 (m, 4H). m/z: [ESI⁺] 476,478 (M+H)⁺, (C₂₁H₁₈ClN₃O₄S₂).

### Synthesis of N′-(4-(2-chlorophenyl)thiazol-2-yl)-N,N-dimethylterephthalamide (Compound 389)

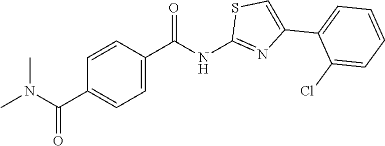 [SMILES: CN(C)C(=O)c1ccc(C(=O)Nc2nc(-c3ccccc3Cl)cs2)cc1]

Compound N′-(4-(2-chlorophenyl)thiazol-2-yl)-N⁴,N⁴-dimethylterephthalamide was prepared from 4-((4-(2-chlorophenyl)thiazol-2-yl)carbamoyl)benzoic acid (50 mg, 0.14 mmol) and dimethylamine hydrochloride (13 mg, 0.16 mmol), following a procedure similar to that described for the synthesis of methyl (1s,3s)-3-((4-(2-chlorophenyl)thiazol-2-yl)carbamoyl)cyclobutane-1-carboxylate and was isolated as a yellow solid.

Yield 14 mg (26%). ¹H NMR (400 MHz, DMSO) δ 12.30 (br s, 1H), 8.17 (d, J=8.4 Hz, 2H), 7.91 (dd, J=2.0, 7.6 Hz, 1H), 7.69 (s, 1H), 7.61-7.53 (m, 3H), 7.50-7.35 (m, 2H), 3.02 (s, 3H), 2.92 (s, 3H). m/z: [ESI⁺] 386, 388 (M+H)⁺, (C₁₉H₁₆ClN₃O₂S).

### Synthesis of N-(4-(2-chlorophenyl)thiazol-2-yl)-5-((1-methylpiperidin-4-yl)oxy)picolinamide (Compound 439)

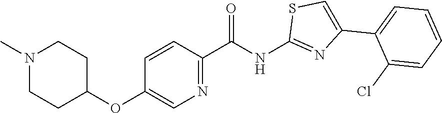 [SMILES: CN1CCC(Oc2ccc(C(=O)Nc3nc(-c4ccccc4Cl)cs3)nc2)CC1]

Compound N-(4-(2-chlorophenyl)thiazol-2-yl)-5-((1-methylpiperidin-4-yl)oxy)picolinamide was prepared from 5-((1-methylpiperidin-4-yl)oxy)picolinic acid (400 mg, 1.69 mmol) and 4-(2-chlorophenyl)thiazol-2-amine (357 mg, 1.70 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 30 mg (4%). ¹H NMR (400 MHz, DMSO) δ 11.87 (br s, 1H), 8.42 (d, J=2.8 Hz, 1H), 8.15 (d, J=8.8 Hz, 1H), 7.92 (dd, J=2.0, 7.6, Hz, 1H), 7.74 (s, 1H), 7.70 (dd, J=2.8, 8.8 Hz, 1H), 7.57 (dd, J=1.6, 7.6 Hz, 1H), 7.50-7.33 (m, 2H), 4.84-4.46 (m, 1H), 2.71-2.57 (m, 2H), 2.30-2.16 (m, 5H), 2.06-1.92 (m, 2H), 1.80-1.62 (m, 2H). m/z: [ESI⁺] 429, 431 (M+H)⁺, (C₂₁H₂₁ClN₄O₂S).

### Synthesis of N-(4-(2-chlorophenyl)thiazol-2-yl)-4-(1,1-dioxidotetrahydro-2H-thiopyran-4-yl)benzamide (Compound 382)

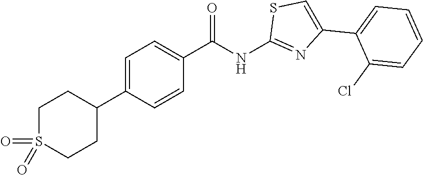 [SMILES: O=C(Nc1nc(-c2ccccc2Cl)cs1)c1ccc(C2CCS(=O)(=O)CC2)cc1]

Compound N-(4-(2-chlorophenyl)thiazol-2-yl)-4-(1,1-dioxidotetrahydro-2H-thiopyran-4-yl)benzamide was prepared from 4-(1,1-dioxidotetrahydro-2H-thiopyran-4-yl)benzoic acid (120 mg, 0.47 mmol) and 4-(2-chlorophenyl)thiazol-2-amine (109 mg, 0.52 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 34 mg (16%). ¹H NMR (400 MHz, DMSO) δ 12.77 (br s, 1H), 8.10 (d, J=8.0 Hz, 2H), 7.91 (dd, J=2.0, 7.6 Hz, 1H), 7.67 (s, 1H), 7.56 (dd, J=1.6, 7.6 Hz, 1H), 7.52-7.26 (m, 4H), 3.37-3.27 (m, 2H), 3.21-3.10 (m, 2H), 3.09-2.98 (m, 1H), 2.23-2.09 (m, 4H). m/z: [ESI⁺] 447, 449 (M+H)⁺, (C₂₁H₁₉ClN₂O₃S₂).

### Synthesis of N-(4-(2-chlorophenyl)thiazol-2-yl)-5-(2-methyl-1-oxo-2,8-diazaspiro[4.5]decan-8-yl)picolinamide (Compound 452)

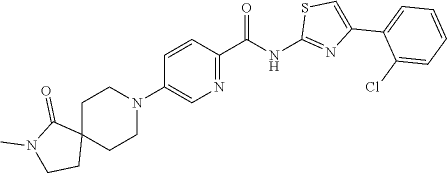 [SMILES: CN1CCC2(CCN(c3ccc(C(=O)Nc4nc(-c5ccccc5Cl)cs4)nc3)CC2)C1=O]

Compound N-(4-(2-chlorophenyl)thiazol-2-yl)-5-(2-methyl-1-oxo-2,8-diazaspiro[4.5]decan-8-yl)picolinamide was prepared from 5-(2-methyl-1-oxo-2,8-diazaspiro[4.5]decan-8-yl)picolinic acid (230 mg, 0.80 mmol) and 4-(2-chlorophenyl)thiazol-2-amine (184 mg, 0.87 mmol), following a procedure similar to that described for the synthesis of methyl (1s,3s)-3-((4-(2-chlorophenyl)thiazol-2-yl)carbamoyl)cyclobutane-1-carboxylate and was isolated as a yellow solid.

Yield 30 mg (8%). ¹H NMR (400 MHz, DMSO) δ 11.57 (br s, 1H), 8.42 (d, J=2.8 Hz, 1H), 8.00 (d, J=8.8 Hz, 1H), 7.93 (d, J=2.0, 7.6 Hz, 1H), 7.71 (s, 1H), 7.62-7.30 (m, 4H), 4.13-3.84 (m, 2H), 3.32-3.26 (m, 211), 3.19-3.05 (m, 2H), 2.74 (s, 3H), 2.05-1.94 (m, 2H), 1.81-1.68 (m, 2H), 1.50-1.40 (m, 2H). m/z: [ESI⁺] 482, 484 (M+H)⁺, (C₂₄H₂₄ClN₅O₂S).

### Synthesis of (1r,3r)-N-(4-(2-chlorophenyl)thiazol-2-yl)-3-morpholinocyclobutane-1-carboxamide (Compound 422)

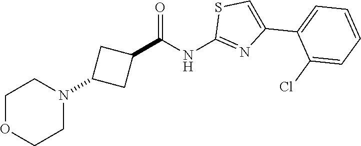 [SMILES: O=C(Nc1nc(-c2ccccc2Cl)cs1)[C@H]1C[C@H](N2CCOCC2)C1]

Compound (1r,3r)-N-(4-(2-chlorophenyl)thiazol-2-yl)-3-morpholinocyclobutane-1-carboxamide was prepared from (1r,3r)-3-morpholinocyclobutane-1-carboxylic acid (500 mg, 2.70 mmol) and 4-(2-chlorophenyl)thiazol-2-amine (626 mg, 2.97 mmol), following a procedure similar to that described for the synthesis of methyl (1s,3s)-3-((4-(2-chlorophenyl)thiazol-2-yl)carbamoyl)cyclobutane-1-carboxylate and was isolated as a light yellow solid.

Yield 25 mg (2%). ¹H NMR (400 MHz, DMSO) δ 12.23 (br s, 1H), 7.82 (dd, J=2.0, 7.6 Hz, 1H), 7.60 (s, 1H), 7.57-7.51 (m, 1H), 7.47-7.31 (m, 2H), 3.62-3.52 (m, 4H), 3.30-3.19 (m, 1H), 2.96-2.82 (m, 1H), 2.35-2.21 (m, 6H), 2.20-2.05 (m, 2H). m/z: [ESI⁺] 378, 380 (M+H)⁺, (C₁₈H₂₀ClN₃O₂S).

### Synthesis of N-(4-(2-chlorophenyl)thiazol-2-yl)-3-morpholinobicyclo[1.1.1]pentane-1-carboxamide (Compound 416)

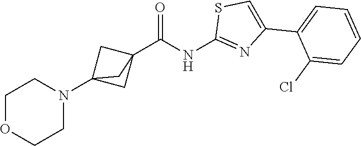 [SMILES: O=C(Nc1nc(-c2ccccc2Cl)cs1)C12CC(N3CCOCC3)(C1)C2]

Compound N-(4-(2-chlorophenyl)thiazol-2-yl)-3-morpholinobicyclo[1.1.1]pentane-1-carboxamide was prepared from 3-morpholinobicyclo[1.1.1]pentane-1-carboxylic acid (200 mg, 1.01 mmol) and 4-(2-chlorophenyl)thiazol-2-amine (214 mg, 1.02 mmol), following a procedure similar to that described for the synthesis of methyl (1s,3s)-3-((4-(2-chlorophenyl)thiazol-2-yl)carbamoyl)cyclobutane-1-carboxylate and was isolated as a light yellow solid.

Yield 27 mg (7%). ¹H NMR (400 MHz, DMSO) δ 12.33 (br s, 1H), 7.85 (dd, J=2.0, 7.6 Hz, 1H), 7.61 (s, 1H), 7.55 (dd, J=1.6, 7.6 Hz, 1H), 7.48-7.32 (m, 2H), 3.73-3.50 (m, 4H), 2.42-2.32 (m, 4H), 2.08 (s, 6H). m/z: [ESI⁺] 390, 392 (M+H)⁺, (C₁₉H₂₀ClN₃O₂S).

### Synthesis of N-(4-(2-chlorophenyl)thiazol-2-yl)-3-(4-(methylsulfonyl)piperazin-1-yl)bicyclo[1.1.1]pentane-1-carboxamide (Compound 418)

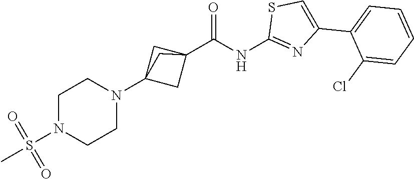 [SMILES: CS(=O)(=O)N1CCN(C23CC(C(=O)Nc4nc(-c5ccccc5Cl)cs4)(C2)C3)CC1]

Compound N-(4-(2-chlorophenyl)thiazol-2-yl)-3-(4-(methylsulfonyl)piperazin-1-yl)bicyclo[1.1.1]pentane-1-carboxamide was prepared from 3-(4-(methylsulfonyl)piperazin-1-yl)bicyclo[1.1.1]pentane-1-carboxylic acid (275 mg, 1.00 mmol) and 4-(2-chlorophenyl)thiazol-2-amine (253 mg, 1.20 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 18 mg (4%). ¹H NMR (400 MHz, DMSO) δ 12.35 (br s, 1H), 7.84 (dd, J=2.0, 7.6 Hz, 1H), 7.61 (s, 1H), 7.55 (dd, J=1.6, 7.6 Hz, 1H), 7.46-7.33 (m, 2H), 3.13 (t, J=5.2 Hz, 4H), 2.88 (s, 3H), 2.52-2.45 (m, 4H), 2.10 (s, 6H). m/z: [ESI⁺] 467, 469 (M+H)⁺, (C₂₀H₂₃ClN₄O₃S₂).

### Synthesis of (1r,3r)-N-(4-(2-chlorophenyl)thiazol-2-yl)-3-(4-(methylsulfonyl)piperazine-1-carbonyl)cyclobutane-1-carboxamide (Compound 430)

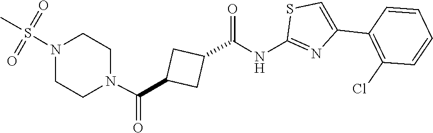 [SMILES: CS(=O)(=O)N1CCN(C(=O)[C@H]2C[C@H](C(=O)Nc3nc(-c4ccccc4Cl)cs3)C2)CC1]

Compound (1r,3r)-N-(4-(2-chlorophenyl)thiazol-2-yl)-3-(4-(methylsulfonyl)piperazine-1-carbonyl)cyclobutane-1-carboxamide was prepared from (1r,3r)-3-((4-(2-chlorophenyl)thiazol-2-yl)carbamoyl)cyclobutane-1-carboxylic acid (200 mg, 0.59 mmol) and 1-(methylsulfonyl)piperazine (98 mg, 0.60 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid. Yield 54 mg (19%). ¹H NMR (400 MHz, DMSO) δ 12.23 (br s, 1H), 7.83 (dd, J=2.0, 7.6 Hz, 1H), 7.61 (s, 1H), 7.56 (dd, J=1.6, 7.6 Hz, 1H), 7.48-7.33 (m, 2H), 3.58 (t, J=5.2 Hz, 2H), 3.45-3.38 (m, 3H), 3.31-3.25 (m, 1H), 3.10 (t, J=5.2 Hz, 4H), 2.89 (s, 3H), 2.46 (t, J=7.6 Hz, 4H). m/z: [ESI⁺] 483, 485 (M+H)⁺, (C₂₀H₂₃ClN₄O₄S₂).

### Synthesis of N-(4-(2-chlorophenyl)thiazol-2-yl)-5-(6-(2-methoxyacetyl)-2,6-diazaspiro[3.3]heptan-2-yl)picolinamide (Compound 450)

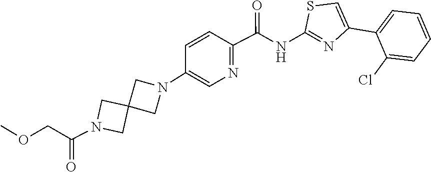 [SMILES: COCC(=O)N1CC2(C1)CN(c1ccc(C(=O)Nc3nc(-c4ccccc4Cl)cs3)nc1)C2]

Compound N-(4-(2-chlorophenyl)thiazol-2-yl)-5-(6-(2-methoxyacetyl)-2,6-diazaspiro[3.3]heptan-2-yl)picolinamide was prepared from 2-methoxyacetic acid (44 mg, 0.49 mmol) and N-(4-(2-chlorophenyl)thiazol-2-yl)-5-(2,6-diazaspiro[3.3]heptan-2-yl)picolinamide 2,2,2-trifluoroacetate salt (263 mg, 0.50 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 20 mg (8%). ¹H NMR (400 MHz, DMSO) δ 11.56 (br s, 1H), 7.99 (d, J=8.8 Hz, 1H), 7.92 (dd, J=2.0, 7.6 Hz, 1H), 7.89 (d, J=2.8 Hz, 1H), 7.70 (s, 1H), 7.57 (dd, J=1.6, 7.6 Hz, 1H), 7.48-7.32 (m, 2H), 6.97 (dd, J=2.8, 8.8 Hz, 1H), 4.38 (s, 2H), 4.21 (s, 4H), 4.12 (s, 2H), 3.90 (s, 2H), 3.28 (s, 3H). m/z: [ESI⁺] 484, 486 (M+H)⁺, (C₂₃H₂₂ClN₅O₃S).

### Synthesis of N-(4-(2-chlorophenyl)thiazol-2-yl)-5-(4-(dimethylglycyl)piperazin-1-yl)picolinamide (Compound 443)

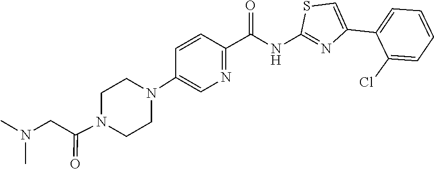 [SMILES: CN(C)CC(=O)N1CCN(c2ccc(C(=O)Nc3nc(-c4ccccc4Cl)cs3)nc2)CC1]

Compound N-(4-(2-chlorophenyl)thiazol-2-yl)-5-(4-(dimethylglycyl)piperazin-1-yl)picolinamide was prepared from dimethylglycine (39 mg, 0.38 mmol) and N-(4-(2-chlorophenyl)thiazol-2-yl)-5-(piperazin-1-yl)picolinamide hydrochloride (175 mg, 0.40 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 9 mg (5%). ¹H NMR (400 MHz, DMSO) δ 11.65 (br s, 1H), 8.43 (s, 1H), 8.03 (d, J=8.8 Hz, 1H), 7.93 (d, J=7.6 Hz, 1H), 7.72 (s, 1H), 7.62-7.32 (m, 4H), 3.74 (s, 2H), 3.63 (s, 2H), 3.54-3.42 (m, 4H), 3.15 (s, 2H), 2.21 (s, 6H). m/z: [ESI⁺] 485, 487 (M+H)⁺, (C₂₃H₂₅ClN₆O₂S).

### Synthesis of N-(4-(2-chlorophenyl)thiazol-2-yl)-5-(4-(3-(dimethylamino)propanoyl)piperazin-1-yl)picolinamide (Compound 444)

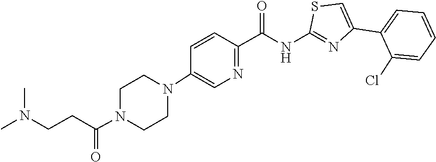 [SMILES: CN(C)CCC(=O)N1CCN(c2ccc(C(=O)Nc3nc(-c4ccccc4Cl)cs3)nc2)CC1]

Compound N-(4-(2-chlorophenyl)thiazol-2-yl)-5-(4-(3-(dimethylamino)propanoyl)piperazin-1-yl)picolinamide was prepared from 3-(dimethylamino)propanoic acid (44 mg, 0.38 mmol) and N-(4-(2-chlorophenyl)thiazol-2-yl)-5-(piperazin-1-yl)picolinamide hydrochloride (175 mg, 0.40 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 8 mg (4%). ¹H NMR (400 MHz, DMSO) δ 11.47 (br s, 1H), 8.42 (d, J=2.8 Hz, 1H), 8.03 (d, J=8.8 Hz, 1H), 7.93 (dd, J=2.0, 7.6 Hz, 1H), 7.71 (s, 1H), 7.57 (dd, J=1.6, 7.6 Hz, 1H), 7.53-7.28 (m, 3H), 3.76-3.58 (m, 4H), 3.57-3.42 (m, 4H), 2.55 (s, 4H), 2.20 (s, 6H). m/z: [ESI⁺] 499, 501 (M+H)⁺, (C₂₄H₂₇ClN₆O₂S).

### Synthesis of N-(4-(2-chlorophenyl)thiazol-2-yl)-5-(4-(2-hydroxyacetyl)piperazin-1-yl)picolinamide (Compound 445)

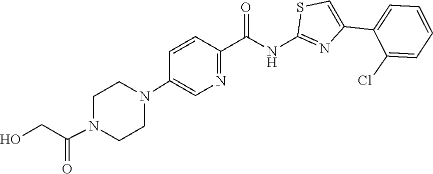 [SMILES: O=C(Nc1nc(-c2ccccc2Cl)cs1)c1ccc(N2CCN(C(=O)CO)CC2)cn1]

Compound N-(4-(2-chlorophenyl)thiazol-2-yl)-5-(4-(2-hydroxyacetyl)piperazin-1-yl)picolinamide was prepared from 2-hydroxyacetic acid (29 mg, 0.38 mmol) and N-(4-(2-chlorophenyl)thiazol-2-yl)-5-(piperazin-1-yl)picolinamide hydrochloride (175 mg, 0.40 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 20 mg (11%). ¹H NMR (400 MHz, DMSO) δ 11.64 (br s, 1H), 8.42 (d, J=2.8 Hz, 1H), 8.02 (d, J=8.8 Hz, 1H), 7.93 (dd, J=2.0, 7.6 Hz, 1H), 7.71 (s, 1H), 7.56 (dd, J=1.6, 7.6 Hz, 1H), 7.52-7.35 (m, 3H), 4.70 (t, J=5.6 Hz, 1H), 4.16 (d, J=5.6 Hz, 2H), 3.70-3.60 (m, 2H), 3.58-3.46 (m, 6H). m/z: [ESI⁺] 458, 460 (M+H)⁺, (C₂₁H₂₀ClN₅O₃S).

### Synthesis of N-(4-(2-chlorophenyl)thiazol-2-yl)-5-(4-(methylprolyl)piperazin-1-yl)picolinamide (Compound 466)

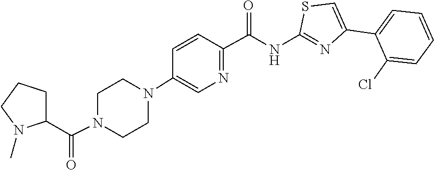 [SMILES: CN1CCCC1C(=O)N1CCN(c2ccc(C(=O)Nc3nc(-c4ccccc4Cl)cs3)nc2)CC1]

Compound N-(4-(2-chlorophenyl)thiazol-2-yl)-5-(4-(methylprolyl)piperazin-1-yl)picolinamide was prepared from methylproline (30 mg, 0.23 mmol) and N-(4-(2-chlorophenyl)thiazol-2-yl)-5-(piperazin-1-yl)picolinamide hydrochloride (111 mg, 0.25 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 17 mg (14%). ¹H NMR (400 MHz, DMSO) δ 11.65 (br s, 1H), 8.43 (d, J=2.8 Hz, 1H), 8.03 (d, J=8.8 Hz, 1H), 7.93 (dd, J=2.0, 7.6 Hz, 1H), 7.72 (s, 1H), 7.57 (dd, J=1.6, 8.0 Hz, 1H), 7.53-7.33 (m, 3H), 3.98-3.85 (m, 1H), 3.80-3.69 (m, 2H), 3.63-3.49 (m, 4H), 3.08-2.99 (m, 2H), 2.30-2.20 (m, 4H), 2.17-2.06 (m, 2H), 1.82-1.70 (m, 3H). m/z: [ESI⁺] 511, 513 (M+H)⁺, (C₂₅H₂₇ClN₆O₂S).

### Synthesis of N-(4-(2-chlorophenyl)thiazol-2-yl)-5-(4-(1-methylpyrrolidine-3-carbonyl)piperazin-1-yl)picolinamide (Compound 482)

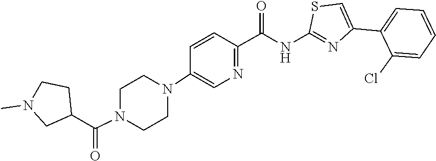 [SMILES: CN1CCC(C(=O)N2CCN(c3ccc(C(=O)Nc4nc(-c5ccccc5Cl)cs4)nc3)CC2)C1]

Compound N-(4-(2-chlorophenyl)thiazol-2-yl)-5-(4-(1-methylpyrrolidine-3-carbonyl)piperazin-1-yl)picolinamide was prepared from 1-methylpyrrolidine-3-carboxylic acid (80 mg, 0.62 mmol) and N-(4-(2-chlorophenyl)thiazol-2-yl)-5-(piperazin-1-yl)picolinamide hydrochloride (324 mg, 0.74 mmol), following a procedure similar to that described for the synthesis of methyl (1s,3s)-3-((4-(2-chlorophenyl)thiazol-2-yl)carbamoyl)cyclobutane-1-carboxylate and was isolated as a grey solid.

Yield 5 mg (2%). ¹H NMR (400 MHz, DMSO) δ 11.63 (br s, 1H), 8.43 (s, 1H), 8.03 (d, J=8.8 Hz, 1H), 7.93 (d, J=7.6 Hz, 1H), 7.71 (s, 1H), 7.57 (d, J=8.0 Hz, 1H), 7.52-7.48 (m, 1H), 7.47-7.35 (m, 2H), 3.73-3.59 (m, 4H), 3.53-3.41 (m, 4H), 3.30-3.25 (m, 1H), 2.75 (t, J=8.8 Hz, 1H), 2.59-2.52 (m, 1H), 2.47-2.44 (m, 111), 2.37-2.30 (m, 1H), 2.23 (s, 3H), 2.01-1.90 (m, 2H). m/z: [ESI⁺] 511, 513 (M+H)⁺, (C₂₅H₂₇ClN₆O₂S).

### Synthesis of N-(4-(2-chlorophenyl)thiazol-2-yl)-5-(4-(1-methylpiperidine-4-carbonyl)piperazin-1-yl)picolinamide (Compound 483)

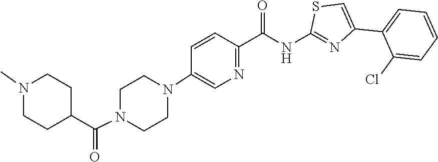 [SMILES: CN1CCC(C(=O)N2CCN(c3ccc(C(=O)Nc4nc(-c5ccccc5Cl)cs4)nc3)CC2)CC1]

Compound N-(4-(2-chlorophenyl)thiazol-2-yl)-5-(4-(1-methylpiperidine-4-carbonyl)piperazin-1-yl)picolinamide was prepared from 1-methylpiperidine-4-carboxylic acid (80 mg, 0.56 mmol) and N-(4-(2-chlorophenyl)thiazol-2-yl)-5-(piperazin-1-yl)picolinamide hydrochloride (292 mg, 0.67 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 17 mg (6%). ¹H NMR (400 MHz, DMSO) δ 11.64 (br s, 1H), 8.42 (s, 1H), 8.03 (d, J=8.8 Hz, 1H), 7.93 (d, J=7.6 Hz, 1H), 7.71 (s, 1H), 7.57 (d, J=7.6 Hz, 1H), 7.52-7.36 (m, 3H), 3.73-3.61 (m, 4H), 3.50-3.43 (m, 4H), 2.83-2.74 (m, 2H), 2.63-2.54 (m, 1H), 2.16 (s, 3H), 1.97-1.84 (m, 2H), 1.66-1.53 (m, 4H). m/z: [ESI⁺] 525, 527 (M+H)⁺, (C₂₆H₂₉ClN₆O₂S).

### Synthesis of N-(4-(2-chlorophenyl)thiazol-2-yl)-5-(4-(1-methylpiperidine-3-carbonyl)piperazin-1-yl)picolinamide hemiformate (Compound 467)

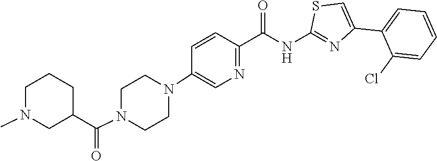 [SMILES: CN1CCCC(C(=O)N2CCN(c3ccc(C(=O)Nc4nc(-c5ccccc5Cl)cs4)nc3)CC2)C1]

Compound N-(4-(2-chlorophenyl)thiazol-2-yl)-5-(4-(1-methylpiperidine-3-carbonyl)piperazin-1-yl)picolinamide hemiformate was prepared from 1-methylpiperidine-3-carboxylic acid (150 mg, 1.05 mmol) and N-(4-(2-chlorophenyl)thiazol-2-yl)-5-(piperazin-1-yl)picolinamide hydrochloride (548 mg, 1.26 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 60 mg (11%). ¹H NMR (400 MHz, DMSO) δ 11.64 (br s, 1H), 8.42 (d, J=2.8 Hz, 1H), 8.21 (s, 0.4H, HCOOH), 8.03 (d, J=8.8 Hz, 1H), 7.93 (dd, J=2.0, 7.6 Hz, 111), 7.71 (s, 1H), 7.60-7.53 (m, 1H), 7.52-7.35 (m, 3H), 3.69-3.62 (m, 4H), 3.51-3.43 (m, 4H), 2.93-2.83 (m, 1H), 2.79 (dd, J=3.2, 11.2 Hz, 2H), 2.21 (s, 3H), 2.03 (t, J=10.8 Hz, 1H), 1.94-1.83 (m, 1H), 1.76-1.69 (m, 1H), 1.67-1.52 (m, 2H), 1.37-1.23 (m, 1H). m/z: [ESI⁺] 525, 527 (M+H)⁺, (C₂₆H₂₉ClN₆O₂S).

### Synthesis of (R)—N-(4-(2-chlorophenyl)thiazol-2-yl)-5-(4-(1-methylpiperidine-3-carbonyl)piperazin-1-yl)picolinamide (Compound 485)

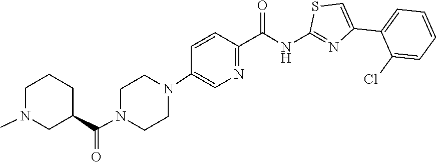 [SMILES: CN1CCC[C@@H](C(=O)N2CCN(c3ccc(C(=O)Nc4nc(-c5ccccc5Cl)cs4)nc3)CC2)C1]

Compound (R)—N-(4-(2-chlorophenyl)thiazol-2-yl)-5-(4-(1-methylpiperidine-3-carbonyl)piperazin-1-yl)picolinamide was prepared from (R)-1-methylpiperidine-3-carboxylic acid (250 mg, 1.75 mmol) and N-(4-(2-chlorophenyl)thiazol-2-yl)-5-(piperazin-1-yl)picolinamide hydrochloride (991 mg, 2.27 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 300 mg (33%). ¹H NMR (400 MHz, DMSO) δ 11.65 (br s, 1H), 8.42 (d, J=2.8 Hz, 1H), 8.03 (d, J=8.8 Hz, 1H), 7.93 (dd, J=2.0, 7.6 Hz, 1H), 7.72 (s, 1H), 7.57 (dd, J=1.6, 7.6 Hz, 1H), 7.53-7.35 (m, 3H), 3.71-3.59 (m, 4H), 3.52-3.41 (m, 4H), 2.89-2.79 (m, 1H), 2.75 (d, J=11.2 Hz, 2H), 2.17 (s, 3H), 1.93 (t, J=11.2 Hz, 1H), 1.84-1.76 (m, 1H), 1.75-1.68 (m, 111), 1.66-1.53 (m, 2H), 1.35-1.21 (m, 1H). m/z: [ESI⁺] 525, 527 (M+H)⁺, (C₂₆H₂₉ClN₆O₂S).

### Synthesis of (S)—N-(4-(2-chlorophenyl)thiazol-2-yl)-5-(4-(1-methylpiperidine-3-carbonyl)piperazin-1-yl)picolinamide (Compound 486)

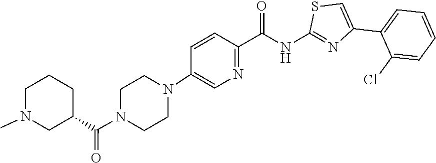 [SMILES: CN1CCC[C@H](C(=O)N2CCN(c3ccc(C(=O)Nc4nc(-c5ccccc5Cl)cs4)nc3)CC2)C1]

Compound (S)—N-(4-(2-chlorophenyl)thiazol-2-yl)-5-(4-(1-methylpiperidine-3-carbonyl)piperazin-1-yl)picolinamide was prepared from (S)-1-methylpiperidine-3-carboxylic acid (0.25 g, 1.75 mmol) and N-(4-(2-chlorophenyl)thiazol-2-yl)-5-(piperazin-1-yl)picolinamide hydrochloride (1.07 g, 2.45 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 217 mg (24%). ¹H NMR (400 MHz, DMSO) δ 11.64 (br s, 1H), 8.42 (d, J=2.8 Hz, 1H), 8.03 (d, J=8.8 Hz, 1H), 7.93 (dd, J=2.0, 7.6 Hz, 1H), 7.71 (s, 1H), 7.57 (dd, J=1.6, 7.6 Hz, 1H), 7.53-7.35 (m, 311), 3.71-3.58 (m, 4H), 3.53-3.41 (m, 4H), 2.89-2.79 (m, 1H), 2.75 (d, J=11.2 Hz, 2H), 2.17 (s, 3H), 1.93 (t, J=11.2 Hz, 1H), 1.84-1.68 (m, 2H), 1.67-1.49 (m, 2H), 1.35-1.21 (m, 1H). m/z: [ESI⁺] 525, 527 (M+H)⁺, (C₂₆H₂₉ClN₆O₂S).

### Synthesis of N-(4-(2-chlorophenyl)thiazol-2-yl)-5-(2-oxa-7-azaspiro[3.5]nonan-7-yl)picolinamide (Compound 448)

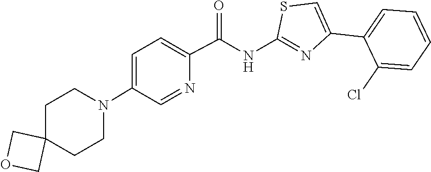 [SMILES: O=C(Nc1nc(-c2ccccc2Cl)cs1)c1ccc(N2CCC3(CC2)COC3)cn1]

Compound N-(4-(2-chlorophenyl)thiazol-2-yl)-5-(2-oxa-7-azaspiro[3.5]nonan-7-yl)picolinamide was prepared from 5-(2-oxa-7-azaspiro[3.5]nonan-7-yl)picolinic acid (500 mg, 2.01 mmol) and 4-(2-chlorophenyl)thiazol-2-amine (509 mg, 2.42 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 25 mg (3%). ¹H NMR (400 MHz, DMSO) δ 11.57 (br s, 1H), 8.42 (d, J=2.8 Hz, 1H), 7.98 (d, J=8.8 Hz, 1H), 7.93 (dd, J=2.0, 7.6 Hz, 1H), 7.71 (s, 1H), 7.57 (dd, J=1.6, 7.6 Hz, 1H), 7.53-7.35 (m, 3H), 4.37 (s, 4H), 3.44-3.38 (m, 4H), 1.93-1.85 (m, 4H). m/z: [ESI⁺] 441, 443 (M+H)⁺, (C₂₂H₂₁ClN₄O₂S).

### Synthesis of N-(4-(2-chlorophenyl)thiazol-2-yl)-5-(4-hydroxypiperidin-1-yl)picolinamide (Compound 446)

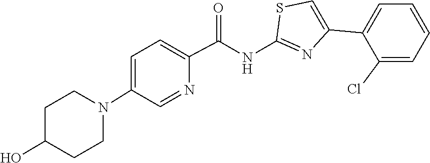 [SMILES: O=C(Nc1nc(-c2ccccc2Cl)cs1)c1ccc(N2CCC(O)CC2)cn1]

Compound N-(4-(2-chlorophenyl)thiazol-2-yl)-5-(4-hydroxypiperidin-1-yl)picolinamide was prepared from 5-(4-((tert-butyldimethylsilyl)oxy)piperidin-1-yl)picolinic acid (650 mg, 1.93 mmol) and 4-(2-chlorophenyl)thiazol-2-amine (448 mg, 2.13 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 30 mg (4%). ¹H NMR (400 MHz, DMSO) δ 8.41 (d, J=2.8 Hz, 1H), 7.99 (d, J=8.8 Hz, 1H), 7.94 (dd, J=2.0, 7.6 Hz, 1H), 7.71 (s, 1H), 7.57 (dd, J=1.6, 7.6 Hz, 1H), 7.50-7.37 (m, 3H), 4.83-4.70 (m, 1H), 3.86-3.78 (m, 2H), 3.78-3.73 (m, 1H), 3.23-3.13 (m, 2H), 1.90-1.80 (m, 2H), 1.53-1.41 (m, 2H). Amide NH not observed (TBDMSO was cleaved during the reaction). m/z: [ESI⁺] 415,417 (M+H)⁺, (C₂₀H₁₉ClN₄O₂S).

### Synthesis of N-(4-(2-(methoxymethyl)phenyl)thiazol-2-yl)-5-(4-(methylsulfonyl)piperazin-1-yl)picolinamide (Compound 447)

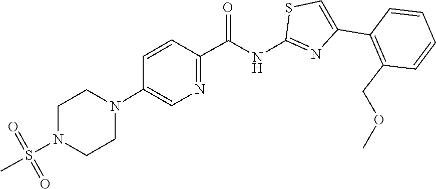 [SMILES: COCc1ccccc1-c1csc(NC(=O)c2ccc(N3CCN(S(C)(=O)=O)CC3)cn2)n1]

Compound N-(4-(2-(methoxymethyl)phenyl)thiazol-2-yl)-5-(4-(methylsulfonyl)piperazin-1-yl)picolinamide was prepared from 5-(4-(methylsulfonyl)piperazin-1-yl)picolinic acid (236 mg, 0.83 mmol) and 4-(2-(methoxymethyl)phenyl)thiazol-2-amine (200 mg, 0.91 mmol), following a procedure similar to that described for the synthesis of methyl (1s,3s)-3-((4-(2-chlorophenyl)thiazol-2-yl)carbamoyl)cyclobutane-1-carboxylate and was isolated as an off-white solid.

Yield 73 mg (18%). ¹H NMR (400 MHz, DMSO) δ 11.63 (br s, 1H), 8.46 (d, J=2.8 Hz, 1H), 8.04 (d, J=8.8 Hz, 1H), 7.77-7.64 (m, 1H), 7.57-7.47 (m, 2H), 7.42-7.34 (m, 3H), 4.61 (s, 2H), 3.54-3.60 (m, 4H), 3.35 (s, 3H), 3.25-3.31 (m, 4H), 2.94 (s, 3H). m/z: [ESI⁺] 488 (M+H)⁺, (C₂₂H₂₅N₅O₄S₂).

### Synthesis of 5-morpholino-N-(4-(2-oxopyrrolidin-1-yl)thiazol-2-yl)picolinamide (Compound 395)

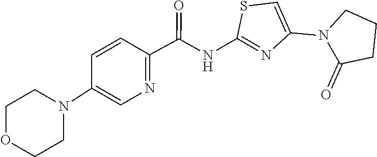 [SMILES: O=C(Nc1nc(N2CCCC2=O)cs1)c1ccc(N2CCOCC2)cn1]

Compound 5-morpholino-N-(4-(2-oxopyrrolidin-1-yl)thiazol-2-yl)picolinamide was prepared from 5-morpholinopicolinic acid (89 mg, 0.43 mmol) and 1-(2-aminothiazol-4-yl)pyrrolidin-2-one (60 mg, 0.33 mmol), following a procedure similar to that described for the synthesis of tert-butyl 4-(6-((4-(2-chlorophenyl)thiazol-2-yl)carbamoyl)pyridin-3-yl)piperazine-1-carboxylate and was isolated as an off-white solid.

Yield 13 mg (11%). ¹H NMR (400 MHz, DMSO) δ 11.48 (br s, 1H), 8.40 (d, J=2.8 Hz, 1H), 8.00 (d, J=8.8 Hz, 1H), 7.49 (dd, J=2.8, 8.8 Hz, 1H), 7.31 (s, 1H), 3.98 (t, J=7.2 Hz, 2H), 3.81-3.74 (m, 4H), 3.43-3.36 (m, 4H), 2.50-2.46 (m, 2H), 2.14-2.02 (m, 2H). m/z: [ESI⁺] 374 (M+H)⁺, (C₁₇H₁₉N₅O₃S).

### Synthesis of N-(4-(2-chlorophenyl)-1H-imidazol-2-yl)-5-morpholinopicolinamide (Compound 404)

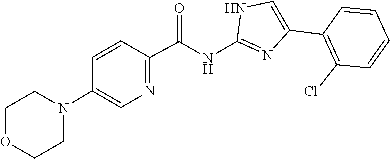 [SMILES: O=C(Nc1nc(-c2ccccc2Cl)c[nH]1)c1ccc(N2CCOCC2)cn1]

Compound N-(4-(2-chlorophenyl)-1H-imidazol-2-yl)-5-morpholinopicolinamide was prepared from 5-morpholinopicolinic acid (210 mg, 1.01 mmol) and 4-(2-chlorophenyl)-1H-imidazol-2-amine (130 mg, 0.67 mmol), following a procedure similar to that described for the synthesis of tert-butyl 4-(6-((4-(2-chlorophenyl)thiazol-2-yl)carbamoyl)pyridin-3-yl)piperazine-1-carboxylate and was isolated as an off-white solid.

Yield 10 mg (4%). ¹H NMR (400 MHz, DMSO) δ 12.06 (br s, 1H), 10.67 (br s, 1H), 8.41 (d, J=2.8 Hz, 1H), 8.10 (d, J=7.6 Hz, 1H), 8.01 (d, J=8.8 Hz, 1H), 7.54 (s, 1H), 7.51-7.44 (m, 2H), 7.41-7.33 (m, 1H), 7.27-7.18 (m, 1H), 3.74-3.80 (m, 4H), 3.36-3.40 (m, 4H). m/z: [ESI⁺] 384, 386 (M+H)⁺, (C₁₉H₁₈ClN₅O₂).

### Synthesis of 2-chloro-N-(4-(2-chlorophenyl)thiazol-2-yl)-4-morpholinobenzamide (Compound 409)

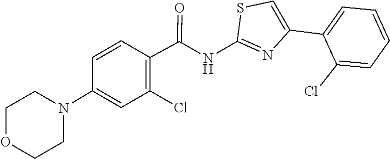 [SMILES: O=C(Nc1nc(-c2ccccc2Cl)cs1)c1ccc(N2CCOCC2)cc1Cl]

Compound 2-chloro-N-(4-(2-chlorophenyl)thiazol-2-yl)-4-morpholinobenzamide was prepared from 2-chloro-4-morpholinobenzoic acid (400 mg, 1.66 mmol) and 4-(2-chlorophenyl)thiazol-2-amine (350 mg, 1.66 mmol), following a procedure similar to that described for the synthesis of tert-butyl 4-(6-((4-(2-chlorophenyl)thiazol-2-yl)carbamoyl)pyridin-3-yl)piperazine-1-carboxylate and was isolated as a yellow solid.

Yield 98 mg (14%). ¹H NMR (400 MHz, DMSO) δ 12.61 (br s, 1H), 7.86 (dd, J=2.0, 7.6 Hz, 1H), 7.66 (s, 1H), 7.60-7.53 (m, 2H), 7.48-7.34 (m, 2H), 7.05 (d, J=2.4 Hz, 1H), 6.97 (dd, J=2.4, 8.8 Hz, 1H), 3.77-3.70 (m, 4H), 3.30-3.23 (m, 4H). m/z: [ESI⁺] 434, 436 (M+H)⁺, (C₂₀H₁₇C₂N₃O₂S).

### Synthesis of N-(4-(2-chlorophenyl)thiazol-2-yl)-2-methyl-4-morpholinobenzamide (Compound 410)

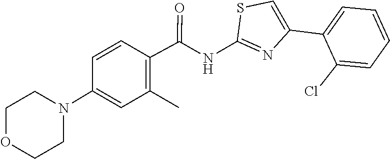 [SMILES: Cc1cc(N2CCOCC2)ccc1C(=O)Nc1nc(-c2ccccc2Cl)cs1]

Compound N-(4-(2-chlorophenyl)thiazol-2-yl)-2-methyl-4-morpholinobenzamide was prepared from 2-methyl-4-morpholinobenzoic acid (500 mg, 2.26 mmol) and 4-(2-chlorophenyl)thiazol-2-amine (477 mg, 2.26 mmol), following a procedure similar to that described for the synthesis of tert-butyl 4-(6-((4-(2-chlorophenyl)thiazol-2-yl)carbamoyl)pyridin-3-yl)piperazine-1-carboxylate and was isolated as a yellow solid.

Yield 10 mg (1%). ¹H NMR (400 MHz, DMSO) δ 12.39 (br s, 1H), 7.88 (dd, J=1.6, 7.6 Hz, 1H), 7.62 (s, 1H), 7.60-7.53 (m, 2H), 7.48-7.34 (m, 2H), 6.89-6.79 (m, 2H), 3.68-3.77 (m, 4H), 3.19-3.28 (m, 4H), 2.46 (s, 3H). m/z: [ESI⁺] 414, 416 (M+H)⁺, (C₂₁H₂₀ClN₃O₂S).

### Synthesis of N-(4-(2-chlorophenyl)thiazol-2-yl)-4-morpholino-2-(trifluoromethyl)benzamide (Compound 411)

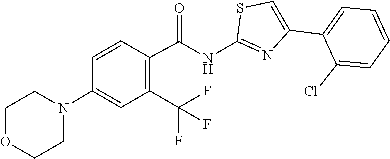 [SMILES: O=C(Nc1nc(-c2ccccc2Cl)cs1)c1ccc(N2CCOCC2)cc1C(F)(F)F]

Compound N-(4-(2-chlorophenyl)thiazol-2-yl)-4-morpholino-2-(trifluoromethyl)benzamide was prepared from 4-morpholino-2-(trifluoromethyl)benzoic acid (335 mg, 1.22 mmol) and 4-(2-chlorophenyl)thiazol-2-amine (257 mg, 1.22 mmol), following a procedure similar to that described for the synthesis of tert-butyl 4-(6-((4-(2-chlorophenyl)thiazol-2-yl)carbamoyl)pyridin-3-yl)piperazine-1-carboxylate and was isolated as a yellow solid.

Yield 10 mg (2%). ¹H NMR (400 MHz, DMSO) δ 12.79 (br s, 1H), 7.86 (dd, J=2.0, 7.6 Hz, 1H), 7.69-7.60 (m, 2H), 7.57 (dd, J=1.6, 7.6 Hz, 1H), 7.48-7.34 (m, 2H), 7.28 (d, J=2.4 Hz, 1H), 7.24 (dd, J=2.4, 8.8 Hz, 1H), 3.79-3.73 (m, 4H), 3.33-3.29 (m, 4H). ¹⁹F NMR (376 MHz, DMSO) δ −57.66. m/z: [ESI⁺] 468, 470 (M+H)⁺, (C₂₁H₁₇ClF₃N₃O₂S).

### Synthesis of N-(4-(2-chlorophenyl)thiazol-2-yl)-4-(tetrahydro-2H-pyran-4-yl)benzamide (Compound 380)

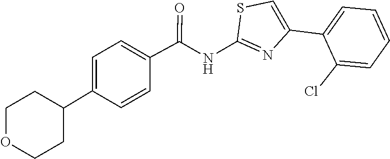 [SMILES: O=C(Nc1nc(-c2ccccc2Cl)cs1)c1ccc(C2CCOCC2)cc1]

Compound N-(4-(2-chlorophenyl)thiazol-2-yl)-4-(tetrahydro-2H-pyran-4-yl)benzamide was prepared from 4-(tetrahydro-2H-pyran-4-yl)benzoic acid (200 mg, 0.97 mmol) and 4-(2-chlorophenyl)thiazol-2-amine (204 mg, 0.97 mmol), following a procedure similar to that described for the synthesis of tert-butyl 4-(6-((4-(2-chlorophenyl)thiazol-2-yl)carbamoyl)pyridin-3-yl)piperazine-1-carboxylate and was isolated as an off-white solid.

Yield 184 mg (48%). ¹H NMR (400 MHz, DMSO) δ 12.77 (br s, 1H), 8.09 (d, J=8.4 Hz, 2H), 7.91 (dd, J=2.0, 7.6 Hz, 1H), 7.68 (s, 1H), 7.57 (dd, J=1.6, 8.0 Hz, 1H), 7.50-7.35 (m, 4H), 4.01-3.92 (m, 2H), 3.52-3.37 (m, 2H), 2.94-2.81 (m, 1H), 1.76-1.63 (m, 4H). m/z: [ESI⁺] 399, 401 (M+H)⁺, (C₂₁H₁₉ClN₂O₂S).

### Synthesis of N-(4-(2-chlorophenyl)thiazol-2-yl)-5-(tetrahydro-2H-pyran-4-yl)picolinamide (Compound 381)

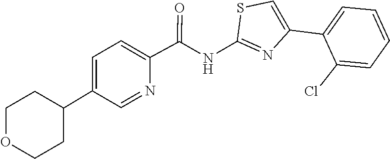 [SMILES: O=C(Nc1nc(-c2ccccc2Cl)cs1)c1ccc(C2CCOCC2)cn1]

Compound N-(4-(2-chlorophenyl)thiazol-2-yl)-5-(tetrahydro-2H-pyran-4-yl)picolinamide was prepared from 5-(tetrahydro-2H-pyran-4-yl)picolinic acid (100 mg, 0.48 mmol) and 4-(2-chlorophenyl)thiazol-2-amine (122 mg, 0.58 mmol), following a procedure similar to that described for the synthesis of tert-butyl 4-(6-((4-(2-chlorophenyl)thiazol-2-yl)carbamoyl)pyridin-3-yl)piperazine-1-carboxylate and was isolated as an off-white solid.

Yield 10 mg (5%). ¹H NMR (400 MHz, DMSO) δ 12.03 (br s, 1H), 8.71 (d, J=2.0 Hz, 1H), 8.15 (d, J=8.0 Hz, 1H), 8.02 (dd, J=2.0, 8.0 Hz, 1H), 7.93 (dd, J=2.0, 7.6 Hz, 1H), 7.76 (s, 1H), 7.58 (dd, J=1.6, 7.6 Hz, 1H), 7.50-7.36 (m, 2H), 4.05-3.92 (m, 2H), 3.51-3.43 (m, 2H), 3.06-2.96 (m, 1H), 1.81-1.72 (m, 4H). m/z: [ESI⁺] 400, 402 (M+H)⁺, (C₂₀H₁₈ClN₃O₂S).

### Synthesis of N-(4-(2-chlorophenyl)thiazol-2-yl)-5-(1,1-dioxidotetrahydro-2H-thiopyran-4-yl)picolinamide (Compound 383)

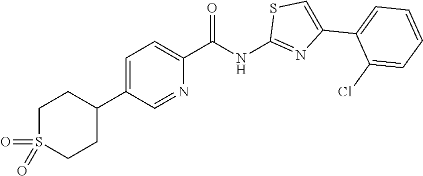 [SMILES: O=C(Nc1nc(-c2ccccc2Cl)cs1)c1ccc(C2CCS(=O)(=O)CC2)cn1]

Compound N-(4-(2-chlorophenyl)thiazol-2-yl)-5-(1,1-dioxidotetrahydro-2H-thiopyran-4-yl)picolinamide was prepared from 5-(1,1-dioxidotetrahydro-2H-thiopyran-4-yl)picolinic acid (50 mg, 0.20 mmol) and 4-(2-chlorophenyl)thiazol-2-amine (45 mg, 0.21 mmol), following a procedure similar to that described for the synthesis of tert-butyl 4-(6-((4-(2-chlorophenyl)thiazol-2-yl)carbamoyl)pyridin-3-yl)piperazine-1-carboxylate and was isolated as an off-white solid.

Yield 21 mg (24%). ¹H NMR (400 MHz, DMSO) δ 12.08 (br s, 1H), 8.71 (d, J=2.0 Hz, 1H), 8.16 (d, J=8.0 Hz, 1H), 8.04 (dd, J=2.0, 8.0 Hz, 1H), 7.93 (dd, J=2.0, 7.6 Hz, 1H), 7.76 (s, 1H), 7.57 (dd, J=1.6, 8.0 Hz, 1H), 7.50-7.35 (m, 2H), 3.42-3.31 (m, 2H), 3.24-3.11 (m, 3H), 2.29-2.15 (m, 4H). m/z: [ESI⁺] 448, 450 (M+H)⁺, (C₂₀H₁₈ClN₃O₃S₂).

### Synthesis of N-(4-(2-chlorophenyl)thiazol-2-yl)-2-methoxy-4-morpholinobenzamide (Compound 441)

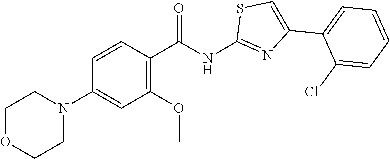 [SMILES: COc1cc(N2CCOCC2)ccc1C(=O)Nc1nc(-c2ccccc2Cl)cs1]

Compound N-(4-(2-chlorophenyl)thiazol-2-yl)-2-methoxy-4-morpholinobenzamide was prepared from 2-methoxy-4-morpholinobenzoic acid (150 mg, 0.63 mmol) and 4-(2-chlorophenyl)thiazol-2-amine (133 mg, 0.63 mmol), following a procedure similar to that described for the synthesis of tert-butyl 4-(6-((4-(2-chlorophenyl)thiazol-2-yl)carbamoyl)pyridin-3-yl)piperazine-1-carboxylate and was isolated as an off-white solid.

Yield 60 mg (22%). ¹H NMR (400 MHz, DMSO) δ 11.26 (br s, 1H), 7.89 (dd, J=2.0, 7.6 Hz, 1H), 7.84 (d, J=8.8 Hz, 1H), 7.64 (s, 1H), 7.56 (dd, J=1.6, 7.6 Hz, 1H), 7.49-7.35 (m, 2H), 6.71 (dd, J=2.0, 8.8 Hz, 1H), 6.63 (d, J=2.4 Hz, 1H), 4.03 (s, 3H), 3.79-3.72 (m, 4H), 3.37-3.35 (m, 4H). m/z: [ESI⁺] 430, 432 (M+H)⁺, (C₂₁H₂₀ClN₃O₃S).

### Example 2

### Biological Activity of Compounds of the Invention

The biological activity results of all compounds of the invention is summarized in Table 2.

TABLE 2
Cellular EC₅₀ values of compounds of the invention
in the WI-38 collagen 1 inhibition assay.

| | COL1 Efficacy |
|---|---|
| | (LogEC₅₀) |
| | −: inactive |
| | +: >−5 |
| Compound | ++: −5 to −6 |
| No. | +++: <−6 |
| 300 | ++ |
| 301 | +++ |
| 302 | ++ |
| 303 | + |
| 304 | +++ |
| 305 | ++ |
| 306 | ++ |
| 307 | +++ |
| 308 | ++ |
| 310 | +++ |
| 311 | ++ |
| 312 | ++ |
| 313 | +++ |
| 314 | ++ |
| 315 | +++ |
| 316 | ++ |
| 317 | + |
| 318 | +++ |
| 319 | ++ |
| 320 | ++ |
| 321 | +++ |
| 322 | +++ |
| 323 | + |
| 324 | ++ |
| 325 | +++ |
| 326 | ++ |
| 327 | +++ |
| 328 | ++ |
| 329 | ++ |
| 330 | ++ |
| 331 | + |
| 332 | ++ |
| 333 | ++ |
| 334 | +++ |
| 335 | + |
| 336 | ++ |
| 337 | ++ |
| 338 | + |
| 339 | +++ |
| 340 | +++ |
| 341 | ++ |
| 342 | ++ |
| 343 | +++ |
| 344 | +++ |
| 345 | +++ |
| 346 | +++ |
| 347 | ++ |
| 348 | + |
| 349 | − |
| 350 | − |
| 351 | +++ |
| 352 | + |
| 353 | +++ |
| 354 | ++ |
| 355 | − |
| 356 | − |
| 357 | + |
| 358 | − |
| 359 | + |
| 360 | + |
| 361 | − |
| 363 | ++ |
| 365 | +++ |
| 366 | +++ |
| 367 | +++ |
| 368 | ++ |
| 369 | ++ |
| 370 | + |
| 371 | ++ |
| 372 | + |
| 373 | + |
| 374 | + |
| 375 | + |
| 377 | +++ |
| 378 | + |
| 376 | +++ |
| 379 | ++ |
| 380 | +++ |
| 381 | +++ |
| 382 | +++ |
| 383 | +++ |
| 384 | ++ |
| 385 | ++ |
| 389 | ++ |
| 393 | ++ |
| 395 | + |
| 399 | ++ |
| 400 | ++ |
| 403 | +++ |
| 404 | +++ |
| 408 | − |
| 409 | ++ |
| 410 | +++ |
| 411 | +++ |
| 416 | ++ |
| 418 | ++ |
| 420 | + |
| 421 | + |
| 422 | ++ |
| 423 | − |
| 426 | − |
| 427 | − |
| 429 | + |
| 430 | + |
| 431 | − |
| 432 | − |
| 434 | + |
| 435 | ++ |
| 436 | − |
| 437 | ++ |
| 438 | +++ |
| 439 | +++ |
| 440 | +++ |
| 441 | ++ |
| 442 | +++ |
| 443 | +++ |
| 444 | +++ |
| 445 | +++ |
| 446 | +++ |
| 447 | +++ |
| 448 | +++ |
| 449 | +++ |
| 450 | +++ |
| 451 | ++ |
| 452 | +++ |
| 455 | +++ |
| 456 | +++ |
| 457 | − |
| 458 | +++ |
| 459 | − |
| 460 | ++ |
| 461 | +++ |
| 462 | ++ |
| 463 | +++ |
| 464 | +++ |
| 465 | − |
| 466 | +++ |
| 467 | +++ |
| 468 | +++ |
| 469 | +++ |
| 470 | +++ |
| 471 | +++ |
| 472 | ++ |
| 473 | +++ |
| 474 | − |
| 475 | +++ |
| 476 | − |
| 477 | +++ |
| 478 | +++ |
| 479 | +++ |
| 480 | − |
| 481 | +++ |
| 482 | +++ |
| 483 | +++ |
| 484 | +++ |
| 485 | +++ |
| 486 | +++ |
| 487 | +++ |
| 488 | +++ |

### Example 3

### Experimental Methods

### High Content Screen for the Identification of Collagen I Modulators

Compound effect on translation of Collagen I in W138, human lung fibroblast cell line was conducted using specific PSM assay using tRNAgly and tRNApro isoacceptors, as described herein below. A library of diverse small molecules, 90,000 compounds, was used at a final concentration of 30 uM. Image and data analyses were conducted using Anima's proprietary algorithms. False positive and toxic compounds were eliminated. Compounds which increased or decreased the FRET signal generated by ribosomes during collagen I translation were identified as hits.

Positive hits were re-screened in the specific PSM assay, using tRNAPro and tRNAGly, and counter-screened to eliminate general translation inhibitors in bulk tRNA PSM assay and in a metabolic labeling assay [Click-IT™, L-Azidohomoalanine (AHA)]; collagen-specific regulators were assays using anti-Collagen I immunofluorescence; all assays were run on activated WI38 cells. Hits were scored using Anima's proprietary algorithms, and 360 compounds which selectively inhibited specific PSM assay and reduced collagen I as detected by immunofluorescence were selected as confirmed hits. These compounds were purchased as powder to confirm activity. Re-purchased hits were tested in the specific PSM assay (tRNApro-tRNAgly) and anti-Collagen I immunofluorescence, and in counter assays to eliminate global translation modulators: (1) bulk tRNA and metabolic labeling using Click-IT™ AHA (L-Azidohomoalanine).

### Cell Culture

WI-38 cells (ATCC® CCL-75™) were maintained in MEM EAGLE (NEAA) W. GLUTAMIN (Biological Industries, Cat. 06-1040-15-1A) containing 10% fetal bovine serum (FBS) and 1% Penicillin-Streptomycin Solution. To synchronize the cells (cell cycle synchronization) prior to induction of collagen synthesis, the cells were starved using DMEM-low glucose supplemented with 0.25% FBS for two hours and then without FBS for 24 hours. To induce collagen synthesis, the cells were treated with a collagen induction cocktail for the indicated time. Compounds were added with induction.

Primary human pulmonary fibroblasts (HPF, PromoCell C-12360) were maintained in fibroblast growth medium 2 (PromoCell C-23020) according to manufacture instruction. Collagen synthesis was inducted using the same cocktail as for the WI-38 cells.

Primary human dermal fibroblasts (HDF) (PromoCell C-12302) were maintained in PromoCell's proprietary Fibroblast Growth Medium 2 (ready-to-use, Cat. C-23020). For collagen synthesis induction, cells were seeded on experimental plates for 24 hours followed by addition of collagen induction cocktail. Tested compounds were added together with induction.

### Protein Synthesis Monitoring (PSM) Assays

Cy3 and Cy5 Labeled tRNA, bulk or specific, are transfected with 0.4 μl HiPerFect (Qiagen) per 384 well. First, HiPerFect is mixed with DMEM and incubated for 5 minutes; next, 8 nanograms Cy3-labeled tRNAPro and 8 ng Cy5-labled tRNAGly (or 8 ng each Cy3 and Cy5-labelled bulk tRNAare diluted in 1×PBS and then added to the HiPerFect:DMEM cocktail and incubated at room temperature for 20 minutes. The transfection mixture is dispersed automatically into 384-well black plates. Cells are then seeded at 3,500 cells per well in DMEM-10% FBS-1% pencillin-Streptomycin-1% L-Glutamine. Plates are incubated at 37° C., 5% CO₂ overnight. Twenty-four hours after transfection collagen production is stimulated with collagen induction cocktail, and then compounds are added at a final concentration of 30 uM. After an additional 24 hours incubation, cells are fixed with 4% paraformaldehyde and images are captured with Operetta microscope (Perkin Elmer) using ×20 high NA objective lens.

### Metabolic Labeling Assay

Synchronized WI-38 cells are seeded at 3,500 cells per well in DMEM-10% FBS-1% pencillin-Streptomycin-1% L-Glutamine. Plates are incubated at 37° C., 5% CO₂ overnight. The collagen production is stimulated with collagen induction cocktail, and then compounds are added at a final concentration of 30 uM. After 20 hours of incubation, the growth medium is aspirated, and cell washed twice with HBSS. Metabolic labeling medium DMEM (-Cys -Met)-10% dialyzed FBS-1% pencillin-Streptomycin-1% L-Glutamine was added to the cells for 30 minutes. Then medium was replaced by metabolic labeling medium containing 25 μM L-Azidohomoalanine (AHA, ThermoFisher) and incubated for 4 hours at 37° C., 5% CO₂. Cells are washed by HBSS at 37° C. for 15 minutes before fixing with 4% paraformaldehyde. Cells are washed twice with 3% BSA in PBS before permeabilization with 0.5% Triton X-100 in PBS for 20 minutes. The AHA staining with Alexa Fluor™ 555 alkyne is performed according to the manufacture instruction. Images are captured with Operetta microscope (Perkin Elmer) using ×20 high NA objective lens.

### Collagen-1 Immunofluorescence Assay

Cells in 96-well or 384-well plates were fixed for 20 min in 4% paraformaldehyde (PFA, ENCO, Cat. sc-281692). Following two washes with 1×PBS, cells were treated with hydrogen peroxide (Acros, Cat: 7722-84-1) for 10 minutes and then washed twice with 1×PBS. Cells were then incubated over-night at 4° C. with Anti-Collagen I (Sigma-Aldrich, Cat: C 2456) antibody and washed three times with 1×PBS. Cells were then incubated with a suitable secondary fluorescently-tagged antibody and nuclei stained with DAPI, for 1 hour, and then washed 3 times with 1×PBS.

Cell images were taken with Operetta (Perkin Elmer, USA), a wide-field fluorescence microscope at 20× magnification. After acquisition, the images were transferred to Columbus software (Perkin-Elmer) for image analysis. In Columbus, cells were identified by their nucleus, using the “Find Nuceli” module and cytoplasm was detected based on the secondary antibody channel. Subsequently, the fluorescent signal was enumerated in the identified cell region. Data was exported to a data analysis and visualization software, Tibco Spotfire, USA.

### Fluorescent In Situ Hybridization (FISH) Assay

WI-38 cells were grown in 384-wells plates (Perkin Elmer, Cat. 6057300) and fixed for 20 min in 4% paraformaldehyde (PFA, ENCO, Cat. sc-281692), and left overnight in 70% ethanol at 4° C. The next day, the cells were washed with 1× PBS and then incubated for 10 min in 10% formamide in 10% saline-sodium citrate (SSC). Huorescently labeled DNA probes that target the COL1 (Cy5, Biosearch Technologies, Cat. SMF-1063-5) and GAPDH (Cy3, Biosearch Technologies, Cat. VSMF-2150-5) mRNAs were hybridized overnight at 37° C. in a dark chamber in 10% formamide. The next day, cells were washed twice with 10% formamide for 30 min. Next, nuclei were counterstained with DAPI (SIGMA, Cat. 5MG-D9542) and then washed twice with 1× PBS. FISH experiments were performed according to the probes manufacturer's protocol for adherent cells.

Following RNA FISH experiments, images of cells were taken with Operetta (Perkin Elmer, USA), a wide-field fluorescence microscope at 20× magnification. After acquisition, the images were transferred to Columbus software for image analysis. In Columbus, cells were identified by their nucleus, using the “Find Nuceli” module, cytoplasm was detected based on the FISH-channel, and single mRNAs in the cytoplasm and transcription sites in the nucleus were detected using “Find Spots” module. Subsequently, fluorescent signals were collected for each channel in the identified regions, nucleus, cytoplasm and spots. Data was exported to a data analysis and visualization software, Tibco Spotfire, USA.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1-83. (canceled)

84. A compound represented by the structure of formula I: 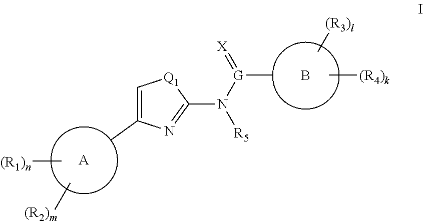
wherein
A ring is a single or fused aromatic or heteroaromatic ring system (e.g., phenyl, thiophene, imidazole, pyrazole, pyrimidine, 2-, 3- or 4-pyridine, benzimidazole, indole, benzothiazole, benzooxazole, imidazopyridin, pyrazolopyridine, pyrrolopyridine, pyridazine, or pyrazine), or a single or fused C₃-C₁₀ cycloalkyl (e.g. pyrrolidin-2-one) or a single or fused C₃-C₁₀ heterocyclic ring (e.g., morpholine, piperidine, piperazine, tetrahydro-2H-pyran, azetidine, pyrrolidin-2-one);
B ring is a single or fused heteroaromatic ring system (e.g., pyrimidine, 2-, 3- or 4-pyridine, pyridazine or pyrazine, thiophene, thiazole, pyrrole, imidazole, indazole), or a single or fused C₃-C₁₀ cycloalkyl (e.g. bicyclo[1.1.1]pentyl, cyclobutyl, cyclohexyl, cyclopentyl) or a single or fused C₃-C₁₀ heterocyclic ring (e.g., morpholine, piperidine, piperazine, tetrahydro-2H-pyran, azetidine, pyrrolidin-2-one);
R₁ is F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl) or substituted or unsubstituted benzyl;
R₂ is H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl) or substituted or unsubstituted benzyl;
or R₂ and R₁ are joined together to form a 5 or 6 membered substituted or unsubstituted, aliphatic or aromatic, carbocyclic (e.g., benzene) or heterocyclic (e.g., 1,4-dioxane, 2,3-dihydro-1,4-dioxine, dioxol, dioxolpyridine) ring;
R₃ is F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)₂₀—CH₃), C(O)N(R₁₀)(R₁₁) (e.g., C(O)-piperidine, C(O)-pyrrolidine, C(O)N(CH₃)₂, C(O)-piperazine), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, N-methylpiperazine-1-carboxamide piperidin-4-ol, piperidin-3-ol, morpholine, 3-methylmorpholine, 3-hydroxypiperidine, tetrahydro-2H-pyrane, tetrahydro-2H-thiopyran 1,1-dioxide, pyrazole, thiazole, imidazole, pyrrolidine, pyrrolidinone, octahydropyrrolo[1,2-a]pyrazine, 6-methyl-2,6-diazaspiro[3.3]heptane, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl), substituted or unsubstituted benzyl;
R₄ is H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)₂₀—CH₃), C(O)N(R₁₀)(R₁₁) (e.g., C(O)-piperidine, C(O)-pyrrolidine, C(O)N(CH₃)₂, C(O)-piperazine), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, N-methylpiperazine-1-carboxamide piperidin-4-ol, piperidin-3-ol, morpholine, 3-methylmorpholine, 3-hydroxypiperidine, tetrahydro-2H-pyrane, tetrahydro-2H-thiopyran 1,1-dioxide, pyrazole, thiazole, imidazole, pyrrolidine, pyrrolidinone, octahydropyrrolo[1,2-a]pyrazine, 6-methyl-2,6-diazaspiro[3.3]heptane, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl), substituted or unsubstituted benzyl;
or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;
R₅ is H, R₂₀, F, Cl, Br, I, CF₃, —C(O)Ph, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), C₁-C₅ linear or branched, substituted or unsubstituted alkyl (e.g., methyl, ethyl), C₁-C₅ linear, branched or cyclic haloalkyl (e.g., CHF₂), substituted or unsubstituted C₃-C₈ cycloalkyl (e.g., cyclopropyl), substituted or unsubstituted C₃-C₈ heterocyclic ring, substituted or unsubstituted aryl, or substituted or unsubstituted benzyl;
Q₁ is NH, S, or 0;
G=X is C═O, C═S, S═O or SO₂;
R is H, OH, F, Cl, Br, I, CN, CF₃, NO₂, NH₂, NH(R₁₀) (e.g., NH(CH₃)), N(R₁₀)(R₁₁), R₂₀, C₁-C₅ linear or branched, C₁-C₅ substituted or unsubstituted alkyl (e.g., methyl, ethyl, CH₂CH₂OH, CH₂CH₂OCH₃), R₈—R₁₀ (e.g., CH₂—OH, CH₂CH₂—OH), C(O)—R₁₀ (e.g., C(O)-methylpyrroldine, C(O)-methylpiperidine, C(O)—CH₃), C₁-C₅ substituted or unsubstituted C(O)-alkyl (e.g., C(O)—CH₂CH₂—OCH₃, C(O)—CH₃, C(O)—CH₂—N(CH₃)₂, C(O)—CH₂—CH₂—N(CH₃)₂, C(O)—CH₂—OH), C(O)—R₈—R₁₀ (e.g., C(O)—CH₂CH₂—OH), C(O)-substituted or unsubstituted C₃-C₈ heterocyclic ring (e.g., C(O)-methylpyrroldine, C(O)-methylpiperidine), C₁-C₅ substituted or unsubstituted SO₂-alkyl (e.g., SO₂—CH₃), C₁-C₅ substituted or unsubstituted C(O)—NH-alkyl (e.g., C(O)—NH—CH₃), C₁-C₅ linear or branched C(O)—O-alkyl (e.g., C(O)—O-tBu), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), or substituted or unsubstituted heteroaryl (e.g., pyridine (2, 3, and 4-pyridine); or
two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;
R₈ is [CH₂]ₚ
wherein p is between 1 and 10 (e.g., 2);
R₉ is [CH]_{q}, [C]_{q}
wherein q is between 2 and 10;
R₁₀ and R₁₁ are each independently H, OH, substituted or unsubstituted C₁-C₅ linear or branched alkyl (e.g., methyl, ethyl, CH₂—CH₂—O—CH₃), C₁-C₅ linear or branched alkoxy (e.g., O—CH₃), substituted or unsubstituted C₃-C₈ heterocyclic ring (e.g., 1-(methylsulfonyl)piperidine, 1-(methylsulfonyl)piperazine, tetrahydro-2H-pyrane, morpholine, thiomorpholine 1,1-dioxide, methyl-pyrrolidine, methyl-piperidine), C(O)-alkyl, or S(O)₂-alkyl;
or R₁₀ and R₁₁ are joined to form a substituted or unsubstituted C₃-C₈ heterocyclic ring (e.g., morpholine, piperazine, piperidine, pyrrolidine, 1-methylpyrrolidin-2-one, oxetane, azetidine, 1-methylazetidine);
R₂₀ is represented by the following structure: 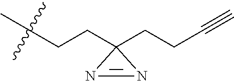 [SMILES: *CCC1(CCC#C)N=N1]
wherein substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof;
n and l are each independently an integer between 1 and 3 (e.g., 1 or 2);
m and k are each independently an integer between 0 and 3 (e.g., 0);
or
a compound represented by any one of the following structures:

| Compound | |
|---|---|
| Number | Compound Structure |
| 305 | 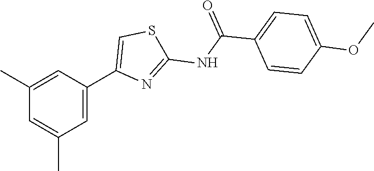 |
| 306 | 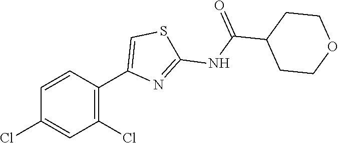 [SMILES: O=C(Nc1nc(-c2ccc(Cl)cc2Cl)cs1)C1CCOCC1] |
| 312 | 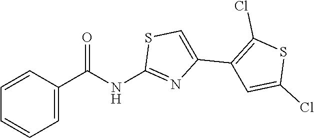 [SMILES: O=C(Nc1nc(-c2cc(Cl)sc2Cl)cs1)c1ccccc1] |
| 314 | 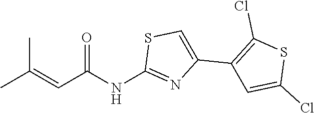 [SMILES: CC(C)=CC(=O)Nc1nc(-c2cc(Cl)sc2Cl)cs1] |
| 315 | 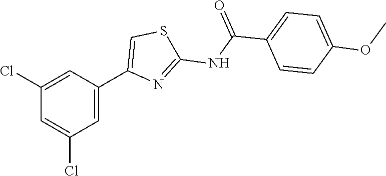 [SMILES: COc1ccc(C(=O)Nc2nc(-c3cc(Cl)cc(Cl)c3)cs2)cc1] |
| 316 | 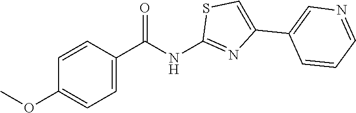 [SMILES: COc1ccc(C(=O)Nc2nc(-c3cccnc3)cs2)cc1] |
| 321 | 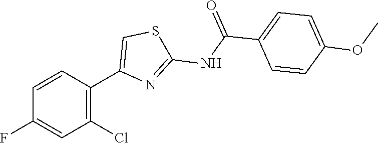 [SMILES: COc1ccc(C(=O)Nc2nc(-c3ccc(F)cc3Cl)cs2)cc1] |
| 322 | 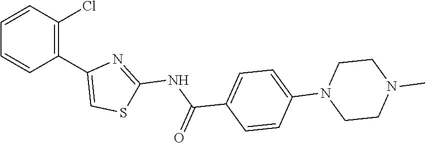 [SMILES: CN1CCN(c2ccc(C(=O)Nc3nc(-c4ccccc4Cl)cs3)cc2)CC1] |
| 323 | 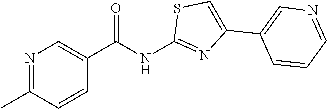 [SMILES: Cc1ccc(C(=O)Nc2nc(-c3cccnc3)cs2)cn1] |
| 326 | 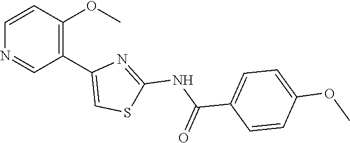 [SMILES: COc1ccc(C(=O)Nc2nc(-c3cnccc3OC)cs2)cc1] |
| 327 | 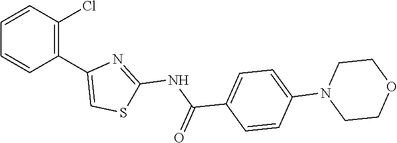 [SMILES: O=C(Nc1nc(-c2ccccc2Cl)cs1)c1ccc(N2CCOCC2)cc1] |
| 328 | 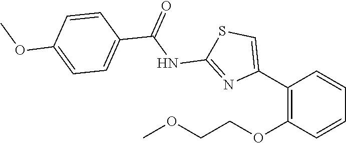 [SMILES: COCCOc1ccccc1-c1csc(NC(=O)c2ccc(OC)cc2)n1] |
| 331 | 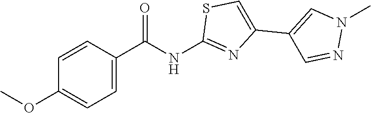 |
| 332 | 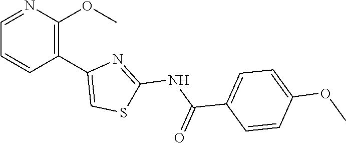 [SMILES: COc1ccc(C(=O)Nc2nc(-c3cccnc3OC)cs2)cc1] |
| 333 | 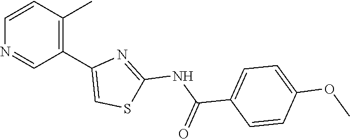 [SMILES: COc1ccc(C(=O)Nc2nc(-c3cnccc3C)cs2)cc1] |
| 334 | 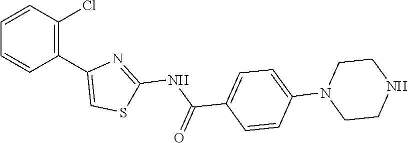 [SMILES: O=C(Nc1nc(-c2ccccc2Cl)cs1)c1ccc(N2CCNCC2)cc1] |
| 335 | 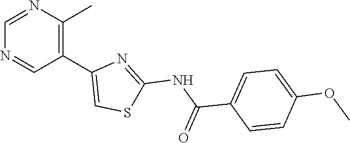 [SMILES: COc1ccc(C(=O)Nc2nc(-c3cncnc3C)cs2)cc1] |
| 336 | 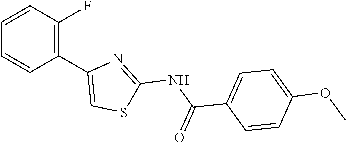 [SMILES: COc1ccc(C(=O)Nc2nc(-c3ccccc3F)cs2)cc1] |
| 337 | 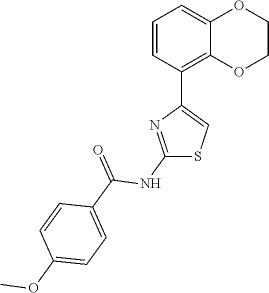 [SMILES: COc1ccc(C(=O)Nc2nc(-c3cccc4c3OCCO4)cs2)cc1] |
| 338 | 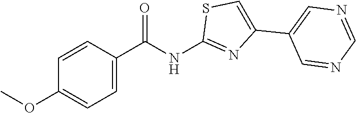 [SMILES: COc1ccc(C(=O)Nc2nc(-c3cncnc3)cs2)cc1] |
| 340 | 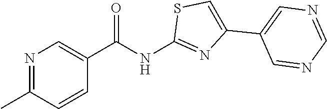 [SMILES: Cc1ccc(C(=O)Nc2nc(-c3cncnc3)cs2)cn1] |
| 343 | 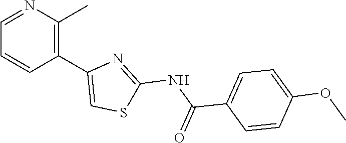 [SMILES: COc1ccc(C(=O)Nc2nc(-c3cccnc3C)cs2)cc1] |
| 351 | 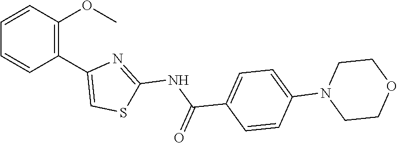 [SMILES: COc1ccccc1-c1csc(NC(=O)c2ccc(N3CCOCC3)cc2)n1] |
| 353 | 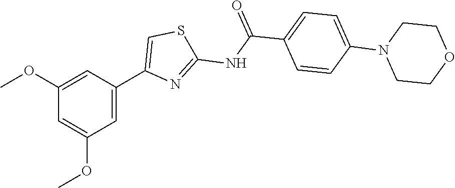 [SMILES: COc1cc(OC)cc(-c2csc(NC(=O)c3ccc(N4CCOCC4)cc3)n2)c1] |
| 354 | 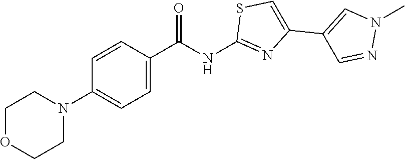 [SMILES: Cn1cc(-c2csc(NC(=O)c3ccc(N4CCOCC4)cc3)n2)cn1] |
| 357 | 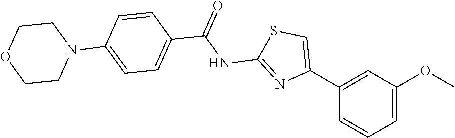 [SMILES: COc1cccc(-c2csc(NC(=O)c3ccc(N4CCOCC4)cc3)n2)c1] |
| 361 | 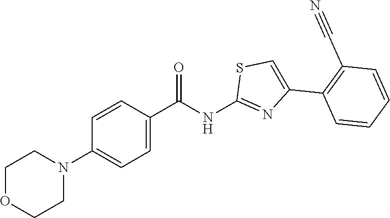 [SMILES: N#Cc1ccccc1-c1csc(NC(=O)c2ccc(N3CCOCC3)cc2)n1] |
| 365 | 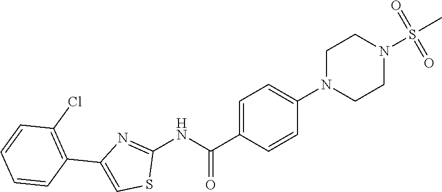 [SMILES: CS(=O)(=O)N1CCN(c2ccc(C(=O)Nc3nc(-c4ccccc4Cl)cs3)cc2)CC1] |
| 368 | 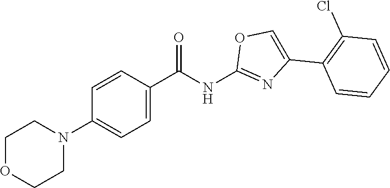 [SMILES: O=C(Nc1nc(-c2ccccc2Cl)co1)c1ccc(N2CCOCC2)cc1] |
| 369 | 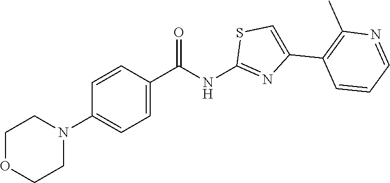 [SMILES: Cc1ncccc1-c1csc(NC(=O)c2ccc(N3CCOCC3)cc2)n1] |
| 376 | 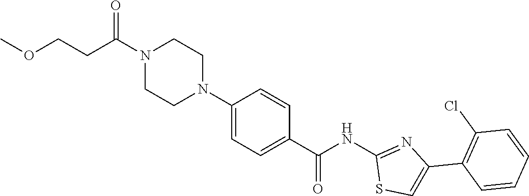 [SMILES: COCCC(=O)N1CCN(c2ccc(C(=O)Nc3nc(-c4ccccc4Cl)cs3)cc2)CC1] |
| 377 | 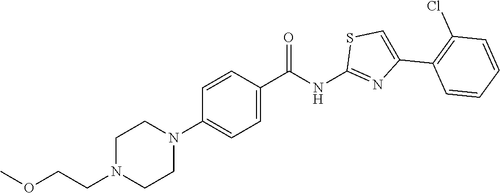 [SMILES: COCCN1CCN(c2ccc(C(=O)Nc3nc(-c4ccccc4Cl)cs3)cc2)CC1] |
| 378 | 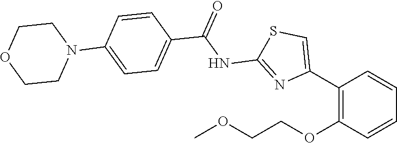 [SMILES: COCCOc1ccccc1-c1csc(NC(=O)c2ccc(N3CCOCC3)cc2)n1] |
| 379 | 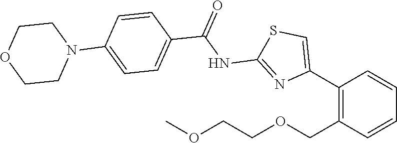 [SMILES: COCCOCc1ccccc1-c1csc(NC(=O)c2ccc(N3CCOCC3)cc2)n1] |
| 380 | 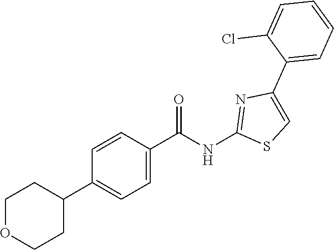 [SMILES: O=C(Nc1nc(-c2ccccc2Cl)cs1)c1ccc(C2CCOCC2)cc1] |
| 382 | 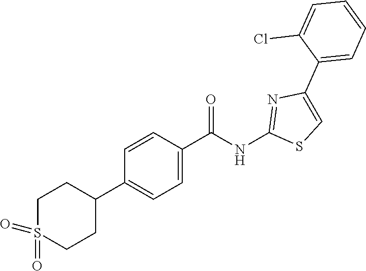 [SMILES: O=C(Nc1nc(-c2ccccc2Cl)cs1)c1ccc(C2CCS(=O)(=O)CC2)cc1] |
| 384 | 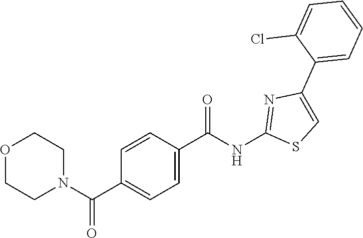 [SMILES: O=C(Nc1nc(-c2ccccc2Cl)cs1)c1ccc(C(=O)N2CCOCC2)cc1] |
| 385 | 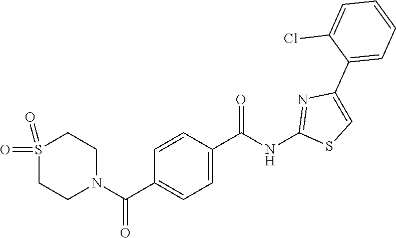 [SMILES: O=C(Nc1nc(-c2ccccc2Cl)cs1)c1ccc(C(=O)N2CCS(=O)(=O)CC2)cc1] |
| 386 | 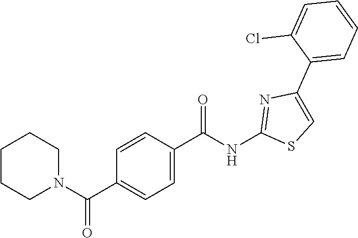 [SMILES: O=C(Nc1nc(-c2ccccc2Cl)cs1)c1ccc(C(=O)N2CCCCC2)cc1] |
| 387 | 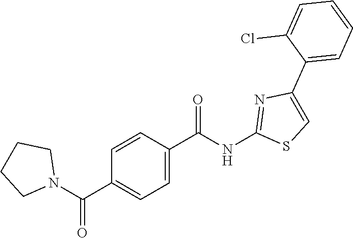 [SMILES: O=C(Nc1nc(-c2ccccc2Cl)cs1)c1ccc(C(=O)N2CCCC2)cc1] |
| 388 | 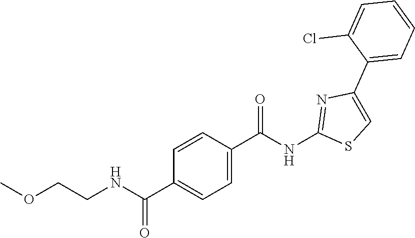 [SMILES: COCCNC(=O)c1ccc(C(=O)Nc2nc(-c3ccccc3Cl)cs2)cc1] |
| 389 | 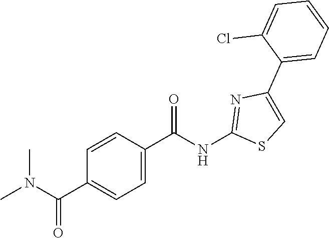 [SMILES: CN(C)C(=O)c1ccc(C(=O)Nc2nc(-c3ccccc3Cl)cs2)cc1] |
| 390 | 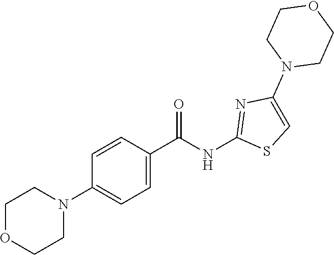 [SMILES: O=C(Nc1nc(N2CCOCC2)cs1)c1ccc(N2CCOCC2)cc1] |
| 391 | 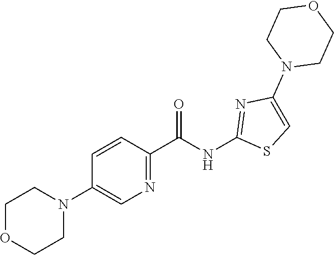 [SMILES: O=C(Nc1nc(N2CCOCC2)cs1)c1ccc(N2CCOCC2)cn1] |
| 392 | 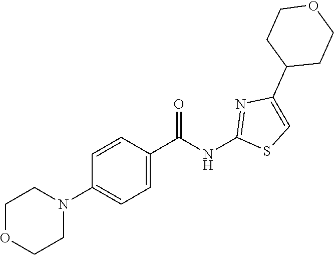 [SMILES: O=C(Nc1nc(C2CCOCC2)cs1)c1ccc(N2CCOCC2)cc1] |
| 393 | 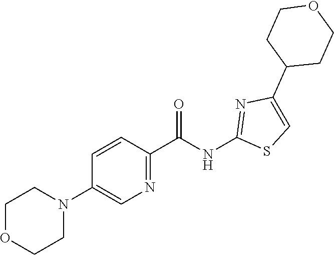 [SMILES: O=C(Nc1nc(C2CCOCC2)cs1)c1ccc(N2CCOCC2)cn1] |
| 394 | 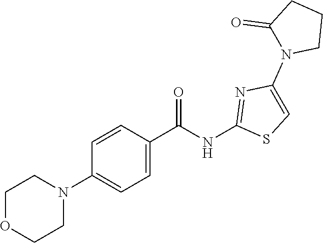 [SMILES: O=C(Nc1nc(N2CCCC2=O)cs1)c1ccc(N2CCOCC2)cc1] |
| 395 | 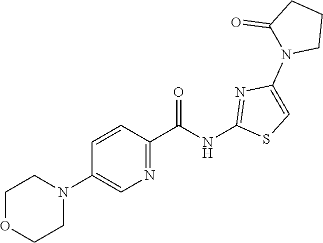 [SMILES: O=C(Nc1nc(N2CCCC2=O)cs1)c1ccc(N2CCOCC2)cn1] |
| 399 | 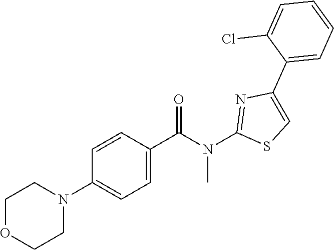 [SMILES: CN(C(=O)c1ccc(N2CCOCC2)cc1)c1nc(-c2ccccc2Cl)cs1] |
| 405 | 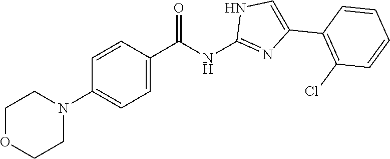 [SMILES: O=C(Nc1nc(-c2ccccc2Cl)c[nH]1)c1ccc(N2CCOCC2)cc1] |
| 408 | 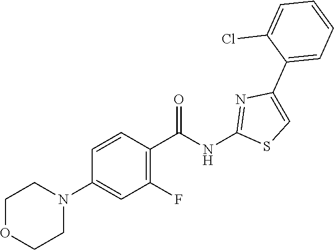 [SMILES: O=C(Nc1nc(-c2ccccc2Cl)cs1)c1ccc(N2CCOCC2)cc1F] |
| 409 | 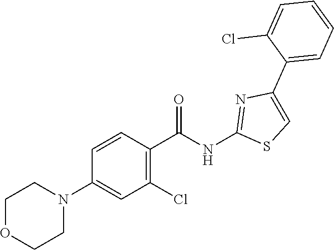 [SMILES: O=C(Nc1nc(-c2ccccc2Cl)cs1)c1ccc(N2CCOCC2)cc1Cl] |
| 410 | 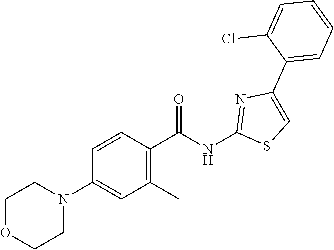 [SMILES: Cc1cc(N2CCOCC2)ccc1C(=O)Nc1nc(-c2ccccc2Cl)cs1] |
| 411 | 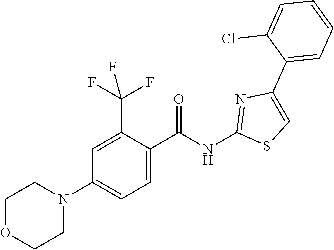 [SMILES: O=C(Nc1nc(-c2ccccc2Cl)cs1)c1ccc(N2CCOCC2)cc1C(F)(F)F] |
| 412 | 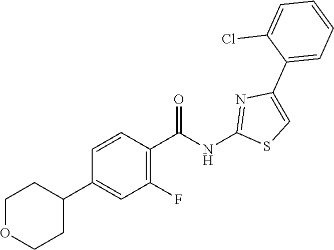 [SMILES: O=C(Nc1nc(-c2ccccc2Cl)cs1)c1ccc(C2CCOCC2)cc1F] |
| 413 | 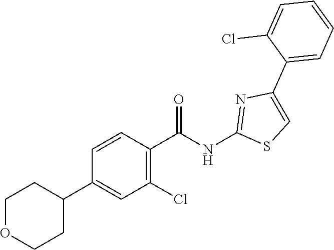 [SMILES: O=C(Nc1nc(-c2ccccc2Cl)cs1)c1ccc(C2CCOCC2)cc1Cl] |
| 414 | 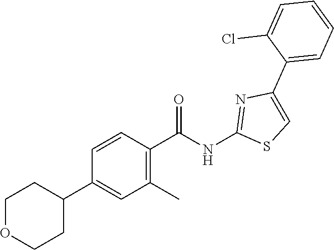 [SMILES: Cc1cc(C2CCOCC2)ccc1C(=O)Nc1nc(-c2ccccc2Cl)cs1] |
| 415 | 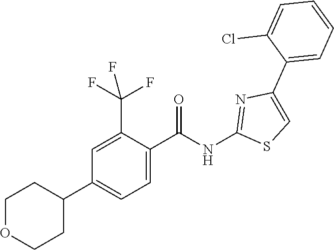 [SMILES: O=C(Nc1nc(-c2ccccc2Cl)cs1)c1ccc(C2CCOCC2)cc1C(F)(F)F] |
| 436 | 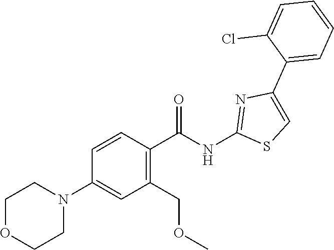 [SMILES: COCc1cc(N2CCOCC2)ccc1C(=O)Nc1nc(-c2ccccc2Cl)cs1] |
| 441 | 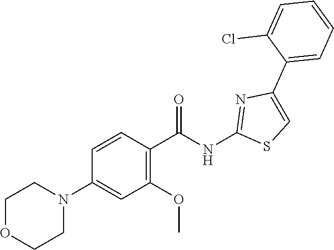 [SMILES: COc1cc(N2CCOCC2)ccc1C(=O)Nc1nc(-c2ccccc2Cl)cs1] |
| 473 | 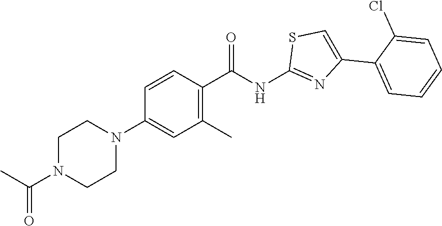 [SMILES: CC(=O)N1CCN(c2ccc(C(=O)Nc3nc(-c4ccccc4Cl)cs3)c(C)c2)CC1] |
| 474 | 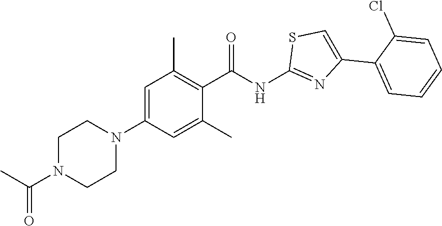 [SMILES: CC(=O)N1CCN(c2cc(C)c(C(=O)Nc3nc(-c4ccccc4Cl)cs3)c(C)c2)CC1] |

or its pharmaceutically acceptable salt, stereoisomer, tautomer, hydrate, N-oxide, prodrug, isotopic variant (e.g., deuterated analog), reverse amide, pharmaceutical product or any combination thereof;
wherein the compound is not N-[4-(2-chlorophenyl)-2-thiazolyl]-6-methyl-3-pyridinecarboxamide, or N-[4-(2-chlorophenyl)-2-thiazolyl]-4-(4-morpholinyl)-2-pyridinecarboxamide.

85. The compound of claim 84, represented by the following structures:

| Compound | |
|---|---|
| Number | Compound Structure |
| 317 | 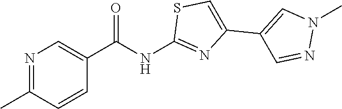 |
| 319 | 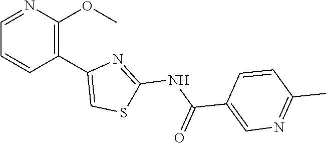 [SMILES: COc1ncccc1-c1csc(NC(=O)c2ccc(C)nc2)n1] |
| 320 | 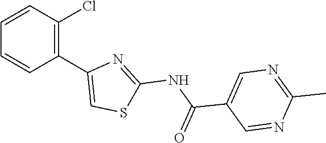 [SMILES: Cc1ncc(C(=O)Nc2nc(-c3ccccc3Cl)cs2)cn1] |
| 324 | 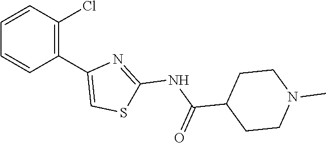 [SMILES: CN1CCC(C(=O)Nc2nc(-c3ccccc3Cl)cs2)CC1] |
| 329 | 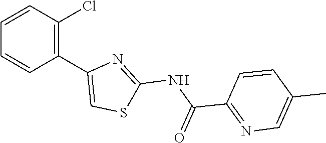 [SMILES: Cc1ccc(C(=O)Nc2nc(-c3ccccc3Cl)cs2)nc1] |
| 330 | 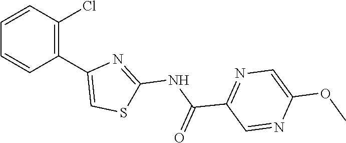 [SMILES: COc1cnc(C(=O)Nc2nc(-c3ccccc3Cl)cs2)cn1] |
| 341 | 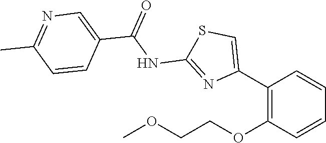 [SMILES: COCCOc1ccccc1-c1csc(NC(=O)c2ccc(C)nc2)n1] |
| 342 | 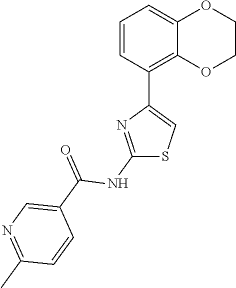 [SMILES: Cc1ccc(C(=O)Nc2nc(-c3cccc4c3OCCO4)cs2)cn1] |
| 344 | 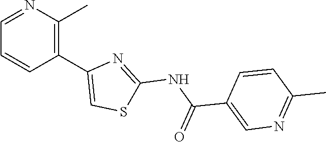 [SMILES: Cc1ccc(C(=O)Nc2nc(-c3cccnc3C)cs2)cn1] |
| 345 | 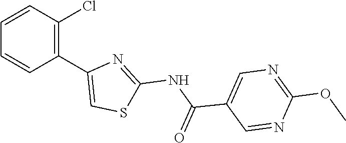 [SMILES: COc1ncc(C(=O)Nc2nc(-c3ccccc3Cl)cs2)cn1] |
| 346 | 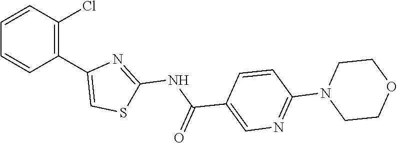 [SMILES: O=C(Nc1nc(-c2ccccc2Cl)cs1)c1ccc(N2CCOCC2)nc1] |
| 347 | 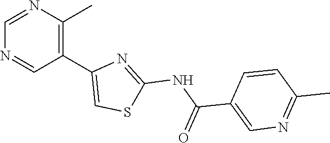 [SMILES: Cc1ccc(C(=O)Nc2nc(-c3cncnc3C)cs2)cn1] |
| 348 | 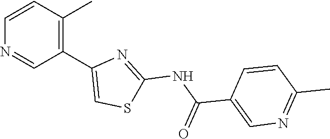 [SMILES: Cc1ccc(C(=O)Nc2nc(-c3cnccc3C)cs2)cn1] |
| 349 | 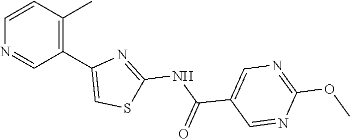 [SMILES: COc1ncc(C(=O)Nc2nc(-c3cnccc3C)cs2)cn1] |
| 350 | 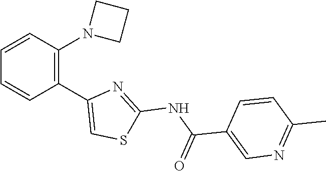 [SMILES: Cc1ccc(C(=O)Nc2nc(-c3ccccc3N3CCC3)cs2)cn1] |
| 355 | 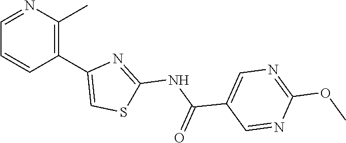 [SMILES: COc1ncc(C(=O)Nc2nc(-c3cccnc3C)cs2)cn1] |
| 356 | 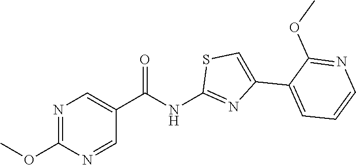 [SMILES: COc1ncc(C(=O)Nc2nc(-c3cccnc3OC)cs2)cn1] |
| 358 | 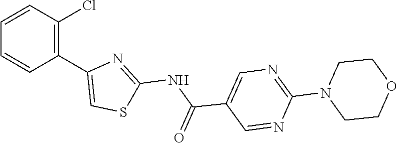 [SMILES: O=C(Nc1nc(-c2ccccc2Cl)cs1)c1cnc(N2CCOCC2)nc1] |
| 360 | 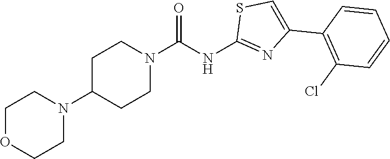 [SMILES: O=C(Nc1nc(-c2ccccc2Cl)cs1)N1CCC(N2CCOCC2)CC1] |
| 363 | 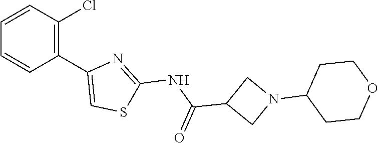 [SMILES: O=C(Nc1nc(-c2ccccc2Cl)cs1)C1CN(C2CCOCC2)C1] |
| 366 | 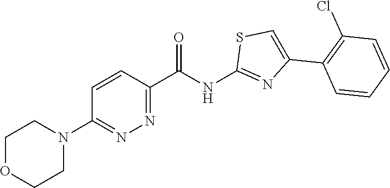 [SMILES: O=C(Nc1nc(-c2ccccc2Cl)cs1)c1ccc(N2CCOCC2)nn1] |
| 367 | 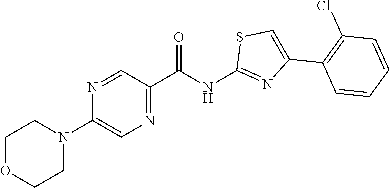 [SMILES: O=C(Nc1nc(-c2ccccc2Cl)cs1)c1cnc(N2CCOCC2)cn1] |
| 371 | 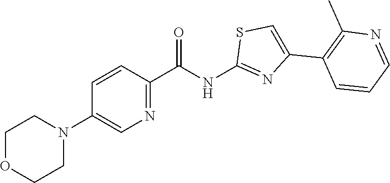 [SMILES: Cc1ncccc1-c1csc(NC(=O)c2ccc(N3CCOCC3)cn2)n1] |
| 372 | 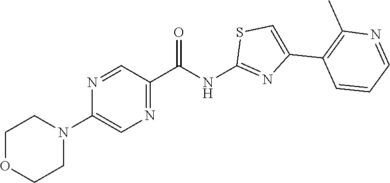 [SMILES: Cc1ncccc1-c1csc(NC(=O)c2cnc(N3CCOCC3)cn2)n1] |
| 373 | 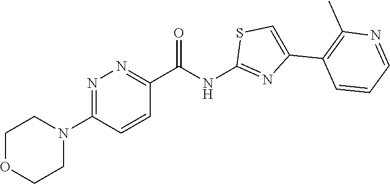 [SMILES: Cc1ncccc1-c1csc(NC(=O)c2ccc(N3CCOCC3)nn2)n1] |
| 374 | 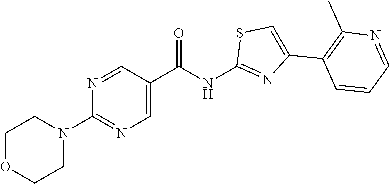 [SMILES: Cc1ncccc1-c1csc(NC(=O)c2cnc(N3CCOCC3)nc2)n1] |
| 375 | 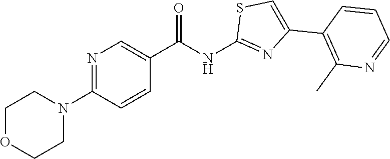 [SMILES: Cc1ncccc1-c1csc(NC(=O)c2ccc(N3CCOCC3)nc2)n1] |
| 381 | 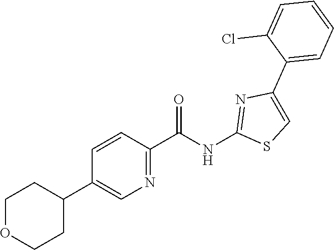 [SMILES: O=C(Nc1nc(-c2ccccc2Cl)cs1)c1ccc(C2CCOCC2)cn1] |
| 383 | 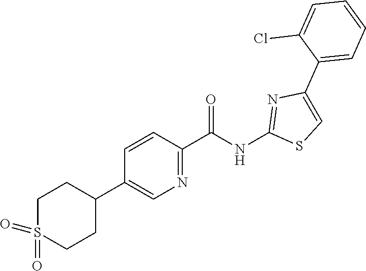 [SMILES: O=C(Nc1nc(-c2ccccc2Cl)cs1)c1ccc(C2CCS(=O)(=O)CC2)cn1] |
| 391 | 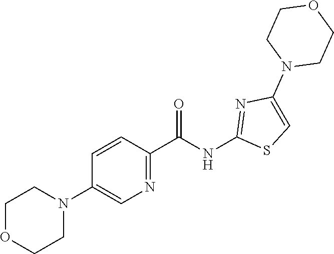 [SMILES: O=C(Nc1nc(N2CCOCC2)cs1)c1ccc(N2CCOCC2)cn1] |
| 393 | 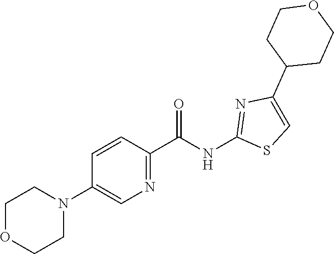 [SMILES: O=C(Nc1nc(C2CCOCC2)cs1)c1ccc(N2CCOCC2)cn1] |
| 395 | 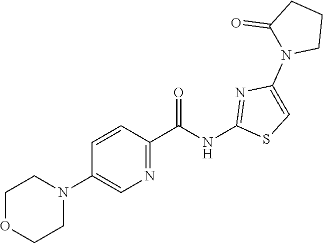 [SMILES: O=C(Nc1nc(N2CCCC2=O)cs1)c1ccc(N2CCOCC2)cn1] |
| 400 | 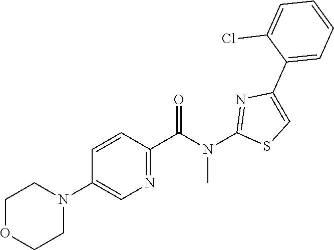 [SMILES: CN(C(=O)c1ccc(N2CCOCC2)cn1)c1nc(-c2ccccc2Cl)cs1] |
| 403 | 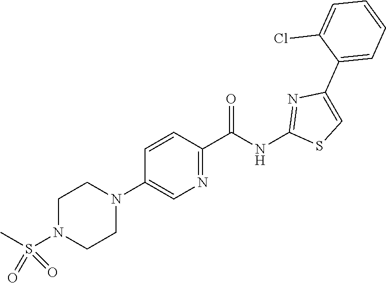 [SMILES: CS(=O)(=O)N1CCN(c2ccc(C(=O)Nc3nc(-c4ccccc4Cl)cs3)nc2)CC1] |
| 404 | 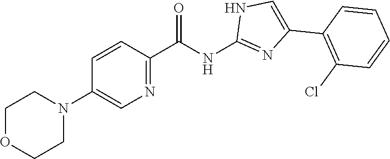 [SMILES: O=C(Nc1nc(-c2ccccc2Cl)c[nH]1)c1ccc(N2CCOCC2)cn1] |
| 416 | 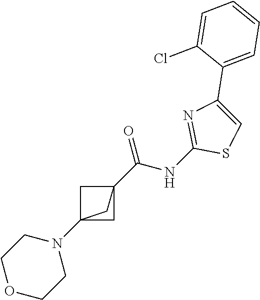 [SMILES: O=C(Nc1nc(-c2ccccc2Cl)cs1)C12CC(N3CCOCC3)(C1)C2] |
| 417 | 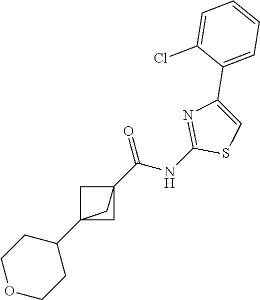 [SMILES: O=C(Nc1nc(-c2ccccc2Cl)cs1)C12CC(C3CCOCC3)(C1)C2] |
| 418 | 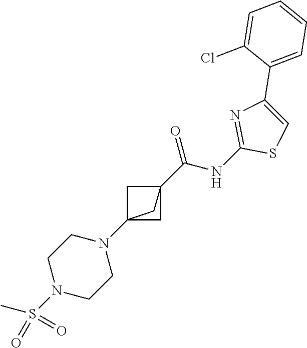 [SMILES: CS(=O)(=O)N1CCN(C23CC(C(=O)Nc4nc(-c5ccccc5Cl)cs4)(C2)C3)CC1] |
| 419 | 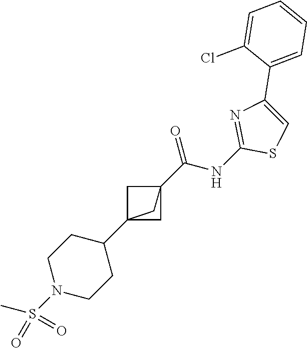 [SMILES: CS(=O)(=O)N1CCC(C23CC(C(=O)Nc4nc(-c5ccccc5Cl)cs4)(C2)C3)CC1] |
| 420 | 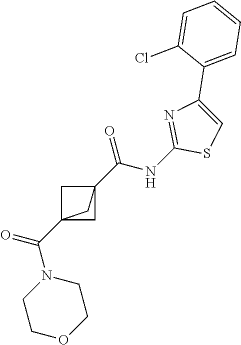 [SMILES: O=C(Nc1nc(-c2ccccc2Cl)cs1)C12CC(C(=O)N3CCOCC3)(C1)C2] |
| 421 | 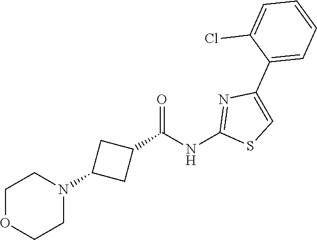 |
| 422 | 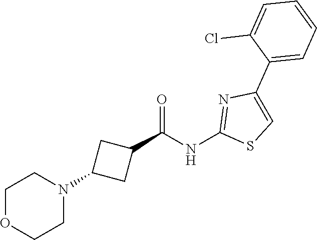 |
| 423 | 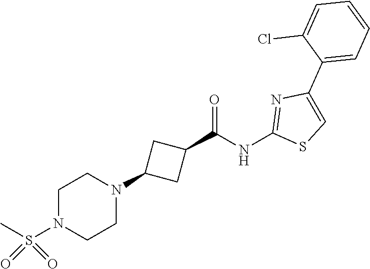 [SMILES: CS(=O)(=O)N1CCN([C@H]2C[C@@H](C(=O)Nc3nc(-c4ccccc4Cl)cs3)C2)CC1] |
| 424 | 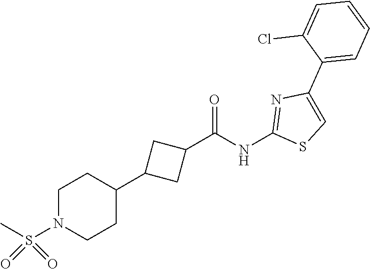 [SMILES: CS(=O)(=O)N1CCC(C2CC(C(=O)Nc3nc(-c4ccccc4Cl)cs3)C2)CC1] |
| 425 | 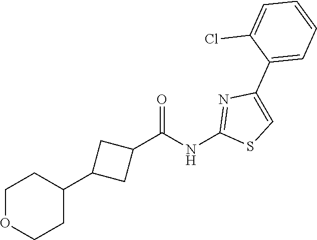 [SMILES: O=C(Nc1nc(-c2ccccc2Cl)cs1)C1CC(C2CCOCC2)C1] |
| 426 | 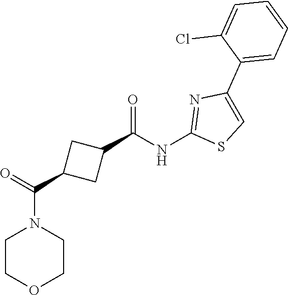 |
| 427 | 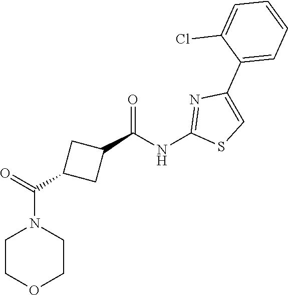 |
| 428 | 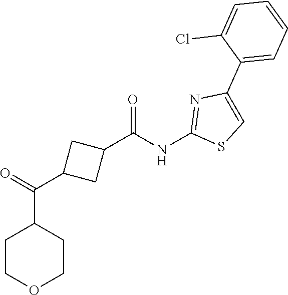 [SMILES: O=C(Nc1nc(-c2ccccc2Cl)cs1)C1CC(C(=O)C2CCOCC2)C1] |
| 429 | 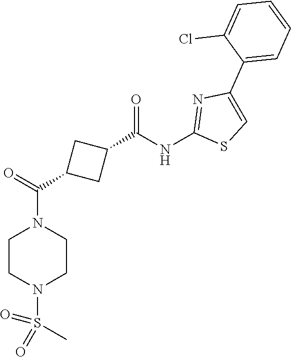 [SMILES: CS(=O)(=O)N1CCN(C(=O)[C@H]2C[C@@H](C(=O)Nc3nc(-c4ccccc4Cl)cs3)C2)CC1] |
| 430 | 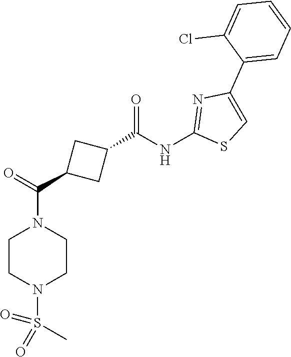 [SMILES: CS(=O)(=O)N1CCN(C(=O)[C@H]2C[C@H](C(=O)Nc3nc(-c4ccccc4Cl)cs3)C2)CC1] |
| 431 | 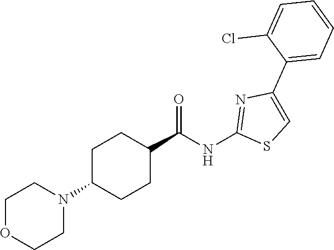 |
| 432 | 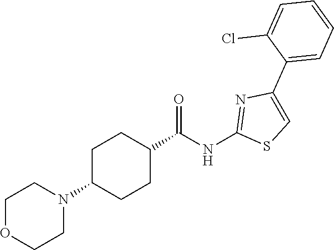 |
| 433 | 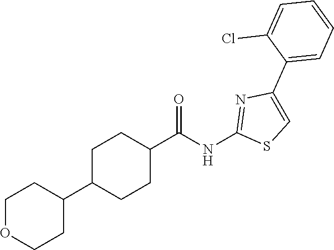 [SMILES: O=C(Nc1nc(-c2ccccc2Cl)cs1)C1CCC(C2CCOCC2)CC1] |
| 434 | 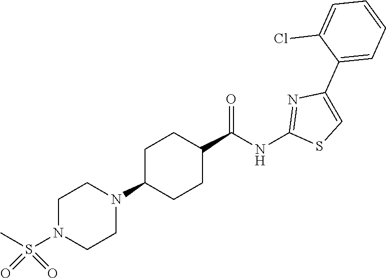 |
| 435 | 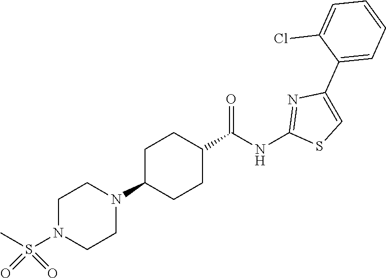 |
| 437 | 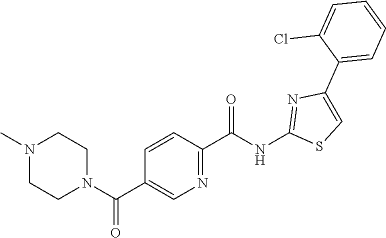 [SMILES: CN1CCN(C(=O)c2ccc(C(=O)Nc3nc(-c4ccccc4Cl)cs3)nc2)CC1] |
| 438 | 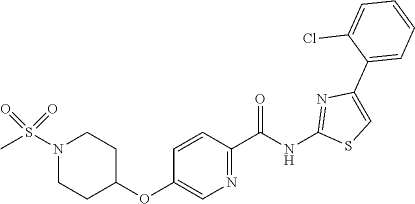 [SMILES: CS(=O)(=O)N1CCC(Oc2ccc(C(=O)Nc3nc(-c4ccccc4Cl)cs3)nc2)CC1] |
| 439 | 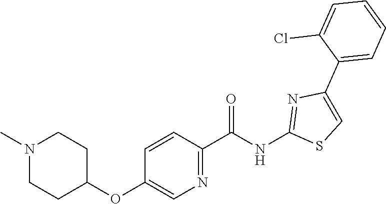 [SMILES: CN1CCC(Oc2ccc(C(=O)Nc3nc(-c4ccccc4Cl)cs3)nc2)CC1] |
| 440 | 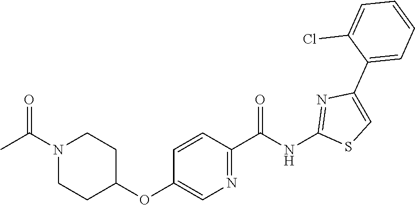 [SMILES: CC(=O)N1CCC(Oc2ccc(C(=O)Nc3nc(-c4ccccc4Cl)cs3)nc2)CC1] |
| 442 | 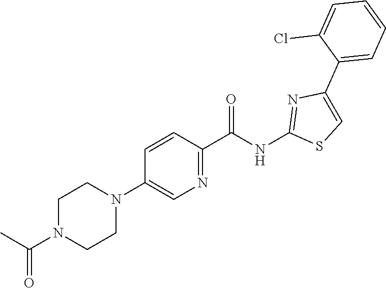 [SMILES: CC(=O)N1CCN(c2ccc(C(=O)Nc3nc(-c4ccccc4Cl)cs3)nc2)CC1] |
| 443 | 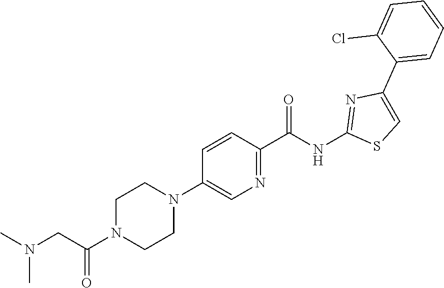 [SMILES: CN(C)CC(=O)N1CCN(c2ccc(C(=O)Nc3nc(-c4ccccc4Cl)cs3)nc2)CC1] |
| 444 | 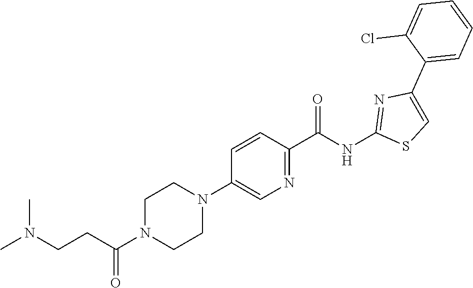 [SMILES: CN(C)CCC(=O)N1CCN(c2ccc(C(=O)Nc3nc(-c4ccccc4Cl)cs3)nc2)CC1] |
| 445 | 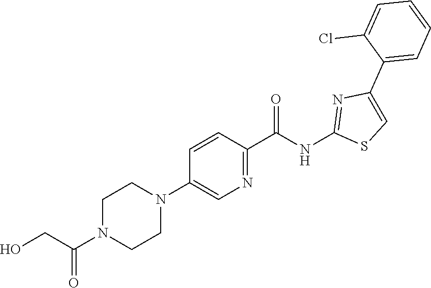 [SMILES: O=C(Nc1nc(-c2ccccc2Cl)cs1)c1ccc(N2CCN(C(=O)CO)CC2)cn1] |
| 446 | 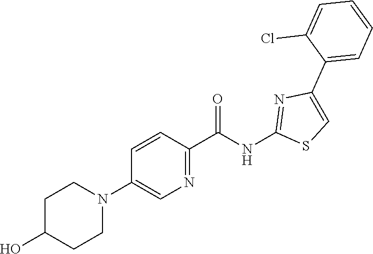 [SMILES: O=C(Nc1nc(-c2ccccc2Cl)cs1)c1ccc(N2CCC(O)CC2)cn1] |
| 447 | 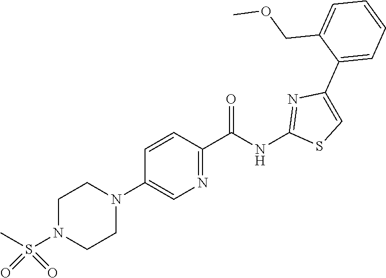 [SMILES: COCc1ccccc1-c1csc(NC(=O)c2ccc(N3CCN(S(C)(=O)=O)CC3)cn2)n1] |
| 448 | 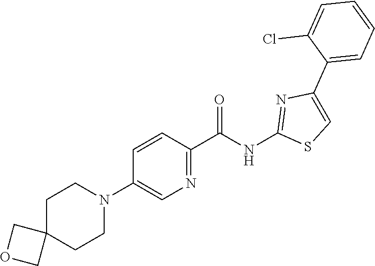 [SMILES: O=C(Nc1nc(-c2ccccc2Cl)cs1)c1ccc(N2CCC3(CC2)COC3)cn1] |
| 449 | 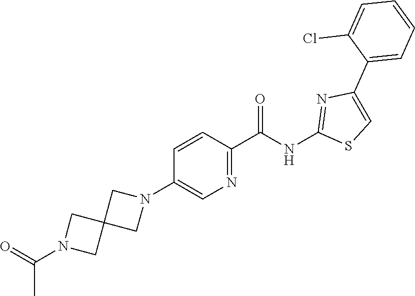 [SMILES: CC(=O)N1CC2(C1)CN(c1ccc(C(=O)Nc3nc(-c4ccccc4Cl)cs3)nc1)C2] |
| 450 | 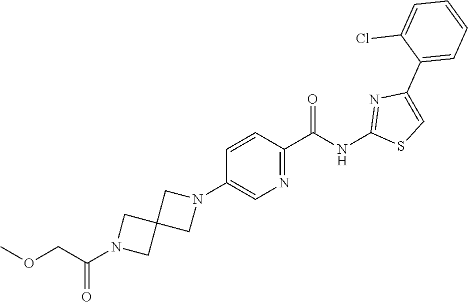 [SMILES: COCC(=O)N1CC2(C1)CN(c1ccc(C(=O)Nc3nc(-c4ccccc4Cl)cs3)nc1)C2] |
| 451 | 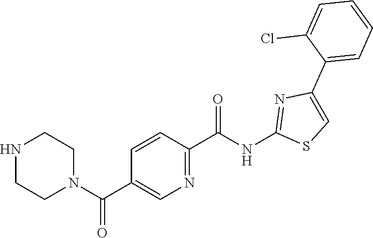 [SMILES: O=C(Nc1nc(-c2ccccc2Cl)cs1)c1ccc(C(=O)N2CCNCC2)cn1] |
| 452 | 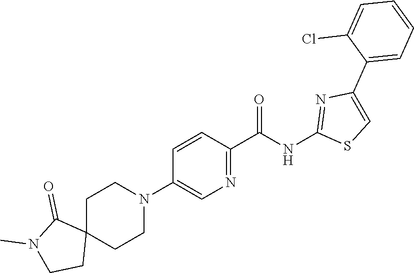 [SMILES: CN1CCC2(CCN(c3ccc(C(=O)Nc4nc(-c5ccccc5Cl)cs4)nc3)CC2)C1=O] |
| 453 | 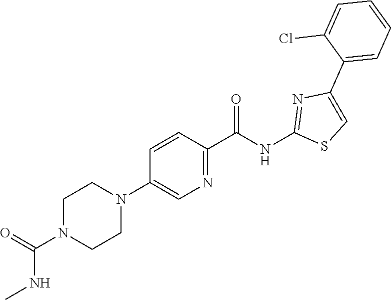 [SMILES: CNC(=O)N1CCN(c2ccc(C(=O)Nc3nc(-c4ccccc4Cl)cs3)nc2)CC1] |
| 454 | 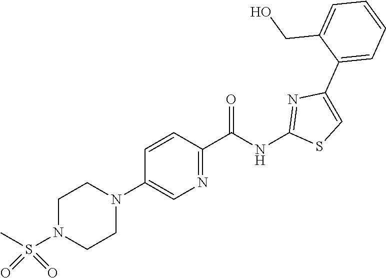 [SMILES: CS(=O)(=O)N1CCN(c2ccc(C(=O)Nc3nc(-c4ccccc4CO)cs3)nc2)CC1] |
| 455 | 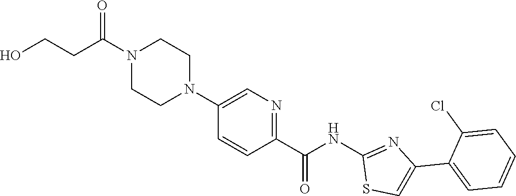 [SMILES: O=C(Nc1nc(-c2ccccc2Cl)cs1)c1ccc(N2CCN(C(=O)CCO)CC2)cn1] |
| 456 | 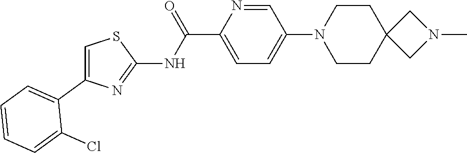 [SMILES: CN1CC2(CCN(c3ccc(C(=O)Nc4nc(-c5ccccc5Cl)cs4)nc3)CC2)C1] |
| 457 | 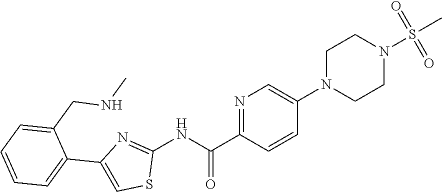 [SMILES: CNCc1ccccc1-c1csc(NC(=O)c2ccc(N3CCN(S(C)(=O)=O)CC3)cn2)n1] |
| 458 | 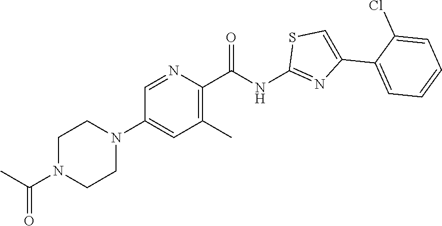 [SMILES: CC(=O)N1CCN(c2cnc(C(=O)Nc3nc(-c4ccccc4Cl)cs3)c(C)c2)CC1] |
| 459 | 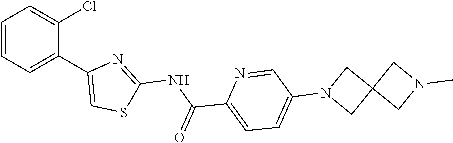 [SMILES: CN1CC2(C1)CN(c1ccc(C(=O)Nc3nc(-c4ccccc4Cl)cs3)nc1)C2] |
| 460 | 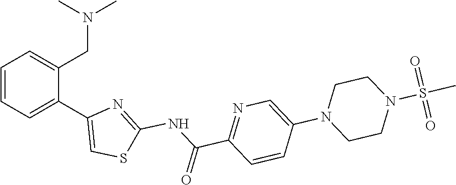 [SMILES: CN(C)Cc1ccccc1-c1csc(NC(=O)c2ccc(N3CCN(S(C)(=O)=O)CC3)cn2)n1] |
| 461 | 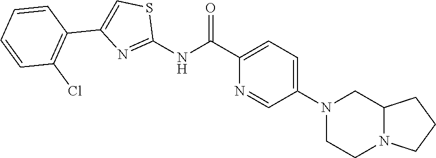 [SMILES: O=C(Nc1nc(-c2ccccc2Cl)cs1)c1ccc(N2CCN3CCCC3C2)cn1] |
| 462 | 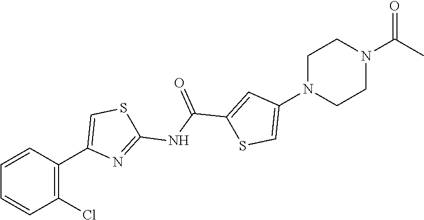 [SMILES: CC(=O)N1CCN(c2csc(C(=O)Nc3nc(-c4ccccc4Cl)cs3)c2)CC1] |
| 463 | 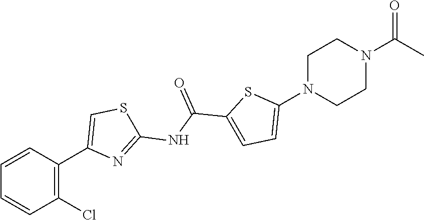 [SMILES: CC(=O)N1CCN(c2ccc(C(=O)Nc3nc(-c4ccccc4Cl)cs3)s2)CC1] |
| 464 | 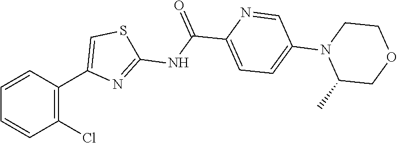 [SMILES: C[C@H]1COCCN1c1ccc(C(=O)Nc2nc(-c3ccccc3Cl)cs2)nc1] |
| 465 | 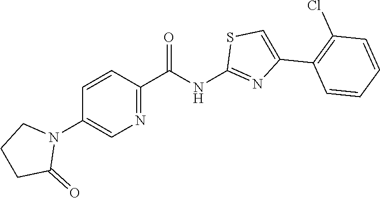 [SMILES: O=C(Nc1nc(-c2ccccc2Cl)cs1)c1ccc(N2CCCC2=O)cn1] |
| 466 | 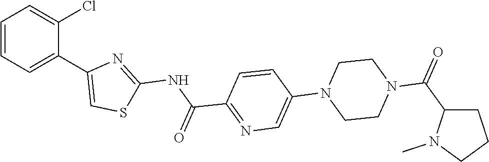 [SMILES: CN1CCCC1C(=O)N1CCN(c2ccc(C(=O)Nc3nc(-c4ccccc4Cl)cs3)nc2)CC1] |
| 467 | 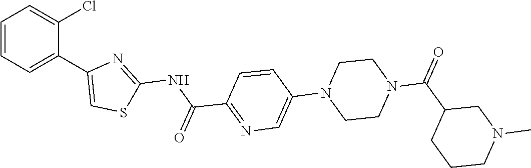 [SMILES: CN1CCCC(C(=O)N2CCN(c3ccc(C(=O)Nc4nc(-c5ccccc5Cl)cs4)nc3)CC2)C1] |
| 468 | 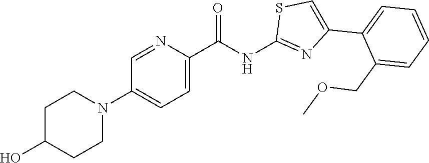 [SMILES: COCc1ccccc1-c1csc(NC(=O)c2ccc(N3CCC(O)CC3)cn2)n1] |
| 469 | 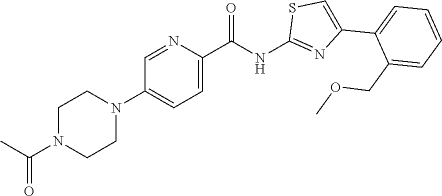 [SMILES: COCc1ccccc1-c1csc(NC(=O)c2ccc(N3CCN(C(C)=O)CC3)cn2)n1] |
| 470 | 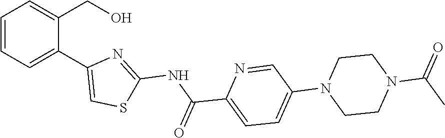 [SMILES: CC(=O)N1CCN(c2ccc(C(=O)Nc3nc(-c4ccccc4CO)cs3)nc2)CC1] |
| 471 | 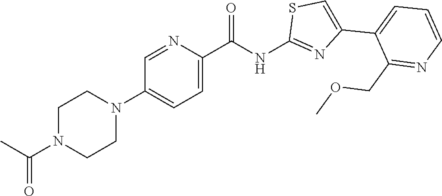 [SMILES: COCc1ncccc1-c1csc(NC(=O)c2ccc(N3CCN(C(C)=O)CC3)cn2)n1] |
| 472 | 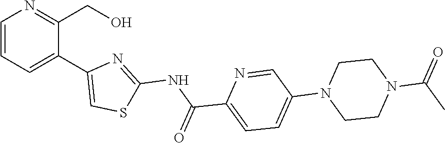 [SMILES: CC(=O)N1CCN(c2ccc(C(=O)Nc3nc(-c4cccnc4CO)cs3)nc2)CC1] |
| 475 | 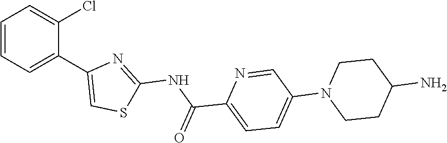 [SMILES: NC1CCN(c2ccc(C(=O)Nc3nc(-c4ccccc4Cl)cs3)nc2)CC1] |
| 476 | 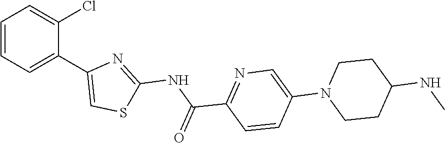 [SMILES: CNC1CCN(c2ccc(C(=O)Nc3nc(-c4ccccc4Cl)cs3)nc2)CC1] |
| 477 | 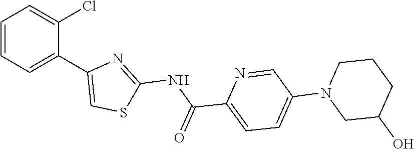 [SMILES: O=C(Nc1nc(-c2ccccc2Cl)cs1)c1ccc(N2CCCC(O)C2)cn1] |
| 478 | 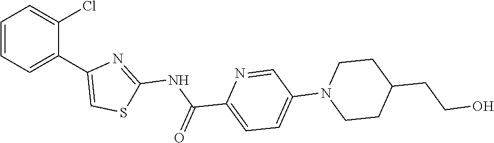 [SMILES: O=C(Nc1nc(-c2ccccc2Cl)cs1)c1ccc(N2CCC(CCO)CC2)cn1] |
| 479 | 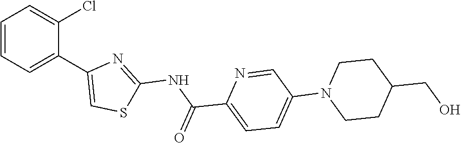 [SMILES: O=C(Nc1nc(-c2ccccc2Cl)cs1)c1ccc(N2CCC(CO)CC2)cn1] |
| 480 | 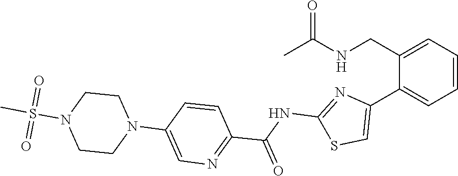 [SMILES: CC(=O)NCc1ccccc1-c1csc(NC(=O)c2ccc(N3CCN(S(C)(=O)=O)CC3)cn2)n1] |
| 481 | 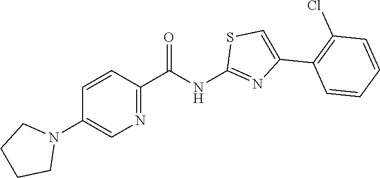 [SMILES: O=C(Nc1nc(-c2ccccc2Cl)cs1)c1ccc(N2CCCC2)cn1] |
| 482 | 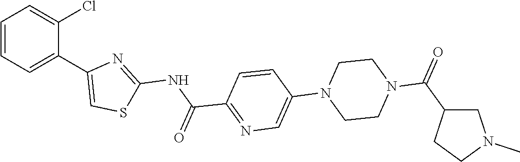 [SMILES: CN1CCC(C(=O)N2CCN(c3ccc(C(=O)Nc4nc(-c5ccccc5Cl)cs4)nc3)CC2)C1] |
| 483 | 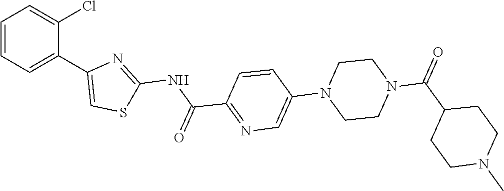 [SMILES: CN1CCC(C(=O)N2CCN(c3ccc(C(=O)Nc4nc(-c5ccccc5Cl)cs4)nc3)CC2)CC1] |
| 484 | 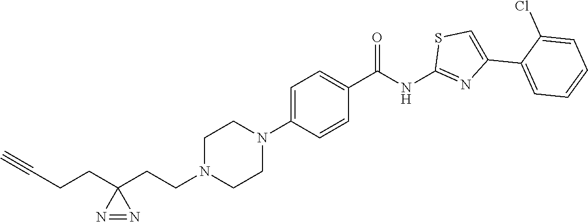 [SMILES: C#CCCC1(CCN2CCN(c3ccc(C(=O)Nc4nc(-c5ccccc5Cl)cs4)cc3)CC2)N=N1] |
| 485 | 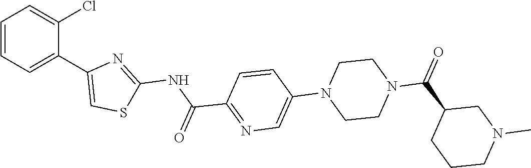 [SMILES: CN1CCC[C@@H](C(=O)N2CCN(c3ccc(C(=O)Nc4nc(-c5ccccc5Cl)cs4)nc3)CC2)C1] |
| 486 | 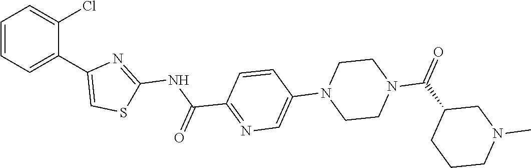 [SMILES: CN1CCC[C@H](C(=O)N2CCN(c3ccc(C(=O)Nc4nc(-c5ccccc5Cl)cs4)nc3)CC2)C1] |
| 487 | 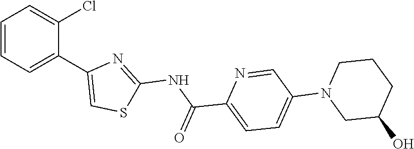 [SMILES: O=C(Nc1nc(-c2ccccc2Cl)cs1)c1ccc(N2CCC[C@@H](O)C2)cn1] |
| 488 | 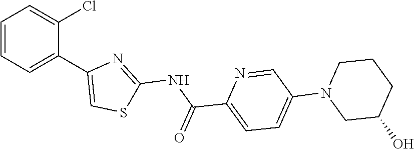 [SMILES: O=C(Nc1nc(-c2ccccc2Cl)cs1)c1ccc(N2CCC[C@H](O)C2)cn1] |

86. The compound of claim 84, represented by the structure of formula II: 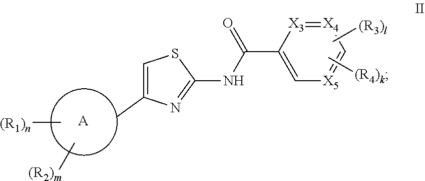
by the structure of formula III: 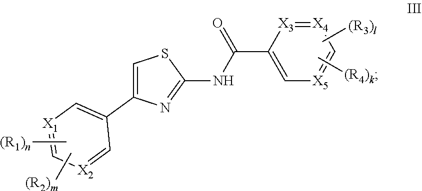
by the structure of formula IV: 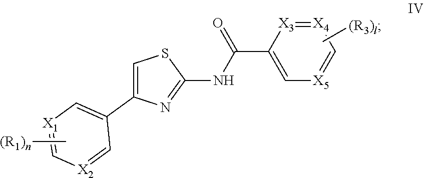
or by the structure of formula V: 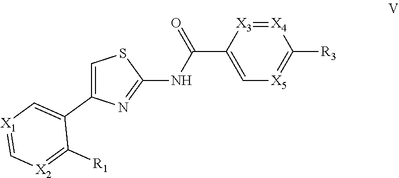
wherein
X₁, X₂ X₃, X₄ and X₅ are each independently C or N; and
wherein at least one of X₃, X₄ and X₅ is N.

87. The compound of claim 86, wherein at least two of X₃, X₄ and X₅ are N.

88. The compound of claim 86,
wherein R₁ is not H;
wherein R₁ is in the ortho position,
wherein R₁ is Cl, —R₈—O—R₁₀, or CH₂—O—CH₃;
wherein R₃ is in the para position,
wherein R₃ is N(R₁₀)(R₁₁) (e.g., morpholine, piperazine), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, N-methylpiperazine-1-carboxamide piperidin-4-ol, piperidin-3-ol, morpholine, 3-methylmorpholine, 3-hydroxypiperidine, tetrahydro-2H-pyrane, tetrahydro-2H-thiopyran 1,1-dioxide, pyrazole, thiazole, imidazole, pyrrolidine, pyrrolidinone, octahydropyrrolo[1,2-a]pyrazine, 6-methyl-2,6-diazaspiro[3.3]heptane, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), or any combination thereof;
wherein R is H or OH;
wherein R₁₀ is substituted or unsubstituted C₃-C₈ heterocyclic ring, methyl-piperidine, or wherein R₁₀ and R₁₁ are joined to form a substituted or unsubstituted C₃-C₈ heterocyclic ring or 1-methylazetidine;
or any combination thereof.

89. A compound, represented by the structure of formula VI: 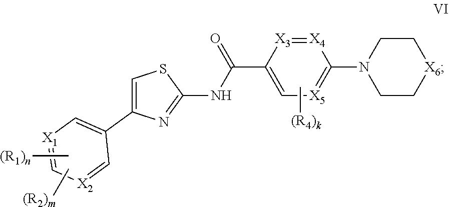
or by the structure of formula VIII: 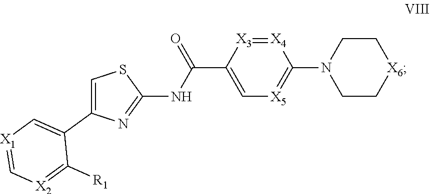
wherein
R₁ and R₂ are each independently H, F, Cl, Br, I, OH, SH, R₈—OH (e.g. CH₂OH), R₈—SH, —R₈—O—R₁₀ (e.g., CH₂—CH₂—O—CH₃, CH₂—O—CH₂—CH₂—O—CH₃, CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—NH—C(O)CH₃, CH₂—N(CH₃)₂), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., azetidine, pyridine), substituted or unsubstituted aryl (e.g., phenyl), or substituted or unsubstituted benzyl;
or R₂ and R₁ are joined together to form a 5 or 6 membered substituted or unsubstituted, aliphatic or aromatic, carbocyclic (e.g., benzene) or heterocyclic (e.g., 1,4-dioxane, 2,3-dihydro-1,4-dioxine, dioxol, dioxolpyridine) ring;
R₄ is H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, N(R₁₀)(R₁₁) (e.g., morpholine, piperazine), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)₂₀—CH₃), C(O)N(R₁₀)(R₁₁) (e.g., C(O)-piperidine, C(O)-pyrrolidine, C(O)N(CH₃)₂, C(O)-piperazine), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy, 1-(methylsulfonyl)piperidin-4-oxy, 1-(methyl)piperidin-4-oxy, 1-(ethanone)piperidin-4-oxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted, single, spirocyclic, fused, or bridged C₃-C₁₀ heterocyclic ring (e.g., piperazine, 1-(2-methoxyethyl)piperazine, 1-, or 4-methylpiperazine, 1- or 4-(methylsulfonyl)piperazine, 1- or 4-(methylsulfonyl)piperidine, 2-methoxy-1-(piperazin-1-yl)ethenone, 1-(piperazin-1-yl)ethanone, 2-(dimethylamino)-1-(piperazin-1-yl)ethanone, 2-(dimethylamino)-1-(piperazin-1-yl)propanone, 2-hydroxy-1-(piperazin-1-yl)ethenone, N-methylpiperazine-1-carboxamide piperidin-4-ol, morpholine, 3-methylmorpholine, 3-hydroxypiperidine, tetrahydro-2H-pyrane, tetrahydro-2H-thiopyran 1,1-dioxide, pyrazole, thiazole, imidazole, pyrrolidine, pyrrolidinone, octahydropyrrolo[1,2-a]pyrazine, 6-methyl-2,6-diazaspiro[3.3]heptane, 2-oxa-7-azaspiro[3.5]nonane, 1-(2,6-diazaspiro[3.3]heptan-2-yl)ethenone, 2-methoxy-1-(2,6-diazaspiro[3.3]heptan-2-yl)ethenone, 2,8-diazaspiro[4.5]decan-1-one, 2-oxa-7-azaspiro[3.5]nonane), substituted or unsubstituted aryl (e.g., phenyl), or substituted or unsubstituted benzyl;
X₁, X₂ X₃, X₄ and X₅ are each independently C or N;
wherein at least one of X₃, X₄ and X₅ is N;
X₆ is O, CH₂, CHR (e.g., CH(OH), CH(NH₂), CH(NH(CH₃))), C(R₁₀)(R₁₁) (e.g., C(H)CH₂CH₂—OH, C(H)CH₂—OH, 1-methylazetidine), NH, N—R (e.g., N—CH₃, N—SO₂—CH₃, N—R₂₀, N—CH₂CH₂—OCH₃) or N—C(O)—R₁₀ (e.g., N—C(O)O-tBu, N—C(O)—CH₂CH₂—OCH₃, N—C(O)—CH₃, N—C(O)—CH₂—N(CH₃)₂, N—C(O)—CH₂—CH₂—N(CH₃)₂, N—C(O)—CH₂—OH, N—C(O)—CH₂CH₂—OH, N—C(O)—NH—CH₃, N—C(O)-1-methyl-2-pyrrolidine, N—C(O)-1-methyl-3-pyrrolidine, N—C(O)-1-methyl-3-piperidine, N—C(O)-1-methyl-4-piperidine);
R is H, OH, F, Cl, Br, I, CN, CF₃, NO₂, NH₂, NH(R₁₀) (e.g., NH(CH₃)), N(R₁₀)(R₁₁), R₂₀, C₁-C₅ linear or branched, C₁-C₅ substituted or unsubstituted alkyl (e.g., methyl, ethyl, CH₂CH₂OH, CH₂CH₂OCH₃), R₈—R₁₀ (e.g., CH₂—OH, CH₂CH₂—OH), C(O)—R₁₀ (e.g., C(O)-methylpyrroldine, C(O)-methylpiperidine, C(O)—CH₃), C₁-C₅ substituted or unsubstituted C(O)-alkyl (e.g., C(O)—CH₂CH₂—OCH₃, C(O)—CH₃, C(O)—CH₂—N(CH₃)₂, C(O)—CH₂—CH₂—N(CH₃)₂, C(O)—CH₂—OH), C(O)—R₈—R₁₀ (e.g., C(O)—CH₂CH₂—OH), C(O)-substituted or unsubstituted C₃-C₈ heterocyclic ring (e.g., C(O)-methylpyrroldine, C(O)-methylpiperidine), C₁-C₅ substituted or unsubstituted SO₂-alkyl (e.g., SO₂—CH₃), C₁-C₅ substituted or unsubstituted C(O)—NH-alkyl (e.g., C(O)—NH—CH₃), C₁-C₅ linear or branched C(O)—O-alkyl (e.g., C(O)—O-tBu), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or
two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;
R₈ is [CH₂]ₚ
wherein p is between 1 and 10 (e.g., 2);
R₉ is [CH]_{q}, [C]_{q}
wherein q is between 2 and 10;
R₁₀ and R₁₁ are each independently H, OH, substituted or unsubstituted C₁-C₅ linear or branched alkyl (e.g., methyl, ethyl, CH₂—CH₂—O—CH₃), C₁-C₅ linear or branched alkoxy (e.g., O—CH₃), substituted or unsubstituted C₃-C₈ heterocyclic ring (e.g., 1-(methylsulfonyl)piperidine, 1-(methylsulfonyl)piperazine, tetrahydro-2H-pyrane, morpholine, thiomorpholine 1,1-dioxide, methyl-pyrrolidine, methyl-piperidine), C(O)-alkyl, or S(O)₂-alkyl;
or R₁₀ and R₁₁ are joined to form a substituted or unsubstituted C₃-C₈ heterocyclic ring (e.g., morpholine, piperazine, piperidine, pyrrolidine, 1-methylpyrrolidin-2-one, oxetane, azetidine, 1-methylazetidine),
R₂₀ is represented by the following structure: 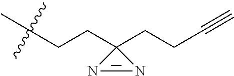 [SMILES: *CCC1(CCC#C)N=N1]
wherein substitutions include: F, Cl, Br, I, OH, SH, CF₃, CN, NO₂, substituted or unsubstituted C₁-C₅ linear or branched alkyl (e.g., methyl, methoxyethyl), substituted or unsubstituted C₁-C₅ linear or branched C(O)-alkyl (e.g., C(O)—CH₃, C(O)—CH₂—O—CH₃), SO₂-alkyl (e.g., SO₂—CH₃), C(O)—NH-alkyl, C₁-C₅ linear or branched alkyl-OH (e.g., C(CH₃)₂CH₂—OH, CH₂CH₂—OH), C₃-C₈ heterocyclic ring (e.g., piperidine), substituted or unsubstituted C₁-C₅ linear or branched alkoxy, N(R)₂, N(R₁₀)(R₁₁), aryl, phenyl, heteroaryl, C₃-C₈ cycloalkyl, halophenyl, (benzyloxy)phenyl or any combination thereof;
n is an integer between 1 and 3 (e.g., 1 or 2);
m and k are each independently an integer between 0 and 2 (e.g., 0);
wherein the compound is not N-[4-(2-chlorophenyl)-2-thiazolyl]-4-(4-morpholinyl)-2-pyridinecarboxamide;
or its pharmaceutically acceptable salt, stereoisomer, tautomer, hydrate, N-oxide, prodrug, isotopic variant (e.g., deuterated analog), reverse amide, pharmaceutical product or any combination thereof.

90. The compound of claim 89, wherein X₆ is CHR, CH(OH), C(R₁₀)(R₁₁), 1-methylazetidine, N—R, N—SO₂—CH₃, N—C(O)—R₁₀, N—C(O)—CH₃, N—C(O)—NH—CH₃, or N—C(O)-1-methyl-3-piperidine;

91. The compound of claim 89, wherein R₁ is not H;
wherein R₁ is in the ortho position,
wherein R₁ is Cl, —R₈—O—R₁₀, or CH₂—O—CH₃;
wherein R is H or OH;
wherein R₁₀ is substituted or unsubstituted C₃-C₈ heterocyclic ring, methyl-piperidine, or wherein R₁₀ and R₁₁ are joined to form a substituted or unsubstituted C₃-C₈ heterocyclic ring or 1-methylazetidine;
or any combination thereof.

92. The compound according to claim 84, wherein the compound is a collagen translation inhibitor.

93. A pharmaceutical composition comprising a compound according to claim 84 and a pharmaceutically acceptable carrier.

94. A method of treating, suppressing, reducing the severity, reducing the risk of developing or inhibiting fibrosis in a subject, comprising administering an effective amount of a compound according to claim 84 to a subject suffering from fibrosis, thereby treating, suppressing, reducing the severity, reducing the risk of developing or inhibiting fibrosis in said subject.

95. The method of claim 94, wherein said fibrosis is a systemic fibrotic disease;
wherein said fibrosis is an organ-specific fibrotic disease;
wherein said fibrosis is primary or secondary fibrosis;
wherein said fibrosis is a result of systemic sclerosis, graft-versus host disease (GVHD), pulmonary fibrosis, autoimmune disorder, tissue injury, inflammation, oxidative stress or any combination thereof;
wherein the fibrosis is hepatic fibrosis, lung fibrosis or dermal fibrosis;
wherein said subject has a liver cirrhosis;
or any combination thereof.

96. The method of claim 95,
wherein said systemic fibrotic disease is systemic sclerosis, multifocal fibrosclerosis (IgG4-associated fibrosis), nephrogenic systemic fibrosis, sclerodermatous graft vs. host disease, or any combination thereof;
wherein said organ-specific fibrotic disease is lung fibrosis, cardiac fibrosis, kidney fibrosis, pulmonary fibrosis, liver and portal vein fibrosis, radiation-induced fibrosis, bladder fibrosis, intestinal fibrosis, peritoneal sclerosis, diffuse fasciitis, wound healing, scaring, or any combination thereof;
wherein the dermal fibrosis is scleroderma;
wherein the dermal fibrosis is a result of a localized or generalized morphea, keloids, hypertrophic scars, familial cutaneous collagenoma, connective tissue nevi of the collagen type, or any combination thereof;
wherein the hepatic fibrosis is a result of hepatic scarring or chronic liver injury;
or any combination thereof.

97. The method of claim 96,
wherein said lung fibrosis is idiopathic pulmonary fibrosis (IPF);
wherein said cardiac fibrosis is hypertension-associated cardiac fibrosis, Post-myocardial infarction, Chagas disease-induced myocardial fibrosis or any combination thereof;
wherein said kidney fibrosis is diabetic and hypertensive nephropathy, urinary tract obstruction-induced kidney fibrosis, inflammatory/autoimmune-induced kidney fibrosis, aristolochic acid nephropathy, polycystic kidney disease, or any combination thereof;
wherein said pulmonary fibrosis is idiopathic pulmonary fibrosis, silica-induced pneumoconiosis (silicosis), asbestos-induced pulmonary fibrosis (asbestosis), chemotherapeutic agent-induced pulmonary fibrosis, or any combination thereof;
wherein said liver and portal vein fibrosis is alcoholic and nonalcoholic liver fibrosis, hepatitis C-induced liver fibrosis, primary biliary cirrhosis, parasite-induced liver fibrosis (schistosomiasis), or any combination thereof;
wherein said diffuse fasciitis is localized scleroderma, keloids, dupuytren's disease, peyronie's disease, myelofibrosis, oral submucous fibrosis, or any combination thereof;
wherein the chronic liver injury results from alcoholism, malnutrition, hemochromatosis, exposure to poisons, toxins or drugs;
or any combination thereof.

98. A method of treating, suppressing, reducing the severity, reducing the risk of developing or inhibiting a disease or condition selected from: lung fibrosis, idiopathic pulmonary fibrosis (IPF), hepato-fibrotic disorder, cirrhosis, alcoholic steatohepatitis (ASH), non-alcoholic steatohepatitis (NASH), alcoholic fatty liver disease (AFLD), non alcoholic fatty liver disease (NAFLD), and an autoimmune disease, in a subject, comprising administering an effective amount of a compound according to claim 84, to a subject suffering from said disease or condition, thereby treating, suppressing, reducing the severity, reducing the risk of developing or inhibiting said disease or condition.

99. The method of claim 98,
wherein the lung fibrosis is idiopathic pulmonary fibrosis (IPF),
wherein the hepato-fibrotic disorder is a portal hypertension, cirrhosis, congenital hepatic fibrosis or any combination thereof;
wherein the cirrhosis is a result of hepatitis or alcoholism; or combination thereof;
or any combination thereof.

100. A method of treating, suppressing, reducing the severity, reducing the risk of developing or inhibiting a disease or condition selected from: lung fibrosis, idiopathic pulmonary fibrosis (IPF), hepato-fibrotic disorder, cirrhosis, alcoholic steatohepatitis (ASH), non-alcoholic steatohepatitis (NASH), alcoholic fatty liver disease (AFLD), non alcoholic fatty liver disease (NAFLD), and an autoimmune disease, in a subject, comprising administering an effective amount of a compound represented by any one of the following structures:

| Compound | |
|---|---|
| Number | Compound Structure |
| 300 | 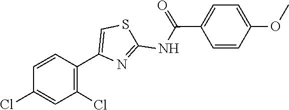 [SMILES: COc1ccc(C(=O)Nc2nc(-c3ccc(Cl)cc3Cl)cs2)cc1] |
| 301 | 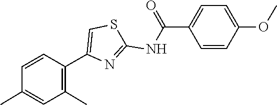 |
| 302 | 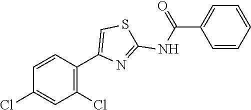 [SMILES: O=C(Nc1nc(-c2ccc(Cl)cc2Cl)cs1)c1ccccc1] |
| 303 | 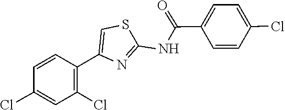 [SMILES: O=C(Nc1nc(-c2ccc(Cl)cc2Cl)cs1)c1ccc(Cl)cc1] |
| 304 | 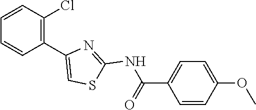 [SMILES: COc1ccc(C(=O)Nc2nc(-c3ccccc3Cl)cs2)cc1] |
| 306 | 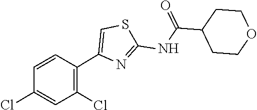 [SMILES: O=C(Nc1nc(-c2ccc(Cl)cc2Cl)cs1)C1CCOCC1] |
| 307 | 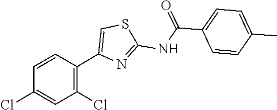 [SMILES: Cc1ccc(C(=O)Nc2nc(-c3ccc(Cl)cc3Cl)cs2)cc1] |
| 308 | 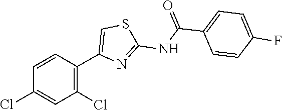 [SMILES: O=C(Nc1nc(-c2ccc(Cl)cc2Cl)cs1)c1ccc(F)cc1] |
| 310 | 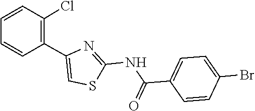 [SMILES: O=C(Nc1nc(-c2ccccc2Cl)cs1)c1ccc(Br)cc1] |
| 311 | 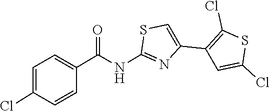 [SMILES: O=C(Nc1nc(-c2cc(Cl)sc2Cl)cs1)c1ccc(Cl)cc1] |
| 312 | 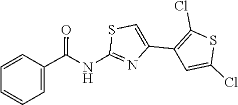 [SMILES: O=C(Nc1nc(-c2cc(Cl)sc2Cl)cs1)c1ccccc1] |
| 313 | 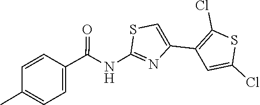 [SMILES: Cc1ccc(C(=O)Nc2nc(-c3cc(Cl)sc3Cl)cs2)cc1] |
| 314 | 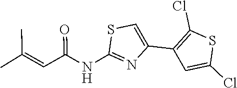 [SMILES: CC(C)=CC(=O)Nc1nc(-c2cc(Cl)sc2Cl)cs1] |
| 318 | 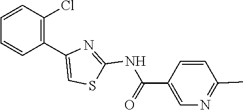 [SMILES: Cc1ccc(C(=O)Nc2nc(-c3ccccc3Cl)cs2)cn1] |
| 325 | 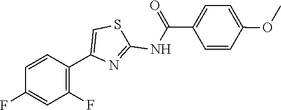 [SMILES: COc1ccc(C(=O)Nc2nc(-c3ccc(F)cc3F)cs2)cc1] |
| 339 | 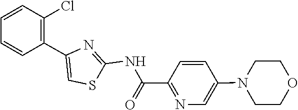 [SMILES: O=C(Nc1nc(-c2ccccc2Cl)cs1)c1ccc(N2CCOCC2)cn1] |
| 352 | 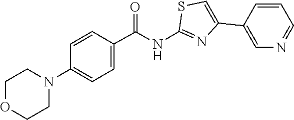 [SMILES: O=C(Nc1nc(-c2cccnc2)cs1)c1ccc(N2CCOCC2)cc1] |

to a subject suffering from said disease or condition, thereby treating, suppressing, reducing the severity, reducing the risk of developing or inhibiting said disease or condition in said subject.

101. The method of claim 100,
wherein said fibrosis is a systemic fibrotic disease;
wherein said fibrosis is an organ-specific fibrotic disease;
wherein said fibrosis is primary or secondary fibrosis;
wherein said fibrosis is a result of systemic sclerosis, graft-versus host disease (GVHD), pulmonary fibrosis, autoimmune disorder, tissue injury, inflammation, oxidative stress or any combination thereof;
wherein said subject has a liver cirrhosis;
wherein the fibrosis is hepatic fibrosis, lung fibrosis or dermal fibrosis;
wherein the lung fibrosis is idiopathic pulmonary fibrosis (IPF);
wherein the hepato-fibrotic disorder is a portal hypertension, cirrhosis, congenital hepatic fibrosis or any combination thereof;
wherein the cirrhosis is a result of hepatitis or alcoholism;
or any combination thereof.

102. The method of claim 101,
wherein said systemic fibrotic disease is systemic sclerosis, multifocal fibrosclerosis (IgG4-associated fibrosis), nephrogenic systemic fibrosis, sclerodermatous graft vs. host disease, or any combination thereof;
wherein said organ-specific fibrotic disease is lung fibrosis, cardiac fibrosis, kidney fibrosis, pulmonary fibrosis, liver and portal vein fibrosis, radiation-induced fibrosis, bladder fibrosis, intestinal fibrosis, peritoneal sclerosis, diffuse fasciitis, wound healing, scaring, or any combination thereof;
wherein the dermal fibrosis is scleroderma;
wherein the dermal fibrosis is a result of a localized or generalized morphea, keloids, hypertrophic scars, familial cutaneous collagenoma, connective tissue nevi of the collagen type, or any combination thereof;
wherein the hepatic fibrosis is a result of hepatic scarring or chronic liver injury;
or any combination thereof.

103. The method of claim 102,
wherein said lung fibrosis is idiopathic pulmonary fibrosis (IPF);
wherein said cardiac fibrosis is hypertension-associated cardiac fibrosis, Post-myocardial infarction, Chagas disease-induced myocardial fibrosis or any combination thereof;
wherein said kidney fibrosis is diabetic and hypertensive nephropathy, urinary tract obstruction-induced kidney fibrosis, inflammatory/autoimmune-induced kidney fibrosis, aristolochic acid nephropathy, polycystic kidney disease, or any combination thereof;
wherein said pulmonary fibrosis is idiopathic pulmonary fibrosis, silica-induced pneumoconiosis (silicosis), asbestos-induced pulmonary fibrosis (asbestosis), chemotherapeutic agent-induced pulmonary fibrosis, or any combination thereof;
wherein said liver and portal vein fibrosis is alcoholic and nonalcoholic liver fibrosis, hepatitis C-induced liver fibrosis, primary biliary cirrhosis, parasite-induced liver fibrosis (schistosomiasis), or any combination thereof;
wherein said diffuse fasciitis is localized scleroderma, keloids, dupuytren's disease, peyronie's disease, myelofibrosis, oral submucous fibrosis, or any combination thereof;
wherein the chronic liver injury results from alcoholism, malnutrition, hemochromatosis, exposure to poisons, toxins or drugs;
or any combination thereof.
